SEQUENCE LISTING (410 sequences)

SEQ ID NO: 1 - protein, 122 aa, synthetic construct
  REGION 1..122: note = Description of Artificial Sequence: Synthetic polypeptide
QMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARAPQWELVHEAFDIWGQGTMVTVSS

SEQ ID NO: 2 - protein, 108 aa, synthetic construct
  REGION 1..108: note = Description of Artificial Sequence: Synthetic polypeptide
SYVLTQPPSVSVAPGQTARITCGGNNLGSKSVHWYQQKPGQAPVLVVYDDSDRPSWIPERFSGSNSGNTATLTISRGEAGDEADYYCQVWDSSSDHVVFGGGTKLTVL

SEQ ID NO: 3 - protein, 108 aa, synthetic construct
  REGION 1..108: note = Description of Artificial Sequence: Synthetic polypeptide
SYVLTQPPSVSVAPGQTARITCGGNLIGSKLVHWYQQKPGQAPVLVVYDDSDRPSLIPERFSGSNSGNTATLTISRVEAGDEADYYCQVWDSSSDGVVFGGGTKLTVL

SEQ ID NO: 4 - protein, 122 aa, synthetic construct
  REGION 1..122: note = Description of Artificial Sequence: Synthetic polypeptide
QMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARAPQWELTAEAFDIWGQGTMVTVSS

SEQ ID NO: 5 - protein, 108 aa, synthetic construct; identical to SEQ ID NO: 3
  REGION 1..108: note = Description of Artificial Sequence: Synthetic polypeptide

SEQ ID NO: 6 - protein, 122 aa, synthetic construct
  REGION 1..122: note = Description of Artificial Sequence: Synthetic polypeptide
QMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARAPQWELVAEAFDLWGQGTMVTVSS

SEQ ID NO: 7 - protein, 108 aa, synthetic construct
  REGION 1..108: note = Description of Artificial Sequence: Synthetic polypeptide
SYVLTQPPSVSVAPGQTARITCGGNLIGSKLVHWYQQKPGQAPVLVVYDDSDRPSLIPERFSGSNSGNTATLTISRVEAGDEADYYCQVWDTSSDGVVFGGGTKLTVL

SEQ ID NO: 8 - protein, 122 aa, synthetic construct
  REGION 1..122: note = Description of Artificial Sequence: Synthetic polypeptide
QMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARAPQWQLVAEAFDIWGQGTMVTVSS

SEQ ID NO: 9 - protein, 108 aa, synthetic construct
  REGION 1..108: note = Description of Artificial Sequence: Synthetic polypeptide
SYVLTQPPSVSVAPGQTARITCGGNLIGSKLVHWYQQKPGQAPVLVVYDDSDRPSAIPERFSGSNSGNTATLTISRVEAGDEADYYCQVWDTSSDGVVFGGGTKLTVL

SEQ ID NO: 10 - protein, 122 aa, synthetic construct; identical to SEQ ID NO: 8
  REGION 1..122: note = Description of Artificial Sequence: Synthetic polypeptide

SEQ ID NO: 11 - protein, 108 aa, synthetic construct; identical to SEQ ID NO: 7
  REGION 1..108: note = Description of Artificial Sequence: Synthetic polypeptide

SEQ ID NO: 12 - protein, 122 aa, synthetic construct; identical to SEQ ID NO: 6
  REGION 1..122: note = Description of Artificial Sequence: Synthetic polypeptide

SEQ ID NO: 13 - protein, 108 aa, synthetic construct
  REGION 1..108: note = Description of Artificial Sequence: Synthetic polypeptide
SYVLTQPPSVSVAPGQTARITCGGNLLGSKLVHWYQQKPGQAPVLVVYDDSDRPSAIPERFSGSNSGNTATLTISRVEAGDEADYYCQVWDSSSDHVVFGGGTKLTVL

SEQ ID NO: 14 - protein, 122 aa, synthetic construct
  REGION 1..122: note = Description of Artificial Sequence: Synthetic polypeptide
QMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARAPQWELVAEAFDIWGQGTMVTVSS

SEQ ID NO: 15 - protein, 108 aa, synthetic construct
  REGION 1..108: note = Description of Artificial Sequence: Synthetic polypeptide
SYVLTQPPSVSVAPGQTARITCGGNLIGSKLVHWYQQKPGQAPVLVVYDDSDRPSRIPERFSGSNSGNTATLTISRVEAGDEADYYCQVWDSSSDHVVFGGGTKLTVL

SEQ ID NO: 16 - protein, 122 aa, synthetic construct
  REGION 1..122: note = Description of Artificial Sequence: Synthetic polypeptide
QMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARAPQWMLVAEAFDLWGQGTMVTVSS

SEQ ID NO: 17 - protein, 108 aa, synthetic construct; identical to SEQ ID NO: 15
  REGION 1..108: note = Description of Artificial Sequence: Synthetic polypeptide

SEQ ID NO: 18 - protein, 122 aa, synthetic construct
  REGION 1..122: note = Description of Artificial Sequence: Synthetic polypeptide
QMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARAPQWEWVAEAFDLWGQGTMVTVSS

SEQ ID NO: 19 - protein, 108 aa, synthetic construct; identical to SEQ ID NO: 15
  REGION 1..108: note = Description of Artificial Sequence: Synthetic polypeptide

SEQ ID NO: 20 - protein, 122 aa, synthetic construct
  REGION 1..122: note = Description of Artificial Sequence: Synthetic polypeptide
QMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARAPQWELVAEAFDMWGQGTMVTVSS

SEQ ID NO: 21 - protein, 108 aa, synthetic construct
  REGION 1..108: note = Description of Artificial Sequence: Synthetic polypeptide
SYVLTQPPSVSVAPGQTARITCGGNLIGSKLVHWYQQKPGQAPVLVVYDDSDRPSKIPERFSGSNSGNTATLTISRVEAGDEADYYCQVWDSSSDHVVFGGGTKLTVL

SEQ ID NO: 22 - protein, 122 aa, synthetic construct; identical to SEQ ID NO: 6
  REGION 1..122: note = Description of Artificial Sequence: Synthetic polypeptide

SEQ ID NO: 23 - protein, 108 aa, synthetic construct
  REGION 1..108: note = Description of Artificial Sequence: Synthetic polypeptide
SYVLTQPPSVSVAPGQTARITCGGNLIGSKLVHWYQQKPGQAPVLVVYDDSDRPSDIPERFSGSNSGNTATLTISRVEAGDEADYYCQVWDTSSDHVVFGGGTKLTVL

SEQ ID NO: 24 - protein, 122 aa, synthetic construct
  REGION 1..122: note = Description of Artificial Sequence: Synthetic polypeptide
QMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARAPQWEYVAEAFDLWGQGTMVTVSS

SEQ ID NO: 25 - protein, 108 aa, synthetic construct
  REGION 1..108: note = Description of Artificial Sequence: Synthetic polypeptide
SYVLTQPPSVSVAPGQTARITCGGNIIGSKLVHWYQQKPGQAPVLVVYDDGDRPSGIPERFSGSNSGNTATLTISRVEAGDEADYYCQVWDTSSDHVVFGGGTKLTVL

SEQ ID NO: 26 - protein, 122 aa, synthetic construct
  REGION 1..122: note = Description of Artificial Sequence: Synthetic polypeptide
QMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARAPQWELTAEAFDLWGQGTMVTVSS

SEQ ID NO: 27 - protein, 108 aa, synthetic construct
  REGION 1..108: note = Description of Artificial Sequence: Synthetic polypeptide
SYVLTQPPSVSVAPGQTARITCGGNLLGSKLVHWYQQKPGQAPVLVVYDDGDRPSLIPERFSGSNSGNTATLTISRVEAGDEADYYCQVWDTSSDHVVFGGGTKLTVL

SEQ ID NO: 28 - protein, 122 aa, synthetic construct; identical to SEQ ID NO: 6
  REGION 1..122: note = Description of Artificial Sequence: Synthetic polypeptide

SEQ ID NO: 29 - protein, 108 aa, synthetic construct
  REGION 1..108: note = Description of Artificial Sequence: Synthetic polypeptide
SYVLTQPPSVSVAPGQTARITCGGNLLGSKLVHWYQQKPGQAPVLVVYDDSDRPSEIPERFSGSNSGNTATLTISRVEAGDEADYYCQVWDTSSDGVVFGGGTKLTVL

SEQ ID NO: 30 - protein, 122 aa, synthetic construct; identical to SEQ ID NO: 4
  REGION 1..122: note = Description of Artificial Sequence: Synthetic polypeptide

SEQ ID NO: 31 - protein, 108 aa, synthetic construct
  REGION 1..108: note = Description of Artificial Sequence: Synthetic polypeptide
SYVLTQPPSVSVAPGQTARITCGGNLLGSKLVHWYQQKPGQAPVLVVYDDSDRPSWIPERFSGSNSGNTATLTISRVEAGDEADYYCQVWDTSSDHVVFGGGTKLTVL

SEQ ID NO: 32 - protein, 122 aa, synthetic construct; identical to SEQ ID NO: 26
  REGION 1..122: note = Description of Artificial Sequence: Synthetic polypeptide

SEQ ID NO: 33 - protein, 108 aa, synthetic construct
  REGION 1..108: note = Description of Artificial Sequence: Synthetic polypeptide
SYVLTQPPSVSVAPGQTARITCGGNLIGSKLVHWYQQKPGQAPVLVVYDDSDRPSAIPERFSGSNSGNTATLTISRVEAGDEADYYCQVWDTSSDHVVFGGGTKLTVL

SEQ ID NO: 34 - protein, 122 aa, synthetic construct
  REGION 1..122: note = Description of Artificial Sequence: Synthetic polypeptide
QMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARAPQWELTSEAFDLWGQGTMVTVSS

SEQ ID NO: 35 - protein, 108 aa, synthetic construct
  REGION 1..108: note = Description of Artificial Sequence: Synthetic polypeptide
SYVLTQPPSVSVAPGQTARITCGGNLIGSKLVHWYQQKPGQAPVLVVYDDSDRPSEIPERFSGSNSGNTATLTISRVEAGDEADYYCQVWDSSSDGVVFGGGTKLTVL

SEQ ID NO: 36 - protein, 122 aa, synthetic construct; identical to SEQ ID NO: 34
  REGION 1..122: note = Description of Artificial Sequence: Synthetic polypeptide

SEQ ID NO: 37 - protein, 108 aa, synthetic construct
  REGION 1..108: note = Description of Artificial Sequence: Synthetic polypeptide
SYVLTQPPSVSVAPGQTARITCGGNIIGSKLVHWYQQKPGQAPVLVVYDDSDRPSGIPERFSGSNSGNTATLTISRVEAGDEADYYCQVWDSGSDHVVFGGGTKLTVL

SEQ ID NO: 38 - protein, 122 aa, synthetic construct
  REGION 1..122: note = Description of Artificial Sequence: Synthetic polypeptide
QMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARAPQWLLVAEAFDLWGQGTMVTVSS

SEQ ID NO: 39 - protein, 108 aa, synthetic construct
  REGION 1..108: note = Description of Artificial Sequence: Synthetic polypeptide
SYVLTQPPSVSVAPGQTARITCGGNIIGSKLVHWYQQKPGQAPVLVVYDDSDRPSDIPERFSGSNSGNTATLTISRVEAGDEADYYCQVWDSGSDGVVFGGGTKLTVL

SEQ ID NO: 40 - protein, 122 aa, synthetic construct; identical to SEQ ID NO: 6
  REGION 1..122: note = Description of Artificial Sequence: Synthetic polypeptide

SEQ ID NO: 41 - protein, 108 aa, synthetic construct
  REGION 1..108: note = Description of Artificial Sequence: Synthetic polypeptide
SYVLTQPPSVSVAPGQTARITCGGNLIGSKLVHWYQQKPGQAPVLVVYDDGDRPSWIPERFSGSNSGNTATLTISRVEAGDEADYYCQVWDSGSDGVVFGGGTKLTVL

SEQ ID NO: 42 - protein, 122 aa, synthetic construct; identical to SEQ ID NO: 6
  REGION 1..122: note = Description of Artificial Sequence: Synthetic polypeptide

SEQ ID NO: 43 - protein, 108 aa, synthetic construct
  REGION 1..108: note = Description of Artificial Sequence: Synthetic polypeptide
SYVLTQPPSVSVAPGQTARITCGGNLIGSKLVHWYQQKPGQAPVLVVYDDSDRPSRIPERFSGSNSGNTATLTISRVEAGDEADYYCQVWDSGSDGVVFGGGTKLTVL

SEQ ID NO: 44 - protein, 122 aa, synthetic construct
  REGION 1..122: note = Description of Artificial Sequence: Synthetic polypeptide
QMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARAPQWVLVSEAFDLWGQGTMVTVSS

SEQ ID NO: 45 - protein, 108 aa, synthetic construct
  REGION 1..108: note = Description of Artificial Sequence: Synthetic polypeptide
SYVLTQPPSVSVAPGQTARITCGGNIIGSKLVHWYQQKPGQAPVLVVYDDSDRPSAIPERFSGSNSGNTATLTISRVEAGDEADYYCQVWDSSSDGVVFGGGTKLTVL

SEQ ID NO: 46 - protein, 122 aa, synthetic construct; identical to SEQ ID NO: 6
  REGION 1..122: note = Description of Artificial Sequence: Synthetic polypeptide

SEQ ID NO: 47 - protein, 108 aa, synthetic construct
  REGION 1..108: note = Description of Artificial Sequence: Synthetic polypeptide
SYVLTQPPSVSVAPGQTARITCGGNNIGSKLVHWYQQKPGQAPVLVVYDDSDRPSGIPERFSGSNSGNTATLTISRVEAGDEADYYCQVWDSSSDGVVFGGGTKLTVL

SEQ ID NO: 48 - protein, 122 aa, synthetic construct
  REGION 1..122: note = Description of Artificial Sequence: Synthetic polypeptide
QMQLVESGGGVVQPGRSLRLSCAASGFTFRTYGMHWVRQAPGKGLEWVAVISYDGSNKHYADSVKGRFTITRDNSKNTLNLQMNSLRAEDTAVYYCARSPQWEWVHEAFDMWGQGTMVTVSS

SEQ ID NO: 49 - protein, 108 aa, synthetic construct
  REGION 1..108: note = Description of Artificial Sequence: Synthetic polypeptide
SYVLTQPPSVSVAPGQTARITCGGNILGSKLVHWYQQKPGQAPVLVVYDDSDRPSEIPERFSGSNSGNTATLTISRVEAGDEADYYCQVWDTSSDGVVFGGGTKLTVL

SEQ ID NO: 50 - protein, 122 aa, synthetic construct
  REGION 1..122: note = Description of Artificial Sequence: Synthetic polypeptide
QMQLVESGGGVVQPGRSLRLSCAASGFTFRTYGMHWVRQAPGKGLEWVAVISYDGSNKHYADSVKGRFTITRDNSKNTLNLQMNSLRAEDTAVYYCARSPQWEWVHEAFDLWGQGTMVTVSS

SEQ ID NO: 51 - protein, 108 aa, synthetic construct
  REGION 1..108: note = Description of Artificial Sequence: Synthetic polypeptide
SYVLTQPPSVSVAPGQTARITCGGNNIGSKLVHWYQQKPGQAPVLVVYDDSDRPSRIPERFSGSNSGNTATLTISRVEAGDEADYYCQVWDTSSDGVVFGGGTKLTVL

SEQ ID NO: 52 - protein, 122 aa, synthetic construct
  REGION 1..122: note = Description of Artificial Sequence: Synthetic polypeptide
QMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARSPQWEWVHEAFDLWGQGTMVTVSS

SEQ ID NO: 53 - protein, 108 aa, synthetic construct
  REGION 1..108: note = Description of Artificial Sequence: Synthetic polypeptide
SYVLTQPPSVSVAPGQTARITCGGNLIGSKLVHWYQQKPGQAPVLVVYDDGDRPSWIPERFSGSNSGNTATLTISRVEAGDEADYYCQVWDSSSDGVVFGGGTKLTVL

SEQ ID NO: 54 - protein, 122 aa, synthetic construct; identical to SEQ ID NO: 52
  REGION 1..122: note = Description of Artificial Sequence: Synthetic polypeptide

SEQ ID NO: 55 - DNA, 366 bp, synthetic construct
  misc_feature 1..366: note = Description of Artificial Sequence: Synthetic polynucleotide
  source 1..366: mol_type = other DNA
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgcagtttattactgtgcccgtgcaccacagtgggaattagtacacgaagcattcgatatctggggtcagggtactatggtgaccgttagctct

SEQ ID NO: 56 - DNA, 324 bp, synthetic construct
  misc_feature 1..324: note = Description of Artificial Sequence: Synthetic polynucleotide
  source 1..324: mol_type = other DNA
tcttacgtgctgactcaaccaccatcagtgtctgtagcaccaggccagaccgcacgtattacctgtggcggtaacaatctgggctctaagtctgttcactggtatcagcaaaaaccaggccaggcaccagtactggttgtgtacgatgattccgatcgtccaagctggattccagagcgtttcagcggctctaattccggcaacaccgctactctgactatttcccgtggggaagccggcgatgaagccgactactattgccaggtctgggactcttcttccgaccatgtagtctttggcgggggcaccaaactgaccgttttg

SEQ ID NO: 57 - DNA, 366 bp, synthetic construct
  misc_feature 1..366: note = Description of Artificial Sequence: Synthetic polynucleotide
  source 1..366: mol_type = other DNA
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtgggaattaacggcggaagcattcgatatttggggccagggcactatggtgaccgttagctct

SEQ ID NO: 58 - DNA, 324 bp, synthetic construct
  misc_feature 1..324: note = Description of Artificial Sequence: Synthetic polynucleotide
  source 1..324: mol_type = other DNA
tcttacgtgctgactcaaccaccatcagtgtctgtagcaccaggccagaccgcacgtattacctgtggcggtaacctgatcggctctaagctggttcactggtatcagcaaaaaccaggccaggcaccagtactggttgtgtacgatgatagcgatcgtccaagcctgattccagagcgtttcagcggctctaattccggcaacaccgctactctgactatttcccgtgttgaagccggcgatgaagccgactactattgccaggtctgggactctagctccgacggtgtagtctttggcgggggcaccaaactgaccgttttg

SEQ ID NO: 59 - DNA, 366 bp, synthetic construct
  misc_feature 1..366: note = Description of Artificial Sequence: Synthetic polynucleotide
  source 1..366: mol_type = other DNA
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtgggaattagtagcggaagcattcgatctgtggggccagggcactatggtgaccgttagctct

SEQ ID NO: 60 - DNA, 324 bp, synthetic construct; identical to SEQ ID NO: 58
  misc_feature 1..324: note = Description of Artificial Sequence: Synthetic polynucleotide
  source 1..324: mol_type = other DNA

SEQ ID NO: 61 - DNA, 366 bp, synthetic construct
  misc_feature 1..366: note = Description of Artificial Sequence: Synthetic polynucleotide
  source 1..366: mol_type = other DNA
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtggcagttagtagcggaagcattcgatatttggggccagggcactatggtgaccgttagctct

SEQ ID NO: 62 - DNA, 324 bp, synthetic construct
  misc_feature 1..324: note = Description of Artificial Sequence: Synthetic polynucleotide
  source 1..324: mol_type = other DNA
tcttacgtgctgactcaaccaccatcagtgtctgtagcaccaggccagaccgcacgtattacctgtggcggtaacctgatcggctctaagctggttcactggtatcagcaaaaaccaggccaggcaccagtactggttgtgtacgatgatagcgatcgtccaagccttattccagagcgtttcagcggctctaattccggcaacaccgctactctgactatttcccgtgttgaagccggcgatgaagccgactactattgccaggtctgggacaccagctccgacggtgtagtctttggcgggggcaccaaactgaccgttttg

SEQ ID NO: 63 - DNA, 366 bp, synthetic construct
  misc_feature 1..366: note = Description of Artificial Sequence: Synthetic polynucleotide
  source 1..366: mol_type = other DNA
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtggcagttagtagccgaagcattcgatatatggggccagggcactatggtgaccgttagctct

SEQ ID NO: 64 - DNA, 324 bp, synthetic construct
  misc_feature 1..324: note = Description of Artificial Sequence: Synthetic polynucleotide
  source 1..324: mol_type = other DNA
tcttacgtgctgactcaaccaccatcagtgtctgtagcaccaggccagaccgcacgtattacctgtggcggtaacctgatcggctctaagctggttcactggtatcagcaaaaaccaggccaggcaccagtactggttgtgtacgatgatagcgatcgtccaagcgctattccagagcgtttcagcggctctaattccggcaacaccgctactctgactatttcccgtgttgaagccggcgatgaagccgactactattgccaggtctgggacactagctccgacggtgtagtctttggcgggggcaccaaactgaccgttttg

SEQ ID NO: 65 - DNA, 366 bp, synthetic construct
  misc_feature 1..366: note = Description of Artificial Sequence: Synthetic polynucleotide
  source 1..366: mol_type = other DNA
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtgggaattagtggcggaagcattcgatctttggggccagggcactatggtgaccgttagctct

SEQ ID NO: 66 - DNA, 324 bp, synthetic construct; identical to SEQ ID NO: 62
  misc_feature 1..324: note = Description of Artificial Sequence: Synthetic polynucleotide
  source 1..324: mol_type = other DNA

SEQ ID NO: 67 - DNA, 366 bp, synthetic construct
  misc_feature 1..366: note = Description of Artificial Sequence: Synthetic polynucleotide
  source 1..366: mol_type = other DNA
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtgggaattggtagccgaagcattcgatatctggggccagggcactatggtgaccgttagctct

SEQ ID NO: 68 - DNA, 324 bp, synthetic construct
  misc_feature 1..324: note = Description of Artificial Sequence: Synthetic polynucleotide
  source 1..324: mol_type = other DNA
tcttacgtgctgactcaaccaccatcagtgtctgtagcaccaggccagaccgcacgtattacctgtggcggtaacctgctgggctctaagctggttcactggtatcagcaaaaaccaggccaggcaccagtactggttgtgtacgatgatagcgatcgtccaagcgcaattccagagcgtttcagcggctctaattccggcaacaccgctactctgactatttcccgtgttgaagccggcgatgaagccgactactattgccaggtctgggactctagctccgaccacgtagtctttggcgggggcaccaaactgaccgttttg

SEQ ID NO: 69 - DNA, 366 bp, synthetic construct
  misc_feature 1..366: note = Description of Artificial Sequence: Synthetic polynucleotide
  source 1..366: mol_type = other DNA
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtggatgttagtagcggaagcattcgatctatggggccagggcactatggtgaccgttagctct

SEQ ID NO: 70 - DNA, 324 bp, synthetic construct
  misc_feature 1..324: note = Description of Artificial Sequence: Synthetic polynucleotide
  source 1..324: mol_type = other DNA
tcttacgtgctgactcaaccaccatcagtgtctgtagcaccaggccagaccgcacgtattacctgtggcggtaacctgatcggctctaagctggttcactggtatcagcaaaaaccagggcaggcaccagtactggttgtgtacgatgatagcgatcgtccaagccggattccagagcgtttcagcggctctaattccggcaacaccgctactctgactatttcccgtgttgaagccggcgatgaagccgactactattgccaggtctgggactccagctccgaccatgtagtctttggcgggggcaccaaactgaccgttttg

SEQ ID NO: 71 - DNA, 366 bp, synthetic construct
  misc_feature 1..366: note = Description of Artificial Sequence: Synthetic polynucleotide
  source 1..366: mol_type = other DNA
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtgggaatgggtagccgaagcattcgatctgtggggccagggcactatggtgaccgttagctct

SEQ ID NO: 72 - DNA, 324 bp, synthetic construct
  misc_feature 1..324: note = Description of Artificial Sequence: Synthetic polynucleotide
  source 1..324: mol_type = other DNA
tcttacgtgctgactcaaccaccatcagtgtctgtagcaccaggccagaccgcacgtattacctgtggcggtaacctgatcggctctaagctggttcactggtatcagcaaaaaccaggccaggcaccagtactggttgtgtacgatgatagcgatcgtccaagccggattccagagcgtttcagcggctctaattccggcaacaccgctactctgactatttcccgtgttgaagccggcgatgaagccgactactattgccaggtctgggactccagctccgaccacgtagtctttggcgggggcaccaaactgaccgttttg

SEQ ID NO: 73 - DNA, 366 bp, synthetic construct
  misc_feature 1..366: note = Description of Artificial Sequence: Synthetic polynucleotide
  source 1..366: mol_type = other DNA
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtgggaattagtagccgaggcattcgatatgtggggccagggcactatggtgaccgttagctct

SEQ ID NO: 74 - DNA, 324 bp, synthetic construct
  misc_feature 1..324: note = Description of Artificial Sequence: Synthetic polynucleotide
  source 1..324: mol_type = other DNA
tcttacgtgctgactcaaccaccatcagtgtctgtagcaccaggccagaccgcacgtattacctgtggcggtaacctgatcggctctaagctggttcactggtatcagcaaaaaccaggccaggcaccagtactggttgtgtacgatgatagcgatcgtccaagccggattccagagcgtttcagcggctctaattccggcaacaccgctactctgactatttcccgtgttgaagccggcgatgaagccgactactattgccaggtctgggactctagctccgaccatgtagtctttggcgggggcaccaaactgaccgttttg

SEQ ID NO: 75 - DNA, 366 bp, synthetic construct
  misc_feature 1..366: note = Description of Artificial Sequence: Synthetic polynucleotide
  source 1..366: mol_type = other DNA
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtgggaattagtagccgaagcattcgatctgtggggccagggcactatggtgaccgttagctct

SEQ ID NO: 76 - DNA, 324 bp, synthetic construct
  misc_feature 1..324: note = Description of Artificial Sequence: Synthetic polynucleotide
  source 1..324: mol_type = other DNA
tcttacgtgctgactcaaccaccatcagtgtctgtagcaccaggccagaccgcacgtattacctgtggcggtaacctgatcggctctaagctggttcactggtatcagcaaaaaccaggccaggcaccagtactggttgtgtacgatgatagcgatcgtccaagcaaaattccagagcgtttcagcggctctaattccggcaacaccgctactctgactatttcccgtgttgaagccggcgatgaagccgactactattgccaggtctgggactccagctccgaccatgtagtctttggcgggggcaccaaactgaccgttttg

SEQ ID NO: 77 - DNA, 366 bp, synthetic construct
  misc_feature 1..366: note = Description of Artificial Sequence: Synthetic polynucleotide
  source 1..366: mol_type = other DNA
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtgggaatacgtagcggaagcattcgatctgtggggccagggcactatggtgaccgttagctct

SEQ ID NO: 78 - DNA, 324 bp, synthetic construct
  misc_feature 1..324: note = Description of Artificial Sequence: Synthetic polynucleotide
  source 1..324: mol_type = other DNA
tcttacgtgctgactcaaccaccatcagtgtctgtagcaccaggccagaccgcacgtattacctgtggcggtaacctgatcggctctaagctggttcactggtatcagcaaaaaccaggccaggcaccagtactggttgtgtacgatgatagcgatcgtccaagcgatattccagagcgtttcagcggctctaattccggcaacaccgctactctgactatttcccgtgttgaagccggcgatgaagccgactactattgccaggtctgggacaccagctccgaccatgtagtctttggcgggggcaccaaactgaccgttttg

SEQ ID NO: 79 - DNA, 366 bp, synthetic construct
  misc_feature 1..366: note = Description of Artificial Sequence: Synthetic polynucleotide
  source 1..366: mol_type = other DNA
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtgggaattaacggccgaagcattcgatctttggggccagggcactatggtgaccgttagctct

SEQ ID NO: 80 - DNA, 324 bp, synthetic construct
  misc_feature 1..324: note = Description of Artificial Sequence: Synthetic polynucleotide
  source 1..324: mol_type = other DNA
tcttacgtgctgactcaaccaccatcagtgtctgtagcaccaggccagaccgcacgtattacctgtggcggtaacatcatcggctctaagctggttcactggtatcagcaaaaaccaggccaggcaccagtactggttgtgtacgatgatggcgatcgtccaagcggtattccagagcgtttcagcggctctaattccggcaacaccgctactctgactatttcccgtgttgaagccggcgatgaagccgactactattgccaggtctgggacactagctccgaccacgtagtctttggcgggggcaccaaactgaccgttttg

SEQ ID NO: 81 - DNA, 366 bp, synthetic construct; identical to SEQ ID NO: 65
  misc_feature 1..366: note = Description of Artificial Sequence: Synthetic polynucleotide
  source 1..366: mol_type = other DNA

SEQ ID NO: 82 - DNA, 324 bp, synthetic construct
  misc_feature 1..324: note = Description of Artificial Sequence: Synthetic polynucleotide
  source 1..324: mol_type = other DNA
tcttacgtgctgactcaaccaccatcagtgtctgtagcaccaggccagaccgcacgtattacctgtggcggtaacctgctgggctctaagctggttcactggtatcagcaaaaaccaggccaggcaccagtactggttgtgtacgatgatggcgatcgtccaagcctgattccagagcgtttcagcggctctaattccggcaacaccgctactctgactatttcccgtgttgaagccggcgatgaagccgactactattgccaggtctgggacactagctccgaccacgtagtctttggcgggggcaccaaactgaccgttttg

SEQ ID NO: 83 - DNA, 366 bp, synthetic construct; identical to SEQ ID NO: 57
  misc_feature 1..366: note = Description of Artificial Sequence: Synthetic polynucleotide
  source 1..366: mol_type = other DNA

SEQ ID NO: 84 - DNA, 324 bp, synthetic construct
  misc_feature 1..324: note = Description of Artificial Sequence: Synthetic polynucleotide
  source 1..324: mol_type = other DNA
tcttacgtgctgactcaaccaccatcagtgtctgtagcaccaggccagaccgcacgtattacctgtggcggtaacctgctgggctctaagctggttcactggtatcagcaaaaaccaggccaggcaccagtactggttgtgtacgatgatagcgatcgtccaagcgaaattccagagcgtttcagcggctctaattccggcaacaccgctactctgactatttcccgtgttgaagccggcgatgaagccgactactattgccaggtctgggacaccagctccgacggtgtagtctttggcgggggcaccaaactgaccgttttg

SEQ ID NO: 85 - DNA, 366 bp, synthetic construct; identical to SEQ ID NO: 79
  misc_feature 1..366: note = Description of Artificial Sequence: Synthetic polynucleotide
  source 1..366: mol_type = other DNA

SEQ ID NO: 86 - DNA, 324 bp, synthetic construct
  misc_feature 1..324: note = Description of Artificial Sequence: Synthetic polynucleotide
  source 1..324: mol_type = other DNA
tcttacgtgctgactcaaccaccatcagtgtctgtagcaccaggccagaccgcacgtattacctgtggcggtaacctgctgggctctaagctggttcactggtatcagcaaaaaccaggccaggcaccagtactggttgtgtacgatgatagcgatcgtccaagctggattccagagcgtttcagcggctctaattccggcaacaccgctactctgactatttcccgtgttgaagccggcgatgaagccgactactattgccaggtctgggacactagctccgaccatgtagtctttggcgggggcaccaaactgaccgttttg

SEQ ID NO: 87 - DNA, 366 bp, synthetic construct
  misc_feature 1..366: note = Description of Artificial Sequence: Synthetic polynucleotide
  source 1..366: mol_type = other DNA
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtgggaattaacgtcggaagcattcgatctttggggccagggcactatggtgaccgttagctct

SEQ ID NO: 88 - DNA, 324 bp, synthetic construct
  misc_feature 1..324: note = Description of Artificial Sequence: Synthetic polynucleotide
  source 1..324: mol_type = other DNA
tcttacgtgctgactcaaccaccatcagtgtctgtagcaccaggccagaccgcacgtattacctgtggcggtaacctgatcggctctaagctggttcactggtatcagcaaaaaccaggccaggcaccagtactggttgtgtacgatgatagcgatcgtccaagcgcaattccagagcgtttcagcggctctaattccggcaacaccgctactctgactatttcccgtgttgaagccggcgatgaagccgactactattgccaggtctgggacaccagctccgaccacgtagtctttggcgggggcaccaaactgaccgttttg

SEQ ID NO: 89 - DNA, 366 bp, synthetic construct
  misc_feature 1..366: note = Description of Artificial Sequence: Synthetic polynucleotide
  source 1..366: mol_type = other DNA
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtgggaattaacctccgaagcattcgatctttggggccagggcactatggtgaccgttagctct

SEQ ID NO: 90 - DNA, 324 bp, synthetic construct
  misc_feature 1..324: note = Description of Artificial Sequence: Synthetic polynucleotide
  source 1..324: mol_type = other DNA
tcttacgtgctgactcaaccaccatcagtgtctgtagcaccaggccagaccgcacgtattacctgtggcggtaacctgatcggctctaagctggttcactggtatcagcaaaaaccaggccaggcaccagtactggttgtgtacgatgatagcgatcgtccaagcgaaattccagagcgtttcagcggctctaattccggcaacaccgctactctgactatttcccgtgttgaagccggcgatgaagccgactactattgccaggtctgggactccagctccgacggtgtagtctttggcgggggcaccaaactgaccgttttg

SEQ ID NO: 91 - DNA, 366 bp, synthetic construct
  misc_feature 1..366: note = Description of Artificial Sequence: Synthetic polynucleotide
  source 1..366: mol_type = other DNA
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtggctgttagtagcggaagcattcgatctctggggccagggcactatggtgaccgttagctct

SEQ ID NO: 92 - DNA, 324 bp, synthetic construct
  misc_feature 1..324: note = Description of Artificial Sequence: Synthetic polynucleotide
  source 1..324: mol_type = other DNA
tcttacgtgctgactcaaccaccatcagtgtctgtagcaccaggccagaccgcacgtattacctgtggcggtaacatcatcggctctaagctggttcactggtatcagcaaaaaccaggccaggcaccagtactggttgtgtacgatgatagcgatcgtccaagcggtattccagagcgtttcagcggctctaattccggcaacaccgctactctgactatttcccgtgttgaagccggcgatgaagccgactactattgccaggtctgggactctggctccgaccacgtagtctttggcgggggcaccaaactgaccgttttg

SEQ ID NO: 93 - DNA, 366 bp, synthetic construct
  misc_feature 1..366: note = Description of Artificial Sequence: Synthetic polynucleotide
  source 1..366: mol_type = other DNA
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtgggaattagtagcggaagcattcgatctttggggccagggcactatggtgaccgttagctct

SEQ ID NO: 94 - DNA, 324 bp, synthetic construct
  misc_feature 1..324: note = Description of Artificial Sequence: Synthetic polynucleotide
  source 1..324: mol_type = other DNA
tcttacgtgctgactcaaccaccatcagtgtctgtagcaccaggccagaccgcacgtattacctgtggcggtaacatcatcggctctaagctggttcactggtatcagcaaaaaccaggccaggcaccagtactggttgtgtacgatgatagcgatcgtccaagcgatattccagagcgtttcagcggctctaattccggcaacaccgctactctgactatttcccgtgttgaagccggcgatgaagccgactactattgccaggtctgggactccggctccgacggtgtagtctttggcgggggcaccaaactgaccgttttg

SEQ ID NO: 95 - DNA, 366 bp, synthetic construct; identical to SEQ ID NO: 59
  misc_feature 1..366: note = Description of Artificial Sequence: Synthetic polynucleotide
  source 1..366: mol_type = other DNA

SEQ ID NO: 96 - DNA, 324 bp, synthetic construct
  misc_feature 1..324: note = Description of Artificial Sequence: Synthetic polynucleotide
  source 1..324: mol_type = other DNA
tcttacgtgctgactcaaccaccatcagtgtctgtagcaccaggccagaccgcacgtattacctgtggcggtaacctgatcggctctaagctggttcactggtatcagcaaaaaccaggccaggcaccagtactggttgtgtacgatgatggcgatcgtccaagctggattccagagcgtttcagcggctctaattccggcaacaccgctactctgactatttcccgtgttgaagccggcgatgaagccgactactattgccaggtctgggactctggctccgacggtgtagtctttggcgggggcaccaaactgaccgttttg

SEQ ID NO: 97 - DNA, 366 bp, synthetic construct
  misc_feature 1..366: note = Description of Artificial Sequence: Synthetic polynucleotide
  source 1..366: mol_type = other DNA
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtgggtcttagtatccgaagcattcgatctttggggccagggcactatggtgaccgttagctct

SEQ ID NO: 98 - DNA, 324 bp, synthetic construct
  misc_feature 1..324: note = Description of Artificial Sequence: Synthetic polynucleotide
  source 1..324: mol_type = other DNA
tcttacgtgctgactcaaccaccatcagtgtctgtagcaccaggccagaccgcacgtattacctgtggcggtaacctgatcggctctaagctggttcactggtatcagcaaaaaccaggccaggcaccagtactggttgtgtacgatgatagcgatcgtccaagccgtattccagagcgtttcagcggctctaattccggcaacaccgctactctgactatttcccgtgttgaagccggcgatgaagccgactactattgccaggtctgggactccggctccgacggtgtagtctttggcgggggcaccaaactgaccgttttg

SEQ ID NO: 99 - DNA, 366 bp, synthetic construct
  misc_feature 1..366: note = Description of Artificial Sequence: Synthetic polynucleotide
  source 1..366: mol_type = other DNA
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtgggaattagtagcggaggcattcgatctgtggggccagggcactatggtgaccgttagctct

SEQ ID NO: 100 - DNA, 324 bp, synthetic construct
  misc_feature 1..324: note = Description of Artificial Sequence: Synthetic polynucleotide
  source 1..324: mol_type = other DNA
tcttacgtgctgactcaaccaccatcagtgtctgtagcaccaggccagaccgcacgtattacctgtggcggtaacatcatcggctctaagctggttcactggtatcagcaaaaaccaggccaggcaccagtactggttgtgtacgatgatagcgatcgtccaagcgcaattccagagcgtttcagcggctctaattccggcaacaccgctactctgactatttcccgtgttgaagccggcgatgaagccgactactattgccaggtctgggactccagctccgacggtgtagtctttggcgggggcaccaaactgaccgttttg

SEQ ID NO: 101 - DNA, 366 bp, synthetic construct
  misc_feature 1..366: note = Description of Artificial Sequence: Synthetic polynucleotide
  source 1..366: mol_type = other DNA
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctcttatgatggtagcaataaacactatgctgactcagtcaaaggccgtttcaccatcacccgtgataacagcaagaacactcttaacttacagatgaactctctgcgtgccgaagacaccgccgtttactactgtgcccgtagcccacagtgggaatgggtacacgaagcattcgatatgtggggccagggcactatggtgaccgttagctct

SEQ ID NO: 102 - DNA, 324 bp, synthetic construct
  misc_feature 1..324: note = Description of Artificial Sequence: Synthetic polynucleotide
  source 1..324: mol_type = other DNA
tcttacgtgctgactcaaccaccatcagtgtctgtagcaccaggccagaccgcacgtattacctgtggcggtaacaacatcggctctaagctggttcactggtatcagcaaaaaccaggccaggcaccagtactggttgtgtacgatgatagcgatcgtccaagcggtattccagagcgtttcagcggctctaattccggcaacaccgctactctgactatttcccgtgttgaagccggcgatgaagccgactactattgccaggtctgggactctagctccgacggtgtagtctttggcgggggcaccaaactgaccgttttg

SEQ ID NO: 103 - DNA, 366 bp, synthetic construct
  misc_feature 1..366: note = Description of Artificial Sequence: Synthetic polynucleotide
  source 1..366: mol_type = other DNA
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctcttatgatggtagcaataaacactatgctgactcagtcaaaggccgtttcaccatcacccgtgataacagcaagaacactcttaacttacagatgaactctctgcgtgccgaagacaccgccgtttactactgtgcccgttcgccacagtgggaatgggtacacgaagcattcgatctctggggccagggcactatggtgaccgttagctct

SEQ ID NO: 104 - DNA, 324 bp, synthetic construct
  misc_feature 1..324: note = Description of Artificial Sequence: Synthetic polynucleotide
  source 1..324: mol_type = other DNA
tcttacgtgctgactcaaccaccatcagtgtctgtagcaccaggccagaccgcacgtattacctgtggcggtaacatcctgggctctaagctggttcactggtatcagcaaaaaccaggccaggcaccagtactggttgtgtacgatgatagcgatcgtccaagcgaaattccagagcgtttcagcggctctaattccggcaacaccgctactctgactatttcccgtgttgaagccggcgatgaagccgactactattgccaggtctgggacaccagctccgacggtgtagtctttggcgggggcaccaaactgaccgttttg

SEQ ID NO: 105 - DNA, 366 bp, synthetic construct
  misc_feature 1..366: note = Description of Artificial Sequence: Synthetic polynucleotide
  source 1..366: mol_type = other DNA
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtagcccacagtgggaatgggtacacgaagcattcgatctatggggccagggcactatggtgaccgttagctct

SEQ ID NO: 106 - DNA, 324 bp, synthetic construct
  misc_feature 1..324: note = Description of Artificial Sequence: Synthetic polynucleotide
  source 1..324: mol_type = other DNA
tcttacgtgctgactcaaccaccatcagtgtctgtagcaccaggccagaccgcacgtattacctgtggcggtaacaacatcggctctaagctggttcactggtatcagcaaaaaccaggccaggcaccagtactggttgtgtacgatgatagcgatcgtccaagccggattccagagcgtttcagcggctctaattccggcaacaccgctactctgactatttcccgtgttgaagccggcgatgaagccgactactattgccaggtctgggacactagctccgacggtgtagtctttggcgggggcaccaaactgaccgttttg

SEQ ID NO: 107 - DNA, 366 bp, synthetic construct
  misc_feature 1..366: note = Description of Artificial Sequence: Synthetic polynucleotide
  source 1..366: mol_type = other DNA
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgttcgccacagtgggaatgggtacacgaagcattcgatctctggggccagggcactatggtgaccgttagctct

SEQ ID NO: 108 - DNA, 324 bp, synthetic construct
  misc_feature 1..324: note = Description of Artificial Sequence: Synthetic polynucleotide
  source 1..324: mol_type = other DNA
tcttacgtgctgactcaaccaccatcagtgtctgtagcaccaggccagaccgcacgtattacctgtggcggtaacctgatcggctctaagctggttcactggtatcagcaaaaaccaggccaggcaccagtactggttgtgtacgatgatggcgatcgtccaagctggattccagagcgtttcagcggctctaattccggcaacaccgctactctgactatttcccgtgttgaagccggcgatgaagccgactactattgccaggtctgggactccagctccgacggtgtagtctttggcgggggcaccaaactgaccgttttg

SEQ ID NO: 109 - DNA, 735 bp, synthetic construct
  misc_feature 1..735: note = Description of Artificial Sequence: Synthetic polynucleotide
  source 1..735: mol_type = other DNA
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgcagtttattactgtgcccgtgcaccacagtgggaattagtacacgaagcattcgatatctggggtcagggtactatggtgaccgttagctctggcggtggtggtagcggaggcggaggatcaggtggaggcggcagttcttacgtgctgactcaaccaccatcagtgtctgtagcaccaggccagaccgcacgtattacctgtggcggtaacaatctgggctctaagtctgttcactggtatcagcaaaaaccaggccaggcaccagtactggttgtgtacgatgattccgatcgtccaagctggattccagagcgtttcagcggctctaattccggcaacaccgctactctgactatttcccgtggggaagccggcgatgaagccgactactattgccaggtctgggactcttcttccgaccatgtagtctttggcgggggcaccaaactgaccgttttg

SEQ ID NO: 110 - DNA, 735 bp, synthetic construct
  misc_feature 1..735: note = Description of Artificial Sequence: Synthetic polynucleotide
  source 1..735: mol_type = other DNA
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtgggaattaacggcggaagcattcgatatttggggccagggcactatggtgaccgttagctctggcggtggtggtagcggaggcggaggatcaggtggaggcggcagttcttacgtgctgactcaaccaccatcagtgtctgtagcaccaggccagaccgcacgtattacctgtggcggtaacctgatcggctctaagctggttcactggtatcagcaaaaaccaggccaggcaccagtactggttgtgtacgatgatagcgatcgtccaagcctgattccagagcgtttcagcggctctaattccggcaacaccgctactctgactatttcccgtgttgaagccggcgatgaagccgactactattgccaggtctgggactctagctccgacggtgtagtctttggcgggggcaccaaactgaccgttttg

SEQ ID NO: 111 - DNA, 735 bp, synthetic construct
  misc_feature 1..735: note = Description of Artificial Sequence: Synthetic polynucleotide
  source 1..735: mol_type = other DNA
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtgggaattagtagcggaagcattcgatctgtggggccagggcactatggtgaccgttagctctggcggtggtggtagcggaggcggaggatcaggtggaggcggcagttcttacgtgctgactcaaccaccatcagtgtctgtagcaccaggccagaccgcacgtattacctgtggcggtaacctgatcggctctaagctggttcactggtatcagcaaaaaccaggccaggcaccagtactggttgtgtacgatgatagcgatcgtccaagcctgattccagagcgtttcagcggctctaattccggcaacaccgctactctgactatttcccgtgttgaagccggcgatgaagccgactactattgccaggtctgggactctagctccgacggtgtagtctttggcgggggcaccaaactgaccgttttg

SEQ ID NO: 112 - DNA, 735 bp, synthetic construct
  misc_feature 1..735: note = Description of Artificial Sequence: Synthetic polynucleotide
  source 1..735: mol_type = other DNA
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtggcagttagtagcggaagcattcgatatttggggccagggcactatggtgaccgttagctctggcggtggtggtagcggaggcggaggatcaggtggaggcggcagttcttacgtgctgactcaaccaccatcagtgtctgtagcaccaggccagaccgcacgtattacctgtggcggtaacctgatcggctctaagctggttcactggtatcagcaaaaaccaggccaggcaccagtactggttgtgtacgatgatagcgatcgtccaagccttattccagagcgtttcagcggctctaattccggcaacaccgctactctgactatttcccgtgttgaagccggcgatgaagccgactactattgccaggtctgggacaccagctccgacggtgtagtctttggcgggggcaccaaactgaccgttttg

SEQ ID NO: 113 - DNA, 735 bp, synthetic construct
  misc_feature 1..735: note = Description of Artificial Sequence: Synthetic polynucleotide
  source 1..735: mol_type = other DNA
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtggcagttagtagccgaagcattcgatatatggggccagggcactatggtgaccgttagctctggcggtggtggtagcggaggcggaggatcaggtggaggcggcagttcttacgtgctgactcaaccaccatcagtgtctgtagcaccaggccagaccgcacgtattacctgtggcggtaacctgatcggctctaagctggttcactggtatcagcaaaaaccaggccaggcaccagtactggttgtgtacgatgatagcgatcgtccaagcgctattccagagcgtttcagcggctctaattccggcaacaccgctactctgactatttcccgtgttgaagccggcgatgaagccgactactattgccaggtctgggacactagctccgacggtgtagtctttggcgggggcaccaaactgaccgttttg

SEQ ID NO: 114 - DNA, 735 bp, synthetic construct
  misc_feature 1..735: note = Description of Artificial Sequence: Synthetic polynucleotide
  source 1..735: mol_type = other DNA
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtgggaattagtggcggaagcattcgatctttggggccagggcactatggtgaccgttagctctggtggtggtggtagcggaggcggaggatcaggtggaggcggcagttcttacgtgctgactcaaccaccatcagtgtctgtagcaccaggccagaccgcacgtattacctgtggcggtaacctgatcggctctaagctggttcactggtatcagcaaaaaccaggccaggcaccagtactggttgtgtacgatgatagcgatcgtccaagccttattccagagcgtttcagcggctctaattccggcaacaccgctactctgactatttcccgtgttgaagccggcgatgaagccgactactattgccaggtctgggacaccagctccgacggtgtagtctttggcgggggcaccaaactgaccgttttg

SEQ ID NO: 115 - DNA, 735 bp, synthetic construct
  misc_feature 1..735: note = Description of Artificial Sequence: Synthetic polynucleotide
  source 1..735: mol_type = other DNA
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtgggaattggtagccgaagcattcgatatctggggccagggcactatggtgaccgttagctctggcggtggtggtagcggaggcggaggatcaggtggaggcggcagttcttacgtgctgactcaaccaccatcagtgtctgtagcaccaggccagaccgcacgtattacctgtggcggtaacctgctgggctctaagctggttcactggtatcagcaaaaaccaggccaggcaccagtactggttgtgtacgatgatagcgatcgtccaagcgcaattccagagcgtttcagcggctctaattccggcaacaccgctactctgactatttcccgtgttgaagccggcgatgaagccgactactattgccaggtctgggactctagctccgaccacgtagtctttggcgggggcaccaaactgaccgttttg

SEQ ID NO: 116 - DNA, 735 bp, synthetic construct
  misc_feature 1..735: note = Description of Artificial Sequence: Synthetic polynucleotide
  source 1..735: mol_type = other DNA
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtggatgttagtagcggaagcattcgatctatggggccagggcactatggtgaccgttagctctggcggtggtggtagcggaggcggaggatcaggtggaggcggcagttcttacgtgctgactcaaccaccatcagtgtctgtagcaccaggccagaccgcacgtattacctgtggcggtaacctgatcggctctaagctggttcactggtatcagcaaaaaccagggcaggcaccagtactggttgtgtacgatgatagcgatcgtccaagccggattccagagcgtttcagcggctctaattccggcaacaccgctactctgactatttcccgtgttgaagccggcgatgaagccgactactattgccaggtctgggactccagctccgaccatgtagtctttggcgggggcaccaaactgaccgttttg

SEQ ID NO: 117 - DNA, 735 bp, synthetic construct
  misc_feature 1..735: note = Description of Artificial Sequence: Synthetic polynucleotide
  source 1..735: mol_type = other DNA
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtgggaatgggtagccgaagcattcgatctgtggggccagggcactatggtgaccgttagctctggcggtggtggtagcggaggcggaggatcaggtggaggcggcagttcttacgtgctgactcaaccaccatcagtgtctgtagcaccaggccagaccgcacgtattacctgtggcggtaacctgatcggctctaagctggttcactggtatcagcaaaaaccaggccaggcaccagtactggttgtgtacgatgatagcgatcgtccaagccggattccagagcgtttcagcggctctaattccggcaacaccgctactctgactatttcccgtgttgaagccggcgatgaagccgactactattgccaggtctgggactccagctccgaccacgtagtctttggcgggggcaccaaactgaccgttttg

SEQ ID NO: 118 - DNA, 735 bp, synthetic construct
  misc_feature 1..735: note = Description of Artificial Sequence: Synthetic polynucleotide
  source 1..735: mol_type = other DNA
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtgggaattagtagccgaggcattcgatatgtggggccagggcactatggtgaccgttagctctggcggtggtggtagcggaggcggaggatcaggtggaggcggcagttcttacgtgctgactcaaccaccatcagtgtctgtagcaccaggccagaccgcacgtattacctgtggcggtaacctgatcggctctaagctggttcactggtatcagcaaaaaccaggccaggcaccagtactggttgtgtacgatgatagcgatcgtccaagccggattccagagcgtttcagcggctctaattccggcaacaccgctactctgactatttcccgtgttgaagccggcgatgaagccgactactattgccaggtctgggactctagctccgaccatgtagtctttggcgggggcaccaaactgaccgttttg

SEQ ID NO: 119 - DNA, 735 bp, synthetic construct
  misc_feature 1..735: note = Description of Artificial Sequence: Synthetic polynucleotide
  source 1..735: mol_type = other DNA
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtgggaattagtagccgaagcattcgatctgtggggccagggcactatggtgaccgttagctctggcggtggtggtagcggaggcggaggatcaggtggaggcggcagttcttacgtgctgactcaaccaccatcagtgtctgtagcaccaggccagaccgcacgtattacctgtggcggtaacctgatcggctctaagctggttcactggtatcagcaaaaaccaggccaggcaccagtactggttgtgtacgatgatagcgatcgtccaagcaaaattccagagcgtttcagcggctctaattccggcaacaccgctactctgactatttcccgtgttgaagccggcgatgaagccgactactattgccaggtctgggactccagctccgaccatgtagtctttggcgggggcaccaaactgaccgttttg

SEQ ID NO: 120 - DNA, 735 bp, synthetic construct
  misc_feature 1..735: note = Description of Artificial Sequence: Synthetic polynucleotide
  source 1..735: mol_type = other DNA
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtgggaatacgtagcggaagcattcgatctgtggggccagggcactatggtgaccgttagctctggcggtggtggtagcggaggcggaggatcaggtggaggcggcagttcttacgtgctgactcaaccaccatcagtgtctgtagcaccaggccagaccgcacgtattacctgtggcggtaacctgatcggctctaagctggttcactggtatcagcaaaaaccaggccaggcaccagtactggttgtgtacgatgatagcgatcgtccaagcgatattccagagcgtttcagcggctctaattccggcaacaccgctactctgactatttcccgtgttgaagccggcgatgaagccgactactattgccaggtctgggacaccagctccgaccatgtagtctttggcgggggcaccaaactgaccgttttg

SEQ ID NO: 121 - DNA, 735 bp, synthetic construct
  misc_feature 1..735: note = Description of Artificial Sequence: Synthetic polynucleotide
  source 1..735: mol_type = other DNA
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtgggaattaacggccgaagcattcgatctttggggccagggcactatggtgaccgttagctctggcggtggtggtagcggaggcggaggatcaggtggaggcggcagttcttacgtgctgactcaaccaccatcagtgtctgtagcaccaggccagaccgcacgtattacctgtggcggtaacatcatcggctctaagctggttcactggtatcagcaaaaaccaggccaggcaccagtactggttgtgtacgatgatggcgatcgtccaagcggtattccagagcgtttcagcggctctaattccggcaacaccgctactctgactatttcccgtgttgaagccggcgatgaagccgactactattgccaggtctgggacactagctccgaccacgtagtctttggcgggggcaccaaactgaccgttttg

SEQ ID NO: 122 - DNA, 735 bp, synthetic construct
  misc_feature 1..735: note = Description of Artificial Sequence: Synthetic polynucleotide
  source 1..735: mol_type = other DNA
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtgggaattagtggcggaagcattcgatctttggggccagggcactatggtgaccgttagctctggcggtggtggtagcggaggcggaggatcaggtggaggcggcagttcttacgtgctgactcaaccaccatcagtgtctgtagcaccaggccagaccgcacgtattacctgtggcggtaacctgctgggctctaagctggttcactggtatcagcaaaaaccaggccaggcaccagtactggttgtgtacgatgatggcgatcgtccaagcctgattccagagcgtttcagcggctctaattccggcaacaccgctactctgactatttcccgtgttgaagccggcgatgaagccgactactattgccaggtctgggacactagctccgaccacgtagtctttggcgggggcaccaaactgaccgttttg

SEQ ID NO: 123 - DNA, 735 bp, synthetic construct
  misc_feature 1..735: note = Description of Artificial Sequence: Synthetic polynucleotide
  source 1..735: mol_type = other DNA
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtgggaattaacggcggaagcattcgatatttggggccagggcactatggtgaccgttagctctggcggtggtggtagcggaggcggaggatcaggtggaggcggcagttcttacgtgctgactcaaccaccatcagtgtctgtagcaccaggccagaccgcacgtattacctgtggcggtaacctgctgggctctaagctggttcactggtatcagcaaaaaccaggccaggcaccagtactggttgtgtacgatgatagcgatcgtccaagcgaaattccagagcgtttcagcggctctaattccggcaacaccgctactctgactatttcccgtgttgaagccggcgatgaagccgactactattgccaggtctgggacaccagctccgacggtgtagtctttggcgggggcaccaaactgaccgttttg

SEQ ID NO: 124 - DNA, 735 bp, synthetic construct
  misc_feature 1..735: note = Description of Artificial Sequence: Synthetic polynucleotide
  source 1..735: mol_type = other DNA
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtgggaattaacggccgaagcattcgatctttggggccagggcactatggtgaccgttagctctggcggtggtggtagcggaggcggaggatcaggtggaggcggcagttcttacgtgctgactcaaccaccatcagtgtctgtagcaccaggccagaccgcacgtattacctgtggcggtaacctgctgggctctaagctggttcactggtatcagcaaaaaccaggccaggcaccagtactggttgtgtacgatgatagcgatcgtccaagctggattccagagcgtttcagcggctctaattccggcaacaccgctactctgactatttcccgtgttgaagccggcgatgaagccgactactattgccaggtctgggacactagctccgaccatgtagtctttggcgggggcaccaaactgaccgttttg

SEQ ID NO: 125 - DNA, 735 bp, synthetic construct
  misc_feature 1..735: note = Description of Artificial Sequence: Synthetic polynucleotide
  source 1..735: mol_type = other DNA
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtgggaattaacgtcggaagcattcgatctttggggccagggcactatggtgaccgttagctctggcggtggtggtagcggaggcggaggatcaggtggaggcggcagttcttacgtgctgactcaaccaccatcagtgtctgtagcaccaggccagaccgcacgtattacctgtggcggtaacctgatcggctctaagctggttcactggtatcagcaaaaaccaggccaggcaccagtactggttgtgtacgatgatagcgatcgtccaagcgcaattccagagcgtttcagcggctctaattccggcaacaccgctactctgactatttcccgtgttgaagccggcgatgaagccgactactattgccaggtctgggacaccagctccgaccacgtagtctttggcgggggcaccaaactgaccgttttg

SEQ ID NO: 126 - DNA, 735 bp, synthetic construct
  misc_feature 1..735: note = Description of Artificial Sequence: Synthetic polynucleotide
  source 1..735: mol_type = other DNA
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtgggaattaacctccgaagcattcgatctttggggccagggcactatggtgaccgttagctctggcggtggtggtagcggaggcggaggatcaggtggaggcggcagttcttacgtgctgactcaaccaccatcagtgtctgtagcaccaggccagaccgcacgtattacctgtggcggtaacctgatcggctctaagctggttcactggtatcagcaaaaaccaggccaggcaccagtactggttgtgtacgatgatagcgatcgtccaagcgaaattccagagcgtttcagcggctctaattccggcaacaccgctactctgactatttcccgtgttgaagccggcgatgaagccgactactattgccaggtctgggactccagctccgacggtgtagtctttggcgggggcaccaaactgaccgttttg

SEQ ID NO: 127 - DNA, 735 bp, synthetic construct
  misc_feature 1..735: note = Description of Artificial Sequence: Synthetic polynucleotide
  source 1..735: mol_type = other DNA
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtggctgttagtagcggaagcattcgatctctggggccagggcactatggtgaccgttagctctggcggtggtggtagcggaggcggaggatcaggtggaggcggcagttcttacgtgctgactcaaccaccatcagtgtctgtagcaccaggccagaccgcacgtattacctgtggcggtaacatcatcggctctaagctggttcactggtatcagcaaaaaccaggccaggcaccagtactggttgtgtacgatgatagcgatcgtccaagcggtattccagagcgtttcagcggctctaattccggcaacaccgctactctgactatttcccgtgttgaagccggcgatgaagccgactactattgccaggtctgggactctggctccgaccacgtagtctttggcgggggcaccaaactgaccgttttg

SEQ ID NO: 128 - DNA, 735 bp, synthetic construct
  misc_feature 1..735: note = Description of Artificial Sequence: Synthetic polynucleotide
  source 1..735: mol_type = other DNA
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtgggaattagtagcggaagcattcgatctttggggccagggcactatggtgaccgttagctctggcggtggtggtagcggaggcggaggatcaggtggaggcggcagttcttacgtgctgactcaaccaccatcagtgtctgtagcaccaggccagaccgcacgtattacctgtggcggtaacatcatcggctctaagctggttcactggtatcagcaaaaaccaggccaggcaccagtactggttgtgtacgatgatagcgatcgtccaagcgatattccagagcgtttcagcggctctaattccggcaacaccgctactctgactatttcccgtgttgaagccggcgatgaagccgactactattgccaggtctgggactccggctccgacggtgtagtctttggcgggggcaccaaactgaccgttttg

SEQ ID NO: 129 - DNA, 735 bp, synthetic construct
  misc_feature 1..735: note = Description of Artificial Sequence: Synthetic polynucleotide
  source 1..735: mol_type = other DNA
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtgggaattagtagcggaagcattcgatctgtggggccagggcactatggtgaccgttagctctggcggtggtggtagcggaggcggaggatcaggtggaggcggcagttcttacgtgctgactcaaccaccatcagtgtctgtagcaccaggccagaccgcacgtattacctgtggcggtaacctgatcggctctaagctggttcactggtatcagcaaaaaccaggccaggcaccagtactggttgtgtacgatgatggcgatcgtccaagctggattccagagcgtttcagcggctctaattccggcaacaccgctactctgactatttcccgtgttgaagccggcgatgaagccgactactattgccaggtctgggactctggctccgacggtgtagtctttggcgggggcaccaaactgaccgttttg

SEQ ID NO: 130 - DNA, 735 bp, synthetic construct
  misc_feature 1..735: note = Description of Artificial Sequence: Synthetic polynucleotide
  source 1..735: mol_type = other DNA
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtgggtcttagtatccgaagcattcgatctttggggccagggcactatggtgaccgttagctctggcggtggtggtagcggaggcggaggatcaggtggaggcggcagttcttacgtgctgactcaaccaccatcagtgtctgtagcaccaggccagaccgcacgtattacctgtggcggtaacctgatcggctctaagctggttcactggtatcagcaaaaaccaggccaggcaccagtactggttgtgtacgatgatagcgatcgtccaagccgtattccagagcgtttcagcggctctaattccggcaacaccgctactctgactatttcccgtgttgaagccggcgatgaagccgactactattgccaggtctgggactccggctccgacggtgtagtctttggcgggggcaccaaactgaccgttttg

SEQ ID NO: 131 - DNA, 735 bp, synthetic construct
  misc_feature 1..735: note = Description of Artificial Sequence: Synthetic polynucleotide
  source 1..735: mol_type = other DNA
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtgggaattagtagcggaggcattcgatctgtggggccagggcactatggtgaccgttagctctggcggtggtggtagcggaggcggaggatcaggtggaggcggcagttcttacgtgctgactcaaccaccatcagtgtctgtagcaccaggccagaccgcacgtattacctgtggcggtaacatcatcggctctaagctggttcactggtatcagcaaaaaccaggccaggcaccagtactggttgtgtacgatgatagcgatcgtccaagcgcaattccagagcgtttcagcggctctaattccggcaacaccgctactctgactatttcccgtgttgaagccggcgatgaagccgactactattgccaggtctgggactccagctccgacggtgtagtctttggcgggggcaccaaactgaccgttttg

SEQ ID NO: 132 - DNA, 735 bp, synthetic construct
  misc_feature 1..735: note = Description of Artificial Sequence: Synthetic polynucleotide
  source 1..735: mol_type = other DNA
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctcttatgatggtagcaataaacactatgctgactcagtcaaaggccgtttcaccatcacccgtgataacagcaagaacactcttaacttacagatgaactctctgcgtgccgaagacaccgccgtttactactgtgcccgtagcccacagtgggaatgggtacacgaagcattcgatatgtggggccagggcactatggtgaccgttagctctggcggtggtggtagcggaggcggaggatcaggtggaggcggcagttcttacgtgctgactcaaccaccatcagtgtctgtagcaccaggccagaccgcacgtattacctgtggcggtaacaacatcggctctaagctggttcactggtatcagcaaaaaccaggccaggcaccagtactggttgtgtacgatgatagcgatcgtccaagcggtattccagagcgtttcagcggctctaattccggcaacaccgctactctgactatttcccgtgttgaagccggcgatgaagccgactactattgccaggtctgggactctagctccgacggtgtagtctttggcgggggcaccaaactgaccgttttg

SEQ ID NO: 133 - DNA, 735 bp, synthetic construct
  misc_feature 1..735: note = Description of Artificial Sequence: Synthetic polynucleotide
  source 1..735: mol_type = other DNA
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctcttatgatggtagcaataaacactatgctgactcagtcaaaggccgtttcaccatcacccgtgataacagcaagaacactcttaacttacagatgaactctctgcgtgccgaagacaccgccgtttactactgtgcccgttcgccacagtgggaatgggtacacgaagcattcgatctctggggccagggcactatggtgaccgttagctctggcggtggtggtagcggaggcggaggatcaggtggaggcggcagttcttacgtgctgactcaaccaccatcagtgtctgtagcaccaggccagaccgcacgtattacctgtggcggtaacatcctgggctctaagctggttcactggtatcagcaaaaaccaggccaggcaccagtactggttgtgtacgatgatagcgatcgtccaagcgaaattccagagcgtttcagcggctctaattccggcaacaccgctactctgactatttcccgtgttgaagccggcgatgaagccgactactattgccaggtctgggacaccagctccgacggtgtagtctttggcgggggcaccaaactgaccgttttg

SEQ ID NO: 134 - DNA, 735 bp, synthetic construct
  misc_feature 1..735: note = Description of Artificial Sequence: Synthetic polynucleotide
  source 1..735: mol_type = other DNA
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtagcccacagtgggaatgggtacacgaagcattcgatctatggggccagggcactatggtgaccgttagctctggcggtggtggtagcggaggcggaggatcaggtggaggcggcagttcttacgtgctgactcaaccaccatcagtgtctgtagcaccaggccagaccgcacgtattacctgtggcggtaacaacatcggctctaagctggttcactggtatcagcaaaaaccaggccaggcaccagtactggttgtgtacgatgatagcgatcgtccaagccggattccagagcgtttcagcggctctaattccggcaacaccgctactctgactatttcccgtgttgaagccggcgatgaagccgactactattgccaggtctgggacactagctccgacggtgtagtctttggcgggggcaccaaactgaccgttttg

SEQ ID NO: 135 - DNA, 735 bp, synthetic construct
  misc_feature 1..735: note = Description of Artificial Sequence: Synthetic polynucleotide
  source 1..735: mol_type = other DNA
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagtaatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgttcgccacagtgggaatgggtacacgaagcattcgatctctggggccagggcactatggtgaccgttagctctggcggtggtggtagcggaggcggaggatcaggtggaggcggcagttcttacgtgctgactcaaccaccatcagtgtctgtagcaccaggccagaccgcacgtattacctgtggcggtaacctgatcggctctaagctggttcactggtatcagcaaaaaccaggccaggcaccagtactggttgtgtacgatgatggcgatcgtccaagctggattccagagcgtttcagcggctctaattccggcaacaccgctactctgactatttcccgtgttgaagccggcgatgaagccgactactattgccaggtctgggactccagctccgacggtgtagtctttggcgggggcaccaaactgaccgttttg

SEQ ID NO: 136 - protein, 8 aa, synthetic construct
  REGION 1..8: note = Description of Artificial Sequence: Synthetic peptide
GFTFRTYG

SEQ ID NO: 137 - protein, 8 aa, synthetic construct
  REGION 1..8: note = Description of Artificial Sequence: Synthetic peptide
  VARIANT 2: note = MOD_RES - Trp or Ser
IXYDGSNK

SEQ ID NO: 138 - protein, 15 aa, synthetic construct
  REGION 1..15: note = Description of Artificial Sequence: Synthetic peptide
  VARIANT 3: note = MOD_RES - Ala or Ser
  VARIANT 7: note = MOD_RES - Glu, Gln, Met, Leu or Val
  VARIANT 8: note = MOD_RES - Leu, Trp or Tyr
  VARIANT 9: note = MOD_RES - Val or Thr
  VARIANT 10: note = MOD_RES - His, Ala or Ser
  VARIANT 15: note = MOD_RES - Ile, Leu or Met
ARXPQWXXXXEAFDX

SEQ ID NO: 139 - protein, 7 aa, synthetic construct
  REGION 1..7: note = Description of Artificial Sequence: Synthetic peptide
  VARIANT 1: note = MOD_RES - Asn, Leu or Ile
  VARIANT 2: note = MOD_RES - Leu or Ile
  VARIANT 6: note = MOD_RES - Ser or Leu
XXGSKXV

SEQ ID NO: 140 - skipped

SEQ ID NO: 141 - protein, 11 aa, synthetic construct
  REGION 1..11: note = Description of Artificial Sequence: Synthetic peptide
  VARIANT 5: note = MOD_RES - Ser or Thr
  VARIANT 6: note = MOD_RES - Ser or Gly
  VARIANT 9: note = MOD_RES - His or Gly
QVWDXXSDXVV

SEQ ID NO: 142 - protein, 8 aa, synthetic construct
  REGION 1..8: note = Description of Artificial Sequence: Synthetic peptide
  VARIANT 2: note = MOD_RES - Any amino acid
IXYDGSNK

SEQ ID NO: 143 - protein, 15 aa, synthetic construct
  REGION 1..15: note = Description of Artificial Sequence: Synthetic peptide
  VARIANT 3..12: note = MOD_RES - Any amino acid
  VARIANT 15: note = MOD_RES - Any amino acid
ARXXXXXXXXXXFDX

SEQ ID NO: 144 - protein, 7 aa, synthetic construct
  REGION 1..7: note = Description of Artificial Sequence: Synthetic peptide
  VARIANT 1..2: note = MOD_RES - Any amino acid
  VARIANT 6: note = MOD_RES - Any amino acid
XXGSKXV

SEQ ID NO: 145 - skipped

SEQ ID NO: 146 - protein, 11 aa, synthetic construct
  REGION 1..11: note = Description of Artificial Sequence: Synthetic peptide
  VARIANT 5..6: note = MOD_RES - Any amino acid
  VARIANT 9: note = MOD_RES - Any amino acid
QVWDXXSDXVV

SEQ ID NO: 147 - protein, 15 aa, synthetic construct
  REGION 1..15: note = Description of Artificial Sequence: Synthetic peptide
GGGGSGGGGSGGGGS

SEQ ID NO: 148 - DNA, 45 bp, synthetic construct
  misc_feature 1..45: note = Description of Artificial Sequence: Synthetic oligonucleotide
  source 1..45: mol_type = other DNA
ggcggtggtggtagcggaggcggaggatcaggtggaggcggcagt

SEQ ID NO: 149 - protein, 8 aa, synthetic construct
  REGION 1..8: note = Description of Artificial Sequence: Synthetic peptide
GFAFRTYG

SEQ ID NO: 150 - protein, 8 aa, synthetic construct
  REGION 1..8: note = Description of Artificial Sequence: Synthetic peptide
IWYDGSNT

SEQ ID NO: 151 - protein, 15 aa, synthetic construct
  REGION 1..15: note = Description of Artificial Sequence: Synthetic peptide
ARAPQWYLSAEAFDL

SEQ ID NO: 152 - protein, 6 aa, synthetic construct
  REGION 1..6: note = Description of Artificial Sequence: Synthetic peptide
LIGSKL

SEQ ID NO: 153 - skipped

SEQ ID NO: 154 - protein, 11 aa, synthetic construct
  REGION 1..11: note = Description of Artificial Sequence: Synthetic peptide
QVWDHSSDHVV

SEQ ID NO: 155 - protein, 122 aa, synthetic construct
  REGION 1..122: note = Description of Artificial Sequence: Synthetic polypeptide
QMQLVESGGGVVQPGRSLRLSCAASGFAFRTYGMHWVRQAPGKGLEWVAVIWYDGSNTHYADSVKGRFTITRDNSKNTLNLQMNSLRAEDTAVYYCARAPQWYLSAEAFDLWGQGTMVTVSS

SEQ ID NO: 156 - protein, 108 aa, synthetic construct
  REGION 1..108: note = Description of Artificial Sequence: Synthetic polypeptide
SYVLTQPPSVSVAPGQTARITCGGNLIGSKLVHWYQQKPGQAPVLVVYDDSDRPSRIPERFSGSNSGNTATLTISRVEAGDEADYYCQVWDHSSDHVVFGGGTKLTVL

SEQ ID NO: 157 - protein, 122 aa, synthetic construct
  REGION 1..122: note = Description of Artificial Sequence: Synthetic polypeptide
QVQLVQSGAEVKKPGASVKVSCKASGFAFRTYGLSWVRQAPGQGLEWMGWIWYDGSNTNYGQEFQGRVTMTTDTSTSTAYMELRSLRSDDTAVYYCARAPQWYLSAEAFDLWGRGTLVTVSS

SEQ ID NO: 158 - protein, 108 aa, synthetic construct
  REGION 1..108: note = Description of Artificial Sequence: Synthetic polypeptide
SYVLTQPPSVSVAPGKTARITCGGNLIGSKLVHWYQQKPGQAPVLVIYDDSDRPSRIPERFSGSNSGNTATLTISRVEAGDEADYYCQVWDHSSDHVVFGGGTKLTVL

SEQ ID NO: 159 - protein, 122 aa, synthetic construct
  REGION 1..122: note = Description of Artificial Sequence: Synthetic polypeptide
QMQLVESGGGVVQPGRSLRLSCAASGFTFRTYGMHWVRQAPGKGLEWVADIWYDGSNKHYADSVKGRFTITRDNSKNTLNLQMNSLRAEDTAVYYCARAPQWYLVAEPFDLWGQGTMVTVSS

SEQ ID NO: 160 - protein, 108 aa, synthetic construct; identical to SEQ ID NO: 3
  REGION 1..108: note = Description of Artificial Sequence: Synthetic polypeptide

SEQ ID NO: 161 - DNA, 366 bp, synthetic construct
  misc_feature 1..366: note = Description of Artificial Sequence: Synthetic polynucleotide
  source 1..366: mol_type = other DNA
caaatgcagctggtcgagtctggcggtggggtagtgcaaccaggccgttctctgcgtcttagctgcgccgcatctggttttgcgtttcgtacctacggtatgcactgggtgcgtcaggcaccaggcaaaggtctggaatgggtcgcagtaatctggtatgatggtagcaatacccactatgctgactcagtcaaaggccgtttcaccatcacccgtgataacagcaagaacactcttaacttacagatgaactctctgcgtgccgaagacaccgccgtttactactgtgcccgtgcaccacagtggtacttaagcgcggaagcattcgatctatggggccagggcactatggtgaccgttagctct

SEQ ID NO: 162 - DNA, 324 bp, synthetic construct
  misc_feature 1..324: note = Description of Artificial Sequence: Synthetic polynucleotide
  source 1..324: mol_type = other DNA
tcttacgtgctgactcaaccaccatcagtgtctgtagcaccaggccagaccgcacgtattacctgtggcggtaacctgatcggctctaagctggttcactggtatcagcaaaaaccaggccaggcaccagtactggttgtgtacgatgatagcgatcgtccaagccgtattccagagcgtttcagcggctctaattccggcaacaccgctactctgactatttcccgtgttgaagccggcgatgaagccgactactattgccaggtctgggaccacagctccgaccatgtagtctttggcgggggcaccaaactgaccgttttg

SEQ ID NO: 163 - DNA, 366 bp, synthetic construct
  misc_feature 1..366: note = Description of Artificial Sequence: Synthetic polynucleotide
  source 1..366: mol_type = other DNA
caaatgcagctggtcgagtctggcggtggggtagtgcaaccaggccgttctctgcgtcttagctgcgccgcatctggttttacctttcgtacctacggtatgcactgggtgcgtcaggcaccaggcaaaggtctggaatgggtcgcagacatctggtatgatggtagcaataaacactatgctgactcagtcaaaggccgtttcaccatcacccgtgataacagcaagaacactcttaacttacagatgaactctctgcgtgccgaagacaccgccgtttactactgtgcccgtgcaccacagtggtacttagtagcggaaccgttcgatctatggggccagggcactatggtgaccgttagctct

SEQ ID NO: 164 - DNA, 324 bp, synthetic construct; identical to SEQ ID NO: 58
  misc_feature 1..324: note = Description of Artificial Sequence: Synthetic polynucleotide
  source 1..324: mol_type = other DNA

SEQ ID NO: 165 - protein, 8 aa, synthetic construct
  REGION 1..8: note = Description of Artificial Sequence: Synthetic peptide
GFTFRTYG

SEQ ID NO: 166 - protein, 8 aa, synthetic construct
  REGION 1..8: note = Description of Artificial Sequence: Synthetic peptide
IWYDGSNK

SEQ ID NO: 167 - protein, 15 aa, synthetic construct
  REGION 1..15: note = Description of Artificial Sequence: Synthetic peptide
ARAPQWYLVAEPFDL

SEQ ID NO: 168 - protein, 11 aa, synthetic construct
  REGION 1..11: note = Description of Artificial Sequence: Synthetic peptide
QVWDSSSDGVV

SEQ ID NO: 169 - protein, 122 aa, synthetic construct
  REGION 1..122: note = Description of Artificial Sequence: Synthetic polypeptide
QMQLVESGGGVVQPGRSLRLSCAASGFAFRTYGMHWVRQAPGKGLEWVAVIWYDGSNTHYADSVKGRFTITRDNSKNTLNLQMNSLRAEDTAVYYCVRAPQWYLSAEAFDLWGQGTMVTVSS

SEQ ID NO: 170 - protein, 108 aa, synthetic construct
  REGION 1..108: note = Description of Artificial Sequence: Synthetic polypeptide
SYVLTQPPSVSVAPGQTARITCGGNLIGAKLVHWYQQKPGQAPVLVVYDDSDRPSRIPERFSGSNSGNTATLTISRVEAGDEADYYCQVWDHSSDHVVFGGGTKLTVL

SEQ ID NO: 171 - protein, 122 aa, synthetic construct
  REGION 1..122: note = Description of Artificial Sequence: Synthetic polypeptide
QMQLVESGGGVVQPGRSLRLSCAASGFAFRTYGMHWVRQAPGKGLEWVAVIWDDGSNTHYADSVKGRFTITRDNSKNTLNLQMNSLRAEDTAVYYCARAPQWYLSAEAFDLWGQGTMVTVSS

SEQ ID NO: 172 - protein, 108 aa, synthetic construct; identical to SEQ ID NO: 156
  REGION 1..108: note = Description of Artificial Sequence: Synthetic polypeptide

SEQ ID NO: 173 - protein, 122 aa, synthetic construct; identical to SEQ ID NO: 169
  REGION 1..122: note = Description of Artificial Sequence: Synthetic polypeptide

SEQ ID NO: 174 - protein, 108 aa, synthetic construct
  REGION 1..108: note = Description of Artificial Sequence: Synthetic polypeptide
SYVLTQPPSVSVAPGQTARITCGGNLIGSKLVHWYQQKPGQAPVLVVYDDSDRPSRIPERFSGSNSGNTATLTISRVEAGDEADYYCQVWDYSSNHVVFGGGTKLTVL

SEQ ID NO: 175 - protein, 122 aa, synthetic construct
  REGION 1..122: note = Description of Artificial Sequence: Synthetic polypeptide
QMQLVESGGGVVQPGRSLRLSCAASGFAFDTYGMHWVRQAPGKGLEWVAVIWYDGSNTVYADSVKGRFTITRDNSKNTLNLQMNSLRAEDTAVYYCARAPQWYLSAEAFDLWGQGTMVTVSS

SEQ ID NO: 176 - protein, 108 aa, synthetic construct
  REGION 1..108: note = Description of Artificial Sequence: Synthetic polypeptide
SYVLTQPPSVSVAPGQTARITCGGNLIGSKLVHWYQQKPGQAPVLVVYDDSDRPSRIPERFSGSNSGNTATLTISRVEAGDEADYYCQVWDHSSDHYVFGGGTKLTVL

SEQ ID NO: 177 - protein, 122 aa, synthetic construct; identical to SEQ ID NO: 169
  REGION 1..122: note = Description of Artificial Sequence: Synthetic polypeptide

SEQ ID NO: 178 - protein, 108 aa, synthetic construct
  REGION 1..108: note = Description of Artificial Sequence: Synthetic polypeptide
SYVLTQPPSVSVAPGETATITCGGNLIGSKLVHWYQQKPGQAPVLVVYDDSDRPSRIPERFSGSNSGNTATLTISRVEAGDEADYYCQVWDHSSDHVVFGGGTKLTVL

SEQ ID NO: 179 - protein, 122 aa, synthetic construct
  REGION 1..122: note = Description of Artificial Sequence: Synthetic polypeptide
QMQLVESGGGVVQPGRSLRLSCAASGFAFRTYGMHWVRQAPGKGLEWVAVIWYDGSATHYADSVKGRFTITRDNSKNTLNLQMNSLRAEDTAVYYCVRAPQWYLSAEAFDLWGQGTMVTVSS

SEQ ID NO: 180 - protein, 108 aa, synthetic construct
  REGION 1..108: note = Description of Artificial Sequence: Synthetic polypeptide
SYVLTQPPSVSVAPGQTATITCGGNLIGSKLVHWYQQKPGQAPVLVVYDDSDRPSRIPERFSGSNIGNTATLTISRVEAGDEADYYCQVWDHSSDHVVFGGGTKLTVL

SEQ ID NO: 181 - protein, 122 aa, synthetic construct
  REGION 1..122: note = Description of Artificial Sequence: Synthetic polypeptide
QMQLVESGGGVVQPGRSLRLSCAASGFDFRTYGMHWVRQAPGKGLEWVAVIWYDGSITHYADSVKGRFTITRDNSKNTLNLQMNSLRAEDTAVYYCVRAPQWYLTAEAFDLWGQGTMVTVSS

SEQ ID NO: 182 - protein, 108 aa, synthetic construct
  REGION 1..108: note = Description of Artificial Sequence: Synthetic polypeptide
SYVLTQPPSVSVAPGQTARITCGGNLIGTKLVHWYQQKPGQAPVLVVYDDSDRPSRIPERFSGSNSGNTATLTISRVEAGDEADYYCQVWDHNEDEVVFGGGTKLTVL

SEQ ID NO: 183 - DNA, 366 bp, synthetic construct
  misc_feature 1..366: note = Description of Artificial Sequence: Synthetic polynucleotide
  source 1..366: mol_type = other DNA
cagatgcagttggtggagtccggaggtggagtggtgcaaccagggcgttccttgcgtttgtcttgtgctgcttccggattcgcctttcgtacatatggcatgcattgggtgcgtcaagcacctggtaagggcctggagtgggttgccgttatttggtacgacggctccaacacccactacgcagatagcgtgaaaggacgtttcactattacccgtgataactccaagaatacccttaacctgcagatgaatagcttgcgtgctgaggacacagcagtatattactgcgtccgtgcaccacaatggtacctgagcgccgaggcctttgatctgtgggggcagggcacaatggtgaccgtttcctca

SEQ ID NO: 184 - DNA, 324 bp, synthetic construct
  misc_feature 1..324: note = Description of Artificial Sequence: Synthetic polynucleotide
  source 1..324: mol_type = other DNA
agttacgtgctgacacaacctccaagtgttagtgtcgcaccaggacaaacagcacgtattacatgtggaggaaatcttatcggtgccaagctggtgcactggtaccagcagaaacctggtcaggccccagtactggttgtgtatgatgacagcgaccgtccaagccgtatcccagaacgtttttctgggagcaactcaggtaatacagccactctgaccatttcacgtgttgaggcaggagatgaggccgattattattgccaagtatgggaccacagctctgaccatgttgtttttggcggagggactaagctgaccgtgctt

SEQ ID NO: 185 - DNA, 366 bp, synthetic construct
  misc_feature 1..366: note = Description of Artificial Sequence: Synthetic polynucleotide
  source 1..366: mol_type = other DNA
cagatgcaactggtggagtcaggaggcggcgtggtgcagccaggacgttctctgcgtctgtcttgcgcagcttccgggttcgcctttcgtacctatgggatgcattgggtgcgtcaggctccaggtaagggactggagtgggtcgctgttatttgggacgacggaagtaacactcattacgccgacagcgtgaagggccgtttcacaattacccgtgacaattccaagaataccttgaacctgcagatgaactctcttcgtgctgaagataccgccgtgtactattgcgcccgtgctccacagtggtatctgtcagcagaggccttcgacctgtggggacagggaacaatggtgaccgtatcttca

SEQ ID NO: 186 - DNA, 324 bp, synthetic construct
  misc_feature 1..324: note = Description of Artificial Sequence: Synthetic polynucleotide
  source 1..324: mol_type = other DNA
tcctatgtgctgacacagccacctagcgtgagcgtcgccccaggtcagaccgctcgtatcacttgtggcgggaaccttatcggcagcaagctggtgcactggtaccagcagaagcctggccaagcacctgtgctggtcgtttatgacgactctgaccgtccatcccgtatcccagaacgtttctctggctctaactctgggaataccgctaccctgacaatctcacgtgttgaagctggcgacgaggcagattattattgccaagtctgggatcactccagcgatcacgtcgtgttcggaggcggaacaaaattgactgtcctg

SEQ ID NO: 187 - DNA, 366 bp, synthetic construct
  misc_feature 1..366: note = Description of Artificial Sequence: Synthetic polynucleotide
  source 1..366: mol_type = other DNA
caaatgcagctggtggaatccgggggtggggtcgtccagcctggccgtagtctgcgtctttcctgtgccgcatcaggctttgctttccgtacctacgggatgcactgggtgcgtcaggccccaggaaagggacttgaatgggtggctgtcatctggtacgatggttccaacacacactatgccgattcagtgaaagggcgtttcaccattactcgtgacaatagtaagaatactctgaatctgcaaatgaattcactgcgtgctgaggacaccgctgtttattactgtgtgcgtgctcctcagtggtacctgagtgccgaagctttcgatttgtggggacagggcacaatggtgacagtcagttct

SEQ ID NO: 188 - DNA, 324 bp, synthetic construct
  misc_feature 1..324: note = Description of Artificial Sequence: Synthetic polynucleotide
  source 1..324: mol_type = other DNA
tcttacgtgttgacacaaccaccaagtgttagtgtcgcacctggccaaaccgctcgtatcacctgtggtgggaatcttattggctctaagctggtgcactggtatcagcagaaaccaggccaggctccagtactggtggtgtacgacgactctgaccgtccaagccgtatcccagagcgtttcagtggctctaactccgggaacacagcaactcttacaatttcacgtgtggaggccggtgatgaagccgactactattgccaggtttgggactacagtagtaatcacgtggttttcggtggtggtaccaagctgactgtgttg

SEQ ID NO: 189 - DNA, 366 bp, synthetic construct
  misc_feature 1..366: note = Description of Artificial Sequence: Synthetic polynucleotide
  source 1..366: mol_type = other DNA
cagatgcagctggttgaatctggcggcggtgtggtccagcctggtcgtagcctgcgtctgtcctgtgctgcaagcggatttgcctttgacacctatgggatgcactgggtacgtcaggccccaggaaagggcctggaatgggtggcagttatctggtatgatggttctaataccgtgtatgccgactccgttaaaggccgtttcactatcacccgtgataatagtaaaaacacactgaacctgcagatgaatagcttgcgtgctgaggacaccgcagtgtactactgtgcccgtgctcctcagtggtatctgtcagcagaggccttcgatctgtggggccaagggacaatggtgaccgtgtcttcc

SEQ ID NO: 190 - DNA, 324 bp, synthetic construct
  misc_feature 1..324: note = Description of Artificial Sequence: Synthetic polynucleotide
  source 1..324: mol_type = other DNA
agctatgttctgactcaaccacctagtgtgagtgtggcccctggtcagactgcacgtattacctgtggcggaaaccttatcggcagtaagctggttcattggtatcagcagaagccaggacaggcaccagtgctggtcgtttacgacgatagtgaccgtccatcacgtatcccagagcgttttagcgggtccaattccggaaatacagcaaccttgaccattagccgtgtggaagccggcgatgaagctgattattactgccaggtatgggaccattcctccgaccactacgtttttggtggcggaactaagctgacagtcttg

SEQ ID NO: 191 - DNA, 366 bp, synthetic construct
  misc_feature 1..366: note = Description of Artificial Sequence: Synthetic polynucleotide
  source 1..366: mol_type = other DNA
cagatgcagcttgttgagagcggcggaggcgtggtgcaaccaggccgttcattgcgtctgtcctgcgccgccagcggctttgcttttcgtacatacggcatgcactgggtgcgtcaggcccctggcaaggggctggaatgggtcgccgtgatttggtatgacggtagtaacacccattatgctgattccgtcaagggacgtttcactatcacccgtgacaatagcaaaaatacactgaatctgcaaatgaattcattgcgtgccgaagacaccgccgtatattactgtgtccgtgccccacagtggtacctgagcgctgaggccttcgatctgtggggtcaggggactatggtgaccgtatcatcc

SEQ ID NO: 192 - DNA, 324 bp, synthetic construct
  misc_feature 1..324: note = Description of Artificial Sequence: Synthetic polynucleotide
  source 1..324: mol_type = other DNA
tcttacgtgcttactcagcctcctagcgtctcagtggccccaggcgagacagcaaccattacatgcgggggtaatttgatcggtagcaagctggtgcattggtatcagcagaagcctggccaggccccagtgctggttgtatatgacgatagtgatcgtccaagtcgtatccctgagcgttttagcggatctaactccggcaacacagccacattgacaatcagccgtgtggaggcaggcgatgaggccgactactactgccaagtttgggaccactcctctgaccacgtggtatttggcggaggaacaaagcttacagttttg

SEQ ID NO: 193 - DNA, 366 bp, synthetic construct
  misc_feature 1..366: note = Description of Artificial Sequence: Synthetic polynucleotide
  source 1..366: mol_type = other DNA
cagatgcagctggtggagagtggaggtggtgtggtgcaacctgggcgtagcctgcgtttgagctgcgctgcctctggatttgccttccgtacctatggcatgcactgggtgcgtcaggctccaggaaaggggttggaatgggtggctgtgatttggtacgacgggagcgccacacattacgcagacagcgttaagggccgtttcacaattacccgtgacaatagcaaaaatacattgaacctgcagatgaattccctgcgtgcagaggatactgcagtgtactattgcgtccgtgccccacagtggtatctgtcagccgaagccttcgatctgtgggggcagggtactatggtcaccgtaagttcc

SEQ ID NO: 194 - DNA, 324 bp, synthetic construct
  misc_feature 1..324: note = Description of Artificial Sequence: Synthetic polynucleotide
  source 1..324: mol_type = other DNA
tcttatgttttgacccaacctccatccgttagcgtggctccaggtcaaacagctaccatcacatgtggcggtaaccttattggctcaaagctggttcattggtatcaacagaaaccaggccaagccccagtgctggtggtgtatgacgacagtgaccgtccttctcgtattcctgagcgtttttccggctctaatattggcaacactgccaccctgaccatttctcgtgtggaagcaggagatgaggcagactattattgtcaggtttgggatcactccagcgatcatgtggtattcggaggtgggacaaaacttactgttctt

SEQ ID NO: 195 - DNA, 366 bp, synthetic construct
  misc_feature 1..366: note = Description of Artificial Sequence: Synthetic polynucleotide
  source 1..366: mol_type = other DNA
cagatgcagctggtggaaagtggtgggggagtcgtgcaaccaggacgttccttgcgtctgtcatgcgctgcttcaggtttcgactttcgtacctacggcatgcattgggtgcgtcaggctccaggtaaaggacttgagtgggtcgcagtgatctggtacgacggatcaattactcactacgccgatagcgtgaaaggccgtttcaccatcacccgtgacaactccaagaacaccctgaacttgcagatgaacagtctgcgtgcagaagacactgcagtatattattgtgtccgtgccccacagtggtacttgaccgccgaggcttttgatctgtggggacagggcacaatggtgaccgtatctagc

SEQ ID NO: 196 - DNA, 324 bp, synthetic construct
  misc_feature 1..324: note = Description of Artificial Sequence: Synthetic polynucleotide
  source 1..324: mol_type = other DNA
agctacgtgcttacccagccaccatcagtcagtgtggctccaggccaaactgcccgtatcacctgcggcggcaatttgattggcaccaagcttgtgcactggtaccaacagaagccagggcaggcccctgtgctggttgtctacgacgatagtgatcgtccttcccgtattcctgaacgtttctctggaagcaattccggaaacacagccacacttaccatttctcgtgttgaggctggggatgaagccgactactattgccaggtttgggaccacaatgaagacgaagttgtttttggaggaggaactaagctgacagttctg

SEQ ID NO: 197 - protein, 8 aa, synthetic construct
  REGION 1..8: note = Description of Artificial Sequence: Synthetic peptide
GFAFDTYG

SEQ ID NO: 198 - protein, 8 aa, synthetic construct
  REGION 1..8: note = Description of Artificial Sequence: Synthetic peptide
GFDFRTYG

SEQ ID NO: 199 - protein, 8 aa, synthetic construct
  REGION 1..8: note = Description of Artificial Sequence: Synthetic peptide
IWYDGSAT

SEQ ID NO: 200 - protein, 8 aa, synthetic construct
  REGION 1..8: note = Description of Artificial Sequence: Synthetic peptide
IWYDGSIT

SEQ ID NO: 201 - protein, 15 aa, synthetic construct
  REGION 1..15: note = Description of Artificial Sequence: Synthetic peptide
VRAPQWYLSAEAFDL

SEQ ID NO: 202 - protein, 15 aa, synthetic construct
  REGION 1..15: note = Description of Artificial Sequence: Synthetic peptide
VRAPQWYLTAEAFDL

SEQ ID NO: 203 - protein, 6 aa, synthetic construct
  REGION 1..6: note = Description of Artificial Sequence: Synthetic peptide
LIGAKL

SEQ ID NO: 204 - protein, 6 aa, synthetic construct
  REGION 1..6: note = Description of Artificial Sequence: Synthetic peptide
LIGTKL

SEQ ID NO: 205 - protein, 11 aa, synthetic construct
  REGION 1..11: note = Description of Artificial Sequence: Synthetic peptide
QVWDYSSNHVV

SEQ ID NO: 206 - protein, 11 aa, synthetic construct
  REGION 1..11: note = Description of Artificial Sequence: Synthetic peptide
QVWDHSSDHYV

SEQ ID NO: 207 - protein, 11 aa, synthetic construct
  REGION 1..11: note = Description of Artificial Sequence: Synthetic peptide
QVWDHNEDEVV

SEQ ID NO: 208 - protein, 8 aa, synthetic construct
  REGION 1..8: note = Description of Artificial Sequence: Synthetic peptide
IWDDGSNT

SEQ ID NO: 209 - protein, 122 aa, synthetic construct
  REGION 1..122: note = Description of Artificial Sequence: Synthetic polypeptide
QMQLVESGGGVVQPGRSLRLSCAASGFAFRTYGMHWVRQAPGKGLEWVAVIWDDGSNTHYADSVKGRFTITRDNSKNTLNLQMNSLRAEDTAVYYCVRAPQWYLTAEAFDLWGQGTMVTVSS

SEQ ID NO: 210 - protein, 108 aa, synthetic construct
  REGION 1..108: note = Description of Artificial Sequence: Synthetic polypeptide
SYVLTQPPSVSVAPGQTATITCGGNMIGAYLVHWYQQKPGQAPLLVVYDDVDRPNRIPERFSGSNSGNTATLTISRVEAGDEADYYCQVWDHNTDKMVFGGGTKLTVL

SEQ ID NO: 211 - protein, 122 aa, synthetic construct; identical to SEQ ID NO: 179
  REGION 1..122: note = Description of Artificial Sequence: Synthetic polypeptide

SEQ ID NO: 212 - protein, 108 aa, synthetic construct
  REGION 1..108: note = Description of Artificial Sequence: Synthetic polypeptide
SYVLTQPPSVSVAPGETATITCGGNLIGAYLVHWYQQKPGQAPVLVIYDDVDRPARIPERFSGSNSGNTATLTISRVEAGDEADYYCQVWDHDTNTMVFGGGTKLTVL

SEQ ID NO: 213 - protein, 122 aa, synthetic construct
  REGION 1..122: note = Description of Artificial Sequence: Synthetic polypeptide
QMQLVESGGGVVQPGRSLRLSCAASGFAFRTYGMHWVRQAPGKGLEWVAVIWDDGSATHYADSVKGRFTITRDNSKNTLNLQMNSLRAEDTAVYYCVRAPQWYLTAEAFDLWGQGTMVTVSS

SEQ ID NO: 214 - protein, 108 aa, synthetic construct
  REGION 1..108: note = Description of Artificial Sequence: Synthetic polypeptide
SYVLTQPPSVSVAPGETASITCGGNMIGGYLVHWYQQKPGQAPLLVIYDDVDRPARIPERFSGSNSGNTATLTISRVEAGDEADYYCQVWDHDTNHMVFGGGTKLTVL

SEQ ID NO: 215 - protein, 122 aa, synthetic construct
  REGION 1..122: note = Description of Artificial Sequence: Synthetic polypeptide
QMQLVESGGGVVQPGRSLTLSCAASGFAFRTYGMHWVRQAPGKGLEWVGVIWYDGSATHYADSVKGRFTITRDNSKNTLNLQMNSLRAEDTAVYYCVRAPQWYLTAEAFDLWGQGTMVTVSS

SEQ ID NO: 216 - protein, 108 aa, synthetic construct
  REGION 1..108: note = Description of Artificial Sequence: Synthetic polypeptide
SYVLTQPPSVSVAPGETATITCGGALIGSRLVHWYQQKPGQAPVLVIYDDSDRPSRIPERFSGSNIGNTATLTISDVEAGDEADYYCQVWDHSSNTYVFGGGTKLTVL

SEQ ID NO: 217 - protein, 122 aa, synthetic construct
  REGION 1..122: note = Description of Artificial Sequence: Synthetic polypeptide
QMQLVESGGGVVQPGRSLRLSCAASGFEFRTYGMHWVRQAPGKGLEWLGVIWYDGSNTHYADSVKGRFTITRDNSKNTLNLQMNSLRVEDTAVYYCVRAPQWYLTAEAFDLWGQGTMVTVSS

SEQ ID NO: 218 - protein, 108 aa, synthetic construct
  REGION 1..108: note = Description of Artificial Sequence: Synthetic polypeptide
SYILTQPPSVSVAPGETATITCGGNLIGARLVHWYQQKPGQAPVLVIYDDSDRPSRIPERFSGSNIGNTATLTIERVEAGDEADYYCQVWDYYSDHMVFGGGTKLTVL

SEQ ID NO: 219 - protein, 122 aa, synthetic construct
  REGION 1..122: note = Description of Artificial Sequence: Synthetic polypeptide
QMQLVESGGGVVQPGRSLRLSCAASGFAFRTYGMHWVRQAPGKGLEWVAVIWYDGSNTHYADSVKGRFTITRDNSKNTLNLQMNSLRAEDTAVYYCVRAPQWYLTAEAFDLWGQGTMVTVSS

SEQ ID NO: 220 - protein, 108 aa, synthetic construct
  REGION 1..108: note = Description of Artificial Sequence: Synthetic polypeptide
SYVLTQPPSVSVAPGETATITCGGNMIGGYLVHWYQQKPGQAPLLVIYDDVDRPDRIPERFSGSNSGNTATLTISRVEAGDEADYYCQVWDHDSNTMVFGGGTKLTVL

SEQ ID NO: 221 - protein, 122 aa, synthetic construct; identical to SEQ ID NO: 179
  REGION 1..122: note = Description of Artificial Sequence: Synthetic polypeptide

SEQ ID NO: 222 - protein, 108 aa, synthetic construct
  REGION 1..108: note = Description of Artificial Sequence: Synthetic polypeptide
SYVLTQPPSVSVAPGETATITCGGNLIGAYLVHWYQQKPGQAPVLVIYDDSDRPDRIPERFSGSNSGNTATLTISRVEAGDEADYYCQVWDHNTNHMVFGGGTKLTVL

SEQ ID NO: 223 - protein, 122 aa, synthetic construct; identical to SEQ ID NO: 209
  REGION 1..122: note = Description of Artificial Sequence: Synthetic polypeptide

SEQ ID NO: 224 - protein, 108 aa, synthetic construct
  REGION 1..108: note = Description of Artificial Sequence: Synthetic polypeptide
SYVLTQPPSVSVAPGQTARITCGGNMIGAYLVHWYQQKPGQAPLLVIYDDVDRPDRIPERFSGSNSGNTATLTISRVEAGDEADYYCQVWDHNTQQMVFGGGTKLTVL

SEQ ID NO: 225 - protein, 122 aa, synthetic construct; identical to SEQ ID NO: 209
  REGION 1..122: note = Description of Artificial Sequence: Synthetic polypeptide

SEQ ID NO: 226 - protein, 108 aa, synthetic construct
  REGION 1..108: note = Description of Artificial Sequence: Synthetic polypeptide
SYVLTQPPSVSVAPGETATITCGGNMIGGYLVHWYQQKPGQAPVLVIYDDVDRPDRIPERFSGSNSGNTATLTISRVEAGDEADYYCQVWDHDTNQVVFGGGTKLTVL

SEQ ID NO: 227 - protein, 122 aa, synthetic construct
  REGION 1..122: note = Description of Artificial Sequence: Synthetic polypeptide
QMQLVESGGGVVQPGRSLTLSCAASGFEFRTYGMHWVRQAPGKGLEWVGVIWDDGSNTHYADSVKGRFTITRDNSKNTLNLQMNSLRAEDTAVYYCARAPQWYLTAEAFDLWGQGTMVTVSS

SEQ ID NO: 228 - protein, 108 aa, synthetic construct
  REGION 1..108: note = Description of Artificial Sequence: Synthetic polypeptide
SYILTQPPSVSVAPGQTATITCGGNLIGAKLVHWYQQKPGQAPVLVIYDDSDRPSRIPERFSGSNIGNTATLTISDVEEGDEADYYCQVWDYSSDTMVFGGGTKLTVL

SEQ ID NO: 229 - protein, 122 aa, synthetic construct; identical to SEQ ID NO: 209
  REGION 1..122: note = Description of Artificial Sequence: Synthetic polypeptide

SEQ ID NO: 230 - protein, 108 aa, synthetic construct
  REGION 1..108: note = Description of Artificial Sequence: Synthetic polypeptide
SYVLTQPPSVSVAPGETATITCGGNLIGAYLVHWYQQKPGQAPVLVVYDDVDRPDRIPERFSGSNSGNTATLTISRVEAGDEADYYCQVWDHSTNTMVFGGGTKLTVL

SEQ ID NO: 231 - protein, 122 aa, synthetic construct
  REGION 1..122: note = Description of Artificial Sequence: Synthetic polypeptide
QMQLVESGGGVVQPGRSLTLSCAASGFEFRTYGMHWVRQAPGKGLEWVGVIWYDGSNTHYADSVKGRFTITRDNSKNTLNLQMNSLRVEDTAVYYCVRAPQWYLTAEAFDLWGQGTMVTVSS

SEQ ID NO: 232 - protein, 108 aa, synthetic construct
  REGION 1..108: note = Description of Artificial Sequence: Synthetic polypeptide
SYVLTQPPSVSVAPGETATITCGGNLIGARLVHWYQQKPGQAPVLVIYDDSDRPSRIPERFSGSNIGNTATLTIERVEAGDEADYYCQVWDYSSNSYVFGGGTKLTVL

SEQ ID NO: 233 - protein, 122 aa, synthetic construct
  REGION 1..122: note = Description of Artificial Sequence: Synthetic polypeptide
QMQLVESGGGVVQPGRSLRLSCAASGFEFRTYGMHWVRQAPGKGLEWVGVIWYDGSNTHYADSVKGRFTITRDNSKNTLNLQMNSLRAEDTAVYYCVRAPQWYLTAEAFDLWGQGTMVTVSS

SEQ ID NO: 234 - protein, 108 aa, synthetic construct
  REGION 1..108: note = Description of Artificial Sequence: Synthetic polypeptide
SYVLTQPPSVSVAPGQTARITCGGNLIGARLVHWYQQKPGQAPVLVVYDDSDRPSRIPERFSGSNIGNTATLTISDVEEGDEADYYCQVWDYSSNTYVFGGGTKLTVL

SEQ ID NO: 235 - protein, 122 aa, synthetic construct; identical to SEQ ID NO: 209
  REGION 1..122: note = Description of Artificial Sequence: Synthetic polypeptide

SEQ ID NO: 236 - protein, 108 aa, synthetic construct
  REGION 1..108: note = Description of Artificial Sequence: Synthetic polypeptide
SYVLTQPPSVSVAPGQTATITCGGNMIGAYLVHWYQQKPGQAPVLVIYDDVDRPDRIPERFSGSNSGNTATLTISRVEAGDEADYYCQVWDHNSNQMVFGGGTKLTVL

SEQ ID NO: 237 - protein, 122 aa, synthetic construct; identical to SEQ ID NO: 231
  REGION 1..122: note = Description of Artificial Sequence: Synthetic polypeptide

SEQ ID NO: 238 - protein, 108 aa, synthetic construct
  REGION 1..108: note = Description of Artificial Sequence: Synthetic polypeptide
SYVLTQPPSVSVAPGETATITCGGNLIGARLVHWYQQKPGQAPVLVIYDDSDRPSRIPERFSGSNIGNTATLTISDVEAGDEADYYCQVWDYSSNTYVFGGGTKLTVL

SEQ ID NO: 239 - protein, 122 aa, synthetic construct
  REGION 1..122: note = Description of Artificial Sequence: Synthetic polypeptide
QMQLVESGGGVVQPGRSLRLSCAASGFEFRTYGMHWVRQAPGKGLEWVGVIWYDGSATHYADSVKGRFTITRDNSKNTLNLQMNSLRVEDTAVYYCVRAPQWYLTAEAFDLWGQGTMVTVSS

SEQ ID NO: 240 - protein, 108 aa, synthetic construct
  REGION 1..108: note = Description of Artificial Sequence: Synthetic polypeptide
SYILTQPPSVSVAPGETATITCGGNLIGARLVHWYQQKPGQAPVLVIYDDSDRPSRIPERFSGSNIGNTATLTISRVEEGDEADYYCQVWDHSSNHYVFGGGTKLTVL

SEQ ID NO: 241 - protein, 122 aa, synthetic construct
  REGION 1..122: note = Description of Artificial Sequence: Synthetic polypeptide
QMQLVESGGGVVQPGRSLTLSCAASGFEFRTYGMHWVRQAPGKGLEWLGVIWDDGSNTHYADSVKGRFTITRDNSKNTLNLQMNSLRVEDTAVYYCARAPQWYLTAEAFDLWGQGTMVTVSS

SEQ ID NO: 242 - protein, 108 aa, synthetic construct
  REGION 1..108: note = Description of Artificial Sequence: Synthetic polypeptide
SYILTQPPSVSVAPGETARITCGGNLIGARLVHWYQQKPGQAPVLVVYDDSDRPSRIPERFSGSNIGNTATLTIEDVEEGDEADYYCQVWDYYSDHMVFGGGTKLTVL

SEQ ID NO: 243 - protein, 122 aa, synthetic construct
  REGION 1..122: note = Description of Artificial Sequence: Synthetic polypeptide
QMQLVESGGGVVQPGRSLTLSCAASGFEFRTYGMHWVRQAPGKGLEWLGVIWYDGSNTHYADSVKGRFTITRDNSKNTLNLQMNSLRVEDTAVYYCVRAPQWYLTAEAFDLWGQGTMVTVSS

SEQ ID NO: 244 - protein, 108 aa, synthetic construct
  REGION 1..108: note = Description of Artificial Sequence: Synthetic polypeptide
SYILTQPPSVSVAPGETATITCGGNLIGSRLVHWYQQKPGQAPVLVIYDDSDRPSRIPERFSGSNIGNTATLTISDVEAGDEADYYCQVWDHYSNHYVFGGGTKLTVL

SEQ ID NO: 245 - protein, 122 aa, synthetic construct
  REGION 1..122: note = Description of Artificial Sequence: Synthetic polypeptide
QMQLVESGGGVVQPGRSLTLSCAASGFEFRTYGMHWVRQAPGKGLEWLAVIWDDGSNTHYADSVKGRFTITRDNSKNTLNLQMNSLRVEDTAVYYCVRAPQWYLTAEAFDLWGQGTMVTVSS

SEQ ID NO: 246 - protein, 108 aa, synthetic construct
  REGION 1..108: note = Description of Artificial Sequence: Synthetic polypeptide
SYVLTQPPSVSVAPGETATITCGGNLIGARLVHWYQQKPGQAPVLVIYDDSDRPSRIPERFSGSNIGNTATLTISDVEEGDEADYYCQVWDYYSDHMVFGGGTKLTVL

SEQ ID NO: 247 - protein, 122 aa, synthetic construct
  REGION 1..122: note = Description of Artificial Sequence: Synthetic polypeptide
QMQLVESGGGVVQPGRSLTLSCAASGFEFRTYGMHWVRQAPGKGLEWVGVIWYDGSNTHYADSVKGRFTITRDNSKNTLNLQMNSLRAEDTAVYYCVRAPQWYLTAEAFDLWGQGTMVTVSS

SEQ ID NO: 248 - protein, 108 aa, synthetic construct
  REGION 1..108: note = Description of Artificial Sequence: Synthetic polypeptide
SYVLTQPPSVSVAPGQTARITCGGNLIGARLVHWYQQKPGQAPVLVIYDDSDRPSRIPERFSGSNIGNTATLTIERVEEGDEADYYCQVWDYSADSYVFGGGTKLTVL

SEQ ID NO: 249 - protein, 122 aa, synthetic construct; identical to SEQ ID NO: 209
  REGION 1..122: note = Description of Artificial Sequence: Synthetic polypeptide

SEQ ID NO: 250 - protein, 108 aa, synthetic construct
  REGION 1..108: note = Description of Artificial Sequence: Synthetic polypeptide
SYVLTQPPSVSVAPGETASITCGGNLIGAYLVHWYQQKPGQAPLLVIYDDVDRPARIPERFSGSNSGNTATLTISRVEAGDEADYYCQVWDHNSNHMVFGGGTKLTVL

SEQ ID NO: 251 - protein, 122 aa, synthetic construct
  REGION 1..122: note = Description of Artificial Sequence: Synthetic polypeptide
QMQLVESGGGVVQPGRSLTLSCAASGFEFRTYGMHWVRQAPGKGLEWVGVIWYDGSATHYADSVKGRFTITRDNSKNTLNLQMNSLRAEDTAVYYCVRAPQWYLTAEAFDLWGQGTMVTVSS

SEQ ID NO: 252 - protein, 108 aa, synthetic construct
  REGION 1..108: note = Description of Artificial Sequence: Synthetic polypeptide
SYVLTQPPSVSVAPGQTATITCGGALIGSRLVHWYQQKPGQAPVLVVYDDSDRPSRIPERFSGSNIGNTATLTISRVEEGDEADYYCQVWDYYSDSYVFGGGTKLTVL

SEQ ID NO: 253 - protein, 122 aa, synthetic construct
  REGION 1..122: note = Description of Artificial Sequence: Synthetic polypeptide
QMQLVESGGGVVQPGRSLRLSCAASGFEFRTYGMHWVRQAPGKGLEWVAVIWDDGSNTHYADSVKGRFTITRDNSKNTLNLQMNSLRVEDTAVYYCVRAPQWYLTAEAFDLWGQGTMVTVSS

SEQ ID NO: 254 - protein, 108 aa, synthetic construct
  REGION 1..108: note = Description of Artificial Sequence: Synthetic polypeptide
SYILTQPPSVSVAPGETARITCGGNLIGARLVHWYQQKPGQAPVLVIYDDSDRPSRIPERFSGSNIGNTATLTISRVEEGDEADYYCQVWDYSSDSMVFGGGTKLTVL

SEQ ID NO: 255 - protein, 122 aa, synthetic construct
  REGION 1..122: note = Description of Artificial Sequence: Synthetic polypeptide
QMQLVESGGGVVQPGRSLRLSCAASGFEFRTYGMHWVRQAPGKGLEWLGVIWYDGSATHYADSVKGRFTITRDNSKNTLNLQMNSLRAEDTAVYYCVRAPQWYLTAEAFDLWGQGTMVTVSS

SEQ ID NO: 256 - protein, 108 aa, synthetic construct
  REGION 1..108: note = Description of Artificial Sequence: Synthetic polypeptide
SYILTQPPSVSVAPGETATITCGGNLIGARLVHWYQQKPGQAPVLVIYDDSDRPSRIPERFSGSNIGNTATLTIERVEEGDEADYYCQVWDYYSDHYVFGGGTKLTVL

SEQ ID NO: 257 - protein, 122 aa, synthetic construct
  REGION 1..122: note = Description of Artificial Sequence: Synthetic polypeptide
QMQLVESGGGVVQPGRSLTLSCAASGFEFRTYGMHWVRQAPGKGLEWVAVIWDDGSNTVYADSVKGRFTITRDNSKNTLNLQMNSLRVEDTAVYYCVRAPQWYLTAEAFDLWGQGTMVTVSS

SEQ ID NO: 258 - protein, 108 aa, synthetic construct
  REGION 1..108: note = Description of Artificial Sequence: Synthetic polypeptide
SYVLTQPPSVSVAPGQTATITCGGNLIGARLVHWYQQKPGQAPVLVIYDDSDRPSRIPERFSGSNIGNTATLTISRVEAGDEADYYCQVWDYSSDSMVFGGGTKLTVL

SEQ ID NO: 259 - protein, 122 aa, synthetic construct; identical to SEQ ID NO: 233
  REGION 1..122: note = Description of Artificial Sequence: Synthetic polypeptide

SEQ ID NO: 260 - protein, 108 aa, synthetic construct
  REGION 1..108: note = Description of Artificial Sequence: Synthetic polypeptide
SYILTQPPSVSVAPGETARITCGGNLIGARLVHWYQQKPGQAPVLVIYDDSDRPSRIPERFSGSNIGNTATLTIEDVEEGDEADYYCQVWDYSSDSYVFGGGTKLTVL

SEQ ID NO: 261 - protein, 122 aa, synthetic construct
  REGION 1..122: note = Description of Artificial Sequence: Synthetic polypeptide
QMQLVESGGGVVQPGRSLRLSCAASGFEFRTYGMHWVRQAPGKGLEWVGVIWYDGSNTHYADSVKGRFTITRDNSKNTLNLQMNSLRVEDTAVYYCVRAPQWYLTAEAFDLWGQGTMVTVSS

SEQ ID NO: 262 - protein, 108 aa, synthetic construct
  REGION 1..108: note = Description of Artificial Sequence: Synthetic polypeptide
SYVLTQPPSVSVAPGETARITCGGNLIGARLVHWYQQKPGQAPVLVIYDDSDRPSRIPERFSGSNIGNTATLTIERVEEGDEADYYCQVWDYYSNSYVFGGGTKLTVL

SEQ ID NO: 263 - protein, 122 aa, synthetic construct; identical to SEQ ID NO: 239
  REGION 1..122: note = Description of Artificial Sequence: Synthetic polypeptide

SEQ ID NO: 264 - protein, 108 aa, synthetic construct
  REGION 1..108: note = Description of Artificial Sequence: Synthetic polypeptide
SYILTQPPSVSVAPGETATITCGGNLIGARLVHWYQQKPGQAPVLVIYDDSDRPSRIPERFSGSNIGNTATLTISRVEEGDEADYYCQVWDYSSNTYVFGGGTKLTVL

SEQ ID NO: 265 - protein, 122 aa, synthetic construct; identical to SEQ ID NO: 233
  REGION 1..122: note = Description of Artificial Sequence: Synthetic polypeptide

SEQ ID NO: 266 - protein, 108 aa, synthetic construct
  REGION 1..108: note = Description of Artificial Sequence: Synthetic polypeptide
SYVLTQPPSVSVAPGETATITCGGNLIGARLVHWYQQKPGQAPVLVVYDDSDRPSRIPERFSGSNIGNTATLTIEDVEAGDEADYYCQVWDYSSNTYVFGGGTKLTVL

SEQ ID NO: 267 - protein, 122 aa, synthetic construct; identical to SEQ ID NO: 261
  REGION 1..122: note = Description of Artificial Sequence: Synthetic polypeptide

SEQ ID NO: 268 - protein, 108 aa, synthetic construct
  REGION 1..108: note = Description of Artificial Sequence: Synthetic polypeptide
SYVLTQPPSVSVAPGQTATITCGGNLIGARLVHWYQQKPGQAPVLVVYDDSDRPSRIPERFSGSNIGNTATLTISDVEEGDEADYYCQVWDYYSNSYVFGGGTKLTVL

SEQ ID NO: 269 - protein, 122 aa, synthetic construct; identical to SEQ ID NO: 251
  REGION 1..122: note = Description of Artificial Sequence: Synthetic polypeptide

SEQ ID NO: 270 - protein, 108 aa, synthetic construct
  REGION 1..108: note = Description of Artificial Sequence: Synthetic polypeptide
SYILTQPPSVSVAPGETATITCGGNLIGARLVHWYQQKPGQAPVLVIYDDSDRPSRIPERFSGSNIGNTATLTIEDVEEGDEADYYCQVWDYYSDSYVFGGGTKLTVL

SEQ ID NO: 271 - protein, 122 aa, synthetic construct; identical to SEQ ID NO: 231
  REGION 1..122: note = Description of Artificial Sequence: Synthetic polypeptide

SEQ ID NO: 272 - protein, 108 aa, synthetic construct
  REGION 1..108: note = Description of Artificial Sequence: Synthetic polypeptide
SYVLTQPPSVSVAPGETATITCGGNLIGARLVHWYQQKPGQAPVLVIYDDSDRPSRIPERFSGSNIGNTATLTIERVEAGDEADYYCQVWDYYSNSYVFGGGTKLTVL

SEQ ID NO: 273 - protein, 122 aa, synthetic construct; identical to SEQ ID NO: 257
  REGION 1..122: note = Description of Artificial Sequence: Synthetic polypeptide

SEQ ID NO: 274 - protein, 108 aa, synthetic construct
  REGION 1..108: note = Description of Artificial Sequence: Synthetic polypeptide
SYILTQPPSVSVAPGQTATITCGGNLIGSRLVHWYQQKPGQAPVLVIYDDSDRPSRIPERFSGSNIGNTATLTIEDVEEGDEADYYCQVWDHYSDHMVFGGGTKLTVL

SEQ ID NO: 275 - protein, 122 aa, synthetic construct; identical to SEQ ID NO: 247
  REGION 1..122: note = Description of Artificial Sequence: Synthetic polypeptide

SEQ ID NO: 276 - protein, 108 aa, synthetic construct
  REGION 1..108: note = Description of Artificial Sequence: Synthetic polypeptide
SYILTQPPSVSVAPGQTATITCGGNLIGARLVHWYQQKPGQAPVLVIYDDSDRPSRIPERFSGSNIGNTATLTIERVEEGDEADYYCQVWDYSSDSYVFGGGTKLTVL

SEQ ID NO: 277 - protein, 122 aa, synthetic construct; identical to SEQ ID NO: 233
  REGION 1..122: note = Description of Artificial Sequence: Synthetic polypeptide

SEQ ID NO: 278 - protein, 108 aa, synthetic construct
  REGION 1..108: note = Description of Artificial Sequence: Synthetic polypeptide
SYILTQPPSVSVAPGETATITCGGNLIGARLVHWYQQKPGQAPVLVIYDDSDRPSRIPERFSGSNIGNTATLTISDVEEGDEADYYCQVWDYYSNSYVFGGGTKLTVL

SEQ ID NO: 279 - protein, 122 aa, synthetic construct; identical to SEQ ID NO: 233
  REGION 1..122: note = Description of Artificial Sequence: Synthetic polypeptide

SEQ ID NO: 280 - protein, 108 aa, synthetic construct
  REGION 1..108: note = Description of Artificial Sequence: Synthetic polypeptide
SYILTQPPSVSVAPGQTATITCGGNLIGARLVHWYQQKPGQAPVLVIYDDSDRPSRIPERFSGSNIGNTATLTIERVEEGDEADYYCQVWDYSSNSYVFGGGTKLTVL

SEQ ID NO: 281 - protein, 122 aa, synthetic construct
  REGION 1..122: note = Description of Artificial Sequence: Synthetic polypeptide
QMQLVESGGGVVQPGRSLRLSCAASGFEFRTYGMHWVRQAPGKGLEWVAVIWDDGSNTHYADSVKGRFTITRDNSKNTLNLQMNSLRAEDTAVYYCVRAPQWYLTAEAFDLWGQGTMVTVSS

SEQ ID NO: 282 - protein, 108 aa, synthetic construct
  REGION 1..108: note = Description of Artificial Sequence: Synthetic polypeptide
SYILTQPPSVSVAPGETTRITCGGNLIGAKLVHWYQQKPGQAPVLVIYDDSDRPSRIPERFSGSNIGNTATLTIEDVEAGDEADYYCQVWDYSSNHMVFGGGTKLTVL

SEQ ID NO: 283 - protein, 122 aa, synthetic construct
  REGION 1..122: note = Description of Artificial Sequence: Synthetic polypeptide
QMQLVESGGGVVQPGRSLTLSCAASGFEFRTYGMHWVRQAPGKGLEWVGVIWDDGSATHYADSVKGRFTITRDNSKNTLNLQMNSLRVEDTAVYYCVRAPQWYLTAEAFDLWGQGTMVTVSS

SEQ ID NO: 284 - protein, 108 aa, synthetic construct
  REGION 1..108: note = Description of Artificial Sequence: Synthetic polypeptide
SYVLTQPPSVSVAPGETATITCGGALIGARLVHWYQQKPGQAPVLVVYDDSDRPSRIPERFSGSNIGNTATLTISRVEAGDEADYYCQVWDYYANSYVFGGGTKLTVL

SEQ ID NO: 285 - protein, 122 aa, synthetic construct
  REGION 1..122: note = Description of Artificial Sequence: Synthetic polypeptide
QMQLVESGGGVVQPGRSLRLSCAASGFEFRTYGMHWVRQAPGKGLEWLGVIWYDGSNTHYADSVKGRFTITRDNSKNTLNLQMNSLRAEDTAVYYCVRAPQWYLTAEAFDLWGQGTMVTVSS

SEQ ID NO: 286 - protein, 108 aa, synthetic construct
  REGION 1..108: note = Description of Artificial Sequence: Synthetic polypeptide
SYILTQPPSVSVAPGQTATITCGGNLIGARLVHWYQQKPGQAPVLVIYDDSDRPSRIPERFSGSNIGNTATLTIERVEEGDEADYYCQVWDYSSDTYVFGGGTKLTVL

SEQ ID NO: 287 - protein, 122 aa, synthetic construct; identical to SEQ ID NO: 247
  REGION 1..122: note = Description of Artificial Sequence: Synthetic polypeptide

SEQ ID NO: 288 - protein, 108 aa, synthetic construct
  REGION 1..108: note = Description of Artificial Sequence: Synthetic polypeptide
SYVLTQPPSVSVAPGETATITCGGNLIGARLVHWYQQKPGQAPVLVIYDDSDRPSRIPERFSGSNIGNTATLTIEDVEEGDEADYYCQVWDYSSDSYVFGGGTKLTVL

SEQ ID NO: 289 - protein, 122 aa, synthetic construct; identical to SEQ ID NO: 231
  REGION 1..122: note = Description of Artificial Sequence: Synthetic polypeptide

SEQ ID NO: 290 - protein, 108 aa, synthetic construct
  REGION 1..108: note = Description of Artificial Sequence: Synthetic polypeptide
SYILTQPPSVSVAPGQTATITCGGNLIGARLVHWYQQKPGQAPVLVIYDDSDRPSRIPERFSGSNIGNTATLTIEDVEEGDEADYYCQVWDYSANSYVFGGGTKLTVL

SEQ ID NO: 291 - protein, 122 aa, synthetic construct
  REGION 1..122: note = Description of Artificial Sequence: Synthetic polypeptide
QMQLVESGGGVVQPGRSLTLSCAASGFEFRTYGMHWVRQAPGKGLEWLGVIWYDGSNTHYADSVKGRFTITRDNSKNTLNLQMNSLRAEDTAVYYCVRAPQWYLTAEAFDLWGQGTMVTVSS

SEQ ID NO: 292 - protein, 108 aa, synthetic construct
  REGION 1..108: note = Description of Artificial Sequence: Synthetic polypeptide
SYILTQPPSVSVAPGQTATITCGGNLIGARLVHWYQQKPGQAPVLVIYDDSDRPSRIPERFSGSNSGNTATLTISDVEEGDEADYYCQVWDYSSNTYVFGGGTKLTVL

SEQ ID NO: 293 - protein, 122 aa, synthetic construct
  REGION 1..122: note = Description of Artificial Sequence: Synthetic polypeptide
QMQLVESGGGVVQPGRSLTLSCAASGFEFRTYGMHWVRQAPGKGLEWVAVIWDDGSNTHYADSVKGRFTITRDNSKNTLNLQMNSLRVEDTAVYYCVRAPQWYLTAEAFDLWGQGTMVTVSS

SEQ ID NO: 294 - protein, 108 aa, synthetic construct
  REGION 1..108: note = Description of Artificial Sequence: Synthetic polypeptide
SYVLTQPPSVSVAPGQTATITCGGNLIGARLVHWYQQKPGQAPVLVIYDDSDRPSRIPERFSGSNIGNTATLTIERVEAGDEADYYCQVWDYYSDTMVFGGGTKLTVL

SEQ ID NO: 295 - protein, 122 aa, synthetic construct; identical to SEQ ID NO: 227
  REGION 1..122: note = Description of Artificial Sequence: Synthetic polypeptide

SEQ ID NO: 296 - protein, 108 aa, synthetic construct
  REGION 1..108: note = Description of Artificial Sequence: Synthetic polypeptide
SYVLTQPPSVSVAPGETATITCGGNLIGAKLVHWYQQKPGQAPVLVIYDDSDRPSRIPERFSGSNIGNTATLTIEDVEAGDEADYYCQVWDYSSDTMVFGGGTKLTVL

SEQ ID NO: 297 - protein, 122 aa, synthetic construct; identical to SEQ ID NO: 239
  REGION 1..122: note = Description of Artificial Sequence: Synthetic polypeptide

SEQ ID NO: 298 - protein, 108 aa, synthetic construct
  REGION 1..108: note = Description of Artificial Sequence: Synthetic polypeptide
SYILTQPPSVSVAPGETATITCGGNLIGARLVHWYQQKPGQAPVLVIYDDSDRPSRIPERFSGSNIGNTATLTISDVEEGDEADYYCQVWDYSSDHYVFGGGTKLTVL

SEQ ID NO: 299 - protein, 122 aa, synthetic construct; identical to SEQ ID NO: 233
  REGION 1..122: note = Description of Artificial Sequence: Synthetic polypeptide

SEQ ID NO: 300 - protein, 108 aa, synthetic construct; identical to SEQ ID NO: 264
  REGION 1..108: note = Description of Artificial Sequence: Synthetic polypeptide

SEQ ID NO: 301 - protein, 122 aa, synthetic construct; identical to SEQ ID NO: 281
  REGION 1..122: note = Description of Artificial Sequence: Synthetic polypeptide

SEQ ID NO: 302 - protein, 108 aa, synthetic construct
  REGION 1..108: note = Description of Artificial Sequence: Synthetic polypeptide
SYILTQPPSVSVAPGETATITCGGNLIGARLVHWYQQKPGQAPVLVIYDDSDRPSRIPERFSGSNSGNTATLTISDVEAGDEADYYCQVWDYSSDHMVFGGGTKLTVL

SEQ ID NO: 303 - protein, 122 aa, synthetic construct; identical to SEQ ID NO: 233
  REGION 1..122: note = Description of Artificial Sequence: Synthetic polypeptide

SEQ ID NO: 304 - protein, 108 aa, synthetic construct
  REGION 1..108: note = Description of Artificial Sequence: Synthetic polypeptide
SYILTQPPSVSVAPGETATITCGGNLIGARLVHWYQQKPGQAPVLVIYDDSDRPSRIPERFSGSNIGNTATLTISRVEAGDEADYYCQVWDYSSNTYVFGGGTKLTVL

SEQ ID NO: 305 - protein, 122 aa, synthetic construct; identical to SEQ ID NO: 281
  REGION 1..122: note = Description of Artificial Sequence: Synthetic polypeptide

SEQ ID NO: 306 - protein, 108 aa, synthetic construct
  REGION 1..108: note = Description of Artificial Sequence: Synthetic polypeptide
SYILTQPPSVSVAPGETATITCGGNLIGARLVHWYQQKPGQAPVLVIYDDSDRPSRIPERFSGSNIGNTATLTISRVEEGDEADYYCQVWDYSSDSYVFGGGTKLTVL

SEQ ID NO: 307 - protein, 122 aa, synthetic construct; identical to SEQ ID NO: 281
  REGION 1..122: note = Description of Artificial Sequence: Synthetic polypeptide

SEQ ID NO: 308 - protein, 108 aa, synthetic construct
  REGION 1..108: note = Description of Artificial Sequence: Synthetic polypeptide
SYILTQPPSVSVAPGETATITCGGNLIGARLVHWYQQKPGQAPVLVIYDDSDRPSRIPERFSGSNIGNTATLTISDVEAGDEADYYCQVWDYSSDTYVFGGGTKLTVL

SEQ ID NO: 309 - protein, 122 aa, synthetic construct
  REGION 1..122: note = Description of Artificial Sequence: Synthetic polypeptide
QMQLVESGGGVVQPGRSLRLSCAASGFEFRTYGMHWVRQAPGKGLEWVGVIWDDGSNTHYADSVKGRFTITRDNSKNTLNLQMNSLRAEDTAVYYCVRAPQWYLTAEAFDLWGQGTMVTVSS

SEQ ID NO: 310 - protein, 108 aa, synthetic construct; identical to SEQ ID NO: 308
  REGION 1..108: note = Description of Artificial Sequence: Synthetic polypeptide

SEQ ID NO: 311 - protein, 122 aa, synthetic construct; identical to SEQ ID NO: 281
  REGION 1..122: note = Description of Artificial Sequence: Synthetic polypeptide

SEQ ID NO: 312 - protein, 108 aa, synthetic construct
  REGION 1..108: note = Description of Artificial Sequence: Synthetic polypeptide
SYVLTQPPSVSVAPGETATITCGGNLIGARLVHWYQQKPGQAPVLVIYDDSDRPSRIPERFSGSNIGNTATLTIERVEEGDEADYYCQVWDYSSDHMVFGGGTKLTVL

SEQ ID NO: 313 - protein, 122 aa, synthetic construct; identical to SEQ ID NO: 309
  REGION 1..122: note = Description of Artificial Sequence: Synthetic polypeptide

SEQ ID NO: 314 - protein, 108 aa, synthetic construct
  REGION 1..108: note = Description of Artificial Sequence: Synthetic polypeptide
SYILTQPPSVSVAPGQTARITCGGNLIGAKLVHWYQQKPGQAPVLVIYDDSDRPSRIPERFSGSNIGNTATLTIERVEEGDEADYYCQVWDYSSDHMVFGGGTKLTVL

SEQ ID NO: 315 - protein, 122 aa, synthetic construct
  REGION 1..122: note = Description of Artificial Sequence: Synthetic polypeptide
QMQLVESGGGVVQPGRSLTLSCAASGFEFRTYGMHWVRQAPGKGLEWVGVIWYDGSATHYADSVKGRFTITRDNSKNTLNLQMNSLRAEDTAVYYCARAPQWYLTAEAFDLWGQGTMVTVSS

SEQ ID NO: 316 - protein, 108 aa, synthetic construct
  REGION 1..108: note = Description of Artificial Sequence: Synthetic polypeptide
SYILTQPPSVSVAPGETATITCGGNLIGAKLVHWYQQKPGQAPVLVIYDDSDRPSRIPERFSGSNIGNTATLTISDVEAGDEADYYCQVWDYYSDTYVFGGGTKLTVL

SEQ ID NO: 317 - protein, 122 aa, synthetic construct; identical to SEQ ID NO: 293
  REGION 1..122: note = Description of Artificial Sequence: Synthetic polypeptide

SEQ ID NO: 318 - protein, 108 aa, synthetic construct
  REGION 1..108: note = Description of Artificial Sequence: Synthetic polypeptide
SYILTQPPSVSVAPGETATITCGGNLIGAKLVHWYQQKPGQAPVLVIYDDSDRPSRIPERFSGSNSGNTATLTISDVEEGDEADYYCQVWDYYADTMVFGGGTKLTVL

SEQ ID NO: 319 - protein, 122 aa, synthetic construct
  REGION 1..122: note = Description of Artificial Sequence: Synthetic polypeptide
QMQLVESGGGVVQPGRSLTLSCAASGFEFRTYGMHWVRQAPGKGLEWVGVIWDDGSNTHYADSVKGRFTITRDNSKNTLNLQMNSLRVEDTAVYYCARAPQWYLTAEAFDLWGQGTMVTVSS

SEQ ID NO: 320 - protein, 108 aa, synthetic construct
  REGION 1..108: note = Description of Artificial Sequence: Synthetic polypeptide
SYILTQPPSVSVAPGQTATITCGGNLIGAKLVHWYQQKPGQAPVLVIYDDSDRPSRIPERFSGSNIGNTATLTISDVEEGDEADYYCQVWDYSSDHMVFGGGTKLTVL

SEQ ID NO: 321 - protein, 122 aa, synthetic construct
  REGION 1..122: note = Description of Artificial Sequence: Synthetic polypeptide
QMQLVESGGGVVQPGRSLTLSCAASGFAFRTYGMHWVRQAPGKGLEWVAVIWDDGSNTHYADSVKGRFTITRDNSKNTLNLQMNSLRVEDTAVYYCVRAPQWYLTAEAFDLWGQGTMVTVSS

SEQ ID NO: 322 - protein, 108 aa, synthetic construct
  REGION 1..108: note = Description of Artificial Sequence: Synthetic polypeptide
SYILTQPPSVSVAPGETATITCGGNLIGARLVHWYQQKPGQAPVLVIYDDSDRPSRIPERFSGSNIGNTATLTIERVEAGDEADYYCQVWDYSSNSYVFGGGTKLTVL

SEQ ID NO: 323 - protein, 122 aa, synthetic construct
  REGION 1..122: note = Description of Artificial Sequence: Synthetic polypeptide
QMQLVESGGGVVQPGRSLTLSCAASGFEFRTYGMHWVRQAPGKGLEWVAVIWDDGSNTHYADSVKGRFTITRDNSKNTLNLQMNSLRAEDTAVYYCVRAPQWYLTAEAFDLWGQGTMVTVSS

SEQ ID NO: 324 - protein, 108 aa, synthetic construct
  REGION 1..108: note = Description of Artificial Sequence: Synthetic polypeptide
SYILTQPPSVSVAPGETATITCGGNLIGAKLVHWYQQKPGQAPVLVIYDDSDRPSRIPERFSGSNIGNTATLTISDVEEGDEADYYCQVWDYYSNTMVFGGGTKLTVL

SEQ ID NO: 325 - protein, 122 aa, synthetic construct; identical to SEQ ID NO: 309
  REGION 1..122: note = Description of Artificial Sequence: Synthetic polypeptide

SEQ ID NO: 326 - protein, 108 aa, synthetic construct
  REGION 1..108: note = Description of Artificial Sequence: Synthetic polypeptide
SYILTQPPSVSVAPGETATITCGGNLIGARLVHWYQQKPGQAPVLVIYDDSDRPSRIPERFSGSNIGNTATLTISRVEAGDEADYYCQVWDYSSDHMVFGGGTKLTVL

SEQ ID NO: 327 - protein, 122 aa, synthetic construct; identical to SEQ ID NO: 281
  REGION 1..122: note = Description of Artificial Sequence: Synthetic polypeptide

SEQ ID NO: 328 - protein, 108 aa, synthetic construct
  REGION 1..108: note = Description of Artificial Sequence: Synthetic polypeptide
SYILTQPPSVSVAPGETATITCGGNLIGARLVHWYQQKPGQAPVLVIYDDSDRPSRIPERFSGSNIGNTATLTISRVEAGDEADYYCQVWDYSSDTYVFGGGTKLTVL

SEQ ID NO: 329 - protein, 122 aa, synthetic construct
  REGION 1..122: note = Description of Artificial Sequence: Synthetic polypeptide
QMQLVESGGGVVQPGQSLRLSCAASGFEFRTYGMHWVRQAPGKGLEWLAVIWYDGSNTHYADSVKGRFTITRDNSKNTLNLQMNSLRAEDTAVYYCVRAPQWYNSAEAFDLWGQGTMVTVSS

SEQ ID NO: 330 - protein, 108 aa, synthetic construct
  REGION 1..108: note = Description of Artificial Sequence: Synthetic polypeptide
SYVLTQPPSVSVAPGQTARITCGGNLIGARLVHWYQQKPGQAPVLVIYDDSDRPSHIPERFSGSNSGNTATLTISRVEAGDEADYYCQVWDYSADTMVFGGGTKLTVL

SEQ ID NO: 331 - protein, 122 aa, synthetic construct
  REGION 1..122: note = Description of Artificial Sequence: Synthetic polypeptide
QMQLVESGGGVVQPGQSLRLSCAASGFAFRTYGMHWVRQAPGKGLEWLAVIWYDGSNTHYADSVKGRFTITRDNSKNTLNLQMNSLRAEDTAVYYCVRAPQWYNSAEAFDLWGQGTMVTVSS

SEQ ID NO: 332 - protein, 108 aa, synthetic construct
  REGION 1..108: note = Description of Artificial Sequence: Synthetic polypeptide
SYVLTQPPSVSVAPGQTARITCGGNLIGARLVHWYQQKPGQAPVLVVYDDSDRPSHIPERFSGSNSGNTATLTISRVEAGDEADYYCQVWDHSADTMVFGGGTKLTVL

SEQ ID NO: 333 - protein, 122 aa, synthetic construct
  REGION 1..122: note = Description of Artificial Sequence: Synthetic polypeptide
QMQLVESGGGVVQPGRSLRLSCAASGFEFRTYGMHWVRQAPGKGLEWLAVIWYDGSNTHYADSVKGRFTITRDNSKNTLNLQMNSLRAEDTAVYYCVRAPQWYNSAEAFDLWGQGTMVTVSS

SEQ ID NO: 334 - protein, 108 aa, synthetic construct
  REGION 1..108: note = Description of Artificial Sequence: Synthetic polypeptide
SYVLTQPPSVSVAPGQTARITCGGNLIGAKLVHWYQQKPGQAPVLVIYDDSDRPSHIPERFSGSNSGNTATLTISRVEAGDEADYYCQVWDYSSDTMVFGGGTKLTVL

SEQ ID NO: 335 - protein, 122 aa, synthetic construct
  REGION 1..122: note = Description of Artificial Sequence: Synthetic polypeptide
QMQLVESGGGVVQPGQSLRLSCAASGFEFRTYGMHWVRQAPGKGLEWVAVIWDDGSNTHYADSVKGRFTITRDNSKNTLNLQMNSLRAEDTAVYYCVRAPQWYLTAEAFDLWGQGTMVTVSS

SEQ ID NO: 336 - protein, 108 aa, synthetic construct
  REGION 1..108: note = Description of Artificial Sequence: Synthetic polypeptide
SYVLTQPPSVSVAPGETATITCGGNLIGARLVHWYQQKPGQAPVLVIYDDSDRPSHIPERFSGSNSGNTATLTISRVEAGDEADYYCQVWDYSADTMVFGGGTKLTVL

SEQ ID NO: 337 - protein, 122 aa, synthetic construct; identical to SEQ ID NO: 209
  REGION 1..122: note = Description of Artificial Sequence: Synthetic polypeptide

SEQ ID NO: 338 - protein, 108 aa, synthetic construct
  REGION 1..108: note = Description of Artificial Sequence: Synthetic polypeptide
SYVLTQPPSVSVAPGETASITCGGNMIGGYLVHWYQQKPGQAPVLVIYDDVDRPSRIPERFSGSNSGNTATLTISRVEAGDEADYYCQVWDHNSNHMVFGGGTKLTVL

SEQ ID NO: 339 - protein, 122 aa, synthetic construct
  REGION 1..122: note = Description of Artificial Sequence: Synthetic polypeptide
QMQLVESGGGVVQPGRSLRLSCAASGFAFRTYGMHWVRQAPGKGLEWVAVIWDDGSNTHYADSVKGRFTITRDNSKNTLNLQMNSLRAEDTAVYYCVRAPQWYLSAEAFDLWGQGTMVTVSS

SEQ ID NO: 340 - protein, 108 aa, synthetic construct
  REGION 1..108: note = Description of Artificial Sequence: Synthetic polypeptide
SYVLTQPPSVSVAPGETATITCGGNMIGAYLVHWYQQKPGQAPLLVIYDDVDRPARIPERFSGSNSGNTATLTISRVEAGDEADYYCQVWDHNSDHMVFGGGTKLTVL

SEQ ID NO: 341 - protein, 122 aa, synthetic construct; identical to SEQ ID NO: 319
  REGION 1..122: note = Description of Artificial Sequence: Synthetic polypeptide

SEQ ID NO: 342 - protein, 108 aa, synthetic construct
  REGION 1..108: note = Description of Artificial Sequence: Synthetic polypeptide
SYILTQPPSVSVAPGETATITCGGNLIGAKLVHWYQQKPGQAPVLVIYDDSDRPSRIPERFSGSNIGNTATLTIEDVEAGDEADYYCQVWDYSANHMVFGGGTKLTVL

SEQ ID NO: 343 - protein, 122 aa, synthetic construct; identical to SEQ ID NO: 281
  REGION 1..122: note = Description of Artificial Sequence: Synthetic polypeptide

SEQ ID NO: 344 - protein, 108 aa, synthetic construct
  REGION 1..108: note = Description of Artificial Sequence: Synthetic polypeptide
SYILTQPPSVSVAPGETATITCGGALIGSRLVHWYQQKPGQAPVLVIYDDSDRPSRIPERFSGSNIGNTATLTISDVEEGDEADYYCQVWDYYSNHMVFGGGTKLTVL

SEQ ID NO: 345 - protein, 122 aa, synthetic construct; identical to SEQ ID NO: 323
  REGION 1..122: note = Description of Artificial Sequence: Synthetic polypeptide

SEQ ID NO: 346 - protein, 108 aa, synthetic construct
  REGION 1..108: note = Description of Artificial Sequence: Synthetic polypeptide
SYILTQPPSVSVAPGQTATITCGGNLIGAKLVHWYQQKPGQAPVLVVYDDSDRPSRIPERFSGSNIGNTATLTIERVEEGDEADYYCQVWDYYSHTMVFGGGTKLTVL

SEQ ID NO: 347 - protein, 122 aa, synthetic construct
  REGION 1..122: note = Description of Artificial Sequence: Synthetic polypeptide
QMQLVESGGGVVQPGRSLRLSCAASGFEFRTYGMHWVRQAPGKGLEWVGVIWDDGSATHYADSVKGRFTITRDNSKNTLNLQMNSLRVEDTAVYYCVRAPQWYLTAEAFDLWGQGTMVTVSS

SEQ ID NO: 348 - protein, 108 aa, synthetic construct
  REGION 1..108: note = Description of Artificial Sequence: Synthetic polypeptide
SYILTQPPSVSVAPGQTATITCGGALIGARLVHWYQQKPGQAPVLVIYDDSDRPSRIPERFSGSNIGNTATLTISRVEEGDEADYYCQVWDYSSNTYVFGGGTKLTVL

SEQ ID NO: 349 - protein, 8 aa, synthetic construct
  REGION 1..8: note = Description of Artificial Sequence: Synthetic peptide
GFAFRTYG

SEQ ID NO: 350 - protein, 8 aa, synthetic construct
  REGION 1..8: note = Description of Artificial Sequence: Synthetic peptide
IWDDGSNT

SEQ ID NO: 351 - protein, 15 aa, synthetic construct
  REGION 1..15: note = Description of Artificial Sequence: Synthetic peptide
VRAPQWYLTAEAFDL

SEQ ID NO: 352 - protein, 8 aa, synthetic construct
  REGION 1..8: note = Description of Artificial Sequence: Synthetic peptide
IWYDGSAT

SEQ ID NO: 353 - protein, 15 aa, synthetic construct
  REGION 1..15: note = Description of Artificial Sequence: Synthetic peptide
VRAPQWYLSAEAFDL

SEQ ID NO: 354 - protein, 8 aa, synthetic construct
  REGION 1..8: note = Description of Artificial Sequence: Synthetic peptide
IWDDGSAT

SEQ ID NO: 355 - protein, 8 aa, synthetic construct
  REGION 1..8: note = Description of Artificial Sequence: Synthetic peptide
GFEFRTYG

SEQ ID NO: 356 - protein, 8 aa, synthetic construct
  REGION 1..8: note = Description of Artificial Sequence: Synthetic peptide
IWYDGSNT

SEQ ID NO: 357 - protein, 15 aa, synthetic construct
  REGION 1..15: note = Description of Artificial Sequence: Synthetic peptide
ARAPQWYLTAEAFDL

SEQ ID NO: 358 - protein, 15 aa, synthetic construct
  REGION 1..15: note = Description of Artificial Sequence: Synthetic peptide
VRAPQWYNSAEAFDL

SEQ ID NO: 359 - protein, 6 aa, synthetic construct
  REGION 1..6: note = Description of Artificial Sequence: Synthetic peptide
MIGAYL

SEQ ID NO: 360 - skipped

SEQ ID NO: 361 - protein, 11 aa, synthetic construct
  REGION 1..11: note = Description of Artificial Sequence: Synthetic peptide
QVWDHNTDKMV

SEQ ID NO: 362 - protein, 6 aa, synthetic construct
  REGION 1..6: note = Description of Artificial Sequence: Synthetic peptide
LIGAYL

SEQ ID NO: 363 - protein, 11 aa, synthetic construct
  REGION 1..11: note = Description of Artificial Sequence: Synthetic peptide
QVWDHDTNTMV

SEQ ID NO: 364 - protein, 6 aa, synthetic construct
  REGION 1..6: note = Description of Artificial Sequence: Synthetic peptide
MIGGYL

SEQ ID NO: 365 - protein, 11 aa, synthetic construct
  REGION 1..11: note = Description of Artificial Sequence: Synthetic peptide
QVWDHDTNHMV

SEQ ID NO: 366 - protein, 6 aa, synthetic construct
  REGION 1..6: note = Description of Artificial Sequence: Synthetic peptide
LIGSRL

SEQ ID NO: 367 - skipped

SEQ ID NO: 368 - protein, 11 aa, synthetic construct
  REGION 1..11: note = Description of Artificial Sequence: Synthetic peptide
QVWDHSSNTYV

SEQ ID NO: 369 - protein, 6 aa, synthetic construct
  REGION 1..6: note = Description of Artificial Sequence: Synthetic peptide
LIGARL

SEQ ID NO: 370 - protein, 11 aa, synthetic construct
  REGION 1..11: note = Description of Artificial Sequence: Synthetic peptide
QVWDYYSDHMV

SEQ ID NO: 371 - protein, 11 aa, synthetic construct
  REGION 1..11: note = Description of Artificial Sequence: Synthetic peptide
QVWDHDSNTMV

SEQ ID NO: 372 - protein, 11 aa, synthetic construct
  REGION 1..11: note = Description of Artificial Sequence: Synthetic peptide
QVWDHNTNHMV

SEQ ID NO: 373 - protein, 11 aa, synthetic construct
  REGION 1..11: note = Description of Artificial Sequence: Synthetic peptide
QVWDHNTQQMV

SEQ ID NO: 374 - protein, 11 aa, synthetic construct
  REGION 1..11: note = Description of Artificial Sequence: Synthetic peptide
QVWDHDTNQVV

SEQ ID NO: 375 - protein, 6 aa, synthetic construct
  REGION 1..6: note = Description of Artificial Sequence: Synthetic peptide
LIGAKL

SEQ ID NO: 376 - protein, 11 aa, synthetic construct
  REGION 1..11: note = Description of Artificial Sequence: Synthetic peptide
QVWDYSSDTMV

SEQ ID NO: 377 - protein, 11 aa, synthetic construct
  REGION 1..11: note = Description of Artificial Sequence: Synthetic peptide
QVWDHSTNTMV

SEQ ID NO: 378 - protein, 11 aa, synthetic construct
  REGION 1..11: note = Description of Artificial Sequence: Synthetic peptide
QVWDYSSNSYV

SEQ ID NO: 379 - protein, 11 aa, synthetic construct
  REGION 1..11: note = Description of Artificial Sequence: Synthetic peptide
QVWDYSSNTYV

SEQ ID NO: 380 - protein, 11 aa, synthetic construct
  REGION 1..11: note = Description of Artificial Sequence: Synthetic peptide
QVWDHNSNQMV

SEQ ID NO: 381 - protein, 11 aa, synthetic construct
  REGION 1..11: note = Description of Artificial Sequence: Synthetic peptide
QVWDHSSNHYV

SEQ ID NO: 382 - protein, 11 aa, synthetic construct
  REGION 1..11: note = Description of Artificial Sequence: Synthetic peptide
QVWDHYSNHYV

SEQ ID NO: 383 - protein, 11 aa, synthetic construct
  REGION 1..11: note = Description of Artificial Sequence: Synthetic peptide
QVWDYSADSYV

SEQ ID NO: 384 - protein, 11 aa, synthetic construct
  REGION 1..11: note = Description of Artificial Sequence: Synthetic peptide
QVWDHNSNHMV

SEQ ID NO: 385 - protein, 11 aa, synthetic construct
  REGION 1..11: note = Description of Artificial Sequence: Synthetic peptide
QVWDYYSDSYV

SEQ ID NO: 386 - protein, 11 aa, synthetic construct
  REGION 1..11: note = Description of Artificial Sequence: Synthetic peptide
QVWDYSSDSMV

SEQ ID NO: 387 - protein, 11 aa, synthetic construct
  REGION 1..11: note = Description of Artificial Sequence: Synthetic peptide
QVWDYYSDHYV

SEQ ID NO: 388 - protein, 11 aa, synthetic construct
  REGION 1..11: note = Description of Artificial Sequence: Synthetic peptide
QVWDYSSDSYV

SEQ ID NO: 389 - protein, 11 aa, synthetic construct
  REGION 1..11: note = Description of Artificial Sequence: Synthetic peptide
QVWDYYSNSYV

SEQ ID NO: 390 - protein, 11 aa, synthetic construct
  REGION 1..11: note = Description of Artificial Sequence: Synthetic peptide
QVWDYYSDSYV

SEQ ID NO: 391 - protein, 11 aa, synthetic construct
  REGION 1..11: note = Description of Artificial Sequence: Synthetic peptide
QVWDHYSDHMV

SEQ ID NO: 392 - protein, 11 aa, synthetic construct
  REGION 1..11: note = Description of Artificial Sequence: Synthetic peptide
QVWDYSSNHMV

SEQ ID NO: 393 - protein, 11 aa, synthetic construct
  REGION 1..11: note = Description of Artificial Sequence: Synthetic peptide
QVWDYYANSYV

SEQ ID NO: 394 - protein, 11 aa, synthetic construct
  REGION 1..11: note = Description of Artificial Sequence: Synthetic peptide
QVWDYSSDTYV

SEQ ID NO: 395 - protein, 11 aa, synthetic construct
  REGION 1..11: note = Description of Artificial Sequence: Synthetic peptide
QVWDYSANSYV

SEQ ID NO: 396 - protein, 11 aa, synthetic construct
  REGION 1..11: note = Description of Artificial Sequence: Synthetic peptide
QVWDYYSDTMV

SEQ ID NO: 397 - protein, 11 aa, synthetic construct
  REGION 1..11: note = Description of Artificial Sequence: Synthetic peptide
QVWDYSSDHYV

SEQ ID NO: 398 - protein, 11 aa, synthetic construct
  REGION 1..11: note = Description of Artificial Sequence: Synthetic peptide
QVWDYSSDHMV

SEQ ID NO: 399 - protein, 11 aa, synthetic construct
  REGION 1..11: note = Description of Artificial Sequence: Synthetic peptide
QVWDYYSDTYV

SEQ ID NO: 400 - protein, 11 aa, synthetic construct
  REGION 1..11: note = Description of Artificial Sequence: Synthetic peptide
QVWDYYADTMV

SEQ ID NO: 401 - protein, 11 aa, synthetic construct
  REGION 1..11: note = Description of Artificial Sequence: Synthetic peptide
QVWDYYSNTMV

SEQ ID NO: 402 - protein, 11 aa, synthetic construct
  REGION 1..11: note = Description of Artificial Sequence: Synthetic peptide
QVWDYSADTMV

SEQ ID NO: 403 - protein, 11 aa, synthetic construct
  REGION 1..11: note = Description of Artificial Sequence: Synthetic peptide
QVWDHSADTMV

SEQ ID NO: 404 - protein, 11 aa, synthetic construct
  REGION 1..11: note = Description of Artificial Sequence: Synthetic peptide
QVWDHNSDHMV

SEQ ID NO: 405 - protein, 11 aa, synthetic construct
  REGION 1..11: note = Description of Artificial Sequence: Synthetic peptide
QVWDYSANHMV

SEQ ID NO: 406 - protein, 11 aa, synthetic construct
  REGION 1..11: note = Description of Artificial Sequence: Synthetic peptide
QVWDYYSNHMV

SEQ ID NO: 407 - protein, 11 aa, synthetic construct
  REGION 1..11: note = Description of Artificial Sequence: Synthetic peptide
QVWDYYSHTMV

SEQ ID NO: 408 - protein, 452 aa, synthetic construct
QMQLVESGGGVVQPGRSLRLSCAASGFAFRTYGMHWVRQAPGKGLEWVAVIWDDGSNTHYADSVKGRFTITRDNSKNTLNLQMNSLRAEDTAVYYCVRAPQWYLTAEAFDLWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 409 - protein, 214 aa, synthetic construct
SYVLTQPPSVSVAPGETASITCGGNMIGGYLVHWYQQKPGQAPVLVIYDDVDRPSRIPERFSGSNSGNTATLTISRVEAGDEADYYCQVWDHNSNHMVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 410 - protein, 452 aa, synthetic construct
QMQLVESGGGVVQPGRSLRLSCAASGFAFRTYGMHWVRQAPGKGLEWVAVIWDDGSNTHYADSVKGRFTITRDNSKNTLNLQMNSLRAEDTAVYYCVRAPQWYLTAEAFDLWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2024/0409625 A9
(43) Pub. Date: Dec. 12, 2024

(54) ENGINEERED DUAL BINDING ANTIBODIES AND USES THEREOF

(71) Applicant: Biolojic Design Ltd., Rehovot (IL)

(72) Inventors: ALIK DEMISHTEIN, Rosh Haayin (IL); Shmuel BERNSTEIN, Lod (IL); Tomer SHLAMKOVICH, Modi'in-Maccabim-Re'ut (IL); Ayelet CHEN, Jerusalem (IL); Yehezkel SASSON, Tzur Yigal (IL); Marek STRAJBL, Jerusalem (IL); Itay LEVIN, Herzeliya (IL); Sharon FISCHMAN, Modi'in (IL); Yanay OFRAN, Tel Aviv (IL); Guy NIMROD, Tel Aviv (IL); Alexey SHNYDER, Petah-Tiqva (IL); Hadar GATTEGNO, Rehovot (IL); Nikol MALCHENKO, Ramat Gan (IL); Olga BLUVSHTEIN YERMOLAEV, Rishon-LeZion (IL); Noam GROSSMAN, Mazkeret Batyia (IL); Liron DANIELPUR, Hadera (IL); Itzhak MEIR, Rehovot (IL); Morya IFRACH, Qiryat Gat (IL); Reut BARAK FUCHS, Rehovot (IL); Michael ZHENIN, Jerusalem (IL)

(73) Assignee: Biolojic Design Ltd., Rehovot (IL)

(21) Appl. No.: 18/475,358

(22) Filed: Sep. 27, 2023

Related U.S. Application Data
(63) Continuation-in-part of application No. PCT/IL2022/050572, filed on May 29, 2022.
Application No. PCT/IL2022/050572 is a continuation-in-part of application No. PCT/IL2022/050087, filed on Jan. 20, 2022.
(60) Provisional application No. 63/195,021, filed on May 30, 2021.
(60) Provisional application No. 63/295,905, filed on Jan. 2, 2022.

(51) Int. Cl. C07K 16/24

(52) U.S. Cl. CPC C07K 16/244; C07K 2317/31; C07K 2317/33; C07K 2317/52; C07K 2317/622; C07K 2317/732; C07K 2317/76; C07K 2317/92

(57) ABSTRACT

Described herein are engineered dual binding antibodies that bind to IL-13 and TSLP and uses thereof. Uses include treating allergic and respiratory conditions. Described herein are also libraries comprising the engineered dual binding antibodies, and methods of producing the engineered dual binding antibodies and functional and biochemical characterization of the antibodies.

### CROSS-REFERENCE TO RELATED APPLICATIONS

This application is a continuation-in-part application of PCT International Application No. PCT/IL2022/050572, International filing date May 29, 2022, which published as WO 2022/254428 on Dec. 8, 2022, claiming the benefit of PCT International Application No. PCT/IL2022/050087, filed Jan. 20, 2022, and U.S. Patent Applications Nos. 63/195,021, filed May 30, 2021, and 63/295,905, filed Jan. 2, 2022, which are all hereby incorporated by reference.

### SEQUENCE LISTING STATEMENT

The instant application contains a Sequence Listing which has been submitted electronically in XML file format and is hereby incorporated by reference in its entirety. Said XML copy, created on Aug. 16, 2023, is named P-605548-US_SL.xml and is 597,438 bytes in size.

### FIELD OF INTEREST

The disclosure relates in general to dual binding antibodies that bind to IL-13 and TSLP. In one embodiment, the antibodies can be used for treating allergic or respiratory conditions.

### BACKGROUND

Interleukin 13 (IL-13) is a 12.3 kDa monomeric protein of the class I cytokines. IL-13 has a 4 alpha helical bundle core topology typical of class I short helical cytokines; it is similar in structure to its closely related cytokine IL-4 sharing low sequence identity but high structural identity. Along with IL-4, IL-13 has been shown to control immunoglobulin class switching to IgE in B cells and is involved in mast cell recruitment. IL-13 is secreted by CD4⁺ Th2 cells, as well as type 2 innate lymphoid cells ILC2. It has been demonstrated that IL-13 can trigger the production of TGF-β and in bronchial epithelial cells induces gene expression of MUC5AC and production of mucin. IL-13 can also enhance contraction in smooth bronchial muscle cells. IL-13 binds the IL-4Ra/IL-13Ra1 heterodimeric complex, and upon binding it triggers a JAK-signal transducer and STATE dependent signaling cascade, which in turn triggers Th2 helper T-cell differentiation, polarization of macrophages to the M2 “alternatively activated” phenotype, epithelial mucus production, smooth muscle contractility and chemokine release.

IL-13 has been shown to be involved in protection against parasites, wherein it was demonstrated that in knockout IL-13 mice model, clearance of N. brasiliensis is severely delayed. Also, the expulsion of Trichuris muris is abolished completely, in spite of an otherwise intact Th2-type response. Further research demonstrated that IL-13 is a double-edged sword, on the one hand it has a major role in protection against parasites, however IL-13 function in situations of dysregulated immune system is also known.

IL-13 has been implicated in the pathogenesis of human asthma as elevated levels of IL-13 mRNA and protein have been detected in lungs of asthmatic patients, which correlate with severity of the disease. In addition, human IL-13 genetic polymorphisms, which lead to elevated IL-13 levels, have been identified and are associated with asthma and atopy, and elevated IL-13 levels have been detected in the lung of asthma patients.

Although IL-13 and IL-4 share similar receptors and signaling pathway, IL-13 has distinguished role in asthma, which is independent of IL-4. It has been shown in mice models that administration of IL-13 alone is sufficient to induce eosinophil derived inflammation, and mucus cell hyperplasia. Moreover, a specific blockade of IL-13 but not IL-4 is sufficient to reverse airway hyperreactivity and mucus production in mice models. Additionally, polymorphism in the human IL-13 locus is known to be associated with high susceptibility for asthma.

Specific inhibition of IL-13 signaling could therefore have positive therapeutic effect on asthma patients, or patients with other known allergic or respiratory conditions.

Thymic stromal lymphopoietin (TSLP) is a cytokine that signals through a heterodimeric receptor consisting of the IL-7Ra subunit and TSLP-R, a unique component with homology to the common γ-receptor-like chain. TSLP is expressed by epithelial cells in the thymus, lung, skin, intestine, and tonsils, as well as airway smooth muscle cells, lung fibroblasts, and stromal cells. These cells produce TSLP in response to proinflammatory stimuli, and TSLP drives allergic inflammatory responses through its activity on a number of innate immune cells, including dendritic cells. TSLP can also promote proliferation of naive T cells and drive their differentiation into Th2 cells expressing high levels of IL-4, IL-5, and IL-13. High level of TSLP expression has been found in asthmatic lung epithelial cells and chronic atopic dermatitis lesions, suggesting a role for TSLP in allergic inflammation. Recent evidence also implicates TSLP in the differentiation of Th17 cells and Th17-driven inflammatory processes. Chronic allergic (atopic) asthma is often characterized by Th2-type inflammation, while non-allergic asthmatic inflammation is predominately neutrophilic with a mixed Th1 and Th17 cytokine milieu. Antagonists to TSLP would be expected to be useful for treating inflammatory conditions.

Thus, there remains an unmet need for compositions and methods of treatment of diseases and conditions triggered by IL-13 and TSLP activation, for example but not limited to allergic and respiratory conditions, including but not limited to asthma.

### SUMMARY

In one embodiment, the present disclosure provides an isolated dual binding antibody comprising three complementarity determining regions (CDRs) on a heavy chain (HCDR1, HCDR2, and HCDR3) and three CDRs on a light chain (LCDR1, LCDR2, and LCDR3), wherein the CDRs have the sequences of SEQ ID NOs: 149-154. In another embodiment, the dual binding antibody comprises a heavy chain variable domain (VH) and a light chain variable domain (VL) having the amino acid sequences of SEQ ID Nos:155 and 156, or SEQ ID Nos:157 and 158.

Disclosed herein, in one aspect is an isolated dual binding antibody, wherein the HCDR1, HCDR2 and HCDR3 comprise the amino acid sequence of SEQ ID NOs:349, 350 and 351 respectively, and the LCDR1, LCDR2 and LCDR3 comprise the amino acid sequence of SEQ ID NO: 359, D D V, and SEQ ID NO: 361 respectively.

In another embodiment, the HCDR1, HCDR2 and HCDR3 comprise the amino acid sequence of SEQ ID NOs:349, 356 and 351 respectively, and the LCDR1, LCDR2 and LCDR3 comprise the amino acid sequence of SEQ ID NO: 364, D D V, and SEQ ID NO: 371 respectively.

In another embodiment, the HCDR1, HCDR2 and HCDR3 comprise the amino acid sequence of SEQ ID NOs:349, 350 and 351 respectively, and the LCDR1, LCDR2 and LCDR3 comprise the amino acid sequence of SEQ ID NO: 362, D D V, and SEQ ID NO: 384 respectively.

In another embodiment, the HCDR1, HCDR2 and HCDR3 comprise the amino acid sequence of SEQ ID NOs:349, 350 and 351 respectively, and the LCDR1, LCDR2 and LCDR3 comprise the amino acid sequence of SEQ ID NO: 364, D D V, and SEQ ID NO: 384 respectively.

In another embodiment, the HCDR1, HCDR2 and HCDR3 comprise the amino acid sequences as shown in Table 8 or Table 4, wherein the LCDR1, LCDR2 and LCDR3 comprise the amino acid sequences as shown in Table 9 or Table 5.

Disclosed herein, in one aspect is an isolated dual binding antibody, said dual binding antibody comprising an antibody antigen-binding domain site comprising a heavy chain variable region (VH) domain and a light chain variable region (VL) domain, wherein said VH domain comprises a set of complementarity-determining regions (CDRs), HCDR1, HCDR2, and HCDR3, wherein the amino acid sequence of HCDR1 is set forth in SEQ ID NO: 136; wherein the amino acid sequence of HCDR2 is set forth as: I HX1 Y D G S N K (SEQ ID NO: 142), wherein HX1 is any amino acid; and wherein the amino acid sequence of HCDR3 is set forth as: A R HX2 HX3 HX4 HX5 HX6 HX7 HX8 HX9 HX10 HX11 F D HX12 (SEQ ID NO: 143), wherein XH2, HX3, HX4, HX5, HX6, HX7, HX8, HX9, HX10, HX11, and HX12 are any amino acid; or wherein said VL domain comprises a set of CDRs, LCDR1, LCDR2, and LCDR3, wherein the amino acid sequence of LCDR1 is set forth as LX1, LX2, G S K LX3 V (SEQ ID NO: 144), wherein LX1, LX2, and LX3 are any amino acid; wherein the amino acid sequence of LCDR2 is set forth as D D LX4, wherein LX4 is any amino acid; and wherein the amino acid sequence of LCDR3 is set forth as Q V W D LX5 LX6 S D LX7 V V (SEQ ID NO; 146), wherein LX5, LX6, and LX7 are any amino acid; or a combination of (a) and (b).

In a related aspect, wherein the amino acid sequence of HCDR2 is set forth in SEQ ID NO: 137, wherein HX1 is selected from the group consisting of W and S; wherein the amino acid sequence of HCDR3 is set forth in SEQ ID NO: 138, wherein HX2 is selected from the group consisting of A and S, wherein HX3 is P, wherein HX4 is Q, wherein HX5 is W, wherein HX6 is selected from the group consisting of E, Q, M, L, and V, wherein HX7 is selected from the group consisting of L, W, and Y, wherein HX8 is selected from the group consisting of V and T, wherein HX9 is selected from the group consisting of H, A, S, wherein HX10 is E, wherein HX11 is A, wherein HX12 is selected from the group consisting of I, L, and M; wherein the amino acid sequence of LCDR1 is set forth in SEQ ID NO: 139, wherein LX1 is selected from the group consisting of N, L, and I, wherein LX2 is selected from the group consisting of L and I, wherein LX3 is selected from the group consisting of S and L; wherein the amino acid sequence of LCDR2 is set forth as D D LX4, wherein LX4 is selected from the group consisting of S and G; wherein the amino acid sequence of LCDR3 is set forth in SEQ ID NO: 141, wherein LX5 is selected from the group consisting of S and T, wherein LX6 is selected from the group consisting of S and G, and wherein LX7 is selected from the group consisting of H and G.

In a further related aspect of the isolated dual binding antibody, HX1 is W, HX2 is selected from the group consisting of A and S, HX6 is selected from the group consisting of E and M, HX7 is selected from the group consisting of L and W, HX8 is selected from the group consisting of V and T, HX9 is selected from the group consisting of H and A, HX12 is selected from the group I and L, LX1 is L, LX2 is I, LX3 is L, LX4 is selected from the group consisting of S and G, LX5 is S, LX6 is S, and LX7 is selected from the group consisting of H and G.

In yet another related aspect of isolated dual binding antibody, an isolated dual bind antibody comprises CDRs wherein HX1 is W, HX2 is A, HX6 is E, HX7 is L, HX8 is T, HX9 is A, HX12 is I, LX4 is S, and LX7 is G; or HX1 is W, HX2 is A, HX6 is M, HX7 is L, HX8 is V, HX9 is A, HX12 is L, LX4 is S, and LX7 is H; or HX1 is W, HX2 is S, HX6 is E, HX7 is W, HX8 is V, HX9 is H, HX12 is L, LX4 is G, and LX7 is G.

In another related aspect of the isolated dual binding antibody, said VH domain comprising the amino acid sequence set forth in SEQ ID NO: 1 with at least one amino acid variant at any of positions 52, 99, 100, 101, 102, 103, 104, 105, 106, 107, 108, or 111, or any combination thereof (IMGT positions: 57, 107, 108, 109, 110, 111, 111A, 112A, 112, 113, 114, or 117, or a combination thereof); said VL domain comprising the amino acid sequence set forth in SEQ ID NO: 2 with at least one amino acid variant at any of positions 26, 27, 31, 51, 56, 77, 92, 93, or 96, or any combination thereof (IMGT positions: 27, 28, 38, 65, 70, 94, 109, 110, or 115, or a combination thereof); or a combination of the VH domain set forth in (a) and the VL domain set forth in (b); wherein the total number of variant positions in said VH domain, said VL domain, or said combination thereof of said dual binding antibody, is at least 2.

In a further related aspect, said at least one variant amino acid in said VH domain comprises a variant at position 106 of SEQ ID NO: 1 (IMTG position 112). In another further related aspect, the amino acid sequence of said VH domain is selected from the sequences set forth in SEQ ID Nos: 4, 6, 8, 10, 12, 14, 16, 18, 20, 22, 24, 26, 28, 30, 32, 34, 36, 38, 40, 42, 44, 46, 48, 50, 52, and 54. In yet another further related aspect, said at least one amino acid variant in said VL domain comprises a variant amino acid in a CDR region. In still another further related aspect, said variant amino acid in said VL domain comprises a variant at any of positions 26, 27, 31, or 96 of SEQ ID NO: 2, or a combination thereof (IMGT positions: 27, 28, 38, or 115, or a combination thereof). In another further related aspect, there are at least two variants in said VL domain and the second variant comprises a variant amino acid in a framework region. In yet another further related aspect, said variant amino acid in a framework region comprises a variant at position 56 or 77 of SEQ ID NO: 2, or a combination thereof (IMGT positions: 70 or 94, or a combination thereof).

In another related aspect, the amino acid sequence of said VL domain is selected from the sequences set forth in SEQ ID Nos: 3, 5, 7, 9, 11, 13, 15, 17, 19 21, 23, 25, 27, 29, 31, 33, 35, 37, 39, 41, 43, 45, 47, 49, 51, and 53. In still another related aspect, the amino acid sequences of a VH domain-VL domain pair are selected from the pair sequences set forth in SEQ ID Nos: 4 and 3, SEQ ID Nos: 6 and 5, SEQ ID Nos: 8 and 7, SEQ ID Nos: 10 and 9, SEQ ID Nos: 12 and 11, SEQ ID Nos: 14 and 13, SEQ ID Nos: 16 and 15, SEQ ID Nos: 18 and 17, SEQ ID Nos: 20 and 19, SEQ ID Nos: 22 and 21, SEQ ID Nos: 24 and 23, SEQ ID Nos: 26 and 25, SEQ ID Nos: 28 and 27, SEQ ID Nos: 30 and 29, SEQ ID Nos: 32 and 31, SEQ ID Nos: 34 and 33, SEQ ID Nos: 36 and 35, SEQ ID Nos: 38 and 37, SEQ ID Nos: 40 and 39, SEQ ID Nos: 42 and 41, SEQ ID Nos: 44 and 43, SEQ ID Nos: 46 and 45, SEQ ID Nos: 48 and 47, SEQ ID Nos: 50 and 49, SEQ ID Nos: 52 and 51, and SEQ ID Nos: 54 and 53.

In another embodiment, the isolated dual binding antibody disclosed herein comprises a heavy chain variable domain (VH) and a light chain variable domain (VL), wherein the VH and VL comprise the amino acid sequences of SEQ ID Nos:209 and 210.

In another embodiment, the isolated dual binding antibody disclosed herein comprises a heavy chain variable domain (VH) and a light chain variable domain (VL), wherein the VH and VL comprise the amino acid sequences of SEQ ID Nos:219 and 220.

In another embodiment, the isolated dual binding antibody disclosed herein comprises a heavy chain variable domain (VH) and a light chain variable domain (VL), wherein the VH and VL comprise the amino acid sequences of SEQ ID Nos:249 and 250.

In another embodiment, the isolated dual binding antibody disclosed herein comprises a heavy chain variable domain (VH) and a light chain variable domain (VL), wherein the VH and VL comprise the amino acid sequences of SEQ ID Nos:337 and 338.

In another embodiment, the isolated dual binding antibody disclosed herein comprises a heavy chain variable domain (VH) and a light chain variable domain (VL), wherein the VH and VL comprise the amino acid sequences as shown in Table 1 or Table 10.

In another related aspect, the dual binding antibody comprises an IgG, an Fv, an scFv, an Fab, an F(ab′)₂, a minibody, a diabody, or a triabody. In a further related aspect, the IgG comprises IgG1, IgG2, IgG3, or an IgG4. In still another related aspect, said IgG comprises a mutated IgG which is unable to bind to antibody-dependent cellular cytotoxicity components.

Disclosed herein, in one aspect is a composition comprising the isolated dual binding antibody and a pharmaceutically acceptable carrier.

Disclosed herein, in one aspect is a nucleic acid construct, comprising a nucleic acid sequence encoding a dual binding antibody, said antibody comprising an antibody antigen-binding domain site comprising a heavy chain variable region (VH) domain and a light chain variable region (VL) domain, wherein said VH domain comprises a set of CDRs, HCDR1, HCDR2, and HCDR3, wherein the amino acid sequence of HCDR1 is set forth in SEQ ID NO: 136; wherein the amino acid sequence of HCDR2 is set forth as: I HX1 Y D G S N K (SEQ ID NO: 142), wherein HX1 is any amino acid; and wherein the amino acid sequence of HCDR3 is set forth as: A R HX2 HX3 HX4 HX5 HX6 HX7 HX8 HX9 HX10 HX11 F D HX12 (SEQ ID NO: 143), wherein XH2, HX3, HX4, HX5, HX6, HX7, HX8, HX9, HX10, HX11, and HX12 are any amino acid; or wherein said VL domain comprises a set of CDRs, LCDR1, LCDR2, and LCDR3, wherein the amino acid sequence of LCDR1 is set forth as LX1, LX2, G S K LX3 V (SEQ ID NO: 144), wherein LX1, LX2, and LX3 are any amino acid; wherein the amino acid sequence of LCDR2 is set forth as D D LX4, wherein LX4 is any amino acid; and wherein the amino acid sequence of LCDR3 is set forth as Q V W D LX5 LX6 S D LX7 V V (SEQ ID NO; 146), wherein LX5, LX6, and LX7 are any amino acid; or a combination of (a) and (b).

In a related aspect of the nucleic acid, the encoded amino acid sequence of HCDR2 is set forth in SEQ ID NO: 137, wherein HX1 is selected from the group consisting of W and S; wherein the amino acid sequence of HCDR2 is set forth in SEQ ID NO: 138, wherein HX2 is selected from the group consisting of A and S, wherein HX3 is P, wherein HX4 is Q, wherein HX5 is W, wherein HX6 is selected from the group consisting of E, Q, M, L, and V, wherein HX7 is selected from the group consisting of L, W, and Y, wherein HX8 is selected from the group consisting of V and T, wherein HX9 is selected from the group consisting of H, A, S, wherein HX10 is E, wherein HX11 is A, wherein HX12 is selected from the group consisting of I, L, and M; wherein the amino acid sequence of LCDR1 is set forth in SEQ ID NO: 139, wherein LX1 is selected from the group consisting of N, L, and I, wherein LX2 is selected from the group consisting of L and I, wherein LX3 is selected from the group consisting of S and L; wherein the amino acid sequence of LCDR2 is set forth as D D LX4, wherein LX4 is selected from the group consisting of S and G; wherein the amino acid sequence of LCDR3 is set forth in SEQ ID NO: 141, wherein LX5 is selected from the group consisting of S and T, wherein LX6 is selected from the group consisting of S and G, and wherein LX7 is selected from the group consisting of H and G.

In a related aspect of the nucleic acid, the encoded amino acid for HX1 is W, HX2 is selected from the group consisting of A and S, HX6 is selected from the group consisting of E and M, HX7 is selected from the group consisting of L and W, HX8 is selected from the group consisting of V and T, HX9 is selected from the group consisting of H and A, HX12 is selected from the group I and L, LX1 is L, LX2 is I, LX3 is L, LX4 is selected from the group consisting of S and G, LX5 is S, LX6 is S, and LX7 is selected from the group consisting of H and G. A further related aspect, wherein the encoded amino acid for HX1 is W, HX2 is A, HX6 is E, HX7 is L, HX8 is T, HX9 is A, HX12 is I, LX4 is S, and LX7 is G; or HX1 is W, HX2 is A, HX6 is M, HX7 is L, HX8 is V, HX9 is A, HX12 is L, LX4 is S, and LX7 is H; or HX1 is W, HX2 is S, HX6 is E, HX7 is W, HX8 is V, HX9 is H, HX12 is L, LX4 is G, and LX7 is G.

In a related aspect of the nucleic acid construct, said VH domain comprising the amino acid sequence set forth in SEQ ID NO: 1 with at least one amino acid variant at any of positions 52, 99, 100, 101, 102, 103, 104, 105, 106, 107, 108, or 111, or any combination thereof (IMGT positions: 57, 107, 108, 109, 110, 111, 111A, 112A, 112, 113, 114, or 117, or a combination thereof); said VL domain comprising the amino acid sequence set forth in SEQ ID NO: 2 with at least one amino acid variant at any of positions 26, 27, 31, 51, 56, 77, 92, 93, or 96, or any combination thereof (IMGT positions: 27, 28, 38, 65, 70, 94, 109, 110, or 115, or a combination thereof); or a combination of VH domain set forth in (a) and the VL domain set forth in (b); wherein the total number of variant positions in the encoded VH domain, the encoded VL domain, or a combination thereof, is at least 2. In a further related aspect, said sequence comprises two nucleic acid sequences, one encoding the variant dual binding antibody VH domain, and one encoding the variant dual binding antibody VL domain. In a further related aspect, the nucleic acid sequence encoding said VH domain is selected from the sequences set forth in SEQ ID Nos: 57, 59, 61, 63, 65, 67, 69, 71, 73, 75, 77, 79, 81, 83, 85, 87, 89, 91, 93, 95, 97, 99, 101, 105, and 107. In another further related aspect, the nucleic acid sequence encoding said VL domain is selected from the sequences set forth in SEQ ID Nos: 58, 60, 62, 64, 66, 68, 70, 72, 74, 76, 78, 80, 82, 84, 86, 88, 90, 92, 94, 96, 98, 100, 102, 104, 106, and 108. In yet another further related aspect, the nucleic acid sequences encoding the dual antibody VH domain-VL domain pair are selected from the paired sequences set forth in SEQ ID Nos: 57 and 58, SEQ ID Nos: 59 an 60, SEQ ID Nos: 61 and 62, SEQ ID Nos: 63 and 64, SEQ ID Nos: 65 and 66, SEQ ID Nos: 67 and 68, SEQ ID Nos: 69 and 70, SEQ ID Nos: 71 and 72, SEQ ID Nos: 73 and 74, SEQ ID Nos: 75 and 76, SEQ ID Nos: 77 and 78, SEQ ID Nos: 79 and 80, SEQ ID Nos: 81 and 82, SEQ ID Nos: 83 and 84, SEQ ID Nos: 85 and 86, SEQ ID Nos: 87 and 88, SEQ ID Nos: 89 and 90, SEQ ID Nos: 91 and 92, SEQ ID Nos: 93 and 94, SEQ ID Nos: 95 and 96, SEQ ID Nos: 97 and 98, SEQ ID Nos: 99 and 100, SEQ ID Nos: 101 and 102, SEQ ID Nos: 103 and 104, SEQ ID Nos: 105 and 106, and SEQ ID Nos: 107 and 108.

In a related aspect of the nucleic acid construct, said antibody comprises an IgG, a Fv, a scFv, a Fab, a F(ab′)₂, a minibody, a diabody, or a triabody. In a further related aspect, said IgG comprises a mutated IgG which is unable to bind to antibody-dependent cellular cytotoxicity components.

In another related aspect, the nucleic acid construct further comprises a regulatory sequence operably linked to said nucleic acid sequence.

Disclosed herein, in one aspect is an expression vector comprising the nucleic acid construct encoding a dual binding antibody, said antibody comprising an antibody antigen-binding domain site comprising a heavy chain variable region (VH) domain and a light chain variable region (VL) domain.

Disclosed herein, in one aspect is a host cell comprising the expression vector comprising a nucleic acid construct encoding a dual binding antibody, said antibody comprising an antibody antigen-binding domain site comprising a heavy chain variable region (VH) domain and a light chain variable region (VL) domain Disclosed herein, in one aspect is a composition comprising a nucleic acid construct encoding a dual binding antibody, said antibody comprising an antibody antigen-binding domain site comprising a heavy chain variable region (VH) domain and a light chain variable region (VL) domain and a pharmaceutically acceptable carrier.

Disclosed herein, in one aspect is a method of producing a dual binding antibody comprising an antibody antigen-binding domain site comprising a heavy chain variable region (VH) domain and a light chain variable region (VL) domain, said method comprising culturing the host cell comprising the expression vector comprising a nucleic acid construct encoding a dual binding antibody, said antibody comprising an antibody antigen-binding domain site comprising a heavy chain variable region (VH) domain and a light chain variable region (VL) domain; expressing said nucleic acid construct from said vector; isolating said dual binding antibody.

Disclosed herein, in one aspect is a library of immunoglobulins or fragments thereof comprising an antibody antigen-binding domain site comprising a heavy chain variable region (VH) domain and a light chain variable region (VL) domain, wherein said VH domain comprises a set of CDRs, HCDR1, HCDR2, and HCDR3, wherein the amino acid sequence of HCDR1 is set forth in SEQ ID NO: 136; wherein the amino acid sequence of HCDR2 is set forth as: I HX1 Y D G S N K (SEQ ID NO: 142), wherein HX1 is any amino acid; and wherein the amino acid sequence of HCDR3 is set forth as: A R HX2 HX3 HX4 HX5 HX6 HX7 HX8 HX9 HX10 HX11 F D HX12 (SEQ ID NO: 143), wherein XH2, HX3, HX4, HX5, HX6, HX7, HX8, HX9, HX10, HX11, and HX12 are any amino acid; or wherein said VL domain comprises a set of CDRs, LCDR1, LCDR2, and LCDR3, wherein the amino acid sequence of LCDR1 is set forth as LX1, LX2, G S K LX3 V (SEQ ID NO: 144), wherein LX1, LX2, and LX3 are any amino acid; wherein the amino acid sequence of LCDR2 is set forth as D D LX4, wherein LX4 is any amino acid; and wherein the amino acid sequence of LCDR3 is set forth as Q V W D LX5 LX6 S D LX7 V V (SEQ ID NO; 146), wherein LX5, LX6, and LX7 are any amino acid.

In a related aspect of the library, the amino acid sequence of HCDR2 is set forth in SEQ ID NO: 137, wherein HX1 is selected from the group consisting of W and S; wherein the amino acid sequence of HCDR2 is set forth in SEQ ID NO: 138, wherein HX2 is selected from the group consisting of A and S, wherein HX3 is P, wherein HX4 is Q, wherein HX5 is W, wherein HX6 is selected from the group consisting of E, Q, M, L, and V, wherein HX7 is selected from the group consisting of L, W, and Y, wherein HX8 is selected from the group consisting of V and T, wherein HX9 is selected from the group consisting of H, A, S, wherein HX10 is E, wherein HX11 is A, wherein HX12 is selected from the group consisting of I, L, and M; wherein the amino acid sequence of LCDR1 is set forth in SEQ ID NO: 139, wherein LX1 is selected from the group consisting of N, L, and I, wherein LX2 is selected from the group consisting of L and I, wherein LX3 is selected from the group consisting of S and L; wherein the amino acid sequence of LCDR2 is set forth as D D LX4, wherein LX4 is selected from the group consisting of S and G; wherein the amino acid sequence of LCDR3 is set forth in SEQ ID NO: 141, wherein LX5 is selected from the group consisting of S and T, wherein LX6 is selected from the group consisting of S and G, and wherein LX7 is selected from the group consisting of H and G.

In a further related aspect of the library, HX1 is W, HX2 is selected from the group consisting of A and S, HX6 is selected from the group consisting of E and M, HX7 is selected from the group consisting of L and W, HX8 is selected from the group consisting of V and T, HX9 is selected from the group consisting of H and A, HX12 is selected from the group I and L, LX1 is L, LX2 is I, LX3 is L, LX4 is selected from the group consisting of S and G, LX5 is S, LX6 is S, and LX7 is selected from the group consisting of H and G.

In yet another further related aspect of the library, HX1 is W, HX2 is A, HX6 is E, HX7 is L, HX8 is T, HX9 is A, HX12 is I, LX4 is S, and LX7 is G; HX1 is W, HX2 is A, HX6 is M, HX7 is L, HX8 is V, HX9 is A, HX12 is L, LX4 is S, and LX7 is H; or HX1 is W, HX2 is S, HX6 is E, HX7 is W, HX8 is V, HX9 is H, HX12 is L, LX4 is G, and LX7 is G. In still another further related aspect of the library, said VH domain comprising the amino acid sequence set forth in SEQ ID NO: 1 with at least one amino acid variant at any of positions 52, 99, 100, 101, 102, 103, 104, 105, 106, 107, 108, or 111, or any combination thereof (IMGT positions: 57, 107, 108, 109, 110, 111, 111A, 112A, 112, 113, 114, or 117, or a combination thereof); said VL domain comprising the amino acid sequence set forth in SEQ ID NO: 2 with at least one amino acid variant at any of positions 26, 27, 31, 51, 56, 77, 92, 93, or 96, or any combination thereof (IMGT positions: 27, 28, 38, 65, 70, 94, 109, 110, or 115, or a combination thereof); or a combination of the VH domain set forth in (a) and the VL domain set forth in (b); wherein the total number of variant positions in the VH domain, the VL domain, or a combination thereof is at least 2.

In another related aspect of the library, the immunoglobulin comprises an IgG, an Fv, an scFv, an Fab, an F(ab′)₂, a minibody, a diabody, or a triabody.

In another related aspect of the library, the IgG comprises a mutated IgG which is unable to bind to antibody-dependent cellular cytotoxicity components.

Disclosed herein in one aspect, is a method of treating a subject suffering from a disease or condition comprising an allergic or respiratory condition, an inflammatory and/or autoimmune condition of the skin or gastrointestinal organs; scleroderma; or tumors or cancers including Hodgkin's lymphoma, said method comprising administering to said subject an isolated dual binding antibody disclosed herein.

In one related aspect of the method of treating a subject, said allergic or respiratory condition is asthma; allergic asthma; nonallergic asthma; severe asthma; mild asthma; chronic obstructive pulmonary disease (COPD); a condition involving airway inflammation including eosinophilia, fibrosis and excess mucus production, cystic fibrosis, allergic lung disease, airway hyperresponsiveness, goblet cell metaplasia, mucus hypersecretion, airway remodeling, pulmonary fibrosis; atopic disorders including atopic dermatitis, urticaria, eczema, allergic enterogastritis, and allergic rhinitis; or a combination thereof; or said inflammatory and/or autoimmune conditions including inflammatory bowel diseases (IBD) and liver conditions including cirrhosis or fibrosis; or a combination thereof.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The subject matter of engineered dual binding antibodies is particularly pointed out and distinctly claimed in the concluding portion of the specification. These dual binding antibodies, however, both as to their generation and method of use, together with objects, features, and advantages thereof, may best be understood by reference to the following detailed description when read with the accompanying drawings in which:

FIGS. 1A and 1B present the template antibody heavy chain (SEQ ID NO: 1) (FIG. 1A) and light chain (SEQ ID NO: 2) amino acid sequences (FIG. 1B), respectively, indicating the framework (FR) and complementarity-determining regions (CDR) regions. For the Heavy (H) chain, the different regions are labeled FR1, CDR1, FR2, CDR2, FR3, CDR3, and FR4, and in some embodiments are referred to as HFR1, HCDR1, HFR2, HCDR2, HFR3, HCDR3, and HFR4. For the Light (L) chain, the different regions are labeled FR1, CDR1, FR2, CDR2, FR3, CDR3, and FR4, and in some embodiments are referred to as LFR1, LCDR1, LFR2, LCDR2, LFR3, LCDR3, and LFR4. Below the template amino acid sequences, the variant amino acids of the engineered dual binding clones are displayed and aligned within the CDR and FR regions.

FIGS. 2A and 2B present bar graphs showing binding of re-epitoped antibodies displayed on yeast to recombinant human IL-13 (rh-IL-13) (FIG. 2A) or recombinant human TSLP (rhTSLP) (FIG. 2B). FIG. 2A shows binding of isolated yeast-surface display anti-IL13 clones to 10 nM rh-IL-13. FIG. 2B shows binding of isolated yeast-surface display anti-TSLP clones to 10 nM rhTSLP. Data was normalized to the yeast surface expression levels of each clone, and to an anti-hIL-13 and anti-hTSLP mean fluorescence intensity (MFI) binding signal of positive control yeast clones.

FIGS. 3A-3F presents size exclusion chromatography (SEC) scans of a human standard IgG1 (FIG. 3A), BDG33.003 (FIG. 3B), BDG33.004 (FIG. 3C), BDG 33.005 (FIG. 3D), BDG33.023 (FIG. 3E), and BDG33.025 (FIG. 3F). The purified IgGs were run on a GE Superdex®200 10/300 increase (column volume (CV)=25 ml) in PBS buffer at 0.5 ml/min. In the antibody scans shown in FIGS. 3B-3D, the leading peak corresponds to (0.36 CV) that typical of a large aggregate, and a second peak with retention of approximately 13.2 ml (0.528 CV) that is typical of an ordinary human IgG. Area Under the Curve (AUC) peak ratio is approximately 23% misfolded/77% folded IgG fraction, respectively. For the antibody scans shown in FIGS. 3E-3F, the leading peak corresponds to (0.36 CV) that typical of a large diameter aggregate, and a second peak with retention of approximately 13.8 ml (0.55 CV) that is typical of an ordinary human IgG. Area Under the Curve (AUC) peak ratio is 97.3% folded/2.8% misfolded and 98.5% folded/1.5% misfolded for BDG33.023 (FIG. 3E) and BDG33.025 (FIG. 3F) respectively.

FIGS. 4A and 4B present Differential Scanning Fluorimetry (DSF) analysis of the melting point of indicated IgGs BDG33.023 (FIG. 4A) and BDG33.025 (FIG. 4B). Light gray dashed line in the upper graph represents the T-onset and bold gray dashed lines represents the Tm1 and Tm2. The lower graph is the 1st derivative of the measurement. FIG. 4A: DSF of BDG33.023 T-onset of 64.2° C. and first transition point at 67.7° C. FIG. 4B: DSF of BDG33.025 T-onset of 56.4° C., first transition point at 60.9° C. and second transition point at 67.4° C.

FIGS. 5A-5F presents Surface Plasmon Resonance (SPR) analysis of antibodies binding to human IL-13, Cyno IL-13, and human TSLP. Representative SPR sonograms of BDG33.003 and BDG33.004 binding to IL-13 are presented in FIGS. 5A-5D. Recombinant human IL-13 (rh-IL-13) was tested at 800 nM with a 2-fold dilution (FIGS. 5A-5B). Recombinant cyno IL-13 (rc-IL-13) was tested at 200 nM with a 2-fold dilution (FIGS. 5C-5D). Representative SPR sensorgrams of BDG33.003 and BDG33.004 binding to human TSLP (h-TSLP) are presented in FIGS. 5E and 5F. hTSLP served as analyte at concentrations of 3.2 nM to 0.2 nM with two-fold dilutions (FIGS. 5E-F). Representative SPR sensorgrams of BDG33.023 and BDG33.025 binding to human IL-13 (h-IL-13) are presented in FIGS. 5G and 5H. hIL-13 served as analyte at concentrations of 20 nM to 0.6 nM with tow fold dilutions (FIGS. 5G-5H).

FIGS. 6A-6E present ELISA EC50 binding of BDG33.023 and BDG33.025 to human TSLP, cytomegaly monkey (cyno) TSLP or cytomegaly monkey (cyno) IL-13. Binding of BDG33.023 (filled circles) and BDG33.025 (filled squares) to human TSLP (FIG. 6A-human TSLP). Binding of BDG33.023 to cyno-TSLP (FIG. 6B-33.023 cyno TSLP). Binding of BDG33.025 to cyno-TSLP (FIG. 6C-33.025 cyno TSLP). Binding of BDG33.023 to cyno-IL-13 (FIG. 6D-33.023 cyno IL-13). Binding of BDG33.025 to cyno-IL-13 (FIG. 6E-33.025 cyno IL-13).

FIGS. 7A-7D present competitive binding assay of antibodies to hTSLP or hIL-13. FIG. 7A: Indicated antibodies (anti-TSLP-control; anti-IL-13 control; BDG33.023; BDG33.025) were pre-incubated with increasing levels of hIL-13 and added to a plate that was pre-coated with hIL-13. BDG33.023 and BDG33.025 binding to plate-bound hIL-13 was inhibited as soluble hIL-13 concentration increased. FIG. 7B: Indicated antibodies (anti-TSLP-control; anti-IL-13 control; BDG33.023; BDG33.025) were pre-incubated with increasing levels of hTSLP and added to a plate that was pre-coated with hTSLP. BDG33.023 and BDG33.025 binding to plate-bound hTSLP was inhibited as soluble hTSLP concentration increased. FIG. 7C: Antibodies (anti-TSLP-control; anti-IL-13 control; BDG33.023; BDG33.025) were pre-incubated with increasing levels of hTSLP and added to a plate pre-coated with hIL-13, BDG33.023 binding to IL-13 was inhibited as soluble hTSLP concentration increased. FIG. 7D: Indicated antibodies (anti-TSLP-control; anti-IL-13 control; BDG33.023; BDG33.025) were pre-incubated with increasing levels of hIL-13 and added to a plate pre-coated with hTSLP. BDG33.023 binding to plate-bound hTSLP was inhibited as soluble hIL-13 concentration increased. Anti-TSLP and anti-IL-13 control antibodies only showed binding with their respective ligands, and only competed with their respective ligands.

FIG. 8 presents the results of an ELISA specificity test that compared non-specific binding to specific binding of BDG330.23 and BDG33.025. The ELISA plate was coated with hIL-13, hTSLP, and the non-related cytokines IL-2, IL-17, and IL-4. BSA binding signal corresponds to assay background level.

FIG. 9 presents the results of an IC50 inhibition assay that measured IgG specific blocking of hTSLP from binding to an ELISA plate coated with TSLP receptor (TSLP-R). The X-axis represents concentration of competitor. Competitors: TSLP-R (black circles) with a resultant IC50=3 nM; and BGD33.023 (black triangles) with a resultant IC50=0.41 nM.

FIG. 10 presents a schematic representation of the HEK-Blue IL-13 system downstream signaling.

FIGS. 11A-11D present hIL-13 pSTAT6 signaling inhibition data. The results are based on stimulation of HEK-Blue cell's IL-13 activation pathway by recombinant rh-IL-13 and inhibition of this stimulation by indicated IgGs. HEK-Blue IL-13 cells (50,000 cells/well) were incubated with rh-IL-13 at a range of concentrations (0 nM-8 nM). IL-13 downstream signaling was quantified with QUANTI-Blue 24 h post incubation (FIG. 11A). hIL-13 downstream inhibition on HEK-BLUE IL-13 cells by engineered dual binding antibodies was analyzed as follows. rh-IL-13 (0.4 nM) was incubated with indicated antibodies at an antibody concentration range of 0 nM-750 nM. Antibodies assayed were BDG33.002 (positive control), BDG33.003 (Clone C2), and BDG33.006 (negative control), respectively (FIG. 11B). Clones BDG33.023 and BDG33.025 were assayed at an antibody concentration range of 0 nM-100 nM (FIGS. 11C and 11D show 33.023 IL-13 pSTAT6 inhibition, and 33.025 IL-13 pSTAT6 inhibition, respectively. After the incubation the hIL-13/IgG mixture was added to the cells, secreted embryonic alkaline phosphatase (SEAP) activity was quantified with QUANTI-Blue 24 h post incubation. Data shown is the mean of triplicate experiments, and error bars represent standard deviation.

FIGS. 12A-12C present TSLP signaling pathway inhibition data TSLP dependent pSTAT5 signaling activation pathway, and inhibition of the activation by BDG 33.023 in human leukemia MUTZ5 cells. FIG. 12A shows flow-cytometry analysis of MUTZ 5 CD127 (IL-7a) receptor and TSLP-R receptor expression, as follows. Unstained cells (panel a), cells stained for CD127+ wherein approximately 36% of the total cell population was labeled (panel b), cells stained for TSLP-R+, wherein approximately 96% of the total cell population was labeled (panel c), and cells stained for both TSLP-R+ and CD127+ wherein approximately 41% of total cell population was labeled (panel d) (FIG. 12A). FIG. 12B shows MUTZ5 pSTAT5 activation. EC50 of hTSLP phosphor-STATS (pSTAT5) activation in MUTZ5 cells. Percent (%) positive cells represents pSTAT5 positive cells as a percentage of the parent population. FIG. 12C shows inhibition of MUTZ5 pSTAT5 activation. IC50 of BDG33.023 inhibition of TSLP dependent pSTAT5 activation in MUTZ5 cells. TSLP was pre-incubated for 30 min with 0.48 pM to 500 pM of BDG33.023 and added to MUTZ5 cells. Positive cells are representing pSTAT5 positive population as a percentage of the parent population (FIG. 12C).

FIG. 13 shows retention time and calculated pI for some of the dual binding antibodies disclosed herein. IgG marker retention time was 4.77 min.

FIGS. 14A-14C shows competitive ELISA of some of the dual binding antibodies and 33.001 (Tezepelumab) over TSLP. ELISA plates were coated over night at 4° C. with 50 ng/well of 33.001. Dual binding antibodies were double diluted and pre-incubated with 7 nM constant concentration of TSLP-HIS for 1 hour at room temperature. After blocking and washing steps, the dual binding antibodies-TSLP mix were subjected over the plates, incubated for 10 minutes and washed again before 30 minutes incubation with anti-HIS. The results show all tested dual binding antibodies presented a similar IC50.

FIG. 15 shows results of nanoscale differential scanning fluorimetry (nanoDSF) analysis of some of the dual binding antibodies disclosed herein. Tm threshold for lambda chain was >65° C. and T-onset >60° C.

FIGS. 16A-16F show the results of SPR (Surface Plasmon Resonance) analysis for some of the dual binding antibodies disclosed herein for human IL-13 and TSLP.

FIGS. 17A and B show size exclusion chromatography (SEC) scans (FIG. 17A), and nano-differential scanning fluorimetry (DSF) analysis of the melting point (FIG. 17B) for antibody BDG38.074. Representative analysis of the melting point of indicated IgGs were analyzed in duplicate. Light gray dashed line represents the T-onset and bold gray dashed lines represents the Tm1 and Tm2. FIG. 17B shows the 1st derivative of the measurement. DSF values are summarized in FIG. 15.

FIGS. 18A-18D show binding affinities of representative clone BDG38.74. FIG. 18A shows binding affinities of antibody BDG38.074 to IL-13. The results show antibody BDG38.074 binds human IL-13 with double digit picomolar affinities. FIG. 18B shows binding affinities of antibody BDG38.074 to human TSLP. The results show antibody BDG38.074 binds human TSLP with double digit picomolar affinities. FIG. 18C shows binding affinities of antibody BDG38.074 to cyno IL-13. FIG. 18D shows binding affinities of antibody BDG38.074 to cyno TSLP.

FIGS. 19A and 19B shows size exclusion chromatography (SEC) scans (FIG. 19A), and nano-differential scanning fluorimetry (DSF) analysis of the melting point (FIG. 19B) for antibody BDG38.079. Shown representative DSF analysis of the melting point of indicated IgGs (analyzed in duplicates). Light gray dashed line represents the T-onset and bold gray dashed lines represents the Tm1 and Tm2. FIG. 19B is a graph of the 1st derivative of the measurement. DSF values are summarized in FIG. 15.

FIGS. 20A-20D show IL-13 and TSLP binding of representative clone BDG38.079. FIG. 20A shows binding affinities of antibody BDG38.079 to human IL-13. The results show antibody BDG38.079 binds human IL-13 with double digit picomolar affinities. FIG. 20B shows binding affinities of antibody BDG38.079 to human TSLP. The results show antibody BDG38.079 binds human TSLP with single digit picomolar affinities. FIG. 20C shows binding affinities of antibody BDG38.079 to cyno IL-13. FIG. 20D shows binding affinities of antibody BDG38.079 to cyno TSLP.

FIGS. 21A and 21B show SPR (Surface Plasmon Resonance) analysis of antibodies BDG38.074 and BDG38.079 for human or cyno IL-13 or TSLP.

FIG. 22 shows antibodies BDG38.074 and BDG38.079 inhibit IL-13 function in HEK reporter cell line with double digit picomolar affinity. hIL-13 pSTAT6 signaling inhibition data. The results are based on stimulation of HEK-Blue cell's IL-13 activation pathway by recombinant rh-IL-13 and inhibition of this stimulation by indicated IgGs. rh-IL-13 (0.4 nM) was incubated with indicated antibodies at an antibody concentration range of 0 nM-100 nM After the incubation the hIL-13/IgG mixture was added to the cells, secreted embryonic alkaline phosphatase (SEAP) activity was quantified with QUANTI-Blue 24 h post incubation. Data shown is the mean of triplicate experiments, and error bars represent standard deviation. Antibodies assayed were Tralokinumab, BDG38.074 and BDG38.079 respectively.

FIG. 23 shows antibodies BDG38.074 and BDG38.079 exhibit similar functional inhibition to anti-TSLP benchmarks in MUTZ-5 cell line. MUTZ5 cells were stimulated with human TSLP (hTSLP) and phosphorylated STATS (pSTAT5) staining was evaluated by phospho-flow cytometry.

FIGS. 24A and 24B show inhibition results for representative clones FIG. 24A shows antibodies BDG38.074 and BDG38.079 exhibit similar inhibition of CD23 expression to the anti-IL-13 benchmark (Tralokinumab). IC₅₀ of antibody inhibition of IL-13 was determined by measuring CD23 expression level in monocytes. At the end of 48 hours incubation of the cells with different concentrations of antibodies, monocytes were detached from the bottom of the wells and stained with CD3 (Bio Legend, CAT: 300450), CD14 (Bio Legend, CAT: 301814), CD19 (Bio Legend, CAT: 302212) and CD23 (Bio Legend, CAT: 338506) antibodies. CD23 percentage of CD14+ population was measured using CytoFLEX flow cytometer (Beckman Coulter). FIG. 24B shows antibodies BDG38.074 and BDG38.079 inhibit Thymus and activation-regulated chemokine (TARC) expression similarity to anti-TSLP benchmark (Tezepelumab). IC₅₀ of antibody inhibition of hTSLP was determined by TARC inhibition. TARC levels were determined using TARC DUOSET ELISA kit DY364 (R&D systems) according to kit instructions. ELISA plates were read at 450 nm. Values were analyzed using standard sample curve.

FIGS. 25A and 25B show size exclusion chromatography (SEC) scans (FIG. 25A), and nano-differential scanning fluorimetry (DSF) analysis of the melting point (FIG. 25B) for antibody BDG38.094. Shown representative DSF analysis of the melting point of indicated IgGs (analyzed in duplicates). Light gray dashed line in the upper graph represents the T-onset and bold gray dashed lines represents the Tm1 and Tm2. The FIG. 25B is the 1st derivative of the measurement. DSF values are summarized in Table 11

FIGS. 26A-26D show binding affinities of a representative clone. FIG. 26A shows binding affinities of antibody BDG38.094 to human IL-13. FIG. 26B shows binding affinities of antibody BDG38.094 to human TSLP. FIG. 26C shows binding affinities of antibody BDG38.094 to cyno IL-13. FIG. 26B shows binding affinities of antibody BDG38.094 to cyno TSLP.

FIGS. 27A and 27B show size exclusion chromatography (SEC) scans (FIG. 27A), and nano-differential scanning fluorimetry (DSF) analysis of the melting point (FIG. 27B) for antibody BDG38.138. Shown representative DSF analysis of the melting point of indicated IgGs (analyzed in duplicates). Light gray dashed line in the upper graph represents the T-onset and bold gray dashed lines represents the Tm1 and Tm2. FIG. 27B is the 1st derivative of the measurement. DSF values are summarized in Table 11.

FIGS. 28A-28D show binding affinities of a representative clone. FIG. 28A shows binding affinities of antibody BDG38.138 to human IL-13. FIG. 28B shows binding affinities of antibody BDG38.138 to human TSLP. FIG. 28C shows binding affinities of antibody BDG38.138 to cyno IL-13. FIG. 28D shows binding affinities of antibody BDG38.138 to cyno TSLP.

FIGS. 29A-29D show the results of analyzing BDG38.138 and BDG38.145, which each have different Fc formats. The SPR results demonstrate that antibody clones BDG38.138 and BDG38.145 have similar affinities for human IL-13 and human TSLP. (FIG. 29A) Binding of 38.138 (IgG1 LALAPG) to hIL-13. (FIG. 29B) Binding of 38.145 (IgG1 LALA) to hIL-13. (FIG. 29C) Binding of 38.138 (IgG1 LALAPG) to hTSLP. (FIG. 29D) Binding of 38.145 (IgG1 LALA) to hTSLP.

FIGS. 30A-30C demonstrate that clones BDG38.138 and BDG38.145 with different Fc formats, show similar stability under viral inactivation conditions. (FIG. 30A) SEC analysis of 38.138 and 38.145 after incubation under viral inactivation conditions (sodium acetate (NaAc)) compared to non-treated antibodies (ctrl) and Abs that went through the same viral inactivation process but in PBS (PBS). (FIG. 30B) SDS-PAGE analysis in reduced and a non-reduced conditions of 38.138 and 38.145 after incubation in viral inactivation conditions (NaAc) compared to non-treated antibodies (ctrl) and Ab went the same viral inactivation process but in PBS (PBS pH7.4). (FIG. 30C) HIC analysis of 38.138 and 38.145 after incubation in viral inactivation conditions (NaAc) compared to non-treated antibodies (ctrl) and Abs went the same viral inactivation process but in PBS (PBS). Veltuzumab, Tralokinumab (hIgG1), Tezepelumab (hIgG1) and Dupilumab were analyzed as controls.

FIGS. 31A-31D show that BDG38.138 and BDG38.145, having identical VH/VL sequences but different Fc formats show similar binding under viral inactivation conditions. Binding of 38.138 (hIgG1 LALAPG) and 38.145 (hIgG1 LALA), treated and non-treated in viral inactivation conditions, to human TSLP and human IL-13 was measured by ELISA EC50. (FIG. 31A) ELISA EC50 binding to human TSLP of 38.138 (hIgG1 LALAPG) after incubation in viral inactivation conditions (NaAc) compared to non-treated antibodies (PBS control (ctrl)). Both treated and control antibodies went through the same viral inactivation process but control incubations were in PBS (PBS). Anti-TSLP antibody 33.001 was used as positive control. (FIG. 31B) ELISA EC50 binding to human TSLP of 38.145 (hIgG1 LALA) after incubation in viral inactivation conditions (NaAc) compared to non-treated antibodies (PBS; ctrl). Control antibodies went through the same viral inactivation process but incubations were in PBS (PBS). Anti-TSLP antibody 33.001 was used as a positive control. (FIG. 31C) ELISA EC50 binding to human IL-13 of 38.138 (hIgG1 LALAPG) after incubation in viral inactivation conditions (NaAc) compared to non-treated antibodies (PBS; ctrl). Control antibodies went through the same viral inactivation process but incubation was in PBS (PBS). Anti IL-13 antibody 33.002 was used as a positive control. (FIG. 31D) ELISA EC50 binding to human IL-13 of 38.145 (hIgG1 LALA) after incubation in viral inactivation conditions (NaAc) compared to non-treated antibodies (PBS; ctrl). Control antibodies went through the same viral inactivation process, but incubations were in PBS (PBS). Anti IL-13 antibody 33.002 was used as a positive control.

FIGS. 32A and 32B show comparison of 38.138 and 38.145 antibodies having identical VH/VL sequences but with different Fc formats, incubated with hPBMCs. hPBMCs were stimulated with 0.7 nm hIL-13 and 70 pM hTSLP Immediately following stimulation, cells were treated with antibodies: 38.138 (hIgG1 LALAPG) and 38.145 (hIgG1 LALA), and their respective isotype controls (IC) in concentrations ranging from 0.03 nM to 20 nM. Following 48 hours incubations, cells were analyzed for CD23 expression by flow cytometry, and TARC levels in the supernatant were measured by ELISA. FIG. 32A: CD23 expression levels in monocytes, presented as percentage of the parental population (CD14+ cells). The cells were labeled with fluorescent antibodies to CD14 and CD23. The values shown in the graph are the % of CD23+ cells, out of the CD14+ cells (and not out of the total population). FIG. 32B: TARC levels secreted to the supernatant measured by ELISA.

FIGS. 33A-33B show CD23 expression in monocytes is inhibited by antibody clone BDG38.145. hPBMCs were stimulated for 48 h with 0.7 nm IL-13. FIG. 33A: Gating strategy for CD23+ monocyte population. FIG. 33B: CD23 expression-gating strategy. CD23 expression levels in monocytes, present as percentage of the parental population (CD14+ cells). Tezepelumab, an anti-TSLP antibody, has no effect on CD23 expression. 38.145, lebrikizumab, and tralokinumab lower CD23 expression in a dose-dependent manner 38.145 demonstrates better inhibition properties than tralokinumab and similar inhibition to lebrikizumab.

FIGS. 34A-34C show TARC levels in hPBMCs supernatant is inhibited by BDG38.145. hPBMCs were stimulated for 48 h with either IL-13, TSLP, or with the combination of both cytokines. Immediately following stimulation, cells were treated with tested antibodies: 38.145 compared to Tezepelumab (anti-TSLP), Tralokinumab (anti-IL-13), and Lebrikizumab (anti-IL-13). TARC levels in the supernatant were measured by ELISA. (FIG. 34A) Cells stimulated with 0.7 nM IL-13. TARC levels are inhibited fully by 38.145 and anti-IL-13 Abs. (FIG. 34B) Cells stimulated with 70 pM TSLP. TARC levels are inhibited fully by 38.145 and anti-TSLP Ab. (FIG. 34C) Cells were stimulated with 0.7 nM IL-13 and 70 pM TSLP. In the presence of both cytokines, 38.145 demonstrates full inhibition of TARC while benchmark antibodies show partial inhibitions.

### DETAILED DESCRIPTION

In the following detailed description, numerous specific details are set forth in order to provide a thorough understanding of the engineered dual binding antibodies disclosed herein, including a description of their heavy chain and light chain variable regions. However, it will be understood by those skilled in the art that preparation and use dual binding antibodies may in certain cases, be practiced without these specific details. In other instances, well-known methods, procedures, and components have not been described in detail so as not to obscure the disclosure presented herein.

Antigen binding sequences are conventionally located within the heavy chain and light chain variable region sequences of an antibody. These heavy and light chain variable regions may, in certain instances, be manipulated to create new binding sites, for example to create antibodies or fragments thereof, that bind to a different antigen or an epitope of a different antigen thereof. In some embodiments, as described herein, manipulating the sequence of a heavy chain variable region or the sequence of a light chain variable region, or both, creates a new binding site for an epitope while maintaining antibody functionality. In one embodiment, twenty-one specific sites within the heavy and light chain variable regions are identified, wherein the presence of variant amino acids at these sites, in certain embodiments, creates an engineered dual binding antibody or fragment thereof. In some embodiments, the 21 potential variant sites provide a unique platform from which to engineer dual binding antibodies or fragments thereof.

Disclosed herein are engineered dual binding antibodies or fragments thereof, wherein either a heavy chain variable region, or a light chain variable region, or both, have been mutated to include variant amino acids. In some embodiments, these engineered dual binding antibodies may be identified and selected from a library created to include variant amino acid residues at particular sites within a variable heavy chain or variable light region, or both. In some embodiments, these engineered dual binding antibodies may be produced by specifically mutating target amino acid sites within a variable heavy chain or variable light region, or both. In some embodiments, these engineered dual binding antibodies may be used in a therapeutic method for treating a subject suffering from an allergic or respiratory condition.

### Engineered Dual Binding Antibodies

As used herein, the term “dual binding antibodies” refers to antibodies that have two binding specificities. In certain embodiments, the dual binding antibodies disclosed herein bind to IL-13 and TSLP.

In some embodiments, the present disclosure provides an isolated dual binding antibody comprising three complementarity determining regions (CDRs) on a heavy chain (HCDR1, HCDR2, and HCDR3) and three CDRs on a light chain (LCDR1, LCDR2, and LCDR3) (see e.g. Tables 8 and 9). In some embodiments, the CDRs have the sequences of SEQ ID NOs:149-154. In some embodiments, the dual binding antibody comprises a heavy chain variable domain (VH) and a light chain variable domain (VL) having the amino acid sequences of SEQ ID Nos:155 and 156, or SEQ ID Nos:157 and 158.

In one embodiment, the HCDR1, HCDR2 and HCDR3 comprise the amino acid sequence of SEQ ID NOs:349, 350 and 351 respectively, and the LCDR1, LCDR2 and LCDR3 comprise the amino acid sequence of SEQ ID NO: 359, D D V, and SEQ ID NO: 361, respectively.

In another embodiment, the HCDR1, HCDR2 and HCDR3 comprise the amino acid sequence of SEQ ID NOs:349, 356 and 351 respectively, and the LCDR1, LCDR2 and LCDR3 comprise the amino acid sequence of SEQ ID NO: 364, D D V, and SEQ ID NO: 371, respectively.

In another embodiment, the HCDR1, HCDR2 and HCDR3 comprise the amino acid sequence of SEQ ID NOs:349, 350 and 351 respectively, and the LCDR1, LCDR2 and LCDR3 comprise the amino acid sequence of SEQ ID NO: 362, D D V, and SEQ ID NO: 384, respectively.

In another embodiment, the HCDR1, HCDR2 and HCDR3 comprise the amino acid sequence of SEQ ID NOs:349, 350 and 351 respectively, and the LCDR1, LCDR2 and LCDR3 comprise the amino acid sequence of SEQ ID NO: 364, D D V, and SEQ ID NO: 384, respectively.

In another embodiment, the HCDR1, HCDR2 and HCDR3 comprise the amino acid sequences as shown in Table 8 or Table 4, wherein the LCDR1, LCDR2 and LCDR3 comprise the amino acid sequences as shown in Table 9 or Table 5, respectively.

In some embodiments, disclosed herein is an isolated dual binding antibody comprising three complementarity determining regions (CDRs) on a heavy chain (HCDR1, HCDR2, and HCDR3) and three CDRs on a light chain (LCDR1, LCDR2, and LCDR3), wherein
(i) the HCDR1 comprises the amino acid sequence of SEQ ID NO: 349 or 355, or the amino acid sequence of SEQ ID NO: 149 or SEQ ID NO: 136;
(ii) the HCDR2 comprises the amino acid sequence of one of SEQ ID NOs:350, 352, 354 and 356, or the amino acid sequence of SEQ ID NO: 150 or the sequence set forth as: I HX1 Y D G S N K (SEQ ID NO: 142), wherein HX1 is any amino acid;
(iii) the HCDR3 comprises the amino acid sequence of one of SEQ ID NOs:351, 353, 357, and 358, or the amino acid sequence of SEQ ID NO: 151 or the sequence set forth as: A R HX2 HX3 HX4 HX5 HX6 HX7 HX8 HX9 HX10 HX11 F D HX12 (SEQ ID NO: 143), wherein HX2, HX3, HX4, HX5, HX6, HX7, HX8, HX9, HX10, HX11, and HX12 are any amino acid;
(iv) the LCDR1 comprises the amino acid sequence of one of SEQ ID NOs:359, 362, 364, 366, 369, and 375, or the amino acid sequence of SEQ ID NO: 152 or the sequence set forth as LX1, LX2, G S K LX3 V (SEQ ID NO: 144), wherein LX1, LX2, and LX3 are any amino acid;
(v) the LCDR2 comprises the amino acid sequence of D D V or D D S, or the sequence set forth as D D LX4, wherein LX4 is any amino acid; and
(vi) the LCDR3 comprises the amino acid sequence of one of SEQ ID NOs:361, 363, 365, 368, 370-374, 376-407, or the amino acid sequence of SEQ ID NO: 154 or the sequence set forth as Q V W D LX5 LX6 S D LX7 V V (SEQ ID NO; 146), wherein LX5, LX6, and LX7 are any amino acid.

In some embodiments, disclosed herein is an isolated anti-IL-13, anti-TSLP dual binding antibody comprising a heavy chain variable region (VH) and a light chain variable region (VL), wherein said VH comprises heavy chain complementarity determining regions (HCDRs) HCDR1, HCDR2, and HCDR3, said VL comprises light chain complementarity determining regions (LCDRs) LCDR1, LCDR2, and LCDR3, wherein
(a) the HCDR1 comprises the amino acid sequence set forth in any one of SEQ ID NOs: 349, 355, 149 and 136;
(b) the HCDR2 comprises the amino acid sequence set forth in any one of SEQ ID NOs: 350, 352, 354, 356, and 150 or the sequence set forth as: I HX1 Y D G S N K (SEQ ID NO: 142), wherein HX1 is any amino acid;
(c) the HCDR3 comprises the amino acid sequence set forth in any one of SEQ ID NOs: 351, 353, 357, 358, and 151 or the sequence set forth as: A R HX2 HX3 HX4 HX5 HX6 HX7 HX8 HX9 HX10 HX11 F D HX12 (SEQ ID NO: 143), wherein HX2, HX3, HX4, HX5, HX6, HX7, HX8, HX9, HX10, HX11, and HX12 are any amino acid;
(d) the LCDR1 comprises the amino acid sequence of one of SEQ ID NOs: 364, 359, 362, 366, 369, 375, and 152 or the sequence set forth as LX1, LX2, G S K LX3 V (SEQ ID NO: 144), wherein LX1, LX2, and LX3 are any amino acid;
(e) the LCDR2 comprises the amino acid sequence of DDV or DDS, or the sequence set forth as D D LX4, wherein LX4 is any amino acid; and
(f) the LCDR3 comprises the amino acid sequence set forth in any one of SEQ ID NOs: 384, 361, 363, 365, 368, 370-374, 376-383, 385-407, and 154 or the sequence set forth as Q V W D LX5 LX6 S D LX7 V V (SEQ ID NO; 146), wherein LX5, LX6, and LX7 are any amino acid.

In some embodiments, disclosed herein is an isolated dual binding antibody wherein
the HCDR1, HCDR2 and HCDR3 comprise the amino acid sequences of SEQ ID NOs:349, 350 and 351 respectively, and the LCDR1, LCDR2 and LCDR3 comprise the amino acid sequences of SEQ ID NO: 359, D D V, and SEQ ID NO: 361, respectively; or
the HCDR1, HCDR2 and HCDR3 comprise the amino acid sequences of SEQ ID NOs:349, 356 and 351 respectively, and the LCDR1, LCDR2 and LCDR3 comprise the amino acid sequences of SEQ ID NO: 364, D D V, and SEQ ID NO: 371, respectively; or
the HCDR1, HCDR2 and HCDR3 comprise the amino acid sequences of SEQ ID NOs:349, 350 and 351 respectively, and the LCDR1, LCDR2 and LCDR3 comprise the amino acid sequences of SEQ ID NO: 362, D D V, and SEQ ID NO: 384 respectively; or
the HCDR1, HCDR2 and HCDR3 comprise the amino acid sequences of SEQ ID NOs:349, 350 and 351 respectively, and the LCDR1, LCDR2 and LCDR3 comprise the amino acid sequences of SEQ ID NO: 364, D D V, and SEQ ID NO: 384, respectively.

In some embodiments, disclosed herein is an isolated dual binding antibody, wherein the HCDR1, HCDR2 and HCDR3 comprise the amino acid sequences as shown in Table 8 or Table 4, wherein the LCDR1, LCDR2 and LCDR3 comprise the amino acid sequences as shown in Table 9 or Table 5, respectively.

In some embodiments, disclosed herein is an isolated dual binding antibody, wherein HX1 is W or S; HX2 is A or S; HX3 is P; HX4 is Q; HX5 is W; HX6 is E, Q, M, L, or V; HX7 is L, W, or Y; HX8 is V or T; HX9 is H, A, or S; HX10 is E; HX11 is A; HX12 is I, L, or M; wherein LX1 is N, L, or I; LX2 is L or I; LX3 is S or L; LX4 is S or G; LX5 is S or T; LX6 is S or G; LX7 is H or G.

In some embodiments, disclosed herein is an isolated dual binding antibody, wherein HX1 is W, HX2 is A or S, HX6 is E or M, HX7 is L or W, HX8 is V or T, HX9 is H or A, HX12 is I or L, LX1 is L, LX2 is I, LX3 is L, LX4 is S or G, LX5 is S, LX6 is S, LX7 is H or G.

In some embodiments, disclosed herein is an isolated dual binding antibody, wherein
(a) HX1 is W, HX2 is A, HX6 is E, HX7 is L, HX8 is T, HX9 is A, HX12 is I, LX4 is S, and LX7 is G; or
(b) HX1 is W, HX2 is A, HX6 is M, HX7 is L, HX8 is V, HX9 is A, HX12 is L, LX4 is S, and LX7 is H; or
(c) HX1 is W, HX2 is S, HX6 is E, HX7 is W, HX8 is V, HX9 is H, HX12 is L, LX4 is G, and LX7 is G.

In some embodiments, disclosed herein is an isolated dual binding antibody comprising a heavy chain variable region comprising the amino acid sequence set forth in SEQ ID NO: 1 with at least one amino acid variant at any position or a light chain variable region comprising the amino acid sequence set forth in SEQ ID NO: 2 with at least one amino acid variant at any of position, or a combination thereof, wherein the total number of variant positions in said heavy chain variable region, said light chain variable region, or said combination thereof, is at least 2. In some embodiments, disclosed herein is an isolated dual binding antibody comprising a heavy chain variable region comprising the amino acid sequence set forth in SEQ ID NO: 1 and a light chain variable region comprising the amino acid sequence set forth in SEQ ID NO: 2, wherein there are at least two amino acid variants within the heavy chain variable region or the light chain variable region or the combination thereof. In some embodiments, disclosed herein is an isolated dual binding antibody comprising a heavy chain variable region comprising the amino acid sequence set forth in SEQ ID NO: 1 with at least two amino acid variants at any position and any light chain variable region. In some embodiments, disclosed herein is an isolated dual binding antibody comprising a light chain variable region comprising the amino acid sequence set forth in SEQ ID NO: 2 with at least two amino acid variants at any position and any heavy chain variable region.

As used herein, the term “heavy chain variable region” may be used interchangeable with the term “VH domain” or the term “VH”, having all the same meanings and qualities. As used herein, the term “light chain variable region” may be used interchangeably with the term “VL domain” or the term “VL”, having all the same meanings and qualities.

In certain embodiments, a specific variant VH and/or VL domain, described herein, may be used to screen a library of the complementary variable region to identify VH/VL, respectively, with desirable properties, such as increased affinity for an antigen. Such methods are described, for example, in Portolano et al., J. Immunol. (1993) 150:880-887; Clarkson et al., Nature (1991) 352:624-628. Fischer et al., (2015) Exploiting light chains for the scalable generation and platform purification of native human bispecific IgG. Nature Communications volume 6, Article number: 6113.

Other methods may also be used to mix and match VH and VL domains to identify a Fab or F(ab) 2 having desired dual binding activity. For example: Klimka et al., British Journal of Cancer (2000) 83: 252-260, describe a screening process using a mouse VL and a human VH library with CDR3 and PR4 retained from the mouse VH. After obtaining antibodies, the VH was screened against a human VL library to obtain antibodies that bound antigen. Beiboer et al., J. Mol. Biol. (2000) 296:833-849 describe a screening process using an entire mouse heavy chain and a human light chain library. After obtaining antibodies, one VL was combined with a human VH library with the CDR3 of the mouse retained. Antibodies capable of binding antigen were obtained. Rader et al., PNAS (1998) 95:8910⁻⁸⁹¹⁵ describe a process similar to Beiboer et al above.

These just-described techniques are, in and of themselves, known as such in the art. The skilled person will, however, be able to use such techniques to obtain antigen-binding fragments of antibodies according to several embodiments of the disclosure described herein, using routine methodology in the art.

A skilled artisan would appreciate that dual binding antibody encompasses in its broadest sense an antibody that specifically binds an antigenic determinant of IL-13 and TSLP. The skilled artisan would appreciate that specificity for binding to IL-13 or TSLP reflects that the binding is selective for the antigen and can be discriminated from unwanted or nonspecific interactions. In certain embodiments, the dual binding antibody comprises an antibody fragment or fragments.

In some embodiments, an antigenic determinant comprises an IL-13 or TSLP epitope. The term “epitope” includes any determinant, in certain embodiments, a polypeptide determinant, capable of specific binding to an anti-IL-13 or anti-TSLP binding domain. An epitope is a region of an antigen that is bound by an antibody or an antigen-binding fragment thereof. In some embodiments, the antigen-binding fragment of an antibody comprises a heavy chain variable region, a light chain variable region, or a combination thereof as described herein.

In certain embodiments, epitope determinants include chemically active surface groupings of molecules such as amino acids, sugar side chains, phosphoryl or sulfonyl, and may in certain embodiments have specific three-dimensional structural characteristics, and/or specific charge characteristics. In certain embodiments, the dual binding antibody is said to specifically bind an IL-13 or TSLP epitope when it preferentially recognizes IL-13 or TSLP in a complex mixture of proteins and/or macromolecules. The dual binding antibody is said to specifically bind an epitope when the equilibrium dissociation constant is ≤10⁻⁶, or 10⁻⁷ M. In some embodiments, the equilibrium dissociation constant may be ≤10⁻⁸ M or 10⁻⁹ M. In some further embodiments, the equilibrium dissociation constant may be ≤10⁻¹⁰ M, 10⁻¹¹ M, or 10⁻¹² M. In some embodiments, the equilibrium dissociation constant may be in the range of ≤10⁻⁵ M to 10⁻¹² M.

An antibody binding domain can be a fragment of an antibody or a genetically engineered product of one or more fragments of the antibody, which fragment is involved in specifically binding with the antigen. By “specifically binding” is meant that the binding is selective for the antigen of interest, for example for IL-13 or TSLP in embodiments described herein and can be discriminated from unwanted or nonspecific interactions. As used herein, the term “dual binding antibody” may in certain embodiments, encompass complete immunoglobulin structures, fragments thereof, or domains thereof.

Examples of antibody binding domains include, without limitation, a complementarity determining region (CDR), a variable region (Fv), a VH domain, a light chain variable region (VL), a heavy chain, a light chain, a single chain variable region (scFv), and a Fab fragment. A skilled artisan would appreciate that an scFv is not actually a fragment of an antibody, but instead is a fusion polypeptide comprising the variable heavy chain (VH) and variable light chain (VL) regions of an immunoglobulin, connected by a short linker peptide of for example but not limited to ten to about 25 amino acids. The skilled artisan would also appreciate that the term “Fab” with regard to an antibody, generally encompasses that portion of the antibody consisting of a single light chain (both variable and constant regions) bound to the variable region and first constant region of a single heavy chain by a disulfide bond.

In some embodiments, an antibody encompasses whole antibody molecules, including monoclonal, polyclonal and multispecific (e.g., bispecific) antibodies. In some embodiments, an antibody encompasses an antibody fragment or fragments that retain binding specificity including, but not limited to, variable heavy chain (VH) fragments, variable light chain (VL) fragments, Fab fragments, F(ab′)₂ fragments, scFv fragments, Fv fragments, minibodies, diabodies, triabodies, and tetrabodies (see, e.g., Hudson and Souriau, Nature Med. 9: 129-134 (2003) (hereby incorporated by reference in their entirety)). Also encompassed are humanized, primatized, and chimeric antibodies.

As used herein, in some embodiments, the term “Antibody” may be used interchangeably with the term “Immunoglobulin” having all the same qualities and meanings. Similarly, as used herein, in some embodiments, the term “Antibody or fragments thereof” may be used interchangeably with the term “Immunoglobulin or fragments thereof” having all the same qualities and meanings. Thus, a skilled artisan would appreciate that in some embodiments, “an antibody or fragments thereof”, or “immunoglobulins or fragments thereof” may encompass IgG immunoglobulins or fragments thereof or structures comprising a fragment or fragments thereof, including but not limited to an IgG, an scFv fragment, an Fab fragment, an F(ab′)₂ fragment, Fv fragments, minibodies, diabodies, triabodies, and tetrabodies.

A skilled artisan would recognize that a “Heavy chain variable region” or “VH” with regard to an antibody encompasses the fragment of the heavy chain that contains three CDRs interposed between flanking stretches known as framework (FR) regions, which are more highly conserved than the CDRs, and form a scaffold to support the CDRs. In certain embodiments, the terms a “Heavy chain variable region” or a “VH” may be used interchangeably with “VH domain”.

A skilled artisan would recognize that a “Light chain variable region” or “VL” with regard to an antibody encompasses the fragment of the light chain that contains three CDRs interposed between framework (FR) regions. In certain embodiments, the terms a “Light chain variable region” or a “VL” may be used interchangeably with “VL domain”.

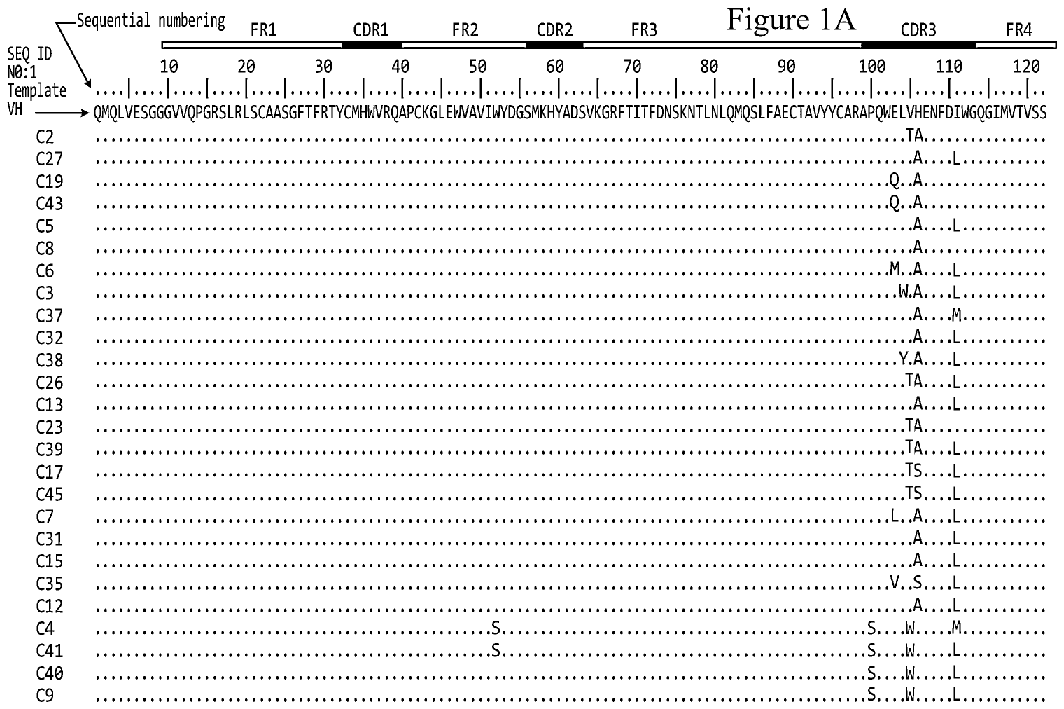

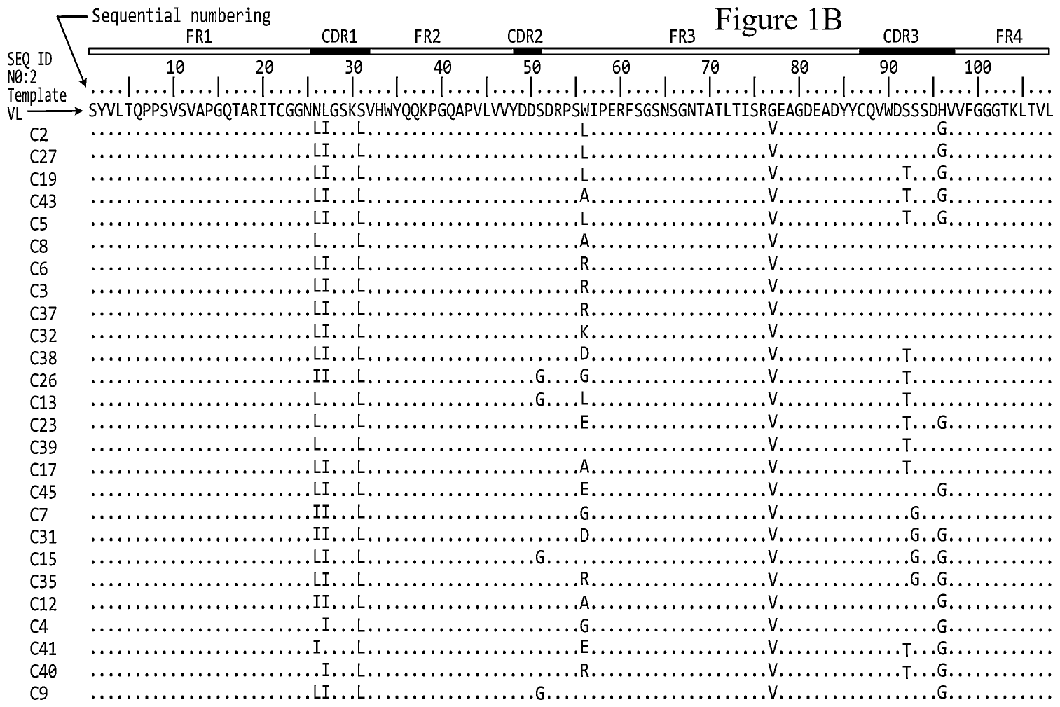

Disclosed herein are a number of amino acid sequences for HCDR1, HCDR2, HCDR3, LCDR1, LCDR2, LCDR3, as well as VH and VL regions for dual binding antibodies that bind to IL-13 and TSLP. Discussion on some embodiments of representative sequences disclosed herein is presented below. FIG. 1A presents the template VH domain amino acid sequence, set forth in SEQ ID NO: 1, and the location of the three heavy-chain (H) CDR regions (HCDR1, HCDR2, HCDR3) and four FR regions (HFR1, HFR2, HFR3, HFR4), while FIG. 1B presents the template VL domain amino acid sequence, set forth in SEQ ID NO: 2, and the location of the three light-chain (L) CDR regions (LCDR1, LCDR2, LCDR3) and four FR regions (LFR1, HCDR2, LFR3, LFR4). The amino acid residues including variant residues, present in each of the CDR regions and each of the FR regions of the re-epitoped clones, are clearly identified by comparing the linear schematic representation of the template VH or template VL sequence with the numbering and amino acids provided below (FIGS. 1A and 1B).

In some embodiments, an isolated dual binding antibody comprises an antibody antigen-binding domain site comprising a VH domain and a VL domain, wherein said VH domain comprises a set of CDRs, HCDR1, HCDR2, and HCDR3, wherein the amino acid sequence of HCDR1 is set forth in SEQ ID NO: 136; wherein the amino acid sequence of HCDR2 is set forth as: I HX1 Y D G S N K (SEQ ID NO: 142), wherein HX1 is any amino acid; and wherein the amino acid sequence of HCDR3 is set forth as: A R HX2 HX3 HX4 HX5 HX6 HX7 HX8 HX9 HX10 HX11 F D HX12 (SEQ ID NO: 143), wherein XH2, HX3, HX4, HX5, HX6, HX7, HX8, HX9, HX10, HX11, and HX12 are any amino acid. A skilled artisan would recognize the 12 unique sites within the VH domain presented in FIG. 1A, wherein a variant amino acid may be found, which are herein identified as HX and may in certain embodiments, encompass the presence of a variant amino acid within the template sequence of the heavy chain.

In some embodiments, a VH domain of a dual binding antibody comprises HCDR1 (SEQ ID NO: 136), HCDR2 (SEQ ID NO: 142), and HCDR3 (SEQ ID NO: 143), wherein the VH domain comprises a variant amino acid at, at least one of HX1, HX2, HX3, HX4, HX5, HX6, HX7, HX8, HX9, HX10, HX11, and HX12.

In some embodiments, a VH domain of a dual binding antibody comprises HCDR1 (SEQ ID NO: 136), HCDR2 (SEQ ID NO: 137) wherein HX1 is selected from the group consisting of W and S; and HCDR3 (SEQ ID NO: 138) wherein HX2 is selected from the group consisting of A and S, wherein HX3 is P, wherein HX4 is Q, wherein HX5 is W, wherein HX6 is selected from the group consisting of E, Q, M, L, and V, wherein HX7 is selected from the group consisting of L, W, and Y, wherein HX8 is selected from the group consisting of V and T, wherein HX9 is selected from the group consisting of H, A, S, wherein HX10 is E, wherein HX11 is A, wherein HX12 is selected from the group consisting of I, L, and M. In certain embodiments, the isolated dual binding antibody comprises variant amino acids comprising CDR1 (SEQ ID NO: 136), CDR2 (SEQ ID NO: 137) wherein HX1 is W, CDR3 (SEQ ID NO: 138) wherein HX2 is selected from the group consisting of A and S, wherein HX3 is P, wherein HX4 is Q, wherein HX5 is W, HX6 is selected from the group consisting of E and M, HX7 is selected from the group consisting of L and W, HX8 is selected from the group consisting of V and T, HX9 is selected from the group consisting of H and A, HX10 is E, HX11 is A, and HX12 is selected from the group I and L.

In some embodiments, the dual binding antibody may have a VH domain comprising an HCDR1 (SEQ ID NO: 136), an HCDR2 (SEQ ID NO: 137) wherein HX1 is W, and an HCDR3 (SEQ ID NO: 138) wherein HX2 is A, HX3 is P, HX4 is Q, HX5 is W, HX6 is E, HX7 is L, HX8 is T, HX9 is A, HX10 is E, HX11 is A, and HX12 is I; or an HCDR1 (SEQ ID NO: 136), an HCDR2 (SEQ ID NO: 137) wherein HX1 is W, and an HCDR3 (SEQ ID NO: 138) wherein HX2 is A, HX3 is P, HX4 is Q, HX5 is W, HX6 is M, HX7 is L, HX8 is V, HX9 is A, HX10 is E, HX11 is A, and HX12 is L; or an HCDR1 (SEQ ID NO: 136), an HCDR2 (SEQ ID NO: 137) wherein HX1 is W, and an HCDR3 (SEQ ID NO: 138) wherein HX2 is S, HX3 is P, HX4 is Q, HX5 is W, HX6 is E, HX7 is W, HX8 is V, HX9 is H, HX10 is E, HX11 is A, and HX12 is L.

Engineered antibody clones having variants in the VH domain as described above, are presented in FIG. 1A.

In some embodiments, an isolated dual binding antibody comprises an antibody antigen-binding domain site comprising a VH domain and a VL domain, wherein said in some embodiments the VL domain comprises a set of CDRs, LCDR1, LCDR2, and LCDR3, wherein the amino acid sequence of LCDR1 is set forth as LX1, LX2, G S K LX3 V (SEQ ID NO: 144), wherein LX1, LX2, and LX3 are any amino acid; wherein the amino acid sequence of LCDR2 is set forth as D D LX4, wherein LX4 is any amino acid; and wherein the amino acid sequence of LCDR3 is set forth as Q V W D LX5 LX6 S D LX7 V V (SEQ ID NO; 146), wherein LX5, LX6, and LX7 are any amino acid. A skilled artisan would recognize the 7 unique sites within the VL domain presented in FIG. 1B, wherein a variant amino acid may be found within a CDR, which are herein identified as LX and may in certain embodiments, encompass the presence of a variant amino acid within the template sequence of the light chain.

In some embodiments, a variant amino acid within the light chain may reside in one of the framework regions. In some embodiments, a variant amino acid within the VL domain is in the LFR3 region.

In some embodiments, a VL domain of the dual binding antibody comprises LCDRs wherein the amino acid sequence of LCDR1 is set forth in SEQ ID NO: 139, wherein LX1 is selected from the group consisting of N, L, and I, wherein LX2 is selected from the group consisting of L and I, wherein LX3 is selected from the group consisting of S and L; wherein the amino acid sequence of LCDR2 is set forth as D D LX4, wherein LX4 is selected from the group consisting of S and G; and wherein the amino acid sequence of LCDR3 is set forth in SEQ ID NO: 141, wherein LX5 is selected from the group consisting of S and T, wherein LX6 is selected from the group consisting of S and G, and wherein LX7 is selected from the group consisting of H and G. In certain embodiments, the isolated dual binding antibody comprises variant amino acids wherein LCDR1 (SEQ ID NO: 139) wherein LX1 is L, LX2 is I, LX3 is L, LCDR2 (D D LX4, wherein LX4 is selected from the group consisting of S and G), and LCDR3 (SEQ ID NO: 141) wherein LX5 is S, LX6 is S, and LX7 is selected from the group consisting of H and G.

In some embodiments, the dual binding antibody may have a VL domain comprising an LCDR1 (SEQ ID NO: 139) wherein LX1 is L, LX2 is I, and LX3 is L, an LCDR2 (D D LX4, wherein LX4 is S), and LCDR3 (SEQ ID NO: 141) wherein LX5 is S, LX6 is S, and LX7 is G; or an LCDR1 (SEQ ID NO: 139) wherein LX1 is L, LX2 is I, and LX3 is L, an LCDR2 (D D LX4, G wherein LX4 is S), and LCDR3 (SEQ ID NO: 141) wherein LX5 is S, LX6 is S, and LX7 is H, or an LCDR1 (SEQ ID NO: 139) wherein LX1 is L, LX2 is I, and LX3 is L, an LCDR2 (D D LX4, wherein LX4 is G), and LCDR3 (SEQ ID NO: 141) wherein LX5 is S, LX6 is S, and LX7 is G.

Engineered antibody clones having variants in the VL domain as described above, are presented in FIG. 1B.

In some embodiments, disclosed herein is an isolated antibody comprising a heavy chain variable domain (VH) and a light chain variable domain (VL), said VH and VL comprise the amino acid sequences of SEQ ID Nos:209 and 210, SEQ ID Nos:219 and 220, SEQ ID Nos:249 and 250, SEQ ID Nos:337 and 338, SEQ ID Nos:155 and 156, SEQ ID Nos:157 and 158, SEQ ID Nos:4 and 3, SEQ ID Nos:6 and 5, SEQ ID Nos:8 and 7, SEQ ID Nos:10 and 9, SEQ ID Nos:12 and 11, SEQ ID Nos:14 and 13, SEQ ID Nos:16 and 15, SEQ ID Nos:18 and 17, SEQ ID Nos:20 and 19, SEQ ID Nos:22 and 21, SEQ ID Nos:24 and 23, SEQ ID Nos:26 and 25, SEQ ID Nos:28 and 27, SEQ ID Nos:30 and 29, SEQ ID Nos:32 and 31, SEQ ID Nos:34 and 33, SEQ ID Nos:36 and 35, SEQ ID Nos:38 and 37, SEQ ID Nos:40 and 39, SEQ ID Nos:42 and 41, SEQ ID Nos:44 and 43, SEQ ID Nos:46 and 45, SEQ ID Nos:48 and 47, SEQ ID Nos:50 and 49, SEQ ID Nos:52 and 51, or SEQ ID Nos:54 and 53.

In some embodiments, disclosed herein is an isolated antibody comprising a heavy chain variable domain (VH) and a light chain variable domain (VL), said antibody comprising the sequence that is at least 80% identical (e.g., 80%, 85%, 90%, 95%, 98%, or 99% identical) to the sequences set forth in any of SEQ ID Nos:209 and 210, SEQ ID Nos:219 and 220, SEQ ID Nos:249 and 250, SEQ ID Nos:337 and 338, SEQ ID Nos:155 and 156, SEQ ID Nos:157 and 158, SEQ ID Nos:4 and 3, SEQ ID Nos:6 and 5, SEQ ID Nos:8 and 7, SEQ ID Nos:10 and 9, SEQ ID Nos:12 and 11, SEQ ID Nos:14 and 13, SEQ ID Nos:16 and 15, SEQ ID Nos:18 and 17, SEQ ID Nos:20 and 19, SEQ ID Nos:22 and 21, SEQ ID Nos:24 and 23, SEQ ID Nos:26 and 25, SEQ ID Nos:28 and 27, SEQ ID Nos:30 and 29, SEQ ID Nos:32 and 31, SEQ ID Nos:34 and 33, SEQ ID Nos:36 and 35, SEQ ID Nos:38 and 37, SEQ ID Nos:40 and 39, SEQ ID Nos:42 and 41, SEQ ID Nos:44 and 43, SEQ ID Nos:46 and 45, SEQ ID Nos:48 and 47, SEQ ID Nos:50 and 49, SEQ ID Nos:52 and 51, or SEQ ID Nos:54 and 53.

In some embodiments, disclosed herein is an isolated, wherein the antibody comprises a heavy chain variable domain (VH) and a light chain variable domain (VL), said VH and VL comprise the amino acid sequences as shown in Table 10 or Table 1. In some embodiments, disclosed herein is an isolated antibody comprising a heavy chain variable domain (VH) and a light chain variable domain (VL), said antibody comprising the sequence that is at least 80% identical (e.g., 80%, 85%, 90%, 95%, 98%, or 99% identical) to the sequences set forth in Table 10 or Table 1.

In some embodiments, disclosed herein is an isolated antibody comprising a heavy chain variable domain (VH) and a light chain variable domain (VL), wherein
(a) said VH domain comprises the amino acid sequence set forth in SEQ ID NO: 1 with amino acid variants at two or more of positions 52, 99, 100, 101, 102, 103, 104, 105, 106, 107, 108, or 111, or any combination thereof (IMGT positions: 57, 107, 108, 109, 110, 111, 111A, 112A, 112, 113, 114, or 117, or a combination thereof); and
(b) said VL domain comprises the amino acid sequence set forth in SEQ ID NO: 2 with amino acid variants at two or more of positions 26, 27, 31, 51, 56, 77, 92, 93, or 96, or any combination thereof (IMGT positions: 27, 28, 38, 65, 70, 94, 109, 110, or 115, or a combination thereof).

In one embodiment, the isolated dual binding antibody disclosed herein comprises a heavy chain variable domain (VH) and a light chain variable domain (VL), wherein the VH and VL comprise the amino acid sequences of SEQ ID Nos:209 and 210.

In another embodiment, the isolated dual binding antibody disclosed herein comprises a heavy chain variable domain (VH) and a light chain variable domain (VL), wherein the VH and VL comprise the amino acid sequences of SEQ ID Nos:219 and 220.

In another embodiment, the isolated dual binding antibody disclosed herein comprises a heavy chain variable domain (VH) and a light chain variable domain (VL), wherein the VH and VL comprise the amino acid sequences of SEQ ID Nos:249 and 250.

In another embodiment, the isolated dual binding antibody disclosed herein comprises a heavy chain variable domain (VH) and a light chain variable domain (VL), wherein the VH and VL comprise the amino acid sequences of SEQ ID Nos:337 and 338.

In another embodiment, the isolated dual binding antibody disclosed herein comprises a heavy chain variable domain (VH) and a light chain variable domain (VL), wherein the VH and VL comprise the amino acid sequences as shown in Table 1 or Table 10.

In another embodiment, the isolated dual binding antibody disclosed herein comprises VH and VL sequences that are at least 80%, 85%, 90%, 95%, 98%, or 99% identical to the VH and VL sequences disclosed herein.

In some embodiments, an isolated dual binding antibody comprising an antibody antigen-binding domain site comprising a VH domain and a VL domain comprising a combination of VH domain HCDRs and VL domain LCDRs described above. For example, but not limited to, in certain embodiments, a VH domain comprises a set of CDRs, HCDR1, HCDR2, and HCDR3, wherein the amino acid sequence of HCDR1 is set forth in SEQ ID NO: 136; wherein the amino acid sequence of HCDR2 is set forth as: I HX1 Y D G S N K (SEQ ID NO: 142), wherein HX1 is any amino acid; and wherein the amino acid sequence of HCDR3 is set forth as: A R HX2 HX3 HX4 HX5 HX6 HX7 HX8 HX9 HX10 HX11 F D HX12 (SEQ ID NO: 143), wherein XH2, HX3, HX4, HX5, HX6, HX7, HX8, HX9, HX10, HX11, and HX12 are any amino acid; and wherein said VL domain comprises a set of CDRs, LCDR1, LCDR2, and LCDR3, wherein the amino acid sequence of LCDR1 is set forth as LX1, LX2, G S K LX3 V (SEQ ID NO: 144), wherein LX1, LX2, and LX3 are any amino acid; wherein the amino acid sequence of LCDR2 is set forth as D D LX4, wherein LX4 is any amino acid; and wherein the amino acid sequence of LCD3 is set forth as Q V W D LX5 LX6 S D LX7 V V (SEQ ID NO; 146), wherein LX5, LX6, and LX7 are any amino acid.

In some embodiments, a VH domain comprises a set of CDRs, HCDR1, HCDR2, and HCDR3, wherein the amino acid sequence of HCDR1 is set forth in SEQ ID NO: 136, wherein the amino acid sequence of HCDR2 is set forth in SEQ ID NO: 137, wherein HX1 is selected from the group consisting of W and S; wherein the amino acid sequence of HCDR3 is set forth in SEQ ID NO: 138, wherein HX2 is selected from the group consisting of A and S, wherein HX3 is P, wherein HX4 is Q, wherein HX5 is W, wherein HX6 is selected from the group consisting of E, Q, M, L, and V, wherein HX7 is selected from the group consisting of L, W, and Y, wherein HX8 is selected from the group consisting of V and T, wherein HX9 is selected from the group consisting of H, A, S, wherein HX10 is E, wherein HX11 is A, wherein HX12 is selected from the group consisting of I, L, and M; and a VL domain comprises a set of CDRs, HCDR1, HCDR2, and HCDR3, wherein the amino acid sequence of LCDR1 is set forth in SEQ ID NO: 139, wherein LX1 is selected from the group consisting of N, L, and I, wherein LX2 is selected from the group consisting of L and I, wherein LX3 is selected from the group consisting of S and L; wherein the amino acid sequence of LCDR2 is set forth as D D LX4, wherein LX4 is selected from the group consisting of S and G; wherein the amino acid sequence of LCDR3 is set forth in SEQ ID NO: 141, wherein LX5 is selected from the group consisting of S and T, wherein LX6 is selected from the group consisting of S and G, and wherein LX7 is selected from the group consisting of H and G.

In certain embodiments, the isolated dual binding antibody comprises a VH domain comprising a set of CDRs, HCDR1, HCDR2, and HCDR3, wherein the amino acid sequence of HCDR1 is set forth in SEQ ID NO: 136, wherein the amino acid sequence of HCDR2 is set forth in SEQ ID NO: 137, wherein HX1 is W, wherein the amino acid sequence of HCDR3 is set forth in SEQ ID NO: 138, wherein HX2 is selected from the group consisting of A and S, HX3 is P, HX4 is Q, HX5 is W, HX6 is selected from the group consisting of E and M, HX7 is selected from the group consisting of L and W, HX8 is selected from the group consisting of V and T, HX9 is selected from the group consisting of H and A, HX10 is E, HX11 is A, HX12 is selected from the group I and L, and comprising a VL domain comprising a set of CDRs, LCDR1, LCDR2, and LCDR3, wherein the amino acid sequence of LCDR1 is set forth in SEQ ID NO: 139 wherein LX1 is L, LX2 is I, LX3 is L, wherein the amino acid sequence of LCDR2 is set forth as D D LX4, wherein LX4 is selected from the group consisting of S and G, wherein the amino acid sequence of LCDR3 is set forth in SEQ ID NO: 141 wherein LX5 is S, LX6 is S, and LX7 is selected from the group consisting of H and G.

In some embodiments, the dual binding antibody comprises a VH domain comprising a set of CDRs, HCDR1, HCDR2, and HCDR3, and a VL domain comprising a set of CDRs, LCDR1, LCDR2, and LCDR3, wherein the amino acid sequences of each CDR are as set forth in FIGS. 1A and 1B for the clones set forth there, for example but not limited to: Clone C2: HCDR1 (SEQ ID NO: 136), HCDR2 (SEQ ID NO: 137) wherein HX1 is W, HCDR3 (SEQ ID NO: 138) wherein HX2 is A, HX3 is P, HX4 is Q, HX5 is W, HX6 is E, HX7 is L, HX8 is T, HX9 is A, HX10 is E, HX11 is A, HX12 is I, LCDR1 (SEQ ID NO: 139) wherein LX1 is L, LX2 is I, LX3 is L, LCDR2 (D D LX4, wherein LX4 is S), and LCDR3 (SEQ ID NO: 141) wherein LX5 is S, LX6 is S, and LX7 is G; Clone C6: HCDR1 (SEQ ID NO: 136), HCDR2 (SEQ ID NO: 137) wherein HX1 is W, HCDR3 (SEQ ID NO: 138) wherein HX2 is A, HX3 is P, HX4 is Q, HX5 is W, HX6 is M, HX7 is L, HX8 is V, HX9 is A, HX10 is E, HX11 is A, HX12 is L, LCDR1 (SEQ ID NO: 139) wherein LX1 is L, LX2 is I, LX3 is L, LCDR2 (D D LX4, wherein LX4 is S), and LCDR3 (SEQ ID NO: 141) wherein LX5 is S, LX6 is S, and LX7 is H; or Clone C9: HCDR1 (SEQ ID NO: 136), HCDR2 (SEQ ID NO: 137) wherein HX1 is W, HCDR3 (SEQ ID NO: 138) wherein HX2 is S, HX3 is P, HX4 is Q, HX5 is W, HX6 is E, HX7 is W, HX8 is V, HX9 is H, HX10 is E, HX11 is A, HX12 is L, LCDR1 (SEQ ID NO: 139) wherein LX1 is L, LX2 is I, LX3 is L, LCDR2 (D D LX4, wherein LX4 is G), and LCDR3 (SEQ ID NO: 141) wherein LX5 is S, LX6 is S, and LX7 is G.

In some embodiments, the dual binding antibody comprises a set of HCDRs as disclosed herein, and any VL domain. In some embodiments, the dual binding antibody comprises a set of LCDRs as disclosed herein, and any VH domain. In some embodiments, the dual binding antibody comprises a paired set of HCDRs-LCDRs, as disclosed herein.

In certain embodiments, the dual binding antibody comprising a VH domain comprising HCDRs as described herein may be encoded by a nucleic acid construct. In certain embodiments, the dual binding antibody comprising a VL domain comprising LCDRS as described herein may be encoded by a nucleic acid construct. In certain embodiments, the dual binding antibody comprising a VH domain comprising HCDRs and a VL domain comprising HCDRs as described herein, may be encoded by a nucleic acid construct.

In certain embodiments, the dual binding antibody comprising a VH domain comprising HCDRs as described herein, may be encoded by a nucleic acid construct. In certain embodiments, the dual binding antibody comprising a VL domain comprising LCDRs as described herein may be encoded by a nucleic acid construct. In certain embodiments, the dual binding antibody comprising a VH domain comprising HCDRs and a VL domain comprising HCDRs as described herein, may be encoded by a nucleic acid construct.

In certain embodiments, the dual binding antibody comprising a VH domain comprising HCDRs as described herein, may be comprised within a library of immunoglobulins. In certain embodiments, the dual binding antibody comprising a VL domain comprising LCDRs as described herein may be comprised within a library of immunoglobulins. In certain embodiments, the dual binding antibody comprising a VH domain comprising HCDRs and a VL domain comprising HCDRs as described herein, may be comprised within a library of immunoglobulins.

In certain embodiments, the dual binding antibody comprising a VH domain comprising HCDRs as described herein, may be produced by expressing a nucleic acid construct comprising a nucleic acid sequence encoding the HCDRs from a host cell and isolating the antibody. In certain embodiments, the dual binding antibody comprising a VL domain comprising LCDRs as described herein may be produced by expressing a nucleic acid construct comprising a nucleic acid sequence encoding the LCDRs from a host cell and isolating the antibody. In certain embodiments, the dual binding antibody comprising a VH domain comprising HCDRs and a VL domain comprising HCDRs as described herein, may be produced by expressing a nucleic acid construct comprising a nucleic acid sequence encoding the HCDRs and LCDRs from a host cell and isolating the antibody.

In certain embodiments, the dual binding antibody comprising a VH domain comprising HCDRs as described herein may be administered in a method of treating a subject in need, wherein said subject suffers from a disease or condition comprising an allergic or respiratory condition, an inflammatory and/or autoimmune condition of the skin or gastrointestinal organs; scleroderma; or tumors or cancers including Hodgkin's lymphoma. In certain embodiments, the dual binding antibody comprising a VL domain comprising LCDRs as described herein may be administered in a method of treating a subject in need, wherein said subject suffers from a disease or condition comprising an allergic or respiratory condition, an inflammatory and/or autoimmune condition of the skin or gastrointestinal organs; scleroderma; or tumors or cancers including Hodgkin's lymphoma. In certain embodiments, the dual binding antibody comprising a VH domain comprising HCDRs and a VL domain comprising LCDRs as described herein, may be administered in a method of treating a subject in need, wherein said subject suffers from a disease or condition comprising an allergic or respiratory condition, an inflammatory and/or autoimmune condition of the skin or gastrointestinal organs; scleroderma; or tumors or cancers including Hodgkin's lymphoma.

In some embodiments, an antibody comprising a heavy chain variable region amino acid sequence set forth in SEQ ID NO: 1 or a light chain variable region amino acid sequence set forth in SEQ ID NO: 2, or the combination thereof, does not bind an IL-13 epitope. Accordingly, the dual binding antibodies described herein are engineered to comprise a binding region not previously present in the antibody. In other words, the dual binding antibodies comprise “re-epitoped” antibodies. As used throughout, the term “engineered” and “re-epitoped” may in certain embodiments, be used interchangeably having all the same qualities and meanings. In some embodiments, a “re-epitoped” antibody comprises improved binding compared to available antibodies. In some embodiments, a “re-epitoped” antibody comprises improved association and dissociation constants (Kₒₙ and K_{off}), compared to the parent antibodies. In some embodiments, a “re-epitoped” antibody comprises improved stability compared with the parent antibodies. In certain embodiments, incorporating variant amino acid residues in at least two of the unique set of 21 variant sites within the CDRs and FR of the VH domain and VL domain, as described herein, results in a “re-epitoped” dual binding antibody comprising improved characteristics compared with the parent antibodies. These re-epitoped antibodies may provide advantageous characteristics.

A skilled artisan would recognize that a “Fv” with regard to an antibody encompasses the smallest fragment of the antibody to bear the complete antigen binding site. An Fv fragment consists of the variable region of a single light chain (VL) bound to the variable region of a single heavy chain (VH).

A skilled artisan would recognize that a “single-chain Fv antibody” or “scFv” with regard to an antibody encompasses an engineered antibody consisting of a VL domain and a VH domain connected to one another directly or via a peptide linker sequence. The skilled artisan would appreciate that a linker, in some embodiments, may comprise a linear amino acid sequence. In some embodiments, the linear amino acid sequence (“linker”) comprises an enzyme cleavage site and may, in certain embodiments, be termed a “cleavable linker” or a “linker” or a “cleavable peptide”. In some embodiments, a linker may be a cleavable linker. In some embodiments, a linker may be a non-cleavable. In some embodiments, a linker sequence is set forth in SEQ ID NO: 147 (GGGGSGGGGSGGGGS; SEQ ID NO: 147).

In some embodiments, peptide linker sequences contain, for example, Gly, Asn, and or Ser residues, in various combinations. Other near neutral amino acids, such as Thr and Ala, may also be included in the linker sequence.

Other amino acid sequences which may be usefully employed as linkers include those disclosed in Maratea et al., Gene 40:39 46 (1985); Murphy et al., Proc. Natl. Acad. Sci. USA 83:8258 8262 (1986); U.S. Pat. Nos. 4,935,233 and 4,751,180; Chaudhary et al., 1990, Proc. Natl. Acad. Sci. U.S.A. 87:1066-1070; Bird et al., 1988, Science 242:423-426, incorporated herein in their entirety.

In some embodiments, coding sequences of VH and VL domains of the dual binding antibody or fragment thereof can be fused directly without any junctional amino acids or by using a flexible polylinker composed.

A peptide linker, in certain embodiments, is designed to enable the correct interaction between two beta-sheets forming the variable region of the single chain antibody. Any suitable linkers can be used to make an indirect link, such as without limitation, peptide linker, polymer linker, and chemical linker. In certain embodiments, the covalent link is an indirect link through a peptide linker.

In some embodiments, an antibody comprises a mutated immunoglobulin. Examples of mutated immunoglobulins include but are not limited to an IgG that does not bind antibody-dependent cellular cytotoxicity (ADCC) components. IgG comprising L₂₃₄A/L₂₃₅A (LALA) mutations cannot bind the Fc receptor (See, Xu D, Alegre M L, Varga S S, Rothermel A L, Collins A M, Pulito V L, et al. In vitro characterization of five humanized OKT3 effector function variant antibodies. Cell Immunol. (2000) 200:16-26.). In some embodiments, the dual binding antibody comprises an IgG comprising the L₂₃₄A/L₂₃₅A (LALA) mutations. In some embodiments, the dual binding antibody comprises an IgG comprising the L234A/L235A/P329G (LALAPG) mutations. (See, Wilkenson et al (2021) Fc-engineered antibodies with immune effector functions completely abolished. PLOS ONE I https://doi.org/10.1371/journal.pone.0260954) The mutations as numbered here are based on an EU numbering convention used for the constant region, (See, Xu D, Alegre M L, Varga S S, Rothermel A L, Collins A M, Pulito V L, et al. In vitro characterization of five humanized OKT3 effector function variant antibodies. Cell Immunol. (2000) 200:16-26. 10.1006/cimm.2000.1617).

In some embodiments, a heavy chain (HC) of an IgG comprising a LALA mutation has the amino acid sequence set forth in SEQ ID NO: 410. In some embodiments, a heavy chain of an IgG comprising a LALAPG mutation has the amino acid sequence set forth in SEQ ID NO: 408.

In some embodiments, an IgG comprising an Fc mutation in the HC resulting in reduced binding to an Fc receptor and an IgG that does not bind antibody-dependent cellular cytotoxicity (ADCC) components, the IgG comprises a HC having the amino acids set forth in SEQ ID NO: 410 and a light chain (LC) having the amino acid sequence set forth in SEQ ID NO: 409. In some embodiments, an IgG comprising an Fc mutation in the HC resulting in reduced binding to an Fc receptor and in an IgG that does not bind antibody-dependent cellular cytotoxicity (ADCC) components, the IgG comprises a HC having the amino acids set forth in SEQ ID NO: 408 and a light chain (LC) having the amino acid sequence set forth in SEQ ID NO: 409.

In some embodiments, a mutated IgG comprises an IgG1, wherein the Fc region is engineered. In some embodiments, a mutated IgG comprises an IgG2, wherein the Fc region is engineered. In some embodiments, a mutated IgG comprises an IgG3, wherein the Fc region is engineered. In some embodiments, a mutated IgG comprises an IgG4, wherein the Fc region is engineered. In certain embodiments, mutations within an Fc region of an antibody abolishes immune effector functions of the antibody.

In some embodiments, an isolated dual binding antibody comprises an IgG, an Fv, an scFv, an Fab, an F(ab′)2, a minibody, a diabody, or a triabody. In some embodiments, an isolated dual binding antibody comprises an IgG, wherein said IgG is IgG1, IgG2, IgG3, or IgG4.

In some embodiments, an isolated dual binding antibody comprises a mutated IgG, wherein said mutant IgG is unable to bind to antibody-dependent cellular cytotoxicity components.

In some embodiments, the dual binding antibody described herein comprises an IgG immunoglobulin. In some embodiments, the dual binding antibody described herein comprises an IgG1 immunoglobulin, an IgG2 immunoglobulin, an IgG3 immunoglobulin, or an IgG4 immunoglobulin. In some embodiments, the dual binding antibody comprises an IgG1 immunoglobulin. In some embodiments, the dual binding antibody comprises an IgG2 immunoglobulin. In some embodiments, the dual binding antibody comprises an IgG3 immunoglobulin. In some embodiments, the dual binding antibody comprises an IgG4 immunoglobulin. In some embodiments, the dual binding antibody comprises an IgG1 immunoglobulin or an IgG4 immunoglobulin.

In some embodiments, the dual binding antibody described herein comprises an Fab immunoglobulin fragment. In some embodiments, the dual binding antibody described herein comprises an F(ab′)₂ immunoglobulin fragment. In some embodiments, the dual binding antibody described herein comprises an Fv immunoglobulin construct. In some embodiments, the dual binding antibody described herein comprises an scFv immunoglobulin construct. In some embodiments, the dual binding antibody described herein comprises a minibody immunoglobulin construct comprising a pair of single-chain Fv fragments which are linked via CH3 domains.

In some embodiments, the dual binding antibody described herein comprises a diabody immunoglobulin construct. In some embodiments, a diabody immunoglobulin construct comprises a heavy chain variable (VH) and light chain variable (VL) regions connected by a small peptide linker. In some embodiments, a diabody immunoglobulin construct comprises single-chain (Fv) 2 in which two scFv fragments are covalently linked to each other. In some embodiments, the dual binding antibody described herein comprises a diabody immunoglobulin construct comprising three scFv fragments covalently linked to each other. Diabodies have been shown in the art to have dissociation constants up to 40-fold lower than corresponding scFvs, meaning that they have a much higher affinity to their target. Consequently, use of a diabody in a method of use as described below, could result in much lower dosing of a diabody or triabody, than an IgG comprising the same VH and VL domains.

In some embodiments, the dual binding antibody comprising a linker or linkers between binding components, for example but not limited to between a VH and a VL in an scFv, a minibody, a diabody, a triabody, or a tetrabody. In some embodiments, the dual binding antibody does not comprise a linker or linkers between binding components, for example but not limited to between a VH and a VL in an scFv, a minibody, a diabody, a triabody, or a tetrabody. In some embodiments, a linker may comprise a single amino acid. In some embodiments, a linker comprises any known linker in the art. In some embodiments, a linker comprises the amino acid sequence set forth in SEQ ID NO: 147.

A skilled artisan would appreciate that the term “variant” encompasses a polypeptide differing from a specifically recited polypeptide sequences, for example the amino acid sequences set forth in SEQ ID NO: 1 and SEQ ID NO: 2, by single or multiple amino acid insertions, deletions, and/or substitutions, created using, e.g., recombinant DNA techniques. Variants of the antigen binding molecules disclosed herein include antigen binding molecules wherein one or several of the amino acid residues are modified by at last one substitution, addition and/or deletion in such manner that the antigen binding affinity is newly created in the antigen binding molecules.

The dual binding portion of the antibodies described herein comprises an immunoglobulin heavy chain variable region and an immunoglobulin light chain variable region (VH and VL, respectively), wherein the amino acid sequence set forth in SEQ ID NO: 1 comprises a VH template and the amino acid sequence set forth in SEQ ID NO: 2 comprises a VL template, and the dual binding region comprises at least two variants within the VH template sequence, or within the VL template sequence, or a combination thereof.

A skilled artisan would appreciate that an “isolated dual binding antibody”, in certain embodiments, encompasses an antibody that (1) is free of at least some other proteins with which it would typically be found in nature or with which it would typically be found during synthesis thereof, (2) is essentially free of other non-identical binding antibodies from the same source, (3) may be expressed recombinantly by a cell, (4) has been separated from at least about 50 percent of polynucleotides, lipids, carbohydrates, or other materials with which it is associated in during synthesis, or (5) does not occur in nature, or a combination thereof. Such an isolated antibody may be encoded by genomic DNA, cDNA, mRNA or other RNA, or may be of synthetic origin, or any combination thereof. In certain embodiments, the isolated antibody is substantially free from proteins or polypeptides or other contaminants that would interfere with its use (therapeutic, diagnostic, prophylactic, research or otherwise). As used throughout, the terms “dual antibody” and “dual binding antibody” may be used interchangeably having all the same meanings and qualities.

In some embodiments, disclosed herein is an isolated dual binding antibody comprising a heavy chain variable region comprising the amino acid sequence set forth in SEQ ID NO: 1 with at least one amino acid variant at any of positions 52, 99, 100, 101, 102, 103, 104, 105, 106, 107, 108, or 111, or any combination thereof (IMGT numbering of heavy chain variable region variant positions: 57, 107, 108, 109, 110, 111, 111A, 112A, 112, 113, 114, or 117, or a combination thereof); a light chain variable region comprising the amino acid sequence set forth in SEQ ID NO: 2 with at least one amino acid variant at any of positions 26, 27, 31, 51, 56, 77, 92, 93, or 96, or any combination thereof (IMGT numbering of light chain variable region variant positions: 27, 28, 38, 65, 70, 94, 109, 110, or 115, or a combination thereof); or a combination of the variant heavy chain variable region and the variant light chain variable region; wherein the total number of variant positions in said heavy chain variable region, said light chain variable region, or said combination thereof, is at least 2.

IMGT® is the international ImMunoGeneTics information system®, (See, Nucleic Acids Res. 2015 January; 43(Database issue): D413-22. Doi: 10.1093/nar/gku1056. Epub 2014 Nov. 5 Free article. PMID: 25378316 LIGM:441 and Dev Comp Immunol. 2003 January; 27(1):55-77). IMGT is a unique numbering system for immunoglobulin and T cell receptor variable domains and Ig superfamily V-like domains, (Lefranc MP1, Pommié C, Ruiz M, Giudicelli V, Foulquier E, Truong L, Thouvenin-Contet V, Lefranc G. Dev Comp Immunol 27: 55-77. (2003)). IMGT® presents a uniform numbering system for these IG and TcR variable domain sequences, based on aligning 5 or more000 IG and TcR variable region sequences, taking into account and combining the Kabat definition of FRs and CDRs, structural data, and Chothia's characterization of the hypervariable loops. IMGT is considered a universal numbering scheme for antibodies well known in the art.

In describing variant amino acid positions present in the VH and VL domains, in some embodiments the IMGT numbering is used. In some embodiments, variant amino acid positions are presented as the specific positions within a given sequence, for example but not limited to SEQ ID NO: 1 and SEQ ID NO: 2. In some embodiments, variant amino acid positions are identified by both the specific positions within a given SEQ ID NO: sequence and the IMGT numbering system. A skilled artisan would recognize that the actual amino acid position number of an amino acid identified by position number relative to a SEQ ID NO: may differ from the IMGT numbering system, yet the residue identified is identical. For example, but not limited to the amino acid residue at position 106 of SEQ ID NO: 1 is the identical residue identified at position 112 by the IMGT numbering system. The skilled artisan would recognize that while the same amino acid residue may be identified as having different positions depending what system is used, the amino acid residue's location and identity within a contiguous amino acid sequence is clear.

In some embodiments, disclosed herein is an isolated dual binding antibody comprising a heavy chain variable region comprising the amino acid sequence set forth in SEQ ID NO: 1 with at least one amino acid variants at any of positions 52, 99, 100, 101, 102, 103, 104, 105, 106, 107, 108, or 111, or any combination thereof (IMGT numbering of heavy chain variable region variant positions: 57, 107, 108, 109, 110, 111, 111A, 112A, 112, 113, 114, or 117, or a combination thereof), and a light chain variable region comprising the amino acid sequence set forth in SEQ ID NO: 2, wherein optionally said amino acid sequence SEQ ID NO: 2 comprises at least one variant amino acid, wherein the total number of variant positions in said heavy chain variable region, light chain variable region, or a combination thereof is at least 2. In some embodiments, disclosed herein is an isolated dual binding antibody comprising a heavy chain variable region comprising the amino acid sequence set forth in SEQ ID NO: 1 with at least two amino acid variants at any of positions 52, 99, 100, 101, 102, 103, 104, 105, 106, 107, 108, or 111, or any combination thereof (IMGT numbering of heavy chain variable region variant positions: 57, 107, 108, 109, 110, 111, 111A, 112A, 112, 113, 114, or 117, ora combination thereof), and any light chain variable region.

The amino acid sequences of many light chain variable regions, in and of themselves, are known as such in the art. The skilled person would be able to use such known sequences, and in conjunction with the heavy chain variable regions described herein, analyze for dual binding using routine methodologies and techniques well known in the art (See for example but not limited to, Example 1 below).

In some embodiments, disclosed herein is an isolated dual binding antibody comprising a light chain variable region comprising the amino acid sequence set forth in SEQ ID NO: 2 with at least one amino acid variants at any of positions 26, 27, 31, 51, 56, 77, 92, 93, or 96, or any combination thereof (IMGT numbering of light chain variable region variant positions: 27, 28, 38, 65, 70, 94, 109, 110, or 115, or a combination thereof), and a heavy chain variable region comprising the amino acid sequence set forth in SEQ ID NO: 1, wherein optionally said amino acid sequence SEQ ID NO: 1 comprises at least one variant amino acid, wherein the total number of variant positions in said heavy chain variable region, said light chain variable region, or a combination thereof is at least 2. In some embodiments, disclosed herein is an isolated dual binding antibody comprising a light chain variable region comprising the amino acid sequence set forth in SEQ ID NO: 2 with at least two amino acid variants at any of positions 26, 27, 31, 51, 56, 77, 92, 93, or 96, or any combination thereof (IMGT numbering of light chain variable region variant positions: 27, 28, 38, 65, 70, 94, 109, 110, or 115, or a combination thereof), and any heavy chain variable region.

The amino acid sequences of many heavy chain variable regions, in and of themselves, are known as such in the art. The skilled person would be able to use such known sequences, and in conjunction with the light chain variable regions described herein, analyze for dual binding using routine methodologies and techniques well known in the art (See for example but not limited to, Example 1 below).

In some embodiments, a VH domain described herein comprises the amino acid sequence set forth in SEQ ID NO: 1 with at least one amino acid variants at any position. In some embodiments, a VH comprising the amino acid sequence set forth in SEQ ID NO: 1 comprises at least two amino acid variants at any position. In some embodiments, a VH comprising the amino acid sequence set forth in SEQ ID NO: 1 comprises at least between 1-10 amino acid variants at any position. In some embodiments, a VH comprising the amino acid sequence set forth in SEQ ID NO: 1 comprises 1-5 amino acid variants at any position. In some embodiments, a VH comprising the amino acid sequence set forth in SEQ ID NO: 1 comprises 1, 2, 3, 4, 5, 6, 7, 8, 9, 10, or more amino acid variants at any position.

In some embodiments, a VL domain described herein comprises the amino acid sequence set forth in SEQ ID NO: 2 with at least one amino acid variants at any position. In some embodiments, a VL comprising the amino acid sequence set forth in SEQ ID NO: 2 comprises at least two amino acid variants at any position. In some embodiments, a VL comprising the amino acid sequence set forth in SEQ ID NO: 2 comprises at least between 1-10 amino acid variants at any position. In some embodiments, a VL comprising the amino acid sequence set forth in SEQ ID NO: 2 comprises 1-5 amino acid variants at any position. In some embodiments, a VL comprising the amino acid sequence set forth in SEQ ID NO: 2 comprises 1, 2, 3, 4, 5, 6, 7, 8, 9, 10, or more amino acid variants at any position.

In some embodiments, a VH domain and a VL domain described herein comprising the amino acid sequences set forth in SEQ ID NO: 1 and SEQ ID NO: 2, respectfully have a combined number of variant positions of at least 2. In some embodiments, a VH domain and a VL domain described herein comprising the amino acid sequences set forth in SEQ ID NO: 1 and SEQ ID NO: 2, respectfully have a combined number of variant positions of between 2-20. In some embodiments, a VH domain and a VL domain described herein comprising the amino acid sequences set forth in SEQ ID NO: 1 and SEQ ID NO: 2, respectfully have a combined number of variant positions of at least 2, 3, 4, 5, 6, 7, 8, 9, 10, 11, 12, 13, 14, 15, 16 17, 18, 19, or 20. In some embodiments, a VH domain and a VL domain described herein comprising the amino acid sequences set forth in SEQ ID NO: 1 and SEQ ID NO: 2, respectfully have a combined number of variant positions of more than 20.

In certain embodiments, dual antibody binding regions, as described herein include a heavy chain and a light chain CDR set, respectively, interposed between a heavy chain and a light chain framework region (FR) set which provide support to the CDRs and define the spatial relationship of the CDRs relative to each other. As used herein, the term “CDR set” refers to the three hypervariable regions of a heavy or light chain variable region. Proceeding from the N-terminus of a heavy or light chain polypeptide, these regions are denoted as “CDR1,” “CDR2,” and “CDR3” respectively. An antigen binding site, therefore, includes six CDRs, comprising the CDR set from each of a heavy and a light chain variable region. Crystallographic analysis of a number of antigen-antibody complexes has demonstrated that the amino acid residues of CDRs form extensive contact with a bound antigen, wherein the most extensive antigen contact is with the heavy chain CDR3. Thus, the CDR regions are primarily responsible for the specificity of an antigen binding site.

As used herein, the term “FR set” refers to the four flanking amino acid sequences which frame the CDRs of a CDR set of a heavy or light chain variable region. Some FR residues may contact bound antigen; however, FRs are primarily responsible for folding the variable region into the antigen-binding site, particularly the FR residues directly adjacent to the CDRs. Within FRs, certain amino residues and certain structural features are very highly conserved. In this regard, all variable region sequences contain an internal disulfide loop of around 90 amino acid residues. When the variable regions fold into a binding-site, the CDRs are displayed as projecting loop motifs which form an antigen-binding surface. It is generally recognized that there are conserved structural regions of FRs, which influence the folded shape of the CDR loops into certain “canonical” structures—regardless of the precise CDR amino acid sequence. Further, certain FR residues are known to participate in non-covalent interdomain contacts which stabilize the interaction of the antibody heavy and light chains.

In some embodiments, an at least one variant in said VH comprises a variant amino acid in a CDR region. In some embodiments, an at least one variant in said VL comprises a variant amino acid in a CDR region. In some embodiments, an at least one variant in said VH comprises a variant amino acid in a FR region. In some embodiments, an at least one variant in said VL comprises a variant amino acid in a FR region. In some embodiments, an at least two variants in said VH comprises variant amino acids in a CDR region, an FR region, or both. In some embodiments, an at least two variants in said VL comprises variant amino acids in a CDR region, an FR region, or both. In some embodiments, variant positions in the VH include variants in at least one CDR and at least one FR region. In some embodiments, variant positions in the VL include variants in at least one CDR and at least one FR region.

In some embodiments, a VH comprising the amino acid sequence set forth in SEQ ID NO: 1 comprises an amino acid variant at any of positions 52, 99, 100, 101, 102, 103, 104, 105, 106, 107, 108, or 111, or any combination thereof (IMGT numbering of heavy chain variable region variant positions: 57, 107, 108, 109, 110, 111, 111A, 112A, 112, 113, 114, or 117, or a combination thereof). In some embodiments, a VH comprising the amino acid sequence set forth in SEQ ID NO: 1 comprises an amino acid variant at position 52 (IMGT position 57). In some embodiments, a VH comprising the amino acid sequence set forth in SEQ ID NO: 1 comprises an amino acid variant at position 99 (IMGT position 107). In some embodiments, a VH comprising the amino acid sequence set forth in SEQ ID NO: 1 comprises an amino acid variant at position 100 (IMGT position 108). In some embodiments, a VH comprising the amino acid sequence set forth in SEQ ID NO: 1 comprises an amino acid variant at position 101 (IMGT position 109). In some embodiments, a VH comprising the amino acid sequence set forth in SEQ ID NO: 1 comprises an amino acid variant at position 102 (IMGT position 110). In some embodiments, a VH comprising the amino acid sequence set forth in SEQ ID NO: 1 comprises an amino acid variant at position 103 (IMGT position 111). In some embodiments, a VH comprising the amino acid sequence set forth in SEQ ID NO: 1 comprises an amino acid variant at position 104 (IMGT position 111A). In some embodiments, a VH comprising the amino acid sequence set forth in SEQ ID NO: 1 comprises an amino acid variant at position 105 (IMGT position 112A). In some embodiments, a VH comprising the amino acid sequence set forth in SEQ ID NO: 1 comprises an amino acid variant at position 106 (IMGT position 112). In some embodiments, a VH comprising the amino acid sequence set forth in SEQ ID NO: 1 comprises an amino acid variant at position 107 (IMGT position 113). In some embodiments, a VH comprising the amino acid sequence set forth in SEQ ID NO: 1 comprises an amino acid variant at position 108 (IMGT position 114). In some embodiments, a VH comprising the amino acid sequence set forth in SEQ ID NO: 1 comprises an amino acid variant at position 111 (IMGT position 117).

In some embodiments, a VH comprising the amino acid sequence set forth in SEQ ID NO: 1 comprises an amino acid variant at position 52 (IMGT position 57) and between 1-3 additional variant amino acids. In some embodiments, a VH comprising the amino acid sequence set forth in SEQ ID NO: 1 comprises an amino acid variant at position 99 (IMGT position 107) and between 1-3 additional variant amino acids. In some embodiments, a VH comprising the amino acid sequence set forth in SEQ ID NO: 1 comprises an amino acid variant at position 100 (IMGT position 108) and between 1-3 additional variant amino acids. In some embodiments, a VH comprising the amino acid sequence set forth in SEQ ID NO: 1 comprises an amino acid variant at position 101 (IMGT position 109) and between 1-3 additional variant amino acids. In some embodiments, a VH comprising the amino acid sequence set forth in SEQ ID NO: 1 comprises an amino acid variant at position 102 (IMGT position 110) and between 1-3 additional variant amino acids. In some embodiments, a VH comprising the amino acid sequence set forth in SEQ ID NO: 1 comprises an amino acid variant at position 103 (IMGT position 111) and between 1-3 additional variant amino acids. In some embodiments, a VH comprising the amino acid sequence set forth in SEQ ID NO: 1 comprises an amino acid variant at position 104 (IMGT position 111A) and between 1-3 additional variant amino acids. In some embodiments, a VH comprising the amino acid sequence set forth in SEQ ID NO: 1 comprises an amino acid variant at position 105 (IMGT position 112A) and between 1-3 additional variant amino acids. In some embodiments, a VH comprising the amino acid sequence set forth in SEQ ID NO: 1 comprises an amino acid variant at position 106 (IMGT position 112) and between 1-3 additional variant amino acids. In some embodiments, a VH comprising the amino acid sequence set forth in SEQ ID NO: 1 comprises an amino acid variant at position 107 (IMGT position 113) and between 1-3 additional variant amino acids. In some embodiments, a VH comprising the amino acid sequence set forth in SEQ ID NO: 1 comprises an amino acid variant at position 108 (IMGT position 114) and between 1-3 additional variant amino acids. In some embodiments, a VH comprising the amino acid sequence set forth in SEQ ID NO: 1 comprises an amino acid variant at position 111 (IMGT position 117) and between 1-3 additional variant amino acids.

In some embodiments, a VH comprising the amino acid sequence set forth in SEQ ID NO: 1 comprises an amino acid variant at positions 105 and 106 (IMGT positions 112A and 112). In some embodiments, a VH comprising the amino acid sequence set forth in SEQ ID NO: 1 comprises an amino acid variant at positions 106 and 111 (IMGT positions 112 and 117). In some embodiments, a VH comprising the amino acid sequence set forth in SEQ ID NO: 1 comprises an amino acid variant at positions 103 and 106 (IMGT positions 111 and 112). In some embodiments, a VH comprising the amino acid sequence set forth in SEQ ID NO: 1 comprises an amino acid variant at positions 104 and 106 (IMGT positions 111A and 112). In some embodiments, a VH comprising the amino acid sequence set forth in SEQ ID NO: 1 comprises an amino acid variant at positions 104, 106, and 111 (IMGT positions 111A, 112, and 117). In some embodiments, a VH comprising the amino acid sequence set forth in SEQ ID NO: 1 comprises an amino acid variant at positions 105, 106, and 111 (IMGT positions 112A, 112, and 117). In some embodiments, a VH comprising the amino acid sequence set forth in SEQ ID NO: 1 comprises an amino acid variant at positions 103, 106, and 111 (IMGT positions 111, 112, and 117). In some embodiments, a VH comprising the amino acid sequence set forth in SEQ ID NO: 1 comprises an amino acid variant at positions 99, 104, and 111 (IMGT positions 107, 111A, and 117). In some embodiments, a VH comprising the amino acid sequence set forth in SEQ ID NO: 1 comprises an amino acid variant at positions 52, 99, 104, and 111 (IMGT positions 57, 107, 111A, and 117).

In some embodiments, a VL comprising the amino acid sequence set forth in SEQ ID NO: 2 comprises an amino acid variant at any of positions 26, 27, 31, 51, 56, 77, 92, 93, or 96, or any combination thereof (IMGT numbering of light chain variable region variant positions: 27, 28, 38, 65, 70, 94, 109, 110, or 115, or a combination thereof). In some embodiments, a VL comprising the amino acid sequence set forth in SEQ ID NO: 2 comprises an amino acid variant at position 26 (IMGT position 27). In some embodiments, a VL comprising the amino acid sequence set forth in SEQ ID NO: 2 comprises an amino acid variant at position 27 (IMGT position 28). In some embodiments, a VL comprising the amino acid sequence set forth in SEQ ID NO: 2 comprises an amino acid variant at position 31 (IMGT position 38). In some embodiments, a VL comprising the amino acid sequence set forth in SEQ ID NO: 2 comprises an amino acid variant at position 51 (IMGT position 65). In some embodiments, a VL comprising the amino acid sequence set forth in SEQ ID NO: 2 comprises an amino acid variant at position 56 (IMGT position 70). In some embodiments, a VL comprising the amino acid sequence set forth in SEQ ID NO: 2 comprises an amino acid variant at position 77 (IMGT position 94). In some embodiments, a VL comprising the amino acid sequence set forth in SEQ ID NO: 2 comprises an amino acid variant at position 92 (IMGT position 109). In some embodiments, a VL comprising the amino acid sequence set forth in SEQ ID NO: 2 comprises an amino acid variant at position 93 (IMGT position 110). In some embodiments, a VL comprising the amino acid sequence set forth in SEQ ID NO: 2 comprises an amino acid variant at position 96 (IMGT position 115).

In some embodiments, a VL comprising the amino acid sequence set forth in SEQ ID NO: 2 comprises an amino acid variant at position 26 (IMGT position 27) and between 1-7 additional variant amino acids. In some embodiments, a VL comprising the amino acid sequence set forth in SEQ ID NO: 2 comprises an amino acid variant at position 27 (IMGT position 28) and between 1-7 additional variant amino acids. In some embodiments, a VL comprising the amino acid sequence set forth in SEQ ID NO: 2 comprises an amino acid variant at position 31 (IMGT position 38) and between 1-7 additional variant amino acids. In some embodiments, a VL comprising the amino acid sequence set forth in SEQ ID NO: 2 comprises an amino acid variant at position 51 (IMGT position 65) and between 1-7 additional variant amino acids. In some embodiments, a VL comprising the amino acid sequence set forth in SEQ ID NO: 2 comprises an amino acid variant at position 56 (IMGT position 70) and between 1-7 additional variant amino acids. In some embodiments, a VL comprising the amino acid sequence set forth in SEQ ID NO: 2 comprises an amino acid variant at position 77 (IMGT position 94) and between 1-7 additional variant amino acids. In some embodiments, a VL comprising the amino acid sequence set forth in SEQ ID NO: 2 comprises an amino acid variant at position 92 (IMGT position 109) and between 1-7 additional variant amino acids. In some embodiments, a VL comprising the amino acid sequence set forth in SEQ ID NO: 2 comprises an amino acid variant at position 93 (IMGT position 110) and between 1-7 additional variant amino acids. In some embodiments, a VL comprising the amino acid sequence set forth in SEQ ID NO: 2 comprises an amino acid variant at position 96 (IMGT position 115) and between 1-7 additional variant amino acids.

In some embodiments, a VL comprising the amino acid sequence set forth in SEQ ID NO: 2 comprises an amino acid variant at positions 26, 27, 31, 56, 77, and 96 (IMGT positions 27, 28, 38, 70, 94, and 115). In some embodiments, a VL comprising the amino acid sequence set forth in SEQ ID NO: 2 comprises an amino acid variant at positions 26, 27, 31, 56, 77, 92, and 96 (IMGT positions 27, 28, 38, 70, 94, 109, and 115). In some embodiments, a VL comprising the amino acid sequence set forth in SEQ ID NO: 2 comprises an amino acid variant at positions 26, 31, 56, and 77 (IMGT positions 27, 38, 70, and 94). In some embodiments, a VL comprising the amino acid sequence set forth in SEQ ID NO: 2 comprises an amino acid variant at positions 26, 27, 31, 56, and 77 (IMGT positions 27, 28, 38, 70, and 94). In some embodiments, a VL comprising the amino acid sequence set forth in SEQ ID NO: 2 comprises an amino acid variant at positions 26, 27, 31, 56, 77, and 92 (IMGT positions 27, 28, 38, 70, 94, and 109). In some embodiments, a VL comprising the amino acid sequence set forth in SEQ ID NO: 2 comprises an amino acid variant at positions 26, 27, 31, 51, 56, 77, and 92 (IMGT positions 27, 28, 38, 65, 70, 94, and 109). In some embodiments, a VL comprising the amino acid sequence set forth in SEQ ID NO: 2 comprises an amino acid variant at positions 26, 31, 56, 77, 92, and 96 (IMGT positions 27, 38, 70, 94, 109, and 115). In some embodiments, a VL comprising the amino acid sequence set forth in SEQ ID NO: 2 comprises an amino acid variant at positions 26, 31, 77, and 92 (IMGT positions 27, 38, 94, and 109). In some embodiments, a VL comprising the amino acid sequence set forth in SEQ ID NO: 2 comprises an amino acid variant at positions 26, 27, 31, 56, 77, and 93 (IMGT positions 27, 28, 38, 70, 94, and 110). In some embodiments, a VL comprising the amino acid sequence set forth in SEQ ID NO: 2 comprises an amino acid variant at positions 26, 27, 31, 56, 77, 93, and 96 (IMGT positions 27, 28, 38, 70, 94, 110, and 115). In some embodiments, a VL comprising the amino acid sequence set forth in SEQ ID NO: 2 comprises an amino acid variant at positions 26, 27, 31, 51, 77, 93, and 96 (IMGT positions 27, 28, 38, 65, 94, 110, and 115). In some embodiments, a VL comprising the amino acid sequence set forth in SEQ ID NO: 2 comprises an amino acid variant at positions 26, 27, 31, 56, 77, 93, and 96 (IMGT positions 27, 28, 38, 70, 94, 110, and 115). In some embodiments, a VL comprising the amino acid sequence set forth in SEQ ID NO: 2 comprises an amino acid variant at positions 27, 31, 56, 77, and 96 (IMGT positions 28, 38, 70, 94, and 115). In some embodiments, a VL comprising the amino acid sequence set forth in SEQ ID NO: 2 comprises an amino acid variant at positions 27, 31, 56, 77, 92, and 96 (IMGT positions 28, 38, 70, 94, 109, and 115). In some embodiments, a VL comprising the amino acid sequence set forth in SEQ ID NO: 2 comprises an amino acid variant at positions 26, 27, 31, 51, 77, and 96 (IMGT positions 27, 28, 38, 65, 94, and 115). In some embodiments, a VL comprising the amino acid sequence set forth in SEQ ID NO: 2 comprises an amino acid variant at positions 26, 27, 31, or 96 (IMGT positions 27, 28, 38, and 115), or any combination thereof.

In some embodiments, a VL comprising the amino acid sequence set forth in SEQ ID NO: 2 comprises amino acid variants at positions in a framework region at any of positions 56 or 77 of SEQ ID NO: 2, or a combination thereof (IMGT positions: 70 or 94, or a combination thereof). In some embodiments, a VL comprising the amino acid sequence set forth in SEQ ID NO: 2 comprises at least one amino acid variant at a position within a framework region at any of positions 56 or 77 of SEQ ID NO: 2, or a combination thereof (IMGT positions: 70 or 94, or a combination thereof), wherein the variant amino acid at position 56 comprises a leucine, an alanine, an arginine, a lysine, an aspartic acid, a glycine, or a glutamic acid, and or the variant amino acid at position 77 comprises a valine.

In some embodiments, disclosed herein is an isolated dual binding antibody comprising: a heavy chain variable region comprising the amino acid sequence set forth in SEQ ID NO: 1 with at least one amino acid variant at any of positions (IMGT) 57, 107, 108, 109, 110, 111, 111A, 112A, 112, 113, 114, or 117, or a combination thereof; and a light chain variable region comprising the amino acid sequence set forth in SEQ ID NO: 2 with at least one amino acid variant at any of positions (IMGT) 27, 28, 38, 65, 70, 94, 109, 110, or 115, or a combination thereof; wherein the total number of variant positions in said dual binding antibody is at least 2.

In some embodiments, an isolated dual binding antibody comprises a variant VH or a variant VL, or a combination thereof, with variant amino acids at positions other than (IMGT) 57, 107, 108, 109, 110, 111, 111A, 112A, 112, 113, 114, or 117 in the heavy chain variable region and or (IMGT) 27, 28, 38, 65, 70, 94, 109, 110, or 115 in the light chain variable region.

In some embodiments, an at least one amino acid variant in a VH comprises a variant amino acid in a CDR region. In some embodiments, an at least one amino acid variant in a VH comprises a variant amino acid in a CDR1 region. In some embodiments, an at least one amino acid variant in a VH comprises a variant amino acid in a CDR2 region. In some embodiments, an at least one amino acid variant in a VH comprises a variant amino acid in a CDR3. In some embodiments, an at least two amino acid variants in a VH comprises variant amino acids in two different CDR regions. In some embodiments, an at least two amino acid variants in a VH comprises variant amino acids in a same CDR region. In some embodiments, an at least two amino acid variants in a VH comprises variant amino acids in a same CDR1 region. In some embodiments, an at least two amino acid variants in a VH comprises variant amino acids in a same CDR2 region. In some embodiments, an at least two amino acid variants in a VH comprises variant amino acids in a same CDR3 region. In some embodiments, an at least two amino acid variants in a VH comprises variant amino acids in a CDR1 and a CDR2 region. In some embodiments, an at least two amino acid variants in a VH comprises variant amino acids in a CDR1 and a CDR3 region. In some embodiments, an at least two amino acid variants in a VH comprises variant amino acids in a CDR2 and a CDR3 region. In some embodiments, an at least three amino acid variants in a VH comprises variant amino acids in a single CDR region. In some embodiments, an at least three amino acid variants in a VH comprises variant amino acids in a CDR1 region. In some embodiments, an at least three amino acid variants in a VH comprises variant amino acids in a CDR2 region. In some embodiments, an at least three amino acid variants in a VH comprises variant amino acids in a CDR3 region. In some embodiments, an at least three amino acid variants in a VH comprises variant amino acids in a CDR1 region and a CDR2 region. In some embodiments, an at least three amino acid variants in a VH comprises variant amino acids in a CDR1 region and a CDR3 region. In some embodiments, an at least three amino acid variants in a VH comprises variant amino acids in a CDR2 region and a CDR3 region. In some embodiments, an at least three amino acid variants in a VH comprises variant amino acids in a CDR1 region, a CDR2 region, and a CDR3 region. In some embodiments, an at least 4 amino acid variants in a VH comprises variant amino acids in a single CDR region. In some embodiments, an at least 4 amino acid variants in a VH comprises variant amino acids in a CDR1 region. In some embodiments, an at least 4 amino acid variants in a VH comprises variant amino acids in a CDR2 region. In some embodiments, an at least 4 amino acid variants in a VH comprises variant amino acids in a CDR3 region. In some embodiments, an at least 4 amino acid variants in a VH comprises variant amino acids in a CDR1 region and a CDR2 region. In some embodiments, an at least 4 amino acid variants in a VH comprises variant amino acids in a CDR1 region and a CDR3 region. In some embodiments, an at least 4 amino acid variants in a VH comprises variant amino acids in a CDR2 region and a CDR3 region. In some embodiments, an at least 4 amino acid variants in a VH comprises variant amino acids in a CDR1 region, a CDR2 region, and a CDR3 region. In some embodiments, when there are 5 or more amino acid variants in a VH, variant positions comprise variant amino acids in a single CDR region. In some embodiments, when there are 5 or more amino acid variants in a VH, variant positions comprise amino acids in a CDR1 region. In some embodiments, when there are 5 or more amino acid variants in a VH, variant positions comprise variant amino acids in a CDR2 region. In some embodiments, when there are 5 or more amino acid variants in a VH, variant positions comprise variant amino acids in a CDR3 region. In some embodiments, when there are 5 or more amino acid variants in a VH, variant positions comprise variant amino acids in a CDR1 region and a CDR2 region. In some embodiments, when there are 5 or more amino acid variants in a VH, variant positions comprise variant amino acids in a CDR1 region and a CDR3 region. In some embodiments, when there are 5 or more amino acid variants in a VH, variant positions comprise variant amino acids in a CDR2 region and a CDR3 region. In some embodiments, when there are 5 or more amino acid variants in a VH, variant positions comprise variant amino acids in a CDR1 region, a CDR2 region, and a CDR3 region.

In some embodiments, an at least one amino acid variant in a VL comprises a variant amino acid in a CDR region. In some embodiments, an at least one amino acid variant in a VL comprises a variant amino acid in a CDR1 region. In some embodiments, an at least one amino acid variant in a VL comprises a variant amino acid in a CDR2 region. In some embodiments, an at least one amino acid variant in a VL comprises a variant amino acid in a CDR3. In some embodiments, an at least two amino acid variants in a VL comprises variant amino acids in two different CDR regions. In some embodiments, an at least two amino acid variants in a VL comprises variant amino acids in a same CDR region. In some embodiments, an at least two amino acid variants in a VL comprises variant amino acids in a same CDR1 region. In some embodiments, an at least two amino acid variants in a VL comprises variant amino acids in a same CDR2 region. In some embodiments, an at least two amino acid variants in a VL comprises variant amino acids in a same CDR3 region. In some embodiments, an at least two amino acid variants in a VL comprises variant amino acids in a CDR1 and a CDR2 region. In some embodiments, an at least two amino acid variants in a VL comprises variant amino acids in a CDR1 and a CDR3 region. In some embodiments, an at least two amino acid variants in a VL comprises variant amino acids in a CDR2 and a CDR3 region. In some embodiments, an at least three amino acid variants in a VL comprises variant amino acids in a single CDR region. In some embodiments, an at least three amino acid variants in a VL comprises variant amino acids in a CDR1 region. In some embodiments, an at least three amino acid variants in a VL comprises variant amino acids in a CDR2 region. In some embodiments, an at least three amino acid variants in a VL comprises variant amino acids in a CDR3 region. In some embodiments, an at least three amino acid variants in a VL comprises variant amino acids in a CDR1 region and a CDR2 region. In some embodiments, an at least three amino acid variants in a VL comprises variant amino acids in a CDR1 region and a CDR3 region. In some embodiments, an at least three amino acid variants in a VL comprises variant amino acids in a CDR2 region and a CDR3 region. In some embodiments, an at least three amino acid variants in a VL comprises variant amino acids in a CDR1 region, a CDR2 region, and a CDR3 region. In some embodiments, an at least 4 amino acid variants in a VL comprises variant amino acids in a single CDR region. In some embodiments, an at least 4 amino acid variants in a VL comprises variant amino acids in a CDR1 region. In some embodiments, an at least 4 amino acid variants in a VL comprises variant amino acids in a CDR2 region. In some embodiments, an at least 4 amino acid variants in a VL comprises variant amino acids in a CDR3 region. In some embodiments, an at least 4 amino acid variants in a VL comprises variant amino acids in a CDR1 region and a CDR2 region. In some embodiments, an at least 4 amino acid variants in a VL comprises variant amino acids in a CDR1 region and a CDR3 region. In some embodiments, an at least 4 amino acid variants in a VL comprises variant amino acids in a CDR2 region and a CDR3 region. In some embodiments, an at least 4 amino acid variants in a VL comprises variant amino acids in a CDR1 region, a CDR2 region, and a CDR3 region. In some embodiments, when there are 5 amino acid variants in a VL, variant positions comprise variant amino acids in a single CDR region. In some embodiments, when there are 5 amino acid variants in a VL, variant positions comprise amino acids in a CDR1 region. In some embodiments, when there are 5 amino acid variants in a VL, variant positions comprise variant amino acids in a CDR2 region. In some embodiments, when there are 5 amino acid variants in a VL, variant positions comprise variant amino acids in a CDR3 region. In some embodiments, when there are 5 amino acid variants in a VL, variant positions comprise variant amino acids in a CDR1 region and a CDR2 region. In some embodiments, when there are 5 amino acid variants in a VL, variant positions comprise variant amino acids in a CDR1 region and a CDR3 region. In some embodiments, when there are 5 amino acid variants in a VL, variant positions comprise variant amino acids in a CDR2 region and a CDR3 region. In some embodiments, when there are 5 amino acid variants in a VL, variant positions comprise variant amino acids in a CDR1 region, a CDR2 region, and a CDR3 region. In some embodiments, when there are 6 amino acid variants in a VL, variant positions comprise variant amino acids in a single CDR region. In some embodiments, when there are 6 amino acid variants in a VL, variant positions comprise amino acids in a CDR1 region. In some embodiments, when there are 6 amino acid variants in a VL, variant positions comprise variant amino acids in a CDR2 region. In some embodiments, when there are 6 amino acid variants in a VL, variant positions comprise variant amino acids in a CDR3 region. In some embodiments, when there are 6 amino acid variants in a VL, variant positions comprise variant amino acids in a CDR1 region and a CDR2 region. In some embodiments, when there are 6 amino acid variants in a VL, variant positions comprise variant amino acids in a CDR1 region and a CDR3 region. In some embodiments, when there are 6 amino acid variants in a VL, variant positions comprise variant amino acids in a CDR2 region and a CDR3 region. In some embodiments, when there are 6 amino acid variants in a VL, variant positions comprise variant amino acids in a CDR1 region, a CDR2 region, and a CDR3 region. In some embodiments, when there are 7 or more amino acid variants in a VL, variant positions comprise variant amino acids in a single CDR region. In some embodiments, when there are 7 or more amino acid variants in a VL, variant positions comprise amino acids in a CDR1 region. In some embodiments, when there are 7 or more amino acid variants in a VL, variant positions comprise variant amino acids in a CDR2 region. In some embodiments, when there are 7 or more amino acid variants in a VL, variant positions comprise variant amino acids in a CDR3 region. In some embodiments, when there are 7 or more amino acid variants in a VL, variant positions comprise variant amino acids in a CDR1 region and a CDR2 region. In some embodiments, when there are 7 or more amino acid variants in a VL, variant positions comprise variant amino acids in a CDR1 region and a CDR3 region. In some embodiments, when there are 7 or more amino acid variants in a VL, variant positions comprise variant amino acids in a CDR2 region and a CDR3 region. In some embodiments, when there are 7 or more amino acid variants in a VL, variant positions comprise variant amino acids in a CDR1 region, a CDR2 region, and a CDR3 region.

In some embodiments, an amino acid variant comprises a substitution of one amino acid residue for another. In some embodiments, an amino acid variant comprises a substitution of a hydrophobic residue for a non-hydrophobic residue. In some embodiments, an amino acid variant comprises a substitution of a charged residue for a non-charged residue. In some embodiments, an amino acid variant comprises a neutral substitution, wherein the amino acid being substituted has similar qualities. In some embodiments, an amino acid variant comprises a substitution of an aromatic residue for a non-aromatic residue. In some embodiments, natural aromatic amino acids such as Trp, Tyr and Phe, are substituted for synthetic non-natural acid such as Phenylglycine, TIC, naphthylelanine (Nol), ring-methylated derivatives of Phe, halogenated derivatives of Phe or o-methyl-Tyr. In some embodiments, a variant substitution comprises substituting a modified amino acid or a non-amino acid monomer (e.g. fatty acid, complex carbohydrates etc.). A skilled artisan would appreciate that while the choice of amino acid residues at each variant position may in certain embodiments, affect the 3D structure of the VH, VL, and/or combination thereof, the choice of amino acid residues at each variant position is considered independently.

In some embodiments, “amino acid” or “amino acid residue” or “residue” is understood to include the 20 naturally occurring, encoded amino acid residues, and those amino acids often modified post-translationally in vivo, including, for example, hydroxyproline, phosphoserine and phosphothreonine; and other unusual amino acid including, but not limited to, 2-aminoadipic acid, hydroxylysine, isodesmosine, nor-valine, nor-leucine and ornithine. In some embodiments, “amino acid” includes both D- and L-amino acid. In some embodiments, an amino acid variant substitution is a D-amino acid. In some embodiments, an amino acid variant substitution is an L-amino acid. In some embodiments, a variant residue comprises a naturally occurring amino acid. In some embodiments, a variant residue comprises a naturally occurring, encoded amino acid residue. In some embodiments, a variant residue comprises a naturally occurring, non-encoded amino acid residue. In some embodiments, a variant residue comprises a non-naturally occurring amino acid.

In some embodiments, a variant residue comprises a non-naturally occurring, non-proteinogenic amino acid.

In some embodiments, the amino acid sequence of a VH domain of the dual binding antibody is selected from, but not limited to, the sequences set forth in any of SEQ ID NOs: 4, 6, 8, 10, 12, 14, 16, 18, 20, 22, 24, 26, 28, 30, 32, 34, 36, 38, 40, 42, 44, 46, 48, 50, 52, and 54. In some embodiments, an isolated dual binding antibody comprises a heavy chain variable region comprising the amino acid sequences set forth in any of SEQ ID NOs: 4, 6, 8, 10, 12, 14, 16, 18, 20, 22, 24, 26, 28, 30, 32, 34, 36, 38, 40, 42, 44, 46, 48, 50, 52, and 54; and any variable light chain region. In some embodiments, an isolated dual binding antibody comprises a heavy chain variable region comprising the amino acid sequences set forth in SEQ ID NOs: 4 and any variable light chain region. In some embodiments, an isolated dual binding antibody comprises a heavy chain variable region comprising the amino acid sequences set forth in SEQ ID NOs: 6 and any variable light chain region. In some embodiments, an isolated dual binding antibody comprises a heavy chain variable region comprising the amino acid sequences set forth in SEQ ID NOs: 8 and any variable light chain region. In some embodiments, an isolated dual binding antibody comprises a heavy chain variable region comprising the amino acid sequences set forth in SEQ ID NOs: 10 and any variable light chain region. In some embodiments, an isolated dual binding antibody comprises a heavy chain variable region comprising the amino acid sequences set forth in SEQ ID NOs: 12 and any variable light chain region. In some embodiments, an isolated dual binding antibody comprises a heavy chain variable region comprising the amino acid sequences set forth in SEQ ID NOs: 14 and any variable light chain region. In some embodiments, an isolated dual binding antibody comprises a heavy chain variable region comprising the amino acid sequences set forth in SEQ ID NOs: 16 and any variable light chain region. In some embodiments, an isolated dual binding antibody comprises a heavy chain variable region comprising the amino acid sequences set forth in SEQ ID NOs:18 and any variable light chain region. In some embodiments, an isolated dual binding antibody comprises a heavy chain variable region comprising the amino acid sequences set forth in SEQ ID NOs: 20 and any variable light chain region. In some embodiments, an isolated dual binding antibody comprises a heavy chain variable region comprising the amino acid sequences set forth in SEQ ID NOs: 22 and any variable light chain region. In some embodiments, an isolated dual binding antibody comprises a heavy chain variable region comprising the amino acid sequences set forth in SEQ ID NOs: 24 and any variable light chain region. In some embodiments, an isolated dual binding antibody comprises a heavy chain variable region comprising the amino acid sequences set forth in SEQ ID NOs: 26 and any variable light chain region. In some embodiments, an isolated dual binding antibody comprises a heavy chain variable region comprising the amino acid sequences set forth in SEQ ID NOs: 28 and any variable light chain region. In some embodiments, an isolated dual binding antibody comprises a heavy chain variable region comprising the amino acid sequences set forth in SEQ ID NOs: 30 and any variable light chain region. In some embodiments, an isolated dual binding antibody comprises a heavy chain variable region comprising the amino acid sequences set forth in SEQ ID NOs: 32 and any variable light chain region. In some embodiments, an isolated dual binding antibody comprises a heavy chain variable region comprising the amino acid sequences set forth in SEQ ID NOs: 34 and any variable light chain region. In some embodiments, an isolated dual binding antibody comprises a heavy chain variable region comprising the amino acid sequences set forth in SEQ ID NOs: 36 and any variable light chain region. In some embodiments, an isolated dual binding antibody comprises a heavy chain variable region comprising the amino acid sequences set forth in SEQ ID NOs: 38 and any variable light chain region. In some embodiments, an isolated dual binding antibody comprises a heavy chain variable region comprising the amino acid sequences set forth in SEQ ID NOs: 40 and any variable light chain region. In some embodiments, an isolated dual binding antibody comprises a heavy chain variable region comprising the amino acid sequences set forth in SEQ ID NOs: 42 and any variable light chain region. In some embodiments, an isolated dual binding antibody comprises a heavy chain variable region comprising the amino acid sequences set forth in SEQ ID NOs: 44 and any variable light chain region. In some embodiments, an isolated dual binding antibody comprises a heavy chain variable region comprising the amino acid sequences set forth in SEQ ID NOs: 46 and any variable light chain region. In some embodiments, an isolated dual binding antibody comprises a heavy chain variable region comprising the amino acid sequences set forth in SEQ ID NOs: 48 and any variable light chain region. In some embodiments, an isolated dual binding antibody comprises a heavy chain variable region comprising the amino acid sequences set forth in SEQ ID NOs: 50 and any variable light chain region. In some embodiments, an isolated dual binding antibody comprises a heavy chain variable region comprising the amino acid sequences set forth in SEQ ID NOs: 52 and any variable light chain region. In some embodiments, an isolated dual binding antibody comprises a heavy chain variable region comprising the amino acid sequences set forth in SEQ ID NOs: 54 and any variable light chain region.

In some embodiments, the amino acid sequence of a VH domain of the dual binding antibody is one of those set forth in Table 1 or Table 10. In some embodiments, an isolated dual binding antibody comprises a heavy chain variable region comprising one of the amino acid sequences set forth in Table 1 or Table 10; and any variable light chain region.

In some embodiments, the amino acid sequence of a VL domain of the dual binding antibody is selected from, but not limited to, the sequences set forth in any of SEQ ID NOs: 3, 5, 7, 9, 11, 13, 15, 17, 19 21, 23, 25, 27, 29, 31, 33, 35, 37, 39, 41, 43, 45, 47, 49, 51, and 53. In some embodiments, an isolated dual binding antibody comprises a light chain variable region comprising the amino acid sequences set forth in any of SEQ ID NOs: 3, 5, 7, 9, 11, 13, 15, 17, 19 21, 23, 25, 27, 29, 31, 33, 35, 37, 39, 41, 43, 45, 47, 49, 51, and 53; and any variable heavy chain region. In some embodiments, an isolated dual binding antibody comprises a light chain variable region comprising the amino acid sequences set forth in SEQ ID NOs: 3 and any variable heavy chain region. In some embodiments, an isolated dual binding antibody comprises a light chain variable region comprising the amino acid sequences set forth in SEQ ID NOs: 5 and any variable heavy chain region. In some embodiments, an isolated dual binding antibody comprises a light chain variable region comprising the amino acid sequences set forth in SEQ ID NOs: 7 and any variable heavy chain region. In some embodiments, an isolated dual binding antibody comprises a light chain variable region comprising the amino acid sequences set forth in SEQ ID NOs: 9 and any variable heavy chain region. In some embodiments, an isolated dual binding antibody comprises a light chain variable region comprising the amino acid sequences set forth in SEQ ID NOs: 11 and any variable heavy chain region. In some embodiments, an isolated dual binding antibody comprises a light chain variable region comprising the amino acid sequences set forth in SEQ ID NOs: 13 and any variable heavy chain region. In some embodiments, an isolated dual binding antibody comprises a light chain variable region comprising the amino acid sequences set forth in SEQ ID NOs: 15 and any variable heavy chain region. In some embodiments, an isolated dual binding antibody comprises a light chain variable region comprising the amino acid sequences set forth in SEQ ID NOs: 17 and any variable heavy chain region. In some embodiments, an isolated dual binding antibody comprises a light chain variable region comprising the amino acid sequences set forth in SEQ ID NOs: 19 and any variable heavy chain region. In some embodiments, an isolated dual binding antibody comprises a light chain variable region comprising the amino acid sequences set forth in SEQ ID NOs: 21 and any variable heavy chain region. In some embodiments, an isolated dual binding antibody comprises a light chain variable region comprising the amino acid sequences set forth in SEQ ID NOs: 23 and any variable heavy chain region. In some embodiments, an isolated dual binding antibody comprises a light chain variable region comprising the amino acid sequences set forth in SEQ ID NOs: 25 and any variable heavy chain region. In some embodiments, an isolated dual binding antibody comprises a light chain variable region comprising the amino acid sequences set forth in SEQ ID NOs: 27 and any variable heavy chain region. In some embodiments, an isolated dual binding antibody comprises a light chain variable region comprising the amino acid sequences set forth in SEQ ID NOs: 29 and any variable heavy chain region. In some embodiments, an isolated dual binding antibody comprises a light chain variable region comprising the amino acid sequences set forth in SEQ ID NOs: 31 and any variable heavy chain region. In some embodiments, an isolated dual binding antibody comprises a light chain variable region comprising the amino acid sequences set forth in SEQ ID NOs: 33 and any variable heavy chain region. In some embodiments, an isolated dual binding antibody comprises a light chain variable region comprising the amino acid sequences set forth in SEQ ID NOs: 35 and any variable heavy chain region. In some embodiments, an isolated dual binding antibody comprises a light chain variable region comprising the amino acid sequences set forth in SEQ ID NOs: 37 and any variable heavy chain region. In some embodiments, an isolated dual binding antibody comprises a light chain variable region comprising the amino acid sequences set forth in SEQ ID NOs: 39 and any variable heavy chain region. In some embodiments, an isolated dual binding antibody comprises a light chain variable region comprising the amino acid sequences set forth in SEQ ID NOs: 42 and any variable heavy chain region. In some embodiments, an isolated dual binding antibody comprises a light chain variable region comprising the amino acid sequences set forth in SEQ ID NOs: 43 and any variable heavy chain region. In some embodiments, an isolated dual binding antibody comprises a light chain variable region comprising the amino acid sequences set forth in SEQ ID NOs: 45 and any variable heavy chain region. In some embodiments, an isolated dual binding antibody comprises a light chain variable region comprising the amino acid sequences set forth in SEQ ID NOs: 47 and any variable heavy chain region. In some embodiments, an isolated dual binding antibody comprises a light chain variable region comprising the amino acid sequences set forth in SEQ ID NOs: 49 and any variable heavy chain region. In some embodiments, an isolated dual binding antibody comprises a light chain variable region comprising the amino acid sequences set forth in SEQ ID NOs: 51 and any variable heavy chain region. In some embodiments, an isolated dual binding antibody comprises a light chain variable region comprising the amino acid sequences set forth in SEQ ID NOs: 53 and any variable heavy chain region.

In some embodiments, the amino acid sequence of a VL domain of the dual binding antibody is one of those set forth Table 1 or Table 10. In some embodiments, an isolated dual binding antibody comprises a light chain variable region comprising one of the amino acid sequences set forth in Table 1 or Table 10; and any variable heavy chain region.

A skilled artisan would recognize that when pairing a VH or VL domain comprising a known amino acid sequence, with a VL or VH domain, respectively, to comprise an antigen binding region, such a pairing may be analyzed for binding properties using methods well known in the art (See for example, the disclosure herein and Examples below).

In some embodiments, the amino acid sequence of a heavy chain variable region—light chain variable region pair are selected from, but not limited to, the pair sequences set forth in SEQ ID NOs: 4 and 3, SEQ ID Nos: 6 and 5, SEQ ID Nos: 8 and 7, SEQ ID Nos: 10 and 9, SEQ ID Nos: 12 and 11, SEQ ID Nos: 14 and 13, SEQ ID Nos: 16 and 15, SEQ ID Nos: 18 and 17, SEQ ID Nos: 20 and 19, SEQ ID Nos: 22 and 21, SEQ ID Nos: 24 and 23, SEQ ID Nos: 26 and 25, SEQ ID Nos: 28 and 27, SEQ ID Nos: 30 and 29, SEQ ID Nos: 32 and 31, SEQ ID Nos: 34 and 33, SEQ ID Nos: 36 and 35, SEQ ID Nos: 38 and 37, SEQ ID Nos: 40 and 39, SEQ ID Nos: 42 and 41, SEQ ID Nos: 44 and 43, SEQ ID Nos: 46 and 45, SEQ ID Nos: 48 and 47, SEQ ID Nos: 50 and 49, SEQ ID Nos: 52 and 51, and SEQ ID Nos: 54 and 53. In some embodiments, an isolated dual binding antibody comprises a heavy chain variable region-light chain variable region pair are selected from the pair sequences set forth in SEQ ID NOs: 4 and 3. In some embodiments, an isolated dual binding antibody comprises a heavy chain variable region-light chain variable region pair are selected from the pair sequences set forth in SEQ ID NOs: SEQ ID Nos: 6 and 5. In some embodiments, an isolated dual binding antibody comprises a heavy chain variable region-light chain variable region pair are selected from the pair sequences set forth in SEQ ID NOs: SEQ ID Nos: 8 and 7. In some embodiments, an isolated dual binding antibody comprises a heavy chain variable region-light chain variable region pair are selected from the pair sequences set forth in SEQ ID NOs: SEQ ID Nos: 10 and 9. In some embodiments, an isolated dual binding antibody comprises a heavy chain variable region-light chain variable region pair are selected from the pair sequences set forth in SEQ ID NOs: SEQ ID Nos: 12 and 11. In some embodiments, an isolated dual binding antibody comprises a heavy chain variable region-light chain variable region pair are selected from the pair sequences set forth in SEQ ID NOs: SEQ ID Nos: 14 and 13. In some embodiments, an isolated dual binding antibody comprises a heavy chain variable region-light chain variable region pair are selected from the pair sequences set forth in SEQ ID NOs: SEQ ID Nos: 16 and 15. In some embodiments, an isolated dual binding antibody comprises a heavy chain variable region-light chain variable region pair are selected from the pair sequences set forth in SEQ ID NOs: SEQ ID Nos: 18 and 17. In some embodiments, an isolated dual binding antibody comprises a heavy chain variable region-light chain variable region pair are selected from the pair sequences set forth in SEQ ID NOs: SEQ ID Nos: 20 and 19. In some embodiments, an isolated dual binding antibody comprises a heavy chain variable region-light chain variable region pair are selected from the pair sequences set forth in SEQ ID NOs: SEQ ID Nos: 22 and 21. In some embodiments, an isolated dual binding antibody comprises a heavy chain variable region-light chain variable region pair are selected from the pair sequences set forth in SEQ ID NOs: SEQ ID Nos: 24 and 23. In some embodiments, an isolated dual binding antibody comprises a heavy chain variable region-light chain variable region pair are selected from the pair sequences set forth in SEQ ID NOs: SEQ ID Nos: 26 and 25. In some embodiments, an isolated dual binding antibody comprises a heavy chain variable region-light chain variable region pair are selected from the pair sequences set forth in SEQ ID NOs: SEQ ID Nos: 28 and 27. In some embodiments, an isolated dual binding antibody comprises a heavy chain variable region-light chain variable region pair are selected from the pair sequences set forth in SEQ ID NOs: SEQ ID Nos: 30 and 29. In some embodiments, an isolated dual binding antibody comprises a heavy chain variable region-light chain variable region pair are selected from the pair sequences set forth in SEQ ID NOs: SEQ ID Nos: 32 and 31. In some embodiments, an isolated dual binding antibody comprises a heavy chain variable region-light chain variable region pair are selected from the pair sequences set forth in SEQ ID NOs: SEQ ID Nos: 34 and 33. In some embodiments, an isolated dual binding antibody comprises a heavy chain variable region-light chain variable region pair are selected from the pair sequences set forth in SEQ ID NOs: SEQ ID Nos: 36 and 35. In some embodiments, an isolated dual binding antibody comprises a heavy chain variable region-light chain variable region pair are selected from the pair sequences set forth in SEQ ID NOs: SEQ ID Nos: 38 and 37. In some embodiments, an isolated dual binding antibody comprises a heavy chain variable region-light chain variable region pair are selected from the pair sequences set forth in SEQ ID NOs: SEQ ID Nos: 40 and 39. In some embodiments, an isolated dual binding antibody comprises a heavy chain variable region-light chain variable region pair are selected from the pair sequences set forth in SEQ ID NOs: SEQ ID Nos: 42 and 41. In some embodiments, an isolated dual binding antibody comprises a heavy chain variable region-light chain variable region pair are selected from the pair sequences set forth in SEQ ID NOs: SEQ ID Nos: 44 and 43. In some embodiments, an isolated dual binding antibody comprises a heavy chain variable region-light chain variable region pair are selected from the pair sequences set forth in SEQ ID NOs: SEQ ID Nos: 46 and 45. In some embodiments, an isolated dual binding antibody comprises a heavy chain variable region-light chain variable region pair are selected from the pair sequences set forth in SEQ ID NOs: 48 and 47. In some embodiments, an isolated dual binding antibody comprises a heavy chain variable region-light chain variable region pair are selected from the pair sequences set forth in SEQ ID NOs: 50 and 49. In some embodiments, an isolated dual binding antibody comprises a heavy chain variable region-light chain variable region pair are selected from the pair sequences set forth in SEQ ID NOs: 52 and 51. In some embodiments, an isolated dual binding antibody comprises a heavy chain variable region-light chain variable region pair are selected from the pair sequences set forth in SEQ ID Nos: 54 and 53.

In some embodiments, the amino acid sequences of a heavy chain variable region-light chain variable region pair are selected from the pair sequences set forth in any one of the following: SEQ ID NOs:209 and 210, SEQ ID NOs:211 and 212, SEQ ID NOs:213 and 214, SEQ ID NOs:215 and 216, SEQ ID NOs:217 and 218, SEQ ID NOs:219 and 220, SEQ ID NOs:221 and 222, SEQ ID NOs:223 and 224, SEQ ID NOs:225 and 226, SEQ ID NOs:227 and 228, SEQ ID NOs:229 and 230, SEQ ID NOs:231 and 232, SEQ ID NOs:233 and 234, SEQ ID NOs:235 and 236, SEQ ID NOs:237 and 238, SEQ ID NOs:239 and 240, SEQ ID NOs:241 and 242, SEQ ID NOs:243 and 244, SEQ ID NOs:245 and 246, SEQ ID NOs:247 and 248, SEQ ID NOs:249 and 250, SEQ ID NOs:251 and 252, SEQ ID NOs:253 and 254, SEQ ID NOs:255 and 256, SEQ ID NOs:257 and 258, SEQ ID NOs:259 and 260, SEQ ID NOs:261 and 262, SEQ ID NOs:263 and 264, SEQ ID NOs:265 and 266, SEQ ID NOs:267 and 268, SEQ ID NOs:269 and 270, SEQ ID NOs:271 and 272, SEQ ID NOs:273 and 274, SEQ ID NOs:275 and 276, SEQ ID NOs:277 and 278, SEQ ID NOs:279 and 280, SEQ ID NOs:281 and 282, SEQ ID NOs:283 and 284, SEQ ID NOs:285 and 286, SEQ ID NOs:287 and 288, SEQ ID NOs:289 and 290, SEQ ID NOs:291 and 292, SEQ ID NOs:293 and 294, SEQ ID NOs:295 and 296, SEQ ID NOs:297 and 298, SEQ ID NOs:299 and 300, SEQ ID NOs:301 and 302, SEQ ID NOs:303 and 304, SEQ ID NOs:305 and 306, SEQ ID NOs:307 and 308, SEQ ID NOs:309 and 310, SEQ ID NOs:311 and 312, SEQ ID NOs:313 and 314, SEQ ID NOs:315 and 316, SEQ ID NOs:317 and 318, SEQ ID NOs:319 and 320, SEQ ID NOs:321 and 322, SEQ ID NOs:323 and 324, SEQ ID NOs:325 and 326, SEQ ID NOs:327 and 328, SEQ ID NOs:329 and 330, SEQ ID NOs:331 and 332, SEQ ID NOs:333 and 334, SEQ ID NOs:335 and 336, SEQ ID NOs:337 and 338, SEQ ID NOs:339 and 340, SEQ ID NOs:341 and 342, SEQ ID NOs:343 and 344, SEQ ID NOs:345 and 346, SEQ ID NOs:347 and 348.

In some embodiments, the amino acid sequence of an scFv fragment comprises the pair of sequences set forth in, but not limited to, any of the following pairs: SEQ ID NOs: 4 and 3, SEQ ID Nos: 6 and 5, SEQ ID Nos: 8 and 7, SEQ ID Nos: 10 and 9, SEQ ID Nos: 12 and 11, SEQ ID Nos: 14 and 13, SEQ ID Nos: 16 and 15, SEQ ID Nos: 18 and 17, SEQ ID Nos: 20 and 19, SEQ ID Nos: 22 and 21, SEQ ID Nos: 24 and 23, SEQ ID Nos: 26 and 25, SEQ ID Nos: 28 and 27, SEQ ID Nos: 30 and 29, SEQ ID Nos: 32 and 31, SEQ ID Nos: 34 and 33, SEQ ID Nos: 36 and 35, SEQ ID Nos: 38 and 37, SEQ ID Nos: 40 and 39, SEQ ID Nos: 42 and 41, SEQ ID Nos: 44 and 43, SEQ ID Nos: 46 and 45, SEQ ID Nos: 48 and 47, SEQ ID Nos: 50 and 49, SEQ ID Nos: 52 and 51, and SEQ ID Nos: 54 and 53.

Nucleotide Sequences Encoding Engineered “Re-Epitoped” VH Domains, VL Domains, or Both VH and VL Domains, and Vectors and Host Cells Comprising these Nucleotide Sequences

The present disclosure provides dual binding antibodies comprising a VH domain, a VL domain, or both a VH and VL domain, comprising variant amino acid sequences compared with template VH and VL sequences SEQ ID NO: 1 and SEQ ID NO: 2, respectively. As described in detail above, in some embodiments the dual binding antibody comprises a heavy chain variable region comprising the amino acid sequence set forth in SEQ ID NO: 1 with at least one amino acid variant at any of positions 52, 99, 100, 101, 102, 103, 104, 105, 106, 107, 108, or 111, or any combination thereof (IMGT positions: 57, 107, 108, 109, 110, 111, 111A, 112A, 112, 113, 114, or 117, or a combination thereof); a light chain variable region comprising the amino acid sequence set forth in SEQ ID NO: 2 with at least one amino acid variant at any of positions 26, 27, 31, 51, 56, 77, 92, 93, or 96, or any combination thereof (IMGT positions: 27, 28, 38, 65, 70, 94, 109, 110, or 115, or a combination thereof); or a combination of heavy chain variable region set forth in (a) and the light chain variable region set forth in (b); wherein the total number of variant positions in the encoded heavy chain variable region, the encoded light chain variable region, or a combination thereof, is at least 2.

In some embodiments, a nucleic acid construct comprising a nucleic acid sequence encodes an isolated dual binding antibody comprises an antibody antigen-binding domain site comprising a VH domain and a VL domain, wherein said VH domain comprises a set of CDRs, HCDR1, HCDR2, and HCDR3 as disclosed herein. In some embodiments, a nucleic acid construct comprising a nucleic acid sequence encodes an isolated dual binding antibody comprises an antibody antigen-binding domain site comprising a VH domain and a VL domain, wherein said VH domain comprises a set of CDRs, HCDR1, HCDR2, and HCDR3 as disclosed in Table 8 or Table 4. In some embodiments, the amino acid sequence of HCDR1 is set forth in SEQ ID NO: 136; wherein the amino acid sequence of HCDR2 is set forth as: I HX1 Y D G S N K (SEQ ID NO: 142), wherein HX1 is any amino acid; and wherein the amino acid sequence of HCDR3 is set forth as: A R HX2 HX3 HX4 HX5 HX6 HX7 HX8 HX9 HX10 HX11 F D HX12 (SEQ ID NO: 143), wherein XH2, HX3, HX4, HX5, HX6, HX7, HX8, HX9, HX10, HX11, and HX12 are any amino acid.

In some embodiments, a nucleic acid construct comprising a nucleic acid sequence encodes a VH domain of a dual binding antibody comprises HCDR1 (SEQ ID NO: 136), HCDR2 (SEQ ID NO: 142), and HCDR3 (SEQ ID NO: 143), wherein the VH domain comprises a variant amino acid at, at least one of HX1, HX2, HX3, HX4, HX5, HX6, HX7, HX8, HX9, HX10, HX11, and HX12.

In some embodiments, a nucleic acid construct comprising a nucleic acid sequence encodes a VH domain of a dual binding antibody comprises HCDR1 (SEQ ID NO: 136), HCDR2 (SEQ ID NO: 137) wherein HX1 is selected from the group consisting of W and S; and HCDR3 (SEQ ID NO: 138) wherein HX2 is selected from the group consisting of A and S, wherein HX3 is P, wherein HX4 is Q, wherein HX5 is W, wherein HX6 is selected from the group consisting of E, Q, M, L, and V, wherein HX7 is selected from the group consisting of L, W, and Y, wherein HX8 is selected from the group consisting of V and T, wherein HX9 is selected from the group consisting of H, A, S, wherein HX10 Is E, wherein HX11 is A, wherein HX12 is selected from the group consisting of I, L, and M. In certain embodiments, a nucleic acid construct comprising a nucleic acid sequence encoding a dual binding antibody comprising variant amino acids comprising HCDR1 (SEQ ID NO: 136), HCDR2 (SEQ ID NO: 137) wherein HX1 is W, HX2 is selected from the group consisting of A and S, HX3 is P, HX4 is Q, HX5 is W, HX6 is selected from the group consisting of E and M, HX7 is selected from the group consisting of L and W, HX8 is selected from the group consisting of V and T, HX9 is selected from the group consisting of H and A, HX10 is E, HX11 is A, and HX12 is selected from the group I and L.

In some embodiments, a nucleic acid construct comprising a nucleic acid sequence encodes a dual binding antibody comprising a VH domain comprising an HCDR1 (SEQ ID NO: 136), an HCDR2 (SEQ ID NO: 137) wherein HX1 is W, and an HCDR3 (SEQ ID NO: 138) wherein HX2 is A, HX3 is P, HX4 is Q, HX5 is W, HX6 is E, HX7 is L, HX8 is T, HX9 is A, HX10 is E, HX11 is A, and HX12 is I; or an HCDR1 (SEQ ID NO: 136), an HCDR2 (SEQ ID NO: 137) wherein HX1 is W, and an HCDR3 (SEQ ID NO: 138) wherein HX2 is A, HX3 is P, HX4 is Q, HX5 is W, HX6 is M, HX7 is L, HX8 is V, HX9 is A, HX10 is E, HX11 is A, and HX12 is L; or an HCDR1 (SEQ ID NO: 136), an HCDR2 (SEQ ID NO: 137) wherein HX1 is W, and an HCDR3 (SEQ ID NO: 138) wherein HX2 is S, HX3 is P, HX4 is Q, HX5 is W, HX6 is E, HX7 is W, HX8 is V, HX9 is H, HX10 is E, HX11 is A, and HX12 is L.

In some embodiments, a nucleic acid construct comprising a nucleic acid sequence encodes an isolated dual binding antibody comprising an antibody antigen-binding domain site comprising a VH domain and a VL domain, wherein said in some embodiments the VL domain comprises a set of CDRs, LCDR1, LCDR2, and LCDR3, wherein the amino acid sequence of LCDR1 is set forth as LX1, LX2, G S K LX3 V (SEQ ID NO: 144), wherein LX1, LX2, and LX3 are any amino acid; wherein the amino acid sequence of LCDR2 is set forth as D D LX4, wherein LX4 is any amino acid; and wherein the amino acid sequence of LCDR3 is set forth as Q V W D LX5 LX6 S D LX7 V V (SEQ ID NO; 146), wherein LX5, LX6, and LX7 are any amino acid.

In some embodiments, a nucleic acid construct comprising a nucleic acid sequence encodes a VL domain of a dual binding antibody comprising LCDRs. In some embodiments, a nucleic acid construct comprising a nucleic acid sequence encodes an isolated dual binding antibody comprises an antibody antigen-binding domain site comprising a VH domain and a VL domain, wherein said VL domain comprises a set of CDRs, LCDR1, LCDR2, and LCDR3 as disclosed in Table 9 or Table 5. In some embodiments, the amino acid sequence of LCDR1 is set forth in SEQ ID NO: 139, wherein LX1 is selected from the group consisting of N, L, and I, wherein LX2 is selected from the group consisting of L and I, wherein LX3 is selected from the group consisting of S and L; wherein the amino acid sequence of LCDR2 is set forth as D D LX4, wherein LX4 is selected from the group consisting of S and G; and wherein the amino acid sequence of LCDR3 is set forth in SEQ ID NO: 141, wherein LX5 is selected from the group consisting of S and T, wherein LX6 is selected from the group consisting of S and G, and wherein LX7 is selected from the group consisting of H and G. In certain embodiments, a nucleic acid construct comprising a nucleic acid sequence encodes the isolated dual binding antibody comprising variant amino acids wherein LCDR1 (SEQ ID NO: 139) LX1 is L, LX2 is I, LX3 is L, LCDR2 (D D LX4, wherein LX4 is selected from the group consisting of S and G), LCDR3 (SEQ ID NO: 141) wherein LX5 is S, LX6 is S, and LX7 is selected from the group consisting of H and G.

In some embodiments, a nucleic acid construct comprising a nucleic acid sequence encodes a dual binding antibody comprising a VL domain comprising an LCDR1 (SEQ ID NO: 139) wherein LX1 is L, LX2 is I, and LX3 is L, an LCDR2 (D D LX4, wherein LX4 is S), and LCDR3 (SEQ ID NO: 141) wherein LX5 is S, LX6 is S, and LX7 is G; or an LCDR1 (SEQ ID NO: 139) wherein LX1 is L, LX2 is I, and LX3 is L, an LCDR2 (D D LX4, wherein LX4 is S), and LCDR3 (SEQ ID NO: 141) wherein LX5 is S, LX6 is S, and LX7 is H, or an LCDR1 (SEQ ID NO: 139) wherein LX1 is L, LX2 is I, and LX3 is L, an LCDR2 (D D LX4, wherein LX4 is G), and LCDR3 (SEQ ID NO: 141) wherein LX5 is S, LX6 is S, and LX7 is G.

In some embodiments, a nucleic acid construct comprising a nucleic acid sequence encodes an isolated dual binding antibody comprising an antibody antigen-binding domain site comprising a VH domain and a VL domain comprises a combination of VH domain HCDRs and VL domain LCDRs described above. For example, but not limited to, in certain embodiments, a nucleic acid construct comprising a nucleic acid sequence encodes a VH domain comprising a set of CDRs, HCDR1, HCDR2, and HCDR3, wherein the amino acid sequence of HCDR1 is set forth in SEQ ID NO: 136; wherein the amino acid sequence of HCDR2 is set forth as: I HX1 Y D G S N K (SEQ ID NO: 142), wherein HX1 is any amino acid; and wherein the amino acid sequence of HCDR3 is set forth as: A R HX2 HX3 HX4 HX5 HX6 HX7 HX8 HX9 HX10 HX11 F D HX12 (SEQ ID NO: 143), wherein XH2, HX3, HX4, HX5, HX6, HX7, HX8, HX9, HX10, HX11, and HX12 are any amino acid; and wherein said VL domain comprises a set of CDRs, LCDR1, LCDR2, and LCDR3, wherein the amino acid sequence of LCDR1 is set forth as LX1, LX2, G S K LX3 V (SEQ ID NO: 144), wherein LX1, LX2, and LX3 are any amino acid; wherein the amino acid sequence of LCDR2 is set forth as D D LX4, wherein LX4 is any amino acid; and wherein the amino acid sequence of LCD3 is set forth as Q V W D LX5 LX6 S D LX7 V V (SEQ ID NO; 146), wherein LX5, LX6, and LX7 are any amino acid.

In some embodiments, a nucleic acid construct comprising a nucleic acid sequence encodes a VH domain comprising a set of CDRs, HCDR1, HCDR2, and HCDR3, wherein the amino acid sequence of HCDR1 is set forth in SEQ ID NO: 136, wherein the amino acid sequence of HCDR2 is set forth in SEQ ID NO: 137, wherein HX1 is selected from the group consisting of W and S; wherein the amino acid sequence of HCDR3 is set forth in SEQ ID NO: 138, wherein HX2 is selected from the group consisting of A and S, wherein HX3 is P, wherein HX4 is Q, wherein HX5 is W, wherein HX6 is selected from the group consisting of E, Q, M, L, and V, wherein HX7 is selected from the group consisting of L, W, and Y, wherein HX8 is selected from the group consisting of V and T, wherein HX9 is selected from the group consisting of H, A, S, wherein HX10 is E, wherein HX11 is A, wherein HX12 is selected from the group consisting of I, L, and M; and a VL domain comprises a set of CDRs, HCDR1, HCDR2, and HCDR3, wherein the amino acid sequence of LCDR1 is set forth in SEQ ID NO: 139, wherein LX1 is selected from the group consisting of N, L, and I, wherein LX2 is selected from the group consisting of L and I, wherein LX3 is selected from the group consisting of S and L; wherein the amino acid sequence of LCDR2 is set forth as A D D LX4, wherein LX4 is selected from the group consisting of S and G; wherein the amino acid sequence of LCDR3 is set forth in SEQ ID NO: 141, wherein LX5 is selected from the group consisting of S and T, wherein LX6 is selected from the group consisting of S and G, and wherein LX7 is selected from the group consisting of H and G.

In certain embodiments, a nucleic acid construct comprising a nucleic acid sequence encodes the isolated dual binding antibody comprising a VH domain comprising a set of CDRs, HCDR1, HCDR2, and HCDR3, wherein the amino acid sequence of HCDR1 is set forth in SEQ ID NO: 136, wherein the amino acid sequence of HCDR2 is set forth in SEQ ID NO: 137, wherein HX1 is W, wherein the amino acid sequence of HCDR3 is set forth in SEQ ID NO: 138, wherein HX2 is selected from the group consisting of A and S, HX3 is P, HX4 is Q, HX5 is W, HX6 is selected from the group consisting of E and M, HX7 is selected from the group consisting of L and W, HX8 is selected from the group consisting of V and T, HX9 is selected from the group consisting of H and A, HX10 is E, HX11 is A, HX12 is selected from the group I and L, and comprising a VL domain comprising a set of CDRs, LCDR1, LCDR2, and LCDR3, wherein the amino acid sequence of LCDR1 is set forth in SEQ ID NO: 139 wherein LX1 is L, LX2 is I, LX3 is L, wherein the amino acid sequence of LCDR2 is set forth as D D LX4, wherein LX4 is selected from the group consisting of S and G, wherein the amino acid sequence of LCDR3 is set forth in SEQ ID NO: 141 wherein LX5 is S, LX6 is S, and LX7 is selected from the group consisting of H and G.

In some embodiments, a nucleic acid construct comprising a nucleic acid sequence encodes a re-epitoped dual binding antibody comprising a VH domain comprising a set of CDRs, HCDR1, HCDR2, and HCDR3, and a VL domain comprising a set of CDRs, LCDR1, LCDR2, and LCDR3, wherein the amino acid sequences of each CDR are as set forth in FIGS. 1A and 1B for the clones set forth there, for example but not limited to: Clone C2: HCDR1 (SEQ ID NO: 136), HCDR2 (SEQ ID NO: 137) wherein HX1 is W, HCDR3 (SEQ ID NO: 138) wherein HX2 is A, HX3 is P, HX4 is Q, HX5 is W, HX6 is E, HX7 is L, HX8 is T, HX9 is A, HX10 is E, HX11 is A, HX12 is I, LCDR1 (SEQ ID NO: 139) wherein LX1 is L, LX2 is I, LX3 is L, LCDR2 (D D LX4) wherein LX4 is S), and LCDR3 (SEQ ID NO: 141) wherein LX5 is S, LX6 is S, and LX7 is G;

Clone C6: HCDR1 (SEQ ID NO: 136), HCDR2 (SEQ ID NO: 137) wherein HX1 is W, HCDR3 (SEQ ID NO: 138) wherein HX2 is A, HX3 is P, HX4 is Q, HX5 is W, HX6 is M, HX7 is L, HX8 is V, HX9 is A, HX10 is E, HX11 is A, HX12 is L, LCDR1 (SEQ ID NO: 139) wherein LX1 is L, LX2 is I, LX3 is L, LCDR2 (D D LX4, wherein LX4 is S, and LCDR3 (SEQ ID NO: 141) wherein LX5 is S, LX6 is S, and LX7 is H; or

Clone C9: HCDR1 (SEQ ID NO: 136), HCDR2 (SEQ ID NO: 137) wherein HX1 is W, HCDR3 (SEQ ID NO: 138) wherein HX2 is S, HX3 is P, HX4 is Q, HX5 is W, HX6 is E, HX7 is W, HX8 is V, HX9 is H, HX10 is E, HX11 is A, HX12 is L, LCDR1 (SEQ ID NO: 139) wherein LX1 is L, LX2 is I, LX3 is L, LCDR2 (D D LX4, wherein LX4 is G, and LCDR3 (SEQ ID NO: 141) wherein LX5 is S, LX6 is S, and LX7 is G.

In certain embodiments, a nucleic acid construct comprises a single nucleic acid sequence. In certain embodiments, a nucleic acid construct comprises two nucleic acid sequence. In certain embodiments, a nucleic acid construct comprises a single nucleic acid sequence, wherein a VH domain and a VL domain are encoded by the nucleic acid sequence. In certain embodiments, a nucleic acid construct comprises two nucleic acid sequences, wherein a VH domain is encoded by one nucleic acid sequence, and a VL domain is encoded by the other nucleic acid sequence.

A described herein, the present disclosure provides the polynucleotide sequences encoding the variant VH, VL, or both VH and VL domains described herein. In certain embodiments, the template VH domain is encoded by the nucleotide sequence set forth in SEQ ID NO: 55 and the template VL domain is encoded by the nucleotide sequence set forth in SEQ ID NO: 56.

In some embodiments, disclosed herein is nucleic acid construct, comprising a nucleic acid sequence encoding a dual binding antibody, said antibody comprising: a heavy chain variable region comprising the amino acid sequence set forth in SEQ ID NO: 1 with at least one amino acid variant at any of positions 52, 99, 100, 101, 102, 103, 104, 105, 106, 107, 108, or 111, or any combination thereof (IMGT positions: 57, 107, 108, 109, 110, 111, 111A, 112A, 112, 113, 114, or 117, or a combination thereof); a light chain variable region comprising the amino acid sequence set forth in SEQ ID NO: 2 with at least one amino acid variant at any of positions 26, 27, 31, 51, 56, 77, 92, 93, or 96, or any combination thereof (IMGT positions: 27, 28, 38, 65, 70, 94, 109, 110, or 115, or a combination thereof); or a combination of heavy chain variable region set forth in (a) and the light chain variable region set forth in (b); wherein the total number of variant positions in the encoded heavy chain variable region, the encoded light chain variable region, or a combination thereof, is at least 2.

In some embodiments, the nucleotide construct sequence comprises two nucleic acid sequences, one encoding a variant heavy chain variable region, and one a variant light chain variable region. In some embodiments, the nucleotide sequence or sequences encoding the dual binding antibody heavy chain variable region, light chain variable region, or both, is optimized for mammalian transcription and translation.

The present disclosure further provides, in certain embodiments, an isolated nucleic acid construct encoding the nucleic acid sequence as described herein. Illustrative polynucleotide sequence encoding variant VH and VL domains are provided in Table 2 below. Illustrative nucleic acid constructs, comprising a nucleic acid sequence encoding variant VH domains linked to VL domains are provided in Table 3 below.

Nucleic acids include DNA and RNA. These and related embodiments may include polynucleotides encoding the dual binding antibody as described herein. The term “isolated polynucleotide” as used herein shall mean a polynucleotide of genomic, cDNA, or synthetic origin, or some combination thereof, which by virtue of its origin the isolated polynucleotide (1) is not associated with all or a portion of a polynucleotide in which the isolated polynucleotide is found in nature, (2) is linked to a polynucleotide to which it is not linked in nature, or (3) does not occur in nature as part of a larger sequence.

A skilled artisan would appreciate that the terms “polynucleotide” and “nucleic acid sequence” may in some embodiments be used interchangeably having all the same meanings and qualities.

In some embodiments, isolated nucleic acid sequences disclosed herein, encode a VH domain comprising set of HCDRs as disclosed throughout and in FIG. 1A, a VL domain comprising set of a set of LCDRs as disclosed throughout and in FIG. 1B, a VH domain comprising a set of HCDRs and a VL domain comprising set of set of LCDRs as disclosed throughout and in FIGS. 1A and 1B, a VL domain or a VL domain, or a VH and a VL domain, of a dual binding antibody as described herein throughout in detail.

The term “polynucleotide” as used herein encompasses single-stranded or double-stranded nucleic acid polymers. In certain embodiments, the nucleotides comprising the polynucleotide can be ribonucleotides or deoxyribonucleotides or a modified form of either type of nucleotide. Said modifications include base modifications such as bromouridine, ribose modifications such as arabinoside and 2′,3′-dideoxyribose and internucleotide linkage modifications such as phosphorothioate, phosphorodithioate, phosphoroselenoate, phosphorodiselenoate, phosphoroanilothioate, phoshoraniladate and phosphoroamidate. The term “polynucleotide” specifically includes single and double stranded forms of DNA.

The term “naturally occurring nucleotide” includes deoxyribonucleotides and ribonucleotides. The term “modified nucleotide” includes nucleotides with modified or substituted sugar groups and the like. The term “oligonucleotide linkage” includes oligonucleotide linkages such as phosphorothioate, phosphorodithioate, phosphoroselenoate, phosphorodiselenoate, phosphoroanilothioate, phoshoraniladate, phosphoroamidate, and the like. See, e.g., LaPlanche et al., 1986, Nucl. Acids Res., 14:9081; Stec et al., 1984, J. Am. Chem. Soc., 106:6077; Stein et al., 1988, Nucl. Acids Res., 16:3209; Zon et al., 1991, Anti-Cancer Drug Design, 6:539; Zon et al., 1991, OLIGONUCLEOTIDES AND ANALOGUES: A PRACTICAL APPROACH, pp. 87-108 (F. Eckstein, Ed.), Oxford University Press, Oxford England; Stec et al., U.S. Pat. No. 5,151,510; Uhlmann and Peyman, 1990, Chemical Reviews, 90:543, the disclosures of which are hereby incorporated by reference for any purpose. An oligonucleotide can include a detectable label to enable detection of the oligonucleotide or hybridization thereof.

In other related embodiments, polynucleotide variants may have substantial identity to a polynucleotide template sequence, though the template sequence does not encode a dual binding antibody, or fragment thereof, or domain thereof.

In some embodiments, polynucleotide variants will contain one or more substitutions, additions, deletions and/or insertions, such that the binding affinity of a binding domain encoded by the variant polynucleotide newly binds to an epitope, relative to the unmodified template as specifically set forth herein.

In some embodiments, a nucleic acid sequence encodes the heavy chain variable region comprising the amino acid sequence set forth in SEQ ID NO: 1 with at least one amino acid variant at any of positions 52, 99, 100, 101, 102, 103, 104, 105, 106, 107, 108, or 111, or any combination thereof (IMGT positions: 57, 107, 108, 109, 110, 111, 111A, 112A, 112, 113, 114, or 117, or a combination thereof). In some embodiments, a nucleic acid sequence encodes the heavy chain variable region comprising the amino acid sequence set forth in SEQ ID NO: 1 with at least two amino acid variants at any of positions 52, 99, 100, 101, 102, 103, 104, 105, 106, 107, 108, or 111, or any combination thereof (IMGT positions: 57, 107, 108, 109, 110, 111, 111A, 112A, 112, 113, 114, or 117, or a combination thereof). In some embodiments, a nucleic acid sequence encodes the heavy chain variable region comprising the amino acid sequence set forth in SEQ ID NO: 1 with at least 1, 2, 3, 4, 5, 6, 7, 8, 9, or 10 amino acid variants at any of positions 52, 99, 100, 101, 102, 103, 104, 105, 106, 107, 108, or 111, or any combination thereof (IMGT positions: 57, 107, 108, 109, 110, 111, 111A, 112A, 112, 113, 114, or 117, ora combination thereof).

In some embodiments, the nucleic acid construct comprises a nucleic acid sequence encoding a heavy chain variable region comprising a sequence selected from the sequences set forth in SEQ ID Nos: 57, 59, 61, 63, 65, 67, 69, 71, 73, 75, 77, 79, 81, 83, 85, 87, 89, 91, 93, 95, 97, 99, 101, 105, and 107. In some embodiments, the nucleic acid sequence encoding a heavy chain variable region comprises the sequences set forth in SEQ ID Nos: 57. In some embodiments, the nucleic acid sequence encoding a heavy chain variable region comprises the sequences set forth in SEQ ID Nos: 59. In some embodiments, the nucleic acid sequence encoding a heavy chain variable region comprises the sequences set forth in SEQ ID Nos: 61. In some embodiments, the nucleic acid sequence encoding a heavy chain variable region comprises the sequences set forth in SEQ ID Nos: 63. In some embodiments, the nucleic acid sequence encoding a heavy chain variable region comprises the sequences set forth in SEQ ID Nos: 65. In some embodiments, the nucleic acid sequence encoding a heavy chain variable region comprises the sequences set forth in SEQ ID Nos: 67. In some embodiments, the nucleic acid sequence encoding a heavy chain variable region comprises the sequences set forth in SEQ ID Nos: 69. In some embodiments, the nucleic acid sequence encoding a heavy chain variable region comprises the sequences set forth in SEQ ID Nos: 71. In some embodiments, the nucleic acid sequence encoding a heavy chain variable region comprises the sequences set forth in SEQ ID Nos: 73. In some embodiments, the nucleic acid sequence encoding a heavy chain variable region comprises the sequences set forth in SEQ ID Nos: 75. In some embodiments, the nucleic acid sequence encoding a heavy chain variable region comprises the sequences set forth in SEQ ID Nos: 77. In some embodiments, the nucleic acid sequence encoding a heavy chain variable region comprises the sequences set forth in SEQ ID Nos: 79. In some embodiments, the nucleic acid sequence encoding a heavy chain variable region comprises the sequences set forth in SEQ ID Nos: 81. In some embodiments, the nucleic acid sequence encoding a heavy chain variable region comprises the sequences set forth in SEQ ID Nos: 83. In some embodiments, the nucleic acid sequence encoding a heavy chain variable region comprises the sequences set forth in SEQ ID Nos: 85. In some embodiments, the nucleic acid sequence encoding a heavy chain variable region comprises the sequences set forth in SEQ ID Nos: 87. In some embodiments, the nucleic acid sequence encoding a heavy chain variable region comprises the sequences set forth in SEQ ID Nos: 89. In some embodiments, the nucleic acid sequence encoding a heavy chain variable region comprises the sequences set forth in SEQ ID Nos: 91. In some embodiments, the nucleic acid sequence encoding a heavy chain variable region comprises the sequences set forth in SEQ ID Nos: 93. In some embodiments, the nucleic acid sequence encoding a heavy chain variable region comprises the sequences set forth in SEQ ID Nos: 95. In some embodiments, the nucleic acid sequence encoding a heavy chain variable region comprises the sequences set forth in SEQ ID Nos: 97. In some embodiments, the nucleic acid sequence encoding a heavy chain variable region comprises the sequences set forth in SEQ ID Nos: 99. In some embodiments, the nucleic acid sequence encoding a heavy chain variable region comprises the sequences set forth in SEQ ID Nos: 101. In some embodiments, the nucleic acid sequence encoding a heavy chain variable region comprises the sequences set forth in SEQ ID Nos: 105. In some embodiments, the nucleic acid sequence encoding a heavy chain variable region comprises the sequences set forth in SEQ ID Nos: 107.

In some embodiments, the nucleic acid construct comprises a nucleic acid sequence encoding a dual binding antibody heavy chain variable region sequences set forth in any of SEQ ID NOs: 4, 6, 8, 10, 12, 14, 16, 18, 20, 22, 24, 26, 28, 30, 32, 34, 36, 38, 40, 42, 44, 46, 48, 50, 52, and 54. In some embodiments, the nucleic acid sequence encodes a dual binding antibody heavy chain variable region sequence set forth in SEQ ID NO: 4. In some embodiments, the nucleic acid sequence encodes a dual binding antibody heavy chain variable region sequence set forth in SEQ ID NO: 6. In some embodiments, the nucleic acid sequence encodes a dual binding antibody heavy chain variable region sequence set forth in SEQ ID NO: 8. In some embodiments, the nucleic acid sequence encodes a dual binding antibody heavy chain variable region sequence set forth in SEQ ID NO: 10. In some embodiments, the nucleic acid sequence encodes a dual binding antibody heavy chain variable region sequence set forth in SEQ ID NO: 12. In some embodiments, the nucleic acid sequence encodes a dual binding antibody heavy chain variable region sequence set forth in SEQ ID NO: 14. In some embodiments, the nucleic acid sequence encodes a dual binding antibody heavy chain variable region sequence set forth in SEQ ID NO: 16. In some embodiments, the nucleic acid sequence encodes a dual binding antibody heavy chain variable region sequence set forth in SEQ ID NO: 18. In some embodiments, the nucleic acid sequence encodes a dual binding antibody heavy chain variable region sequence set forth in SEQ ID NO: 20. In some embodiments, the nucleic acid sequence encodes a dual binding antibody heavy chain variable region sequence set forth in SEQ ID NO: 22. In some embodiments, the nucleic acid sequence encodes a dual binding antibody heavy chain variable region sequence set forth in SEQ ID NO: 24. In some embodiments, the nucleic acid sequence encodes a dual binding antibody heavy chain variable region sequence set forth in SEQ ID NO: 26. In some embodiments, the nucleic acid sequence encodes a dual binding antibody heavy chain variable region sequence set forth in SEQ ID NO: 28. In some embodiments, the nucleic acid sequence encodes a dual binding antibody heavy chain variable region sequence set forth in SEQ ID NO: 30. In some embodiments, the nucleic acid sequence encodes a dual binding antibody heavy chain variable region sequence set forth in SEQ ID NO: 32. In some embodiments, the nucleic acid sequence encodes a dual binding antibody heavy chain variable region sequence set forth in SEQ ID NO: 34. In some embodiments, the nucleic acid sequence encodes a dual binding antibody heavy chain variable region sequence set forth in SEQ ID NO: 36. In some embodiments, the nucleic acid sequence encodes a dual binding antibody heavy chain variable region sequence set forth in SEQ ID NO: 38. In some embodiments, the nucleic acid sequence encodes a dual binding antibody heavy chain variable region sequence set forth in SEQ ID NO: 40. In some embodiments, the nucleic acid sequence encodes a dual binding antibody heavy chain variable region sequence set forth in SEQ ID NO: 42. In some embodiments, the nucleic acid sequence encodes a dual binding antibody heavy chain variable region sequence set forth in SEQ ID NO: 44. In some embodiments, the nucleic acid sequence encodes a dual binding antibody heavy chain variable region sequence set forth in SEQ ID NO: 46. In some embodiments, the nucleic acid sequence encodes a dual binding antibody heavy chain variable region sequence set forth in SEQ ID NO: 48. In some embodiments, the nucleic acid sequence encodes a dual binding antibody heavy chain variable region sequence set forth in SEQ ID NO: 50. In some embodiments, the nucleic acid sequence encodes a dual binding antibody heavy chain variable region sequence set forth in SEQ ID NO: 52. In some embodiments, the nucleic acid sequence encodes a dual binding antibody heavy chain variable region sequence set forth in SEQ ID NO: 54.

In some embodiments, the nucleic acid construct comprises a nucleic acid sequence encoding a dual binding antibody heavy chain variable region sequence set forth in Table 10 or Table 1; for example, the VH may comprise any one of SEQ ID NOs: 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345 and 347. In another embodiment, the nucleic acid construct comprises a nucleic acid sequence encoding a VH that is at least 80%, 85%, 90%, 95%, 98%, or 99% identical to the VH sequences disclosed herein.

In some embodiments, a nucleic acid sequence encodes the light chain variable region comprising the amino acid sequence set forth in SEQ ID NO: 2 with at least one amino acid variant at any of positions 26, 27, 31, 51, 56, 77, 92, 93, or 96, or any combination thereof (IMGT positions: 27, 28, 38, 65, 70, 94, 109, 110, or 115, or a combination thereof). In some embodiments, a nucleic acid sequence encodes the light chain variable region comprising the amino acid sequence set forth in SEQ ID NO: 2 with at least one amino acid variant at any of positions 26, 27, 31, 51, 56, 77, 92, 93, or 96, or any combination thereof (IMGT positions: 27, 28, 38, 65, 70, 94, 109, 110, or 115, or a combination thereof). In some embodiments, a nucleic acid sequence encodes the light chain variable region comprising the amino acid sequence set forth in SEQ ID NO: 2 with at least one amino acid variant at any of positions 26, 27, 31, 51, 56, 77, 92, 93, or 96, or any combination thereof (IMGT positions: 27, 28, 38, 65, 70, 94, 109, 110, or 115, or a combination thereof).

In some embodiments, the nucleic acid construct comprises a nucleic acid sequence encoding a light chain variable region comprising a sequence selected from the sequences set forth in SEQ ID NOs: 58, 60, 62, 64, 66, 68, 70, 72, 74, 76, 78, 80, 82, 84, 86, 88, 90, 92, 94, 96, 98, 100, 102, 104, 106, and 108. In some embodiments, the nucleic acid sequence encoding a light chain variable region comprises the sequences set forth in SEQ ID NO: 58. In some embodiments, the nucleic acid sequence encoding a light chain variable region comprises the sequences set forth in SEQ ID NO: 60. In some embodiments, the nucleic acid sequence encoding a light chain variable region comprises the sequences set forth in SEQ ID NO: 62. In some embodiments, the nucleic acid sequence encoding a light chain variable region comprises the sequences set forth in SEQ ID NO: 64. In some embodiments, the nucleic acid sequence encoding a light chain variable region comprises the sequences set forth in SEQ ID NO: 66. In some embodiments, the nucleic acid sequence encoding a light chain variable region comprises the sequences set forth in SEQ ID NO: 68. In some embodiments, the nucleic acid sequence encoding a light chain variable region comprises the sequences set forth in SEQ ID NO: 70. In some embodiments, the nucleic acid sequence encoding a light chain variable region comprises the sequences set forth in SEQ ID NO: 72. In some embodiments, the nucleic acid sequence encoding a light chain variable region comprises the sequences set forth in SEQ ID NO: 74. In some embodiments, the nucleic acid sequence encoding a light chain variable region comprises the sequences set forth in SEQ ID NO: 76. In some embodiments, the nucleic acid sequence encoding a light chain variable region comprises the sequences set forth in SEQ ID NO: 78. In some embodiments, the nucleic acid sequence encoding a light chain variable region comprises the sequences set forth in SEQ ID NO: 80. In some embodiments, the nucleic acid sequence encoding a light chain variable region comprises the sequences set forth in SEQ ID NO: 82. In some embodiments, the nucleic acid sequence encoding a light chain variable region comprises the sequences set forth in SEQ ID NO: 84. In some embodiments, the nucleic acid sequence encoding a light chain variable region comprises the sequences set forth in SEQ ID NO: 86. In some embodiments, the nucleic acid sequence encoding a light chain variable region comprises the sequences set forth in SEQ ID NO: 88. In some embodiments, the nucleic acid sequence encoding a light chain variable region comprises the sequences set forth in SEQ ID NO: 90. In some embodiments, the nucleic acid sequence encoding a light chain variable region comprises the sequences set forth in SEQ ID NO: 92. In some embodiments, the nucleic acid sequence encoding a light chain variable region comprises the sequences set forth in SEQ ID NO: 94. In some embodiments, the nucleic acid sequence encoding a light chain variable region comprises the sequences set forth in SEQ ID NO: 96. In some embodiments, the nucleic acid sequence encoding a light chain variable region comprises the sequences set forth in SEQ ID NO: 98. In some embodiments, the nucleic acid sequence encoding a light chain variable region comprises the sequences set forth in SEQ ID NO: 100. In some embodiments, the nucleic acid sequence encoding a light chain variable region comprises the sequences set forth in SEQ ID NO: 102. In some embodiments, the nucleic acid sequence encoding a light chain variable region comprises the sequences set forth in SEQ ID NO: 104. In some embodiments, the nucleic acid sequence encoding a light chain variable region comprises the sequences set forth in SEQ ID NO: 106. In some embodiments, the nucleic acid sequence encoding a light chain variable region comprises the sequences set forth in SEQ ID NO: 108.

In some embodiments, the nucleic acid construct comprises a nucleic acid sequence encoding a dual binding antibody light chain variable region sequences set forth in any of SEQ ID NOs: 3, 5, 7, 9, 11, 13, 15, 17, 19, 21, 23, 25, 27, 29, 31, 33, 35, 37, 39, 41, 43, 45, 47, 49, 51, and 53. In some embodiments, the nucleic acid sequence encodes a dual binding antibody light chain variable region sequence set forth in SEQ ID NO: 3. In some embodiments, the nucleic acid sequence encodes a dual binding antibody light chain variable region sequence set forth in SEQ ID NO: 5. In some embodiments, the nucleic acid sequence encodes a dual binding antibody light chain variable region sequence set forth in SEQ ID NO: 7. In some embodiments, the nucleic acid sequence encodes a dual binding antibody light chain variable region sequence set forth in SEQ ID NO: 9. In some embodiments, the nucleic acid sequence encodes a dual binding antibody light chain variable region sequence set forth in SEQ ID NO: 11. In some embodiments, the nucleic acid sequence encodes a dual binding antibody light chain variable region sequence set forth in SEQ ID NO: 13. In some embodiments, the nucleic acid sequence encodes a dual binding antibody light chain variable region sequence set forth in SEQ ID NO: 15. In some embodiments, the nucleic acid sequence encodes a dual binding antibody light chain variable region sequence set forth in SEQ ID NO: 17. In some embodiments, the nucleic acid sequence encodes a dual binding antibody light chain variable region sequence set forth in SEQ ID NO: 19. In some embodiments, the nucleic acid sequence encodes a dual binding antibody light chain variable region sequence set forth in SEQ ID NO: 21. In some embodiments, the nucleic acid sequence encodes a dual binding antibody light chain variable region sequence set forth in SEQ ID NO: 23. In some embodiments, the nucleic acid sequence encodes a dual binding antibody light chain variable region sequence set forth in SEQ ID NO: 25. In some embodiments, the nucleic acid sequence encodes a dual binding antibody light chain variable region sequence set forth in SEQ ID NO: 27. In some embodiments, the nucleic acid sequence encodes a dual binding antibody light chain variable region sequence set forth in SEQ ID NO: 29. In some embodiments, the nucleic acid sequence encodes a dual binding antibody light chain variable region sequence set forth in SEQ ID NO: 31. In some embodiments, the nucleic acid sequence encodes a dual binding antibody light chain variable region sequence set forth in SEQ ID NO: 33. In some embodiments, the nucleic acid sequence encodes a dual binding antibody light chain variable region sequence set forth in SEQ ID NO: 35. In some embodiments, the nucleic acid sequence encodes a dual binding antibody light chain variable region sequence set forth in SEQ ID NO: 37. In some embodiments, the nucleic acid sequence encodes a dual binding antibody light chain variable region sequence set forth in SEQ ID NO: 39. In some embodiments, the nucleic acid sequence encodes a dual binding antibody light chain variable region sequence set forth in SEQ ID NO: 41. In some embodiments, the nucleic acid sequence encodes a dual binding antibody light chain variable region sequence set forth in SEQ ID NO: 43. In some embodiments, the nucleic acid sequence encodes a dual binding antibody light chain variable region sequence set forth in SEQ ID NO: 45. In some embodiments, the nucleic acid sequence encodes a dual binding antibody light chain variable region sequence set forth in SEQ ID NO: 47. In some embodiments, the nucleic acid sequence encodes a dual binding antibody light chain variable region sequence set forth in SEQ ID NO: 49. In some embodiments, the nucleic acid sequence encodes a dual binding antibody light chain variable region sequence set forth in SEQ ID NO: 51. In some embodiments, the nucleic acid sequence encodes a dual binding antibody light chain variable region sequence set forth in SEQ ID NO: 53.

In some embodiments, the nucleic acid construct comprises a nucleic acid sequence encoding a dual binding antibody light chain variable region sequence set forth in Table 10 or Table 1; for example, the VL may comprise one of SEQ ID NOs: 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346 and 348. In another embodiment, the nucleic acid construct comprises a nucleic acid sequence encoding a VL that is at least 80%, 85%, 90%, 95%, 98%, or 99% identical to the VL sequences disclosed herein.

In some embodiments, a nucleic acid construct comprises a nucleic acid sequence encoding a dual binding antibody heavy chain variable region-light chain variable region pair, said nucleic acid sequence selected from the paired sequences set forth in SEQ ID NOs: 57 and 58, SEQ ID Nos: 59 an 60, SEQ ID Nos: 61 and 62, SEQ ID Nos: 63 and 64, SEQ ID Nos: 65 and 66, SEQ ID Nos: 67 and 68, SEQ ID Nos: 69 and 70, SEQ ID Nos: 71 and 72, SEQ ID Nos: 73 and 74, SEQ ID Nos: 75 and 76, SEQ ID Nos: 77 and 78, SEQ ID Nos: 79 and 80, SEQ ID Nos: 81 and 82, SEQ ID Nos: 83 and 84, SEQ ID Nos: 85 and 86, SEQ ID Nos: 87 and 88, SEQ ID Nos: 89 and 90, SEQ ID Nos: 91 and 92, SEQ ID Nos: 93 and 94, SEQ ID Nos: 95 and 96, SEQ ID Nos: 97 and 98, SEQ ID Nos: 99 and 100, SEQ ID Nos: 101 and 102, SEQ ID Nos: 103 and 104, SEQ ID Nos: 105 and 106, and SEQ ID Nos: 107 and 108.

In some embodiments, a nucleic acid construct comprises a nucleic acid sequence encoding a dual binding antibody heavy chain variable region-light chain variable region pair as shown in Table 10 or Table 1; for example, the VH and VL pair can be one of the following: SEQ ID NOs:209 and 210, SEQ ID NOs:211 and 212, SEQ ID NOs:213 and 214, SEQ ID NOs:215 and 216, SEQ ID NOs:217 and 218, SEQ ID NOs:219 and 220, SEQ ID NOs:221 and 222, SEQ ID NOs:223 and 224, SEQ ID NOs:225 and 226, SEQ ID NOs:227 and 228, SEQ ID NOs:229 and 230, SEQ ID NOs:231 and 232, SEQ ID NOs:233 and 234, SEQ ID NOs:235 and 236, SEQ ID NOs:237 and 238, SEQ ID NOs:239 and 240, SEQ ID NOs:241 and 242, SEQ ID NOs:243 and 244, SEQ ID NOs:245 and 246, SEQ ID NOs:247 and 248, SEQ ID NOs:249 and 250, SEQ ID NOs:251 and 252, SEQ ID NOs:253 and 254, SEQ ID NOs:255 and 256, SEQ ID NOs:257 and 258, SEQ ID NOs:259 and 260, SEQ ID NOs:261 and 262, SEQ ID NOs:263 and 264, SEQ ID NOs:265 and 266, SEQ ID NOs:267 and 268, SEQ ID NOs:269 and 270, SEQ ID NOs:271 and 272, SEQ ID NOs:273 and 274, SEQ ID NOs:275 and 276, SEQ ID NOs:277 and 278, SEQ ID NOs:279 and 280, SEQ ID NOs:281 and 282, SEQ ID NOs:283 and 284, SEQ ID NOs:285 and 286, SEQ ID NOs:287 and 288, SEQ ID NOs:289 and 290, SEQ ID NOs:291 and 292, SEQ ID NOs:293 and 294, SEQ ID NOs:295 and 296, SEQ ID NOs:297 and 298, SEQ ID NOs:299 and 300, SEQ ID NOs:301 and 302, SEQ ID NOs:303 and 304, SEQ ID NOs:305 and 306, SEQ ID NOs:307 and 308, SEQ ID NOs:309 and 310, SEQ ID NOs:311 and 312, SEQ ID NOs:313 and 314, SEQ ID NOs:315 and 316, SEQ ID NOs:317 and 318, SEQ ID NOs:319 and 320, SEQ ID NOs:321 and 322, SEQ ID NOs:323 and 324, SEQ ID NOs:325 and 326, SEQ ID NOs:327 and 328, SEQ ID NOs:329 and 330, SEQ ID NOs:331 and 332, SEQ ID NOs:333 and 334, SEQ ID NOs:335 and 336, SEQ ID NOs:337 and 338, SEQ ID NOs:339 and 340, SEQ ID NOs:341 and 342, SEQ ID NOs:343 and 344, SEQ ID NOs:345 and 346, SEQ ID NOs:347 and 348. In another embodiment, the VH and VL pair is at least 80%, 85%, 90%, 95%, 98%, or 99% identical to the VH and VL sequences disclosed herein.

A skilled artisan would appreciate that in some embodiments, the sequence encoding a VH domain and the sequence encoding the VL domain are linked by a sequence encoding a linker sequence. In some embodiments, a nucleic acid sequence encodes a polypeptide linker. ggcggtggtggtagcggaggcggaggatcaggtggaggcggcagt (SEQ ID NO: 148).

In some embodiments, a nucleic acid construct comprises a nucleic acid sequence encoding a dual binding antibody heavy chain variable region-light chain variable region scFv, said nucleic acid sequence selected from the sequences set forth in SEQ ID NOs: 109-135.

In some embodiments, a nucleic acid construct comprising a nucleic acid sequence encoding a dual antibody described herein, encodes an IgG immunoglobulin. In some embodiments, a nucleic acid sequence encoding a dual antibody encodes an IgG1 immunoglobulin, an IgG2 immunoglobulin, an IgG3 immunoglobulin, or an IgG4 immunoglobulin. In some embodiments, a nucleic acid sequence encoding a dual antibody encodes an IgG1 immunoglobulin. In some embodiments, a nucleic acid sequence encoding a dual antibody encodes an IgG2 immunoglobulin. In some embodiments, a nucleic acid sequence encoding a dual antibody encodes an IgG3 immunoglobulin. In some embodiments, a nucleic acid sequence encoding a dual antibody encodes an IgG4 immunoglobulin. In some embodiments, a nucleic acid sequence encoding a dual antibody encodes an IgG1 immunoglobulin or an IgG4 immunoglobulin.

In some embodiments, a nucleic acid sequence encoding a dual antibody encodes an Fab immunoglobulin fragment. In some embodiments, a nucleic acid sequence encoding a dual antibody encodes an F(ab′)2 immunoglobulin fragment. In some embodiments, a nucleic acid sequence encoding a dual antibody encodes an Fv immunoglobulin construct. In some embodiments, a nucleic acid sequence encoding a dual antibody encodes an scFv immunoglobulin construct. In some embodiments, a nucleic acid sequence encoding a dual antibody encodes a minibody immunoglobulin construct comprising a pair of single-chain Fv fragments, which are linked via CH3 domains.

In some embodiments, a nucleic acid sequence encoding a dual antibody encodes a diabody immunoglobulin construct. In some embodiments, a diabody immunoglobulin construct comprises a heavy chain variable (VH) and light chain variable (VL) regions connected by a small peptide linker. In some embodiments, a diabody immunoglobulin construct comprises single-chain (Fv) 2 in which two scFv fragments are covalently linked to each other. In some embodiments, a nucleic acid sequence encoding a dual antibody encodes a diabody immunoglobulin construct comprising three scFv fragments covalently linked to each other.

In some embodiments, an isolated polynucleotide construct encodes an isolated dual binding antibody, as disclosed herein.

In some embodiments, a nucleic acid sequence encoding a dual antibody encodes a mutated immunoglobulin. In some embodiments, a nucleic acid sequence encoding a dual antibody encodes a mutant IgG unable to bind antibody-dependent cellular cytotoxicity components. In some embodiments, a nucleic acid sequence encoding a dual antibody encodes a mutant IgG1 unable to bind antibody-dependent cellular cytotoxicity components. In some embodiments, a nucleic acid sequence encoding a dual binding antibody encodes an IgG comprising the L₂₃₄A/L₂₃₅A (LALA) mutations. In some embodiments, a nucleic acid sequence encoding a dual binding antibody encodes an IgG1 comprising the L₂₃₄A/L₂₃₅A/P329G (LALAPG) mutations.

In some embodiments, as disclosed herein, a mutagenesis approach, such as site-specific mutagenesis, may be employed for the preparation of variants VH, VL, or VH and VL nucleic acid sequences encoding the variant VH, VL, or VH and VL amino acid sequences. Template VH and VL nucleic acid sequences SEQ ID NO: 55 and 56, respectively, encode the template amino acid sequences SEQ ID NO: 1 and SEQ ID NO: 2, respectively. In some embodiments, a dual binding antibody comprises a variant VH domain, a variant VL domain, or both, encoded by a variant VH, VL, or VH and VL nucleotide sequences, wherein said nucleotide sequence comprises site-specific mutagenesis of the nucleotide template sequences SEQ ID NO: 55 and SEQ ID NO: 56, respectively. By this approach, specific modifications in a polypeptide sequence can be made through mutagenesis of the underlying polynucleotides that encode them. These techniques provide a straightforward approach to prepare and test sequence variants, for example but not limited to, introducing one or more nucleotide sequence changes into the polynucleotide in view of the amino acid variant sites desired, as described above in detail.

Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Mutations may be employed in a selected polynucleotide sequence to improve, alter, decrease, modify, or otherwise change the properties of the polynucleotide itself, and/or alter the properties, activity, composition, stability, or primary sequence of the encoded polypeptide.

In certain embodiments, mutagenesis of the polynucleotide sequences that encode component parts of the dual binding antibody (VH domains, VL domain, or a combination thereof, as disclosed herein, is contemplated in order to alter the binding properties of the encoded template VH or VL or both, such that the resulting antibody comprises a dual binding affinity. The techniques of site-specific mutagenesis are well-known in the art and are widely used to create variants of both polypeptides and polynucleotides. For example, site-specific mutagenesis is often used to alter a specific portion of a DNA molecule. In such embodiments, a primer comprising typically about 14 to about 25 nucleotides or so in length is employed, with about 5 to about 10 residues on both sides of the junction of the sequence being altered.

As will be appreciated by those of skill in the art, site-specific mutagenesis techniques have often employed a phage vector that exists in both a single stranded and double stranded form. Typical vectors useful in site-directed mutagenesis include vectors such as the M13 phage. These phages are readily commercially available and their use is generally well-known to those skilled in the art. Double-stranded plasmids are also routinely employed in site directed mutagenesis that eliminates the step of transferring the gene of interest from a plasmid to a phage.

In general, site-directed mutagenesis in accordance herewith is performed by first obtaining a single-stranded vector or melting apart of two strands of a double-stranded vector that includes within its sequence a DNA sequence that encodes the desired peptide. An oligonucleotide primer bearing the desired mutated sequence is prepared, generally synthetically. This primer is then annealed with the single-stranded vector and subjected to DNA polymerizing enzymes such as E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells, such as E. coli cells, and clones are selected which include recombinant vectors bearing the mutated sequence arrangement.

The preparation of sequence variants of the selected peptide-encoding DNA segments using site-directed mutagenesis provides a means of producing potentially useful species and is not meant to be limiting as there are other ways in which sequence variants of peptides and the DNA sequences encoding them may be obtained. In some embodiments, methods of preparing libraries includes those known in the art, for example but not limited to methods described in U.S. Pat. No. 9,889,423, which are included herein in their entirety. In some embodiments, a method for designing the sequence variants within a library comprises designing the variant sequences on a computer and then having the sequence synthesized, a method that involves both chemical and biochemical processes.

As used herein, the term “oligonucleotide directed mutagenesis procedure” encompasses template-dependent processes and vector-mediated propagation which result in an increase in the concentration of a specific nucleic acid molecule relative to its initial concentration, or in an increase in the concentration of a detectable signal, such as amplification. As used herein, the term “oligonucleotide directed mutagenesis procedure” encompasses a process that involves the template-dependent extension of a primer molecule. The term “template dependent process” encompasses nucleic acid synthesis of an RNA or a DNA molecule wherein the sequence of the newly synthesized strand of nucleic acid is dictated by the well-known rules of complementary base pairing (see, for example, Watson, 1987). Typically, vector mediated methodologies involve the introduction of the nucleic acid fragment into a DNA or RNA vector, the clonal amplification of the vector, and the recovery of the amplified nucleic acid fragment. Examples of such methodologies are provided by U.S. Pat. No. 4,237,224, specifically incorporated herein by reference in its entirety.

In another approach for the production of polypeptide VH and VL variants, recursive sequence recombination, as described in U.S. Pat. No. 5,837,458, may be employed. In this approach, iterative cycles of recombination and screening or selection are performed to “evolve” individual polynucleotide variants having, for example, increased binding affinity. Certain embodiments also provide constructs in the form of plasmids, vectors, transcription or expression cassettes which comprise at least one polynucleotide as described herein.

In certain embodiments, the polynucleotides described above, e.g., VH, VL, or VH and VL variant polynucleotides, fragments and hybridizing sequences, encoding the amino acid VH, VL, or VH and VL variants, are comprised in a dual biding antibody.

The polynucleotides described herein, or fragments thereof, regardless of the length of the coding sequence itself, may be combined with other DNA sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant DNA protocol. For example, illustrative polynucleotide segments with total lengths of about 10,000, about 5000, about 3000, about 2,000, about 1,000, about 500, about 200, about 100, about 50 base pairs in length, and the like, (including all intermediate lengths) are contemplated to be useful.

In certain embodiments, the isolated polynucleotide is inserted into a vector. In some embodiments, a vector comprises an expression vector comprising a polynucleotide construct disclosed herein.

The term “vector” as used herein encompasses a vehicle into which a polynucleotide encoding a protein may be covalently inserted so as to bring about the expression of that protein and/or the cloning of the polynucleotide. The isolated polynucleotide may be inserted into a vector using any suitable methods known in the art, for example, without limitation, the vector may be digested using appropriate restriction enzymes and then may be ligated with the isolated polynucleotide having matching restriction ends.

Examples of suitable vectors include, without limitation, plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses. Examples of categories of animal viruses useful as vectors include, without limitation, retrovirus (including lentivirus), adenovirus, adeno-associated virus, herpesvirus (e.g., herpes simplex virus), poxvirus, baculovirus, papillomavirus, and papovavirus (e.g., SV40).

For expression of the dual antibody or components thereof, the vector may be introduced into a host cell to allow expression of the polypeptide within the host cell. The expression vectors may contain a variety of elements for controlling expression, including without limitation, promoter sequences, transcription initiation sequences, enhancer sequences, selectable markers, and signal sequences. These elements may be selected as appropriate by a person of ordinary skill in the art. In some embodiments, these elements may be considered “control” elements.

A skilled artisan would appreciate that the term “control sequence” may encompass polynucleotide sequences that can affect expression, processing or intracellular localization of coding sequences to which they are ligated or operably linked. The nature of such control sequences may depend upon the host organism. In particular embodiments, transcription control sequences for prokaryotes may include a promoter, ribosomal binding site, and transcription termination sequence. In other particular embodiments, transcription control sequences for eukaryotes may include promoters comprising one or a plurality of recognition sites for transcription factors, transcription enhancer sequences, transcription termination sequences and polyadenylation sequences. In certain embodiments, “control sequences” can include leader sequences and/or fusion partner sequences.

In some embodiments, for example but not limited to, the promoter sequences may be selected to promote the transcription of the polynucleotide in the vector. Suitable promoter sequences include, without limitation, T7 promoter, T3 promoter, SP6 promoter, beta-actin promoter, EF 1a promoter, CMV promoter, and SV40 promoter Enhancer sequences may be selected to enhance the transcription of the polynucleotide. Selectable markers may be selected to allow selection of the host cells inserted with the vector from those not, for example, the selectable markers may be genes that confer antibiotic resistance. Signal sequences may be selected to allow the expressed polypeptide to be transported outside of the host cell.

A vector may also include materials to aid in its entry into the cell, including but not limited to a viral particle, a liposome, or a protein coating. In some embodiments, a host cell comprises an expression vector disclosed herein.

In some embodiments, an expression vector comprises an isolated nucleic acid sequence encoding a dual antibody or a component thereof, for example but not limited to a VH domain, a VL domain, a combined VH-VL domain as may be present in Fab elements, F(ab′)2 elements, an scFv, an Fv, a minibody, a diabody, or a triabody, as described above. Dual binding domains and the components thereof have been described in detail above.

In some embodiments, an expression vector comprises an isolated nucleic acid sequence encoding a VH domain. In some embodiments, an expression vector comprises an isolated nucleic acid sequence encoding a VL domain. In some embodiments, an expression vector comprises an isolated nucleic acid sequence encoding a VH and a VL domain. In some embodiments, an expression vector comprises an isolated nucleic acid sequence encoding two VH and VL domains. In some embodiments, an expression vector comprises an isolated nucleic acid sequence encoding three VH and VL domains.

In some embodiments, an expression vector comprises an isolated nucleic acid sequence encoding a VH domain component of a dual antibody. In some embodiments, an expression vector comprises an isolated nucleic acid sequence encoding a VL domain component of a dual antibody. In some embodiments, an expression vector comprises an isolated nucleic acid sequence encoding VH and VL domain components of a dual antibody.

In some embodiments, an expression vector comprises an isolated nucleic acid sequence encoding a VH domain component of a dual IgG antibody or a fragment thereof. In some embodiments, an expression vector comprises an isolated nucleic acid sequence encoding a VL domain component of a dual IgG antibody or a fragment thereof. In some embodiments, an expression vector comprises an isolated nucleic acid sequence encoding VH and VL domain components of a dual IgG antibody or a fragment thereof.

In some embodiments, an expression vector comprises an isolated nucleic acid sequence encoding a VH domain component of a scFv. In some embodiments, an expression vector comprises an isolated nucleic acid sequence encoding a VL domain component of a scFv. In some embodiments, an expression vector comprises an isolated nucleic acid sequence encoding VH and VL domain components of a scFv.

Dual binding antibodies have been described in detail above. The skilled artisan, using the knowledge in the art and specific details newly described herein would surely appreciate the range of components that may be encoded by an isolated nucleic acid described herein.

For cloning of the polynucleotide, the vector may be introduced into a host cell (an isolated host cell) to allow replication of the vector itself and thereby amplify the copies of the polynucleotide contained therein. The cloning vectors may contain sequence components generally include, without limitation, an origin of replication, promoter sequences, transcription initiation sequences, enhancer sequences, and selectable markers. These elements may be selected as appropriate by a person of ordinary skill in the art. For example, the origin of replication may be selected to promote autonomous replication of the vector in the host cell.

In certain embodiments, the present disclosure provides isolated host cells containing the vector provided herein. The host cells containing the vector may be useful in expression or cloning of the polynucleotide(s) contained in the vector.

In some embodiments, a recombinant host cell comprises one or more constructs as described above. A nucleic acid encoding any CDR or set of CDR's or VH domain or VL domain or antibody antigen-binding site or antibody molecule, for example but not limited to an IgG, an Fv, an scFv, an Fab, an F(ab′)₂, a minibody, a diabody, or a triabody. In some embodiments, disclosed herein is a method of production of the encoded product, which method comprises expression from encoding nucleic acid constructs. Expression may in some embodiments, be achieved by culturing under appropriate conditions recombinant host cells containing the nucleic acid construct. Following production by expression a VH or VL domain, or a VH-VL pair, or an antibody, may be isolated and/or purified using any suitable technique, then used as appropriate, for example in methods of treatment as described herein.

In some embodiments, dual binding antibodies, VH and/or VL domains, and encoding nucleic acid molecules and vectors according to the present invention may be prepared and isolated and/or purified, in substantially pure or homogeneous form.

In some embodiments, systems for cloning and expression of a polypeptide in a variety of different host cells are well known. Suitable host cells can include, without limitation, prokaryotic cells, fungal cells, yeast cells, or higher eukaryotic cells such as insect cells or mammalian cells.

Suitable prokaryotic cells for this purpose include, without limitation, eubacteria, such as Gram-negative or Gram-positive organisms, for example, Enterobactehaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis, Pseudomonas such as P. aeruginosa, and Streptomyces.

The expression of antibodies and antigen-binding fragments in prokaryotic cells such as E. coli is well established in the art. For a review, see for example Pluckthun, A. Bio/Technology 9: 545-551 (1991). Expression in eukaryotic cells in culture is also available to those skilled in the art as an option for production of antibodies or antigen-binding fragments thereof, see recent reviews, for example Ref, M. E. (1993) Curr. Opinion Biotech. 4: 573-576; Trill J. J. et al. (1995) Curr. Opinion Biotech 6: 553-560.

Suitable fungal cells for this purpose include, without limitation, filamentous fungi and yeast. Illustrative examples of fungal cells include, Saccharomyces cerevisiae, common baker's yeast, Schizosaccharomyces pombe, Kluyveromyces hosts such as, eg., K. lactis, K fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Higher eukaryotic cells, in particular, those derived from multicellular organisms can be used for expression of glycosylated VH and VL domains, as provided herein. Suitable higher eukaryotic cells include, without limitation, invertebrate cells and insect cells, and vertebrate cells. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruiffly), and Bombyx mori have been identified. A variety of viral strains for transfection are publicly available, e.g., the K-1 variant of Autographa californica NPV and the Bm-5 strain of Bombyx mori NPV, and such viruses may be used as the virus herein as described herein, particularly for transfection of Spodoptera frugiperda cells. Plant cell cultures of cotton, corn, potato, soybean, petunia, tomato, and tobacco can also be utilized as hosts Mammalian cell lines available in the art for expression of a heterologous polypeptide include Chinese hamster ovary (CHO) cells, HeLa cells, baby hamster kidney cells, NS0 mouse melanoma cells, YB2/0 rat myeloma cells, human embryonic kidney cells, human embryonic retina cells and many others. Non-limiting examples of vertebrate cells include mammalian host cell lines such as monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); ExpiCHO-S(TM) cells (ThermoFisher Scientific cat. #A29133); mouse Sertoli cells (TM4, Mather, Biol. Reprod. 23:243-251 (1980)); monkey kidney cells (CV1 ATCC CCL 70); African green monkey kidney cells (VERO-76, ATCC CRK-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2).

In some embodiments, an expression vector comprises a nucleic acid construct described herein. Suitable vectors can be chosen or constructed, containing appropriate regulatory sequences, including promoter sequences, terminator sequences, polyadenylation sequences, enhancer sequences, marker genes and other sequences as appropriate. Regulatory sequences may be operably linked to the nucleic acid sequence(s) comprised within a nucleic acid construct. Vectors may be plasmids, viral e.g. ‘phage, or phagemid, as appropriate. For further details see, for example, Molecular Cloning: a Laboratory Manu^{al}: 3rd edition, Sambrook and Russell, 2001, Cold Spring Harbor Laboratory Press. Many known techniques and protocols for manipulation of nucleic acid, for example in preparation of nucleic acid constructs, mutagenesis, sequencing, introduction of DNA into cells and gene expression, and analysis of proteins, are described in detail in Current Protocols in Molecular Biology, Second Edition, Ausubel et al. eds., John Wiley & Sons, 1988, Short Protocols in Molecular Biology: A Compendium of Methods from Current Protocols in Molecular Biology, Ausubel et al. eds., John Wiley & Sons, 4.sup.th edition 1999. The disclosures of Sambrook et al. and Ausubel et al. (both) are incorporated herein by reference.

The vector can be introduced to the host cell using any suitable methods known in the art, including, without limitation, DEAE-dextran mediated delivery, calcium phosphate precipitate method, cationic lipids mediated delivery, liposome mediated transfection, electroporation, microprojectile bombardment, receptor-mediated gene delivery, delivery mediated by polylysine, histone, chitosan, and peptides. Standard methods for transfection and transformation of cells for expression of a vector of interest are well known in the art.

In some embodiments, provided herein is a host cell containing nucleic acid as disclosed herein. Such a host cell may be in vitro and may be in culture. Such a host cell may be in vivo. In vivo presence of the host cell may allow intracellular expression of the dual binding antibodies described herein, as “intrabodies” or intracellular antibodies. Intrabodies may be used for gene therapy.

In certain embodiments, the host cells comprise a first vector encoding a first polypeptide, e.g., a VH domain, and a second vector encoding a second polypeptide, e.g., a VL domain. In certain embodiments, the host cells comprise a vector encoding a first polypeptide, e.g., a VH domain, and a second polypeptide, e.g., a VL domain.

In certain embodiments, the host cells comprise a first vector encoding a variant VH domain and a second vector encoding a variant VL domain. In certain embodiments, the host cells comprise a single vector encoding a variant VH domain and a variant VL domain.

In some embodiments, an isolated cell comprises an isolated nucleic acid sequence, as disclosed herein. In some embodiments, an isolated cell comprises two isolated nucleic acid sequences as disclosed herein, wherein one nucleic acid encodes a variant VH domain and the other nucleic acid encodes a variant VL domain. In some embodiments, an isolated cell comprises a single isolated nucleic acid sequence as disclosed herein, that encodes a variant VH domain and a variant VL domain.

In certain embodiments, a first vector and a second vector may or may not be introduced simultaneously. In certain embodiments, the first vector and the second vector may be introduced together into the host cell. In certain embodiments, the first vector may be introduced first into the host cell, and then the second vector may be introduced. In certain embodiments, the first vector may be introduced into the host cell, which is then established into a stable cell line expressing the first polypeptide, and then the second vector may be introduced into the stable cell line.

In certain embodiments, the host cells comprise a vector encoding for at least one variant VH domain and at least one a variant VL comprised within a dual binding antibody.

The introduction may be followed by causing or allowing expression from the nucleic acid, e.g. by culturing host cells under conditions for expression of the gene. In certain embodiments, the present disclosure provides methods of expressing the polypeptide provided herein, comprising culturing the host cell containing the vector under conditions in which the inserted polynucleotide in the vector is expressed.

In some embodiments, the nucleic acid is integrated into the genome (e.g. chromosome) of the host cell. Integration may be promoted by inclusion of sequences which promote recombination with the genome, in accordance with standard techniques. In some embodiments, the nucleic acid construct is not integrated into the genome and the vector is episomal.

In some embodiments, disclosed herein is a method which comprises using a construct as stated above in an expression system in order to express a dual binding antibody or fragment thereof, as described herein above.

Suitable conditions for expression of the polynucleotide may include, without limitation, suitable medium, suitable density of host cells in the culture medium, presence of necessary nutrients, presence of supplemental factors, suitable temperatures and humidity, and absence of microorganism contaminants A person with ordinary skill in the art can select the suitable conditions as appropriate for the purpose of the expression.

### Methods of Synthesizing an Engineered, “Re-Epitoped” Dual Antibody

In some embodiments, described herein is a method of producing a dual binding antibody comprising a VH domain comprising HCDRs as described herein. In some embodiments, described herein is a method of producing a dual binding antibody comprising a VL domain comprising LCDRs as described herein. In some embodiments, described herein is a method of producing a dual binding antibody comprising a VH domain comprising HCDRs as described herein and a VL domain comprising LCDRs as described herein.

In some embodiments, a method of producing a dual binding antibody a heavy chain variable region comprising: (a) the amino acid sequence set forth in SEQ ID NO: 1 with at least one amino acid variant at any of positions 52, 99, 100, 101, 102, 103, 104, 105, 106, 107, 108, or 111, or any combination thereof (IMGT positions: 57, 107, 108, 109, 110, 111, 111A, 112A, 112, 113, 114, or 117, or a combination thereof); (b) a light chain variable region comprising the amino acid sequence set forth in SEQ ID NO: 2 with at least one amino acid variant at any of positions 26, 27, 31, 51, 56, 77, 92, 93, or 96, or any combination thereof (IMGT positions: 27, 28, 38, 65, 70, 94, 109, 110, or 115, or a combination thereof); or (c) a combination of the heavy chain variable region set forth in (a) and the light chain variable region set forth in (b); wherein the total number of variant positions in said heavy chain variable region, said light chain variable region, or said combination thereof of said dual binding antibody, is at least 2; comprises steps of culturing a cell or cells comprising a nucleic acid sequence encoding at least a VH and a VL of the dual binding antibody, wherein polypeptides comprising the variant VH and variant VL domains are expressed and isolated, and wherein said isolated variant VH and variant VL domains form a heterodimer. As disclosed herein in detail, the isolated nucleic acid sequences encoding variant VH and variant VL domains may be comprised within vectors, wherein the same vector or different vectors are used. In some embodiments, each variant VH domain and variant VL domain may be expressed from a different host cell, wherein dimerization occurs following isolation or purification of the component variant VH and variant VL domains. In some embodiments variant VH and variant VL domains may be expressed from a same host cell, wherein dimerization occurs in culture or following isolation or purification of the component variant VH and variant VL domains.

A skilled artisan would appreciate that producing a dual binding antibody comprises synthesizing amino acid polypeptide components comprising VH domains, VL domains, or both. In some embodiments, said synthesis starts from a nucleic acid construct as described herein in detail. The terms “producing” and “synthesizing” may in some embodiments, be used herein interchangeably having all the same qualities and meanings.

In some embodiments, synthesizing a dual binding antibody comprises synthesizing an IgG heavy chain comprising a variant VH domain, synthesizing an IgG light chain comprising a variant VL domain, or both. In some embodiments, synthesizing a dual binding antibody comprises synthesizing an IgG heavy chain comprising a variant VH domain. In some embodiments, synthesizing a dual binding antibody comprises synthesizing an IgG light chain comprising a variant VL domain. In some embodiments, synthesizing a dual binding antibody comprises synthesizing both an IgG heavy chain comprising a variant VH domain and an IgG light chain comprising a variant VL domain. In some embodiments, synthesizing a dual binding antibody comprises synthesizing an Fab comprising a fragment of an IgG heavy chain comprising a variant VH domain and a fragment of an IgG light chain comprising a variant VL domain. In some embodiments, synthesizing a dual binding antibody comprises synthesizing an F(ab′)2 comprising a fragment of an IgG heavy chain comprising a variant VH domain and a fragment of an IgG light chain comprising a variant VL domain. In some embodiments, synthesizing a dual binding antibody comprises synthesizing an Fv comprising a variant VH domain and a variant VL domain. In some embodiments, synthesizing a dual binding antibody comprises synthesizing a scFv comprising a variant VH domain and a variant VL domain. In some embodiments, synthesizing a dual binding antibody comprises synthesizing a minibody comprising a variant VH domain and a variant VL domain. In some embodiments, synthesizing a dual binding antibody comprises synthesizing a diabody comprising a variant VH domain and a variant VL domain. In some embodiments, synthesizing a dual binding antibody comprises synthesizing a triabody comprising a variant VH domain and a variant VL domain. In some embodiments, synthesizing a dual binding antibody comprises synthesizing a variant VH domain. In some embodiments, synthesizing an dual binding antibody comprises synthesizing a variant VL domain.

In certain embodiments, the polypeptide expressed in the host cell can form a dimer and thus produce a dual binding antibody or the binding component thereof.

In some embodiments, methods of synthesizing a dual binding antibody comprise a step of mutating a nucleic acid sequence encoding a template heavy chain variable region that does not comprise a dual binding VH domain in order to create a variant VH domain that may comprise a dual binding VH domain. In some embodiments, methods of synthesizing a dual binding antibody comprise a step of mutating a nucleic acid sequence encoding a template light chain variable region that does not comprise a dual binding VL domain in order to create a variant VL domain that may comprise a dual binding VL domain. In some embodiments, methods of synthesizing a dual binding antibody comprise a step of mutating a nucleic acid sequence encoding a template heavy chain variable region that does not comprise a dual binding VH domain in order to create a variant VH domain that may comprise a dual binding VH domain, and mutating a nucleic acid sequence encoding a template light chain variable region that does not comprise a dual binding VL domain in order to create a variant VL domain that may comprise a dual binding VL domain, wherein the variant VH and VL domains comprise a dual variable region of an antibody. Methods of mutating nucleic acid sequences have been described in detail above and are exemplified below in the Examples.

In some embodiments, a template nucleic acid sequence encoding the template heavy chain variable region is set forth in SEQ ID NO: 55. In some embodiments, a template nucleic acid sequence encoding the template light chain variable region is set forth in SEQ ID NO: 56. As stated throughout, template VH and VL sequences do not comprise dual binding regions.

In some embodiments, methods of synthesizing a dual binding antibody comprise introducing at least 2 variant sites within VH and VL domains. In some embodiments, methods of synthesizing a dual binding antibody comprise introducing at least 3, 4, 5, 6, 7, 8, 9, 10, 11, 12, 13, 14, or 15 variant sites within VH and VL domains Variant sites may be distributed between the VH domain and the VL domain. In some embodiments, variant sites are within CDR regions of a VH domain. In some embodiments, variant sites are within CDR regions of a VL domain. In some embodiments, variant sites are within FR regions of a VH domain. In some embodiments, variant sites are within FR regions of a VL domain. In some embodiments, variant sites are within CDR and or FR regions of a VH domain. In some embodiments, variant sites are within CDR and or FR regions of a VL domain. In some embodiments, variant sites are within CDR and or FR regions of a VH domain, and within CDR and or FR regions of a VL domain.

In certain embodiments, the variant VH and variant VL domains complex may be formed inside the host cell. For example, the variant VH and variant VL domains heterodimer may be formed inside the host cell with the aid of relevant enzymes and/or cofactors. In certain embodiments, the variant VH and variant VL domains polypeptide complex may be secreted out of the cell. In certain embodiments, the variant VH and variant VL domains may be secreted out of the host cell and form a heterodimer outside of the host cell.

In certain embodiments, the variant VH and variant VL domains may be separately expressed and allowed to dimerize under suitable conditions. For example, the variant VH and variant VL domains may be combined in a suitable buffer and allow the variant VH and variant VL domains to dimerize through appropriate interactions such as hydrophobic interactions. For another example, variant VH and variant VL domains may be combined in a suitable buffer containing an enzyme and/or a cofactor which can promote the dimerization of the variant VH and variant VL domains. For another example, the variant VH and variant VL domains may be combined in a suitable vehicle and allow them to react with each other in the presence of a suitable reagent and/or catalyst.

In certain embodiments, the variant VH and variant VL domains may be comprised within longer polypeptide sequences, which may include for example but not limited to constant regions, hinge regions, linker regions, Fc regions, or disulfide binding regions, or any combination thereof. A constant domain is an immunoglobulin fold unit of the constant part of an immunoglobulin molecule, also referred to as a domain of the constant region (e.g. CH1, CH2, CH3, CH4, Ck, Cl). In some embodiments, the longer polypeptide may comprise multiple copies of a variant VH domain, a variant VL domain, or both, for example but not limited to when the dual binding antibody comprises a diabody or a triabody.

In certain embodiments, the variant VH and variant VL domains are generated by DNA synthesis and PCR, and translation of nucleotide sequences generated thereof. In certain embodiments, the generated sequences may be subcloned into an expression vector. In certain embodiments, the generated sequences may be subcloned into two expression vectors. In certain embodiments, said expression vector is a plasmid. In certain embodiments, said the variant VH and variant VL domains are constructed on an IgG template, wherein said IgG template does not have dual binding capabilities.

In certain embodiments, transient expression is performed by co-transfecting the expression vector encoding the variant VH and variant VL domains or by transfecting an expression vector encoding both into a suitable cell. A skilled artisan would appreciate that there are a number of transfection methods and protocols that can be used for this purpose. In certain embodiments, transfection or co-transfection is executed using the PEI method.

The expressed polypeptides comprising the variant VH and variant VL domains and/or the polypeptide complex can be collected using any suitable methods. The variant VH and variant VL domains and/or the polypeptide complex can be expressed intracellularly, in the periplasmic space or be secreted outside of the cell into the medium. If the polypeptides comprising variant VH and variant VL domains and/or the polypeptide complex is expressed intracellularly, the host cells containing the polypeptides comprising variant VH and variant VL domains and/or the polypeptide complex may be lysed and polypeptide and/or the polypeptide complex may be isolated from the lysate by removing the unwanted debris by centrifugation or ultrafiltration. If the polypeptides comprising variant VH and variant VL domains and/or the polypeptide complex is secreted into periplasmic space of E. coli, the cell paste may be thawed in the presence of agents such as sodium acetate (pH 3.5), EDTA, and phenylmethylsulfonylfluoride (PMSF) over about 30 min, and cell debris can be removed by centrifugation (Carter et al., BioTechnology 10:163-167 (1992)). If the polypeptides comprising variant VH and variant VL domains and/or the polypeptide complex is secreted into the medium, the supernatant of the cell culture may be collected and concentrated using a commercially available protein concentration filter, for example, an Amincon or Millipore Pellicon ultrafiltration unit. A protease inhibitor and/or an antibiotic may be included in the collection and concentration steps to inhibit protein degradation and/or growth of contaminated microorganisms.

The expressed polypeptides comprising variant VH and variant VL domains and/or the polypeptide complex can be further purified by a suitable method, such as without limitation, affinity chromatography, hydroxylapatite chromatography, size exclusion chromatography, gel electrophoresis, dialysis, ion exchange fractionation on an ion-exchange column, ethanol precipitation, reverse phase HPLC, chromatography on silica, chromatography on heparin Sepharose, chromatography on an anion or cation exchange resin (such as a polyaspartic acid column), chromatofocusing, SDS-PAGE, and ammonium sulfate precipitation (see, for review, Bonner, P. L., Protein purification, published by Taylor & Francis, 2007; Janson, J. C., et al, Protein purification: principles, high resolution methods and applications, published by Wiley-VCH, 1998).

In certain embodiments, the polypeptides comprising variant VH and variant VL domains and/or polypeptide dimer complexes can be purified by affinity chromatography. In certain embodiments, protein A chromatography or protein A/G (fusion protein of protein A and protein G) chromatography can be useful for purification of polypeptides and/or polypeptide complexes comprising a component derived from antibody CH2 domain and/or CH3 domain (Lindmark et al., J. Immunol Meth. 62:1-13 (1983)); Zettlit, K. A., Antibody Engineering, Part V, 531-535, 2010). In certain embodiments, a dual binding antibody disclosed herein does not bind to protein A. In certain embodiments, protein G chromatography can be useful for purification of polypeptides and/or polypeptide complexes comprising IgGγ3 heavy chain (Guss et al., EMBO J. 5:1567 1575 (1986)). In certain embodiments, protein L chromatography can be useful for purification of polypeptides and/or polypeptide complexes comprising K light chain (Sudhir, P., Antigen engineering protocols, Chapter 26, published by Humana Press, 1995; Nilson, B. H. K. et al, J. Biol. Chem., 267, 2234-2239 (1992)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a CH3 domain, the Bakerbond ABX resin (J. T. Baker, Phillipsburg, N.J.) is useful for purification.

Following any preliminary purification step(s), the mixture comprising the dual binding antibody and contaminants may be subjected to low pH hydrophobic interaction chromatography using an elution buffer at a pH between about 2.5-4.5, preferably performed at low salt concentrations (e.g., from about 0-0.25M salt).

In certain embodiments, the polypeptides comprising variant VH and variant VL domains and/or polypeptide dimer complexes can be purified by affinity chromatography and size exclusion chromatography (SEC). A skilled artisan would appreciate that there are a number of methods and protocols suitable for this purpose. In certain embodiments, protein purification by affinity chromatography and SEC is performed using an AKTA pure instrument (GE Lifesciences). In certain embodiments, affinity capture of the dual binding antibody is achieved by passing the harvested supernatants over a column of CaptureSelect™ CH1-XL Affinity Matrix (Thermo Scientific). After washing column with PBS, the protein is eluted with 0.1M Glycine, pH 2.5, and immediately neutralized with ⅙ volume of 1M Tris-HCl, pH 8.0. The affinity purified protein is then concentrated to 5-10 mg/ml using Amicon 30 kD concentrator (Merck Millipore) and subjected to SEC purification on a Superdex®200 column (GE Lifesciences) equilibrated with PBS. Protein fractions are then collected and analyzed using SDS-PAGE and HPLC-SEC.

Binding to an epitope of the synthesized dual binding immunoglobulins may be analyzed using well known methods in the art, as described herein, including ELISA analysis, SPR analysis, DSF analysis, and cell-based binding assays.

In some embodiments, a method of synthesizing a dual binding antibody comprising: a heavy chain variable region comprising a template amino acid sequence set forth in SEQ ID NO: 1, wherein said template comprises at least one amino acid variant at any of positions 52, 99, 100, 101, 102, 103, 104, 105, 106, 107, 108, or 111, or any combination thereof (IMGT positions: 57, 107, 108, 109, 110, 111, 111A, 112A, 112, 113, 114, or 117, or a combination thereof); and a light chain variable region comprising a template amino acid sequence set forth in SEQ ID NO: 2, wherein said template comprises at least one amino acid variant at any of positions 26, 27, 31, 51, 56, 77, 92, 93, or 96, or any combination thereof (IMGT positions: 27, 28, 38, 65, 70, 94, 109, 110, or 115, or a combination thereof); wherein the total number of variant positions in said heavy chain variable region, said light chain variable region, or the combination thereof, is at least 2; comprises the following steps:
(a) mutating the template heavy chain variable region, the template light chain variable region, or both,
(i) wherein said mutating said template heavy variable chain region comprises mutating the template heavy chain variable region set forth in SEQ ID NO: 1, wherein said selected template variable chain does not comprise a dual binding region,
(ii) wherein said mutating said template light chain variable region comprises mutating the template light chain variable region set forth in SEQ ID NO: 2, wherein said selected template variable chain does not comprise a dual binding region;
(iii) wherein said mutating both said template heavy variable chain region and said template light chain variable region comprises mutating the template heavy chain variable region set forth in SEQ ID NO: 1 and mutating the template light chain variable region set forth in SEQ ID NO: 2, wherein said selected template variable chains together do not comprise a dual binding region, and wherein said mutating comprises mutating at least two residue position in said template heavy chain variable region, said template light chain variable region or a combination thereof;
(b) synthesizing the mutated template variant heavy chain variable chain and the mutated template variant light chain variable chain;
(c) formatting said mutated template variant heavy chain variable chain and said mutated template variant light chain variable chain into a human antibody format; and
(d) screening the human antibody of (c) for binding to dual antigens;
thereby producing a dual binding antibody.

As described herein and exemplified below, in some embodiments, that antibody synthesized comprises an IgG immunoglobulin. In some embodiments, the antibody synthesized comprises an IgG1 immunoglobulin, an IgG2 immunoglobulin, an IgG3 immunoglobulin, or an IgG4 immunoglobulin. In some embodiments, the antibody synthesized comprises an IgG1 immunoglobulin. In some embodiments, the antibody synthesized comprises an IgG2 immunoglobulin. In some embodiments, the antibody synthesized comprises an IgG3 immunoglobulin. In some embodiments, the antibody synthesized comprises an IgG4 immunoglobulin. In some embodiments, the antibody synthesized comprises an IgG1 immunoglobulin or an IgG4 immunoglobulin.

In some embodiments, the antibody synthesized comprises an Fab immunoglobulin fragment. In some embodiments, the antibody synthesized comprises an F(ab′)₂ immunoglobulin fragment. In some embodiments, the antibody synthesized comprises an Fv immunoglobulin construct In some embodiments, the antibody synthesized comprises an scFv immunoglobulin construct In some embodiments, the antibody synthesized comprises a minibody immunoglobulin construct comprising a pair of single-chain Fv fragments which are linked via CH3 domains.

In some embodiments, the antibody synthesized comprises a diabody immunoglobulin construct. In some embodiments, the antibody synthesized comprises a diabody immunoglobulin construct comprising three scFv fragments covalently linked to each other. In some embodiments, the antibody synthesized comprises a triabody.

In some embodiments, the antibody synthesized comprises a mutated IgG that is unable to bind antibody-dependent cellular cytotoxicity components. In some embodiments, the antibody synthesized comprises a mutated IgG1 that is unable to bind antibody-dependent cellular cytotoxicity components. In some embodiments, the antibody synthesized comprises a mutated IgG4 that is unable to bind antibody-dependent cellular cytotoxicity components.

### Immunoglobulin Libraries

In certain embodiments, disclosed herein is a library of immunoglobulins or fragments thereof, comprising variant VH domains, variant VL domains, or variant VH and VL domains, as described herein in detail (See, Examples below). A library of immunoglobulins or fragments thereof comprising a variant VH domains, a variant VL domains, or variant VH and VL domains, may in some embodiments, be screened for dual binding antibodies, fragments thereof, or components thereof.

In some embodiments, a library of immunoglobulins or fragments thereof, comprises a library of variable heavy chain domains. In some embodiments, a library of immunoglobulins or fragments thereof, comprises a library of variable light chain domains. In some embodiments, a library of immunoglobulins or fragments thereof, comprises a library of variable heavy chain domains and variable light chain domains.

In some embodiments, a method for generating a library of dual antigen binding immunoglobulin variable heavy chain regions, for screening for binding to an epitope comprises: (a) selecting the VH template antigen-binding molecule set forth in SEQ ID NO: 1, wherein said selected template does not specifically bind an epitope; (b) selecting at least one residue position from positions 52, 99, 100, 101, 102, 103, 104, 105, 106, 107, 108, or 111, or any combination thereof (IMGT positions: 57, 107, 108, 109, 110, 111, 111A, 112A, 112, 113, 114, or 117, or a combination thereof) in said template SEQ ID NO: 1, for mutation; and (c) selecting at least one variant residue to substitute at the at least one residue position selected in (b); such that a library containing a plurality of variants of said template VH is generated. In some embodiments, a method for generating a library of dual antigen binding immunoglobulin variable light chain regions, for screening for binding to an epitope comprises: (a) selecting the VL template antigen-binding molecule set forth in SEQ ID NO: 2, wherein said selected template does not specifically bind an epitope; (b) selecting at least one residue position from positions 26, 27, 31, 51, 56, 77, 92, 93, or 96, or any combination thereof (IMGT positions: 27, 28, 38, 65, 70, 94, 109, 110, or 115, or a combination thereof) in said template SEQ ID NO: 2, for mutation; and (c) selecting at least one variant residue to substitute at the at least one residue position selected in (b); such that a library containing a plurality of variants of said template VL is generated.

In some embodiments, a method for generating a library of dual antigen binding immunoglobulin comprising variable heavy chain regions and variable light chain regions, for screening for binding to an epitope comprises: (a) selecting the VH template antigen-binding molecule set forth in SEQ ID NO: 1, wherein said selected template does not specifically bind an epitope; (b) selecting the VL template antigen-binding molecule set forth in SEQ ID NO: 2, wherein said selected template does not specifically bind an epitope; (c) selecting at least one residue position from positions 52, 99, 100, 101, 102, 103, 104, 105, 106, 107, 108, or 111, or any combination thereof (IMGT positions: 57, 107, 108, 109, 110, 111, 111A, 112A, 112, 113, 114, or 117, or a combination thereof) in said template SEQ ID NO: 1, for mutation; (d) selecting at least one residue position from positions 26, 27, 31, 51, 56, 77, 92, 93, or 96, or any combination thereof (IMGT positions: 27, 28, 38, 65, 70, 94, 109, 110, or 115, or a combination thereof) in said template SEQ ID NO: 2, for mutation; and (e) selecting at least one variant residue to substitute at the at least one residue position selected in (c) or selecting at least one variant residue to substitute at the at least one residue position selected in (d), such that the total number of variant residues in each potentially dual binding immunoglobulins is a least 2, and such that a library containing a plurality of variants of said template VH and variants of said template VL is generated.

In some embodiments, methods for constructing a library can be found in the examples. In some embodiments, a library generated as described herein, may be used to identify immunoglobulins binding to dual targets. In some embodiments, a library generated as described herein, may be used to identify immunoglobulins binding to specific epitopes.

In some embodiments, use of a protein library comprising an immunoglobulin comprising a variant VH, a variant VL, or a variant VH and variant VL as described herein in detail, provides a method to identify immunoglobulins binding to dual targets. In some embodiments, use of a protein library comprising an immunoglobulin comprising a variant VH, a variant VL, or a variant VH and variant VL as described herein in detail, provides a method to identify immunoglobulins binding to specific epitopes.

In some embodiments, a protein library comprising an immunoglobulin comprising a variant VH and variant VL comprises a library of antibody molecules. In some embodiments, a protein library comprising an immunoglobulin comprising a variant VH and variant VL comprises a library of IgG molecules. In some embodiments, a protein library comprising an immunoglobulin comprising a variant VH and variant VL comprises a library of IgG1, IgG2, IgG3, or IgG4 molecules. In some embodiments, the IgG molecule is a mutant IgG molecule, unable to bind antibody-dependent cellular cytotoxicity components.

In some embodiments, a protein library comprising an immunoglobulin comprising a variant VH and variant VL comprises a library of Fab or F(ab′)₂ molecules. In some embodiments, a protein library comprising an immunoglobulin comprising a variant VH and variant VL comprises a library of Fv molecules, scFv molecules, minibody molecules, diabody molecules, or triabody molecules.

In some embodiments, existing immunoglobulin VH and VL templates can be changed to introduce variant amino acids at specific positions with the goal of generating dual antigen binding sites in said variant VH and VL domains, wherein a protein library of the variant VH and VL domains comprises at least 10; 100; 1,000; 10,000; 100,000; or 1,000,000 variant VH, variant VL, or variant VH and variant VL domains with at least two variant positions. In some embodiments, a protein library of the variant VH and VL domains comprises between 1,000 to 1,000,000 variant VH, variant VL, or variant VH and variant VL domains with at least two variant positions. In some embodiments, a protein library of the variant VH and VL domains comprises between 10,000 to 1,000,000 variant VH, variant VL, or variant VH and variant VL domains with at least two variant positions.

In some embodiments, a protein library of the variant VH and VL domains comprises between 1,000 to 1,000,000 variant VH, variant VL, or variant VH and variant VL domains with at least 2, 3, 4, 5, 6, 7, 8, 9, 10, 11, 12, 13, 14, 15, or more variant positions. In some embodiments, a protein library of the variant VH and VL domains comprises between 10,000 to 1,000,000 variant VH, variant VL, or variant VH and variant VL domains with at least 2, 3, 4, 5, 6, 7, 8, 9, 10, 11, 12, 13, 14, 15, or more variant positions.

In some embodiments, a protein library of the variant VH and VL domains comprises between 10⁶ to 10¹⁴ variant VH, variant VL, or variant VH and variant VL domains with at least 2, 3, 4, 5, 6, 7, 8, 9, 10, 11, 12, 13, 14, 15, or more variant positions. In some embodiments, a protein library of the variant VH and VL domains comprises between 10⁶ to 10¹⁴ variant VH, variant VL, or variant VH and variant VL domains with at least 2, 3, 4, 5, 6, 7, 8, 9, 10, 11, 12, 13, 13, 14, 15, or more variant positions.

The library is then screened for binding to one or more antigens. After molecular characterization for the desired properties a selected antibody domain or region, for example but not limited to a VH or VL domain or both, is cloned into an immunoglobulin molecule by genetic engineering techniques, so that it replaces the corresponding region. Alternatively, only the DNA coding for the VH, or VL, or both regions, or coding for the mutated amino acids may be exchanged to obtain an immunoglobulin with the additional binding site for a molecule. In some embodiments, selection of the immunoglobulin molecule into which the variant regions are cloned, may be selected from an IgG, an Fv, an scFv, an Fab, an F(ab′)₂, a minibody, a diabody, or a triabody. In some embodiments, an IgG is an IgG1, an IgG2, an IgG3, or an IgG4. In some embodiments, an IgG comprises a mutant IgG unable to bind antibody-dependent cellular cytotoxicity components.

In some embodiments, the CDRs expressed are as described above for HCDR1, HCDR2, HCD3, LCDR1, LCDR2, and LCDR3, wherein certain positions comprise variant amino acids, as described in detail above and is shown in FIGS. 1A and 1B.

The sites for mutation are describe above, and in certain embodiments include from positions 52, 99, 100, 101, 102, 103, 104, 105, 106, 107, 108, or 111, or any combination thereof (IMGT positions: 57, 107, 108, 109, 110, 111, 111A, 112A, 112, 113, 114, or 117, or a combination thereof) in said template SEQ ID NO: 1, and positions 26, 27, 31, 51, 56, 77, 92, 93, or 96, or any combination thereof (IMGT positions: 27, 28, 38, 65, 70, 94, 109, 110, or 115, or a combination thereof) in said template SEQ ID NO: 2. In some embodiments, additional sites within said VH template or said VL template may be mutated.

In certain embodiments, the method of generating a library further comprises synthesizing the template variants (VH, VL, or both VH and VL) from said nucleic acid constructs, described above in detail, to form the library.

The result of generating a library as described above comprises a library of immunoglobulins comprising: (a) a heavy chain variable region comprising the amino acid sequence set forth in SEQ ID NO: 1 with at least one amino acid variant at any of positions 52, 99, 100, 101, 102, 103, 104, 105, 106, 107, 108, or 111, or any combination thereof (IMGT positions: 57, 107, 108, 109, 110, 111, 111A, 112A, 112, 113, 114, or 117, or a combination thereof); (b) a light chain variable region comprising the amino acid sequence set forth in SEQ ID NO: 2 with at least one amino acid variant at any of positions 26, 27, 31, 51, 56, 77, 92, 93, or 96, or any combination thereof (IMGT positions: 27, 28, 38, 65, 70, 94, 109, 110, or 115, or a combination thereof); or (c) a combination of the heavy chain variable region set forth in (a) and the light chain variable region set forth in (b); wherein the total number of variant positions in the heavy chain variable region, the light chain variable region, or a combination thereof is at least 2.

Mammalian cell expression systems have been described above. These expression systems offer a number of potential advantages for therapeutic antibody generation to create a library of potential dual binding immunoglobulins, including the ability to co-select for key manufacturing-related properties such as high-level expression and stability, while displaying functional glycosylated IgGs on the cell surface.

In some embodiments, a library of immunoglobulins comprises IgG molecules, Fab molecules, F(ab′)₂ molecules, FV molecules, VH molecules, VL molecules, scFv molecules, diabodies, minibodies, or triabodies. In some embodiments, an IgG molecule comprises an IgG1, an IgG2, an IgG3, or an IgG4. In some embodiments, an IgG comprises a mutated IgG that is unable to bind antibody-dependent cellular cytotoxicity components. In some embodiments, an IgG1 comprises a mutated IgG1 that is unable to bind antibody-dependent cellular cytotoxicity components.

In some embodiments, disclosed herein are methods directed to screening the library with antigen molecules or a portion thereof, in order to select for dual-binding molecules that have desired properties (e.g., binding affinity, stability, etc.). In some embodiments, a portion of an antigen comprises an at least one IL-13 antigenic epitope. In some embodiments, disclosed herein is a dual-binding molecule isolated from the library after said screening.

In some embodiments, disclosed herein is a method for screening a library of immunoglobulins as described, for dual-binding molecules, comprising: (a) screening the library with an antigen molecules or fragment thereof to identify dual-binding molecules that bind said epitopes of interest; (b) sequencing the binders identified in step (a) to determine which residues are variants and which variant residues are enriched in the binding immunoglobulins; (c) using the information from step (b) to synthesize an optimized library of variants of the dual binders; and (d) repeating steps (a)-(c) using the optimized library. In some embodiments, disclosed herein is a method for screening a library of immunoglobulins as described, for dual-binding molecules, comprising: (a) screening the library with epitopes of interest to identify dual-binding molecules that bind said epitope of interest; (b) sequencing the binders identified in step (a) to determine which residues are variants and which variant residues are enriched in the binding immunoglobulins; (c) using the information from step (b) to synthesize an optimized library of variants of the dual binders; and (d) repeating steps (a)-(c) using the optimized library.

According to some embodiments, the specific binding of the variant immunoglobulins to the antigen molecule is determined by a binding assay selected from the group consisting of immunological assays, including but not limited to enzyme linked immunosorbent assays (ELISA), surface plasmon resonance assays, saturation transfer difference nuclear magnetic resonance spectroscopy, transfer NOE (trNOE) nuclear magnetic resonance spectroscopy, competitive assays, tissue binding assays, live cell binding assays and cellular extract assays.

Binding assays can be carried out using a variety of methods known in the art, including but not limited to FRET (Fluorescence Resonance Energy Transfer) and BRET (Bioluminescence Resonance Energy Transfer)-based assays, AlphaScreen.™. (Amplified Luminescent Proximity Homogeneous Assay), Scintillation Proximity Assay, ELISA (Enzyme-Linked Immunosorbent Assay), SPR (Surface Plasmon Resonance, also known as BIACORE®), isothermal titration calorimetry, differential scanning calorimetry, gel electrophoresis, and chromatography including gel filtration. These and other methods may take advantage of some fusion partner or label.

The variant immunoglobulin is, in some embodiments, conjugated to a label selected from the group consisting of organic molecules, enzyme labels, radioactive labels, colored labels, fluorescent labels, chromogenic labels, luminescent labels, haptens, digoxigenin, biotin, metal complexes, metals, colloidal gold and mixtures thereof. Conjugation to a label may in certain embodiments, allow the simple detection of said conjugate in, for instance, binding assays (e.g. ELISA) and binding studies.

### Compositions of Use

In some embodiments, described herein are pharmaceutical compositions comprising the dual binding antibody, as described herein in detail, which provides a therapeutic agent. In some embodiments, described herein are pharmaceutical compositions comprising the dual binding antibody comprising a therapeutic agent comprising a mutant IgG unable to bind antibody-dependent cellular cytotoxicity components. In some embodiments, described herein are pharmaceutical compositions comprising a dual binding antibody having therapeutic properties against allergic or respiratory conditions.

In some embodiments, a pharmaceutical composition comprises a dual binding antibody comprising a variant VH, a variant VL, or a variant VH and a variant VL, and a pharmaceutically acceptable carrier. The amino acid sequences of variant VH and variant VL domains, and pair thereof, have been described in detail above (See for example, but not limited to Table 1).

In certain embodiments, a composition comprises any of the isolated dual binding antibodies disclosed herein, and a pharmaceutically acceptable carrier.

In one embodiment, the pharmaceutical composition comprises a dual binding antibody having HCDR1, HCDR2 and HCDR3 comprising the amino acid sequence of SEQ ID NOs:349, 350 and 351 respectively, and LCDR1, LCDR2 and LCDR3 comprising the amino acid sequence of SEQ ID NO: 359, D D V, and SEQ ID NO: 361, respectively.

In another embodiment, the pharmaceutical composition comprises a dual binding antibody having HCDR1, HCDR2 and HCDR3 comprising the amino acid sequence of SEQ ID NOs:349, 356 and 351 respectively, and LCDR1, LCDR2 and LCDR3 comprising the amino acid sequence of SEQ ID NO: 364, D D V, and SEQ ID NO: 371, respectively.

In another embodiment, the pharmaceutical composition comprises a dual binding antibody having HCDR1, HCDR2 and HCDR3 comprising the amino acid sequence of SEQ ID NOs:349, 350 and 351 respectively, and LCDR1, LCDR2 and LCDR3 comprising the amino acid sequence of SEQ ID NO: 362, D D V, and SEQ ID NO: 384 respectively.

In another embodiment, the pharmaceutical composition comprises a dual binding antibody having HCDR1, HCDR2 and HCDR3 comprising the amino acid sequence of SEQ ID NOs:349, 350 and 351 respectively, and LCDR1, LCDR2 and LCDR3 comprising the amino acid sequence SEQ ID NO: 364, D D V, and SEQ ID NO: 384, respectively.

In another embodiment, the pharmaceutical composition comprises a dual binding antibody having HCDR1, HCDR2 and HCDR3 comprising the amino acid sequence as shown in Table 8 or Table 4, and LCDR1, LCDR2 and LCDR3 comprising the amino acid sequence as shown in Table 9 or Table 5.

In another embodiment, the pharmaceutical composition comprises a dual binding antibody comprising a heavy chain variable domain (VH) and a light chain variable domain (VL), wherein the VH and VL comprise the amino acid sequences of SEQ ID Nos:209 and 210.

In another embodiment, the pharmaceutical composition comprises a dual binding antibody comprising a heavy chain variable domain (VH) and a light chain variable domain (VL), wherein the VH and VL comprise the amino acid sequences of SEQ ID Nos:219 and 220.

In another embodiment, the pharmaceutical composition comprises a dual binding antibody comprising a heavy chain variable domain (VH) and a light chain variable domain (VL), wherein the VH and VL comprise the amino acid sequences of SEQ ID Nos:249 and 250.

In another embodiment, the pharmaceutical composition comprises a dual binding antibody comprising a heavy chain variable domain (VH) and a light chain variable domain (VL), wherein the VH and VL comprise the amino acid sequences of SEQ ID Nos:337 and 338.

In another embodiment, the pharmaceutical composition comprises a dual binding antibody comprising a heavy chain variable domain (VH) and a light chain variable domain (VL), wherein the VH and VL comprise the amino acid sequences as shown in Table 10 or Table 1.

In another embodiment, the pharmaceutical composition comprises a dual binding antibody comprising a heavy chain variable domain (VH) and a light chain variable domain (VL), wherein the VH and VL are at least 80%, 85%, 90%, 95%, 98%, or 99% identical to the VH and VL sequences disclosed herein.

In some embodiments, a pharmaceutical composition comprising a dual binding antibody comprises any dual antibody described herein comprising a variant VH, a variant VL, or a variant VH and a variant VL. In some embodiments, a pharmaceutical composition comprising a dual binding antibody comprises a heavy chain variable region comprising the amino acid sequence set forth in SEQ ID NO: 1 with at least one amino acid variant at any of positions 52, 99, 100, 101, 102, 103, 104, 105, 106, 107, 108, or 111, or any combination thereof (IMGT positions: 57, 107, 108, 109, 110, 111, 111A, 112A, 112, 113, 114, or 117, or a combination thereof); a light chain variable region comprising the amino acid sequence set forth in SEQ ID NO: 2 with at least one amino acid variant at any of positions 26, 27, 31, 51, 56, 77, 92, 93, or 96, or any combination thereof (IMGT positions: 27, 28, 38, 65, 70, 94, 109, 110, or 115, or a combination thereof); or a combination of the heavy chain variable region set forth in (a) and the light chain variable region set forth in (b); wherein the total number of variant positions in the heavy chain variable region, the light chain variable region, or a combination thereof is at least 2.

A skilled artisan would recognize that in some embodiments, the term “dual binding antibody” may be used interchangeably with the term “drug” or “agent” having all the same meanings and qualities. In some embodiments, a drug comprising a dual binding antibody comprises a pharmaceutical composition.

In some embodiments, described herein are compositions comprising the dual binding antibody as described herein and administration of such composition in a variety of therapeutic settings.

Administration of the dual binding antibodies described herein, in pure form or in an appropriate pharmaceutical composition, can be carried out via any of the accepted modes of administration of agents for serving similar utilities. The pharmaceutical compositions can be prepared by combining a dual binding antibody or a dual binding antibody-containing composition with an appropriate physiologically acceptable carrier, diluent or excipient, and may be formulated into preparations in solid, semi-solid, liquid or gaseous forms, such as tablets, capsules, powders, granules, ointments, solutions, suppositories, injections, inhalants, gels, microspheres, and aerosols. In addition, other pharmaceutically active ingredients and/or suitable excipients such as salts, buffers and stabilizers may, but need not, be present within the composition. Administration may be achieved by a variety of different routes, including oral, parenteral, nasal, intravenous, intradermal, subcutaneous or topical. In some embodiments, modes of administration depend upon the nature of the condition to be treated or prevented. An amount that, following administration, reduces, inhibits, prevents or delays the progression and/or metastasis of a cancer is considered effective. A skilled artisan would appreciate that the term “physiologically acceptable carrier, diluent or excipient”, may in some embodiments be used interchangeably with the term “pharmaceutically acceptable carrier” having all the same means and qualities.

In some embodiments, a pharmaceutical composition described herein comprises a nucleotide sequence encoding a dual binding antibody. In some embodiments, a nucleotide sequence encoding a dual binding antibody disclosed herein, comprises a single linear nucleotide sequence. In some embodiments, a nucleotide sequence encoding a dual binding antibody disclosed herein, comprises two nucleotide sequences. In some embodiments, a nucleotide sequence encoding a dual binding antibody disclosed herein, comprises two nucleotide sequences present on the same vector. In some embodiments, a nucleotide sequence encoding a dual binding antibody disclosed herein, comprises two nucleotide sequences present on different vectors.

In some embodiments, the nucleotide sequence encodes a variant VH or a variant VL domain or a combination thereof. In some embodiments, the same nucleotide sequence encodes a variant VH or a variant VL domain or a combination thereof. In some embodiments, different nucleotide sequences encode a variant VH or a variant VL domain or a combination thereof. In some embodiments, one nucleotide sequence encodes a variant VH domain and another nucleotide sequence encodes a variant VL domain. In some embodiments, one nucleotide sequence encodes variant VH domain and another nucleotide sequence encodes a variant VL domain having a linker sequence between them, thus allowing a variant VH and a variant VL domain to hetero-dimerize, as described in Duperret E K et al., Cancer Res, October 4 (doi: 10.1158/0008-5472.CAN-18-1429).

In some embodiments, a method of treating an allergic or respiratory condition in a subject, or a combination thereof, comprises a step of administering a pharmaceutical composition comprising a dual binding antibody comprising (a) a variant VH domain comprising the amino acid sequence set forth in SEQ ID NO: 1 with at least one amino acid variant at any of positions 52, 99, 100, 101, 102, 103, 104, 105, 106, 107, 108, or 111, or any combination thereof (IMGT positions: 57, 107, 108, 109, 110, 111, 111A, 112A, 112, 113, 114, or 117, or a combination thereof); and (b) a variant VL domain comprising the amino acid sequence set forth in SEQ ID NO: 2 with at least one amino acid variant at any of positions 26, 27, 31, 51, 56, 77, 92, 93, or 96, or any combination thereof (IMGT positions: 27, 28, 38, 65, 70, 94, 109, 110, or 115, or a combination thereof); to a subject in need, wherein the method treats the allergic or respiratory condition, or a combination thereof, compared with a subject not administered said pharmaceutical composition.

In some embodiments, a method of treating an allergic or respiratory condition in a subject, or a combination thereof, comprises a step of administering a pharmaceutical composition comprising a dual binding antibody comprising (a variant VH domain comprising the amino acid sequence set forth in SEQ ID NO: 1 with at least one amino acid variant at any of positions 52, 99, 100, 101, 102, 103, 104, 105, 106, 107, 108, or 111, or any combination thereof (IMGT positions: 57, 107, 108, 109, 110, 111, 111A, 112A, 112, 113, 114, or 117, or a combination thereof); to a subject in need, wherein the method treats the allergic or respiratory condition, or a combination thereof, compared with a subject not administered said pharmaceutical composition.

In some embodiments, a method of treating an allergic or respiratory condition in a subject, or a combination thereof, comprises a step of administering a pharmaceutical composition comprising a dual binding antibody comprising a variant VL domain comprising the amino acid sequence set forth in SEQ ID NO: 2 with at least one amino acid variant at any of positions 26, 27, 31, 51, 56, 77, 92, 93, or 96, or any combination thereof (IMGT positions: 27, 28, 38, 65, 70, 94, 109, 110, or 115, or a combination thereof); to a subject in need, wherein the method treats the allergic or respiratory condition, or a combination thereof, compared with a subject not administered said pharmaceutical composition.

### Methods of Use

In some embodiments, disclosed herein is a method of treating a subject suffering from a disease or condition, said method comprises administering to said subject a composition comprising an isolated dual binding antibody as disclosed herein. In some embodiments, the disease or condition is an allergic or respiratory condition, an inflammatory or autoimmune condition, or tumors or cancers. In some embodiments, said disease or condition is asthma, allergic asthma, nonallergic asthma, severe asthma, mild asthma, chronic obstructive pulmonary disease (COPD), a condition involving airway inflammation, cystic fibrosis, allergic lung disease, airway hyperresponsiveness, goblet cell metaplasia, mucus hypersecretion, airway remodeling, pulmonary fibrosis, atopic dermatitis, urticaria, eczema, allergic enterogastritis, allergic rhinitis, inflammatory bowel diseases, liver cirrhosis or fibrosis, or a combination thereof.

In some embodiments, a method of treating an allergic or respiratory condition, an inflammatory and/or autoimmune condition of the skin or gastrointestinal organs; scleroderma; or tumors or cancers including Hodgkin's lymphoma in a subject, or any combination thereof, comprises a step of administering a pharmaceutical composition comprising a dual binding antibody or a pharmaceutical composition thereof, said dual binding antibody comprising a heavy chain variable region comprising HCDRs (HCDR1, HCDR2, HCDR3 as described herein in detail; for example, see Table 8 or Table 4). In some embodiments, a method of treating an allergic or respiratory condition, an inflammatory and/or autoimmune condition of the skin or gastrointestinal organs; scleroderma; or tumors or cancers including Hodgkin's lymphoma in a subject, or any combination thereof, comprises a step of administering a pharmaceutical composition comprising a dual binding antibody or a pharmaceutical composition thereof, said dual binding antibody comprising a light chain variable region comprising LCDRs (LCDR1, LCDR2, LCDR3 as described herein in detail; for example, see Table 9 or Table 5). In some embodiments, a method of treating an allergic or respiratory condition, an inflammatory and/or autoimmune condition of the skin or gastrointestinal organs; scleroderma; or tumors or cancers including Hodgkin's lymphoma in a subject, or any combination thereof, comprises a step of administering a pharmaceutical composition comprising a dual binding antibody or a pharmaceutical composition thereof, said dual binding antibody comprising a heavy chain variable region comprising HCDRs (HCDR1, HCDR2, HCDR3) and LCDRs (LCDR1, LCDR2, LCDR3 as described herein in detail).

In certain embodiments, a method of treating a subject suffering from a disease or condition comprises administering a dual binding antibody comprising three complementarity determining regions (CDRs) on a heavy chain (HCDR1, HCDR2, and HCDR3) and three CDRs on a light chain (LCDR1, LCDR2, and LCDR3), wherein the HCDR1, HCDR2 and HCDR3 comprise the amino acid sequence of SEQ ID NOs:349, 350 and 351 respectively, and the LCDR1, LCDR2 and LCDR3 comprise the amino acid sequence of SEQ ID NO: 359, D D V, and SEQ ID NO: 361, respectively; or the HCDR1, HCDR2 and HCDR3 comprise the amino acid sequence of SEQ ID NOs:349, 356 and 351 respectively, and the LCDR1, LCDR2 and LCDR3 comprise the amino acid sequence of SEQ ID NO: 364, D D V, and SEQ ID NO: 371, respectively; or the HCDR1, HCDR2 and HCDR3 comprise the amino acid sequence of SEQ ID NOs:349, 350 and 351 respectively, and the LCDR1, LCDR2 and LCDR3 comprise the amino acid sequence of SEQ ID NO: 362, D D V, and SEQ ID NO: 384, respectively; or the HCDR1, HCDR2 and HCDR3 comprise the amino acid sequence of SEQ ID NOs:349, 350 and 351 respectively, and the LCDR1, LCDR2 and LCDR3 comprise the amino acid sequence of SEQ ID NO: 364, D D V, and SEQ ID NO: 384, respectively; or the CDRs having the sequences of SEQ ID NOs:149-152, D D S, and SEQ ID NO: 154.

In some embodiments a method of treating a subject suffering from a disease or condition comprises administering a dual binding antibody comprising three complementarity determining regions (CDRs) on a heavy chain (HCDR1, HCDR2, and HCDR3) and three CDRs on a light chain (LCDR1, LCDR2, and LCDR3), wherein the HCDR1, HCDR2 and HCDR3 comprise the amino acid sequences as shown in Table 8 or Table 4, wherein the LCDR1, LCDR2 and LCDR3 comprise the amino acid sequences as shown in Table 9 or Table 5.

In some embodiments a method of treating a subject suffering from a disease or condition comprises administering a dual binding antibody comprising VH and VL having the sequences of SEQ ID Nos:209 and 210, SEQ ID Nos:219 and 220, SEQ ID Nos:249 and 250, SEQ ID Nos:337 and 338, SEQ ID NOs:155 and 156, SEQ ID NOs:157 and 158. In some embodiments a method of treating a subject suffering from a disease or condition comprises administering a dual binding antibody comprising VH and VL domains having the sequences as shown in Table 10 or Table 1.

In some embodiments, a method of treating an allergic or respiratory condition, an inflammatory and/or autoimmune condition of the skin or gastrointestinal organs; scleroderma; or tumors or cancers including Hodgkin's lymphoma in a subject, or any combination thereof, comprises a step of administering a pharmaceutical composition comprising a dual binding antibody or a pharmaceutical composition thereof, said dual binding antibody comprising (a) a heavy chain variable region comprising the amino acid sequence set forth in SEQ ID NO: 1 with at least one amino acid variant at any of positions 52, 99, 100, 101, 102, 103, 104, 105, 106, 107, 108, or 111, or any combination thereof (IMGT positions: 57, 107, 108, 109, 110, 111, 111A, 112A, 112, 113, 114, or 117, or a combination thereof); (b) a light chain variable region comprising the amino acid sequence set forth in SEQ ID NO: 2 with at least one amino acid variant at any of positions 26, 27, 31, 51, 56, 77, 92, 93, or 96, or any combination thereof (IMGT positions: 27, 28, 38, 65, 70, 94, 109, 110, or 115, or a combination thereof); or a combination of the heavy chain variable region set forth in (a) and the light chain variable region set forth in (b); wherein the total number of variant positions in said heavy chain variable region, said light chain variable region, or said combination thereof, is at least 2, to a subject in need, wherein the method treats the allergic or respiratory condition, an inflammatory and/or autoimmune condition of the skin or gastrointestinal organs; scleroderma; or tumors or cancers including Hodgkin's lymphoma in said subject, compared with a subject not administered said dual binding antibody, or pharmaceutical composition thereof.

In some embodiments, a method of treating an allergic or respiratory condition, an inflammatory and/or autoimmune condition of the skin or gastrointestinal organs; scleroderma; or tumors or cancers including Hodgkin's lymphoma n in a subject, or any combination thereof, comprises a step of administering a pharmaceutical composition comprising a dual binding antibody or a pharmaceutical composition thereof, said dual binding antibody comprising a heavy chain variable region comprising the amino acid sequence set forth in SEQ ID NO: 1 with at least one amino acid variant at any of positions 52, 99, 100, 101, 102, 103, 104, 105, 106, 107, 108, or 111, or any combination thereof (IMGT positions: 57, 107, 108, 109, 110, 111, 111A, 112A, 112, 113, 114, or 117, or a combination thereof), wherein the total number of variant positions in said heavy chain variable region is at least 2, to a subject in need, wherein the method treats the allergic or respiratory condition, an inflammatory and/or autoimmune condition of the skin or gastrointestinal organs; scleroderma; or tumors or cancers including Hodgkin's lymphoma in said subject, compared with a subject not administered said dual binding antibody, or pharmaceutical composition thereof.

In some embodiments, a method of treating an allergic or respiratory condition, an inflammatory and/or autoimmune condition of the skin or gastrointestinal organs; scleroderma; or tumors or cancers including Hodgkin's lymphoma in a subject, or any combination thereof, comprises a step of administering a pharmaceutical composition comprising a dual binding antibody or a pharmaceutical composition thereof, said dual binding antibody comprising a light chain variable region comprising the amino acid sequence set forth in SEQ ID NO: 2 with at least one amino acid variant at any of positions 26, 27, 31, 51, 56, 77, 92, 93, or 96, or any combination thereof (IMGT positions: 27, 28, 38, 65, 70, 94, 109, 110, or 115, or a combination thereof), wherein the total number of variant positions in said light chain variable region is at least 2, to a subject in need, wherein the method treats the allergic or respiratory condition, an inflammatory and/or autoimmune condition of the skin or gastrointestinal organs; scleroderma; or tumors or cancers including Hodgkin's lymphoma in said subject, compared with a subject not administered said dual binding antibody, or pharmaceutical composition thereof.

In some embodiments, a method of treating an allergic or respiratory condition, an inflammatory and/or autoimmune condition of the skin or gastrointestinal organs; scleroderma; or tumors or cancers including Hodgkin's lymphoma in a subject, or any combination thereof, comprises a step of administering a pharmaceutical composition comprising a dual binding antibody or a pharmaceutical composition thereof, said dual binding antibody comprising (a) a heavy chain variable region comprising the amino acid sequence set forth in SEQ ID NO: 1 with at least one amino acid variant at any of positions 52, 99, 100, 101, 102, 103, 104, 105, 106, 107, 108, or 111, or any combination thereof (IMGT positions: 57, 107, 108, 109, 110, 111, 111A, 112A, 112, 113, 114, or 117, or a combination thereof); and (b) a light chain variable region comprising the amino acid sequence set forth in SEQ ID NO: 2 with at least one amino acid variant at any of positions 26, 27, 31, 51, 56, 77, 92, 93, or 96, or any combination thereof (IMGT positions: 27, 28, 38, 65, 70, 94, 109, 110, or 115, or a combination thereof); wherein the total number of variant positions in said heavy chain variable region, said light chain variable region, or said combination thereof of is at least 2, to a subject in need, wherein the method treats the allergic or respiratory condition, an inflammatory and/or autoimmune condition of the skin or gastrointestinal organs; scleroderma; or tumors or cancers including Hodgkin's lymphoma in said subject, compared with a subject not administered said dual binding antibody, or pharmaceutical composition thereof.

In some embodiments of a method of treating an allergic or respiratory condition, an inflammatory and/or autoimmune condition of the skin or gastrointestinal organs; scleroderma; or tumors or cancers including Hodgkin's lymphoma, the amino acid sequence of the variant VH domain is selected from, but not limited to, the sequences set forth in any of SEQ ID NOs: 4, 6, 8, 10, 12, 14, 16, 18, 20, 22, 24, 26, 28, 30, 32, 34, 36, 38, 40, 42, 44, 46, 48, 50, 52, and 54. In some embodiments of a method of treating an allergic or respiratory condition, an inflammatory and/or autoimmune condition of the skin or gastrointestinal organs; scleroderma; or tumors or cancers including Hodgkin's lymphoma, a dual binding antibody comprises a heavy chain variable region comprising the amino acid sequences set forth in, but not limited to, any of SEQ ID NOs: 4, 6, 8, 10, 12, 14, 16, 18, 20, 22, 24, 26, 28, 30, 32, 34, 36, 38, 40, 42, 44, 46, 48, 50, 52, and 54; and any variable light chain region. In some embodiments of a method disclosed herein, the amino acid sequence of the variant VH domain comprises the sequence that is at least 80% identical (e.g., 80%, 85%, 90%, 95%, 98%, or 99% identical) to the sequences set forth in any of SEQ ID NOs: 4, 6, 8, 10, 12, 14, 16, 18, 20, 22, 24, 26, 28, 30, 32, 34, 36, 38, 40, 42, 44, 46, 48, 50, 52, and 54.

In some embodiments of method disclosed herein, the VH domain of the dual binding antibody is selected from the sequences set forth in any one of SEQ ID NOs:209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345 and 347. In another embodiment, the VH domain is at least 80%, 85%, 90%, 95%, 98%, or 99% identical to the VH sequences disclosed herein.

One skilled in the art would appreciate that percent sequence identity may be determined using any of a number of publicly available software application, for example but not limited to BlastP software of the National Center of Biotechnology Information (NCBI) using default parameters.

In some embodiments of a method of treating an allergic or respiratory condition, an inflammatory and/or autoimmune condition of the skin or gastrointestinal organs; scleroderma; or tumors or cancers including Hodgkin's lymphoma, the amino acid sequence of the variant light chain variable region (VL) is selected from, but not limited to, the sequences set forth in any of SEQ ID NOs: 3, 5, 7, 9, 11, 13, 15, 17, 19 21, 23, 25, 27, 29, 31, 33, 35, 37, 39, 41, 43, 45, 47, 49, 51, and 53. In some embodiments of a method of treating an allergic or respiratory condition, an inflammatory and/or autoimmune condition of the skin or gastrointestinal organs; scleroderma; or tumors or cancers including Hodgkin's lymphoma, a dual binding antibody comprises a light chain variable region comprising the amino acid sequences set forth in, but not limited to, any of SEQ ID NOs: 3, 5, 7, 9, 11, 13, 15, 17, 19 21, 23, 25, 27, 29, 31, 33, 35, 37, 39, 41, 43, 45, 47, 49, 51, and 53, and any variable heavy chain region. In some embodiments of a method disclosed herein, the amino acid sequence of the variant VH domain comprises the sequence that is at least 80% identical (e.g., 80%, 85%, 90%, 95%, 98%, or 99% identical) to the sequences set forth in any of SEQ ID NOs: 3, 5, 7, 9, 11, 13, 15, 17, 19 21, 23, 25, 27, 29, 31, 33, 35, 37, 39, 41, 43, 45, 47, 49, 51, and 53, and any variable heavy chain region.

In some embodiments of methods disclosed herein, the VL domain of the dual binding antibody is selected from the sequences set forth in any one of SEQ ID NOs: 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346 and 348. In another embodiment, the VL domain is at least 80%, 85%, 90%, 95%, 98%, or 99% identical to the VL sequences disclosed herein.

In some embodiments of a method of treating an allergic or respiratory condition, an inflammatory and/or autoimmune condition of the skin or gastrointestinal organs; scleroderma; or tumors or cancers including Hodgkin's lymphoma, the amino acid sequence of the variant VH domain is selected from, but not limited to, the sequences set forth in any of SEQ ID NOs: 4, 6, 8, 10, 12, 14, 16, 18, 20, 22, 24, 26, 28, 30, 32, 34, 36, 38, 40, 42, 44, 46, 48, 50, 52, and 54; and the amino acid sequence of the variant light chain variable region (VH) is selected from, but not limited to, the sequences set forth in any of SEQ ID NOs: 3, 5, 7, 9, 11, 13, 15, 17, 19 21, 23, 25, 27, 29, 31, 33, 35, 37, 39, 41, 43, 45, 47, 49, 51, and 53. In some embodiments of a method of treating an allergic or respiratory condition, an inflammatory and/or autoimmune condition of the skin or gastrointestinal organs; scleroderma; or tumors or cancers including Hodgkin's lymphoma, the amino acid sequences of a heavy chain variable region-light chain variable region pair are selected from, but not limited to, the pair sequences set forth in SEQ ID Nos: 4 and 3, SEQ ID Nos: 6 and 5, SEQ ID Nos: 8 and 7, SEQ ID Nos: 10 and 9, SEQ ID Nos: 12 and 11, SEQ ID Nos: 14 and 13, SEQ ID Nos: 16 and 15, SEQ ID Nos: 18 and 17, SEQ ID Nos: 20 and 19, SEQ ID Nos: 22 and 21, SEQ ID Nos: 24 and 23, SEQ ID Nos: 26 and 25, SEQ ID Nos: 28 and 27, SEQ ID Nos: 30 and 29, SEQ ID Nos: 32 and 31, SEQ ID Nos: 34 and 33, SEQ ID Nos: 36 and 35, SEQ ID Nos: 38 and 37, SEQ ID Nos: 40 and 39, SEQ ID Nos: 42 and 41, SEQ ID Nos: 44 and 43, SEQ ID Nos: 46 and 45, SEQ ID Nos: 48 and 47, SEQ ID Nos: 50 and 49, SEQ ID Nos: 52 and 51, and SEQ ID Nos: 54 and 53. In some embodiments, the amino acid sequences of the VH-VL pair are selected from the pair sequences set forth in any one of the following: SEQ ID NOs:209 and 210, SEQ ID NOs:211 and 212, SEQ ID NOs:213 and 214, SEQ ID NOs:215 and 216, SEQ ID NOs:217 and 218, SEQ ID NOs:219 and 220, SEQ ID NOs:221 and 222, SEQ ID NOs:223 and 224, SEQ ID NOs:225 and 226, SEQ ID NOs:227 and 228, SEQ ID NOs:229 and 230, SEQ ID NOs:231 and 232, SEQ ID NOs:233 and 234, SEQ ID NOs:235 and 236, SEQ ID NOs:237 and 238, SEQ ID NOs:239 and 240, SEQ ID NOs:241 and 242, SEQ ID NOs:243 and 244, SEQ ID NOs:245 and 246, SEQ ID NOs:247 and 248, SEQ ID NOs:249 and 250, SEQ ID NOs:251 and 252, SEQ ID NOs:253 and 254, SEQ ID NOs:255 and 256, SEQ ID NOs:257 and 258, SEQ ID NOs:259 and 260, SEQ ID NOs:261 and 262, SEQ ID NOs:263 and 264, SEQ ID NOs:265 and 266, SEQ ID NOs:267 and 268, SEQ ID NOs:269 and 270, SEQ ID NOs:271 and 272, SEQ ID NOs:273 and 274, SEQ ID NOs:275 and 276, SEQ ID NOs:277 and 278, SEQ ID NOs:279 and 280, SEQ ID NOs:281 and 282, SEQ ID NOs:283 and 284, SEQ ID NOs:285 and 286, SEQ ID NOs:287 and 288, SEQ ID NOs:289 and 290, SEQ ID NOs:291 and 292, SEQ ID NOs:293 and 294, SEQ ID NOs:295 and 296, SEQ ID NOs:297 and 298, SEQ ID NOs:299 and 300, SEQ ID NOs:301 and 302, SEQ ID NOs:303 and 304, SEQ ID NOs:305 and 306, SEQ ID NOs:307 and 308, SEQ ID NOs:309 and 310, SEQ ID NOs:311 and 312, SEQ ID NOs:313 and 314, SEQ ID NOs:315 and 316, SEQ ID NOs:317 and 318, SEQ ID NOs:319 and 320, SEQ ID NOs:321 and 322, SEQ ID NOs:323 and 324, SEQ ID NOs:325 and 326, SEQ ID NOs:327 and 328, SEQ ID NOs:329 and 330, SEQ ID NOs:331 and 332, SEQ ID NOs:333 and 334, SEQ ID NOs:335 and 336, SEQ ID NOs:337 and 338, SEQ ID NOs:339 and 340, SEQ ID NOs:341 and 342, SEQ ID NOs:343 and 344, SEQ ID NOs:345 and 346, SEQ ID NOs:347 and 348.

In some embodiments, a method of treating one or more conditions in a subject as described herein comprises a step of administering a pharmaceutical composition comprising an isolated dual binding antibody comprising three complementarity determining regions (CDRs) on a heavy chain (HCDR1, HCDR2, and HCDR3) and three CDRs on a light chain (LCDR1, LCDR2, and LCDR3), wherein the CDRs have the sequences of SEQ ID NOs:149-154. In another embodiment, the dual binding antibody comprises a heavy chain variable domain (VH) and a light chain variable domain (VL) having the amino acid sequences of SEQ ID Nos:155 and 156, or SEQ ID Nos:157 and 158.

In some embodiments, a method of treating one or more conditions in a subject as described herein comprises a step of administering a pharmaceutical composition comprising an isolated dual binding antibody comprising three complementarity determining regions (CDRs) on a heavy chain (HCDR1, HCDR2, and HCDR3) and three CDRs on a light chain (LCDR1, LCDR2, and LCDR3), wherein the CDRs have the sequences set forth in SEQ ID NOs:349, 350 and 351, respectively, and SEQ ID NO: 359, D D V, and SEQ ID NO: 361, respectively.

In some embodiments, a method of treating one or more conditions in a subject as described herein comprises a step of administering a pharmaceutical composition comprising an isolated dual binding antibody comprising three complementarity determining regions (CDRs) on a heavy chain (HCDR1, HCDR2, and HCDR3) and three CDRs on a light chain (LCDR1, LCDR2, and LCDR3), wherein the CDRs have the sequences set forth in SEQ ID NOs:349, 356, and 351, respectively, and SEQ ID NO: 364, D D V, and SEQ ID NO: 371, respectively.

In some embodiments, a method of treating one or more conditions in a subject as described herein comprises a step of administering a pharmaceutical composition comprising an isolated dual binding antibody comprising three complementarity determining regions (CDRs) on a heavy chain (HCDR1, HCDR2, and HCDR3) and three CDRs on a light chain (LCDR1, LCDR2, and LCDR3), wherein the CDRs have the sequences set forth in SEQ ID NOs:349, 350, and 351, respectively, and SEQ ID NO: 362, D D V, and SEQ ID NO: 384, respectively.

In some embodiments, a method of treating one or more conditions in a subject as described herein comprises a step of administering a pharmaceutical composition comprising an isolated dual binding antibody comprising three complementarity determining regions (CDRs) on a heavy chain (HCDR1, HCDR2, and HCDR3) and three CDRs on a light chain (LCDR1, LCDR2, and LCDR3), wherein the CDRs have the sequences set forth in SEQ ID NOs:349, 350, and 351, respectively, and SEQ ID NO: 364, D D V, and SEQ ID NO: 384, respectively.

In some embodiments, a method of treating one or more conditions in a subject as described herein comprises a step of administering a pharmaceutical composition comprising an isolated dual binding antibody comprising three complementarity determining regions (CDRs) on a heavy chain (HCDR1, HCDR2, and HCDR3) and three CDRs on a light chain (LCDR1, LCDR2, and LCDR3), wherein the CDRs have the sequences set forth in Table 8 or Table 4, and Table 9 or Table 5.

In some embodiments, a method of treating one or more conditions in a subject as described herein comprises a step of administering a pharmaceutical composition comprising an isolated dual binding antibody comprising a heavy chain variable domain (VH) and a light chain variable domain (VL), wherein the VH and VL comprise the amino acid sequences of SEQ ID Nos:209 and 210.

In some embodiments, a method of treating one or more conditions in a subject as described herein comprises a step of administering a pharmaceutical composition comprising an isolated dual binding antibody comprising the amino acid sequences of SEQ ID Nos:219 and 220.

In some embodiments, a method of treating one or more conditions in a subject as described herein comprises a step of administering a pharmaceutical composition comprising an isolated dual binding antibody comprising amino acid sequences of SEQ ID Nos:249 and 250.

In some embodiments, a method of treating one or more conditions in a subject as described herein comprises a step of administering a pharmaceutical composition comprising an isolated dual binding antibody comprising the amino acid sequences of SEQ ID Nos:337 and 338.

In some embodiments, a method of treating one or more conditions in a subject as described herein comprises a step of administering a pharmaceutical composition comprising an isolated dual binding antibody comprising the amino acid sequences as shown in Table 10 or Table 1.

In some embodiments, a method of treating one or more conditions in a subject as described herein comprises a step of administering a pharmaceutical composition comprising an isolated dual binding antibody, wherein the VH and VL are at least 80%, 85%, 90%, 95%, 98%, or 99% identical to the VH and VL sequences disclosed herein.

The variant VH and VL domains have been described in detail above, including methods of creating these variant VH and VL domains by re-epitoping template sequence. That disclosure is incorporated herein in full, wherein methods of treating an allergic or respiratory condition, an inflammatory and/or autoimmune condition of the skin or gastrointestinal organs; scleroderma; or tumors or cancers including Hodgkin's lymphoma comprise use of any variant VH or VL domain described herein.

A skilled artisan would appreciate that the term “treating” and grammatical forms thereof, may in some embodiments encompass both therapeutic treatment and prophylactic or preventative measures with respect to an allergic or respiratory condition, as described herein, wherein the object is to treat, prevent, reduce, or alleviate, the allergic or respiratory condition, or symptoms thereof, or a combination thereof. Thus, in some embodiments of methods disclosed herein, treating may include directly affecting or curing, suppressing, inhibiting, preventing, reducing the severity of, delaying the onset of, reducing symptoms associated with the disease, disorder or condition, or a combination thereof; for example, when said disease or disorder comprises an allergic or respiratory condition. In some embodiments, “treating” encompasses enhancing the ability of host immune cells to destroy intracellular pathogens. In some embodiments, “treating” encompasses interference with the IL-13receptor/IL-4receptor signaling cascade. In some embodiments, “treating” encompasses inhibition of IL-13 activities. In some embodiments, “treating” encompasses reduction of IL-13 activities.

In some embodiments, “preventing” encompasses delaying the onset of symptoms or an allergic or respiratory condition. In some embodiments, “suppressing” or “inhibiting”, encompass reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, or a combination thereof.

In one embodiment, the subject is a mammal, e.g., a human suffering from one or more IL-13-associated disorders, including but not limited to respiratory disorders or conditions (e.g., asthma (e.g., allergic and nonallergic asthma for example but not limited to asthma due to infection with, e.g., respiratory syncytial virus (RSV), e.g., in younger children), severe asthma, mild asthma), chronic obstructive pulmonary disease (COPD), and other conditions involving airway inflammation, eosinophilia, fibrosis and excess mucus production (e.g., cystic fibrosis and pulmonary fibrosis); atopic disorders (e.g., atopic dermatitis, urticaria, eczema, allergic enterogastritis, and allergic rhinitis); inflammatory and/or autoimmune conditions of, the skin, gastrointestinal organs (e.g., inflammatory bowel diseases (IBD), such as ulcerative colitis and/or Crohn's disease), and liver (e.g., cirrhosis, fibrosis); scleroderma; or tumors or cancers, e.g., Hodgkin's lymphoma.

In some embodiments, methods of treating comprising treating, reducing, preventing, or ameliorating, symptoms of an allergic or respiratory condition, an inflammatory and/or autoimmune condition of the skin or gastrointestinal organs; scleroderma; or tumors or cancers including Hodgkin's lymphoma, or any combination thereof. For example, symptoms of asthma may include, but are not limited to, wheezing, shortness of breath, bronchoconstriction, airway hyperreactivity, decreased lung capacity, fibrosis, airway inflammation, and mucus production. The method comprises administering to the subject a dual binding antibody or a pharmaceutical composition thereof, as described herein, in an amount sufficient to treat (e.g., reduce, ameliorate) or prevent one or more symptoms. The dual binding antibody can be administered therapeutically or prophylactically, or both. The dual binding antibody can be administered to the subject, alone or in combination with other therapeutic modalities.

The precise dosage and duration of treatment is a function of the disease being treated and may be determined empirically using known testing protocols or by testing the compositions in model systems known in the art and extrapolating therefrom. Controlled clinical trials may also be performed. Dosages may also vary with the severity of the condition to be alleviated. A pharmaceutical composition is generally formulated and administered to exert a therapeutically useful effect while minimizing undesirable side effects. The composition may be administered one time or may be divided into a number of smaller doses to be administered at intervals of time. For any particular subject, specific dosage regimens may be adjusted over time according to the individual need.

In some embodiments, compositions comprising a nucleic acid construct, comprising a nucleic acid sequence encoding a dual binding antibody comprising: (a) a heavy chain variable region comprising the amino acid sequence set forth in SEQ ID NO: 1 with at least one amino acid variant at any of positions 52, 99, 100, 101, 102, 103, 104, 105, 106, 107, 108, or 111, or any combination thereof (IMGT positions: 57, 107, 108, 109, 110, 111, 111A, 112A, 112, 113, 114, or 117, or a combination thereof); (b) a light chain variable region comprising the amino acid sequence set forth in SEQ ID NO: 2 with at least one amino acid variant at any of positions 26, 27, 31, 51, 56, 77, 92, 93, or 96, or any combination thereof (IMGT positions: 27, 28, 38, 65, 70, 94, 109, 110, or 115, or a combination thereof); or (c) a combination of heavy chain variable region set forth in (a) and the light chain variable region set forth in (b); wherein the total number of variant positions in the encoded heavy chain variable region, the encoded light chain variable region, or a combination thereof, is at least 2, may be administered alone or in combination with other known allergic or respiratory condition, an inflammatory and/or autoimmune condition of the skin or gastrointestinal organs; scleroderma; or tumors or cancers including Hodgkin's lymphoma treatments.

In some embodiments, methods of treatment comprise administration of a composition comprising a nucleic acid construct comprising a dual binding antibody comprising an VH domain comprising HCDRs (HCDR1, HCDR2, HCDR3 as described herein; for example, see Table 8 or Table 4). In some embodiments, methods of treatment comprise administration of a composition comprising a nucleic acid construct comprising a dual binding antibody comprising an VL domain comprising LCDRs (LCDR1, LCDR2, LCDR3 as described herein; for example, see Table 9 or Table 5). In some embodiments, methods of treatment comprise administration of a composition comprising a nucleic acid construct comprising a dual binding antibody comprising an VH domain comprising HCDRs (HCDR1, HCDR2, HCDR3 as described herein) and a VL domain comprising LCDRs (LCDR1, LCDR2, LCDR3 as described herein).

In some embodiments, compositions comprising a nucleic acid construct, comprising a nucleic acid sequence encoding a dual binding antibody comprising: (a) a heavy chain variable region comprising the amino acid sequence set forth in SEQ ID NO: 1 with at least one amino acid variant at any of positions 52, 99, 100, 101, 102, 103, 104, 105, 106, 107, 108, or 111, or any combination thereof (IMGT positions: 57, 107, 108, 109, 110, 111, 111A, 112A, 112, 113, 114, or 117, or a combination thereof); wherein the total number of variant positions in the encoded heavy chain variable region is at least 2, may be administered alone or in combination with other known allergic or respiratory condition treatments. In some embodiments, compositions comprising a nucleic acid construct, comprising a nucleic acid sequence encoding a dual binding antibody comprising: a light chain variable region comprising the amino acid sequence set forth in SEQ ID NO: 2 with at least one amino acid variant at any of positions 26, 27, 31, 51, 56, 77, 92, 93, or 96, or any combination thereof (IMGT positions: 27, 28, 38, 65, 70, 94, 109, 110, or 115, or a combination thereof); wherein the total number of variant positions in the encoded light chain variable region is at least 2, may be administered alone or in combination with other known allergic or respiratory condition treatments. In some embodiments, compositions comprising a nucleic acid construct, comprising a nucleic acid sequence encoding a dual binding antibody comprising: (a) a heavy chain variable region comprising the amino acid sequence set forth in SEQ ID NO: 1 with at least one amino acid variant at any of positions 52, 99, 100, 101, 102, 103, 104, 105, 106, 107, 108, or 111, or any combination thereof (IMGT positions: 57, 107, 108, 109, 110, 111, 111A, 112A, 112, 113, 114, or 117, or a combination thereof); and (b) a light chain variable region comprising the amino acid sequence set forth in SEQ ID NO: 2 with at least one amino acid variant at any of positions 26, 27, 31, 51, 56, 77, 92, 93, or 96, or any combination thereof (IMGT positions: 27, 28, 38, 65, 70, 94, 109, 110, or 115, or a combination thereof); wherein the total number of variant positions in the encoded heavy chain variable region, the encoded light chain variable region, or a combination thereof, is at least 2, may be administered alone or in combination with other known allergic or respiratory condition treatments.

In some embodiments of a method of treating an allergic or respiratory condition, the nucleotide construct encoding the variant VH domain is selected from the sequences set forth in any of SEQ ID NOs: 57, 59, 61, 63, 65, 67, 69, 71, 73, 75, 77, 79, 81, 83, 85, 87, 89, 91, 93, 95, 97, 99, 101, 105, and 107. In some embodiments of a method of treating an allergic or respiratory condition, the nucleotide construct encoding the variant light chain variable region (VH) is selected from the sequences set forth in any of SEQ ID NOs: 58, 60, 62, 64, 66, 68, 70, 72, 74, 76, 78, 80, 82, 84, 86, 88, 90, 92, 94, 96, 98, 100, 102, 104, 106, and 108. In some embodiments of a method of treating an allergic or respiratory condition, the nucleotide construct encoding the variant VH domain is selected from the sequences set forth in any of SEQ ID NOs: 57, 59, 61, 63, 65, 67, 69, 71, 73, 75, 77, 79, 81, 83, 85, 87, 89, 91, 93, 95, 97, 99, 101, 105, and 107; and, the nucleotide construct encoding the variant light chain variable region (VH) is selected from the sequences set forth in any of SEQ ID NOs: 58, 60, 62, 64, 66, 68, 70, 72, 74, 76, 78, 80, 82, 84, 86, 88, 90, 92, 94, 96, 98, 100, 102, 104, 106, and 108. In some embodiments of a method of treating an allergic or respiratory condition, the nucleotide construct encoding a dual binding antibody heavy chain variable region-light chain variable region scFv, is selected from the sequences set forth in SEQ ID NOs: 109-135.

The nucleotide sequences encoding variant VH and VL domains have been described in detail above, including methods of creating these nucleotide sequences encoding the variant VH and VL domains by mutating template sequence. That disclosure is incorporated herein in full, wherein methods of treating an allergic or respiratory condition comprise use of any nucleic acid construct encoding variant VH or VL domain described herein.

In some embodiments of a method of treating an allergic or respiratory condition, a dual binding antibody described herein comprises an IgG immunoglobulin. In some embodiments of a method of treating an allergic or respiratory condition, a dual binding antibody comprises an IgG1 immunoglobulin, an IgG2 immunoglobulin, an IgG3 immunoglobulin, or an IgG4 immunoglobulin. In some embodiments of a method of treating an allergic or respiratory condition, a dual binding antibody comprises an IgG1 immunoglobulin. In some embodiments of a method of treating an allergic or respiratory condition, a dual binding antibody comprises an IgG2 immunoglobulin. In some embodiments, a dual binding antibody comprises an IgG3 immunoglobulin. In some embodiments of a method of treating an allergic or respiratory condition, a dual binding antibody comprises an IgG4 immunoglobulin. In some embodiments of a method of treating an allergic or respiratory condition, a dual binding antibody comprises an IgG1 immunoglobulin or an IgG4 immunoglobulin.

In some embodiments of a method of treating an allergic or respiratory condition, a dual binding antibody comprises a mutated IgG, said mutant IgG unable to bind antibody-dependent cellular cytotoxicity components. In some embodiments of a method of treating an allergic or respiratory condition, a dual binding antibody comprises a mutated IgG1, said mutant IgG1 unable to bind antibody-dependent cellular cytotoxicity components. In some embodiments of a method of treating an allergic or respiratory condition, a dual binding antibody comprises a mutated IgG4, said mutant IgG4 unable to bind antibody-dependent cellular cytotoxicity components.

In some embodiments of a method of treating an allergic or respiratory condition, a dual binding antibody comprises an Fab immunoglobulin fragment. In some embodiments of a method of treating an allergic or respiratory condition, a dual binding antibody comprises an F(ab′)₂ immunoglobulin fragment. In some embodiments of a method of treating an allergic or respiratory condition, a dual binding antibody comprises an Fv immunoglobulin construct. In some embodiments of a method of treating an allergic or respiratory condition, a dual binding antibody comprises an scFv immunoglobulin construct. In some embodiments of a method of treating an allergic or respiratory condition, a dual binding antibody comprises a minibody immunoglobulin construct comprising a pair of single-chain Fv fragments which are linked via CH3 domains.

In some embodiments of a method of treating an allergic or respiratory condition, a dual binding antibody comprises a diabody immunoglobulin construct. In some embodiments of a method of treating an allergic or respiratory condition, a dual binding antibody comprises a triabody immunoglobulin construct.

Typical routes of administering these and related dual binding antibodies or pharmaceutical compositions thereof, include, without limitation, oral, topical, transdermal, inhalation, parenteral, sublingual, buccal, rectal, vaginal, and intranasal. The term parenteral as used herein includes subcutaneous injections, intravenous, intramuscular, intrasternal injection or infusion techniques. Dual binding antibodies or pharmaceutical compositions thereof according to certain embodiments as described herein, are formulated so as to allow the active ingredients contained therein to be bioavailable upon administration of the composition to a patient. Dual binding antibodies or pharmaceutical compositions thereof that will be administered to a subject or patient may take the form of one or more dosage units, where for example, a tablet may be a single dosage unit, and a container of a herein described dual binding antibody in aerosol form may hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The dual binding antibodies or pharmaceutical compositions thereof to be administered will, in any event, contain a therapeutically effective amount of a dual binding antibody of the present disclosure, for treatment of an allergic or respiratory condition.

A pharmaceutical composition may be in the form of a solid or liquid. In some embodiments, the pharmaceutically acceptable carrier(s) are particulate, so that the compositions are, for example, in tablet or powder form. The pharmaceutically acceptable carrier(s) may be liquid, with the compositions being, for example, an oral oil, injectable liquid or an aerosol, which is useful in, for example, inhalatory administration. When intended for oral administration, the pharmaceutical composition is preferably in either solid or liquid form, where semi-solid, semi-liquid, suspension and gel forms are included within the forms considered herein as either solid or liquid.

As a solid composition for oral administration, the pharmaceutical composition may be formulated into a powder, granule, compressed tablet, pill, capsule, chewing gum, wafer or the like. Such a solid composition will typically contain one or more inert diluents or edible pharmaceutically acceptable carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent. When the pharmaceutical composition is in the form of a capsule, for example, a gelatin capsule, it may contain, in addition to materials of the above type, a liquid pharmaceutically acceptable carrier such as polyethylene glycol or oil.

The pharmaceutical composition may be in the form of a liquid, for example, an elixir, syrup, solution, emulsion or suspension. The liquid may be for oral administration or for delivery by injection, as two examples. When intended for oral administration, preferred composition contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

The liquid pharmaceutical compositions, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid pharmaceutical composition intended for either parenteral or oral administration should contain an amount of a dual binding antibody as herein disclosed such that a suitable dosage will be obtained.

The pharmaceutical composition may be intended for topical administration, in which case the pharmaceutically acceptable carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a pharmaceutical composition for topical administration. If intended for transdermal administration, the composition may include a transdermal patch or iontophoresis device. The pharmaceutical composition may be intended for rectal administration, in the form, for example, of a suppository, which will melt in the rectum and release the drug. The composition for rectal administration may contain an oleaginous base as a suitable nonirritating excipient. Such bases include, without limitation, lanolin, cocoa butter and polyethylene glycol.

The pharmaceutical composition may include various materials, which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredient (a dual binding antibody) may be encased in a gelatin capsule. The pharmaceutical composition in solid or liquid form may include an agent that binds to the antibody as disclosed herein, and thereby assists in the delivery of the compound. Suitable agents that may act in this capacity include other monoclonal or polyclonal antibodies, one or more proteins or a liposome. The pharmaceutical composition may consist essentially of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols may be delivered in single phase, bi-phasic, or tri-phasic systems in order to deliver the active ingredient(s). Delivery of the aerosol includes the necessary container, activators, valves, subcontainers, and the like, which together may form a kit. One of ordinary skill in the art, without undue experimentation may determine preferred aerosols.

The pharmaceutical compositions may be prepared by methodology well known in the pharmaceutical art. For example, a pharmaceutical composition intended to be administered by injection can be prepared by combining a composition that comprises a dual binding antibody as described herein and optionally, one or more of salts, buffers and/or stabilizers, with sterile, distilled water so as to form a solution. A surfactant may be added to facilitate the formation of a homogeneous solution or suspension. Surfactants are compounds that non-covalently interact with the dual binding antibody composition so as to facilitate dissolution or homogeneous suspension of the dual binding antibody in the aqueous delivery system.

The compositions may be administered in a therapeutically effective amount, which will vary depending upon a variety of factors including the activity of the dual binding antibody employed; the metabolic stability and length of action of the dual binding antibody; the age, body weight, general health, sex, and diet of the patient; the mode and time of administration; the rate of excretion; the drug combination; the severity of the particular allergic or respiratory disorder or condition; and the subject undergoing therapy.

Compositions comprising the dual binding antibody of the present disclosure or comprising a nucleotide sequence encoding the dual binding antibody may also be administered simultaneously with, prior to, or after administration of one or more other therapeutic agents. Such combination therapy may include administration of a single pharmaceutical dosage formulation which contains a dual binding antibody as disclosed herein, and one or more additional active agents, as well as administration of compositions comprising dual binding antibody as disclosed herein, and each active agent in its own separate pharmaceutical dosage formulation. For example, a dual binding antibody or comprising a nucleotide sequence encoding the dual binding antibody, as described herein, and the other active agent can be administered to the patient together in a single oral dosage composition such as a tablet or capsule, or each agent administered in separate oral dosage formulations. Similarly, a dual binding antibody or comprising a nucleotide sequence encoding the dual binding antibody, as described herein, and the other active agent can be administered to the patient together in a single parenteral dosage composition such as in a saline solution or other physiologically acceptable solution, or each agent administered in separate parenteral dosage formulations. Where separate dosage formulations are used, the compositions comprising dual binding antibody or comprising a nucleotide sequence encoding the dual binding antibody, and one or more additional active agents can be administered at essentially the same time, i.e., concurrently, or at separately staggered times, i.e., sequentially and in any order; combination therapy is understood to include all these regimens.

Thus, in certain embodiments, also contemplated is the administration of dual binding antibody compositions of this disclosure or comprising a nucleotide sequence encoding the dual binding antibody, in combination with one or more other therapeutic agents. Such therapeutic agents may be accepted in the art as a standard treatment for a particular disease state as described herein, such as an allergic or respiratory condition.

In some embodiments, a pharmaceutically acceptable carrier may be liquid, semi-liquid or solid. Solutions or suspensions used for parenteral, intradermal, subcutaneous or topical application may include, for example, a sterile diluent (such as water), saline solution, fixed oil, polyethylene glycol, glycerin, propylene glycol or other synthetic solvent; antimicrobial agents (such as benzyl alcohol and methyl parabens, phenols or cresols, mercurials, chlorobutanol, methyl and propyl p-hydroxybenzoic acid esters, thimerosal, benzalkonium chloride and benzethonium chloride); antioxidants (such as ascorbic acid and sodium bisulfite; methionine, sodium thiosulfate, platinum, catalase, citric acid, cysteine, thioglycerol, thioglycolic acid, thiosorbitol, butylated hydroxyanisol, butylated hydroxytoluene, and/or propyl gallate) and chelating agents (such as ethylenediaminetetraacetic acid (EDTA)); buffers (such as acetates, citrates and phosphates). If administered intravenously, suitable pharmaceutically acceptable carriers include physiological saline or phosphate buffered saline (PBS), and solutions containing thickening and solubilizing agents, such as glucose, polyethylene glycol, polypropylene glycol and mixtures thereof.

The compositions comprising a dual binding antibody as described herein, may be prepared with pharmaceutically acceptable carriers that protect the dual binding antibody against rapid elimination from the body, such as time release formulations or coatings. Such pharmaceutically acceptable carriers include controlled release formulations, such as, but not limited to, implants and microencapsulated delivery systems, and biodegradable, biocompatible polymers, such as ethylene vinyl acetate, polyanhydrides, polyglycolic acid, polyorthoesters, polylactic acid and others known to those of ordinary skill in the art.

As used herein, the singular form “a”, “an” and “the” include plural references unless the context clearly dictates otherwise. For example, the term “an immunoglobulin” or “at least one immunoglobulin” may include a plurality of immunoglobulins, including mixtures thereof.

Throughout this application, various embodiments may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

### Examples

### Example 1: Experimental Procedures

Objective: To generate unique, dual binding antibodies.

Library Design Methods:

To generate a dual binding antibody that binds IL-13 and TSLP, a “re-epitoping” approach was applied to an existing antibody. The re-epitoping process allows for the introduction of new specificity to an existing antibody, e.g. a known antibody with 3-dimensional structure and well established biochemical and biophysical properties. The re-epitoped antibody is likely therefore to have both a new specificity and desirable developability properties. Briefly, re-epitoping is an engineering approach that allows the redirection of an existing antibody toward a new epitope, possibly on a new antigen unrelated to the cognate antigen of the original antibody. The computational process of re-epitoping requires two steps: (i) using any computational analysis that identifies putative contacts between an existing antibody and a new epitope, and (ii) application of any computational analysis or tool that can suggest the introduction of specific mutations into the antibody that are predicted to enhance its binding to the new, desired epitope. In (Ref: Ofran Y et al, US20180068055A1; Nimrod G, et al, Cell Rep. 2018 Nov. 20; 25(8):2121-2131) some examples of possible such computational processes are presented. In particular, two libraries were designed using the sequence of the variable domains of a template antibody (SEQ ID NO: 1-template variable heavy chain sequence; SEQ ID NO: 2—template variable light chain sequence)) as a starting point.

Template Variable Heavy Chain:
(SEQ ID NO: 1)
QMQLVESGGGVVQPGRSLRLSCAASGFTFRTYGMHWVRQAPGKGLEWV
AVIWYDGSNKHYADSVKGRFTITRDNSKNTLNLQMNSLRAEDTAVYYC
ARAPQWELVHEAFDIWGQGTMVTVSS.
Template Variable Light Chain:
(SEQ ID NO: 2)
SYVLTQPPSVSVAPGQTARITCGGNNLGSKSVHWYQQKPGQAPVLVVY
DDSDRPSWIPERFSGSNSGNTATLTISRGEAGDEADYYCQVWDSSSDH
VVFGGGTKLTVL.

Each library contained 21 positions that were chosen for variation with respect to the template original sequences. These positions are located in both CDRs (H2, H3, L1, L2, and L3) and framework (FIGS. 1A-1B). The following positions were chosen for variation in the libraries (IMGT® numbering scheme [the international ImMunoGeneTics information system® http://www.imgt.org): Variable H chain (SEQ ID NO: 1): 57(H2), 107(H3), 108(H3), 109(H3), 110(H3), 111(H3), 111A(H3), 112A(H3), 112(H3), 113(H3), 114(H3), 117(H3).

Variable L chain (SEQ ID NO: 2): 27(L1), 28(L1), 38 (P1(2), 65(L2), 70(1-R3), 94(FR3), 109(L3), 110(L3), 115(L3).

The resulting IL13/TSLP binding antibodies comprising variant heavy chain/variant light chain pairs, included a clone (C2) that contained 8 mutations relative to the template starting sequences (See, FIGS. 1A and 1B).

Library Construction Methods:

Libraries were constructed on the 5J13 template (PDB5J13) by overlapping extension PCR with degenerate oligonucleotides encoding the diversity 2*10\hat{ }14. PCR to introduce diversity was done using Phusion high fidelity DNA polymerase (New England Biolabs USA, Cat: M0530) according to manufacturer instructions in a 3-step reaction (98° C. for 30 sec, 65° C. for 20 sec, 72° C. for 30 sec, 30 cycles). The PCR products were gel purified by gel purification kit and assembled (100 ng from each) in equimolar ratios in a 3-step PCR reaction, as above, in the absence of primers. The assembled PCR product was reused as the template for PCR amplifying the full scFv library, as above, using forward and reverse primers adding vector sequences 5′ and 3′ to the scFv library to efficiently perform homologous recombination in yeast cells.

Library transformation was carried out as published (Benatuil et al., (2010) An improved yeast transformation method for the generation of very large human antibody libraries. Protein Eng. Des. Sel. 23, 155-159. 400 μl of a yeast suspension (EBY100, ATCC, USA) per 0.2 cm cuvette (cell projects) was electroporated (BioRad, USA, GenePulser) with 4 μg linearized vector (pCTcon3) and 12 μg DNA insert (scFv Library) in a 1:3 vector to insert ratio (Chao, G. et al. Isolating and engineering human antibodies using yeast surface display. Nat. Protoc. 1, 755-768 (2006)). The number of transformants of each library was determined to ˜1×10⁸ by serial dilutions of transformed cells (Benatuil et al. (2010) ibid)

Methods of Screening and Selection Using Yeast Surface Display:

Yeast-displayed scFv libraries were grown in a SDCAA selective medium and induced for expression with 2% w/v galactose at 30° C. overnight according to established protocols (Chao et al., (2006) ibid) The library was screened on BioRad S3e Fluorescence Activated Cell Sorter for high affinity binders of rh-IL-13-Fc (Reprokine, Israel) using mouse anti Myc-FITC (Santa Cruze, USA) and goat anti human Fc-APC (Jackson Immuno research, USA). Isolated clones from the final sort were sequenced by extraction of plasmid DNA from the yeast clones using a Zymoprep kit (Zymo Research, USA) and the DNA was sequenced. The chosen clones were incubated with either 10 nM recombinant human IL-13 (rh-IL-13)-Fc or 10 nM recombinant human TSLP (hTSLP)-Fc for 1 hour at room temperature. Cells were washed and resuspended in ice-cold PBS 0.1% BSA buffer containing a fluorescent labeled secondary antibody as described above for 20 min and analyzed using a flow cytometer. The values obtained were normalized to expression levels and to a positive control (an anti-IL-13 or anti TSLP binding antibody).

Methods of IgG-Roduction—Production of the IgGs Including the Light Chain (LC) and Heavy Chain (HC) Variable Regions:

Sequences of the selected clones were synthesized as GeneBlock (GB) with 5′ 25 bp region homologous to the cloning regions of pSF-CMV-HuIgG1_HC and pSF-CMV-HuLambda_LC (Oxford genetics, Oxford UK), the GB codon usage was optimized for mammalian expression (integrated DNA Technologies. Coralville, Iowa USA). The pSF-CMV-HuIgG1_HC and pSF-CMV-HuLambda_LC were digested using BseRI and NcoI, and the LC and HC variable region DNA fragments were cloned into the expression corresponding vectors using NEBuilder (NEB Ipswich, Massachusetts, USA). The expression vectors were transfected and expressed in ExpiCHO Expression System (ThermoFisher Scientific, USA) according to the manufacturer's instructions. Briefly: 25 ml CHO cells were grown at 37° C. to a density of 6*10\hat{ }6 cell/ml, 25 μg expression vector 1:2 HC/LC ratio were transfected into CHO cells, 20 hours post transfection the cells growth conditions were changed to 32° C. with 120 rpm shaking for 10 days. Subsequently the cells were centrifuged and IgGs were purified from the supernatant using proteinA beads, followed by size exclusion chromatography on a Superdex® 200 10/300 increase column (GE) with PBS serving as mobile phase.

Methods of Determination of IgG EC50 Binding to Human and Cynomolgus Monkey TSLP

Plates (Greiner Bio-One Cat: 655081) were coated with 45.5 ng/well human or cynomolgus monkey (cyno) TSLP antigen, then washed and blocked with 3% skim milk in PBS with 0.05% tween. Post blocking the tested IgG was added to the wells in a concentration range of 1 nM-1000 nM and incubated for 1 hour at room temperature (RT). The plates were washed and goat anti-human Fc-HRP conjugated secondary antibody (Jackson cat: 109-035-008) diluted 1:20000 in PBS, was added. The reaction was developed and stopped using TMB (Southern-Biotech cat: 0410-01) and stop solution (Southern-Biotech cat: 0412-01) respectively and read at 450 nm.

Methods of Determination of IC50 Competition Between IL-13 and TSLP

Plates were coated with ing/ul hTSLP washed with TBS 0.05% tween (TBS-T) and blocked with TBS-T 2% BSA. 20 nM of tested IgG was incubated with rhIL-13 at a concertation range of 0.78 nM to 200 nM for 1 hour, then the mixture was loaded on the plates for 10 minutes and the wells were washed and a bound IgG was detected using anti human Fc-HRP conjugate as described for the ELISA EC50 experiment above. A reciprocal competition experiment was conducted using the same conditions except this time IL-13 was coated on the wells, and TSLP served as free competing ligand at the same concentration range.

Methods of Determination of IgG IC50 Inhibition Constant of Blocking TSLP from Binding to TSLP-R:

150 ng/well of TSLP-R-Fc tag (ACRO biosystems TSR-H525a) was diluted in 0.015M NaHCO₃, pH=9.5, and was then used to coat the wells of a 96 well plate (Greiner Bio-One Cat: 655081). Wells were then washed three times with TBS 0.05% tween (TBS-T) and blocked with TBS-T containing 2% BSA (w/v). Competitor IgG at a concentration range of 0.11 nM to 300 nM was mixed with 3 nM hTSLP-His (ACRO biosystems cat: TSP-H52Hb) for one hour, then the mixture was loaded into the wells of the 96 well plate, incubated for 10 minutes, followed by washing the plate three times with TBS-T. Subsequently 1:200 anti-His-HRP conjugated secondary antibody was added (Santa Cruz Biothechnology cat SC-8036). The reaction was developed and stopped using TMB (Southern-Biotech cat: 0410-01) and stop solution (Southern-Biotech cat: 0412-01) respectively, and read at 450 nm.

Methods of Specificity Determination by ELISA

96 well plates (Greiner Bio-One Cat: 655081) were coated with a total of 250 ng ligand, blocked with PBS-T containing 0.5% (w/v) BSA, and incubated with 100 nM IgG. Plates were developed using the same reagents and conditions as in the TSLP EC50 experiment described herein.

Methods of Calibration of MUTZ5 TSLP Reporter Cell Line:

The in-vitro activity of anti-TSLP blockade of TSLP binding to its cognate receptor is based on detection of pSTAT5 activation by human TSLP in MUTZ5 human leukemia cell line (Francis et al., (2016) Hematopoiesis, 101(4):417-426). In order to determine the EC50 value of hTSLP STATS activation of MUTZ5 cell line, cells were inoculated in a total volume of 150 μl, 250×10⁵ cells/well and incubated for 1 hr at 37° C. 5% CO₂ in a 96 well plate. Then TSLP at concentration range of 0.1 pg/mml to 1000 pg/ml was added for 30 minutes. Subsequently cells were washed, blocked with Fc blocker (BD bioscience FC Blocker-MIX cat #BD564220), and fixated with cytofix fixation buffer (BD bioscience Cat #554655). The cells were permeabilized with 90% methanol, washed and labeled with anti-pSTAT5-PE (BD bioscience cat #562077). Treated MUTZ5 cells were analyzed for pSTST5 activation on a CytoFLEX S flow cytometer (Beckman). Cells gated for singlets and pSTAT5 were marked as pSTAT5 positive.

Methods of Determination of IgG IC50 Inhibition of MUTZ5 pSTAT5 Activation by hTSLP

To test functional blocking of pSTAT5 activation, IgG at a concentration range of 0.48 pM to 500 pM, was mixed with 14 pM hTSLP (ACROBiosystems, cat #TSP-H52Hb) and incubated for 30 minutes, then added to the cells for another 60 minutes. Subsequently the cells were washed, fixated, labeled, and analyzed as described for the calibration of MUTZ5 cells.

Methods of Surface Plasmon Resonance (SPR) Analysis

Measurements of IgG binding to human IL-13: The SPR analysis was done on Biacore 200 (GE) on CMS chips cat: br10005-30 (GE), the chip was crosslinked with primary capture Ab (Cat: br-1008-39 GE) to a target of 8000RU, after cross linking of the primary Ab, the tested antibodies were immobilized on the primary Ab to a target of 500RU. The hIL-13 (Peprotech) analyte was streamed in HEB-EP buffer at concentrations ranging from 800 nM to 1.6 nM in a series of two-fold dilutions, one concentration for each cycle. Subsequent to a cycle, the analyte and tested antibody were stripped from the chip and new tested Ab was loaded on the chip as described above. KD was determined at a steady state condition.

Measurements of Binding to Cynomolgus Monkey IL-13 (cIL-13, Sino Biological, USA) and Human TSLP

The SPR analysis was done on ProteOn™ XPR36 (BioRad) on a GLC chips cat: 176-5011 (BioRad). The chip was crosslinked with primary capture Ab (Cat: br-1008-39 GE) to a target of 5500RU. After cross-linking of the primary Ab tested, antibodies 33.003 and 33.004 were immobilized on the primary Ab to a target of 2000RU. The cyno IL-13 analyte was streamed in HEB-EP buffer at concentrations ranging from 200 nM to 12.5 nM in a series of two-fold dilutions. KD was determined at a steady state condition. For measurements of binding kinetics to hTSLP, the same conditions were used but with TSLP serving as analyte at concentrations ranging from 3.2 nM to 0.2 nM in a series of two-fold dilutions.

Method of Dynamic Scanning Fluorescence (DSF) Dynamic Scanning Fluorescence was measured as reported by (Niedziela-Majka et al., 2015) with minor modifications. Briefly: 0.3 mg/ml tested antibody in sodium acetate pH 5.5 buffer was mixed 1:1 with 20xsypro orange (Thermo Fisher, USA cat #56650) in the same buffer Changes in fluorescence were monitored on a Bio-Rad cfx96 light cycler with setting of 0.5° C./min from 25° C.-100° C. Tm was determined as the temperature corresponding to the maximum value of the first derivative of the DSF melting curve. Where mentioned, antibodies were diluted to 0.5 mg/ml in PBS and analyzed using NanoDSF Prometheus NT.48 (Nanotemper, Germany) in a temperature elevation rate of 1° C./min

Methods of Cell Based Assays

HEK-Blue IL-4/IL-13 Cells (Invivogen, France Catalog #hkb-i1413) were used to determine IL-13 inhibition. HEK-Blue cells were cultured in growth medium comprising of DMEM, 4.5 g/l glucose, 10% (v/v) fetal bovine serum (PBS), 50 U/ml penicillin, 50 mg/ml streptomycin, 100 mg/ml Normocin, 2 mM L-glutamine, 10 μg/ml of blasticidin and 100 pg/ml of Zeocin. HEK-Blue IL-4/IL-13 cells are specifically designed to monitor the activation of the STAT6 pathway induced by IL-4 and IL-13. These cells were generated by stably introducing the human STAT6 gene into HEK293 cells to obtain a fully active STAT6 signaling pathway. The other genes of the pathway are naturally expressed in sufficient amounts. HEK-BlueIL-4/dual cells stably express the reporter gene, secreted embryonic alkaline phosphatase (SEAP), under the control of the IFNβ minimal promoter fused to four STAT6 binding sites. Activation of the STAT6 pathway in HEK-Blue IL-4/IL-13 cells induces the expression of the reporter gene. SEAP, which is secreted in the supernatant is easily detectable when using QUANTI-Blue, a medium that turns purple/blue in the presence of SEAP.

Methods of Calibration of HEK-Blue IL-4/IL-13 System

In order to determine the EC₅₀ value for rh-IL-13 on HEK-Blue IL-4/IL-13 cells, 50000 cells (5*10\hat{ }4/ELISA well) were incubated with rh-IL-13 antibody (Peprotech, Israel) at concentration of 0 nM to 8.13 nM for 24 hrs at 37° C., 5% CO 2 in a 96 well plate. At the end of the incubation, 20 ul of the cell's supernatant was incubated with 180111 of QUANTI-Blue reagent (Invivogen, France) for an additional 2 hrs, and the reaction was analyzed by measuring the absorbance at 620-655 nm using a plate reader spectrophotometer (Synergy Neo2, BioTek Instruments, Inc. USA). Data shown is the mean of triplicate experiments, and error bars represent standard deviation.

IC₅₀ of Antibody Inhibition of IL-13 Downstream Signaling:

0.4 nM of rh-IL-13 was incubated with antibodies at a range of concentrations for 1 hr at room temperature. After the incubation, the mixture of rh-IL-13-antibody was added to a total volume of 200 μl, 50,000 cells/well and incubated for 24 hrs at 37 C 5% CO 2 in a 96 well plate. At the end of the incubation, 20 μl of the cell's supernatant was incubated with 180 ul of QUANTI-Blue reagent for additional 2 hrs, and the reaction was analyzed by measuring the absorbance at 620-655 nm using a plate reader spectrophotometer. Data shown is the mean of triplicate experiments, and error bars represent standard deviation.

### Example 2: Screening And Selection of Dual Binding Antibodies

Objective: Screen engineered dual binding antibodies to identify those with highest binding for IL-13 and TSLP.

Results: Following screening and selection of the libraries to bind both IL-13 and TSLP, 45 clones were selected, isolated, and sequenced resulting in 26 unique Heavy chain (VH)—Light chain (VL) pair variable regions, wherein the amino acid sequences of the Heavy chain and Light chain pairs are presented in Table 1 (antibodies 1-26), the nucleotide sequences of the Heavy chain and Light chain scFv for antibodies 1-26, including the encoded linker sequences, are presented in Table 2, and the nucleic acid sequences of the Heavy chain and Light chain pairs for antibodies 1-26, are presented in Table 3. The Clone ID number for antibodies 1-26, is provided as “C #”—of each “Name” provided, for example at row 2, Clone C2 variable region pair comprises C2 VL sequence SEQ ID NO: 3 and C2 VH sequence SEQ ID NO: 4.

Subsequently, an affinity maturation library was screened and additional dual binding antibody clones identified that showed YSD tight binding to both hIL-13 and hTSLP (Ab clone #s: 33.023, 33.025, 38.014, 38.015. 38.018, 38.019, 38.021, 38.026, 38.040). The amino acid sequences of the VH/VL regions of clones 33.023, 33.025, 38.014, 38.015. 38.018, 38.019, 38.021, 38.026, 38.040, are presented in Table 1 and the nucleotide sequences encoding the VH/VL regions are presented in Table 3. The CDR regions of VH/VL pairs from Ab clone #s: 33.023, 33.025, 38.014, 38.015. 38.018, 38.019, 38.021, 38.026, 38.040, are provided in Table 4 and Table 5 below. These clones were selected for IgG production.

TABLE 1
Engineered dual binding antibodies:
Variable Light chain (VL) and Variable Heavy chain (VH)
amino acid sequences (See also FIGS. 1A and 1B)

| | SEQ | | | SEQ | | |
|---|---|---|---|---|---|---|
| Ab | ID | | | ID | | |
| # | NO: | Name | VL Sequence | NO: | Name | VH Sequence |
| 1 | 3 | C2- | SYVLTQPPSVSVAP | 4 | C2- | QMQLVESGGGVVQPGRSLR |
| | | VL | GQTARITCGGNLIG | | VH | LSCAASGFTFRTYGMHWVR |
| | | | SKLVHWYQQKPGQ | | | QAPGKGLEWVAVIWYDGSN |
| | | | APVLVVYDDSDRP | | | KHYADSVKGRFTITRDNSKN |
| | | | SLIPERFSGSNSGNT | | | TLNLQMNSLRAEDTAVYYC |
| | | | ATLTISRVEAGDEA | | | ARAPQWELTAEAFDIWGQG |
| | | | DYYCQVWDSSSDG | | | TMVTVSS |
| | | | VVFGGGTKLTVL | | | |
| 2 | 5 | C27- | SYVLTQPPSVSVAP | 6 | C27- | QMQLVESGGGVVQPGRSLR |
| | | VL | GQTARITCGGNLIG | | VH | LSCAASGFTFRTYGMHWVR |
| | | | SKLVHWYQQKPGQ | | | QAPGKGLEWVAVIWYDGSN |
| | | | APVLVVYDDSDRP | | | KHYADSVKGRFTITRDNSKN |
| | | | SLIPERFSGSNSGNT | | | TLNLQMNSLRAEDTAVYYC |
| | | | ATLTISRVEAGDEA | | | ARAPQWELVAEAFDLWGQG |
| | | | DYYCQVWDSSSDG | | | TMVTVSS |
| | | | VVFGGGTKLTVL | | | |
| 3 | 7 | C19- | SYVLTQPPSVSVAP | 8 | C19- | QMQLVESGGGVVQPGRSLR |
| | | VL | GQTARITCGGNLIG | | VH | LSCAASGFTFRTYGMHWVR |
| | | | SKLVHWYQQKPGQ | | | QAPGKGLEWVAVIWYDGSN |
| | | | APVLVVYDDSDRP | | | KHYADSVKGRFTITRDNSKN |
| | | | SLIPERFSGSNSGNT | | | TLNLQMNSLRAEDTAVYYC |
| | | | ATLTISRVEAGDEA | | | ARAPQWQLVAEAFDIWGQG |
| | | | DYYCQVWDTSSDG | | | TMVTVSS |
| | | | VVFGGGTKLTVL | | | |
| 4 | 9 | C43- | SYVLTQPPSVSVAP | 10 | C43- | QMQLVESGGGVVQPGRSLR |
| | | VL | GQTARITCGGNLIG | | VH | LSCAASGFTFRTYGMHWVR |
| | | | SKLVHWYQQKPGQ | | | QAPGKGLEWVAVIWYDGSN |
| | | | APVLVVYDDSDRP | | | KHYADSVKGRFTITRDNSKN |
| | | | SAIPERFSGSNSGNT | | | TLNLQMNSLRAEDTAVYYC |
| | | | ATLTISRVEAGDEA | | | ARAPQWQLVAEAFDIWGQG |
| | | | DYYCQVWDTSSDG | | | TMVTVSS |
| | | | VVFGGGTKLTVL | | | |
| 5 | 11 | C5- | SYVLTQPPSVSVAP | 12 | C5- | QMQLVESGGGVVQPGRSLR |
| | | VL | GQTARITCGGNLIG | | VH | LSCAASGFTFRTYGMHWVR |
| | | | SKLVHWYQQKPGQ | | | QAPGKGLEWVAVIWYDGSN |
| | | | APVLVVYDDSDRP | | | KHYADSVKGRFTITRDNSKN |
| | | | SLIPERFSGSNSGNT | | | TLNLQMNSLRAEDTAVYYC |
| | | | ATLTISRVEAGDEA | | | ARAPQWELVAEAFDLWGQG |
| | | | DYYCQVWDTSSDG | | | TMVTVSS |
| | | | VVFGGGTKLTVL | | | |
| 6 | 13 | C8- | SYVLTQPPSVSVAP | 14 | C8- | QMQLVESGGGVVQPGRSLR |
| | | VL | GQTARITCGGNLLG | | VH | LSCAASGFTFRTYGMHWVR |
| | | | SKLVHWYQQKPGQ | | | QAPGKGLEWVAVIWYDGSN |
| | | | APVLVVYDDSDRP | | | KHYADSVKGRFTITRDNSKN |
| | | | SAIPERFSGSNSGNT | | | TLNLQMNSLRAEDTAVYYC |
| | | | ATLTISRVEAGDEA | | | ARAPQWELVAEAFDIWGQG |
| | | | DYYCQVWDSSSDH | | | TMVTVSS |
| | | | VVFGGGTKLTVL | | | |
| 7 | 15 | C6- | SYVLTQPPSVSVAP | 16 | C6- | QMQLVESGGGVVQPGRSLR |
| | | VL | GQTARITCGGNLIG | | VH | LSCAASGFTFRTYGMHWVR |
| | | | SKLVHWYQQKPGQ | | | QAPGKGLEWVAVIWYDGSN |
| | | | APVLVVYDDSDRP | | | KHYADSVKGRFTITRDNSKN |
| | | | SRIPERFSGSNSGNT | | | TLNLQMNSLRAEDTAVYYC |
| | | | ATLTISRVEAGDEA | | | ARAPQWMLVAEAFDLWGQ |
| | | | DYYCQVWDSSSDH | | | GTMVTVSS |
| | | | VVFGGGTKLTVL | | | |
| 8 | 17 | C3- | SYVLTQPPSVSVAP | 18 | C3- | QMQLVESGGGVVQPGRSLR |
| | | VL | GQTARITCGGNLIG | | VH | LSCAASGFTFRTYGMHWVR |
| | | | SKLVHWYQQKPGQ | | | QAPGKGLEWVAVIWYDGSN |
| | | | APVLVVYDDSDRP | | | KHYADSVKGRFTITRDNSKN |
| | | | SRIPERFSGSNSGNT | | | TLNLQMNSLRAEDTAVYYC |
| | | | ATLTISRVEAGDEA | | | ARAPQWEWVAEAFDLWGQ |
| | | | DYYCQVWDSSSDH | | | GTMVTVSS |
| | | | VVFGGGTKLTVL | | | |
| 9 | 19 | C37- | SYVLTQPPSVSVAP | 20 | C37- | QMQLVESGGGVVQPGRSLR |
| | | VL | GQTARITCGGNLIG | | VH | LSCAASGFTFRTYGMHWVR |
| | | | SKLVHWYQQKPGQ | | | QAPGKGLEWVAVIWYDGSN |
| | | | APVLVVYDDSDRP | | | KHYADSVKGRFTITRDNSKN |
| | | | SRIPERFSGSNSGNT | | | TLNLQMNSLRAEDTAVYYC |
| | | | ATLTISRVEAGDEA | | | ARAPQWELVAEAFDMWGQ |
| | | | DYYCQVWDSSSDH | | | GTMVTVSS |
| | | | VVFGGGTKLTVL | | | |
| 10 | 21 | C32- | SYVLTQPPSVSVAP | 22 | C32- | QMQLVESGGGVVQPGRSLR |
| | | VL | GQTARITCGGNLIG | | VH | LSCAASGFTFRTYGMHWVR |
| | | | SKLVHWYQQKPGQ | | | QAPGKGLEWVAVIWYDGSN |
| | | | APVLVVYDDSDRP | | | KHYADSVKGRFTITRDNSKN |
| | | | SKIPERFSGSNSGNT | | | TLNLQMNSLRAEDTAVYYC |
| | | | ATLTISRVEAGDEA | | | ARAPQWELVAEAFDLWGQG |
| | | | DYYCQVWDSSSDH | | | TMVTVSS |
| | | | VVFGGGTKLTVL | | | |
| 11 | 23 | C38- | SYVLTQPPSVSVAP | 24 | C38- | QMQLVESGGGVVQPGRSLR |
| | | VL | GQTARITCGGNLIG | | VH | LSCAASGFTFRTYGMHWVR |
| | | | SKLVHWYQQKPGQ | | | QAPGKGLEWVAVIWYDGSN |
| | | | APVLVVYDDSDRP | | | KHYADSVKGRFTITRDNSKN |
| | | | SDIPERFSGSNSGNT | | | TLNLQMNSLRAEDTAVYYC |
| | | | ATLTISRVEAGDEA | | | ARAPQWEYVAEAFDLWGQ |
| | | | DYYCQVWDTSSDH | | | GTMVTVSS |
| | | | VVFGGGTKLTVL | | | |
| 12 | 25 | C26- | SYVLTQPPSVSVAP | 26 | C26- | QMQLVESGGGVVQPGRSLR |
| | | VL | GQTARITCGGNIIGS | | VH | LSCAASGFTFRTYGMHWVR |
| | | | KLVHWYQQKPGQ | | | QAPGKGLEWVAVIWYDGSN |
| | | | APVLVVYDDGDRP | | | KHYADSVKGRFTITRDNSKN |
| | | | SGIPERFSGSNSGNT | | | TLNLQMNSLRAEDTAVYYC |
| | | | ATLTISRVEAGDEA | | | ARAPQWELTAEAFDLWGQG |
| | | | DYYCQVWDTSSDH | | | TMVTVSS |
| | | | VVFGGGTKLTVL | | | |
| 13 | 27 | C13- | SYVLTQPPSVSVAP | 28 | C13- | QMQLVESGGGVVQPGRSLR |
| | | VL | GQTARITCGGNLLG | | VH | LSCAASGFTFRTYGMHWVR |
| | | | SKLVHWYQQKPGQ | | | QAPGKGLEWVAVIWYDGSN |
| | | | APVLVVYDDGDRP | | | KHYADSVKGRFTITRDNSKN |
| | | | SLIPERFSGSNSGNT | | | TLNLQMNSLRAEDTAVYYC |
| | | | ATLTISRVEAGDEA | | | ARAPQWELVAEAFDLWGQG |
| | | | DYYCQVWDTSSDH | | | TMVTVSS |
| | | | VVFGGGTKLTVL | | | |
| 14 | 29 | C23- | SYVLTQPPSVSVAP | 30 | C23- | QMQLVESGGGVVQPGRSLR |
| | | VL | GQTARITCGGNLLG | | VH | LSCAASGFTFRTYGMHWVR |
| | | | SKLVHWYQQKPGQ | | | QAPGKGLEWVAVIWYDGSN |
| | | | APVLVVYDDSDRP | | | KHYADSVKGRFTITRDNSKN |
| | | | SEIPERFSGSNSGNT | | | TLNLQMNSLRAEDTAVYYC |
| | | | ATLTISRVEAGDEA | | | ARAPQWELTAEAFDIWGQG |
| | | | DYYCQVWDTSSDG | | | TMVTVSS |
| | | | VVFGGGTKLTVL | | | |
| 15 | 31 | C39- | SYVLTQPPSVSVAP | 32 | C39- | QMQLVESGGGVVQPGRSLR |
| | | VL | GQTARITCGGNLLG | | VH | LSCAASGFTFRTYGMHWVR |
| | | | SKLVHWYQQKPGQ | | | QAPGKGLEWVAVIWYDGSN |
| | | | APVLVVYDDSDRP | | | KHYADSVKGRFTITRDNSKN |
| | | | SWIPERFSGSNSGN | | | TLNLQMNSLRAEDTAVYYC |
| | | | TATLTISRVEAGDE | | | ARAPQWELTAEAFDLWGQG |
| | | | ADYYCQVWDTSSD | | | TMVTVSS |
| | | | HVVFGGGTKLTVL | | | |
| 16 | 33 | C17- | SYVLTQPPSVSVAP | 34 | C17- | QMQLVESGGGVVQPGRSLR |
| | | VL | GQTARITCGGNLIG | | VH | LSCAASGFTFRTYGMHWVR |
| | | | SKLVHWYQQKPGQ | | | QAPGKGLEWVAVIWYDGSN |
| | | | APVLVVYDDSDRP | | | KHYADSVKGRFTITRDNSKN |
| | | | SAIPERFSGSNSGNT | | | TLNLQMNSLRAEDTAVYYC |
| | | | ATLTISRVEAGDEA | | | ARAPQWELTSEAFDLWGQG |
| | | | DYYCQVWDTSSDH | | | TMVTVSS |
| | | | VVFGGGTKLTVL | | | |
| 17 | 35 | C45- | SYVLTQPPSVSVAP | 36 | C45- | QMQLVESGGGVVQPGRSLR |
| | | VL | GQTARITCGGNLIG | | VH | LSCAASGFTFRTYGMHWVR |
| | | | SKLVHWYQQKPGQ | | | QAPGKGLEWVAVIWYDGSN |
| | | | APVLVVYDDSDRP | | | KHYADSVKGRFTITRDNSKN |
| | | | SEIPERFSGSNSGNT | | | TLNLQMNSLRAEDTAVYYC |
| | | | ATLTISRVEAGDEA | | | ARAPQWELTSEAFDLWGQG |
| | | | DYYCQVWDSSSDG | | | TMVTVSS |
| | | | VVFGGGTKLTVL | | | |
| 18 | 37 | C7- | SYVLTQPPSVSVAP | 38 | C7- | QMQLVESGGGVVQPGRSLR |
| | | VL | GQTARITCGGNIIGS | | VH | LSCAASGFTFRTYGMHWVR |
| | | | KLVHWYQQKPGQ | | | QAPGKGLEWVAVIWYDGSN |
| | | | APVLVVYDDSDRP | | | KHYADSVKGRFTITRDNSKN |
| | | | SGIPERFSGSNSGNT | | | TLNLQMNSLRAEDTAVYYC |
| | | | ATLTISRVEAGDEA | | | ARAPQWLLVAEAFDLWGQG |
| | | | DYYCQVWDSGSD | | | TMVTVSS |
| | | | HVVFGGGTKLTVL | | | |
| 19 | 39 | C31- | SYVLTQPPSVSVAP | 40 | C31- | QMQLVESGGGVVQPGRSLR |
| | | VL | GQTARITCGGNIIGS | | VH | LSCAASGFTFRTYGMHWVR |
| | | | KLVHWYQQKPGQ | | | QAPGKGLEWVAVIWYDGSN |
| | | | APVLVVYDDSDRP | | | KHYADSVKGRFTITRDNSKN |
| | | | SDIPERFSGSNSGNT | | | TLNLQMNSLRAEDTAVYYC |
| | | | ATLTISRVEAGDEA | | | ARAPQWELVAEAFDLWGQG |
| | | | DYYCQVWDSGSD | | | TMVTVSS |
| | | | GVVFGGGTKLTVL | | | |
| 20 | 41 | C15- | SYVLTQPPSVSVAP | 42 | C15- | QMQLVESGGGVVQPGRSLR |
| | | VL | GQTARITCGGNLIG | | VH | LSCAASGFTFRTYGMHWVR |
| | | | SKLVHWYQQKPGQ | | | QAPGKGLEWVAVIWYDGSN |
| | | | APVLVVYDDGDRP | | | KHYADSVKGRFTITRDNSKN |
| | | | SWIPERFSGSNSGN | | | TLNLQMNSLRAEDTAVYYC |
| | | | TATLTISRVEAGDE | | | ARAPQWELVAEAFDLWGQG |
| | | | ADYYCQVWDSGS | | | TMVTVSS |
| | | | DGVVFGGGTKLTVL | | | |
| 21 | 43 | C35- | SYVLTQPPSVSVAP | 44 | C35- | QMQLVESGGGVVQPGRSLR |
| | | VL | GQTARITCGGNLIG | | VH | LSCAASGFTFRTYGMHWVR |
| | | | SKLVHWYQQKPGQ | | | QAPGKGLEWVAVIWYDGSN |
| | | | APVLVVYDDSDRP | | | KHYADSVKGRFTITRDNSKN |
| | | | SRIPERFSGSNSGNT | | | TLNLQMNSLRAEDTAVYYC |
| | | | ATLTISRVEAGDEA | | | ARAPQWVLVSEAFDLWGQG |
| | | | DYYCQVWDSGSD | | | TMVTVSS |
| | | | GVVFGGGTKLTVL | | | |
| 22 | 45 | C12 - | SYVLTQPPSVSVAP | 46 | C12- | QMQLVESGGGVVQPGRSLR |
| | | VL | GQTARITCGGNIIGS | | VH | LSCAASGFTFRTYGMHWVR |
| | | | KLVHWYQQKPGQ | | | QAPGKGLEWVAVIWYDGSN |
| | | | APVLVVYDDSDRP | | | KHYADSVKGRFTITRDNSKN |
| | | | SAIPERFSGSNSGNT | | | TLNLQMNSLRAEDTAVYYC |
| | | | ATLTISRVEAGDEA | | | ARAPQWELVAEAFDLWGQG |
| | | | DYYCQVWDSSSDG | | | TMVTVSS |
| | | | VVFGGGTKLTVL | | | |
| 23 | 47 | C4- | SYVLTQPPSVSVAP | 48 | C4- | QMQLVESGGGVVQPGRSLR |
| | | VL | GQTARITCGGNNIG | | VH | LSCAASGFTFRTYGMHWVR |
| | | | SKLVHWYQQKPGQ | | | QAPGKGLEWVAVISYDGSN |
| | | | APVLVVYDDSDRP | | | KHYADSVKGRFTITRDNSKN |
| | | | SGIPERFSGSNSGNT | | | TLNLQMNSLRAEDTAVYYC |
| | | | ATLTISRVEAGDEA | | | ARSPQWEWVHEAFDMWGQ |
| | | | DYYCQVWDSSSDG | | | GTMVTVSS |
| | | | VVFGGGTKLTVL | | | |
| 24 | 49 | C41- | SYVLTQPPSVSVAP | 50 | C41- | QMQLVESGGGVVQPGRSLR |
| | | VL | GQTARITCGGNILG | | VH | LSCAASGFTFRTYGMHWVR |
| | | | SKLVHWYQQKPGQ | | | QAPGKGLEWVAVISYDGSN |
| | | | APVLVVYDDSDRP | | | KHYADSVKGRFTITRDNSKN |
| | | | SEIPERFSGSNSGNT | | | TLNLQMNSLRAEDTAVYYC |
| | | | ATLTISRVEAGDEA | | | ARSPQWEWVHEAFDLWGQ |
| | | | DYYCQVWDTSSDG | | | GTMVTVSS |
| | | | VVFGGGTKLTVL | | | |
| 25 | 51 | C40- | SYVLTQPPSVSVAP | 52 | C40- | QMQLVESGGGVVQPGRSLR |
| | | VL | GQTARITCGGNNIG | | VH | LSCAASGFTFRTYGMHWVR |
| | | | SKLVHWYQQKPGQ | | | QAPGKGLEWVAVIWYDGSN |
| | | | APVLVVYDDSDRP | | | KHYADSVKGRFTITRDNSKN |
| | | | SRIPERFSGSNSGNT | | | TLNLQMNSLRAEDTAVYYC |
| | | | ATLTISRVEAGDEA | | | ARSPQWEWVHEAFDLWGQ |
| | | | DYYCQVWDTSSDG | | | GTMVTVSS |
| | | | VVFGGGTKLTVL | | | |
| 26 | 53 | C9- | SYVLTQPPSVSVAP | 54 | C9- | QMQLVESGGGVVQPGRSLR |
| | | VL | GQTARITCGGNLIG | | VH | LSCAASGFTFRTYGMHWVR |
| | | | SKLVHWYQQKPGQ | | | QAPGKGLEWVAVIWYDGSN |
| | | | APVLVVYDDGDRP | | | KHYADSVKGRFTITRDNSKN |
| | | | SWIPERFSGSNSGN | | | TLNLQMNSLRAEDTAVYYC |
| | | | TATLTISRVEAGDE | | | ARSPQWEWVHEAFDLWGQ |
| | | | ADYYCQVWDSSSD | | | GTMVTVSS |
| | | | GVVFGGGTKLTVL | | | |
| | 156 | 33.023 | SYVLTQPPSVSVAP | 155 | 33.023 | QMQLVESGGGVVQPGRSLR |
| | | VL | GQTARITCGGNLIG | | VH | LSCAASGFAFRTYGMHWVR |
| | | | SKLVHWYQQKPGQ | | | QAPGKGLEWVAVIWYDGSN |
| | | | APVLVVYDDSDRP | | | THYADSVKGRFTITRDNSKN |
| | | | SRIPERFSGSNSGNT | | | TLNLQMNSLRAEDTAVYYC |
| | | | ATLTISRVEAGDEA | | | ARAPQWYLSAEAFDLWGQG |
| | | | DYYCQVWDHSSD | | | TMVTVSS |
| | | | HVVFGGGTKLTVL | | | |
| | 160 | 33.025 | SYVLTQPPSVSVAP | 159 | 33.025 | QMQLVESGGGVVQPGRSLR |
| | | VL | GQTARITCGGNLIG | | VH | LSCAASGFTFRTYGMHWVR |
| | | | SKLVHWYQQKPGQ | | | QAPGKGLEWVADIWYDGSN |
| | | | APVLVVYDDSDRP | | | KHYADSVKGRFTITRDNSKN |
| | | | SLIPERFSGSNSGNT | | | TLNLQMNSLRAEDTAVYYC |
| | | | ATLTISRVEAGDEA | | | ARAPQWYLVAEPFDLWGQG |
| | | | DYYCQVWDSSSDG | | | TMVTVSS |
| | | | VVFGGGTKLTVL | | | |
| | 170 | 38.014 | SYVLTQPPSVSVAP | 169 | 38.014 | QMQLVESGGGVVQPGRSLR |
| | | VL | GQTARITCGGNLIG | | VH | LSCAASGFAFRTYGMHWVR |
| | | | AKLVHWYQQKPG | | | QAPGKGLEWVAVIWYDGSN |
| | | | QAPVLVVYDDSDR | | | THYADSVKGRFTITRDNSKN |
| | | | PSRIPERFSGSNSGN | | | TLNLQMNSLRAEDTAVYYC |
| | | | TATLTISRVEAGDE | | | VRAPQWYLSAEAFDLWGQG |
| | | | ADYYCQVWDHSS | | | TMVTVSS |
| | | | DHVVFGGGTKLTVL | | | |
| | 172 | 38.018 | SYVLTQPPSVSVAP | 171 | 38.018 | QMQLVESGGGVVQPGRSLR |
| | | VL | GQTARITCGGNLIG | | VH | LSCAASGFAFRTYGMHWVR |
| | | | SKLVHWYQQKPGQ | | | QAPGKGLEWVAVIWDDGSN |
| | | | APVLVVYDDSDRP | | | THYADSVKGRFTITRDNSKN |
| | | | SRIPERFSGSNSGNT | | | TLNLQMNSLRAEDTAVYYC |
| | | | ATLTISRVEAGDEA | | | ARAPQWYLSAEAFDLWGQG |
| | | | DYYCQVWDHSSD | | | TMVTVSS |
| | | | HVVFGGGTKLTVL | | | |
| | 174 | 38.019 | SYVLTQPPSVSVAP | 173 | 38.019 | QMQLVESGGGVVQPGRSLR |
| | | VL | GQTARITCGGNLIG | | VH | LSCAASGFAFRTYGMHWVR |
| | | | SKLVHWYQQKPGQ | | | QAPGKGLEWVAVIWYDGSN |
| | | | APVLVVYDDSDRP | | | THYADSVKGRFTITRDNSKN |
| | | | SRIPERFSGSNSGNT | | | TLNLQMNSLRAEDTAVYYC |
| | | | ATLTISRVEAGDEA | | | VRAPQWYLSAEAFDLWGQG |
| | | | DYYCQVWDYSSN | | | TMVTVSS |
| | | | HVVFGGGTKLTVL | | | |
| | 176 | 38.021 | SYVLTQPPSVSVAP | 175 | 38.021 | QMQLVESGGGVVQPGRSLR |
| | | VL | GQTARITCGGNLIG | | VH | LSCAASGFAFDTYGMHWVR |
| | | | SKLVHWYQQKPGQ | | | QAPGKGLEWVAVIWYDGSN |
| | | | APVLVVYDDSDRP | | | TVYADSVKGRFTITRDNSKN |
| | | | SRIPERFSGSNSGNT | | | TLNLQMNSLRAEDTAVYYC |
| | | | ATLTISRVEAGDEA | | | ARAPQWYLSAEAFDLWGQG |
| | | | DYYCQVWDHSSD | | | TMVTVSS |
| | | | HYVFGGGTKLTVL | | | |
| | 178 | 38.025 | SYVLTQPPSVSVAP | 177 | 38.025 | QMQLVESGGGVVQPGRSLR |
| | | VL | GETATITCGGNLIG | | VH | LSCAASGFAFRTYGMHWVR |
| | | | SKLVHWYQQKPGQ | | | QAPGKGLEWVAVIWYDGSN |
| | | | APVLVVYDDSDRP | | | THYADSVKGRFTITRDNSKN |
| | | | SRIPERFSGSNSGNT | | | TLNLQMNSLRAEDTAVYYC |
| | | | ATLTISRVEAGDEA | | | VRAPQWYLSAEAFDLWGQG |
| | | | DYYCQVWDHSSD | | | TMVTVSS |
| | | | HVVFGGGTKLTVL | | | |
| | 180 | 38.026 | SYVLTQPPSVSVAP | 179 | 38.026 | QMQLVESGGGVVQPGRSLR |
| | | VL | GQTATITCGGNLIG | | VH | LSCAASGFAFRTYGMHWVR |
| | | | SKLVHWYQQKPGQ | | | QAPGKGLEWVAVIWYDGSA |
| | | | APVLVVYDDSDRP | | | THYADSVKGRFTITRDNSKN |
| | | | SRIPERFSGSNIGNT | | | TLNLQMNSLRAEDTAVYYC |
| | | | ATLTISRVEAGDEA | | | VRAPQWYLSAEAFDLWGQG |
| | | | DYYCQVWDHSSD | | | TMVTVSS |
| | | | HVVFGGGTKLTVL | | | |
| | 182 | 38.040 | SYVLTQPPSVSVAP | 181 | 38.040 | QMQLVESGGGVVQPGRSLR |
| | | VL | GQTARITCGGNLIG | | VH | LSCAASGFDFRTYGMHWVR |
| | | | TKLVHWYQQKPG | | | QAPGKGLEWVAVIWYDGSI |
| | | | QAPVLVVYDDSDR | | | THYADSVKGRFTITRDNSKN |
| | | | PSRIPERFSGSNSGN | | | TLNLQMNSLRAEDTAVYYC |
| | | | TATLTISRVEAGDE | | | VRAPQWYLTAEAFDLWGQG |
| | | | ADYYCQVWDHNE | | | TMVTVSS |
| | | | DEVVFGGGTKLTV | | | |
| | | | L | | | |

TABLE 2
Nucleotide Sequences Encoding Engineered
dual Binding scFv: Variable Heavy
chain (VH)-Linker-Variable Light chain (VL)

| | SEQ | | |
|---|---|---|---|
| Ab | ID | | |
| # | NO: | Name | VH-Linker-VL Sequence |
| | 109 | Template | CAAATGCAGCTGGTCGAGTCTGGCGGTGGGGTAGTGCAAC |
| | | VH-L-VL | CAGGCCGTTCTCTGCGTCTTAGCTGCGCCGCATCTGGTTTT |
| | | | ACCTTTCGTACCTACGGTATGCACTGGGTGCGTCAGGCACC |
| | | | AGGCAAAGGTCTGGAATGGGTCGCAGTAATCTGGTATGAT |
| | | | GGTAGCAATAAACACTATGCTGACTCAGTCAAAGGCCGTTT |
| | | | CACCATCACCCGTGATAACAGCAAGAACACTCTTAACTTAC |
| | | | AGATGAACTCTCTGCGTGCCGAAGACACCGCAGTTTATTAC |
| | | | TGTGCCCGTGCACCACAGTGGGAATTAGTACACGAAGCAT |
| | | | TCGATATCTGGGGTCAGGGTACTATGGTGACCGTTAGCTCT |
| | | | GGCGGTGGTGGTAGCGGAGGCGGAGGATCAGGTGGAGGC |
| | | | GGCAGTTCTTACGTGCTGACTCAACCACCATCAGTGTCTGT |
| | | | AGCACCAGGCCAGACCGCACGTATTACCTGTGGCGGTAAC |
| | | | AATCTGGGCTCTAAGTCTGTTCACTGGTATCAGCAAAAACC |
| | | | AGGCCAGGCACCAGTACTGGTTGTGTACGATGATTCCGATC |
| | | | GTCCAAGCTGGATTCCAGAGCGTTTCAGCGGCTCTAATTCC |
| | | | GGCAACACCGCTACTCTGACTATTTCCCGTGGGGAAGCCGG |
| | | | CGATGAAGCCGACTACTATTGCCAGGTCTGGGACTCTTCTT |
| | | | CCGACCATGTAGTCTTTGGCGGGGGCACCAAACTGACCGTT |
| | | | TTG |
| 1 | 110 | C2- | CAAATGCAGCTGGTCGAGTCTGGCGGTGGGGTAGTGCAAC |
| | | VH-L-VL | CAGGCCGTTCTCTGCGTCTTAGCTGCGCCGCATCTGGTTTT |
| | | | ACCTTTCGTACCTACGGTATGCACTGGGTGCGTCAGGCACC |
| | | | AGGCAAAGGTCTGGAATGGGTCGCAGTAATCTGGTATGAT |
| | | | GGTAGCAATAAACACTATGCTGACTCAGTCAAAGGCCGTTT |
| | | | CACCATCACCCGTGATAACAGCAAGAACACTCTTAACTTAC |
| | | | AGATGAACTCTCTGCGTGCCGAAGACACCGCCGTTTACTAC |
| | | | TGTGCCCGTGCACCACAGTGGGAATTAACGGCGGAAGCAT |
| | | | TCGATATTTGGGGCCAGGGCACTATGGTGACCGTTAGCTCT |
| | | | GGCGGTGGTGGTAGCGGAGGCGGAGGATCAGGTGGAGGC |
| | | | GGCAGTTCTTACGTGCTGACTCAACCACCATCAGTGTCTGT |
| | | | AGCACCAGGCCAGACCGCACGTATTACCTGTGGCGGTAAC |
| | | | CTGATCGGCTCTAAGCTGGTTCACTGGTATCAGCAAAAACC |
| | | | AGGCCAGGCACCAGTACTGGTTGTGTACGATGATAGCGAT |
| | | | CGTCCAAGCCTGATTCCAGAGCGTTTCAGCGGCTCTAATTC |
| | | | CGGCAACACCGCTACTCTGACTATTTCCCGTGTTGAAGCCG |
| | | | GCGATGAAGCCGACTACTATTGCCAGGTCTGGGACTCTAGC |
| | | | TCCGACGGTGTAGTCTTTGGCGGGGGCACCAAACTGACCGT |
| | | | TTTG |
| 2 | 111 | C27- | CAAATGCAGCTGGTCGAGTCTGGCGGTGGGGTAGTGCAAC |
| | | VH-L-VL | CAGGCCGTTCTCTGCGTCTTAGCTGCGCCGCATCTGGTTTT |
| | | | ACCTTTCGTACCTACGGTATGCACTGGGTGCGTCAGGCACC |
| | | | AGGCAAAGGTCTGGAATGGGTCGCAGTAATCTGGTATGAT |
| | | | GGTAGCAATAAACACTATGCTGACTCAGTCAAAGGCCGTTT |
| | | | CACCATCACCCGTGATAACAGCAAGAACACTCTTAACTTAC |
| | | | AGATGAACTCTCTGCGTGCCGAAGACACCGCCGTTTACTAC |
| | | | TGTGCCCGTGCACCACAGTGGGAATTAGTAGCGGAAGCAT |
| | | | TCGATCTGTGGGGCCAGGGCACTATGGTGACCGTTAGCTCT |
| | | | GGCGGTGGTGGTAGCGGAGGCGGAGGATCAGGTGGAGGC |
| | | | GGCAGTTCTTACGTGCTGACTCAACCACCATCAGTGTCTGT |
| | | | AGCACCAGGCCAGACCGCACGTATTACCTGTGGCGGTAAC |
| | | | CTGATCGGCTCTAAGCTGGTTCACTGGTATCAGCAAAAACC |
| | | | AGGCCAGGCACCAGTACTGGTTGTGTACGATGATAGCGAT |
| | | | CGTCCAAGCCTGATTCCAGAGCGTTTCAGCGGCTCTAATTC |
| | | | CGGCAACACCGCTACTCTGACTATTTCCCGTGTTGAAGCCG |
| | | | GCGATGAAGCCGACTACTATTGCCAGGTCTGGGACTCTAGC |
| | | | TCCGACGGTGTAGTCTTTGGCGGGGGCACCAAACTGACCGT |
| | | | TTTG |
| 3 | 112 | C19- | CAAATGCAGCTGGTCGAGTCTGGCGGTGGGGTAGTGCAAC |
| | | VH-L-VL | CAGGCCGTTCTCTGCGTCTTAGCTGCGCCGCATCTGGTTTT |
| | | | ACCTTTCGTACCTACGGTATGCACTGGGTGCGTCAGGCACC |
| | | | AGGCAAAGGTCTGGAATGGGTCGCAGTAATCTGGTATGAT |
| | | | GGTAGCAATAAACACTATGCTGACTCAGTCAAAGGCCGTTT |
| | | | CACCATCACCCGTGATAACAGCAAGAACACTCTTAACTTAC |
| | | | AGATGAACTCTCTGCGTGCCGAAGACACCGCCGTTTACTAC |
| | | | TGTGCCCGTGCACCACAGTGGCAGTTAGTAGCGGAAGCAT |
| | | | TCGATATTTGGGGCCAGGGCACTATGGTGACCGTTAGCTCT |
| | | | GGCGGTGGTGGTAGCGGAGGCGGAGGATCAGGTGGAGGC |
| | | | GGCAGTTCTTACGTGCTGACTCAACCACCATCAGTGTCTGT |
| | | | AGCACCAGGCCAGACCGCACGTATTACCTGTGGCGGTAAC |
| | | | CTGATCGGCTCTAAGCTGGTTCACTGGTATCAGCAAAAACC |
| | | | AGGCCAGGCACCAGTACTGGTTGTGTACGATGATAGCGAT |
| | | | CGTCCAAGCCTTATTCCAGAGCGTTTCAGCGGCTCTAATTC |
| | | | CGGCAACACCGCTACTCTGACTATTTCCCGTGTTGAAGCCG |
| | | | GCGATGAAGCCGACTACTATTGCCAGGTCTGGGACACCAG |
| | | | CTCCGACGGTGTAGTCTTTGGCGGGGGCACCAAACTGACC |
| | | | GTTTTG |
| 4 | 113 | C43- | CAAATGCAGCTGGTCGAGTCTGGCGGTGGGGTAGTGCAAC |
| | | VH-L-VL | CAGGCCGTTCTCTGCGTCTTAGCTGCGCCGCATCTGGTTTT |
| | | | ACCTTTCGTACCTACGGTATGCACTGGGTGCGTCAGGCACC |
| | | | AGGCAAAGGTCTGGAATGGGTCGCAGTAATCTGGTATGAT |
| | | | GGTAGCAATAAACACTATGCTGACTCAGTCAAAGGCCGTTT |
| | | | CACCATCACCCGTGATAACAGCAAGAACACTCTTAACTTAC |
| | | | AGATGAACTCTCTGCGTGCCGAAGACACCGCCGTTTACTAC |
| | | | TGTGCCCGTGCACCACAGTGGCAGTTAGTAGCCGAAGCATT |
| | | | CGATATATGGGGCCAGGGCACTATGGTGACCGTTAGCTCTG |
| | | | GCGGTGGTGGTAGCGGAGGCGGAGGATCAGGTGGAGGCG |
| | | | GCAGTTCTTACGTGCTGACTCAACCACCATCAGTGTCTGTA |
| | | | GCACCAGGCCAGACCGCACGTATTACCTGTGGCGGTAACC |
| | | | TGATCGGCTCTAAGCTGGTTCACTGGTATCAGCAAAAACCA |
| | | | GGCCAGGCACCAGTACTGGTTGTGTACGATGATAGCGATC |
| | | | GTCCAAGCGCTATTCCAGAGCGTTTCAGCGGCTCTAATTCC |
| | | | GGCAACACCGCTACTCTGACTATTTCCCGTGTTGAAGCCGG |
| | | | CGATGAAGCCGACTACTATTGCCAGGTCTGGGACACTAGCT |
| | | | CCGACGGTGTAGTCTTTGGCGGGGGCACCAAACTGACCGTT |
| | | | TTG |
| 5 | 114 | C5- | CAAATGCAGCTGGTCGAGTCTGGCGGTGGGGTAGTGCAAC |
| | | VH-L-VL | CAGGCCGTTCTCTGCGTCTTAGCTGCGCCGCATCTGGTTTT |
| | | | ACCTTTCGTACCTACGGTATGCACTGGGTGCGTCAGGCACC |
| | | | AGGCAAAGGTCTGGAATGGGTCGCAGTAATCTGGTATGAT |
| | | | GGTAGCAATAAACACTATGCTGACTCAGTCAAAGGCCGTTT |
| | | | CACCATCACCCGTGATAACAGCAAGAACACTCTTAACTTAC |
| | | | AGATGAACTCTCTGCGTGCCGAAGACACCGCCGTTTACTAC |
| | | | TGTGCCCGTGCACCACAGTGGGAATTAGTGGCGGAAGCAT |
| | | | TCGATCTTTGGGGCCAGGGCACTATGGTGACCGTTAGCTCT |
| | | | GGTGGTGGTGGTAGCGGAGGCGGAGGATCAGGTGGAGGCG |
| | | | GCAGTTCTTACGTGCTGACTCAACCACCATCAGTGTCTGTA |
| | | | GCACCAGGCCAGACCGCACGTATTACCTGTGGCGGTAACC |
| | | | TGATCGGCTCTAAGCTGGTTCACTGGTATCAGCAAAAACCA |
| | | | GGCCAGGCACCAGTACTGGTTGTGTACGATGATAGCGATC |
| | | | GTCCAAGCCTTATTCCAGAGCGTTTCAGCGGCTCTAATTCC |
| | | | GGCAACACCGCTACTCTGACTATTTCCCGTGTTGAAGCCGG |
| | | | CGATGAAGCCGACTACTATTGCCAGGTCTGGGACACCAGC |
| | | | TCCGACGGTGTAGTCTTTGGCGGGGGCACCAAACTGACCGT |
| | | | TTTG |
| 6 | 115 | C8- | CAAATGCAGCTGGTCGAGTCTGGCGGTGGGGTAGTGCAAC |
| | | VH-L-VL | CAGGCCGTTCTCTGCGTCTTAGCTGCGCCGCATCTGGTTTT |
| | | | ACCTTTCGTACCTACGGTATGCACTGGGTGCGTCAGGCACC |
| | | | AGGCAAAGGTCTGGAATGGGTCGCAGTAATCTGGTATGAT |
| | | | GGTAGCAATAAACACTATGCTGACTCAGTCAAAGGCCGTTT |
| | | | CACCATCACCCGTGATAACAGCAAGAACACTCTTAACTTAC |
| | | | AGATGAACTCTCTGCGTGCCGAAGACACCGCCGTTTACTAC |
| | | | TGTGCCCGTGCACCACAGTGGGAATTGGTAGCCGAAGCAT |
| | | | TCGATATCTGGGGCCAGGGCACTATGGTGACCGTTAGCTCT |
| | | | GGCGGTGGTGGTAGCGGAGGCGGAGGATCAGGTGGAGGC |
| | | | GGCAGTTCTTACGTGCTGACTCAACCACCATCAGTGTCTGT |
| | | | AGCACCAGGCCAGACCGCACGTATTACCTGTGGCGGTAAC |
| | | | CTGCTGGGCTCTAAGCTGGTTCACTGGTATCAGCAAAAACC |
| | | | AGGCCAGGCACCAGTACTGGTTGTGTACGATGATAGCGAT |
| | | | CGTCCAAGCGCAATTCCAGAGCGTTTCAGCGGCTCTAATTC |
| | | | CGGCAACACCGCTACTCTGACTATTTCCCGTGTTGAAGCCG |
| | | | GCGATGAAGCCGACTACTATTGCCAGGTCTGGGACTCTAGC |
| | | | TCCGACCACGTAGTCTTTGGCGGGGGCACCAAACTGACCGT |
| | | | TTTG |
| 7 | 116 | C6- | CAAATGCAGCTGGTCGAGTCTGGCGGTGGGGTAGTGCAAC |
| | | VH-L-VL | CAGGCCGTTCTCTGCGTCTTAGCTGCGCCGCATCTGGTTTT |
| | | | ACCTTTCGTACCTACGGTATGCACTGGGTGCGTCAGGCACC |
| | | | AGGCAAAGGTCTGGAATGGGTCGCAGTAATCTGGTATGAT |
| | | | GGTAGCAATAAACACTATGCTGACTCAGTCAAAGGCCGTTT |
| | | | CACCATCACCCGTGATAACAGCAAGAACACTCTTAACTTAC |
| | | | AGATGAACTCTCTGCGTGCCGAAGACACCGCCGTTTACTAC |
| | | | TGTGCCCGTGCACCACAGTGGATGTTAGTAGCGGAAGCATT |
| | | | CGATCTATGGGGCCAGGGCACTATGGTGACCGTTAGCTCTG |
| | | | GCGGTGGTGGTAGCGGAGGCGGAGGATCAGGTGGAGGCG |
| | | | GCAGTTCTTACGTGCTGACTCAACCACCATCAGTGTCTGTA |
| | | | GCACCAGGCCAGACCGCACGTATTACCTGTGGCGGTAACC |
| | | | TGATCGGCTCTAAGCTGGTTCACTGGTATCAGCAAAAACCA |
| | | | GGGCAGGCACCAGTACTGGTTGTGTACGATGATAGCGATC |
| | | | GTCCAAGCCGGATTCCAGAGCGTTTCAGCGGCTCTAATTCC |
| | | | GGCAACACCGCTACTCTGACTATTTCCCGTGTTGAAGCCGG |
| | | | CGATGAAGCCGACTACTATTGCCAGGTCTGGGACTCCAGCT |
| | | | CCGACCATGTAGTCTTTGGCGGGGGCACCAAACTGACCGTT |
| | | | TTG |
| 8 | 117 | C3- | CAAATGCAGCTGGTCGAGTCTGGCGGTGGGGTAGTGCAAC |
| | | VH-L-VL | CAGGCCGTTCTCTGCGTCTTAGCTGCGCCGCATCTGGTTTT |
| | | | ACCTTTCGTACCTACGGTATGCACTGGGTGCGTCAGGCACC |
| | | | AGGCAAAGGTCTGGAATGGGTCGCAGTAATCTGGTATGAT |
| | | | GGTAGCAATAAACACTATGCTGACTCAGTCAAAGGCCGTTT |
| | | | CACCATCACCCGTGATAACAGCAAGAACACTCTTAACTTAC |
| | | | AGATGAACTCTCTGCGTGCCGAAGACACCGCCGTTTACTAC |
| | | | TGTGCCCGTGCACCACAGTGGGAATGGGTAGCCGAAGCAT |
| | | | TCGATCTGTGGGGCCAGGGCACTATGGTGACCGTTAGCTCT |
| | | | GGCGGTGGTGGTAGCGGAGGCGGAGGATCAGGTGGAGGC |
| | | | GGCAGTTCTTACGTGCTGACTCAACCACCATCAGTGTCTGT |
| | | | AGCACCAGGCCAGACCGCACGTATTACCTGTGGCGGTAAC |
| | | | CTGATCGGCTCTAAGCTGGTTCACTGGTATCAGCAAAAACC |
| | | | AGGCCAGGCACCAGTACTGGTTGTGTACGATGATAGCGAT |
| | | | CGTCCAAGCCGGATTCCAGAGCGTTTCAGCGGCTCTAATTC |
| | | | CGGCAACACCGCTACTCTGACTATTTCCCGTGTTGAAGCCG |
| | | | GCGATGAAGCCGACTACTATTGCCAGGTCTGGGACTCCAG |
| | | | CTCCGACCACGTAGTCTTTGGCGGGGGCACCAAACTGACC |
| | | | GTTTTG |
| 9 | 118 | C37- | CAAATGCAGCTGGTCGAGTCTGGCGGTGGGGTAGTGCAAC |
| | | VH-L-VL | CAGGCCGTTCTCTGCGTCTTAGCTGCGCCGCATCTGGTTTT |
| | | | ACCTTTCGTACCTACGGTATGCACTGGGTGCGTCAGGCACC |
| | | | AGGCAAAGGTCTGGAATGGGTCGCAGTAATCTGGTATGAT |
| | | | GGTAGCAATAAACACTATGCTGACTCAGTCAAAGGCCGTTT |
| | | | CACCATCACCCGTGATAACAGCAAGAACACTCTTAACTTAC |
| | | | AGATGAACTCTCTGCGTGCCGAAGACACCGCCGTTTACTAC |
| | | | TGTGCCCGTGCACCACAGTGGGAATTAGTAGCCGAGGCAT |
| | | | TCGATATGTGGGGCCAGGGCACTATGGTGACCGTTAGCTCT |
| | | | GGCGGTGGTGGTAGCGGAGGCGGAGGATCAGGTGGAGGC |
| | | | GGCAGTTCTTACGTGCTGACTCAACCACCATCAGTGTCTGT |
| | | | AGCACCAGGCCAGACCGCACGTATTACCTGTGGCGGTAAC |
| | | | CTGATCGGCTCTAAGCTGGTTCACTGGTATCAGCAAAAACC |
| | | | AGGCCAGGCACCAGTACTGGTTGTGTACGATGATAGCGAT |
| | | | CGTCCAAGCCGGATTCCAGAGCGTTTCAGCGGCTCTAATTC |
| | | | CGGCAACACCGCTACTCTGACTATTTCCCGTGTTGAAGCCG |
| | | | GCGATGAAGCCGACTACTATTGCCAGGTCTGGGACTCTAGC |
| | | | TCCGACCATGTAGTCTTTGGCGGGGGCACCAAACTGACCGT |
| | | | TTTG |
| 10 | 119 | C32- | CAAATGCAGCTGGTCGAGTCTGGCGGTGGGGTAGTGCAAC |
| | | VH-L-VL | CAGGCCGTTCTCTGCGTCTTAGCTGCGCCGCATCTGGTTTT |
| | | | ACCTTTCGTACCTACGGTATGCACTGGGTGCGTCAGGCACC |
| | | | AGGCAAAGGTCTGGAATGGGTCGCAGTAATCTGGTATGAT |
| | | | GGTAGCAATAAACACTATGCTGACTCAGTCAAAGGCCGTTT |
| | | | CACCATCACCCGTGATAACAGCAAGAACACTCTTAACTTAC |
| | | | AGATGAACTCTCTGCGTGCCGAAGACACCGCCGTTTACTAC |
| | | | TGTGCCCGTGCACCACAGTGGGAATTAGTAGCCGAAGCAT |
| | | | TCGATCTGTGGGGCCAGGGCACTATGGTGACCGTTAGCTCT |
| | | | GGCGGTGGTGGTAGCGGAGGCGGAGGATCAGGTGGAGGC |
| | | | GGCAGTTCTTACGTGCTGACTCAACCACCATCAGTGTCTGT |
| | | | AGCACCAGGCCAGACCGCACGTATTACCTGTGGCGGTAAC |
| | | | CTGATCGGCTCTAAGCTGGTTCACTGGTATCAGCAAAAACC |
| | | | AGGCCAGGCACCAGTACTGGTTGTGTACGATGATAGCGAT |
| | | | CGTCCAAGCAAAATTCCAGAGCGTTTCAGCGGCTCTAATTC |
| | | | CGGCAACACCGCTACTCTGACTATTTCCCGTGTTGAAGCCG |
| | | | GCGATGAAGCCGACTACTATTGCCAGGTCTGGGACTCCAG |
| | | | CTCCGACCATGTAGTCTTTGGCGGGGGCACCAAACTGACCG |
| | | | TTTTG |
| 11 | 120 | C38- | CAAATGCAGCTGGTCGAGTCTGGCGGTGGGGTAGTGCAAC |
| | | VH-L-VL | CAGGCCGTTCTCTGCGTCTTAGCTGCGCCGCATCTGGTTTT |
| | | | ACCTTTCGTACCTACGGTATGCACTGGGTGCGTCAGGCACC |
| | | | AGGCAAAGGTCTGGAATGGGTCGCAGTAATCTGGTATGAT |
| | | | GGTAGCAATAAACACTATGCTGACTCAGTCAAAGGCCGTTT |
| | | | CACCATCACCCGTGATAACAGCAAGAACACTCTTAACTTAC |
| | | | AGATGAACTCTCTGCGTGCCGAAGACACCGCCGTTTACTAC |
| | | | TGTGCCCGTGCACCACAGTGGGAATACGTAGCGGAAGCAT |
| | | | TCGATCTGTGGGGCCAGGGCACTATGGTGACCGTTAGCTCT |
| | | | GGCGGTGGTGGTAGCGGAGGCGGAGGATCAGGTGGAGGC |
| | | | GGCAGTTCTTACGTGCTGACTCAACCACCATCAGTGTCTGT |
| | | | AGCACCAGGCCAGACCGCACGTATTACCTGTGGCGGTAAC |
| | | | CTGATCGGCTCTAAGCTGGTTCACTGGTATCAGCAAAAACC |
| | | | AGGCCAGGCACCAGTACTGGTTGTGTACGATGATAGCGAT |
| | | | CGTCCAAGCGATATTCCAGAGCGTTTCAGCGGCTCTAATTC |
| | | | CGGCAACACCGCTACTCTGACTATTTCCCGTGTTGAAGCCG |
| | | | GCGATGAAGCCGACTACTATTGCCAGGTCTGGGACACCAG |
| | | | CTCCGACCATGTAGTCTTTGGCGGGGGCACCAAACTGACCG |
| | | | TTTTG |
| 12 | 121 | C26- | CAAATGCAGCTGGTCGAGTCTGGCGGTGGGGTAGTGCAAC |
| | | VH-L-VL | CAGGCCGTTCTCTGCGTCTTAGCTGCGCCGCATCTGGTTTT |
| | | | ACCTTTCGTACCTACGGTATGCACTGGGTGCGTCAGGCACC |
| | | | AGGCAAAGGTCTGGAATGGGTCGCAGTAATCTGGTATGAT |
| | | | GGTAGCAATAAACACTATGCTGACTCAGTCAAAGGCCGTTT |
| | | | CACCATCACCCGTGATAACAGCAAGAACACTCTTAACTTAC |
| | | | AGATGAACTCTCTGCGTGCCGAAGACACCGCCGTTTACTAC |
| | | | TGTGCCCGTGCACCACAGTGGGAATTAACGGCCGAAGCAT |
| | | | TCGATCTTTGGGGCCAGGGCACTATGGTGACCGTTAGCTCT |
| | | | GGCGGTGGTGGTAGCGGAGGCGGAGGATCAGGTGGAGGC |
| | | | GGCAGTTCTTACGTGCTGACTCAACCACCATCAGTGTCTGT |
| | | | AGCACCAGGCCAGACCGCACGTATTACCTGTGGCGGTAAC |
| | | | ATCATCGGCTCTAAGCTGGTTCACTGGTATCAGCAAAAACC |
| | | | AGGCCAGGCACCAGTACTGGTTGTGTACGATGATGGCGAT |
| | | | CGTCCAAGCGGTATTCCAGAGCGTTTCAGCGGCTCTAATTC |
| | | | CGGCAACACCGCTACTCTGACTATTTCCCGTGTTGAAGCCG |
| | | | GCGATGAAGCCGACTACTATTGCCAGGTCTGGGACACTAG |
| | | | CTCCGACCACGTAGTCTTTGGCGGGGGCACCAAACTGACC |
| | | | GTTTTG |
| 13 | 122 | C13- | CAAATGCAGCTGGTCGAGTCTGGCGGTGGGGTAGTGCAAC |
| | | VH-L-VL | CAGGCCGTTCTCTGCGTCTTAGCTGCGCCGCATCTGGTTTT |
| | | | ACCTTTCGTACCTACGGTATGCACTGGGTGCGTCAGGCACC |
| | | | AGGCAAAGGTCTGGAATGGGTCGCAGTAATCTGGTATGAT |
| | | | GGTAGCAATAAACACTATGCTGACTCAGTCAAAGGCCGTTT |
| | | | CACCATCACCCGTGATAACAGCAAGAACACTCTTAACTTAC |
| | | | AGATGAACTCTCTGCGTGCCGAAGACACCGCCGTTTACTAC |
| | | | TGTGCCCGTGCACCACAGTGGGAATTAGTGGCGGAAGCAT |
| | | | TCGATCTTTGGGGCCAGGGCACTATGGTGACCGTTAGCTCT |
| | | | GGCGGTGGTGGTAGCGGAGGCGGAGGATCAGGTGGAGGC |
| | | | GGCAGTTCTTACGTGCTGACTCAACCACCATCAGTGTCTGT |
| | | | AGCACCAGGCCAGACCGCACGTATTACCTGTGGCGGTAAC |
| | | | CTGCTGGGCTCTAAGCTGGTTCACTGGTATCAGCAAAAACC |
| | | | AGGCCAGGCACCAGTACTGGTTGTGTACGATGATGGCGAT |
| | | | CGTCCAAGCCTGATTCCAGAGCGTTTCAGCGGCTCTAATTC |
| | | | CGGCAACACCGCTACTCTGACTATTTCCCGTGTTGAAGCCG |
| | | | GCGATGAAGCCGACTACTATTGCCAGGTCTGGGACACTAG |
| | | | CTCCGACCACGTAGTCTTTGGCGGGGGCACCAAACTGACC |
| | | | GTTTTG |
| 14 | 123 | C23- | CAAATGCAGCTGGTCGAGTCTGGCGGTGGGGTAGTGCAAC |
| | | VH-L-VL | CAGGCCGTTCTCTGCGTCTTAGCTGCGCCGCATCTGGTTTT |
| | | | ACCTTTCGTACCTACGGTATGCACTGGGTGCGTCAGGCACC |
| | | | AGGCAAAGGTCTGGAATGGGTCGCAGTAATCTGGTATGAT |
| | | | GGTAGCAATAAACACTATGCTGACTCAGTCAAAGGCCGTTT |
| | | | CACCATCACCCGTGATAACAGCAAGAACACTCTTAACTTAC |
| | | | AGATGAACTCTCTGCGTGCCGAAGACACCGCCGTTTACTAC |
| | | | TGTGCCCGTGCACCACAGTGGGAATTAACGGCGGAAGCAT |
| | | | TCGATATTTGGGGCCAGGGCACTATGGTGACCGTTAGCTCT |
| | | | GGCGGTGGTGGTAGCGGAGGCGGAGGATCAGGTGGAGGC |
| | | | GGCAGTTCTTACGTGCTGACTCAACCACCATCAGTGTCTGT |
| | | | AGCACCAGGCCAGACCGCACGTATTACCTGTGGCGGTAAC |
| | | | CTGCTGGGCTCTAAGCTGGTTCACTGGTATCAGCAAAAACC |
| | | | AGGCCAGGCACCAGTACTGGTTGTGTACGATGATAGCGAT |
| | | | CGTCCAAGCGAAATTCCAGAGCGTTTCAGCGGCTCTAATTC |
| | | | CGGCAACACCGCTACTCTGACTATTTCCCGTGTTGAAGCCG |
| | | | GCGATGAAGCCGACTACTATTGCCAGGTCTGGGACACCAG |
| | | | CTCCGACGGTGTAGTCTTTGGCGGGGGCACCAAACTGACC |
| | | | GTTTTG |
| 15 | 124 | C39- | CAAATGCAGCTGGTCGAGTCTGGCGGTGGGGTAGTGCAAC |
| | | VH-L-VL | CAGGCCGTTCTCTGCGTCTTAGCTGCGCCGCATCTGGTTTT |
| | | | ACCTTTCGTACCTACGGTATGCACTGGGTGCGTCAGGCACC |
| | | | AGGCAAAGGTCTGGAATGGGTCGCAGTAATCTGGTATGAT |
| | | | GGTAGCAATAAACACTATGCTGACTCAGTCAAAGGCCGTTT |
| | | | CACCATCACCCGTGATAACAGCAAGAACACTCTTAACTTAC |
| | | | AGATGAACTCTCTGCGTGCCGAAGACACCGCCGTTTACTAC |
| | | | TGTGCCCGTGCACCACAGTGGGAATTAACGGCCGAAGCAT |
| | | | TCGATCTTTGGGGCCAGGGCACTATGGTGACCGTTAGCTCT |
| | | | GGCGGTGGTGGTAGCGGAGGCGGAGGATCAGGTGGAGGC |
| | | | GGCAGTTCTTACGTGCTGACTCAACCACCATCAGTGTCTGT |
| | | | AGCACCAGGCCAGACCGCACGTATTACCTGTGGCGGTAAC |
| | | | CTGCTGGGCTCTAAGCTGGTTCACTGGTATCAGCAAAAACC |
| | | | AGGCCAGGCACCAGTACTGGTTGTGTACGATGATAGCGAT |
| | | | CGTCCAAGCTGGATTCCAGAGCGTTTCAGCGGCTCTAATTC |
| | | | CGGCAACACCGCTACTCTGACTATTTCCCGTGTTGAAGCCG |
| | | | GCGATGAAGCCGACTACTATTGCCAGGTCTGGGACACTAG |
| | | | CTCCGACCATGTAGTCTTTGGCGGGGGCACCAAACTGACCG |
| | | | TTTTG |
| 16 | 125 | C17- | CAAATGCAGCTGGTCGAGTCTGGCGGTGGGGTAGTGCAAC |
| | | VH-L-VL | CAGGCCGTTCTCTGCGTCTTAGCTGCGCCGCATCTGGTTTT |
| | | | ACCTTTCGTACCTACGGTATGCACTGGGTGCGTCAGGCACC |
| | | | AGGCAAAGGTCTGGAATGGGTCGCAGTAATCTGGTATGAT |
| | | | GGTAGCAATAAACACTATGCTGACTCAGTCAAAGGCCGTTT |
| | | | CACCATCACCCGTGATAACAGCAAGAACACTCTTAACTTAC |
| | | | AGATGAACTCTCTGCGTGCCGAAGACACCGCCGTTTACTAC |
| | | | TGTGCCCGTGCACCACAGTGGGAATTAACGTCGGAAGCAT |
| | | | TCGATCTTTGGGGCCAGGGCACTATGGTGACCGTTAGCTCT |
| | | | GGCGGTGGTGGTAGCGGAGGCGGAGGATCAGGTGGAGGC |
| | | | GGCAGTTCTTACGTGCTGACTCAACCACCATCAGTGTCTGT |
| | | | AGCACCAGGCCAGACCGCACGTATTACCTGTGGCGGTAAC |
| | | | CTGATCGGCTCTAAGCTGGTTCACTGGTATCAGCAAAAACC |
| | | | AGGCCAGGCACCAGTACTGGTTGTGTACGATGATAGCGAT |
| | | | CGTCCAAGCGCAATTCCAGAGCGTTTCAGCGGCTCTAATTC |
| | | | CGGCAACACCGCTACTCTGACTATTTCCCGTGTTGAAGCCG |
| | | | GCGATGAAGCCGACTACTATTGCCAGGTCTGGGACACCAG |
| | | | CTCCGACCACGTAGTCTTTGGCGGGGGCACCAAACTGACC |
| | | | GTTTTG |
| 17 | 126 | C45- | CAAATGCAGCTGGTCGAGTCTGGCGGTGGGGTAGTGCAAC |
| | | VH-L-VL | CAGGCCGTTCTCTGCGTCTTAGCTGCGCCGCATCTGGTTTT |
| | | | ACCTTTCGTACCTACGGTATGCACTGGGTGCGTCAGGCACC |
| | | | AGGCAAAGGTCTGGAATGGGTCGCAGTAATCTGGTATGAT |
| | | | GGTAGCAATAAACACTATGCTGACTCAGTCAAAGGCCGTTT |
| | | | CACCATCACCCGTGATAACAGCAAGAACACTCTTAACTTAC |
| | | | AGATGAACTCTCTGCGTGCCGAAGACACCGCCGTTTACTAC |
| | | | TGTGCCCGTGCACCACAGTGGGAATTAACCTCCGAAGCATT |
| | | | CGATCTTTGGGGCCAGGGCACTATGGTGACCGTTAGCTCTG |
| | | | GCGGTGGTGGTAGCGGAGGCGGAGGATCAGGTGGAGGCG |
| | | | GCAGTTCTTACGTGCTGACTCAACCACCATCAGTGTCTGTA |
| | | | GCACCAGGCCAGACCGCACGTATTACCTGTGGCGGTAACC |
| | | | TGATCGGCTCTAAGCTGGTTCACTGGTATCAGCAAAAACCA |
| | | | GGCCAGGCACCAGTACTGGTTGTGTACGATGATAGCGATC |
| | | | GTCCAAGCGAAATTCCAGAGCGTTTCAGCGGCTCTAATTCC |
| | | | GGCAACACCGCTACTCTGACTATTTCCCGTGTTGAAGCCGG |
| | | | CGATGAAGCCGACTACTATTGCCAGGTCTGGGACTCCAGCT |
| | | | CCGACGGTGTAGTCTTTGGCGGGGGCACCAAACTGACCGTT |
| | | | TTG |
| 18 | 127 | C7- | CAAATGCAGCTGGTCGAGTCTGGCGGTGGGGTAGTGCAAC |
| | | VH-L-VL | CAGGCCGTTCTCTGCGTCTTAGCTGCGCCGCATCTGGTTTT |
| | | | ACCTTTCGTACCTACGGTATGCACTGGGTGCGTCAGGCACC |
| | | | AGGCAAAGGTCTGGAATGGGTCGCAGTAATCTGGTATGAT |
| | | | GGTAGCAATAAACACTATGCTGACTCAGTCAAAGGCCGTTT |
| | | | CACCATCACCCGTGATAACAGCAAGAACACTCTTAACTTAC |
| | | | AGATGAACTCTCTGCGTGCCGAAGACACCGCCGTTTACTAC |
| | | | TGTGCCCGTGCACCACAGTGGCTGTTAGTAGCGGAAGCATT |
| | | | CGATCTCTGGGGCCAGGGCACTATGGTGACCGTTAGCTCTG |
| | | | GCGGTGGTGGTAGCGGAGGCGGAGGATCAGGTGGAGGCG |
| | | | GCAGTTCTTACGTGCTGACTCAACCACCATCAGTGTCTGTA |
| | | | GCACCAGGCCAGACCGCACGTATTACCTGTGGCGGTAACA |
| | | | TCATCGGCTCTAAGCTGGTTCACTGGTATCAGCAAAAACCA |
| | | | GGCCAGGCACCAGTACTGGTTGTGTACGATGATAGCGATC |
| | | | GTCCAAGCGGTATTCCAGAGCGTTTCAGCGGCTCTAATTCC |
| | | | GGCAACACCGCTACTCTGACTATTTCCCGTGTTGAAGCCGG |
| | | | CGATGAAGCCGACTACTATTGCCAGGTCTGGGACTCTGGCT |
| | | | CCGACCACGTAGTCTTTGGCGGGGGCACCAAACTGACCGTT |
| | | | TTG |
| 19 | 128 | C31- | CAAATGCAGCTGGTCGAGTCTGGCGGTGGGGTAGTGCAAC |
| | | VH-L-VL | CAGGCCGTTCTCTGCGTCTTAGCTGCGCCGCATCTGGTTTT |
| | | | ACCTTTCGTACCTACGGTATGCACTGGGTGCGTCAGGCACC |
| | | | AGGCAAAGGTCTGGAATGGGTCGCAGTAATCTGGTATGAT |
| | | | GGTAGCAATAAACACTATGCTGACTCAGTCAAAGGCCGTTT |
| | | | CACCATCACCCGTGATAACAGCAAGAACACTCTTAACTTAC |
| | | | AGATGAACTCTCTGCGTGCCGAAGACACCGCCGTTTACTAC |
| | | | TGTGCCCGTGCACCACAGTGGGAATTAGTAGCGGAAGCAT |
| | | | TCGATCTTTGGGGCCAGGGCACTATGGTGACCGTTAGCTCT |
| | | | GGCGGTGGTGGTAGCGGAGGCGGAGGATCAGGTGGAGGC |
| | | | GGCAGTTCTTACGTGCTGACTCAACCACCATCAGTGTCTGT |
| | | | AGCACCAGGCCAGACCGCACGTATTACCTGTGGCGGTAAC |
| | | | ATCATCGGCTCTAAGCTGGTTCACTGGTATCAGCAAAAACC |
| | | | AGGCCAGGCACCAGTACTGGTTGTGTACGATGATAGCGAT |
| | | | CGTCCAAGCGATATTCCAGAGCGTTTCAGCGGCTCTAATTC |
| | | | CGGCAACACCGCTACTCTGACTATTTCCCGTGTTGAAGCCG |
| | | | GCGATGAAGCCGACTACTATTGCCAGGTCTGGGACTCCGG |
| | | | CTCCGACGGTGTAGTCTTTGGCGGGGGCACCAAACTGACC |
| | | | GTTTTG |
| 20 | 129 | C15- | CAAATGCAGCTGGTCGAGTCTGGCGGTGGGGTAGTGCAAC |
| | | VH-L-VL | CAGGCCGTTCTCTGCGTCTTAGCTGCGCCGCATCTGGTTTT |
| | | | ACCTTTCGTACCTACGGTATGCACTGGGTGCGTCAGGCACC |
| | | | AGGCAAAGGTCTGGAATGGGTCGCAGTAATCTGGTATGAT |
| | | | GGTAGCAATAAACACTATGCTGACTCAGTCAAAGGCCGTTT |
| | | | CACCATCACCCGTGATAACAGCAAGAACACTCTTAACTTAC |
| | | | AGATGAACTCTCTGCGTGCCGAAGACACCGCCGTTTACTAC |
| | | | TGTGCCCGTGCACCACAGTGGGAATTAGTAGCGGAAGCAT |
| | | | TCGATCTGTGGGGCCAGGGCACTATGGTGACCGTTAGCTCT |
| | | | GGCGGTGGTGGTAGCGGAGGCGGAGGATCAGGTGGAGGC |
| | | | GGCAGTTCTTACGTGCTGACTCAACCACCATCAGTGTCTGT |
| | | | AGCACCAGGCCAGACCGCACGTATTACCTGTGGCGGTAAC |
| | | | CTGATCGGCTCTAAGCTGGTTCACTGGTATCAGCAAAAACC |
| | | | AGGCCAGGCACCAGTACTGGTTGTGTACGATGATGGCGAT |
| | | | CGTCCAAGCTGGATTCCAGAGCGTTTCAGCGGCTCTAATTC |
| | | | CGGCAACACCGCTACTCTGACTATTTCCCGTGTTGAAGCCG |
| | | | GCGATGAAGCCGACTACTATTGCCAGGTCTGGGACTCTGGC |
| | | | TCCGACGGTGTAGTCTTTGGCGGGGGCACCAAACTGACCGT |
| | | | TTTG |
| 21 | 130 | C35- | CAAATGCAGCTGGTCGAGTCTGGCGGTGGGGTAGTGCAAC |
| | | VH-L-VL | CAGGCCGTTCTCTGCGTCTTAGCTGCGCCGCATCTGGTTTT |
| | | | ACCTTTCGTACCTACGGTATGCACTGGGTGCGTCAGGCACC |
| | | | AGGCAAAGGTCTGGAATGGGTCGCAGTAATCTGGTATGAT |
| | | | GGTAGCAATAAACACTATGCTGACTCAGTCAAAGGCCGTTT |
| | | | CACCATCACCCGTGATAACAGCAAGAACACTCTTAACTTAC |
| | | | AGATGAACTCTCTGCGTGCCGAAGACACCGCCGTTTACTAC |
| | | | TGTGCCCGTGCACCACAGTGGGTCTTAGTATCCGAAGCATT |
| | | | CGATCTTTGGGGCCAGGGCACTATGGTGACCGTTAGCTCTG |
| | | | GCGGTGGTGGTAGCGGAGGCGGAGGATCAGGTGGAGGCG |
| | | | GCAGTTCTTACGTGCTGACTCAACCACCATCAGTGTCTGTA |
| | | | GCACCAGGCCAGACCGCACGTATTACCTGTGGCGGTAACC |
| | | | TGATCGGCTCTAAGCTGGTTCACTGGTATCAGCAAAAACCA |
| | | | GGCCAGGCACCAGTACTGGTTGTGTACGATGATAGCGATC |
| | | | GTCCAAGCCGTATTCCAGAGCGTTTCAGCGGCTCTAATTCC |
| | | | GGCAACACCGCTACTCTGACTATTTCCCGTGTTGAAGCCGG |
| | | | CGATGAAGCCGACTACTATTGCCAGGTCTGGGACTCCGGCT |
| | | | CCGACGGTGTAGTCTTTGGCGGGGGCACCAAACTGACCGTT |
| | | | TTG |
| 22 | 131 | C12- | CAAATGCAGCTGGTCGAGTCTGGCGGTGGGGTAGTGCAAC |
| | | VH-L-VL | CAGGCCGTTCTCTGCGTCTTAGCTGCGCCGCATCTGGTTTT |
| | | | ACCTTTCGTACCTACGGTATGCACTGGGTGCGTCAGGCACC |
| | | | AGGCAAAGGTCTGGAATGGGTCGCAGTAATCTGGTATGAT |
| | | | GGTAGCAATAAACACTATGCTGACTCAGTCAAAGGCCGTTT |
| | | | CACCATCACCCGTGATAACAGCAAGAACACTCTTAACTTAC |
| | | | AGATGAACTCTCTGCGTGCCGAAGACACCGCCGTTTACTAC |
| | | | TGTGCCCGTGCACCACAGTGGGAATTAGTAGCGGAGGCAT |
| | | | TCGATCTGTGGGGCCAGGGCACTATGGTGACCGTTAGCTCT |
| | | | GGCGGTGGTGGTAGCGGAGGCGGAGGATCAGGTGGAGGC |
| | | | GGCAGTTCTTACGTGCTGACTCAACCACCATCAGTGTCTGT |
| | | | AGCACCAGGCCAGACCGCACGTATTACCTGTGGCGGTAAC |
| | | | ATCATCGGCTCTAAGCTGGTTCACTGGTATCAGCAAAAACC |
| | | | AGGCCAGGCACCAGTACTGGTTGTGTACGATGATAGCGAT |
| | | | CGTCCAAGCGCAATTCCAGAGCGTTTCAGCGGCTCTAATTC |
| | | | CGGCAACACCGCTACTCTGACTATTTCCCGTGTTGAAGCCG |
| | | | GCGATGAAGCCGACTACTATTGCCAGGTCTGGGACTCCAG |
| | | | CTCCGACGGTGTAGTCTTTGGCGGGGGCACCAAACTGACC |
| | | | GTTTTG |
| 23 | 132 | C4- | CAAATGCAGCTGGTCGAGTCTGGCGGTGGGGTAGTGCAAC |
| | | VH-L-VL | CAGGCCGTTCTCTGCGTCTTAGCTGCGCCGCATCTGGTTTT |
| | | | ACCTTTCGTACCTACGGTATGCACTGGGTGCGTCAGGCACC |
| | | | AGGCAAAGGTCTGGAATGGGTCGCAGTAATCTCTTATGAT |
| | | | GGTAGCAATAAACACTATGCTGACTCAGTCAAAGGCCGTTT |
| | | | CACCATCACCCGTGATAACAGCAAGAACACTCTTAACTTAC |
| | | | AGATGAACTCTCTGCGTGCCGAAGACACCGCCGTTTACTAC |
| | | | TGTGCCCGTAGCCCACAGTGGGAATGGGTACACGAAGCAT |
| | | | TCGATATGTGGGGCCAGGGCACTATGGTGACCGTTAGCTCT |
| | | | GGCGGTGGTGGTAGCGGAGGCGGAGGATCAGGTGGAGGC |
| | | | GGCAGTTCTTACGTGCTGACTCAACCACCATCAGTGTCTGT |
| | | | AGCACCAGGCCAGACCGCACGTATTACCTGTGGCGGTAAC |
| | | | AACATCGGCTCTAAGCTGGTTCACTGGTATCAGCAAAAACC |
| | | | AGGCCAGGCACCAGTACTGGTTGTGTACGATGATAGCGAT |
| | | | CGTCCAAGCGGTATTCCAGAGCGTTTCAGCGGCTCTAATTC |
| | | | CGGCAACACCGCTACTCTGACTATTTCCCGTGTTGAAGCCG |
| | | | GCGATGAAGCCGACTACTATTGCCAGGTCTGGGACTCTAGC |
| | | | TCCGACGGTGTAGTCTTTGGCGGGGGCACCAAACTGACCGT |
| | | | TTTG |
| 24 | 133 | C41- | CAAATGCAGCTGGTCGAGTCTGGCGGTGGGGTAGTGCAAC |
| | | VH-L-VL | CAGGCCGTTCTCTGCGTCTTAGCTGCGCCGCATCTGGTTTT |
| | | | ACCTTTCGTACCTACGGTATGCACTGGGTGCGTCAGGCACC |
| | | | AGGCAAAGGTCTGGAATGGGTCGCAGTAATCTCTTATGAT |
| | | | GGTAGCAATAAACACTATGCTGACTCAGTCAAAGGCCGTTT |
| | | | CACCATCACCCGTGATAACAGCAAGAACACTCTTAACTTAC |
| | | | AGATGAACTCTCTGCGTGCCGAAGACACCGCCGTTTACTAC |
| | | | TGTGCCCGTTCGCCACAGTGGGAATGGGTACACGAAGCAT |
| | | | TCGATCTCTGGGGCCAGGGCACTATGGTGACCGTTAGCTCT |
| | | | GGCGGTGGTGGTAGCGGAGGCGGAGGATCAGGTGGAGGC |
| | | | GGCAGTTCTTACGTGCTGACTCAACCACCATCAGTGTCTGT |
| | | | AGCACCAGGCCAGACCGCACGTATTACCTGTGGCGGTAAC |
| | | | ATCCTGGGCTCTAAGCTGGTTCACTGGTATCAGCAAAAACC |
| | | | AGGCCAGGCACCAGTACTGGTTGTGTACGATGATAGCGAT |
| | | | CGTCCAAGCGAAATTCCAGAGCGTTTCAGCGGCTCTAATTC |
| | | | CGGCAACACCGCTACTCTGACTATTTCCCGTGTTGAAGCCG |
| | | | GCGATGAAGCCGACTACTATTGCCAGGTCTGGGACACCAG |
| | | | CTCCGACGGTGTAGTCTTTGGCGGGGGCACCAAACTGACC |
| | | | GTTTTG |
| 25 | 134 | C40- | CAAATGCAGCTGGTCGAGTCTGGCGGTGGGGTAGTGCAAC |
| | | VH-L-VL | CAGGCCGTTCTCTGCGTCTTAGCTGCGCCGCATCTGGTTTT |
| | | | ACCTTTCGTACCTACGGTATGCACTGGGTGCGTCAGGCACC |
| | | | AGGCAAAGGTCTGGAATGGGTCGCAGTAATCTGGTATGAT |
| | | | GGTAGCAATAAACACTATGCTGACTCAGTCAAAGGCCGTTT |
| | | | CACCATCACCCGTGATAACAGCAAGAACACTCTTAACTTAC |
| | | | AGATGAACTCTCTGCGTGCCGAAGACACCGCCGTTTACTAC |
| | | | TGTGCCCGTAGCCCACAGTGGGAATGGGTACACGAAGCAT |
| | | | TCGATCTATGGGGCCAGGGCACTATGGTGACCGTTAGCTCT |
| | | | GGCGGTGGTGGTAGCGGAGGCGGAGGATCAGGTGGAGGC |
| | | | GGCAGTTCTTACGTGCTGACTCAACCACCATCAGTGTCTGT |
| | | | AGCACCAGGCCAGACCGCACGTATTACCTGTGGCGGTAAC |
| | | | AACATCGGCTCTAAGCTGGTTCACTGGTATCAGCAAAAACC |
| | | | AGGCCAGGCACCAGTACTGGTTGTGTACGATGATAGCGAT |
| | | | CGTCCAAGCCGGATTCCAGAGCGTTTCAGCGGCTCTAATTC |
| | | | CGGCAACACCGCTACTCTGACTATTTCCCGTGTTGAAGCCG |
| | | | GCGATGAAGCCGACTACTATTGCCAGGTCTGGGACACTAG |
| | | | CTCCGACGGTGTAGTCTTTGGCGGGGGCACCAAACTGACC |
| | | | GTTTTG |
| 26 | 135 | C9- | CAAATGCAGCTGGTCGAGTCTGGCGGTGGGGTAGTGCAAC |
| | | VH-L-VL | CAGGCCGTTCTCTGCGTCTTAGCTGCGCCGCATCTGGTTTT |
| | | | ACCTTTCGTACCTACGGTATGCACTGGGTGCGTCAGGCACC |
| | | | AGGCAAAGGTCTGGAATGGGTCGCAGTAATCTGGTATGAT |
| | | | GGTAGCAATAAACACTATGCTGACTCAGTCAAAGGCCGTTT |
| | | | CACCATCACCCGTGATAACAGCAAGAACACTCTTAACTTAC |
| | | | AGATGAACTCTCTGCGTGCCGAAGACACCGCCGTTTACTAC |
| | | | TGTGCCCGTTCGCCACAGTGGGAATGGGTACACGAAGCAT |
| | | | TCGATCTCTGGGGCCAGGGCACTATGGTGACCGTTAGCTCT |
| | | | GGCGGTGGTGGTAGCGGAGGCGGAGGATCAGGTGGAGGC |
| | | | GGCAGTTCTTACGTGCTGACTCAACCACCATCAGTGTCTGT |
| | | | AGCACCAGGCCAGACCGCACGTATTACCTGTGGCGGTAAC |
| | | | CTGATCGGCTCTAAGCTGGTTCACTGGTATCAGCAAAAACC |
| | | | AGGCCAGGCACCAGTACTGGTTGTGTACGATGATGGCGAT |
| | | | CGTCCAAGCTGGATTCCAGAGCGTTTCAGCGGCTCTAATTC |
| | | | CGGCAACACCGCTACTCTGACTATTTCCCGTGTTGAAGCCG |
| | | | GCGATGAAGCCGACTACTATTGCCAGGTCTGGGACTCCAG |
| | | | CTCCGACGGTGTAGTCTTTGGCGGGGGCACCAAACTGACC |
| | | | GTTTTG |

TABLE 3
Nucleotide Sequences Encoding Engineered dual
binding antibodies: Variable Light chain (VL)
and Variable Heavy chain (VH) nucleic acid sequences

| | SEQ | | | SEQ | | |
|---|---|---|---|---|---|---|
| Ab | ID | | | ID | | |
| # | NO: | Name | VL Sequence | NO: | Name | VH Sequence |
| | 56 | Template- | TCTTACGTGCTGACTCA | 55 | Template- | CAAATGCAGCTGGTCGA |
| | | VL | ACCACCATCAGTGTCTG | | VH | GTCTGGCGGTGGGGTAG |
| | | | TAGCACCAGGCCAGAC | | | TGCAACCAGGCCGTTCT |
| | | | CGCACGTATTACCTGTG | | | CTGCGTCTTAGCTGCGC |
| | | | GCGGTAACAATCTGGG | | | CGCATCTGGTTTTACCTT |
| | | | CTCTAAGTCTGTTCACT | | | TCGTACCTACGGTATGC |
| | | | GGTATCAGCAAAAACC | | | ACTGGGTGCGTCAGGCA |
| | | | AGGCCAGGCACCAGTA | | | CCAGGCAAAGGTCTGGA |
| | | | CTGGTTGTGTACGATGA | | | ATGGGTCGCAGTAATCT |
| | | | TTCCGATCGTCCAAGCT | | | GGTATGATGGTAGCAAT |
| | | | GGATTCCAGAGCGTTTC | | | AAACACTATGCTGACTC |
| | | | AGCGGCTCTAATTCCGG | | | AGTCAAAGGCCGTTTCA |
| | | | CAACACCGCTACTCTGA | | | CCATCACCCGTGATAAC |
| | | | CTATTTCCCGTGGGGAA | | | AGCAAGAACACTCTTAA |
| | | | GCCGGCGATGAAGCCG | | | CTTACAGATGAACTCTC |
| | | | ACTACTATTGCCAGGTC | | | TGCGTGCCGAAGACACC |
| | | | TGGGACTCTTCTTCCGA | | | GCAGTTTATTACTGTGCC |
| | | | CCATGTAGTCTTTGGCG | | | CGTGCACCACAGTGGGA |
| | | | GGGGCACCAAACTGAC | | | ATTAGTACACGAAGCAT |
| | | | CGTTTTG | | | TCGATATCTGGGGTCAG |
| | | | | | | GGTACTATGGTGACCGT |
| | | | | | | TAGCTCT |
| 1 | 58 | C2- | TCTTACGTGCTGACTCA | 57 | C2- | CAAATGCAGCTGGTCGA |
| | | VL | ACCACCATCAGTGTCTG | | VH | GTCTGGCGGTGGGGTAG |
| | | | TAGCACCAGGCCAGAC | | | TGCAACCAGGCCGTTCT |
| | | | CGCACGTATTACCTGTG | | | CTGCGTCTTAGCTGCGC |
| | | | GCGGTAACCTGATCGG | | | CGCATCTGGTTTTACCTT |
| | | | CTCTAAGCTGGTTCACT | | | TCGTACCTACGGTATGC |
| | | | GGTATCAGCAAAAACC | | | ACTGGGTGCGTCAGGCA |
| | | | AGGCCAGGCACCAGTA | | | CCAGGCAAAGGTCTGGA |
| | | | CTGGTTGTGTACGATGA | | | ATGGGTCGCAGTAATCT |
| | | | TAGCGATCGTCCAAGCC | | | GGTATGATGGTAGCAAT |
| | | | TGATTCCAGAGCGTTTC | | | AAACACTATGCTGACTC |
| | | | AGCGGCTCTAATTCCGG | | | AGTCAAAGGCCGTTTCA |
| | | | CAACACCGCTACTCTGA | | | CCATCACCCGTGATAAC |
| | | | CTATTTCCCGTGTTGAA | | | AGCAAGAACACTCTTAA |
| | | | GCCGGCGATGAAGCCG | | | CTTACAGATGAACTCTC |
| | | | ACTACTATTGCCAGGTC | | | TGCGTGCCGAAGACACC |
| | | | TGGGACTCTAGCTCCGA | | | GCCGTTTACTACTGTGCC |
| | | | CGGTGTAGTCTTTGGCG | | | CGTGCACCACAGTGGGA |
| | | | GGGGCACCAAACTGAC | | | ATTAACGGCGGAAGCAT |
| | | | CGTTTTG | | | TCGATATTTGGGGCCAG |
| | | | | | | GGCACTATGGTGACCGT |
| | | | | | | TAGCTCT |
| 2 | 60 | C27- | TCTTACGTGCTGACTCA | 59 | C27- | CAAATGCAGCTGGTCGA |
| | | VL | ACCACCATCAGTGTCTG | | VH | GTCTGGCGGTGGGGTAG |
| | | | TAGCACCAGGCCAGAC | | | TGCAACCAGGCCGTTCT |
| | | | CGCACGTATTACCTGTG | | | CTGCGTCTTAGCTGCGC |
| | | | GCGGTAACCTGATCGG | | | CGCATCTGGTTTTACCTT |
| | | | CTCTAAGCTGGTTCACT | | | TCGTACCTACGGTATGC |
| | | | GGTATCAGCAAAAACC | | | ACTGGGTGCGTCAGGCA |
| | | | AGGCCAGGCACCAGTA | | | CCAGGCAAAGGTCTGGA |
| | | | CTGGTTGTGTACGATGA | | | ATGGGTCGCAGTAATCT |
| | | | TAGCGATCGTCCAAGCC | | | GGTATGATGGTAGCAAT |
| | | | TGATTCCAGAGCGTTTC | | | AAACACTAT |
| | | | AGCGGCTCTAATTCCGG | | | GCTGACTCAGTCAAAGG |
| | | | CAACACCGCTACTCTGA | | | CCGTTTCACCATCACCC |
| | | | CTATTTCCCGTGTTGAA | | | GTGATAACAGCAAGAAC |
| | | | GCCGGCGATGAAGCCG | | | ACTCTTAACTTACAGAT |
| | | | ACTACTATTGCCAGGTC | | | GAACTCTCTGCGTGCCG |
| | | | TGGGACTCTAGCTCCGA | | | AAGACACCGCCGTTTAC |
| | | | CGGTGTAGTCTTTGGCG | | | TACTGTGCCCGTGCACC |
| | | | GGGGCACCAAACTGAC | | | ACAGTGGGAATTAGTAG |
| | | | CGTTTTG | | | CGGAAGCATTCGATCTG |
| | | | | | | TGGGGCCAGGGCACTAT |
| | | | | | | GGTGACCGTTAGCTCT |
| 3 | 62 | C19- | TCTTACGTGCTGACTCA | 61 | C19- | CAAATGCAGCTGGTCGA |
| | | VL | ACCACCATCAGTGTCTG | | VH | GTCTGGCGGTGGGGTAG |
| | | | TAGCACCAGGCCAGAC | | | TGCAACCAGGCCGTTCT |
| | | | CGCACGTATTACCTGTG | | | CTGCGTCTTAGCTGCGC |
| | | | GCGGTAACCTGATCGG | | | CGCATCTGGTTTTACCTT |
| | | | CTCTAAGCTGGTTCACT | | | TCGTACCTACGGTATGC |
| | | | GGTATCAGCAAAAACC | | | ACTGGGTGCGTCAGGCA |
| | | | AGGCCAGGCACCAGTA | | | CCAGGCAAAGGTCTGGA |
| | | | CTGGTTGTGTACGATGA | | | ATGGGTCGCAGTAATCT |
| | | | TAGCGATCGTCCAAGCC | | | GGTATGATGGTAGCAAT |
| | | | TTATTCCAGAGCGTTTC | | | AAACACTATGCTGACTC |
| | | | AGCGGCTCTAATTCCGG | | | AGTCAAAGGCCGTTTCA |
| | | | CAACACCGCTACTCTGA | | | CCATCACCCGTGATAAC |
| | | | CTATTTCCCGTGTTGAA | | | AGCAAGAACACTCTTAA |
| | | | GCCGGCGATGAAGCCG | | | CTTACAGATGAACTCTC |
| | | | ACTACTATTGCCAGGTC | | | TGCGTGCCGAAGACACC |
| | | | TGGGACACCAGCTCCG | | | GCCGTTTACTACTGTGCC |
| | | | ACGGTGTAGTCTTTGGC | | | CGTGCACCACAGTGGCA |
| | | | GGGGGCACCAAACTGA | | | GTTAGTAGCGGAAGCAT |
| | | | CCGTTTTG | | | TCGATATTTGGGGCCAG |
| | | | | | | GGCACTATGGTGACCGT |
| | | | | | | TAGCTCT |
| 4 | 64 | C43- | TCTTACGTGCTGACTCA | 63 | C43- | CAAATGCAGCTGGTCGA |
| | | VL | ACCACCATCAGTGTCTG | | VH | GTCTGGCGGTGGGGTAG |
| | | | TAGCACCAGGCCAGAC | | | TGCAACCAGGCCGTTCT |
| | | | CGCACGTATTACCTGTG | | | CTGCGTCTTAGCTGCGC |
| | | | GCGGTAACCTGATCGG | | | CGCATCTGGTTTTACCTT |
| | | | CTCTAAGCTGGTTCACT | | | TCGTACCTACGGTATGC |
| | | | GGTATCAGCAAAAACC | | | ACTGGGTGCGTCAGGCA |
| | | | AGGCCAGGCACCAGTA | | | CCAGGCAAAGGTCTGGA |
| | | | CTGGTTGTGTACGATGA | | | ATGGGTCGCAGTAATCT |
| | | | TAGCGATCGTCCAAGC | | | GGTATGATGGTAGCAAT |
| | | | GCTATTCCAGAGCGTTT | | | AAACACTATGCTGACTC |
| | | | CAGCGGCTCTAATTCCG | | | AGTCAAAGGCCGTTTCA |
| | | | GCAACACCGCTACTCTG | | | CCATCACCCGTGATAAC |
| | | | ACTATTTCCCGTGTTGA | | | AGCAAGAACACTCTTAA |
| | | | AGCCGGCGATGAAGCC | | | CTTACAGATGAACTCTC |
| | | | GACTACTATTGCCAGGT | | | TGCGTGCCGAAGACACC |
| | | | CTGGGACACTAGCTCCG | | | GCCGTTTACTACTGTGCC |
| | | | ACGGTGTAGTCTTTGGC | | | CGTGCACCACAGTGGCA |
| | | | GGGGGCACCAAACTGA | | | GTTAGTAGCCGAAGCAT |
| | | | CCGTTTTG | | | TCGATATATGGGGCCAG |
| | | | | | | GGCACTATGGTGACCGT |
| | | | | | | TAGCTCT |
| 5 | 66 | C5- | TCTTACGTGCTGACTCA | 65 | C5- | CAAATGCAGCTGGTCGA |
| | | VL | ACCACCATCAGTGTCTG | | VH | GTCTGGCGGTGGGGTAG |
| | | | TAGCACCAGGCCAGAC | | | TGCAACCAGGCCGTTCT |
| | | | CGCACGTATTACCTGTG | | | CTGCGTCTTAGCTGCGC |
| | | | GCGGTAACCTGATCGG | | | CGCATCTGGTTTTACCTT |
| | | | CTCTAAGCTGGTTCACT | | | TCGTACCTACGGTATGC |
| | | | GGTATCAGCAAAAACC | | | ACTGGGTGCGTCAGGCA |
| | | | AGGCCAGGCACCAGTA | | | CCAGGCAAAGGTCTGGA |
| | | | CTGGTTGTGTACGATGA | | | ATGGGTCGCAGTAATCT |
| | | | TAGCGATCGTCCAAGCC | | | GGTATGATGGTAGCAAT |
| | | | TTATTCCAGAGCGTTTC | | | AAACACTATGCTGACTC |
| | | | AGCGGCTCTAATTCCGG | | | AGTCAAAGGCCGTTTCA |
| | | | CAACACCGCTACTCTGA | | | CCATCACCCGTGATAAC |
| | | | CTATTTCCCGTGTTGAA | | | AGCAAGAACACTCTTAA |
| | | | GCCGGCGATGAAGCCG | | | CTTACAGATGAACTCTC |
| | | | ACTACTATTGCCAGGTC | | | TGCGTGCCGAAGACACC |
| | | | TGGGACACCAGCTCCG | | | GCCGTTTACTACTGTGCC |
| | | | ACGGTGTAGTCTTTGGC | | | CGTGCACCACAGTGGGA |
| | | | GGGGGCACCAAACTGA | | | ATTAGTGGCGGAAGCAT |
| | | | CCGTTTTG | | | TCGATCTTTGGGGCCAG |
| | | | | | | GGCACTATGGTGACCGT |
| | | | | | | TAGCTCT |
| 6 | 68 | C8- | TCTTACGTGCTGACTCA | 67 | C8- | CAAATGCAGCTGGTCGA |
| | | VL | ACCACCATCAGTGTCTG | | VH | GTCTGGCGGTGGGGTAG |
| | | | TAGCACCAGGCCAGAC | | | TGCAACCAGGCCGTTCT |
| | | | CGCACGTATTACCTGTG | | | CTGCGTCTTAGCTGCGC |
| | | | GCGGTAACCTGCTGGG | | | CGCATCTGGTTTTACCTT |
| | | | CTCTAAGCTGGTTCACT | | | TCGTACCTACGGTATGC |
| | | | GGTATCAGCAAAAACC | | | ACTGGGTGCGTCAGGCA |
| | | | AGGCCAGGCACCAGTA | | | CCAGGCAAAGGTCTGGA |
| | | | CTGGTTGTGTACGATGA | | | ATGGGTCGCAGTAATCT |
| | | | TAGCGATCGTCCAAGC | | | GGTATGATGGTAGCAAT |
| | | | GCAATTCCAGAGCGTTT | | | AAACACTATGCTGACTC |
| | | | CAGCGGCTCTAATTCCG | | | AGTCAAAGGCCGTTTCA |
| | | | GCAACACCGCTACTCTG | | | CCATCACCCGTGATAAC |
| | | | ACTATTTCCCGTGTTGA | | | AGCAAGAACACTCTTAA |
| | | | AGCCGGCGATGAAGCC | | | CTTACAGATGAACTCTC |
| | | | GACTACTATTGCCAGGT | | | TGCGTGCCGAAGACACC |
| | | | CTGGGACTCTAGCTCCG | | | GCCGTTTACTACTGTGCC |
| | | | ACCACGTAGTCTTTGGC | | | CGTGCACCACAGTGGGA |
| | | | GGGGGCACCAAACTGA | | | ATTGGTAGCCGAAGCAT |
| | | | CCGTTTTG | | | TCGATATCTGGGGCCAG |
| | | | | | | GGCACTATGGTGACCGT |
| | | | | | | TAGCTCT |
| 7 | 70 | C6- | TCTTACGTGCTGACTCA | 69 | C6- | CAAATGCAGCTGGTCGA |
| | | VL | ACCACCATCAGTGTCTG | | VH | GTCTGGCGGTGGGGTAG |
| | | | TAGCACCAGGCCAGAC | | | TGCAACCAGGCCGTTCT |
| | | | CGCACGTATTACCTGTG | | | CTGCGTCTTAGCTGCGC |
| | | | GCGGTAACCTGATCGG | | | CGCATCTGGTTTTACCTT |
| | | | CTCTAAGCTGGTTCACT | | | TCGTACCTACGGTATGC |
| | | | GGTATCAGCAAAAACC | | | ACTGGGTGCGTCAGGCA |
| | | | AGGGCAGGCACCAGTA | | | CCAGGCAAAGGTCTGGA |
| | | | CTGGTTGTGTACGATGA | | | ATGGGTCGCAGTAATCT |
| | | | TAGCGATCGTCCAAGCC | | | GGTATGATGGTAGCAAT |
| | | | GGATTCCAGAGCGTTTC | | | AAACACTATGCTGACTC |
| | | | AGCGGCTCTAATTCCGG | | | AGTCAAAGGCCGTTTCA |
| | | | CAACACCGCTACTCTGA | | | CCATCACCCGTGATAAC |
| | | | CTATTTCCCGTGTTGAA | | | AGCAAGAACACTCTTAA |
| | | | GCCGGCGATGAAGCCG | | | CTTACAGATGAACTCTC |
| | | | ACTACTATTGCCAGGTC | | | TGCGTGCCGAAGACACC |
| | | | TGGGACTCCAGCTCCGA | | | GCCGTTTACTACTGTGCC |
| | | | CCATGTAGTCTTTGGCG | | | CGTGCACCACAGTGGAT |
| | | | GGGGCACCAAACTGAC | | | GTTAGTAGCGGAAGCAT |
| | | | CGTTTTG | | | TCGATCTATGGGGCCAG |
| | | | | | | GGCACTATGGTGACCGT |
| | | | | | | TAGCTCT |
| 8 | 72 | C3- | TCTTACGTGCTGACTCA | 71 | C3- | CAAATGCAGCTGGTCGA |
| | | VL | ACCACCATCAGTGTCTG | | VH | GTCTGGCGGTGGGGTAG |
| | | | TAGCACCAGGCCAGAC | | | TGCAACCAGGCCGTTCT |
| | | | CGCACGTATTACCTGTG | | | CTGCGTCTTAGCTGCGC |
| | | | GCGGTAACCTGATCGG | | | CGCATCTGGTTTTACCTT |
| | | | CTCTAAGCTGGTTCACT | | | TCGTACCTACGGTATGC |
| | | | GGTATCAGCAAAAACC | | | ACTGGGTGCGTCAGGCA |
| | | | AGGCCAGGCACCAGTA | | | CCAGGCAAAGGTCTGGA |
| | | | CTGGTTGTGTACGATGA | | | ATGGGTCGCAGTAATCT |
| | | | TAGCGATCGTCCAAGCC | | | GGTATGATGGTAGCAAT |
| | | | GGATTCCAGAGCGTTTC | | | AAACACTATGCTGACTC |
| | | | AGCGGCTCTAATTCCGG | | | AGTCAAAGGCCGTTTCA |
| | | | CAACACCGCTACTCTGA | | | CCATCACCCGTGATAAC |
| | | | CTATTTCCCGTGTTGAA | | | AGCAAGAACACTCTTAA |
| | | | GCCGGCGATGAAGCCG | | | CTTACAGATGAACTCTC |
| | | | ACTACTATTGCCAGGTC | | | TGCGTGCCGAAGACACC |
| | | | TGGGACTCCAGCTCCGA | | | GCCGTTTACTACTGTGCC |
| | | | CCACGTAGTCTTTGGCG | | | CGTGCACCACAGTGGGA |
| | | | GGGGCACCAAACTGAC | | | ATGGGTAGCCGAAGCAT |
| | | | CGTTTTG | | | TCGATCTGTGGGGCCAG |
| | | | | | | GGCACTATGGTGACCGT |
| | | | | | | TAGCTCT |
| 9 | 74 | C37- | TCTTACGTGCTGACTCA | 73 | C37- | CAAATGCAGCTGGTCGA |
| | | VL | ACCACCATCAGTGTCTG | | VH | GTCTGGCGGTGGGGTAG |
| | | | TAGCACCAGGCCAGAC | | | TGCAACCAGGCCGTTCT |
| | | | CGCACGTATTACCTGTG | | | CTGCGTCTTAGCTGCGC |
| | | | GCGGTAACCTGATCGG | | | CGCATCTGGTTTTACCTT |
| | | | CTCTAAGCTGGTTCACT | | | TCGTACCTACGGTATGC |
| | | | GGTATCAGCAAAAACC | | | ACTGGGTGCGTCAGGCA |
| | | | AGGCCAGGCACCAGTA | | | CCAGGCAAAGGTCTGGA |
| | | | CTGGTTGTGTACGATGA | | | ATGGGTCGCAGTAATCT |
| | | | TAGCGATCGTCCAAGCC | | | GGTATGATGGTAGCAAT |
| | | | GGATTCCAGAGCGTTTC | | | AAACACTATGCTGACTC |
| | | | AGCGGCTCTAATTCCGG | | | AGTCAAAGGCCGTTTCA |
| | | | CAACACCGCTACTCTGA | | | CCATCACCCGTGATAAC |
| | | | CTATTTCCCGTGTTGAA | | | AGCAAGAACACTCTTAA |
| | | | GCCGGCGATGAAGCCG | | | CTTACAGATGAACTCTC |
| | | | ACTACTATTGCCAGGTC | | | TGCGTGCCGAAGACACC |
| | | | TGGGACTCTAGCTCCGA | | | GCCGTTTACTACTGTGCC |
| | | | CCATGTAGTCTTTGGCG | | | CGTGCACCACAGTGGGA |
| | | | GGGGCACCAAACTGAC | | | ATTAGTAGCCGAGGCAT |
| | | | CGTTTTG | | | TCGATATGTGGGGCCAG |
| | | | | | | GGCACTATGGTGACCGT |
| | | | | | | TAGCTCT |
| 10 | 76 | C32- | TCTTACGTGCTGACTCA | 75 | C32- | CAAATGCAGCTGGTCGA |
| | | VL | ACCACCATCAGTGTCTG | | VH | GTCTGGCGGTGGGGTAG |
| | | | TAGCACCAGGCCAGAC | | | TGCAACCAGGCCGTTCT |
| | | | CGCACGTATTACCTGTG | | | CTGCGTCTTAGCTGCGC |
| | | | GCGGTAACCTGATCGG | | | CGCATCTGGTTTTACCTT |
| | | | CTCTAAGCTGGTTCACT | | | TCGTACCTACGGTATGC |
| | | | GGTATCAGCAAAAACC | | | ACTGGGTGCGTCAGGCA |
| | | | AGGCCAGGCACCAGTA | | | CCAGGCAAAGGTCTGGA |
| | | | CTGGTTGTGTACGATGA | | | ATGGGTCGCAGTAATCT |
| | | | TAGCGATCGTCCAAGC | | | GGTATGATGGTAGCAAT |
| | | | AAAATTCCAGAGCGTTT | | | AAACACTATGCTGACTC |
| | | | CAGCGGCTCTAATTCCG | | | AGTCAAAGGCCGTTTCA |
| | | | GCAACACCGCTACTCTG | | | CCATCACCCGTGATAAC |
| | | | ACTATTTCCCGTGTTGA | | | AGCAAGAACACTCTTAA |
| | | | AGCCGGCGATGAAGCC | | | CTTACAGATGAACTCTC |
| | | | GACTACTATTGCCAGGT | | | TGCGTGCCGAAGACACC |
| | | | CTGGGACTCCAGCTCCG | | | GCCGTTTACTACTGTGCC |
| | | | ACCATGTAGTCTTTGGC | | | CGTGCACCACAGTGGGA |
| | | | GGGGGCACCAAACTGA | | | ATTAGTAGCCGAAGCAT |
| | | | CCGTTTTG | | | TCGATCTGTGGGGCCAG |
| | | | | | | GGCACTATGGTGACCGT |
| | | | | | | TAGCTCT |
| 11 | 78 | C38- | TCTTACGTGCTGACTCA | 77 | C38- | CAAATGCAGCTGGTCGA |
| | | VL | ACCACCATCAGTGTCTG | | VH | GTCTGGCGGTGGGGTAG |
| | | | TAGCACCAGGCCAGAC | | | TGCAACCAGGCCGTTCT |
| | | | CGCACGTATTACCTGTG | | | CTGCGTCTTAGCTGCGC |
| | | | GCGGTAACCTGATCGG | | | CGCATCTGGTTTTACCTT |
| | | | CTCTAAGCTGGTTCACT | | | TCGTACCTACGGTATGC |
| | | | GGTATCAGCAAAAACC | | | ACTGGGTGCGTCAGGCA |
| | | | AGGCCAGGCACCAGTA | | | CCAGGCAAAGGTCTGGA |
| | | | CTGGTTGTGTACGATGA | | | ATGGGTCGCAGTAATCT |
| | | | TAGCGATCGTCCAAGC | | | GGTATGATGGTAGCAAT |
| | | | GATATTCCAGAGCGTTT | | | AAACACTATGCTGACTC |
| | | | CAGCGGCTCTAATTCCG | | | AGTCAAAGGCCGTTTCA |
| | | | GCAACACCGCTACTCTG | | | CCATCACCCGTGATAAC |
| | | | ACTATTTCCCGTGTTGA | | | AGCAAGAACACTCTTAA |
| | | | AGCCGGCGATGAAGCC | | | CTTACAGATGAACTCTC |
| | | | GACTACTATTGCCAGGT | | | TGCGTGCCGAAGACACC |
| | | | CTGGGACACCAGCTCC | | | GCCGTTTACTACTGTGCC |
| | | | GACCATGTAGTCTTTGG | | | CGTGCACCACAGTGGGA |
| | | | CGGGGGCACCAAACTG | | | ATACGTAGCGGAAGCAT |
| | | | ACCGTTTTG | | | TCGATCTGTGGGGCCAG |
| | | | | | | GGCACTATGGTGACCGT |
| | | | | | | TAGCTCT |
| 12 | 80 | C26- | TCTTACGTGCTGACTCA | 79 | C26- | CAAATGCAGCTGGTCGA |
| | | VL | ACCACCATCAGTGTCTG | | VH | GTCTGGCGGTGGGGTAG |
| | | | TAGCACCAGGCCAGAC | | | TGCAACCAGGCCGTTCT |
| | | | CGCACGTATTACCTGTG | | | CTGCGTCTTAGCTGCGC |
| | | | GCGGTAACATCATCGG | | | CGCATCTGGTTTTACCTT |
| | | | CTCTAAGCTGGTTCACT | | | TCGTACCTACGGTATGC |
| | | | GGTATCAGCAAAAACC | | | ACTGGGTGCGTCAGGCA |
| | | | AGGCCAGGCACCAGTA | | | CCAGGCAAAGGTCTGGA |
| | | | CTGGTTGTGTACGATGA | | | ATGGGTCGCAGTAATCT |
| | | | TGGCGATCGTCCAAGC | | | GGTATGATGGTAGCAAT |
| | | | GGTATTCCAGAGCGTTT | | | AAACACTATGCTGACTC |
| | | | CAGCGGCTCTAATTCCG | | | AGTCAAAGGCCGTTTCA |
| | | | GCAACACCGCTACTCTG | | | CCATCACCCGTGATAAC |
| | | | ACTATTTCCCGTGTTGA | | | AGCAAGAACACTCTTAA |
| | | | AGCCGGCGATGAAGCC | | | CTTACAGATGAACTCTC |
| | | | GACTACTATTGCCAGGT | | | TGCGTGCCGAAGACACC |
| | | | CTGGGACACTAGCTCCG | | | GCCGTTTACTACTGTGCC |
| | | | ACCACGTAGTCTTTGGC | | | CGTGCACCACAGTGGGA |
| | | | GGGGGCACCAAACTGA | | | ATTAACGGCCGAAGCAT |
| | | | CCGTTTTG | | | TCGATCTTTGGGGCCAG |
| | | | | | | GGCACTATGGTGACCGT |
| | | | | | | TAGCTCT |
| 13 | 82 | C13- | TCTTACGTGCTGACTCA | 81 | C13- | CAAATGCAGCTGGTCGA |
| | | VL | ACCACCATCAGTGTCTG | | VH | GTCTGGCGGTGGGGTAG |
| | | | TAGCACCAGGCCAGAC | | | TGCAACCAGGCCGTTCT |
| | | | CGCACGTATTACCTGTG | | | CTGCGTCTTAGCTGCGC |
| | | | GCGGTAACCTGCTGGG | | | CGCATCTGGTTTTACCTT |
| | | | CTCTAAGCTGGTTCACT | | | TCGTACCTACGGTATGC |
| | | | GGTATCAGCAAAAACC | | | ACTGGGTGCGTCAGGCA |
| | | | AGGCCAGGCACCAGTA | | | CCAGGCAAAGGTCTGGA |
| | | | CTGGTTGTGTACGATGA | | | ATGGGTCGCAGTAATCT |
| | | | TGGCGATCGTCCAAGCC | | | GGTATGATGGTAGCAAT |
| | | | TGATTCCAGAGCGTTTC | | | AAACACTATGCTGACTC |
| | | | AGCGGCTCTAATTCCGG | | | AGTCAAAGGCCGTTTCA |
| | | | CAACACCGCTACTCTGA | | | CCATCACCCGTGATAAC |
| | | | CTATTTCCCGTGTTGAA | | | AGCAAGAACACTCTTAA |
| | | | GCCGGCGATGAAGCCG | | | CTTACAGATGAACTCTC |
| | | | ACTACTATTGCCAGGTC | | | TGCGTGCCGAAGACACC |
| | | | TGGGACACTAGCTCCG | | | GCCGTTTACTACTGTGCC |
| | | | ACCACGTAGTCTTTGGC | | | CGTGCACCACAGTGGGA |
| | | | GGGGGCACCAAACTGA | | | ATTAGTGGCGGAAGCAT |
| | | | CCGTTTTG | | | TCGATCTTTGGGGCCAG |
| | | | | | | GGCACTATGGTGACCGT |
| | | | | | | TAGCTCT |
| 14 | 84 | C23- | TCTTACGTGCTGACTCA | 83 | C23- | CAAATGCAGCTGGTCGA |
| | | VL | ACCACCATCAGTGTCTG | | VH | GTCTGGCGGTGGGGTAG |
| | | | TAGCACCAGGCCAGAC | | | TGCAACCAGGCCGTTCT |
| | | | CGCACGTATTACCTGTG | | | CTGCGTCTTAGCTGCGC |
| | | | GCGGTAACCTGCTGGG | | | CGCATCTGGTTTTACCTT |
| | | | CTCTAAGCTGGTTCACT | | | TCGTACCTACGGTATGC |
| | | | GGTATCAGCAAAAACC | | | ACTGGGTGCGTCAGGCA |
| | | | AGGCCAGGCACCAGTA | | | CCAGGCAAAGGTCTGGA |
| | | | CTGGTTGTGTACGATGA | | | ATGGGTCGCAGTAATCT |
| | | | TAGCGATCGTCCAAGC | | | GGTATGATGGTAGCAAT |
| | | | GAAATTCCAGAGCGTTT | | | AAACACTATGCTGACTC |
| | | | CAGCGGCTCTAATTCCG | | | AGTCAAAGGCCGTTTCA |
| | | | GCAACACCGCTACTCTG | | | CCATCACCCGTGATAAC |
| | | | ACTATTTCCCGTGTTGA | | | AGCAAGAACACTCTTAA |
| | | | AGCCGGCGATGAAGCC | | | CTTACAGATGAACTCTC |
| | | | GACTACTATTGCCAGGT | | | TGCGTGCCGAAGACACC |
| | | | CTGGGACACCAGCTCC | | | GCCGTTTACTACTGTGCC |
| | | | GACGGTGTAGTCTTTGG | | | CGTGCACCACAGTGGGA |
| | | | CGGGGGCACCAAACTG | | | ATTAACGGCGGAAGCAT |
| | | | ACCGTTTTG | | | TCGATATTTGGGGCCAG |
| | | | | | | GGCACTATGGTGACCGT |
| | | | | | | TAGCTCT |
| 15 | 86 | C39- | TCTTACGTGCTGACTCA | 85 | C39- | CAAATGCAGCTGGTCGA |
| | | VL | ACCACCATCAGTGTCTG | | VH | GTCTGGCGGTGGGGTAG |
| | | | TAGCACCAGGCCAGAC | | | TGCAACCAGGCCGTTCT |
| | | | CGCACGTATTACCTGTG | | | CTGCGTCTTAGCTGCGC |
| | | | GCGGTAACCTGCTGGG | | | CGCATCTGGTTTTACCTT |
| | | | CTCTAAGCTGGTTCACT | | | TCGTACCTACGGTATGC |
| | | | GGTATCAGCAAAAACC | | | ACTGGGTGCGTCAGGCA |
| | | | AGGCCAGGCACCAGTA | | | CCAGGCAAAGGTCTGGA |
| | | | CTGGTTGTGTACGATGA | | | ATGGGTCGCAGTAATCT |
| | | | TAGCGATCGTCCAAGCT | | | GGTATGATGGTAGCAAT |
| | | | GGATTCCAGAGCGTTTC | | | AAACACTATGCTGACTC |
| | | | AGCGGCTCTAATTCCGG | | | AGTCAAAGGCCGTTTCA |
| | | | CAACACCGCTACTCTGA | | | CCATCACCCGTGATAAC |
| | | | CTATTTCCCGTGTTGAA | | | AGCAAGAACACTCTTAA |
| | | | GCCGGCGATGAAGCCG | | | CTTACAGATGAACTCTC |
| | | | ACTACTATTGCCAGGTC | | | TGCGTGCCGAAGACACC |
| | | | TGGGACACTAGCTCCG | | | GCCGTTTACTACTGTGCC |
| | | | ACCATGTAGTCTTTGGC | | | CGTGCACCACAGTGGGA |
| | | | GGGGGCACCAAACTGA | | | ATTAACGGCCGAAGCAT |
| | | | CCGTTTTG | | | TCGATCTTTGGGGCCAG |
| | | | | | | GGCACTATGGTGACCGT |
| | | | | | | TAGCTCT |
| 16 | 88 | C17- | TCTTACGTGCTGACTCA | 87 | C17- | CAAATGCAGCTGGTCGA |
| | | VL | ACCACCATCAGTGTCTG | | VH | GTCTGGCGGTGGGGTAG |
| | | | TAGCACCAGGCCAGAC | | | TGCAACCAGGCCGTTCT |
| | | | CGCACGTATTACCTGTG | | | CTGCGTCTTAGCTGCGC |
| | | | GCGGTAACCTGATCGG | | | CGCATCTGGTTTTACCTT |
| | | | CTCTAAGCTGGTTCACT | | | TCGTACCTACGGTATGC |
| | | | GGTATCAGCAAAAACC | | | ACTGGGTGCGTCAGGCA |
| | | | AGGCCAGGCACCAGTA | | | CCAGGCAAAGGTCTGGA |
| | | | CTGGTTGTGTACGATGA | | | ATGGGTCGCAGTAATCT |
| | | | TAGCGATCGTCCAAGC | | | GGTATGATGGTAGCAAT |
| | | | GCAATTCCAGAGCGTTT | | | AAACACTATGCTGACTC |
| | | | CAGCGGCTCTAATTCCG | | | AGTCAAAGGCCGTTTCA |
| | | | GCAACACCGCTACTCTG | | | CCATCACCCGTGATAAC |
| | | | ACTATTTCCCGTGTTGA | | | AGCAAGAACACTCTTAA |
| | | | AGCCGGCGATGAAGCC | | | CTTACAGATGAACTCTC |
| | | | GACTACTATTGCCAGGT | | | TGCGTGCCGAAGACACC |
| | | | CTGGGACACCAGCTCC | | | GCCGTTTACTACTGTGCC |
| | | | GACCACGTAGTCTTTGG | | | CGTGCACCACAGTGGGA |
| | | | CGGGGGCACCAAACTG | | | ATTAACGTCGGAAGCAT |
| | | | ACCGTTTTG | | | TCGATCTTTGGGGCCAG |
| | | | | | | GGCACTATGGTGACCGT |
| | | | | | | TAGCTCT |
| 17 | 90 | C45- | TCTTACGTGCTGACTCA | 89 | C45- | CAAATGCAGCTGGTCGA |
| | | VL | ACCACCATCAGTGTCTG | | VH | GTCTGGCGGTGGGGTAG |
| | | | TAGCACCAGGCCAGAC | | | TGCAACCAGGCCGTTCT |
| | | | CGCACGTATTACCTGTG | | | CTGCGTCTTAGCTGCGC |
| | | | GCGGTAACCTGATCGG | | | CGCATCTGGTTTTACCTT |
| | | | CTCTAAGCTGGTTCACT | | | TCGTACCTACGGTATGC |
| | | | GGTATCAGCAAAAACC | | | ACTGGGTGCGTCAGGCA |
| | | | AGGCCAGGCACCAGTA | | | CCAGGCAAAGGTCTGGA |
| | | | CTGGTTGTGTACGATGA | | | ATGGGTCGCAGTAATCT |
| | | | TAGCGATCGTCCAAGC | | | GGTATGATGGTAGCAAT |
| | | | GAAATTCCAGAGCGTTT | | | AAACACTATGCTGACTC |
| | | | CAGCGGCTCTAATTCCG | | | AGTCAAAGGCCGTTTCA |
| | | | GCAACACCGCTACTCTG | | | CCATCACCCGTGATAAC |
| | | | ACTATTTCCCGTGTTGA | | | AGCAAGAACACTCTTAA |
| | | | AGCCGGCGATGAAGCC | | | CTTACAGATGAACTCTC |
| | | | GACTACTATTGCCAGGT | | | TGCGTGCCGAAGACACC |
| | | | CTGGGACTCCAGCTCCG | | | GCCGTTTACTACTGTGCC |
| | | | ACGGTGTAGTCTTTGGC | | | CGTGCACCACAGTGGGA |
| | | | GGGGGCACCAAACTGA | | | ATTAACCTCCGAAGCAT |
| | | | CCGTTTTG | | | TCGATCTTTGGGGCCAG |
| | | | | | | GGCACTATGGTGACCGT |
| | | | | | | TAGCTCT |
| 18 | 92 | C7- | TCTTACGTGCTGACTCA | 91 | C7- | CAAATGCAGCTGGTCGA |
| | | VL | ACCACCATCAGTGTCTG | | VH | GTCTGGCGGTGGGGTAG |
| | | | TAGCACCAGGCCAGAC | | | TGCAACCAGGCCGTTCT |
| | | | CGCACGTATTACCTGTG | | | CTGCGTCTTAGCTGCGC |
| | | | GCGGTAACATCATCGG | | | CGCATCTGGTTTTACCTT |
| | | | CTCTAAGCTGGTTCACT | | | TCGTACCTACGGTATGC |
| | | | GGTATCAGCAAAAACC | | | ACTGGGTGCGTCAGGCA |
| | | | AGGCCAGGCACCAGTA | | | CCAGGCAAAGGTCTGGA |
| | | | CTGGTTGTGTACGATGA | | | ATGGGTCGCAGTAATCT |
| | | | TAGCGATCGTCCAAGC | | | GGTATGATGGTAGCAAT |
| | | | GGTATTCCAGAGCGTTT | | | AAACACTATGCTGACTC |
| | | | CAGCGGCTCTAATTCCG | | | AGTCAAAGGCCGTTTCA |
| | | | GCAACACCGCTACTCTG | | | CCATCACCCGTGATAAC |
| | | | ACTATTTCCCGTGTTGA | | | AGCAAGAACACTCTTAA |
| | | | AGCCGGCGATGAAGCC | | | CTTACAGATGAACTCTC |
| | | | GACTACTATTGCCAGGT | | | TGCGTGCCGAAGACACC |
| | | | CTGGGACTCTGGCTCCG | | | GCCGTTTACTACTGTGCC |
| | | | ACCACGTAGTCTTTGGC | | | CGTGCACCACAGTGGCT |
| | | | GGGGGCACCAAACTGA | | | GTTAGTAGCGGAAGCAT |
| | | | CCGTTTTG | | | TCGATCTCTGGGGCCAG |
| | | | | | | GGCACTATGGTGACCGT |
| | | | | | | TAGCTCT |
| 19 | 94 | C31- | TCTTACGTGCTGACTCA | 93 | C31- | CAAATGCAGCTGGTCGA |
| | | VL | ACCACCATCAGTGTCTG | | VH | GTCTGGCGGTGGGGTAG |
| | | | TAGCACCAGGCCAGAC | | | TGCAACCAGGCCGTTCT |
| | | | CGCACGTATTACCTGTG | | | CTGCGTCTTAGCTGCGC |
| | | | GCGGTAACATCATCGG | | | CGCATCTGGTTTTACCTT |
| | | | CTCTAAGCTGGTTCACT | | | TCGTACCTACGGTATGC |
| | | | GGTATCAGCAAAAACC | | | ACTGGGTGCGTCAGGCA |
| | | | AGGCCAGGCACCAGTA | | | CCAGGCAAAGGTCTGGA |
| | | | CTGGTTGTGTACGATGA | | | ATGGGTCGCAGTAATCT |
| | | | TAGCGATCGTCCAAGC | | | GGTATGATGGTAGCAAT |
| | | | GATATTCCAGAGCGTTT | | | AAACACTATGCTGACTC |
| | | | CAGCGGCTCTAATTCCG | | | AGTCAAAGGCCGTTTCA |
| | | | GCAACACCGCTACTCTG | | | CCATCACCCGTGATAAC |
| | | | ACTATTTCCCGTGTTGA | | | AGCAAGAACACTCTTAA |
| | | | AGCCGGCGATGAAGCC | | | CTTACAGATGAACTCTC |
| | | | GACTACTATTGCCAGGT | | | TGCGTGCCGAAGACACC |
| | | | CTGGGACTCCGGCTCCG | | | GCCGTTTACTACTGTGCC |
| | | | ACGGTGTAGTCTTTGGC | | | CGTGCACCACAGTGGGA |
| | | | GGGGGCACCAAACTGA | | | ATTAGTAGCGGAAGCAT |
| | | | CCGTTTTG | | | TCGATCTTTGGGGCCAG |
| | | | | | | GGCACTATGGTGACCGT |
| | | | | | | TAGCTCT |
| 20 | 96 | C15- | TCTTACGTGCTGACTCA | 95 | C15- | CAAATGCAGCTGGTCGA |
| | | VL | ACCACCATCAGTGTCTG | | VH | GTCTGGCGGTGGGGTAG |
| | | | TAGCACCAGGCCAGAC | | | TGCAACCAGGCCGTTCT |
| | | | CGCACGTATTACCTGTG | | | CTGCGTCTTAGCTGCGC |
| | | | GCGGTAACCTGATCGG | | | CGCATCTGGTTTTACCTT |
| | | | CTCTAAGCTGGTTCACT | | | TCGTACCTACGGTATGC |
| | | | GGTATCAGCAAAAACC | | | ACTGGGTGCGTCAGGCA |
| | | | AGGCCAGGCACCAGTA | | | CCAGGCAAAGGTCTGGA |
| | | | CTGGTTGTGTACGATGA | | | ATGGGTCGCAGTAATCT |
| | | | TGGCGATCGTCCAAGCT | | | GGTATGATGGTAGCAAT |
| | | | GGATTCCAGAGCGTTTC | | | AAACACTATGCTGACTC |
| | | | AGCGGCTCTAATTCCGG | | | AGTCAAAGGCCGTTTCA |
| | | | CAACACCGCTACTCTGA | | | CCATCACCCGTGATAAC |
| | | | CTATTTCCCGTGTTGAA | | | AGCAAGAACACTCTTAA |
| | | | GCCGGCGATGAAGCCG | | | CTTACAGATGAACTCTC |
| | | | ACTACTATTGCCAGGTC | | | TGCGTGCCGAAGACACC |
| | | | TGGGACTCTGGCTCCGA | | | GCCGTTTACTACTGTGCC |
| | | | CGGTGTAGTCTTTGGCG | | | CGTGCACCACAGTGGGA |
| | | | GGGGCACCAAACTGAC | | | ATTAGTAGCGGAAGCAT |
| | | | CGTTTTG | | | TCGATCTGTGGGGCCAG |
| | | | | | | GGCACTATGGTGACCGT |
| | | | | | | TAGCTCT |
| 21 | 98 | C35- | TCTTACGTGCTGACTCA | 97 | C35- | CAAATGCAGCTGGTCGA |
| | | VL | ACCACCATCAGTGTCTG | | VH | GTCTGGCGGTGGGGTAG |
| | | | TAGCACCAGGCCAGAC | | | TGCAACCAGGCCGTTCT |
| | | | CGCACGTATTACCTGTG | | | CTGCGTCTTAGCTGCGC |
| | | | GCGGTAACCTGATCGG | | | CGCATCTGGTTTTACCTT |
| | | | CTCTAAGCTGGTTCACT | | | TCGTACCTACGGTATGC |
| | | | GGTATCAGCAAAAACC | | | ACTGGGTGCGTCAGGCA |
| | | | AGGCCAGGCACCAGTA | | | CCAGGCAAAGGTCTGGA |
| | | | CTGGTTGTGTACGATGA | | | ATGGGTCGCAGTAATCT |
| | | | TAGCGATCGTCCAAGCC | | | GGTATGATGGTAGCAAT |
| | | | GTATTCCAGAGCGTTTC | | | AAACACTATGCTGACTC |
| | | | AGCGGCTCTAATTCCGG | | | AGTCAAAGGCCGTTTCA |
| | | | CAACACCGCTACTCTGA | | | CCATCACCCGTGATAAC |
| | | | CTATTTCCCGTGTTGAA | | | AGCAAGAACACTCTTAA |
| | | | GCCGGCGATGAAGCCG | | | CTTACAGATGAACTCTC |
| | | | ACTACTATTGCCAGGTC | | | TGCGTGCCGAAGACACC |
| | | | TGGGACTCCGGCTCCGA | | | GCCGTTTACTACTGTGCC |
| | | | CGGTGTAGTCTTTGGCG | | | CGTGCACCACAGTGGGT |
| | | | GGGGCACCAAACTGAC | | | CTTAGTATCCGAAGCAT |
| | | | CGTTTTG | | | TCGATCTTTGGGGCCAG |
| | | | | | | GGCACTATGGTGACCGT |
| | | | | | | TAGCTCT |
| 22 | 100 | C12- | TCTTACGTGCTGACTCA | 99 | C12- | CAAATGCAGCTGGTCGA |
| | | VL | ACCACCATCAGTGTCTG | | VH | GTCTGGCGGTGGGGTAG |
| | | | TAGCACCAGGCCAGAC | | | TGCAACCAGGCCGTTCT |
| | | | CGCACGTATTACCTGTG | | | CTGCGTCTTAGCTGCGC |
| | | | GCGGTAACATCATCGG | | | CGCATCTGGTTTTACCTT |
| | | | CTCTAAGCTGGTTCACT | | | TCGTACCTACGGTATGC |
| | | | GGTATCAGCAAAAACC | | | ACTGGGTGCGTCAGGCA |
| | | | AGGCCAGGCACCAGTA | | | CCAGGCAAAGGTCTGGA |
| | | | CTGGTTGTGTACGATGA | | | ATGGGTCGCAGTAATCT |
| | | | TAGCGATCGTCCAAGC | | | GGTATGATGGTAGCAAT |
| | | | GCAATTCCAGAGCGTTT | | | AAACACTATGCTGACTC |
| | | | CAGCGGCTCTAATTCCG | | | AGTCAAAGGCCGTTTCA |
| | | | GCAACACCGCTACTCTG | | | CCATCACCCGTGATAAC |
| | | | ACTATTTCCCGTGTTGA | | | AGCAAGAACACTCTTAA |
| | | | AGCCGGCGATGAAGCC | | | CTTACAGATGAACTCTC |
| | | | GACTACTATTGCCAGGT | | | TGCGTGCCGAAGACACC |
| | | | CTGGGACTCCAGCTCCG | | | GCCGTTTACTACTGTGCC |
| | | | ACGGTGTAGTCTTTGGC | | | CGTGCACCACAGTGGGA |
| | | | GGGGGCACCAAACTGA | | | ATTAGTAGCGGAGGCAT |
| | | | CCGTTTTG | | | TCGATCTGTGGGGCCAG |
| | | | | | | GGCACTATGGTGACCGT |
| | | | | | | TAGCTCT |
| 23 | 102 | C4- | TCTTACGTGCTGACTCA | 101 | C4- | CAAATGCAGCTGGTCGA |
| | | VL | ACCACCATCAGTGTCTG | | VH | GTCTGGCGGTGGGGTAG |
| | | | TAGCACCAGGCCAGAC | | | TGCAACCAGGCCGTTCT |
| | | | CGCACGTATTACCTGTG | | | CTGCGTCTTAGCTGCGC |
| | | | GCGGTAACAACATCGG | | | CGCATCTGGTTTTACCTT |
| | | | CTCTAAGCTGGTTCACT | | | TCGTACCTACGGTATGC |
| | | | GGTATCAGCAAAAACC | | | ACTGGGTGCGTCAGGCA |
| | | | AGGCCAGGCACCAGTA | | | CCAGGCAAAGGTCTGGA |
| | | | CTGGTTGTGTACGATGA | | | ATGGGTCGCAGTAATCT |
| | | | TAGCGATCGTCCAAGC | | | CTTATGATGGTAGCAAT |
| | | | GGTATTCCAGAGCGTTT | | | AAACACTATGCTGACTC |
| | | | CAGCGGCTCTAATTCCG | | | AGTCAAAGGCCGTTTCA |
| | | | GCAACACCGCTACTCTG | | | CCATCACCCGTGATAAC |
| | | | ACTATTTCCCGTGTTGA | | | AGCAAGAACACTCTTAA |
| | | | AGCCGGCGATGAAGCC | | | CTTACAGATGAACTCTC |
| | | | GACTACTATTGCCAGGT | | | TGCGTGCCGAAGACACC |
| | | | CTGGGACTCTAGCTCCG | | | GCCGTTTACTACTGTGCC |
| | | | ACGGTGTAGTCTTTGGC | | | CGTAGCCCACAGTGGGA |
| | | | GGGGGCACCAAACTGA | | | ATGGGTACACGAAGCAT |
| | | | CCGTTTTG | | | TCGATATGTGGGGCCAG |
| | | | | | | GGCACTATGGTGACCGT |
| | | | | | | TAGCTCT |
| 24 | 104 | C41- | TCTTACGTGCTGACTCA | 103 | C41- | CAAATGCAGCTGGTCGA |
| | | VL | ACCACCATCAGTGTCTG | | VH | GTCTGGCGGTGGGGTAG |
| | | | TAGCACCAGGCCAGAC | | | TGCAACCAGGCCGTTCT |
| | | | CGCACGTATTACCTGTG | | | CTGCGTCTTAGCTGCGC |
| | | | GCGGTAACATCCTGGG | | | CGCATCTGGTTTTACCTT |
| | | | CTCTAAGCTGGTTCACT | | | TCGTACCTACGGTATGC |
| | | | GGTATCAGCAAAAACC | | | ACTGGGTGCGTCAGGCA |
| | | | AGGCCAGGCACCAGTA | | | CCAGGCAAAGGTCTGGA |
| | | | CTGGTTGTGTACGATGA | | | ATGGGTCGCAGTAATCT |
| | | | TAGCGATCGTCCAAGC | | | CTTATGATGGTAGCAAT |
| | | | GAAATTCCAGAGCGTTT | | | AAACACTATGCTGACTC |
| | | | CAGCGGCTCTAATTCCG | | | AGTCAAAGGCCGTTTCA |
| | | | GCAACACCGCTACTCTG | | | CCATCACCCGTGATAAC |
| | | | ACTATTTCCCGTGTTGA | | | AGCAAGAACACTCTTAA |
| | | | AGCCGGCGATGAAGCC | | | CTTACAGATGAACTCTC |
| | | | GACTACTATTGCCAGGT | | | TGCGTGCCGAAGACACC |
| | | | CTGGGACACCAGCTCC | | | GCCGTTTACTACTGTGCC |
| | | | GACGGTGTAGTCTTTGG | | | CGTTCGCCACAGTGGGA |
| | | | CGGGGGCACCAAACTG | | | ATGGGTACACGAAGCAT |
| | | | ACCGTTTTG | | | TCGATCTCTGGGGCCAG |
| | | | | | | GGCACTATGGTGACCGT |
| | | | | | | TAGCTCT |
| 25 | 106 | C40- | TCTTACGTGCTGACTCA | 105 | C40- | CAAATGCAGCTGGTCGA |
| | | VL | ACCACCATCAGTGTCTG | | VH | GTCTGGCGGTGGGGTAG |
| | | | TAGCACCAGGCCAGAC | | | TGCAACCAGGCCGTTCT |
| | | | CGCACGTATTACCTGTG | | | CTGCGTCTTAGCTGCGC |
| | | | GCGGTAACAACATCGG | | | CGCATCTGGTTTTACCTT |
| | | | CTCTAAGCTGGTTCACT | | | TCGTACCTACGGTATGC |
| | | | GGTATCAGCAAAAACC | | | ACTGGGTGCGTCAGGCA |
| | | | AGGCCAGGCACCAGTA | | | CCAGGCAAAGGTCTGGA |
| | | | CTGGTTGTGTACGATGA | | | ATGGGTCGCAGTAATCT |
| | | | TAGCGATCGTCCAAGCC | | | GGTATGATGGTAGCAAT |
| | | | GGATTCCAGAGCGTTTC | | | AAACACTATGCTGACTC |
| | | | AGCGGCTCTAATTCCGG | | | AGTCAAAGGCCGTTTCA |
| | | | CAACACCGCTACTCTGA | | | CCATCACCCGTGATAAC |
| | | | CTATTTCCCGTGTTGAA | | | AGCAAGAACACTCTTAA |
| | | | GCCGGCGATGAAGCCG | | | CTTACAGATGAACTCTC |
| | | | ACTACTATTGCCAGGTC | | | TGCGTGCCGAAGACACC |
| | | | TGGGACACTAGCTCCG | | | GCCGTTTACTACTGTGCC |
| | | | ACGGTGTAGTCTTTGGC | | | CGTAGCCCACAGTGGGA |
| | | | GGGGGCACCAAACTGA | | | ATGGGTACACGAAGCAT |
| | | | CCGTTTTG | | | TCGATCTATGGGGCCAG |
| | | | | | | GGCACTATGGTGACCGT |
| | | | | | | TAGCTCT |
| 26 | 108 | C9- | TCTTACGTGCTGACTCA | 107 | C9- | CAAATGCAGCTGGTCGA |
| | | VL | ACCACCATCAGTGTCTG | | VH | GTCTGGCGGTGGGGTAG |
| | | | TAGCACCAGGCCAGAC | | | TGCAACCAGGCCGTTCT |
| | | | CGCACGTATTACCTGTG | | | CTGCGTCTTAGCTGCGC |
| | | | GCGGTAACCTGATCGG | | | CGCATCTGGTTTTACCTT |
| | | | CTCTAAGCTGGTTCACT | | | TCGTACCTACGGTATGC |
| | | | GGTATCAGCAAAAACC | | | ACTGGGTGCGTCAGGCA |
| | | | AGGCCAGGCACCAGTA | | | CCAGGCAAAGGTCTGGA |
| | | | CTGGTTGTGTACGATGA | | | ATGGGTCGCAGTAATCT |
| | | | TGGCGATCGTCCAAGCT | | | GGTATGATGGTAGCAAT |
| | | | GGATTCCAGAGCGTTTC | | | AAACACTATGCTGACTC |
| | | | AGCGGCTCTAATTCCGG | | | AGTCAAAGGCCGTTTCA |
| | | | CAACACCGCTACTCTGA | | | CCATCACCCGTGATAAC |
| | | | CTATTTCCCGTGTTGAA | | | AGCAAGAACACTCTTAA |
| | | | GCCGGCGATGAAGCCG | | | CTTACAGATGAACTCTC |
| | | | ACTACTATTGCCAGGTC | | | TGCGTGCCGAAGACACC |
| | | | TGGGACTCCAGCTCCGA | | | GCCGTTTACTACTGTGCC |
| | | | CGGTGTAGTCTTTGGCG | | | CGTTCGCCACAGTGGGA |
| | | | GGGGCACCAAACTGAC | | | ATGGGTACACGAAGCAT |
| | | | CGTTTTG | | | TCGATCTCTGGGGCCAG |
| | | | | | | GGCACTATGGTGACCGT |
| | | | | | | TAGCTCT |
| | 162 | 33.023 | TCTTACGTGCTGACTCA | 161 | 33.023 | CAAATGCAGCTGGTCGA |
| | | VL | ACCACCATCAGTGTCTG | | VH | GTCTGGCGGTGGGGTAG |
| | | | TAGCACCAGGCCAGAC | | | TGCAACCAGGCCGTTCT |
| | | | CGCACGTATTACCTGTG | | | CTGCGTCTTAGCTGCGC |
| | | | GCGGTAACCTGATCGG | | | CGCATCTGGTTTTGCGTT |
| | | | CTCTAAGCTGGTTCACT | | | TCGTACCTACGGTATGC |
| | | | GGTATCAGCAAAAACC | | | ACTGGGTGCGTCAGGCA |
| | | | AGGCCAGGCACCAGTA | | | CCAGGCAAAGGTCTGGA |
| | | | CTGGTTGTGTACGATGA | | | ATGGGTCGCAGTAATCT |
| | | | TAGCGATCGTCCAAGCC | | | GGTATGATGGTAGCAAT |
| | | | GTATTCCAGAGCGTTTC | | | ACCCACTATGCTGACTC |
| | | | AGCGGCTCTAATTCCGG | | | AGTCAAAGGCCGTTTCA |
| | | | CAACACCGCTACTCTGA | | | CCATCACCCGTGATAAC |
| | | | CTATTTCCCGTGTTGAA | | | AGCAAGAACACTCTTAA |
| | | | GCCGGCGATGAAGCCG | | | CTTACAGATGAACTCTC |
| | | | ACTACTATTGCCAGGTC | | | TGCGTGCCGAAGACACC |
| | | | TGGGACCACAGCTCCG | | | GCCGTTTACTACTGTGCC |
| | | | ACCATGTAGTCTTTGGC | | | CGTGCACCACAGTGGTA |
| | | | GGGGGCACCAAACTGA | | | CTTAAGCGCGGAAGCAT |
| | | | CCGTTTTG | | | TCGATCTATGGGGCCAG |
| | | | | | | GGCACTATGGTGACCGT |
| | | | | | | TAGCTCT |
| | 164 | 33.025 | TCTTACGTGCTGACTCA | 163 | 33.025 | CAAATGCAGCTGGTCGA |
| | | VL | ACCACCATCAGTGTCTG | | VH | GTCTGGCGGTGGGGTAG |
| | | | TAGCACCAGGCCAGAC | | | TGCAACCAGGCCGTTCT |
| | | | CGCACGTATTACCTGTG | | | CTGCGTCTTAGCTGCGC |
| | | | GCGGTAACCTGATCGG | | | CGCATCTGGTTTTACCTT |
| | | | CTCTAAGCTGGTTCACT | | | TCGTACCTACGGTATGC |
| | | | GGTATCAGCAAAAACC | | | ACTGGGTGCGTCAGGCA |
| | | | AGGCCAGGCACCAGTA | | | CCAGGCAAAGGTCTGGA |
| | | | CTGGTTGTGTACGATGA | | | ATGGGTCGCAGACATCT |
| | | | TAGCGATCGTCCAAGCC | | | GGTATGATGGTAGCAAT |
| | | | TGATTCCAGAGCGTTTC | | | AAACACTATGCTGACTC |
| | | | AGCGGCTCTAATTCCGG | | | AGTCAAAGGCCGTTTCA |
| | | | CAACACCGCTACTCTGA | | | CCATCACCCGTGATAAC |
| | | | CTATTTCCCGTGTTGAA | | | AGCAAGAACACTCTTAA |
| | | | GCCGGCGATGAAGCCG | | | CTTACAGATGAACTCTC |
| | | | ACTACTATTGCCAGGTC | | | TGCGTGCCGAAGACACC |
| | | | TGGGACTCTAGCTCCGA | | | GCCGTTTACTACTGTGCC |
| | | | CGGTGTAGTCTTTGGCG | | | CGTGCACCACAGTGGTA |
| | | | GGGGCACCAAACTGAC | | | CTTAGTAGCGGAACCGT |
| | | | CGTTTTG | | | TCGATCTATGGGGCCAG |
| | | | | | | GGCACTATGGTGACCGT |
| | | | | | | TAGCTCT |
| | 184 | 38.014 | AGTTACGTGCTGACACA | 183 | 38.014 | CAGATGCAGTTGGTGGA |
| | | VL | ACCTCCAAGTGTTAGTG | | VH | GTCCGGAGGTGGAGTGG |
| | | | TCGCACCAGGACAAAC | | | TGCAACCAGGGCGTTCC |
| | | | AGCACGTATTACATGTG | | | TTGCGTTTGTCTTGTGCT |
| | | | GAGGAAATCTTATCGGT | | | GCTTCCGGATTCGCCTTT |
| | | | GCCAAGCTGGTGCACT | | | CGTACATATGGCATGCA |
| | | | GGTACCAGCAGAAACC | | | TTGGGTGCGTCAAGCAC |
| | | | TGGTCAGGCCCCAGTAC | | | CTGGTAAGGGCCTGGAG |
| | | | TGGTTGTGTATGATGAC | | | TGGGTTGCCGTTATTTGG |
| | | | AGCGACCGTCCAAGCC | | | TACGACGGCTCCAACAC |
| | | | GTATCCCAGAACGTTTT | | | CCACTACGCAGATAGCG |
| | | | TCTGGGAGCAACTCAG | | | TGAAAGGACGTTTCACT |
| | | | GTAATACAGCCACTCTG | | | ATTACCCGTGATAACTC |
| | | | ACCATTTCACGTGTTGA | | | CAAGAATACCCTTAACC |
| | | | GGCAGGAGATGAGGCC | | | TGCAGATGAATAGCTTG |
| | | | GATTATTATTGCCAAGT | | | CGTGCTGAGGACACAGC |
| | | | ATGGGACCACAGCTCT | | | AGTATATTACTGCGTCC |
| | | | GACCATGTTGTTTTTGG | | | GTGCACCACAATGGTAC |
| | | | CGGAGGGACTAAGCTG | | | CTGAGCGCCGAGGCCTT |
| | | | ACCGTGCTT | | | TGATCTGTGGGGGCAGG |
| | | | | | | GCACAATGGTGACCGTT |
| | | | | | | TCCTCA |
| | 186 | 38.018 | TCCTATGTGCTGACACA | 185 | 38.018 | CAGATGCAACTGGTGGA |
| | | VL | GCCACCTAGCGTGAGC | | VH | GTCAGGAGGCGGCGTGG |
| | | | GTCGCCCCAGGTCAGA | | | TGCAGCCAGGACGTTCT |
| | | | CCGCTCGTATCACTTGT | | | CTGCGTCTGTCTTGCGCA |
| | | | GGCGGGAACCTTATCG | | | GCTTCCGGGTTCGCCTTT |
| | | | GCAGCAAGCTGGTGCA | | | CGTACCTATGGGATGCA |
| | | | CTGGTACCAGCAGAAG | | | TTGGGTGCGTCAGGCTC |
| | | | CCTGGCCAAGCACCTGT | | | CAGGTAAGGGACTGGAG |
| | | | GCTGGTCGTTTATGACG | | | TGGGTCGCTGTTATTTGG |
| | | | ACTCTGACCGTCCATCC | | | GACGACGGAAGTAACAC |
| | | | CGTATCCCAGAACGTTT | | | TCATTACGCCGACAGCG |
| | | | CTCTGGCTCTAACTCTG | | | TGAAGGGCCGTTTCACA |
| | | | GGAATACCGCTACCCTG | | | ATTACCCGTGACAATTC |
| | | | ACAATCTCACGTGTTGA | | | CAAGAATACCTTGAACC |
| | | | AGCTGGCGACGAGGCA | | | TGCAGATGAACTCTCTT |
| | | | GATTATTATTGCCAAGT | | | CGTGCTGAAGATACCGC |
| | | | CTGGGATCACTCCAGCG | | | CGTGTACTATTGCGCCC |
| | | | ATCACGTCGTGTTCGGA | | | GTGCTCCACAGTGGTAT |
| | | | GGCGGAACAAAATTGA | | | CTGTCAGCAGAGGCCTT |
| | | | CTGTCCTG | | | CGACCTGTGGGGACAGG |
| | | | | | | GAACAATGGTGACCGTA |
| | | | | | | TCTTCA |
| | 188 | 38.019 | TCTTACGTGTTGACACA | 187 | 38.019 | CAAATGCAGCTGGTGGA |
| | | VL | ACCACCAAGTGTTAGTG | | VH | ATCCGGGGGTGGGGTCG |
| | | | TCGCACCTGGCCAAACC | | | TCCAGCCTGGCCGTAGT |
| | | | GCTCGTATCACCTGTGG | | | CTGCGTCTTTCCTGTGCC |
| | | | TGGGAATCTTATTGGCT | | | GCATCAGGCTTTGCTTTC |
| | | | CTAAGCTGGTGCACTGG | | | CGTACCTACGGGATGCA |
| | | | TATCAGCAGAAACCAG | | | CTGGGTGCGTCAGGCCC |
| | | | GCCAGGCTCCAGTACTG | | | CAGGAAAGGGACTTGAA |
| | | | GTGGTGTACGACGACTC | | | TGGGTGGCTGTCATCTG |
| | | | TGACCGTCCAAGCCGTA | | | GTACGATGGTTCCAACA |
| | | | TCCCAGAGCGTTTCAGT | | | CACACTATGCCGATTCA |
| | | | GGCTCTAACTCCGGGA | | | GTGAAAGGGCGTTTCAC |
| | | | ACACAGCAACTCTTACA | | | CATTACTCGTGACAATA |
| | | | ATTTCACGTGTGGAGGC | | | GTAAGAATACTCTGAAT |
| | | | CGGTGATGAAGCCGAC | | | CTGCAAATGAATTCACT |
| | | | TACTATTGCCAGGTTTG | | | GCGTGCTGAGGACACCG |
| | | | GGACTACAGTAGTAAT | | | CTGTTTATTACTGTGTGC |
| | | | CACGTGGTTTTCGGTGG | | | GTGCTCCTCAGTGGTAC |
| | | | TGGTACCAAGCTGACTG | | | CTGAGTGCCGAAGCTTT |
| | | | TGTTG | | | CGATTTGTGGGGACAGG |
| | | | | | | GCACAATGGTGACAGTC |
| | | | | | | AGTTCT |
| | 190 | 38.021 | AGCTATGTTCTGACTCA | 189 | 38.021 | CAGATGCAGCTGGTTGA |
| | | VL | ACCACCTAGTGTGAGTG | | VH | ATCTGGCGGCGGTGTGG |
| | | | TGGCCCCTGGTCAGACT | | | TCCAGCCTGGTCGTAGC |
| | | | GCACGTATTACCTGTGG | | | CTGCGTCTGTCCTGTGCT |
| | | | CGGAAACCTTATCGGC | | | GCAAGCGGATTTGCCTT |
| | | | AGTAAGCTGGTTCATTG | | | TGACACCTATGGGATGC |
| | | | GTATCAGCAGAAGCCA | | | ACTGGGTACGTCAGGCC |
| | | | GGACAGGCACCAGTGC | | | CCAGGAAAGGGCCTGGA |
| | | | TGGTCGTTTACGACGAT | | | ATGGGTGGCAGTTATCT |
| | | | AGTGACCGTCCATCACG | | | GGTATGATGGTTCTAAT |
| | | | TATCCCAGAGCGTTTTA | | | ACCGTGTATGCCGACTC |
| | | | GCGGGTCCAATTCCGG | | | CGTTAAAGGCCGTTTCA |
| | | | AAATACAGCAACCTTG | | | CTATCACCCGTGATAAT |
| | | | ACCATTAGCCGTGTGGA | | | AGTAAAAACACACTGAA |
| | | | AGCCGGCGATGAAGCT | | | CCTGCAGATGAATAGCT |
| | | | GATTATTACTGCCAGGT | | | TGCGTGCTGAGGACACC |
| | | | ATGGGACCATTCCTCCG | | | GCAGTGTACTACTGTGC |
| | | | ACCACTACGTTTTTGGT | | | CCGTGCTCCTCAGTGGT |
| | | | GGCGGAACTAAGCTGA | | | ATCTGTCAGCAGAGGCC |
| | | | CAGTCTTG | | | TTCGATCTGTGGGGCCA |
| | | | | | | AGGGACAATGGTGACCG |
| | | | | | | TGTCTTCC |
| | 192 | 38.025 | TCTTACGTGCTTACTCA | 191 | 38.025 | CAGATGCAGCTTGTTGA |
| | | VL | GCCTCCTAGCGTCTCAG | | VH | GAGCGGCGGAGGCGTGG |
| | | | TGGCCCCAGGCGAGAC | | | TGCAACCAGGCCGTTCA |
| | | | AGCAACCATTACATGC | | | TTGCGTCTGTCCTGCGCC |
| | | | GGGGGTAATTTGATCG | | | GCCAGCGGCTTTGCTTTT |
| | | | GTAGCAAGCTGGTGCA | | | CGTACATACGGCATGCA |
| | | | TTGGTATCAGCAGAAG | | | CTGGGTGCGTCAGGCCC |
| | | | CCTGGCCAGGCCCCAGT | | | CTGGCAAGGGGCTGGAA |
| | | | GCTGGTTGTATATGACG | | | TGGGTCGCCGTGATTTG |
| | | | ATAGTGATCGTCCAAGT | | | GTATGACGGTAGTAACA |
| | | | CGTATCCCTGAGCGTTT | | | CCCATTATGCTGATTCCG |
| | | | TAGCGGATCTAACTCCG | | | TCAAGGGACGTTTCACT |
| | | | GCAACACAGCCACATT | | | ATCACCCGTGACAATAG |
| | | | GACAATCAGCCGTGTG | | | CAAAAATACACTGAATC |
| | | | GAGGCAGGCGATGAGG | | | TGCAAATGAATTCATTG |
| | | | CCGACTACTACTGCCAA | | | CGTGCCGAAGACACCGC |
| | | | GTTTGGGACCACTCCTC | | | CGTATATTACTGTGTCCG |
| | | | TGACCACGTGGTATTTG | | | TGCCCCACAGTGGTACC |
| | | | GCGGAGGAACAAAGCT | | | TGAGCGCTGAGGCCTTC |
| | | | TACAGTTTTG | | | GATCTGTGGGGTCAGGG |
| | | | | | | GACTATGGTGACCGTAT |
| | | | | | | CATCC |
| | 194 | 38.026 | TCTTATGTTTTGACCCA | 193 | 38.026 | CAGATGCAGCTGGTGGA |
| | | VL | ACCTCCATCCGTTAGCG | | VH | GAGTGGAGGTGGTGTGG |
| | | | TGGCTCCAGGTCAAAC | | | TGCAACCTGGGCGTAGC |
| | | | AGCTACCATCACATGTG | | | CTGCGTTTGAGCTGCGC |
| | | | GCGGTAACCTTATTGGC | | | TGCCTCTGGATTTGCCTT |
| | | | TCAAAGCTGGTTCATTG | | | CCGTACCTATGGCATGC |
| | | | GTATCAACAGAAACCA | | | ACTGGGTGCGTCAGGCT |
| | | | GGCCAAGCCCCAGTGC | | | CCAGGAAAGGGGTTGGA |
| | | | TGGTGGTGTATGACGAC | | | ATGGGTGGCTGTGATTT |
| | | | AGTGACCGTCCTTCTCG | | | GGTACGACGGGAGCGCC |
| | | | TATTCCTGAGCGTTTTT | | | ACACATTACGCAGACAG |
| | | | CCGGCTCTAATATTGGC | | | CGTTAAGGGCCGTTTCA |
| | | | AACACTGCCACCCTGAC | | | CAATTACCCGTGACAAT |
| | | | CATTTCTCGTGTGGAAG | | | AGCAAAAATACATTGAA |
| | | | CAGGAGATGAGGCAGA | | | CCTGCAGATGAATTCCC |
| | | | CTATTATTGTCAGGTTT | | | TGCGTGCAGAGGATACT |
| | | | GGGATCACTCCAGCGA | | | GCAGTGTACTATTGCGT |
| | | | TCATGTGGTATTCGGAG | | | CCGTGCCCCACAGTGGT |
| | | | GTGGGACAAAACTTAC | | | ATCTGTCAGCCGAAGCC |
| | | | TGTTCTT | | | TTCGATCTGTGGGGGCA |
| | | | | | | GGGTACTATGGTCACCG |
| | | | | | | TAAGTTCC |
| | 196 | 38.040 | AGCTACGTGCTTACCCA | 195 | 38.040 | CAGATGCAGCTGGTGGA |
| | | VL | GCCACCATCAGTCAGTG | | VH | AAGTGGTGGGGGAGTCG |
| | | | TGGCTCCAGGCCAAACT | | | TGCAACCAGGACGTTCC |
| | | | GCCCGTATCACCTGCGG | | | TTGCGTCTGTCATGCGCT |
| | | | CGGCAATTTGATTGGCA | | | GCTTCAGGTTTCGACTTT |
| | | | CCAAGCTTGTGCACTGG | | | CGTACCTACGGCATGCA |
| | | | TACCAACAGAAGCCAG | | | TTGGGTGCGTCAGGCTC |
| | | | GGCAGGCCCCTGTGCTG | | | CAGGTAAAGGACTTGAG |
| | | | GTTGTCTACGACGATAG | | | TGGGTCGCAGTGATCTG |
| | | | TGATCGTCCTTCCCGTA | | | GTACGACGGATCAATTA |
| | | | TTCCTGAACGTTTCTCT | | | CTCACTACGCCGATAGC |
| | | | GGAAGCAATTCCGGAA | | | GTGAAAGGCCGTTTCAC |
| | | | ACACAGCCACACTTACC | | | CATCACCCGTGACAACT |
| | | | ATTTCTCGTGTTGAGGC | | | CCAAGAACACCCTGAAC |
| | | | TGGGGATGAAGCCGAC | | | TTGCAGATGAACAGTCT |
| | | | TACTATTGCCAGGTTTG | | | GCGTGCAGAAGACACTG |
| | | | GGACCACAATGAAGAC | | | CAGTATATTATTGTGTCC |
| | | | GAAGTTGTTTTTGGAGG | | | GTGCCCCACAGTGGTAC |
| | | | AGGAACTAAGCTGACA | | | TTGACCGCCGAGGCTTT |
| | | | GTTCTG | | | TGATCTGTGGGGACAGG |
| | | | | | | GCACAATGGTGACCGTA |
| | | | | | | TCTAGC |

TABLE 4
Amino Acid Sequences of Heavy-chain
CDR Regions for Antibody Clone #s.
33.023, 33.025, 38.014, 38.015. 38.018,
38.019, 38.021, 38.026, 38.040

| | | SEQ | HCDR2 | SEQ | | SEQ |
|---|---|---|---|---|---|---|
| CLONE | HCDR1 | ID | | ID | HCDR3 | ID |
| BDG33_023_VH | GFAFRTYG | 149 | IWYDGSNT | 150 | ARAPQWYLSAEAFDL | 151 |
| BDG33_025_VH | GFTFRTYG | 165 | IWYDGSNK | 166 | ARAPQWYLVAEPFDL | 167 |
| BDG38_014_VH | GFAFRTYG | 149 | IWYDGSNT | 150 | VRAPQWYLSAEAFDL | 201 |
| BDG38_018_VH | GFAFRTYG | 149 | IWDDGSNT | 208 | ARAPQWYLSAEAFDL | 151 |
| BDG38_019_VH | GFAFRTYG | 149 | IWYDGSNT | 150 | VRAPQWYLSAEAFDL | 201 |
| BDG38_021_VH | GFAFDTYG | 197 | IWYDGSNT | 150 | ARAPQWYLSAEAFDL | 151 |
| BDG38_025_VH | GFAFRTYG | 149 | IWYDGSNT | 150 | VRAPQWYLSAEAFDL | 201 |
| BDG38_026_VH | GFAFRTYG | 149 | IWYDGSAT | 199 | VRAPQWYLSAEAFDL | 201 |
| BDG38_040_VH | GFDFRTYG | 198 | IWYDGSIT | 200 | VRAPQWYLTAEAFDL | 202 |

TABLE 5
Amino Acid Sequences of Light-chain
CDR Regions for Antibody Clone #s.
33.023, 33.025, 38.014, 38.015. 38.018,
38.019, 38.021, 38.026, 38.040

| | | SEQ | | | SEQ |
|---|---|---|---|---|---|
| CLONE | LCDR1 | ID | LCDR2 | LCDR3 | ID |
| BDG33_023_VL | LIGSKL | 152 | DDS | QVWDHSSDHVV | 154 |
| BDG33_025_VL | LIGSKL | 152 | DDS | QVWDSSSDGVV | 168 |
| BDG38_014_VL | LIGAKL | 203 | DDS | QVWDHSSDHVV | 154 |
| BDG38_018_VL | LIGSKL | 152 | DDS | QVWDHSSDHVV | 154 |
| BDG38_019_VL | LIGSKL | 152 | DDS | QVWDYSSNHVV | 205 |
| BDG38_021_VL | LIGSKL | 152 | DDS | QVWDHSSDHYV | 206 |
| BDG38_025_VL | LIGSKL | 152 | DDS | QVWDHSSDHVV | 154 |
| BDG38_026_VL | LIGSKL | 152 | DDS | QVWDHSSDHVV | 154 |
| BDG38_040_VL | LIGTKL | 204 | DDS | QVWDHNEDEVV | 207 |

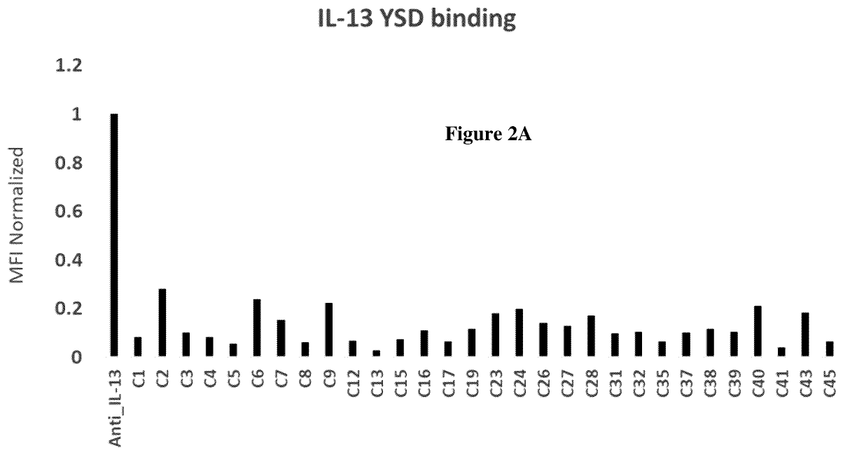

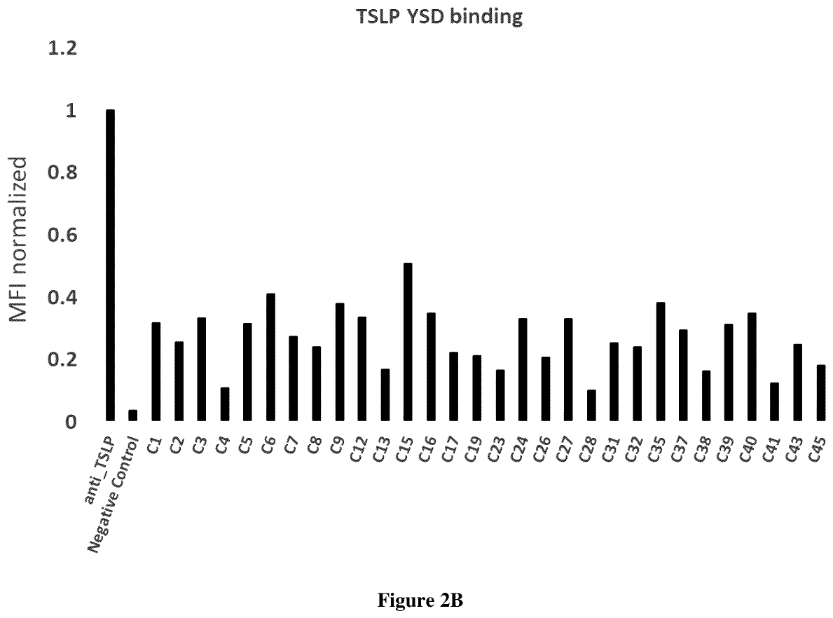

The clones were tested for their binding to 10 nM rh-IL-13 in yeast scFv format and were compared to a positive rh-IL-13 binder displayed on yeast as well. The affinity was normalized based on the mean fluorescence values of the positive control (normalized MFI). Relative affinity of the isolated clones for rh-IL-13 was between 3% and 30% of the affinity displayed by the positive control (FIG. 2A). C2, C6, C9, and C40 clones that exhibited above 20% of the relative affinity for rh-IL-13 and were shown to bind 10 nM TSLP in YSD (FIG. 2B) were chosen to be expressed as human IgG1.

### Example 3: Ab Production And Biochemical Characterization

Objective: To reformat the selected clones to a human IgG1 format and analyze the IgG1 antibodies for dual IL-13 and TSLP binding.

Results: Subsequent to characterization in the yeast surface display format described in Example 2, the selected clones C2, C6, and C9, were reformatted to human IgG1 by subcloning the variable domain into two separate expression vectors, pSF-CMV-HuIgG1_HC and pSF-CMV-HuLambda_LC, as described in Example 1 (Methods).

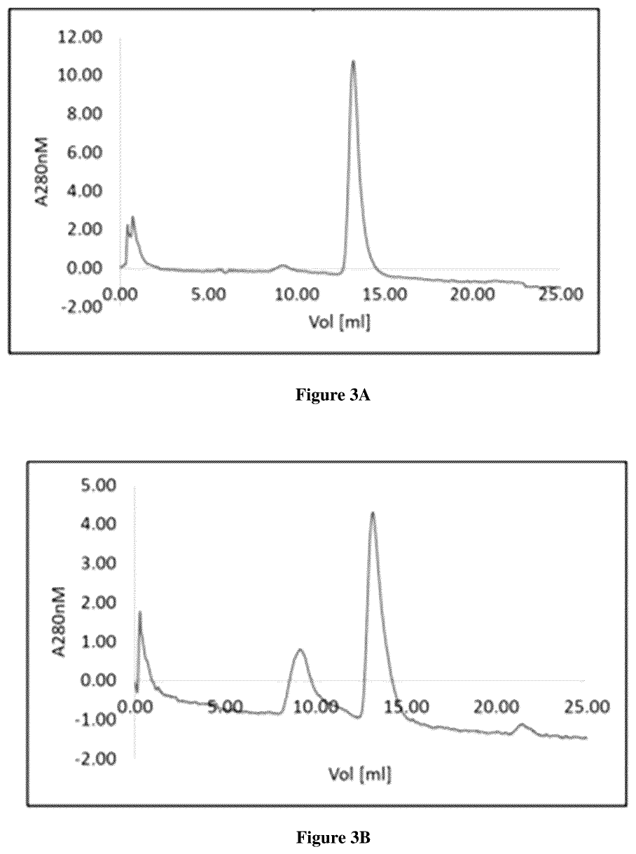

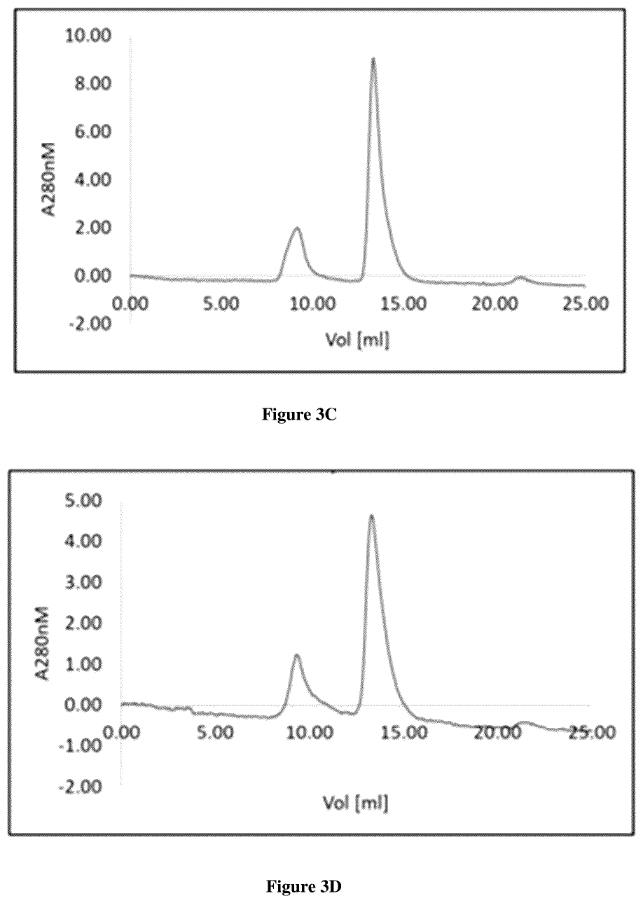

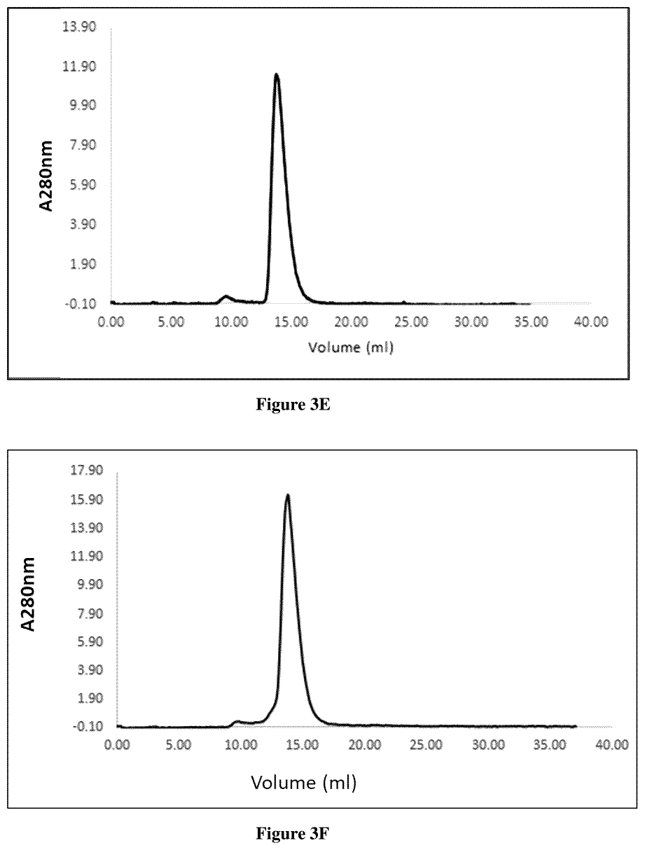

Clones BDG 33.003, BDG 33.004, and BDG 33.005 (Clones C2, C6, and C9, respectively) were expressed and purified as described in Example 1 (Methods), following protein A purification, the IgGs were >95% pure as evident from an SDS PAGE analysis (data not shown). Size exclusion chromatography of BDG 33.003 (clone C2), BDG 33.004 (clone C6), and BDG 33.005 (clone C9) on Superdex®200 10/300, showed two main peaks the first with a retention time of 9.2 ml (0.36 CV), typical of large aggregate and a second peak with retention of approximately 13.2 ml (0.528 CV), typical of an ordinary human IgG1 (hIgG1). The integrated area under the curve of these two peaks showed a ratio of 22% and 78% respectively (FIGS. 3A-3D).

Both BDG33.0023 and BDG33.025 migrated on Superdex®200 10/300 with small leading peak corresponds to (0.36 CV) that is typical of a large diameter aggregate, and a second peak with retention of approximately 13.8 ml (0.55 CV) that is typical of an ordinary human IgG. Area Under the Curve (AUC) peak ratio is 97.3% folded/2.8% misfolded and 98.5% folded/1.5% misfolded for BDG33.023 and BDG33.025 respectively (FIGS. 3E-3F).

To test the thermostability of clones BDG 33.003, BDG 30.004, and BDG 30.005, the clones' thermal melting was monitored by differential scanning fluorescence (DSF) as described in Example 1. As was evident from the first derivative of the fluorescence thermal shift graph, BDG 33.004 had one distinct transition at point at 62° C. which could possibly correspond to both Tm1 and Tm2. (Data not shown). BDG 33.003 and BDG33.005 each had two transition points, a major one at 62° C. (BDG 33.003) and 64.5° C. (BDG 33.005), respectively, and a minor one at 73° C. (BDG 33.003) and 74.5° C. (BDG 33.005), respectively.

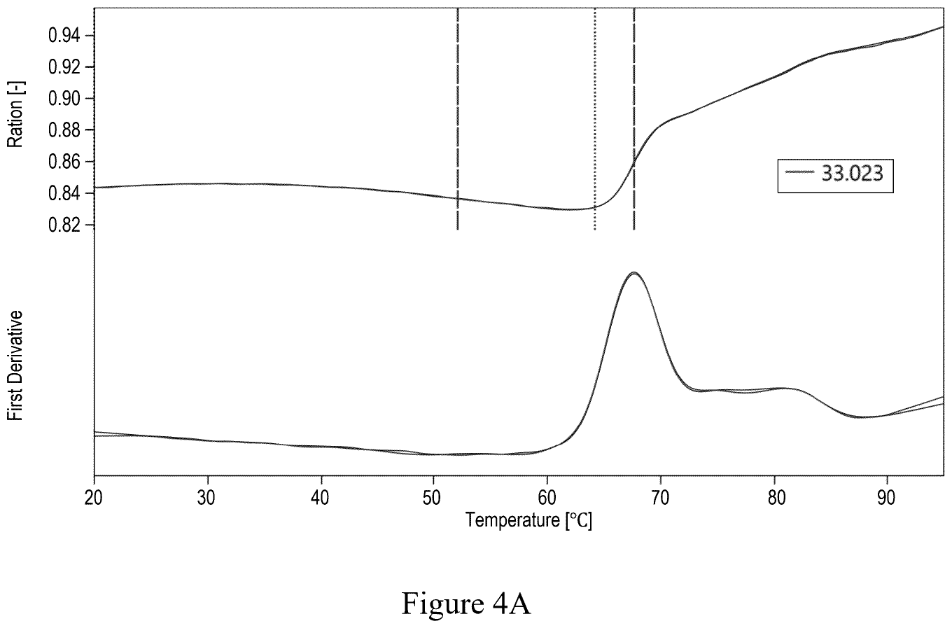

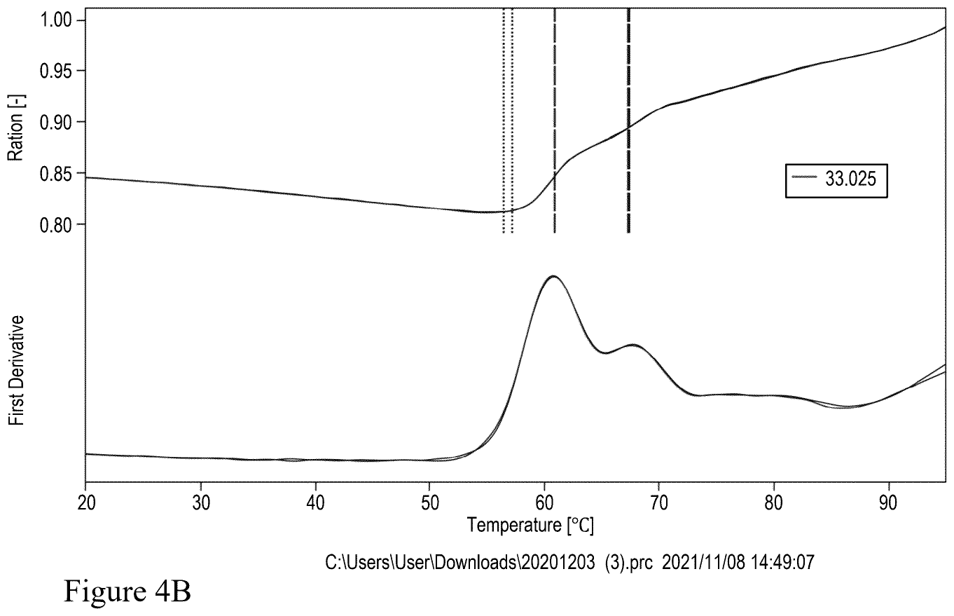

BDG 33.023 and BDG33.025 were tested using NanoDSF Prometheus NT.48 (NanoTemper Technologies, Germany). BDG33.0023 had a T-onset of 64.2° C. and first transition point at 67.7° C., BDG33.0025 had a T-onset of 56.4° C. and first transition point at 60.9° C. and second transition point at 67.4° C. (FIGS. 4A-4B)

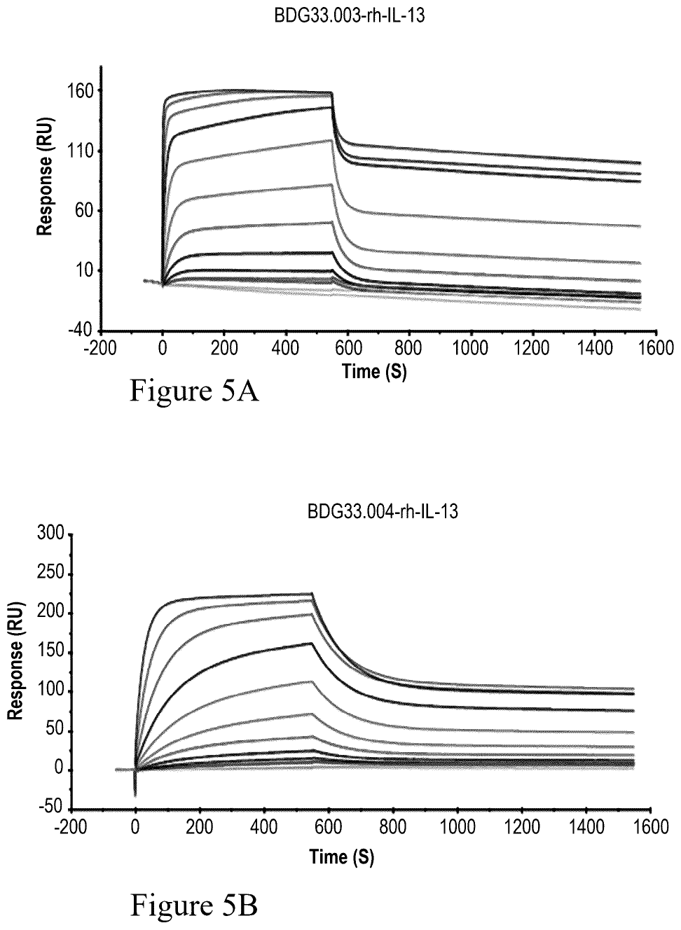

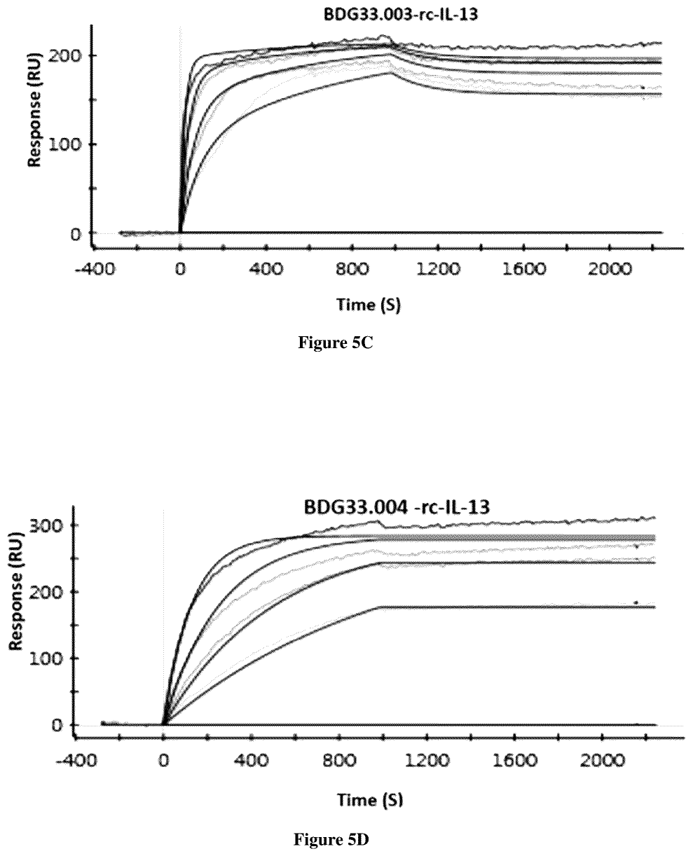

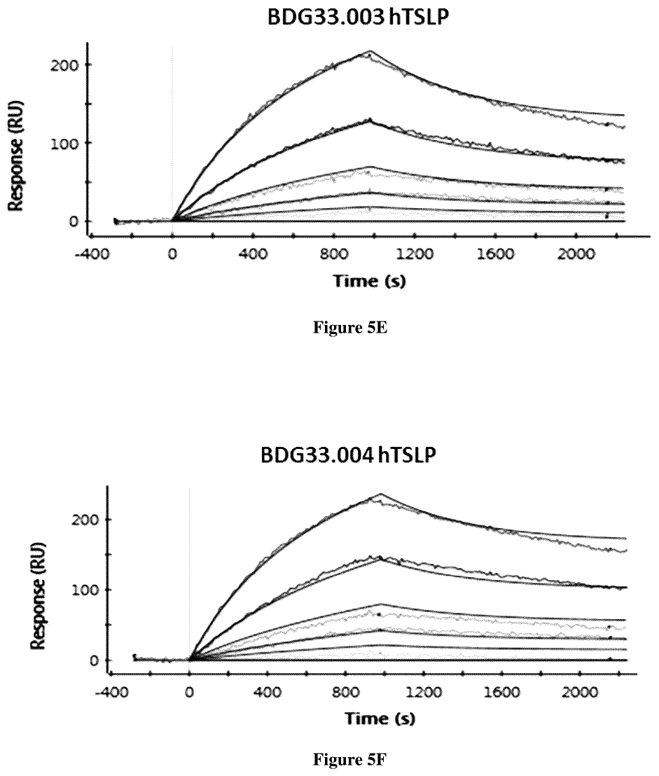

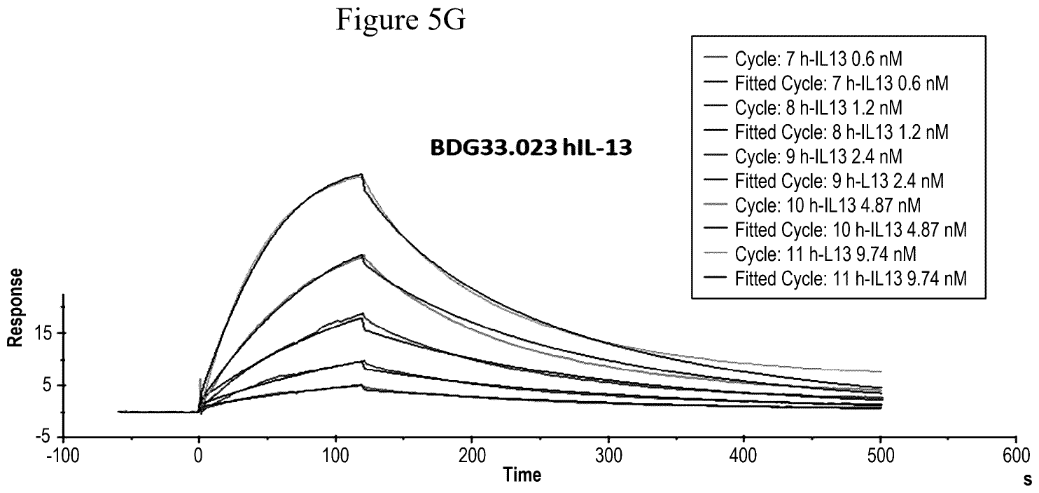

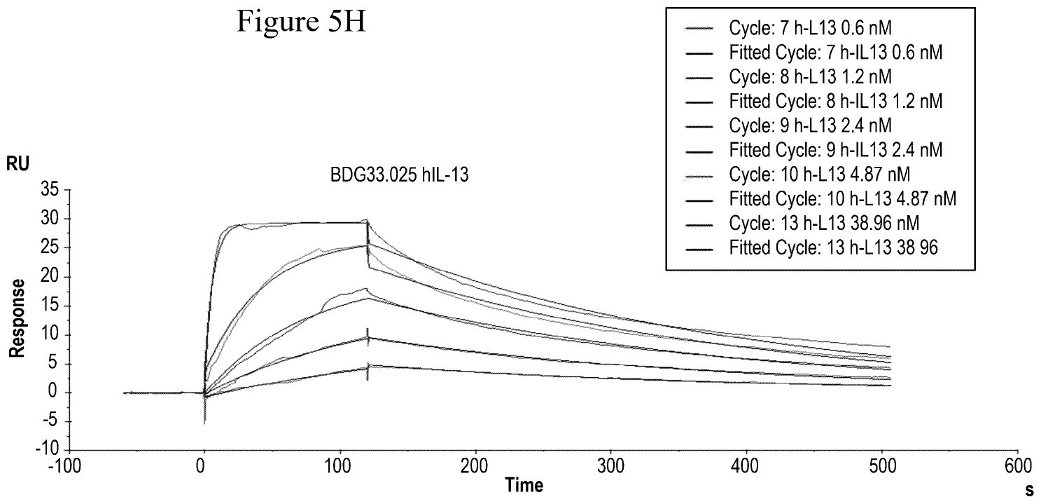

The affinities of the IgGs to human TSLP, human IL-13, and cynomolgus monkey IL-13 were tested. Binding kinetics of hIL-13 to BDG33.023 and BDG33.025 was tested on BJAcoreT200 as described herein, (FIG. 5G-H). BDG33.003 and BDG33.004 clones were tested by SPR analysis on BiacoreT200 and ProteOn™ XPR36, respectively, using the GE capture antibody kit. While it was not possible to obtain kinetics parameters for binding the human IL-13, steady-state binding measurement resulted in an apparent KD of 21.6 nM and 57.4 nM for BDG33.003 and BDG33.004, respectively (FIGS. 5A-5B). For all other measurements of BDG33.003, BDG 33.004, BDG33.023, and BDG33.025, kinetics of binding to hTSLP and hIL-13 are presented in Tables 6A and 6B, and FIGS. 5E and 5H.

The antibodies were also tested for binding of recombinant cynomolgus monkey IL-13 (rc-IL-13), which shares 85% identity and 88% homology with the human IL-13, as can be seen in FIGS. 5C-5D, injection of cIL-13 as analyte, resulted in strong, dose dependent response indicating that BDG33.003 and BDG33.004 bind to rc-IL-13. Although kinetics for rc-IL-13 could not be obtained, the binding and dissociation slopes had similar profile for rh-IL-13 and rc-IL-13, suggesting that the binding mode for recombinant h-IL-13 and rc-IL-13 is likely similar (FIGS. 5A-5B for human IL-13 and FIGS. 5C-5D for cyno IL-13).

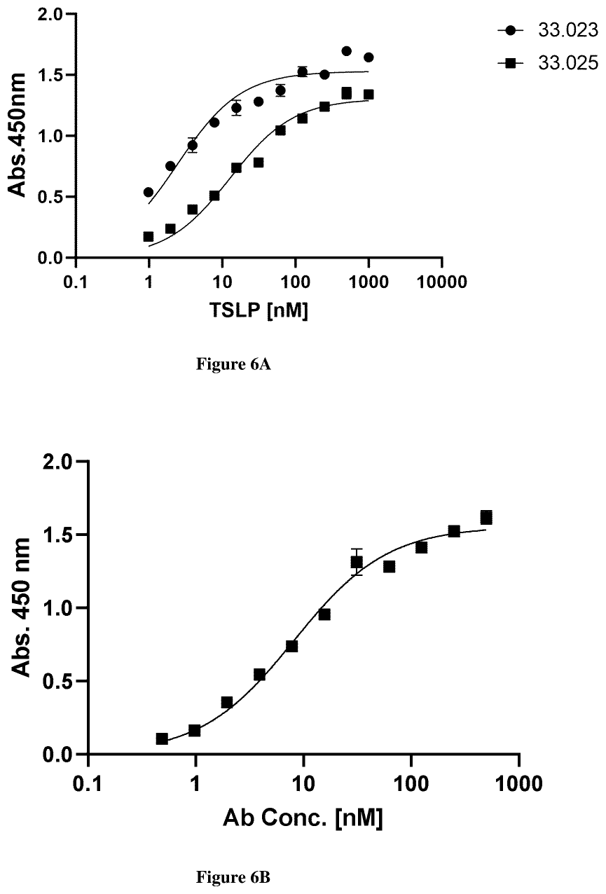

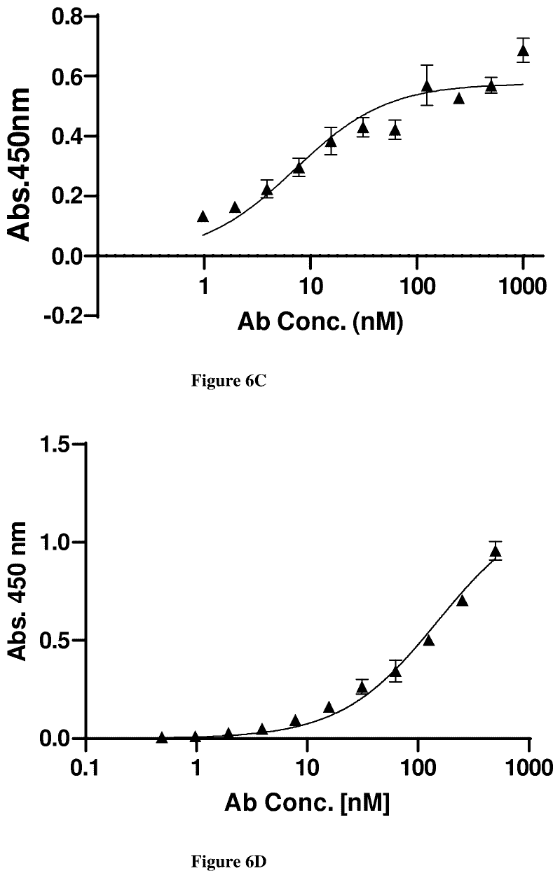

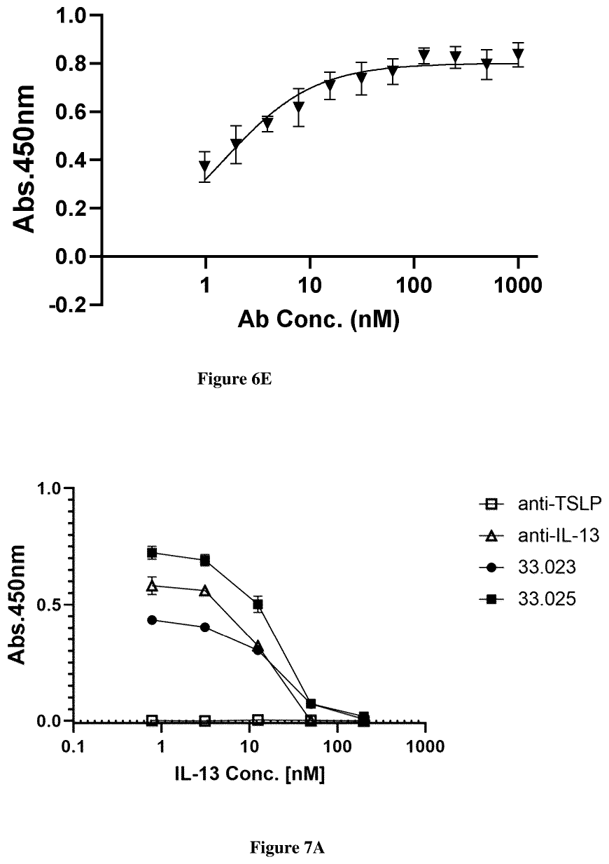

To further test the IgGs affinity to human TSLP, cynomolgus monkey TSLP and cynomolgus monkey IL-13, an ELISA EC50 experiment was done as described herein. Briefly, wells were coated with the respective ligand, then incubated with clones BDG33.003, BDG33.004, BDG33.023, or BDG33.025 at a concentration range of 1 nM to 1000 nM, washed and developed using HRP conjugated secondary antibody. EC50 values are presented in Table 7. Since the IgGs mentioned in the above sections are symmetrical IgGs, and since these same IgGs bind both hIL-13 and hTSLP this data demonstrates that BGD33.003, BGD33.004, BGD33.023, and BGD33.025 antibodies bind the two unrelated targets—TSLP and/or IL-13 from the same standard IgG CDRs, as appose to bi-specific antibody where the Light chain variable domain binds one target and heavy chain binds the other target (FIGS. 6A-6E).

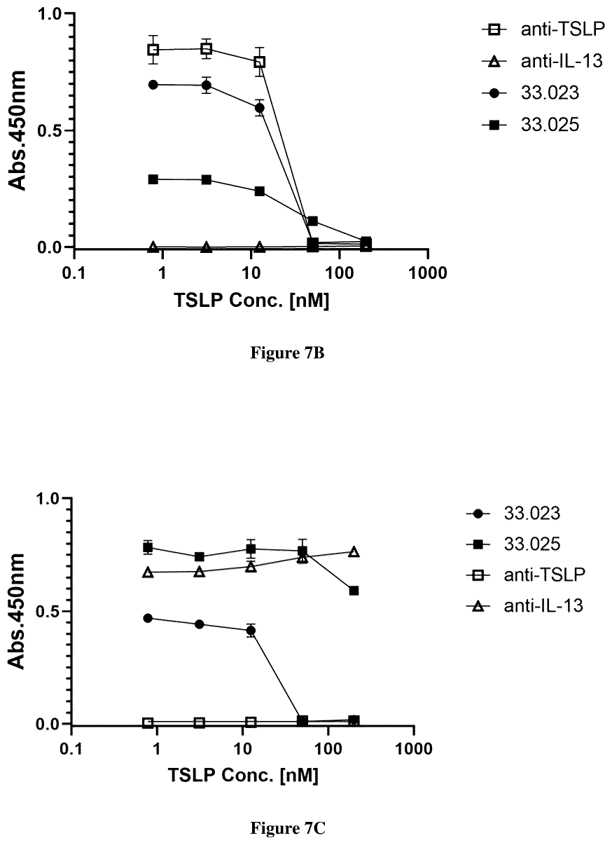

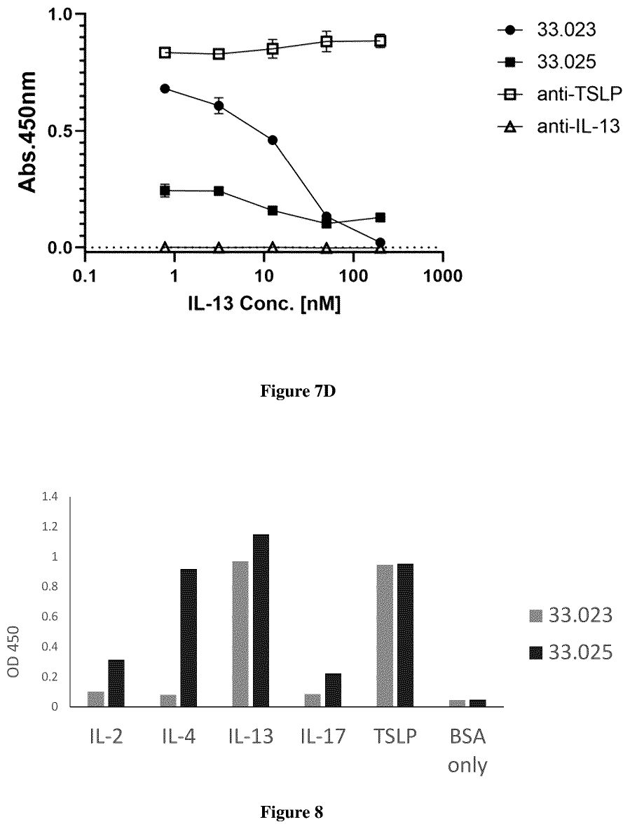

To test whether the IgGs are binding IL-13 and TSLP with overlapping paratopes, a competition assay was done as described herein. Briefly, BDG 33.023 or BDG33.025 were incubated with hIL-13 or hTSLP in concentration range of 0.78 nM to 200 nM and then tested for binding to either IL-13 or TSLP that were pre-coated on an ELISA plate. As can be seen in FIGS. 7A-7D, IL-13 blocks BDG33.023 from binding to IL-13 coated wells, and TSLP blocks BDG33.023 and BDG33.025 from binding to TSLP coated wells (FIGS. 7A-7B). In addition, IL-13 blocked binding of BDG33.023 from binding to TSLP coated wells and reciprocally TSLP blocked binding of BDG33.023 from binding to IL-13 coated wells (FIGS. 7C-7D). This experiment indicates that each of IL-13 and TSLP share at least partially BDG33.023 binding paratope.

IL-13 and TSLP are sequence and structurally unrelated. To test whether binding to these ligands by BDG33.023 and BDG33.025 is specific and not a result of non-specific binding or “stickiness”, BGD33.0023 and BGD33.025 binding to IL-4, IL-2, IL-17, BSA IL-13, and TSLP was tested by ELISA as described herein. As can be seen in FIG. 8, while BDG33.025 shows strong binding to TSLP and IL-4, but not to IL-2 and IL-17. BDG33.023 bind strongly to TSLP and IL-13 but shows no binding to the other ligands, indicating that its binding to IL-13 and TSLP is specific.

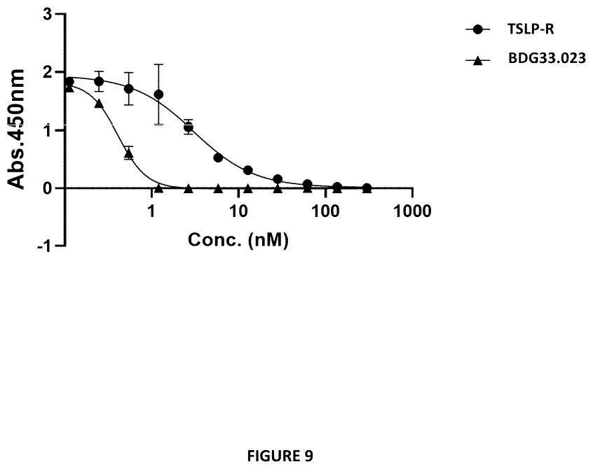

To test if BDG33.023 binds TSLP at a functional epitope, the ability of BDG33.023 to cross-block TSLP from binding to a TSLP receptor was tested. Briefly TSLP-R was coated on ELISA plate wells, and its ability to bind hTSLP in the presence of 0 nM to 500 nM BDG33.023 was tested. As can be seen in FIG. 9, BDG 33.023 can cross block TSLP binding to TSLP-R with an IC 50 of 0.41 nM indicating that BDG33.023 binds tightly TSLP at a biologically functional site.

TABLE 6A
KD values of antibody clones for human IL-13 and TSLP

Human IL-13

| Antibody | K_{D (Steady state) (M)} | SE(K_{D}) (M) |
|---|---|---|
| BDG33.003 | 2.16E−08 | 2.5E−09 |
| BDG33.004 | 5.74E−08 | 4.1E−09 |

| | Human TSLP | | |
|---|---|---|---|
| | ka (1/Ms) | kd (1/s) | KD (M) |
| BDG33.003 | 2.87E+05 | 1.29E−3 | 4.5E−9 |
| BDG33.004 | 3.44E+05 | 1.14E−3 | 3.33E−9 |

TABLE 6B
KD values of antibody clones for human IL-13 and TSLP

| | Human IL-13 | | |
|---|---|---|---|
| Antibody | ka (1/Ms) | kd (1/s) | KD (M) |
| BDG33.023 | 4.31E+06 | 0.01573 | 3.65E−09 |
| BDG33.025 | 4.07E+6 | 0.0036 | 9.03E−10 |

TABLE 7
EC50 values for human and cyno TSLP, and cyno IL-13

| | EC50 value for ligand | | |
|---|---|---|---|
| IgG | hTSLP | cTSLP | cIL-13 |
| BDG33.023 | 2.5 nM | 8.2 nM | 7.1 nM |
| BDG33.025 | 12.8 nM | 148 nM | 1.5 nM |

### Example 4: Cell Based Assays for the Inhibitor Antibody BDG33.003 (Clone C2)

Objective: Analyze the IgG1 antibodies for the ability to inhibit IL-13 activity.

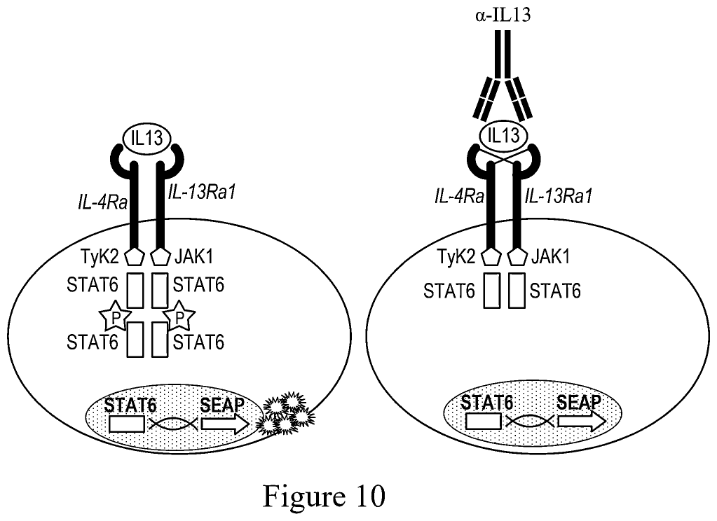

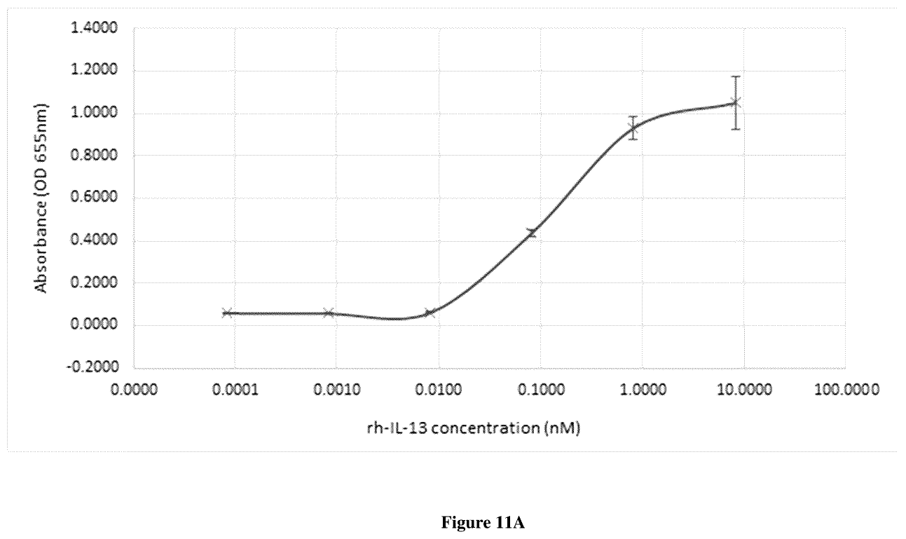

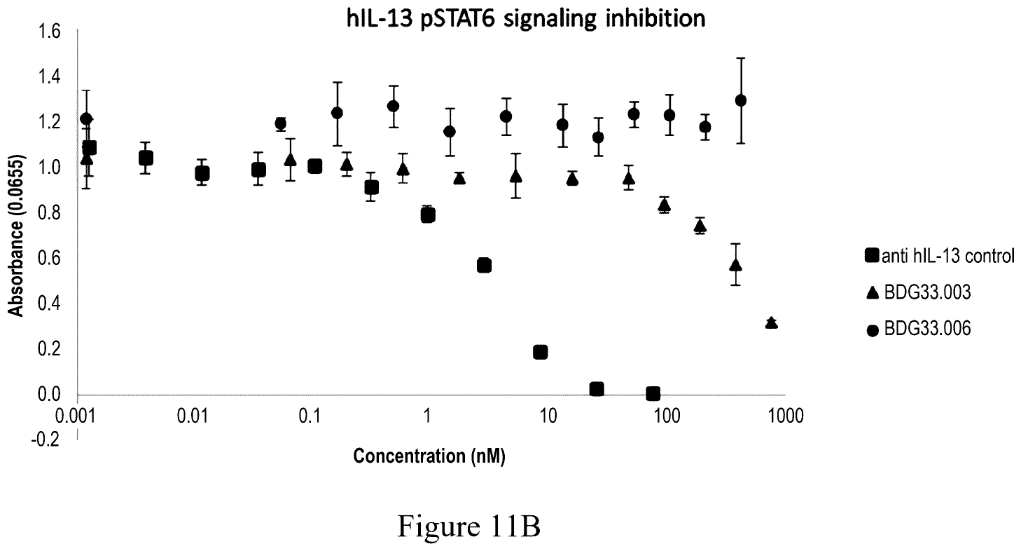

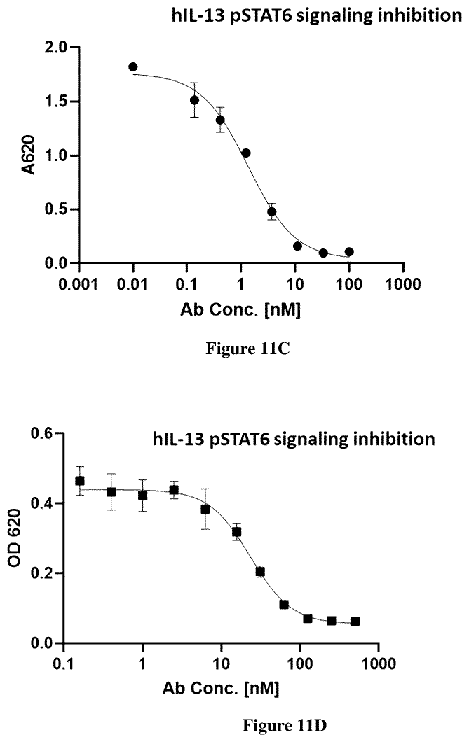

Results: To evaluate the capability of the antibody to inhibit rh-IL-13, the HEK-Blue IL-4/IL-13 system was used. The system uses HEK293 cells, which were stably transfected with human STAT6 gene and the reporter gene secreted embryonic alkaline phosphatase (SEAP) under the control of the IFNβ minimal promoter fused to four STAT6 binding sites (Example 1 (Methods), and FIG. 10). The system was initially tested by introducing rh-IL-13 to the cells and following the cell signaling cascade resulting in IL-13R (IL-13 receptor) activation by rh-IL-13. The results showed that IL-13 had an EC50 of about 0.12 nM to the cells (FIG. 11). Next, the engineered BDG33.003 (clone C2), BDG33.023 and BDG33.025 antibodies were tested to determine if they could inhibit IL-13 mediated activation of the cell's signaling cascade. The antibody was incubated with 0.4 nM rh-IL-13, which was shown to activate IL-13R to approximately 70% of the saturation level, and the IgG/IL-13 mixture was introduced to the cells for 24 hrs. The results obtained showed that the antibodies were able to inhibit IL-13 from binding to the IL-13R/IL-4R receptor complex, thus interfering with the signaling cascade. While for BDG33.003 the exact IC50 value was hard to determine, it is clear that BDG33.003 is inhibiting IL-13 signaling cascade. In addition, BDG33.023 and BDG33.025 inhibited IL-13 signaling cascade with an IC50 of 1.3 nM and 25 nM respectively, indicating that the IgGs are functionally blocking IL-13 in a biologically relevant setting. (FIGS. 11B-11D).

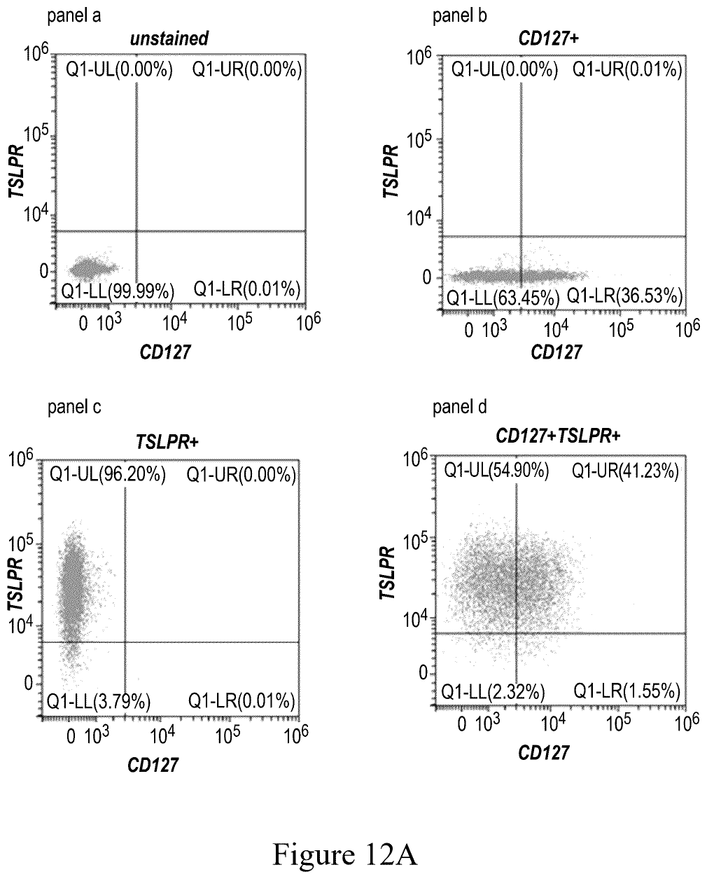

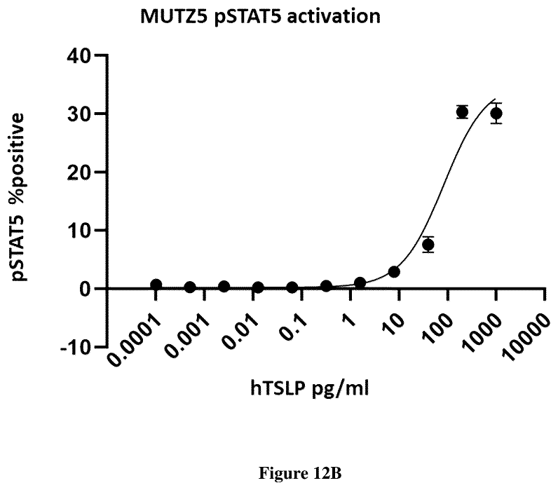

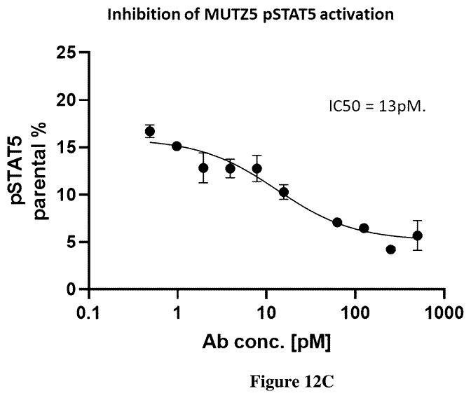

To evaluate the capability of the antibodies to inhibit human TSLP in cells, MUTZ5 cells were used to test pSTAT5 TSLP dependent activation in a similar manner reported by Francis O L et al (Hematopoiesis 2016). TSLP induced phospho-STATS (pSTAT5) cellular activation cascade requires IL-7 receptor and TSLP-R receptor to function, as can be seen in FIG. 12A both are expressed on the MUTZ5 cell line surface indicating that these cells have the necessary receptors for this assay. To establish the cellular response to TSLP cells were incubated with TSLP at a concentration range of 0.0001 to 1000 pg/ml and their pSTAT5 activation levels were determined by flow cytometry. As can be seen in FIG. 12B treatment of MUTZ 5 cells with TSLP activates pSTAT5 in a dose dependent manner, indicating that these cells respond to TSLP via the pSTAT5 pathway. To test BDG33.023 inhibition of TSLP dependent STATS activation, TSLP at a concentration of 14 pM was mixed with 0.48 pM to 500 pM BDG33.023 and incubated with MUTZ cells, as can be seen in FIG. 12C. BDG33.023 inhibits TSLP pSTAT5 activation with an IC50 value of 13 pM. These experiments demonstrate that BDG33.023 is functionally blocking TSLP in a biologically relevant cell-based setting.

Summary: The “re-epitoped” engineered BDG33.003 (clone 2), BDG33.023, and BDG33.025 antibodies were shown to bind both TSLP and IL-13 In contrast to the bispecific antibody format where each Fv has a specificity to a single antigen, these three antibodies are a standard IgG format, and each Fv has specificity to both IL-13 and TSLP In addition BDG33.023's paratopes for IL-13 and TSLP was shown to be at least partly overlapping. All three IgGs interfere with the IL-13R/IL4R and TSLPR/IL-7R signaling cascade. Such antibodies could be used as a component of a therapeutic treatment, for example but not limited to severe asthma, atopic dermatitis, and other allergic and respiratory conditions.

### Example 5: Biochemical Characterization of Dual Binding Antibodies

Objective: To examine the biochemical and functional properties of dual binding antibodies BDG38.074 to BDG38.143.

Methods:

Peripheral blood mononuclear cells (PBMCs) (Cell Generation, CAT: 101061021) were used to determine IL-13 and hTLSP inhibition. PBMCs were thawed and cultured in growth medium comprising of RPMI—1640, 10% PBS, 1% Glutamax, 1% Sodium-Pyruvate, 0.1% 2-ME, 1% Pen-Strep and 1% nonessential AA. The cells were seeded in 96 well plate at 5×10⁵ cells/well. Fifteen ng/mL hTSLP and 1.25 ng/mL IL-13 were incubated with antibodies for half an hour in 37° C., then added to the cells, to a total of 200 uL. Cells were incubated for 48 hours at 37° C., 5% CO₂.

CD23 upregulates in human monocytes in the presence of IL-13 (RD May et. al, 2011). IC₅₀ of antibody inhibition of IL-13 was determined by measuring CD23 expression level in monocytes. At the end of 48 hours incubation of the cells with different concentrations of antibodies, monocytes were detached from the bottom of the wells using cold PBS and scraping. Cells were marked using CD3 (Bio Legend, CAT: 300450), CD14 (Bio Legend, CAT: 301814), CD19 (Bio Legend, CAT: 302212) and CD23 (Bio Legend, CAT: 338506) antibodies. CD23 percentage of CD14+ population was measured using CytoFLEX flow cytometer (Beckman Coulter).

IC₅₀ of antibody inhibition of hTSLP was determined by TARC inhibition. TARC levels were determined using TARC DUOSET ELISA kit DY364 (R&D systems) according to kit instructions. Briefly, ELISA high bonding protein plates were plated with capture (non-biotinylated) antibody, diluted in PBSX1. Plates were sealed and incubated overnight. The following day plates were washed and blocked using PBST 2% BSA in room temperature, shaking, for two hours. Supernatant from the PBMCs plates was transferred to the wells. Detection was preformed using the kit's detection antibodies (Biotinylated) in PBS 1% BSA and Streptavidin-HRP in PBST 2% BSA. After adding TMB stop solution, ELISA plates were read at 450 nm. Values were analyzed using standard sample curve.

Other methods used to provide the results described and presented in this Example have been described in Example 1 above.

Results: The tables below present the amino acid sequences for antibodies BDG38.074 to BDG38.143. The VH and VL sequences are shown in Table 10, whereas the heavy and light chain CDRs are shown in Table 8 and 9 respectively.

TABLE 8
Amino Acid Sequences of Heavy-Chain
CDR Regions for Antibodies BDG38.074
to BDG38.143

| | | SEQ | | SEQ | | SEQ |
|---|---|---|---|---|---|---|
| | | ID | | ID | | ID |
| Antibodies | HCDR1 | NO | HCDR2 | NO | HCDR3 | NO |
| BDG38_074_VH | GFAFRTYG | 349 | IWDDGSNT | 350 | VRAPQWYLTAEAFDL | 351 |
| BDG38_079_VH | GFAFRTYG | 349 | IWYDGSNT | 356 | VRAPQWYLTAEAFDL | 351 |
| BDG38_094_VH | GFAFRTYG | 349 | IWDDGSNT | 350 | VRAPQWYLTAEAFDL | 351 |
| BDG38_138_VH | GFAFRTYG | 349 | IWDDGSNT | 350 | VRAPQWYLTAEAFDL | 351 |
| BDG38_075_VH | GFAFRTYG | 349 | IWYDGSAT | 352 | VRAPQWYLSAEAFDL | 353 |
| BDG38_076_VH | GFAFRTYG | 349 | IWDDGSAT | 354 | VRAPQWYLTAEAFDL | 351 |
| BDG38_077_VH | GFAFRTYG | 349 | IWYDGSAT | 352 | VRAPQWYLTAEAFDL | 351 |
| BDG38_078_VH | GFEFRTYG | 355 | IWYDGSNT | 356 | VRAPQWYLTAEAFDL | 351 |
| BDG38_080_VH | GFAFRTYG | 349 | IWYDGSAT | 352 | VRAPQWYLSAEAFDL | 353 |
| BDG38_081_VH | GFAFRTYG | 349 | IWDDGSNT | 350 | VRAPQWYLTAEAFDL | 351 |
| BDG38_082_VH | GFAFRTYG | 349 | IWDDGSNT | 350 | VRAPQWYLTAEAFDL | 351 |
| BDG38_083_VH | GFEFRTYG | 355 | IWDDGSNT | 350 | ARAPQWYLTAEAFDL | 357 |
| BDG38_084_VH | GFAFRTYG | 349 | IWDDGSNT | 350 | VRAPQWYLTAEAFDL | 351 |
| BDG38_085_VH | GFEFRTYG | 355 | IWYDGSNT | 356 | VRAPQWYLTAEAFDL | 351 |
| BDG38_086_VH | GFEFRTYG | 355 | IWYDGSNT | 356 | VRAPQWYLTAEAFDL | 351 |
| BDG38_087_VH | GFAFRTYG | 349 | IWDDGSNT | 350 | VRAPQWYLTAEAFDL | 351 |
| BDG38_088_VH | GFEFRTYG | 355 | IWYDGSNT | 356 | VRAPQWYLTAEAFDL | 351 |
| BDG38_089_VH | GFEFRTYG | 355 | IWYDGSAT | 352 | VRAPQWYLTAEAFDL | 351 |
| BDG38_090_VH | GFEFRTYG | 355 | IWDDGSNT | 350 | ARAPQWYLTAEAFDL | 357 |
| BDG38_091_VH | GFEFRTYG | 355 | IWYDGSNT | 356 | VRAPQWYLTAEAFDL | 351 |
| BDG38_092_VH | GFEFRTYG | 355 | IWDDGSNT | 350 | VRAPQWYLTAEAFDL | 351 |
| BDG38_093_VH | GFEFRTYG | 355 | IWYDGSNT | 356 | VRAPQWYLTAEAFDL | 351 |
| BDG38_095_VH | GFEFRTYG | 355 | IWYDGSAT | 352 | VRAPQWYLTAEAFDL | 351 |
| BDG38_096_VH | GFEFRTYG | 355 | IWDDGSNT | 350 | VRAPQWYLTAEAFDL | 351 |
| BDG38_097_VH | GFEFRTYG | 355 | IWYDGSAT | 352 | VRAPQWYLTAEAFDL | 351 |
| BDG38_098_VH | GFEFRTYG | 355 | IWDDGSNT | 350 | VRAPQWYLTAEAFDL | 351 |
| BDG38_099_VH | GFEFRTYG | 355 | IWYDGSNT | 356 | VRAPQWYLTAEAFDL | 351 |
| BDG38_100_VH | GFEFRTYG | 355 | IWYDGSNT | 356 | VRAPQWYLTAEAFDL | 351 |
| BDG38_101_VH | GFEFRTYG | 355 | IWYDGSAT | 352 | VRAPQWYLTAEAFDL | 351 |
| BDG38_102_VH | GFEFRTYG | 355 | IWYDGSNT | 356 | VRAPQWYLTAEAFDL | 351 |
| BDG38_103_VH | GFEFRTYG | 355 | IWYDGSNT | 356 | VRAPQWYLTAEAFDL | 351 |
| BDG38_104_VH | GFEFRTYG | 355 | IWYDGSAT | 352 | VRAPQWYLTAEAFDL | 351 |
| BDG38_105_VH | GFEFRTYG | 355 | IWYDGSNT | 356 | VRAPQWYLTAEAFDL | 351 |
| BDG38_106_VH | GFEFRTYG | 355 | IWDDGSNT | 350 | VRAPQWYLTAEAFDL | 351 |
| BDG38_107_VH | GFEFRTYG | 355 | IWYDGSNT | 356 | VRAPQWYLTAEAFDL | 351 |
| BDG38_108_VH | GFEFRTYG | 355 | IWYDGSNT | 356 | VRAPQWYLTAEAFDL | 351 |
| BDG38_109_VH | GFEFRTYG | 355 | IWYDGSNT | 356 | VRAPQWYLTAEAFDL | 351 |
| BDG38_110_VH | GFEFRTYG | 355 | IWDDGSNT | 350 | VRAPQWYLTAEAFDL | 351 |
| BDG38_111_VH | GFEFRTYG | 355 | IWDDGSAT | 354 | VRAPQWYLTAEAFDL | 351 |
| BDG38_112_VH | GFEFRTYG | 355 | IWYDGSNT | 356 | VRAPQWYLTAEAFDL | 351 |
| BDG38_113_VH | GFEFRTYG | 355 | IWYDGSNT | 356 | VRAPQWYLTAEAFDL | 351 |
| BDG38_114_VH | GFEFRTYG | 355 | IWYDGSNT | 356 | VRAPQWYLTAEAFDL | 351 |
| BDG38_115_VH | GFEFRTYG | 355 | IWYDGSNT | 356 | VRAPQWYLTAEAFDL | 351 |
| BDG38_116_VH | GFEFRTYG | 355 | IWDDGSNT | 350 | VRAPQWYLTAEAFDL | 351 |
| BDG38_117_VH | GFEFRTYG | 355 | IWDDGSNT | 350 | ARAPQWYLTAEAFDL | 357 |
| BDG38_118_VH | GFEFRTYG | 355 | IWYDGSAT | 352 | VRAPQWYLTAEAFDL | 351 |
| BDG38_119_VH | GFEFRTYG | 355 | IWYDGSNT | 356 | VRAPQWYLTAEAFDL | 351 |
| BDG38_120_VH | GFEFRTYG | 355 | IWDDGSNT | 350 | VRAPQWYLTAEAFDL | 351 |
| BDG38_121_VH | GFEFRTYG | 355 | IWYDGSNT | 356 | VRAPQWYLTAEAFDL | 351 |
| BDG38_122_VH | GFEFRTYG | 355 | IWDDGSNT | 350 | VRAPQWYLTAEAFDL | 351 |
| BDG38_123_VH | GFEFRTYG | 355 | IWDDGSNT | 350 | VRAPQWYLTAEAFDL | 351 |
| BDG38_124_VH | GFEFRTYG | 355 | IWDDGSNT | 350 | VRAPQWYLTAEAFDL | 351 |
| BDG38_125_VH | GFEFRTYG | 355 | IWDDGSNT | 350 | VRAPQWYLTAEAFDL | 351 |
| BDG38_126_VH | GFEFRTYG | 355 | IWDDGSNT | 350 | VRAPQWYLTAEAFDL | 351 |
| BDG38_127_VH | GFEFRTYG | 355 | IWYDGSAT | 352 | ARAPQWYLTAEAFDL | 357 |
| BDG38_128_VH | GFEFRTYG | 355 | IWDDGSNT | 350 | VRAPQWYLTAEAFDL | 351 |
| BDG38_129_VH | GFEFRTYG | 355 | IWDDGSNT | 350 | ARAPQWYLTAEAFDL | 357 |
| BDG38_130_VH | GFAFRTYG | 349 | IWDDGSNT | 350 | VRAPQWYLTAEAFDL | 351 |
| BDG38_131_VH | GFEFRTYG | 355 | IWDDGSNT | 350 | VRAPQWYLTAEAFDL | 351 |
| BDG38_132_VH | GFEFRTYG | 355 | IWDDGSNT | 350 | VRAPQWYLTAEAFDL | 351 |
| BDG38_133_VH | GFEFRTYG | 355 | IWDDGSNT | 350 | VRAPQWYLTAEAFDL | 351 |
| BDG38_134_VH | GFEFRTYG | 355 | IWYDGSNT | 356 | VRAPQWYNSAEAFDL | 358 |
| BDG38_135_VH | GFAFRTYG | 349 | IWYDGSNT | 356 | VRAPQWYNSAEAFDL | 358 |
| BDG38_136_VH | GFEFRTYG | 355 | IWYDGSNT | 356 | VRAPQWYNSAEAFDL | 358 |
| BDG38_137_VH | GFEFRTYG | 355 | IWDDGSNT | 350 | VRAPQWYLTAEAFDL | 351 |
| BDG38_139_VH | GFAFRTYG | 349 | IWDDGSNT | 350 | VRAPQWYLSAEAFDL | 353 |
| BDG38_140_VH | GFEFRTYG | 355 | IWDDGSNT | 350 | ARAPQWYLTAEAFDL | 357 |
| BDG38_141_VH | GFEFRTYG | 355 | IWDDGSNT | 350 | VRAPQWYLTAEAFDL | 351 |
| BDG38_142_VH | GFEFRTYG | 355 | IWDDGSNT | 350 | VRAPQWYLTAEAFDL | 351 |
| BDG38_143_VH | GFEFRTYG | 355 | IWDDGSAT | 354 | VRAPQWYLTAEAFDL | 351 |

TABLE 9
Amino Acid Sequences of Light-Chain
CDR Regions for Antibodies BDG38.074 to
BDG38.143

| | | SEQ | | | SEQ |
|---|---|---|---|---|---|
| | | ID | | | ID |
| Antibodies | LCDR1 | NO | LCDR2 | LCDR3 | NO |
| BDG38_074_VL | MIGAYL | 359 | DDV | QVWDHNTDKMV | 361 |
| BDG38_079_VL | MIGGYL | 364 | DDV | QVWDHDSNTMV | 371 |
| BDG38_094_VL | LIGAYL | 362 | DDV | QVWDHNSNHMV | 384 |
| BDG38_138_VL | MIGGYL | 364 | DDV | QVWDHNSNHMV | 384 |
| BDG38_075_VL | LIGAYL | 362 | DDV | QVWDHDTNTMV | 363 |
| BDG38_076_VL | MIGGYL | 364 | DDV | QVWDHDTNHMV | 365 |
| BDG38_077_VL | LIGSRL | 366 | DDS | QVWDHSSNTYV | 368 |
| BDG38_078_VL | LIGARL | 369 | DDS | QVWDYYSDHMV | 370 |
| BDG38_080_VL | LIGAYL | 362 | DDS | QVWDHNTNHMV | 372 |
| BDG38_081_VL | MIGAYL | 359 | DDV | QVWDHNTQQMV | 373 |
| BDG38_082_VL | MIGGYL | 364 | DDV | QVWDHDTNQVV | 374 |
| BDG38_083_VL | LIGAKL | 375 | DDS | QVWDYSSDTMV | 376 |
| BDG38_084_VL | LIGAYL | 362 | DDV | QVWDHSTNTMV | 377 |
| BDG38_085_VL | LIGARL | 369 | DDS | QVWDYSSNSYV | 378 |
| BDG38_086_VL | LIGARL | 369 | DDS | QVWDYSSNTYV | 379 |
| BDG38_087_VL | MIGAYL | 359 | DDV | QVWDHNSNQMV | 380 |
| BDG38_088_VL | LIGARL | 369 | DDS | QVWDYSSNTYV | 379 |
| BDG38_089_VL | LIGARL | 369 | DDS | QVWDHSSNHYV | 381 |
| BDG38_090_VL | LIGARL | 369 | DDS | QVWDYYSDHMV | 370 |
| BDG38_091_VL | LIGSRL | 366 | DDS | QVWDHYSNHYV | 382 |
| BDG38_092_VL | LIGARL | 369 | DDS | QVWDYYSDHMV | 370 |
| BDG38_093_VL | LIGARL | 369 | DDS | QVWDYSADSYV | 383 |
| BDG38_095_VL | LIGSRL | 366 | DDS | QVWDYYSDSYV | 385 |
| BDG38_096_VL | LIGARL | 369 | DDS | QVWDYSSDSMV | 386 |
| BDG38_097_VL | LIGARL | 369 | DDS | QVWDYYSDHYV | 387 |
| BDG38_098_VL | LIGARL | 369 | DDS | QVWDYSSDSMV | 386 |
| BDG38_099_VL | LIGARL | 369 | DDS | QVWDYSSDSYV | 388 |
| BDG38_100_VL | LIGARL | 369 | DDS | QVWDYYSNSYV | 389 |
| BDG38_101_VL | LIGARL | 369 | DDS | QVWDYSSNTYV | 379 |
| BDG38_102_VL | LIGARL | 369 | DDS | QVWDYSSNTYV | 379 |
| BDG38_103_VL | LIGARL | 369 | DDS | QVWDYYSNSYV | 389 |
| BDG38_104_VL | LIGARL | 369 | DDS | QVWDYYSDSYV | 390 |
| BDG38_105_VL | LIGARL | 369 | DDS | QVWDYYSNSYV | 389 |
| BDG38_106_VL | LIGSRL | 366 | DDS | QVWDHYSDHMV | 391 |
| BDG38_107_VL | LIGARL | 369 | DDS | QVWDYSSDSYV | 388 |
| BDG38_108_VL | LIGARL | 369 | DDS | QVWDYYSNSYV | 389 |
| BDG38_109_VL | LIGARL | 369 | DDS | QVWDYSSNSYV | 378 |
| BDG38_110_VL | LIGAKL | 375 | DDS | QVWDYSSNHMV | 392 |
| BDG38_111_VL | LIGARL | 369 | DDS | QVWDYYANSYV | 393 |
| BDG38_112_VL | LIGARL | 369 | DDS | QVWDYSSDTYV | 394 |
| BDG38_113_VL | LIGARL | 369 | DDS | QVWDYSSDSYV | 388 |
| BDG38_114_VL | LIGARL | 369 | DDS | QVWDYSANSYV | 395 |
| BDG38_115_VL | LIGARL | 369 | DDS | QVWDYSSNTYV | 379 |
| BDG38_116_VL | LIGARL | 369 | DDS | QVWDYYSDTMV | 396 |
| BDG38_117_VL | LIGAKL | 375 | DDS | QVWDYSSDTMV | 376 |
| BDG38_118_VL | LIGARL | 369 | DDS | QVWDYSSDHYV | 397 |
| BDG38_119_VL | LIGARL | 369 | DDS | QVWDYSSNTYV | 379 |
| BDG38_120_VL | LIGARL | 369 | DDS | QVWDYSSDHMV | 398 |
| BDG38_121_VL | LIGARL | 369 | DDS | QVWDYSSNTYV | 379 |
| BDG38_122_VL | LIGARL | 369 | DDS | QVWDYSSDSYV | 388 |
| BDG38_123_VL | LIGARL | 369 | DDS | QVWDYSSDTYV | 394 |
| BDG38_124_VL | LIGARL | 369 | DDS | QVWDYSSDTYV | 394 |
| BDG38_125_VL | LIGARL | 369 | DDS | QVWDYSSDHMV | 398 |
| BDG38_126_VL | LIGAKL | 375 | DDS | QVWDYSSDHMV | 398 |
| BDG38_127_VL | LIGAKL | 375 | DDS | QVWDYYSDTYV | 399 |
| BDG38_128_VL | LIGAKL | 375 | DDS | QVWDYYADTMV | 400 |
| BDG38_129_VL | LIGAKL | 375 | DDS | QVWDYSSDHMV | 398 |
| BDG38_130_VL | LIGARL | 369 | DDS | QVWDYSSNSYV | 378 |
| BDG38_131_VL | LIGAKL | 375 | DDS | QVWDYYSNTMV | 401 |
| BDG38_132_VL | LIGARL | 369 | DDS | QVWDYSSDHMV | 398 |
| BDG38_133_VL | LIGARL | 369 | DDS | QVWDYSSDTYV | 394 |
| BDG38_134_VL | LIGARL | 369 | DDS | QVWDYSADTMV | 402 |
| BDG38_135_VL | LIGARL | 369 | DDS | QVWDHSADTMV | 403 |
| BDG38_136_VL | LIGAKL | 375 | DDS | QVWDYSSDTMV | 376 |
| BDG38_137_VL | LIGARL | 369 | DDS | QVWDYSADTMV | 402 |
| BDG38_139_VL | MIGAYL | 359 | DDV | QVWDHNSDHMV | 404 |
| BDG38_140_VL | LIGAKL | 375 | DDS | QVWDYSANHMV | 405 |
| BDG38_141_VL | LIGSRL | 366 | DDS | QVWDYYSNHMV | 406 |
| BDG38_142_VL | LIGAKL | 375 | DDS | QVWDYYSHTMV | 407 |
| BDG38_143_VL | LIGARL | 369 | DDS | QVWDYSSNTYV | 379 |

TABLE 10
Amino Acid Sequences of VH and VL
Regions for Antibodies BDG38.074 to
BDG38.143

| | | SEQ ID |
|---|---|---|
| Antibodies | Amino Acid Sequence | NO: |
| BDG38_074_ | QMQLVESGGGVVQPGRSLRLSCAASGFAFRTYGMHWVRQAPGKG | 209 |
| VH | LEWVAVIWDDGSNTHYADSVKGRFTITRDNSKNTLNLQMNSLRAE | |
| | DTAVYYCVRAPQWYLTAEAFDLWGQGTMVTVSS | |
| BDG38_074_ | SYVLTQPPSVSVAPGQTATITCGGNMIGAYLVHWYQQKPGQAPLL | 210 |
| VL | VVYDDVDRPNRIPERFSGSNSGNTATLTISRVEAGDEADYYCQVWD | |
| | HNTDKMVFGGGTKLTVL | |
| BDG38_079_ | QMQLVESGGGVVQPGRSLRLSCAASGFAFRTYGMHWVRQAPGKG | 219 |
| VH | LEWVAVIWYDGSNTHYADSVKGRFTITRDNSKNTLNLQMNSLRAE | |
| | DTAVYYCVRAPQWYLTAEAFDLWGQGTMVTVSS | |
| BDG38_079_ | SYVLTQPPSVSVAPGETATITCGGNMIGGYLVHWYQQKPGQAPLLV | 220 |
| VL | IYDDVDRPDRIPERFSGSNSGNTATLTISRVEAGDEADYYCQVWDH | |
| | DSNTMVFGGGTKLTVL | |
| BDG38_094_ | QMQLVESGGGVVQPGRSLRLSCAASGFAFRTYGMHWVRQAPGKG | 249 |
| VH | LEWVAVIWDDGSNTHYADSVKGRFTITRDNSKNTLNLQMNSLRAE | |
| | DTAVYYCVRAPQWYLTAEAFDLWGQGTMVTVSS | |
| BDG38_094_ | SYVLTQPPSVSVAPGETASITCGGNLIGAYLVHWYQQKPGQAPLLVI | 250 |
| VL | YDDVDRPARIPERFSGSNSGNTATLTISRVEAGDEADYYCQVWDHN | |
| | SNHMVFGGGTKLTVL | |
| BDG38_138_ | QMQLVESGGGVVQPGRSLRLSCAASGFAFRTYGMHWVRQAPGKG | 337 |
| VH | LEWVAVIWDDGSNTHYADSVKGRFTITRDNSKNTLNLQMNSLRAE | |
| | DTAVYYCVRAPQWYLTAEAFDLWGQGTMVTVSS | |
| BDG38_138_ | SYVLTQPPSVSVAPGETASITCGGNMIGGYLVHWYQQKPGQAPVLV | 338 |
| VL | IYDDVDRPSRIPERFSGSNSGNTATLTISRVEAGDEADYYCQVWDH | |
| | NSNHMVFGGGTKLTVL | |
| BDG38_075_ | QMQLVESGGGVVQPGRSLRLSCAASGFAFRTYGMHWVRQAPGKG | 211 |
| VH | LEWVAVIWYDGSATHYADSVKGRFTITRDNSKNTLNLQMNSLRAE | |
| | DTAVYYCVRAPQWYLSAEAFDLWGQGTMVTVSS | |
| BDG38_075_ | SYVLTQPPSVSVAPGETATITCGGNLIGAYLVHWYQQKPGQAPVLV | 212 |
| VL | IYDDVDRPARIPERFSGSNSGNTATLTISRVEAGDEADYYCQVWDH | |
| | DTNTMVFGGGTKLTVL | |
| BDG38_076_ | QMQLVESGGGVVQPGRSLRLSCAASGFAFRTYGMHWVRQAPGKG | 213 |
| VH | LEWVAVIWDDGSATHYADSVKGRFTITRDNSKNTLNLQMNSLRAE | |
| | DTAVYYCVRAPQWYLTAEAFDLWGQGTMVTVSS | |
| BDG38_076_ | SYVLTQPPSVSVAPGETASITCGGNMIGGYLVHWYQQKPGQAPLLV | 214 |
| VL | IYDDVDRPARIPERFSGSNSGNTATLTISRVEAGDEADYYCQVWDH | |
| | DTNHMVFGGGTKLTVL | |
| BDG38_077_ | QMQLVESGGGVVQPGRSLTLSCAASGFAFRTYGMHWVRQAPGKG | 215 |
| VH | LEWVGVIWYDGSATHYADSVKGRFTITRDNSKNTLNLQMNSLRAE | |
| | DTAVYYCVRAPQWYLTAEAFDLWGQGTMVTVSS | |
| BDG38_077_ | SYVLTQPPSVSVAPGETATITCGGALIGSRLVHWYQQKPGQAPVLVI | 216 |
| VL | YDDSDRPSRIPERFSGSNIGNTATLTISDVEAGDEADYYCQVWDHSS | |
| | NTYVFGGGTKLTVL | |
| BDG38_078_ | QMQLVESGGGVVQPGRSLRLSCAASGFEFRTYGMHWVRQAPGKG | 217 |
| VH | LEWLGVIWYDGSNTHYADSVKGRFTITRDNSKNTLNLQMNSLRVE | |
| | DTAVYYCVRAPQWYLTAEAFDLWGQGTMVTVSS | |
| BDG38_078_ | SYILTQPPSVSVAPGETATITCGGNLIGARLVHWYQQKPGQAPVLVI | 218 |
| VL | YDDSDRPSRIPERFSGSNIGNTATLTIERVEAGDEADYYCQVWDYYS | |
| | DHMVFGGGTKLTVL | |
| BDG38_080_ | QMQLVESGGGVVQPGRSLRLSCAASGFAFRTYGMHWVRQAPGKG | 221 |
| VH | LEWVAVIWYDGSATHYADSVKGRFTITRDNSKNTLNLQMNSLRAE | |
| | DTAVYYCVRAPQWYLSAEAFDLWGQGTMVTVSS | |
| BDG38_080_ | SYVLTQPPSVSVAPGETATITCGGNLIGAYLVHWYQQKPGQAPVLV | 222 |
| VL | IYDDSDRPDRIPERFSGSNSGNTATLTISRVEAGDEADYYCQVWDH | |
| | NTNHMVFGGGTKLTVL | |
| BDG38_081_ | QMQLVESGGGVVQPGRSLRLSCAASGFAFRTYGMHWVRQAPGKG | 223 |
| VH | LEWVAVIWDDGSNTHYADSVKGRFTITRDNSKNTLNLQMNSLRAE | |
| | DTAVYYCVRAPQWYLTAEAFDLWGQGTMVTVSS | |
| BDG38_081_ | SYVLTQPPSVSVAPGQTARITCGGNMIGAYLVHWYQQKPGQAPLL | 224 |
| VL | VIYDDVDRPDRIPERFSGSNSGNTATLTISRVEAGDEADYYCQVWD | |
| | HNTQQMVFGGGTKLTVL | |
| BDG38_082_ | QMQLVESGGGVVQPGRSLRLSCAASGFAFRTYGMHWVRQAPGKG | 225 |
| VH | LEWVAVIWDDGSNTHYADSVKGRFTITRDNSKNTLNLQMNSLRAE | |
| | DTAVYYCVRAPQWYLTAEAFDLWGQGTMVTVSS | |
| BDG38_082_ | SYVLTQPPSVSVAPGETATITCGGNMIGGYLVHWYQQKPGQAPVL | 226 |
| VL | VIYDDVDRPDRIPERFSGSNSGNTATLTISRVEAGDEADYYCQVWD | |
| | HDTNQVVFGGGTKLTVL | |
| BDG38_083_ | QMQLVESGGGVVQPGRSLTLSCAASGFEFRTYGMHWVRQAPGKG | 227 |
| VH | LEWVGVIWDDGSNTHYADSVKGRFTITRDNSKNTLNLQMNSLRAE | |
| | DTAVYYCARAPQWYLTAEAFDLWGQGTMVTVSS | |
| BDG38_083_ | SYILTQPPSVSVAPGQTATITCGGNLIGAKLVHWYQQKPGQAPVLVI | 228 |
| VL | YDDSDRPSRIPERFSGSNIGNTATLTISDVEEGDEADYYCQVWDYSS | |
| | DTMVFGGGTKLTVL | |
| BDG38_084_ | QMQLVESGGGVVQPGRSLRLSCAASGFAFRTYGMHWVRQAPGKG | 229 |
| VH | LEWVAVIWDDGSNTHYADSVKGRFTITRDNSKNTLNLQMNSLRAE | |
| | DTAVYYCVRAPQWYLTAEAFDLWGQGTMVTVSS | |
| BDG38_084_ | SYVLTQPPSVSVAPGETATITCGGNLIGAYLVHWYQQKPGQAPVLV | 230 |
| VL | VYDDVDRPDRIPERFSGSNSGNTATLTISRVEAGDEADYYCQVWDH | |
| | STNTMVFGGGTKLTVL | |
| BDG38_085_ | QMQLVESGGGVVQPGRSLTLSCAASGFEFRTYGMHWVRQAPGKG | 231 |
| VH | LEWVGVIWYDGSNTHYADSVKGRFTITRDNSKNTLNLQMNSLRVE | |
| | DTAVYYCVRAPQWYLTAEAFDLWGQGTMVTVSS | |
| BDG38_085_ | SYVLTQPPSVSVAPGETATITCGGNLIGARLVHWYQQKPGQAPVLV | 232 |
| VL | IYDDSDRPSRIPERFSGSNIGNTATLTIERVEAGDEADYYCQVWDYS | |
| | SNSYVFGGGTKLTVL | |
| BDG38_086_ | QMQLVESGGGVVQPGRSLRLSCAASGFEFRTYGMHWVRQAPGKG | 233 |
| VH | LEWVGVIWYDGSNTHYADSVKGRFTITRDNSKNTLNLQMNSLRAE | |
| | DTAVYYCVRAPQWYLTAEAFDLWGQGTMVTVSS | |
| BDG38_086_ | SYVLTQPPSVSVAPGQTARITCGGNLIGARLVHWYQQKPGQAPVLV | 234 |
| VL | VYDDSDRPSRIPERFSGSNIGNTATLTISDVEEGDEADYYCQVWDYS | |
| | SNTYVFGGGTKLTVL | |
| BDG38_087_ | QMQLVESGGGVVQPGRSLRLSCAASGFAFRTYGMHWVRQAPGKG | 235 |
| VH | LEWVAVIWDDGSNTHYADSVKGRFTITRDNSKNTLNLQMNSLRAE | |
| | DTAVYYCVRAPQWYLTAEAFDLWGQGTMVTVSS | |
| BDG38_087_ | SYVLTQPPSVSVAPGQTATITCGGNMIGAYLVHWYQQKPGQAPVL | 236 |
| VL | VIYDDVDRPDRIPERFSGSNSGNTATLTISRVEAGDEADYYCQVWD | |
| | HNSNQMVFGGGTKLTVL | |
| BDG38_088_ | QMQLVESGGGVVQPGRSLTLSCAASGFEFRTYGMHWVRQAPGKG | 237 |
| VH | LEWVGVIWYDGSNTHYADSVKGRFTITRDNSKNTLNLQMNSLRVE | |
| | DTAVYYCVRAPQWYLTAEAFDLWGQGTMVTVSS | |
| BDG38_088_ | SYVLTQPPSVSVAPGETATITCGGNLIGARLVHWYQQKPGQAPVLV | 238 |
| VL | IYDDSDRPSRIPERFSGSNIGNTATLTISDVEAGDEADYYCQVWDYS | |
| | SNTYVFGGGTKLTVL | |
| BDG38_089_ | QMQLVESGGGVVQPGRSLRLSCAASGFEFRTYGMHWVRQAPGKG | 239 |
| VH | LEWVGVIWYDGSATHYADSVKGRFTITRDNSKNTLNLQMNSLRVE | |
| | DTAVYYCVRAPQWYLTAEAFDLWGQGTMVTVSS | |
| BDG38_089_ | SYILTOPPSVSVAPGETATITCGGNLIGARLVHWYQQKPGQAPVLVI | 240 |
| VL | YDDSDRPSRIPERFSGSNIGNTATLTISRVEEGDEADYYCQVWDHSS | |
| | NHYVFGGGTKLTVL | |
| BDG38_090_ | QMQLVESGGGVVQPGRSLTLSCAASGFEFRTYGMHWVRQAPGKG | 241 |
| VH | LEWLGVIWDDGSNTHYADSVKGRFTITRDNSKNTLNLQMNSLRVE | |
| | DTAVYYCARAPQWYLTAEAFDLWGQGTMVTVSS | |
| BDG38_090_ | SYILTQPPSVSVAPGETARITCGGNLIGARLVHWYQQKPGQAPVLV | 242 |
| VL | VYDDSDRPSRIPERFSGSNIGNTATLTIEDVEEGDEADYYCQVWDY | |
| | YSDHMVFGGGTKLTVL | |
| BDG38_091_ | QMQLVESGGGVVQPGRSLTLSCAASGFEFRTYGMHWVRQAPGKG | 243 |
| VH | LEWLGVIWYDGSNTHYADSVKGRFTITRDNSKNTLNLQMNSLRVE | |
| | DTAVYYCVRAPQWYLTAEAFDLWGQGTMVTVSS | |
| BDG38_091_ | SYILTQPPSVSVAPGETATITCGGNLIGSRLVHWYQQKPGQAPVLVI | 244 |
| VL | YDDSDRPSRIPERFSGSNIGNTATLTISDVEAGDEADYYCQVWDHYS | |
| | NHYVFGGGTKLTVL | |
| BDG38_092_ | QMQLVESGGGVVQPGRSLTLSCAASGFEFRTYGMHWVRQAPGKG | 245 |
| VH | LEWLAVIWDDGSNTHYADSVKGRFTITRDNSKNTLNLQMNSLRVE | |
| | DTAVYYCVRAPQWYLTAEAFDLWGQGTMVTVSS | |
| BDG38_092_ | SYVLTQPPSVSVAPGETATITCGGNLIGARLVHWYQQKPGQAPVLV | 246 |
| VL | IYDDSDRPSRIPERFSGSNIGNTATLTISDVEEGDEADYYCQVWDYY | |
| | SDHMVFGGGTKLTVL | |
| BDG38_093_ | QMQLVESGGGVVQPGRSLTLSCAASGFEFRTYGMHWVRQAPGKG | 247 |
| VH | LEWVGVIWYDGSNTHYADSVKGRFTITRDNSKNTLNLQMNSLRAE | |
| | DTAVYYCVRAPQWYLTAEAFDLWGQGTMVTVSS | |
| BDG38_093_ | SYVLTQPPSVSVAPGQTARITCGGNLIGARLVHWYQQKPGQAPVLV | 248 |
| VL | IYDDSDRPSRIPERFSGSNIGNTATLTIERVEEGDEADYYCQVWDYS | |
| | ADSYVFGGGTKLTVL | |
| BDG38_095_ | QMQLVESGGGVVQPGRSLTLSCAASGFEFRTYGMHWVRQAPGKG | 251 |
| VH | LEWVGVIWYDGSATHYADSVKGRFTITRDNSKNTLNLQMNSLRAE | |
| | DTAVYYCVRAPQWYLTAEAFDLWGQGTMVTVSS | |
| BDG38_095_ | SYVLTQPPSVSVAPGQTATITCGGALIGSRLVHWYQQKPGQAPVLV | 252 |
| VL | VYDDSDRPSRIPERFSGSNIGNTATLTISRVEEGDEADYYCQVWDYY | |
| | SDSYVFGGGTKLTVL | |
| BDG38_096_ | QMQLVESGGGVVQPGRSLRLSCAASGFEFRTYGMHWVRQAPGKG | 253 |
| VH | LEWVAVIWDDGSNTHYADSVKGRFTITRDNSKNTLNLQMNSLRVE | |
| | DTAVYYCVRAPQWYLTAEAFDLWGQGTMVTVSS | |
| BDG38_096_ | SYILTQPPSVSVAPGETARITCGGNLIGARLVHWYQQKPGQAPVLVI | 254 |
| VL | YDDSDRPSRIPERFSGSNIGNTATLTISRVEEGDEADYYCQVWDYSS | |
| | DSMVFGGGTKLTVL | |
| BDG38_097_ | QMQLVESGGGVVQPGRSLRLSCAASGFEFRTYGMHWVRQAPGKG | 255 |
| VH | LEWLGVIWYDGSATHYADSVKGRFTITRDNSKNTLNLQMNSLRAE | |
| | DTAVYYCVRAPQWYLTAEAFDLWGQGTMVTVSS | |
| BDG38_097_ | SYILTQPPSVSVAPGETATITCGGNLIGARLVHWYQQKPGQAPVLVI | 256 |
| VL | YDDSDRPSRIPERFSGSNIGNTATLTIERVEEGDEADYYCQVWDYYS | |
| | DHYVFGGGTKLTVL | |
| BDG38_098_ | QMQLVESGGGVVQPGRSLTLSCAASGFEFRTYGMHWVRQAPGKG | 257 |
| VH | LEWVAVIWDDGSNTVYADSVKGRFTITRDNSKNTLNLQMNSLRVE | |
| | DTAVYYCVRAPQWYLTAEAFDLWGQGTMVTVSS | |
| BDG38_098_ | SYVLTQPPSVSVAPGQTATITCGGNLIGARLVHWYQQKPGQAPVLV | 258 |
| VL | IYDDSDRPSRIPERFSGSNIGNTATLTISRVEAGDEADYYCQVWDYS | |
| | SDSMVFGGGTKLTVL | |
| BDG38_099_ | QMQLVESGGGVVQPGRSLRLSCAASGFEFRTYGMHWVRQAPGKG | 259 |
| VH | LEWVGVIWYDGSNTHYADSVKGRFTITRDNSKNTLNLQMNSLRAE | |
| | DTAVYYCVRAPQWYLTAEAFDLWGQGTMVTVSS | |
| BDG38_099_ | SYILTQPPSVSVAPGETARITCGGNLIGARLVHWYQQKPGQAPVLVI | 260 |
| VL | YDDSDRPSRIPERFSGSNIGNTATLTIEDVEEGDEADYYCQVWDYSS | |
| | DSYVFGGGTKLTVL | |
| BDG38_100_ | QMQLVESGGGVVQPGRSLRLSCAASGFEFRTYGMHWVRQAPGKG | 261 |
| VH | LEWVGVIWYDGSNTHYADSVKGRFTITRDNSKNTLNLQMNSLRVE | |
| | DTAVYYCVRAPQWYLTAEAFDLWGQGTMVTVSS | |
| BDG38_100_ | SYVLTQPPSVSVAPGETARITCGGNLIGARLVHWYQQKPGQAPVLV | 262 |
| VL | IYDDSDRPSRIPERFSGSNIGNTATLTIERVEEGDEADYYCQVWDYY | |
| | SNSYVFGGGTKLTVL | |
| BDG38_101_ | QMQLVESGGGVVQPGRSLRLSCAASGFEFRTYGMHWVRQAPGKG | 263 |
| VH | LEWVGVIWYDGSATHYADSVKGRFTITRDNSKNTLNLQMNSLRVE | |
| | DTAVYYCVRAPQWYLTAEAFDLWGQGTMVTVSS | |
| BDG38_101 | SYILTQPPSVSVAPGETATITCGGNLIGARLVHWYQQKPGQAPVLVI | 264 |
| VL | YDDSDRPSRIPERFSGSNIGNTATLTISRVEEGDEADYYCQVWDYSS | |
| | NTYVFGGGTKLTVL | |
| BDG38_102_ | QMQLVESGGGVVQPGRSLRLSCAASGFEFRTYGMHWVRQAPGKG | 265 |
| VH | LEWVGVIWYDGSNTHYADSVKGRFTITRDNSKNTLNLQMNSLRAE | |
| | DTAVYYCVRAPQWYLTAEAFDLWGQGTMVTVSS | |
| BDG38_102_ | SYVLTQPPSVSVAPGETATITCGGNLIGARLVHWYQQKPGQAPVLV | 266 |
| VL | VYDDSDRPSRIPERFSGSNIGNTATLTIEDVEAGDEADYYCQVWDY | |
| | SSNTYVFGGGTKLTVL | |
| BDG38_103_ | QMQLVESGGGVVQPGRSLRLSCAASGFEFRTYGMHWVRQAPGKG | 267 |
| VH | LEWVGVIWYDGSNTHYADSVKGRFTITRDNSKNTLNLQMNSLRVE | |
| | DTAVYYCVRAPQWYLTAEAFDLWGQGTMVTVSS | |
| BDG38_103_ | SYVLTQPPSVSVAPGQTATITCGGNLIGARLVHWYQQKPGQAPVLV | 268 |
| VL | VYDDSDRPSRIPERFSGSNIGNTATLTISDVEEGDEADYYCQVWDY | |
| | YSNSYVFGGGTKLTVL | |
| BDG38_104_ | QMQLVESGGGVVQPGRSLTLSCAASGFEFRTYGMHWVRQAPGKG | 269 |
| VH | LEWVGVIWYDGSATHYADSVKGRFTITRDNSKNTLNLQMNSLRAE | |
| | DTAVYYCVRAPQWYLTAEAFDLWGQGTMVTVSS | |
| BDG38_104_ | SYILTQPPSVSVAPGETATITCGGNLIGARLVHWYQQKPGQAPVLVI | 270 |
| VL | YDDSDRPSRIPERFSGSNIGNTATLTIEDVEEGDEADYYCQVWDYYS | |
| | DSYVFGGGTKLTVL | |
| BDG38_105_ | QMQLVESGGGVVQPGRSLTLSCAASGFEFRTYGMHWVRQAPGKG | 271 |
| VH | LEWVGVIWYDGSNTHYADSVKGRFTITRDNSKNTLNLQMNSLRVE | |
| | DTAVYYCVRAPQWYLTAEAFDLWGQGTMVTVSS | |
| BDG38_105_ | SYVLTQPPSVSVAPGETATITCGGNLIGARLVHWYQQKPGQAPVLV | 272 |
| VL | IYDDSDRPSRIPERFSGSNIGNTATLTIERVEAGDEADYYCQVWDYY | |
| | SNSYVFGGGTKLTVL | |
| BDG38_106_ | QMQLVESGGGVVQPGRSLTLSCAASGFEFRTYGMHWVRQAPGKG | 273 |
| VH | LEWVAVIWDDGSNTVYADSVKGRFTITRDNSKNTLNLQMNSLRVE | |
| | DTAVYYCVRAPQWYLTAEAFDLWGQGTMVTVSS | |
| BDG38_106_ | SYILTQPPSVSVAPGQTATITCGGNLIGSRLVHWYQQKPGQAPVLVI | 274 |
| VL | YDDSDRPSRIPERFSGSNIGNTATLTIEDVEEGDEADYYCQVWDHYS | |
| | DHMVFGGGTKLTVL | |
| BDG38_107_ | QMQLVESGGGVVQPGRSLTLSCAASGFEFRTYGMHWVRQAPGKG | 275 |
| VH | LEWVGVIWYDGSNTHYADSVKGRFTITRDNSKNTLNLQMNSLRAE | |
| | DTAVYYCVRAPQWYLTAEAFDLWGQGTMVTVSS | |
| BDG38_107_ | SYILTQPPSVSVAPGQTATITCGGNLIGARLVHWYQQKPGQAPVLVI | 276 |
| VL | YDDSDRPSRIPERFSGSNIGNTATLTIERVEEGDEADYYCQVWDYSS | |
| | DSYVFGGGTKLTVL | |
| BDG38_108_ | QMQLVESGGGVVQPGRSLRLSCAASGFEFRTYGMHWVRQAPGKG | 277 |
| VH | LEWVGVIWYDGSNTHYADSVKGRFTITRDNSKNTLNLQMNSLRAE | |
| | DTAVYYCVRAPQWYLTAEAFDLWGQGTMVTVSS | |
| BDG38_108_ | SYILTQPPSVSVAPGETATITCGGNLIGARLVHWYQQKPGQAPVLVI | 278 |
| VL | YDDSDRPSRIPERFSGSNIGNTATLTISDVEEGDEADYYCQVWDYYS | |
| | NSYVFGGGTKLTVL | |
| BDG38_109_ | QMQLVESGGGVVQPGRSLRLSCAASGFEFRTYGMHWVRQAPGKG | 279 |
| VH | LEWVGVIWYDGSNTHYADSVKGRFTITRDNSKNTLNLQMNSLRAE | |
| | DTAVYYCVRAPQWYLTAEAFDLWGQGTMVTVSS | |
| BDG38_109_ | SYILTQPPSVSVAPGQTATITCGGNLIGARLVHWYQQKPGQAPVLVI | 280 |
| VL | YDDSDRPSRIPERFSGSNIGNTATLTIERVEEGDEADYYCQVWDYSS | |
| | NSYVFGGGTKLTVL | |
| BDG38_110_ | QMQLVESGGGVVQPGRSLRLSCAASGFEFRTYGMHWVRQAPGKG | 281 |
| VH | LEWVAVIWDDGSNTHYADSVKGRFTITRDNSKNTLNLQMNSLRAE | |
| | DTAVYYCVRAPQWYLTAEAFDLWGQGTMVTVSS | |
| BDG38_110_ | SYILTQPPSVSVAPGETTRITCGGNLIGAKLVHWYQQKPGQAPVLVI | 282 |
| VL | YDDSDRPSRIPERFSGSNIGNTATLTIEDVEAGDEADYYCQVWDYSS | |
| | NHMVFGGGTKLTVL | |
| BDG38_111_ | QMQLVESGGGVVQPGRSLTLSCAASGFEFRTYGMHWVRQAPGKG | 283 |
| VH | LEWVGVIWDDGSATHYADSVKGRFTITRDNSKNTLNLQMNSLRVE | |
| | DTAVYYCVRAPQWYLTAEAFDLWGQGTMVTVSS | |
| BDG38_111_ | SYVLTQPPSVSVAPGETATITCGGALIGARLVHWYQQKPGQAPVLV | 284 |
| VL | VYDDSDRPSRIPERFSGSNIGNTATLTISRVEAGDEADYYCQVWDY | |
| | YANSYVFGGGTKLTVL | |
| BDG38_112_ | QMQLVESGGGVVQPGRSLRLSCAASGFEFRTYGMHWVRQAPGKG | 285 |
| VH | LEWLGVIWYDGSNTHYADSVKGRFTITRDNSKNTLNLQMNSLRAE | |
| | DTAVYYCVRAPQWYLTAEAFDLWGQGTMVTVSS | |
| BDG38_112_ | SYILTQPPSVSVAPGQTATITCGGNLIGARLVHWYQQKPGQAPVLVI | 286 |
| VL | YDDSDRPSRIPERFSGSNIGNTATLTIERVEEGDEADYYCQVWDYSS | |
| | DTYVFGGGTKLTVL | |
| BDG38_113_ | QMQLVESGGGVVQPGRSLTLSCAASGFEFRTYGMHWVRQAPGKG | 287 |
| VH | LEWVGVIWYDGSNTHYADSVKGRFTITRDNSKNTLNLQMNSLRAE | |
| | DTAVYYCVRAPQWYLTAEAFDLWGQGTMVTVSS | |
| BDG38_113_ | SYVLTQPPSVSVAPGETATITCGGNLIGARLVHWYQQKPGQAPVLV | 288 |
| VL | IYDDSDRPSRIPERFSGSNIGNTATLTIEDVEEGDEADYYCQVWDYS | |
| | SDSYVFGGGTKLTVL | |
| BDG38_114_ | QMQLVESGGGVVQPGRSLTLSCAASGFEFRTYGMHWVRQAPGKG | 289 |
| VH | LEWVGVIWYDGSNTHYADSVKGRFTITRDNSKNTLNLQMNSLRVE | |
| | DTAVYYCVRAPQWYLTAEAFDLWGQGTMVTVSS | |
| BDG38_114_ | SYILTQPPSVSVAPGQTATITCGGNLIGARLVHWYQQKPGQAPVLVI | 290 |
| VL | YDDSDRPSRIPERFSGSNIGNTATLTIEDVEEGDEADYYCQVWDYSA | |
| | NSYVFGGGTKLTVL | |
| BDG38_115_ | QMQLVESGGGVVQPGRSLTLSCAASGFEFRTYGMHWVRQAPGKG | 291 |
| VH | LEWLGVIWYDGSNTHYADSVKGRFTITRDNSKNTLNLQMNSLRAE | |
| | DTAVYYCVRAPQWYLTAEAFDLWGQGTMVTVSS | |
| BDG38_115_ | SYILTQPPSVSVAPGQTATITCGGNLIGARLVHWYQQKPGQAPVLVI | 292 |
| VL | YDDSDRPSRIPERFSGSNSGNTATLTISDVEEGDEADYYCQVWDYSS | |
| | NTYVFGGGTKLTVL | |
| BDG38_116_ | QMQLVESGGGVVQPGRSLTLSCAASGFEFRTYGMHWVRQAPGKG | 293 |
| VH | LEWVAVIWDDGSNTHYADSVKGRFTITRDNSKNTLNLQMNSLRVE | |
| | DTAVYYCVRAPQWYLTAEAFDLWGQGTMVTVSS | |
| BDG38_116_ | SYVLTQPPSVSVAPGQTATITCGGNLIGARLVHWYQQKPGQAPVLV | 294 |
| VL | IYDDSDRPSRIPERFSGSNIGNTATLTIERVEAGDEADYYCQVWDYY | |
| | SDTMVFGGGTKLTVL | |
| BDG38_117_ | QMQLVESGGGVVQPGRSLTLSCAASGFEFRTYGMHWVRQAPGKG | 295 |
| VH | LEWVGVIWDDGSNTHYADSVKGRFTITRDNSKNTLNLQMNSLRAE | |
| | DTAVYYCARAPQWYLTAEAFDLWGQGTMVTVSS | |
| BDG38_117_ | SYVLTQPPSVSVAPGETATITCGGNLIGAKLVHWYQQKPGQAPVLV | 296 |
| VL | IYDDSDRPSRIPERFSGSNIGNTATLTIEDVEAGDEADYYCQVWDYS | |
| | SDTMVFGGGTKLTVL | |
| BDG38_118_ | QMQLVESGGGVVQPGRSLRLSCAASGFEFRTYGMHWVRQAPGKG | 297 |
| VH | LEWVGVIWYDGSATHYADSVKGRFTITRDNSKNTLNLQMNSLRVE | |
| | DTAVYYCVRAPQWYLTAEAFDLWGQGTMVTVSS | |
| BDG38_118_ | SYILTQPPSVSVAPGETATITCGGNLIGARLVHWYQQKPGQAPVLVI | 298 |
| VL | YDDSDRPSRIPERFSGSNIGNTATLTISDVEEGDEADYYCQVWDYSS | |
| | DHYVFGGGTKLTVL | |
| BDG38_119_ | QMQLVESGGGVVQPGRSLRLSCAASGFEFRTYGMHWVRQAPGKG | 299 |
| VH | LEWVGVIWYDGSNTHYADSVKGRFTITRDNSKNTLNLQMNSLRAE | |
| | DTAVYYCVRAPQWYLTAEAFDLWGQGTMVTVSS | |
| BDG38_119_ | SYILTQPPSVSVAPGETATITCGGNLIGARLVHWYQQKPGQAPVLVI | 300 |
| VL | YDDSDRPSRIPERFSGSNIGNTATLTISRVEEGDEADYYCQVWDYSS | |
| | NTYVFGGGTKLTVL | |
| BDG38_120_ | QMQLVESGGGVVQPGRSLRLSCAASGFEFRTYGMHWVRQAPGKG | 301 |
| VH | LEWVAVIWDDGSNTHYADSVKGRFTITRDNSKNTLNLQMNSLRAE | |
| | DTAVYYCVRAPQWYLTAEAFDLWGQGTMVTVSS | |
| BDG38_120_ | SYILTQPPSVSVAPGETATITCGGNLIGARLVHWYQQKPGQAPVLVI | 302 |
| VL | YDDSDRPSRIPERFSGSNSGNTATLTISDVEAGDEADYYCQVWDYS | |
| | SDHMVFGGGTKLTVL | |
| BDG38_121_ | QMQLVESGGGVVQPGRSLRLSCAASGFEFRTYGMHWVRQAPGKG | 303 |
| VH | LEWVGVIWYDGSNTHYADSVKGRFTITRDNSKNTLNLQMNSLRAE | |
| | DTAVYYCVRAPQWYLTAEAFDLWGQGTMVTVSS | |
| BDG38_121_ | SYILTQPPSVSVAPGETATITCGGNLIGARLVHWYQQKPGQAPVLVI | 304 |
| VL | YDDSDRPSRIPERFSGSNIGNTATLTISRVEAGDEADYYCQVWDYSS | |
| | NTYVFGGGTKLTVL | |
| BDG38_122_ | QMQLVESGGGVVQPGRSLRLSCAASGFEFRTYGMHWVRQAPGKG | 305 |
| VH | LEWVAVIWDDGSNTHYADSVKGRFTITRDNSKNTLNLQMNSLRAE | |
| | DTAVYYCVRAPQWYLTAEAFDLWGQGTMVTVSS | |
| BDG38_122_ | SYILTQPPSVSVAPGETATITCGGNLIGARLVHWYQQKPGQAPVLVI | 306 |
| VL | YDDSDRPSRIPERFSGSNIGNTATLTISRVEEGDEADYYCQVWDYSS | |
| | DSYVFGGGTKLTVL | |
| BDG38_123_ | QMQLVESGGGVVQPGRSLRLSCAASGFEFRTYGMHWVRQAPGKG | 307 |
| VH | LEWVAVIWDDGSNTHYADSVKGRFTITRDNSKNTLNLQMNSLRAE | |
| | DTAVYYCVRAPQWYLTAEAFDLWGQGTMVTVSS | |
| BDG38_123_ | SYILTQPPSVSVAPGETATITCGGNLIGARLVHWYQQKPGQAPVLVI | 308 |
| VL | YDDSDRPSRIPERFSGSNIGNTATLTISDVEAGDEADYYCQVWDYSS | |
| | DTYVFGGGTKLTVL | |
| BDG38_124_ | QMQLVESGGGVVQPGRSLRLSCAASGFEFRTYGMHWVRQAPGKG | 309 |
| VH | LEWVGVIWDDGSNTHYADSVKGRFTITRDNSKNTLNLQMNSLRAE | |
| | DTAVYYCVRAPQWYLTAEAFDLWGQGTMVTVSS | |
| BDG38_124_ | SYILTQPPSVSVAPGETATITCGGNLIGARLVHWYQQKPGQAPVLVI | 310 |
| VL | YDDSDRPSRIPERFSGSNIGNTATLTISDVEAGDEADYYCQVWDYSS | |
| | DTYVFGGGTKLTVL | |
| BDG38_125_ | QMQLVESGGGVVQPGRSLRLSCAASGFEFRTYGMHWVRQAPGKG | 311 |
| VH | LEWVAVIWDDGSNTHYADSVKGRFTITRDNSKNTLNLQMNSLRAE | |
| | DTAVYYCVRAPQWYLTAEAFDLWGQGTMVTVSS | |
| BDG38_125_ | SYVLTQPPSVSVAPGETATITCGGNLIGARLVHWYQQKPGQAPVLV | 312 |
| VL | IYDDSDRPSRIPERFSGSNIGNTATLTIERVEEGDEADYYCQVWDYS | |
| | SDHMVFGGGTKLTVL | |
| BDG38_126_ | QMQLVESGGGVVQPGRSLRLSCAASGFEFRTYGMHWVRQAPGKG | 313 |
| VH | LEWVGVIWDDGSNTHYADSVKGRFTITRDNSKNTLNLQMNSLRAE | |
| | DTAVYYCVRAPQWYLTAEAFDLWGQGTMVTVSS | |
| BDG38_126_ | SYILTQPPSVSVAPGQTARITCGGNLIGAKLVHWYQQKPGQAPVLVI | 314 |
| VL | YDDSDRPSRIPERFSGSNIGNTATLTIERVEEGDEADYYCQVWDYSS | |
| | DHMVFGGGTKLTVL | |
| BDG38_127_ | QMQLVESGGGVVQPGRSLTLSCAASGFEFRTYGMHWVRQAPGKG | 315 |
| VH | LEWVGVIWYDGSATHYADSVKGRFTITRDNSKNTLNLQMNSLRAE | |
| | DTAVYYCARAPQWYLTAEAFDLWGQGTMVTVSS | |
| BDG38_127_ | SYILTQPPSVSVAPGETATITCGGNLIGAKLVHWYQQKPGQAPVLVI | 316 |
| VL | YDDSDRPSRIPERFSGSNIGNTATLTISDVEAGDEADYYCQVWDYYS | |
| | DTYVFGGGTKLTVL | |
| BDG38_128_ | QMQLVESGGGVVQPGRSLTLSCAASGFEFRTYGMHWVRQAPGKG | 317 |
| VH | LEWVAVIWDDGSNTHYADSVKGRFTITRDNSKNTLNLQMNSLRVE | |
| | DTAVYYCVRAPQWYLTAEAFDLWGQGTMVTVSS | |
| BDG38_128_ | SYILTQPPSVSVAPGETATITCGGNLIGAKLVHWYQQKPGQAPVLVI | 318 |
| VL | YDDSDRPSRIPERFSGSNSGNTATLTISDVEEGDEADYYCQVWDYY | |
| | ADTMVFGGGTKLTVL | |
| BDG38_129_ | QMQLVESGGGVVQPGRSLTLSCAASGFEFRTYGMHWVRQAPGKG | 319 |
| VH | LEWVGVIWDDGSNTHYADSVKGRFTITRDNSKNTLNLQMNSLRVE | |
| | DTAVYYCARAPQWYLTAEAFDLWGQGTMVTVSS | |
| BDG38_129_ | SYILTQPPSVSVAPGQTATITCGGNLIGAKLVHWYQQKPGQAPVLVI | 320 |
| VL | YDDSDRPSRIPERFSGSNIGNTATLTISDVEEGDEADYYCQVWDYSS | |
| | DHMVFGGGTKLTVL | |
| BDG38_130_ | QMQLVESGGGVVQPGRSLTLSCAASGFAFRTYGMHWVRQAPGKG | 321 |
| VH | LEWVAVIWDDGSNTHYADSVKGRFTITRDNSKNTLNLQMNSLRVE | |
| | DTAVYYCVRAPQWYLTAEAFDLWGQGTMVTVSS | |
| BDG38_130_ | SYILTOPPSVSVAPGETATITCGGNLIGARLVHWYQQKPGQAPVLVI | 322 |
| VL | YDDSDRPSRIPERFSGSNIGNTATLTIERVEAGDEADYYCQVWDYSS | |
| | NSYVFGGGTKLTVL | |
| BDG38_131_ | QMQLVESGGGVVQPGRSLTLSCAASGFEFRTYGMHWVRQAPGKG | 323 |
| VH | LEWVAVIWDDGSNTHYADSVKGRFTITRDNSKNTLNLQMNSLRAE | |
| | DTAVYYCVRAPQWYLTAEAFDLWGQGTMVTVSS | |
| BDG38_131_ | SYILTQPPSVSVAPGETATITCGGNLIGAKLVHWYQQKPGQAPVLVI | 324 |
| VL | YDDSDRPSRIPERFSGSNIGNTATLTISDVEEGDEADYYCQVWDYYS | |
| | NTMVFGGGTKLTVL | |
| BDG38_132_ | QMQLVESGGGVVQPGRSLRLSCAASGFEFRTYGMHWVRQAPGKG | 325 |
| VH | LEWVGVIWDDGSNTHYADSVKGRFTITRDNSKNTLNLQMNSLRAE | |
| | DTAVYYCVRAPQWYLTAEAFDLWGQGTMVTVSS | |
| BDG38_132_ | SYILTQPPSVSVAPGETATITCGGNLIGARLVHWYQQKPGQAPVLVI | 326 |
| VL | YDDSDRPSRIPERFSGSNIGNTATLTISRVEAGDEADYYCQVWDYSS | |
| | DHMVFGGGTKLTVL | |
| BDG38_133_ | QMQLVESGGGVVQPGRSLRLSCAASGFEFRTYGMHWVRQAPGKG | 327 |
| VH | LEWVAVIWDDGSNTHYADSVKGRFTITRDNSKNTLNLQMNSLRAE | |
| | DTAVYYCVRAPQWYLTAEAFDLWGQGTMVTVSS | |
| BDG38_133_ | SYILTQPPSVSVAPGETATITCGGNLIGARLVHWYQQKPGQAPVLVI | 328 |
| VL | YDDSDRPSRIPERFSGSNIGNTATLTISRVEAGDEADYYCQVWDYSS | |
| | DTYVFGGGTKLTVL | |
| BDG38_134_ | QMQLVESGGGVVQPGQSLRLSCAASGFEFRTYGMHWVRQAPGKG | 329 |
| VH | LEWLAVIWYDGSNTHYADSVKGRFTITRDNSKNTLNLQMNSLRAE | |
| | DTAVYYCVRAPQWYNSAEAFDLWGQGTMVTVSS | |
| BDG38_134_ | SYVLTQPPSVSVAPGQTARITCGGNLIGARLVHWYQQKPGQAPVLV | 330 |
| VL | IYDDSDRPSHIPERFSGSNSGNTATLTISRVEAGDEADYYCQVWDYS | |
| | ADTMVFGGGTKLTVL | |
| BDG38_135_ | QMQLVESGGGVVQPGQSLRLSCAASGFAFRTYGMHWVRQAPGKG | 331 |
| VH | LEWLAVIWYDGSNTHYADSVKGRFTITRDNSKNTLNLQMNSLRAE | |
| | DTAVYYCVRAPQWYNSAEAFDLWGQGTMVTVSS | |
| BDG38_135_ | SYVLTQPPSVSVAPGQTARITCGGNLIGARLVHWYQQKPGQAPVLV | 332 |
| VL | VYDDSDRPSHIPERFSGSNSGNTATLTISRVEAGDEADYYCQVWDH | |
| | SADTMVFGGGTKLTVL | |
| BDG38_136_ | QMQLVESGGGVVQPGRSLRLSCAASGFEFRTYGMHWVRQAPGKG | 333 |
| VH | LEWLAVIWYDGSNTHYADSVKGRFTITRDNSKNTLNLQMNSLRAE | |
| | DTAVYYCVRAPQWYNSAEAFDLWGQGTMVTVSS | |
| BDG38_136_ | SYVLTQPPSVSVAPGQTARITCGGNLIGAKLVHWYQQKPGQAPVLV | 334 |
| VL | IYDDSDRPSHIPERFSGSNSGNTATLTISRVEAGDEADYYCQVWDYS | |
| | SDTMVFGGGTKLTVL | |
| BDG38_137_ | QMQLVESGGGVVQPGQSLRLSCAASGFEFRTYGMHWVRQAPGKG | 335 |
| VH | LEWVAVIWDDGSNTHYADSVKGRFTITRDNSKNTLNLQMNSLRAE | |
| | DTAVYYCVRAPQWYLTAEAFDLWGQGTMVTVSS | |
| BDG38_137_ | SYVLTQPPSVSVAPGETATITCGGNLIGARLVHWYQQKPGQAPVLV | 336 |
| VL | IYDDSDRPSHIPERFSGSNSGNTATLTISRVEAGDEADYYCQVWDYS | |
| | ADTMVFGGGTKLTVL | |
| BDG38_139_ | QMQLVESGGGVVQPGRSLRLSCAASGFAFRTYGMHWVRQAPGKG | 339 |
| VH | LEWVAVIWDDGSNTHYADSVKGRFTITRDNSKNTLNLQMNSLRAE | |
| | DTAVYYCVRAPQWYLSAEAFDLWGQGTMVTVSS | |
| BDG38_139_ | SYVLTQPPSVSVAPGETATITCGGNMIGAYLVHWYQQKPGQAPLLV | 340 |
| VL | IYDDVDRPARIPERFSGSNSGNTATLTISRVEAGDEADYYCQVWDH | |
| | NSDHMVFGGGTKLTVL | |
| BDG38_140_ | QMQLVESGGGVVQPGRSLTLSCAASGFEFRTYGMHWVRQAPGKG | 341 |
| VH | LEWVGVIWDDGSNTHYADSVKGRFTITRDNSKNTLNLQMNSLRVE | |
| | DTAVYYCARAPQWYLTAEAFDLWGQGTMVTVSS | |
| BDG38_140_ | SYILTQPPSVSVAPGETATITCGGNLIGAKLVHWYQQKPGQAPVLVI | 342 |
| VL | YDDSDRPSRIPERFSGSNIGNTATLTIEDVEAGDEADYYCQVWDYS | |
| | ANHMVFGGGTKLTVL | |
| BDG38_141_ | QMQLVESGGGVVQPGRSLRLSCAASGFEFRTYGMHWVRQAPGKG | 343 |
| VH | LEWVAVIWDDGSNTHYADSVKGRFTITRDNSKNTLNLQMNSLRAE | |
| | DTAVYYCVRAPQWYLTAEAFDLWGQGTMVTVSS | |
| BDG38_141_ | SYILTQPPSVSVAPGETATITCGGALIGSRLVHWYQQKPGQAPVLVI | 344 |
| VL | YDDSDRPSRIPERFSGSNIGNTATLTISDVEEGDEADYYCQVWDYYS | |
| | NHMVFGGGTKLTVL | |
| BDG38_142_ | QMQLVESGGGVVQPGRSLTLSCAASGFEFRTYGMHWVRQAPGKG | 345 |
| VH | LEWVAVIWDDGSNTHYADSVKGRFTITRDNSKNTLNLQMNSLRAE | |
| | DTAVYYCVRAPQWYLTAEAFDLWGQGTMVTVSS | |
| BDG38_142_ | SYILTQPPSVSVAPGQTATITCGGNLIGAKLVHWYQQKPGQAPVLV | 346 |
| VL | VYDDSDRPSRIPERFSGSNIGNTATLTIERVEEGDEADYYCQVWDY | |
| | YSHTMVFGGGTKLTVL | |
| BDG38_143_ | QMQLVESGGGVVQPGRSLRLSCAASGFEFRTYGMHWVRQAPGKG | 347 |
| VH | LEWVGVIWDDGSATHYADSVKGRFTITRDNSKNTLNLQMNSLRVE | |
| | DTAVYYCVRAPQWYLTAEAFDLWGQGTMVTVSS | |
| BDG38_143_ | SYILTQPPSVSVAPGQTATITCGGALIGARLVHWYQQKPGQAPVLVI | 348 |
| VL | YDDSDRPSRIPERFSGSNIGNTATLTISRVEEGDEADYYCQVWDYSS | |
| | NTYVFGGGTKLTVL | |

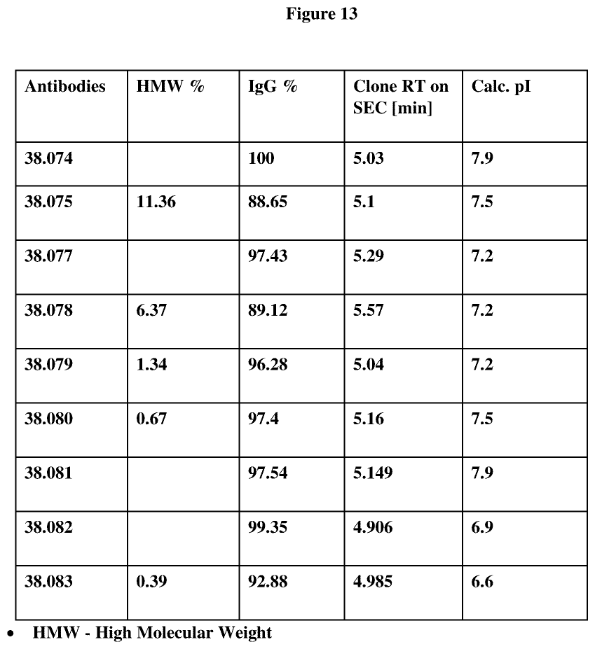

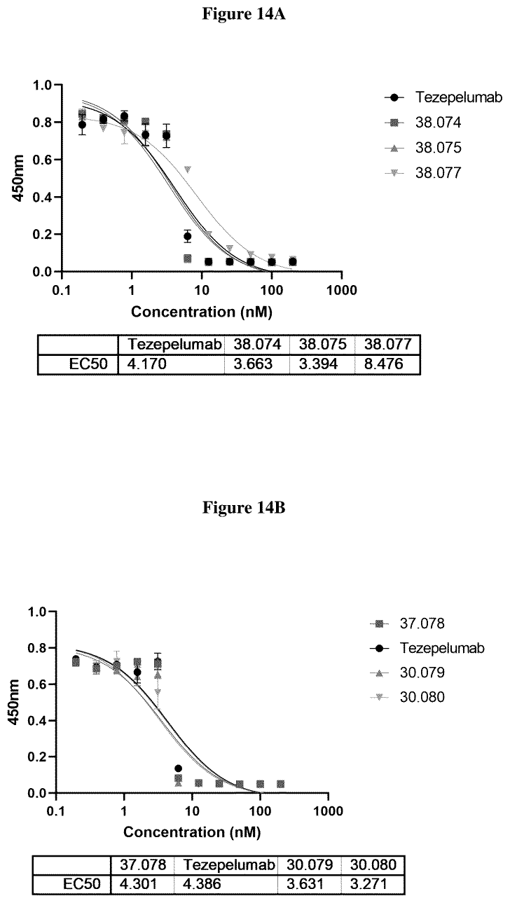

Data presented below demonstrates biochemical and functional properties for some of the dual binding antibodies disclosed herein. FIG. 13 shows retention time and calculated pI for some of the dual binding antibodies disclosed herein, demonstrating that BDG antibodies are monodispersed and highly homogenic.

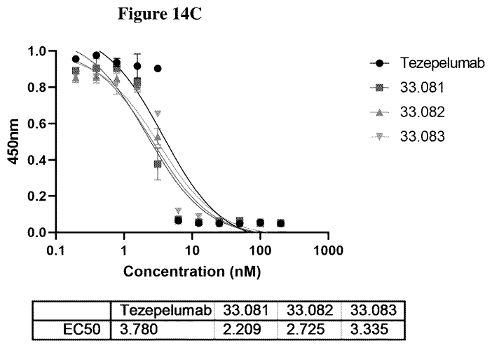

FIGS. 14A-14C show competitive ELISA of some of the dual binding antibodies and Tezepelumab with TSLP. ELISA plates were coated overnight at 4° C. with 50 ng/well Tezepelumab. BDG antibodies were double diluted and pre-incubated with a constant concentration of 7 nM TSLP-His for 1 hour at room temperature. After blocking and washing steps, BDG antibodies-TSLP mix were added to the plates, incubated for 10 minutes and washed again, followed by a 30 minute incubation with anti-HIS. BDG antibodies exhibited similar IC50 values and complete competition with Tezepelumab at the TSLP epitope.

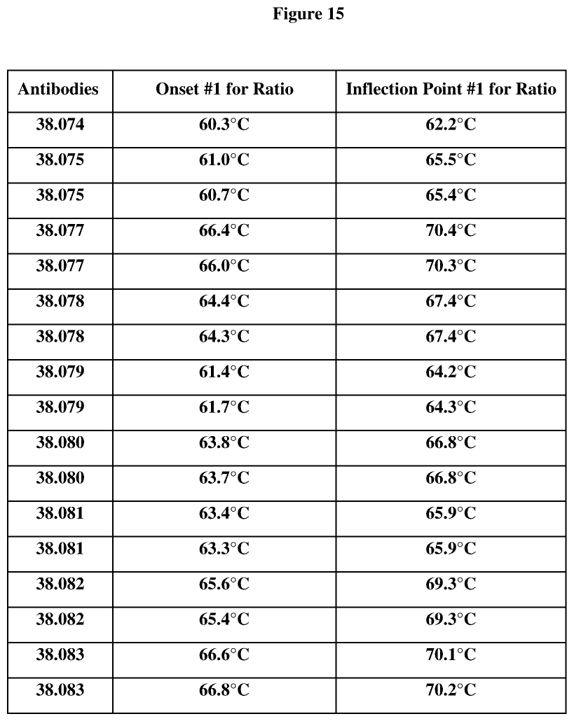

FIG. 15 shows nanoscale differential scanning fluorimetry (nanoDSF) analysis of some of the dual binding antibodies disclosed herein. All BDG antibodies were analyzed to test stability over a range of thermal changes using nanoDSF (See, Table 11 below). Antibodies with a Tm>60° C. were selected to further characterizations.

TABLE 11
SEC and DSF summary table. SEC column presents Area
Under the Curve (AUC) peak ratio of monodispersed
BDG antibodies fraction. DSF columns present T-onset
and Tm values of the different BDG antibodies.

| | | DSF 1st repetion | | DSF 2nd repetition | |
|---|---|---|---|---|---|
| Antibody# | SEC (%) | T-onset | Tm-1 | T-onset | Tm-1 |
| BDG38.074 | 100 | 60.3 | 62.2 | | |
| BDG38.075 | 88.65 | 61 | 65.5 | 60.7 | 65.4 |
| BDG38.076 | 91.98 | | | | |
| BDG38.077 | 97.43 | 66.4 | 70.4 | 66 | 70.3 |
| BDG38.078 | 89.12 | 64.3 | 67.4 | 64.4 | 67.4 |
| BDG38.079 | 96.28 | 61.4 | 64.2 | 61.7 | 64.3 |
| BDG38.080 | 97.4 | 63.7 | 66.8 | 63.8 | 66.8 |
| BDG38.081 | 97.54 | 63.3 | 65.9 | 63.4 | 65.9 |
| BDG38.082 | 99.35 | 65.6 | 69.3 | 65.4 | 69.3 |
| BDG38.083 | 92.88 | 66.6 | 70.1 | 66.8 | 70.2 |
| BDG38.084 | 96.7 | 20.0° C. | 49.6° C. | 23.7° C. | 48.4° C. |
| BDG38.085 | 90 | | 51.1° C. | | 51.5° C. |
| BDG38.086 | 68 | | 51.3° C. | | 51.1° C. |
| BDG38.087 | | | | | |
| BDG38.088 | 84.3 | 65.2° C. | 71.5° C. | | 51.6° C. |
| BDG38.089 | 92.34 | 61.0° C. | 71.2° C. | 60.8° C. | 71.3° C. |
| BDG38.090 | 96.6 | 63.5° C. | 69.7° C. | 63.5° C. | 69.7° C. |
| BDG38.091 | 86.78 | 57.6° C. | 72.0° C. | 58.0° C. | 72.1° C. |
| BDG38.092 | 95.8 | 64.5° C. | 68.4° C. | 63.9° C. | 68.2° C. |
| BDG38.093 | 82.33 | | 49.6° C. | | 49.6° C. |
| BDG38.094 | 96.15 | 21.4° C. | 49.7° C. | 62.4° C. | 65.3° C. |
| BDG38.095 | 98.01 | 64.6° C. | 68.2° C. | 53.0° C. | 53.5° C. |
| BDG38.096 | 97 | 63.5° C. | 66.9° C. | 64.2° C. | 67.0° C. |
| BDG38.097 | 68 | 65.0° C. | 68.5° C. | 64.7° C. | 68.5° C. |
| BDG38.098 | 95.8 | 63.4° C. | 66.1° C. | 62.8° C. | 66.2° C. |
| BDG38.099 | 75.2 | | 49.4° C. | | 58.3° C. |
| BDG38.100 | 84 | | 50.8° C. | | 51.2° C. |
| BDG38.101 | 97 | 63.6° C. | 70.6° C. | 64.5° C. | 70.5° C. |
| BDG38.102 | 70.6 | | 58.9° C. | 65.4° C. | 68.8° C. |
| BDG38.103 | 78.26 | 51.0° C. | 51.2° C. | 65.5° C. | 69.2° C. |
| BDG38.104 | 76.36 | 65.0° C. | 69.3° C. | 65.4° C. | 69.3° C. |
| BDG38.105 | 74.87 | | 51.8° C. | 64.7° C. | 71.2° C. |
| BDG38.106 | 96.8 | 63.9° C. | 67.5° C. | 64.3° C. | 67.6° C. |
| BDG38.107 | 69.19 | | 49.3° C. | 49.1° C. | 49.2° C. |
| BDG38.108 | 75.47 | 42.2° C. | 42.9° C. | | 51.5° C. |
| BDG38.109 | 56 | | 51.2° C. | | 51.4° C. |
| BDG38.110 | 77.15 | 27.7° C. | 56.8° C. | 26.0° C. | 57.2° C. |
| BDG38.111 | 92 | 23.4° C. | 55.4° C. | 43.9° C. | 54.9° C. |
| BDG38.112 | 71.97 | | 49.5° C. | | 49.3° C. |
| BDG38.113 | 71 | | 49.6° C. | | 50.0° C. |
| BDG38.114 | 67.9 | | 59.1° C. | | 52.0° C. |
| BDG38.115 | 72.41 | | 56.8° C. | 57.4° C. | 71.5° C. |
| BDG38.116 | 88 | 63.8° C. | 67.6° C. | 64.3° C. | 67.9° C. |
| BDG38.117 | 96 | 64.6° C. | 69.7° C. | 63.9° C. | 69.7° C. |
| BDG38.118 | 90.5 | 64.5° C. | 69.6° C. | 64.8° C. | 69.7° C. |
| BDG38.119 | 63.19 | | 51.7° C. | | 59.9° C. |
| BDG38.120 | 82 | 28.3° C. | 55.8° C. | 62.9° C. | 66.8° C. |
| BDG38.121 | 51 | | 49.9° C. | | 50.1° C. |
| BDG38.122 | 67 | 58.5° C. | 58.9° C. | | 59.0° C. |
| BDG38.123 | 59 | | 50.6° C. | | 50.5° C. |
| BDG38.124 | 69 | 57.5° C. | 58.5° C. | 56.2° C. | 58.3° C. |
| BDG38.125 | 85.47 | 63.9° C. | 67.2° C. | 63.6° C. | 67.1° C. |
| BDG38.126 | 93 | 63.9° C. | 67.3° C. | 64.0° C. | 67.3° C. |
| BDG38.127 | 89 | 62.9° C. | 69.3° C. | 62.8° C. | 69.3° C. |
| BDG38.128 | 81 | 63.4° C. | 67.4° C. | 63.2° C. | 67.4° C. |
| BDG38.129 | 95.98 | 63.8° C. | 70.1° C. | 63.4° C. | 70.1° C. |
| BDG38.130 | 38.5 | | 60.2° C. | | 60.5° C. |
| BDG38.131 | 71.5 | 64.2° C. | 68.8° C. | 64.4° C. | 68.8° C. |
| BDG38.132 | 94.35 | 63.3° C. | 67.1° C. | 63.6° C. | 67.1° C. |
| BDG38.133 | 61.3 | | 50.8° C. | | 50.4° C. |
| BDG38.134 | 93.57 | 63.5° C. | 67.4° C. | 63.6° C. | 67.4° C. |
| BDG38.135 | 92.78 | 62.6° C. | 65.6° C. | 62.6° C. | 65.6° C. |
| BDG38.136 | 93.38 | 63.8° C. | 67.3° C. | 63.6° C. | 67.3° C. |
| BDG38.137 | 77.8 | 63.4° C. | 67.9° C. | 62.7° C. | 67.8° C. |
| BDG38.138 | 94.8 | 20.0° C. | 50.4° C. | 62.8° C. | 66.0° C. |
| BDG38.139 | 90 | 61.6° C. | 64.8° C. | 62.0° C. | 64.8° C. |
| BDG38.140 | 82.2 | 63.6° C. | 70.6° C. | 20.0° C. | 50.9° C. |
| BDG38.141 | 73.25 | 20.0° C. | 55.2° C. | 20.0° C. | 56.1° C. |
| BDG38.142 | 80 | 63.8° C. | 67.8° C. | 63.8° C. | 67.7° C. |
| BDG38.143 | 74 | 21.3° C. | 57.5° C. | | 56.9° C. |

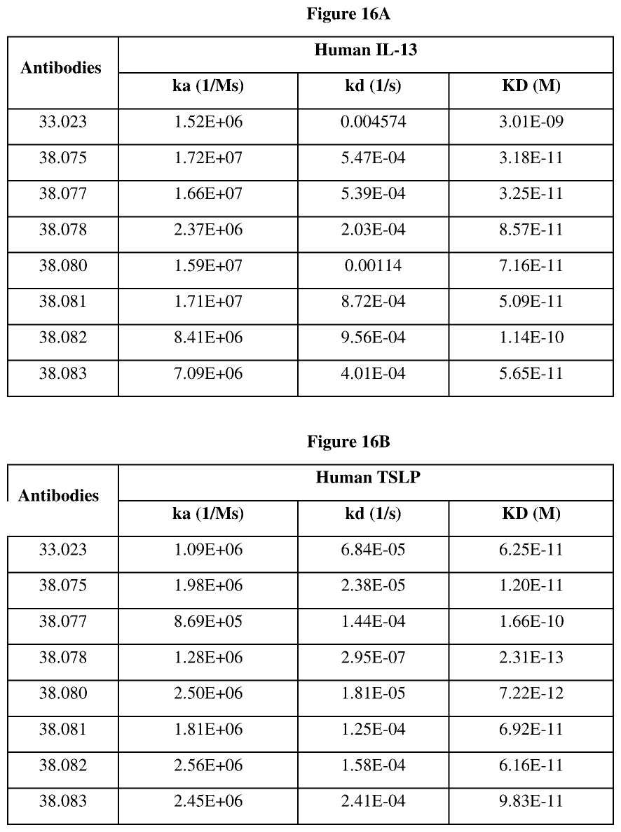

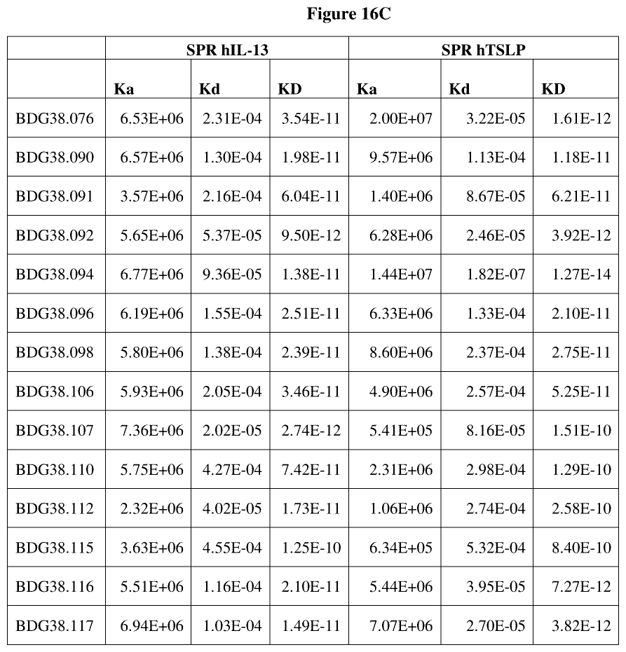

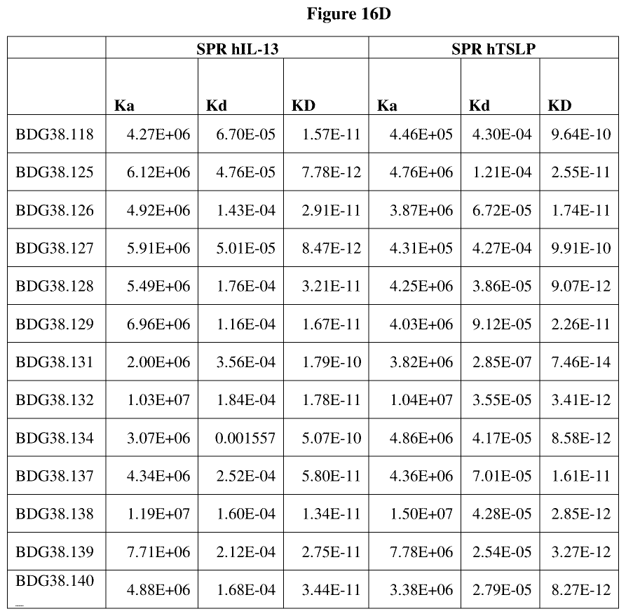

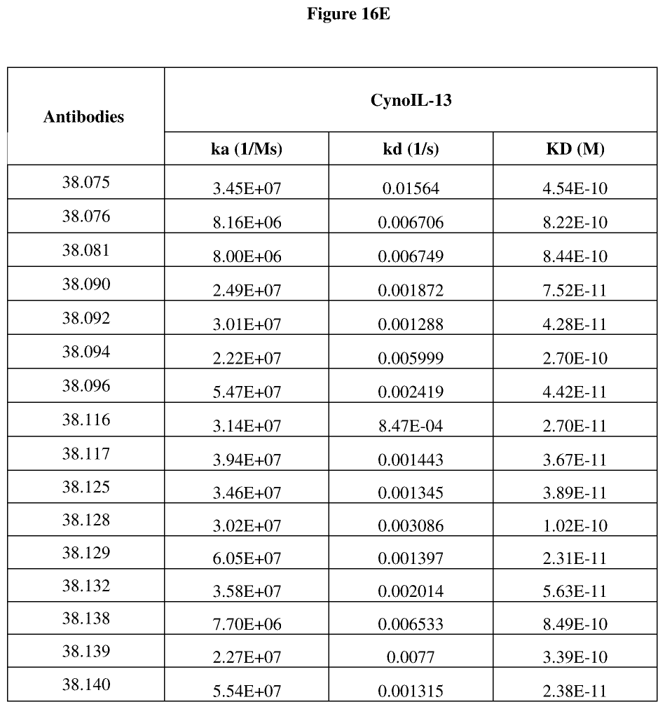

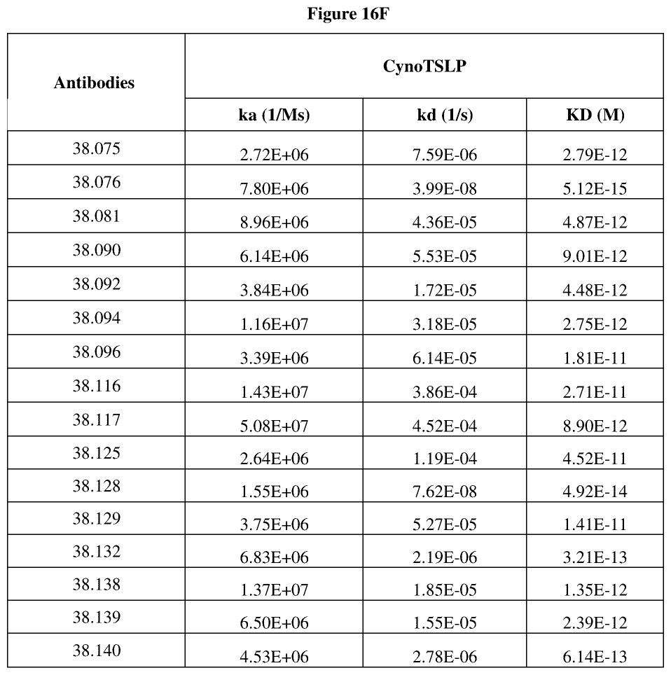

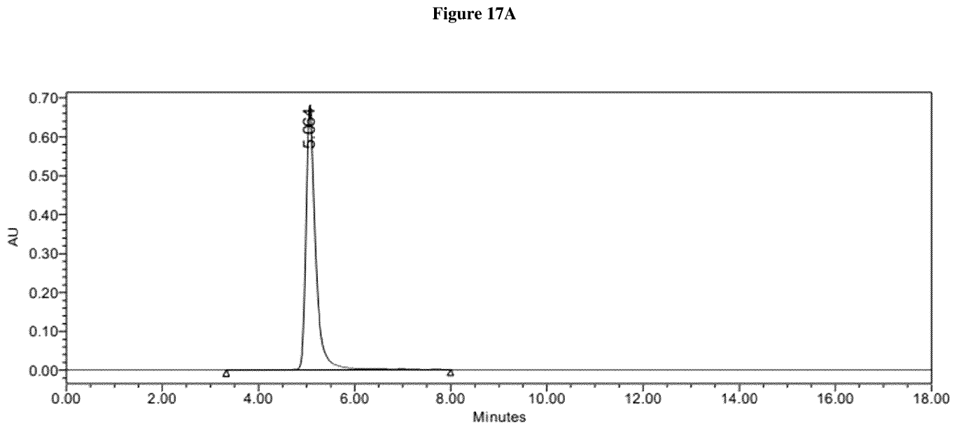

FIGS. 16A-16F show the results of SPR (Surface Plasmon Resonance) analysis for some of the dual binding antibodies disclosed herein for human/cyno IL-13 and TSLP. SPR analysis was performed to assess the kinetics of BDG antibodies binding to TSLP and IL-13. SPR data indicates dissociation constants of double digit pM for BDG antibodies binding to IL-13 and TSLP.

Table 12 presents IC50 inhibition values of CD23 expression on monocytes and IC50 inhibition values of Thymus and activation-regulated chemokine (TARC) secretion by PBMCs. This data demonstrates that each BDG dual antibody can inhibit both IL-13 and TSLP functions in human PBMCs.

TABLE 12
PBMC Functional Assay Summary Table.

PBMC Functional Assay

| Antibody# | IC50 CD23 [nM] | IC50 TARC [nM] |
|---|---|---|
| BDG38.074 | 0.1578 | 2.771 |
| BDG38.075 | 0.09752 | 4.168 |
| BDG38.076 | 0.3078 | 3.206 |
| BDG38.077 | 0.01245 | 2.466 |
| BDG38.078 | 0.1252 | 6.516 |
| BDG38.079 | 0.07179 | 1.961 |
| BDG38.080 | 0.2011 | 0.7904 |
| BDG38.081 | 0.03386 | 5.929 |
| BDG38.082 | 0.03908 | 1.577 |
| BDG38.083 | 0.02146 | 11.37 |
| BDG38.090 | 0.006556 | 0.007555 |
| BDG38.092 | 0.06747 | 5.876 |
| BDG38.094 | 0.1724 | 0.3556 |
| BDG38.096 | 0.2504 | 23.41 |
| BDG38.106 | 0.1616 | NaN |
| BDG38.112 | 0.05937 | 0.1039 |
| BDG38.116 | 0.08366 | 59.36 |
| BDG38.117 | 0.1207 | 5.40E+15 |
| BDG38.125 | 0.0527 | NaN |
| BDG38.126 | 0.0868 | 2.575 |
| BDG38.128 | 0.01492 | NaN |
| BDG38.129 | 0.029 | NaN |
| BDG38.131 | 0.07 | NaN |
| BDG38.132 | 0.43 | 22.39 |
| BDG38.135 | 0.4075 | 0.8042 |
| BDG38.137 | 0.3728 | 0.8233 |
| BDG38.138 | 0.1638 | 2.358 |
| BDG38.139 | 0.012 | — |
| BDG38.140 | 0.146 | — |

*NaN—stands for values that could not be determined.

Table 13 presents the IC50 values obtained from competitive ELISA with Tezepelumab. The data demonstrates that BDG dual antibodies compete with Tezepelumab for TSLP binding.

TABLE 13
IC50 Competition ELISA Values Summary Table.

| Antibody# | IC50(nM) vs anti-TSLP benchmark |
|---|---|
| BDG38.074 | 3.66 |
| BDG38.075 | 3.94 |
| BDG38.076 | 5.382 |
| BDG38.077 | 8.47 |
| BDG38.078 | 4.3 |
| BDG38.079 | 3.63 |
| BDG38.080 | 3.27 |
| BDG38.081 | 2.2 |
| BDG38.082 | 2.7 |
| BDG38.083 | 3.33 |
| BDG38.090 | 7.31 |
| BDG38.092 | 7.382 |
| BDG38.096 | 5.871 |
| BDG38.106 | 10.09 |
| BDG38.112 | 29.14 |
| BDG38.116 | 5.123 |
| BDG38.117 | 6.137 |
| BDG38.125 | 8.442 |
| BDG38.126 | 5.953 |
| BDG38.128 | 5.378 |
| BDG38.129 | 5.494 |
| BDG38.132 | 9.203 |
| BDG38.137 | 10.89 |
| BDG38.139 | 9.571 |
| BDG38.140 | 5.628 |

Table 14 presents data from a TSLP functional inhibition assay using MUTZ5 cells (human B cell precursor leukemia cells). IC50 values of STATS phosphorylation inhibition are presented in pM, and the R2 column presents goodness of fit values. These results demonstrate that BDG dual antibodies can inhibit TSLP function in the MUTZ5 cell line expressing the native TSLP-receptor heterocomplex (TLSP-R and IL-7R).

TABLE 14
TSLP Functional Inhibition Assay in MUTZ5 cell line.

| Antibody# | IC50 [pM] | R² |
|---|---|---|
| BDG38.074 | 46.74 | 0.925 |
| BDG38.075 | 43.86 | 0.9919 |
| BDG38.076 | 30.14 | 0.9322 |
| BDG38.077 | 304.1 | 0.7798 |
| BDG38.078 | 337.5 | 0.9148 |
| BDG38.079 | 21.84 | 0.8359 |
| BDG38.080 | 34.33 | 0.9534 |
| BDG38.081 | 38.74 | 0.9556 |
| BDG38.086 | 830.1 | 0.01605 |
| BDG38.090 | 43.35 | 0.2951 |
| BDG38.091 | 0.5069 | 0.43 |
| BDG38.092 | 33.05 | 0.5048 |
| BDG38.094 | 27.16 | 0.9719 |
| BDG38.096 | 5.44E−05 | 0.6063 |
| BDG38.098 | 68.84 | 0.07219 |
| BDG38.107 | 0.5081 | 0.3199 |
| BDG38.110 | 0.2951 | 0.8273 |
| BDG38.112 | 11383 | 0.05737 |
| BDG38.115 | 300.3 | 0.1132 |
| BDG38.117 | 0.3628 | 0.5132 |
| BDG38.118 | 0.6884 | 0.4463 |
| BDG38.120 | 0.2315 | 0.7447 |
| BDG38.125 | 8.74E−05 | 0.7192 |
| BDG38.126 | 0.04215 | 0.7997 |
| BDG38.127 | 0.1433 | 0.5647 |
| BDG38.128 | 0.004796 | 0.639 |
| BDG38.129 | 0.6765 | 0.5382 |
| BDG38.132 | 0.3858 | 0.737 |
| BDG38.134 | 32.04 | 0.7965 |
| BDG38.137 | 1.855 | 0.08069 |
| BDG38.138 | 45.24 | 0.9313 |
| BDG38.139 | 88.95 | 0.8952 |
| BDG38.140 | 2.211 | 0.5831 |

Table 15 shows the results of a functional inhibition assay in IL-4/IL-13 reporter HEK 293 cells. IC50 values of STATE phosphorylation inhibition is presented in nM. This data demonstrated that BDG dual antibodies can inhibit IL-13 function in reporter HEK cell line cells expressing the IL-13-receptor heterocomplex (IL-13Rα1 and IL-4Rα).

TABLE 15
IL-13 Functional Inhibition Assay in IL-
4/IL-13 Reporter HEK 293 cell line.

| Antibody# | HEK-IL-13 [nM] |
|---|---|
| BDG38.074 | 0.02407 |
| BDG38.075 | 0.06912 |
| BDG38.076 | 0.04938 |
| BDG38.077 | 0.05055 |
| BDG38.078 | 0.05079 |
| BDG38.079 | 0.0253 |
| BDG38.080 | 0.05923 |
| BDG38.081 | 0.04828 |
| BDG38.082 | 0.04295 |
| BDG38.083 | 0.04194 |
| BDG38.084 | 0.0419 |
| BDG38.085 | 0.02355 |
| BDG38.086 | 0.0174 |
| BDG38.087 | — |
| BDG38.088 | 0.04861 |
| BDG38.089 | 0.07497 |
| BDG38.090 | 0.1035 |
| BDG38.091 | 0.08321 |
| BDG38.092 | 0.1197 |
| BDG38.093 | 0.04018 |
| BDG38.094 | 0.04873 |
| BDG38.095 | 0.08224 |
| BDG38.096 | 0.1505 |
| BDG38.097 | 0.04645 |
| BDG38.098 | 0.08838 |
| BDG38.099 | 0.06009 |
| BDG38.100 | 0.08009 |
| BDG38.101 | 0.04035 |
| BDG38.102 | 0.04211 |
| BDG38.103 | 0.084 |
| BDG38.104 | 0.03805 |
| BDG38.105 | 0.1341 |
| BDG38.106 | 0.01134 |
| BDG38.107 | 0.09427 |
| BDG38.108 | 0.06595 |
| BDG38.109 | 0.06295 |
| BDG38.110 | 0.02988 |
| BDG38.111 | 0.05454 |
| BDG38.112 | 0.03665 |
| BDG38.113 | 0.02843 |
| BDG38.114 | 0.09887 |
| BDG38.115 | 0.06638 |
| BDG38.116 | 0.1799 |
| BDG38.117 | 0.08006 |
| BDG38.118 | 0.08044 |
| BDG38.119 | 0.06617 |
| BDG38.120 | 0.2649 |
| BDG38.121 | 0.05618 |
| BDG38.122 | 0.07895 |
| BDG38.123 | 0.04405 |
| BDG38.124 | 0.05233 |
| BDG38.125 | 0.05618 |
| BDG38.126 | 0.07895 |
| BDG38.127 | 0.04405 |
| BDG38.128 | 0.05233 |
| BDG38.129 | 0.07253 |
| BDG38.130 | 0.06463 |
| BDG38.131 | 0.0443 |
| BDG38.132 | 0.08986 |
| BDG38.133 | 0.02895 |
| BDG38.134 | 0.2711 |
| BDG38.135 | 0.4058 |
| BDG38.136 | 0.1509 |
| BDG38.137 | 0.1951 |
| BDG38.138 | 0.02862 |
| BDG38.139 | 0.04644 |
| BDG38.140 | 0.04303 |
| BDG38.141 | 0.08244 |
| BDG38.142 | 0.05843 |
| BDG38.143 | 0.04382 |

Antibody BDG38.074

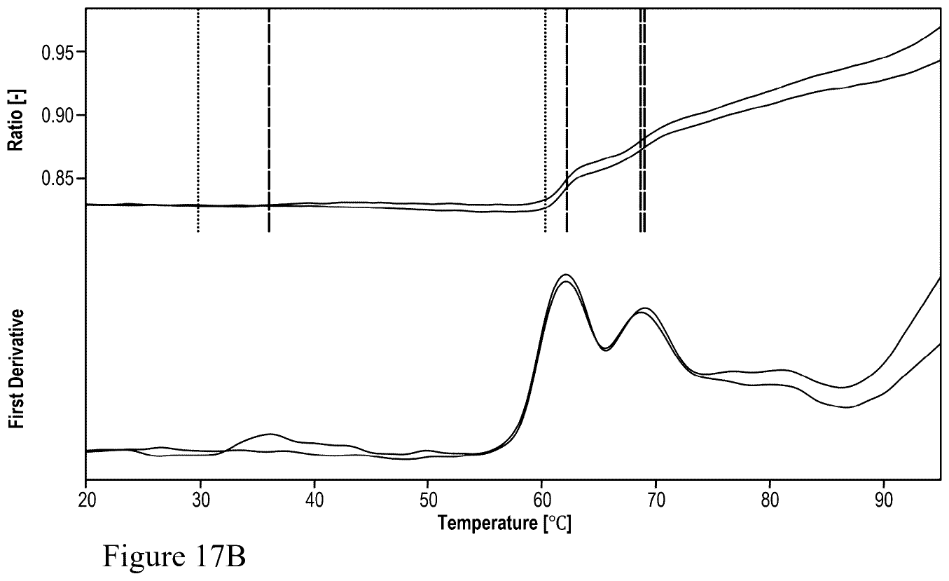

The size exclusion chromatography (SEC) scans and nano-differential scanning fluorimetry (DSF) analysis of the melting point for antibody BDG38.074 are shown in FIGS. 17A and 17B. SEC analysis (FIG. 17A) was performed using BioResolve SEC mAb Column, 200 Å, 2.5 μm, 4.6×300 mm at 0.5 ml/min in PBS as a mobile phase and analyzed at 280 nm. BDG38.074 shows a predominant monodisperse peak with undetectable aggregates. DSF analysis (FIG. 17B) was performed using nanoDSF at a 1° C./min from 20-95° C.

NanoDSF is monitoring the thermal unfolding of BDG38.074 according to the intrinsic fluorescence change at 350 and 330 nm. The top half of the graph in FIG. 17B shows the fluorescence ratio of 350 nm/330 nm as a function of temperature and the bottom half shows the first derivative as a function of temperature. BDG38.074 was analyzed at 0.5 mg/ml in PBS showed to have a T-onset of 60.3° C. and Tm of 62.2° C. suggesting a relatively stable fold.

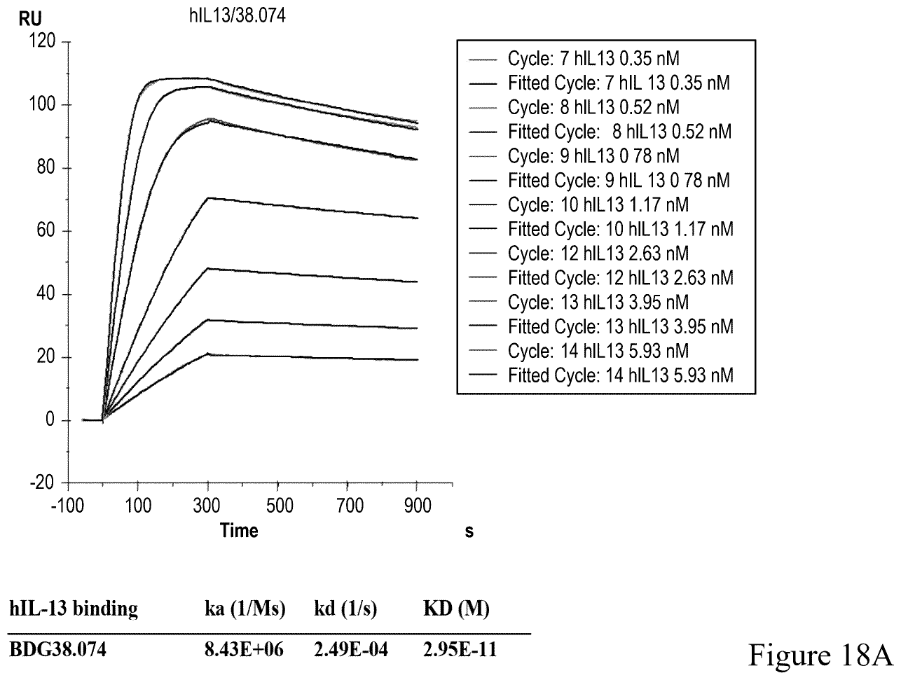

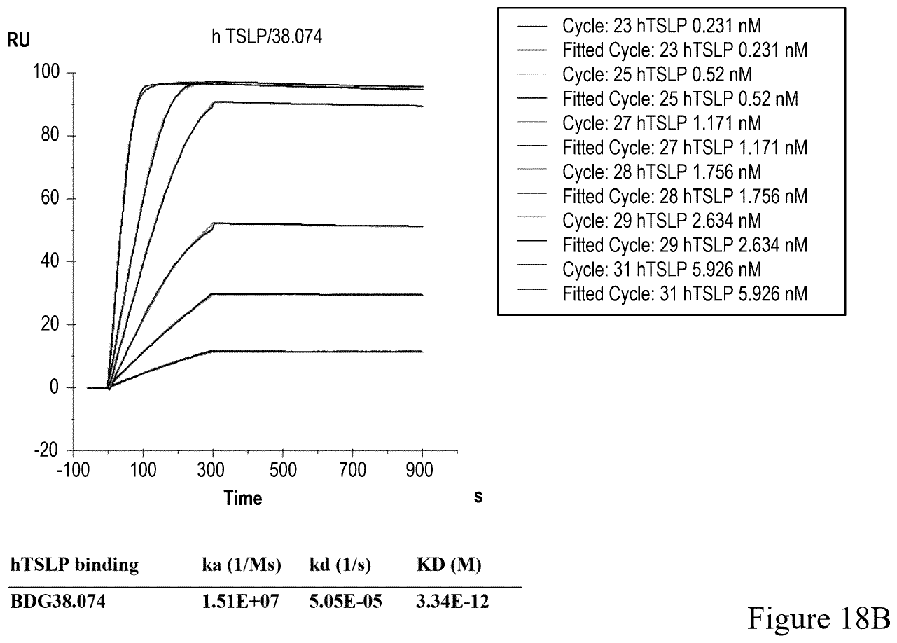

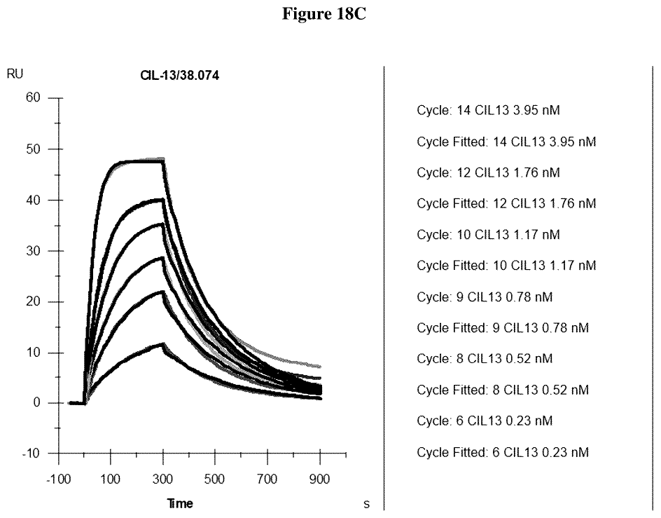

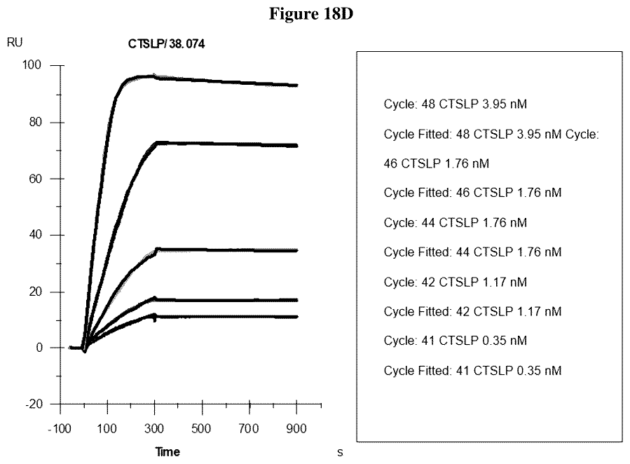

The binding affinities of antibody BDG38.074 to human/cyno IL-13 and human/cyno TSLP are shown in FIGS. 18A-18D. Surface Plasmon Resonance (SPR) analysis of BDG38.074 binding to human IL-13 (FIG. 18A), human TSLP (FIG. 18B), cyno IL-13 (FIG. 18C) and cyno TSLP (FIG. 18D) using BiacoreT200. CMS chip was coated with human antibody capture kit to obtain 3000-5000 RU and the antibody, served as the capture, was injected at a flow rate of 10 ul/ml to obtain 250-350 RU. Human/cynoTSLP and human/cynoIL-13 served as analytes in a concentration range of 30-0.153 nM. Contact time 300 sec and dissociation time 600 sec at a flow rate of 30 ul/min BDG38.074 shows a high affinity to all cytokines with KD values for hIL-13: 2.95E-11M, for hTSLP: 3.34E-12 M, for cyno IL-13: 7.33E-10 M and for cyno TSLP: 4.16E-12 M.

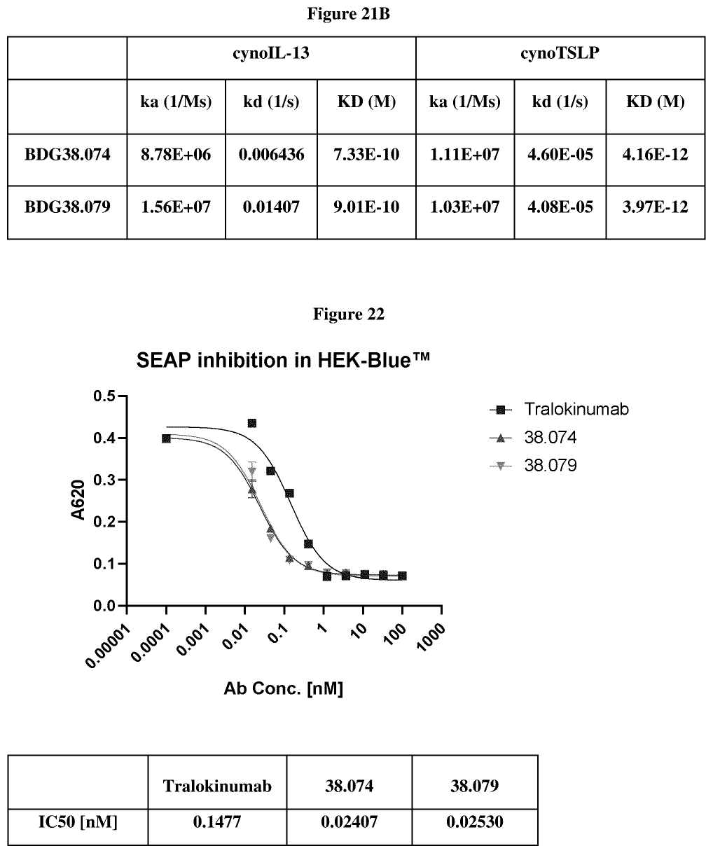

The results of SPR (Surface Plasmon Resonance) analysis of antibody BDG38.074 for human or cyno IL-13 or TSLP are shown in FIGS. 21A and 21B. Based on the SPR analysis, the affinities of BDG 38.074 to human and cynomolgus IL-13 are at double and triple digit picomolar, respectively, and the affinities to human and cynomolgus TSLP at a single digit picomolar.

FIG. 22A shows antibody BDG38.074 exhibits inhibition of CD23 expression similar to the anti-IL-13 benchmark (Tralokinumab). FIG. 22B shows antibody BDG38.074 inhibits TARC expression similar to anti-TSLP benchmark (Tezepelumab). These data demonstrate that while the anti-TSLP benchmark has only limited effect at inhibiting CD23 expression in monocytes, and the anti-IL-13 benchmark has only limited effect at inhibiting TARC levels, BDG38.074 inhibits both CD23 and TARC expression, demonstrating the unique ability of BDG dual antibodies to exert two distinct functions as a single standard IgG1 (LALA PG) antibody.

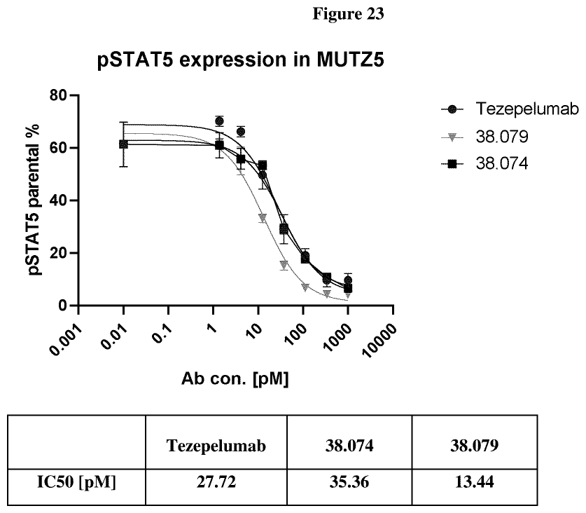

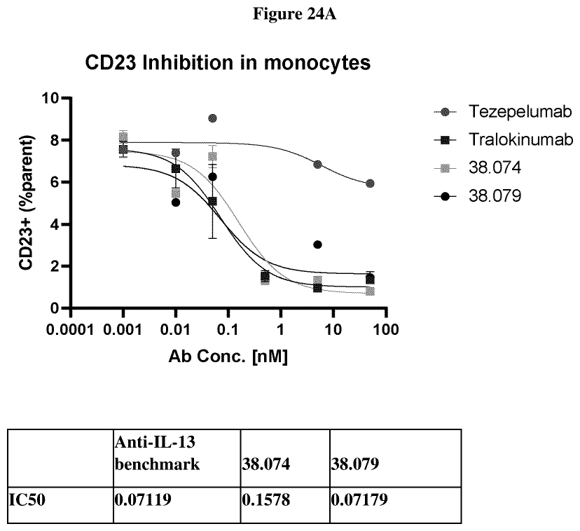

FIG. 23 shows antibody BDG38.074 exhibits functional inhibition similar to anti-TSLP benchmarks in MUTZ-5 cell line, demonstrating that BDG38.074 inhibits TSLP function with an IC50 of about 35 pM in cells expressing the native TSLP receptor subunits.

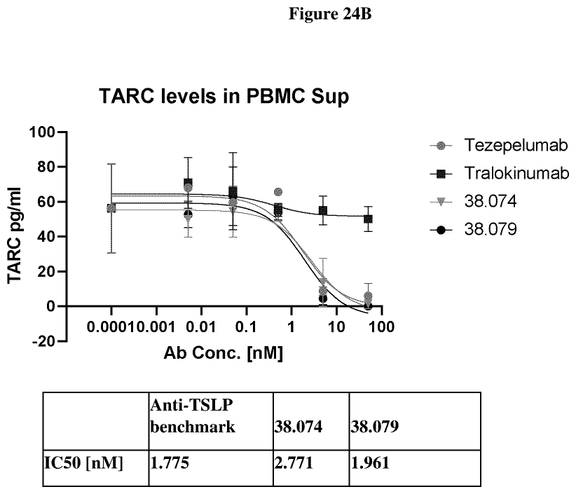

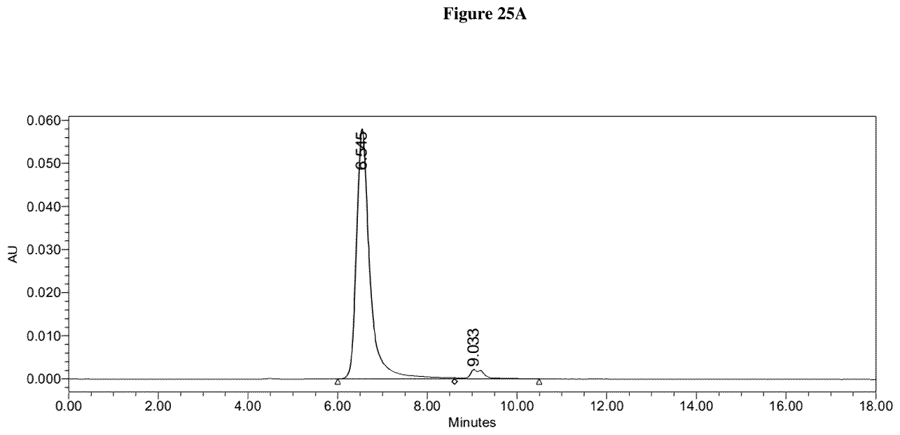

FIG. 24 shows antibody BDG38.074 inhibits IL-13 function in HEK reporter cell line with double digit picomolar affinity, demonstrating that BDG 38.074 inhibits IL-13 function in cells expressing the IL-13 receptor heterocomplex IL-4Ra and IL-13Rα1.

Antibody BDG38.079

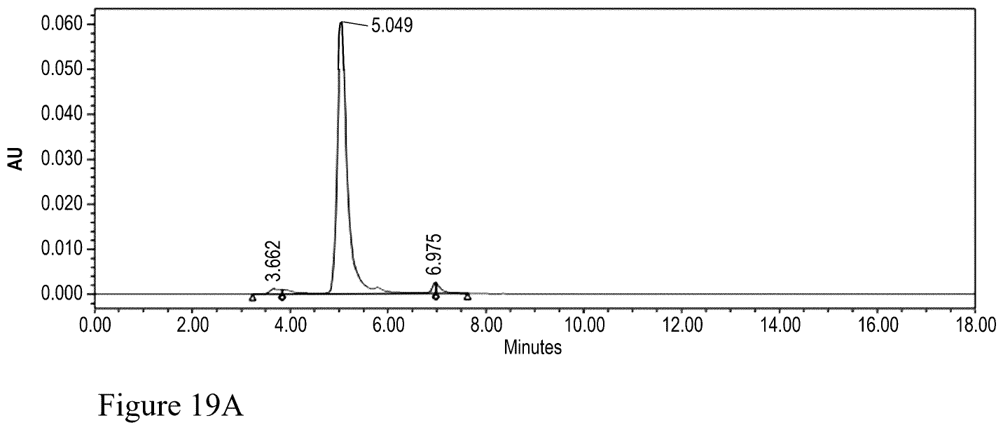

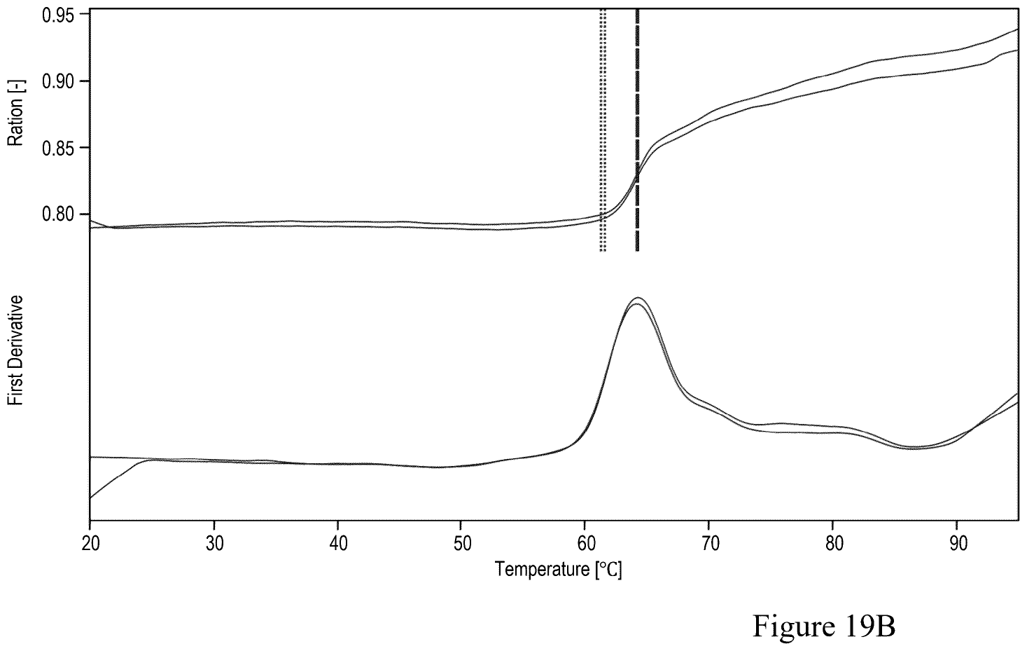

The size exclusion chromatography (SEC) scans and nano-differential scanning fluorimetry (DSF) analysis of the melting point for antibody BDG38.074 are shown in FIGS. 19A and 19B.

SEC analysis (FIG. 19A) was performed using BioResolve SEC mAb Column, 200 Å, 2.5 μm, 4.6×300 mm at 0.5 ml/min in PBS as a mobile phase and analyzed at 280 nm. BDG38.079 shows a predominant monodisperse peak with a minor percentage of aggregates (1.34%). DSF analysis (FIG. 19B) was performed using nanoDSF at a 1° C./min from 20-95° C. NanoDSF is monitoring the thermal unfolding of BDG38.079 according to the intrinsic fluorescence change at 350 and 330 nm. The top half of the graph shows the fluorescence ratio of 350 nm/330 nm as a function of temperature and the bottom half shows the first derivative as a function of temperature. BDG38.079 was analyzed at 0.5 mg/ml in PBS showed to have a T-onset of 61.4° C. and Tm of 64.2° C. suggesting a relatively stable fold.

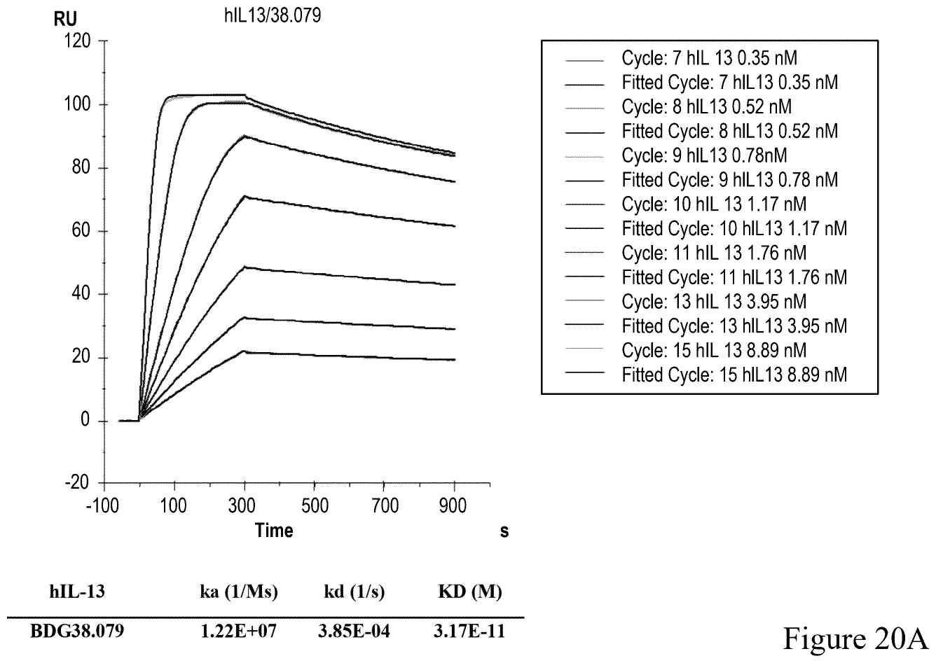

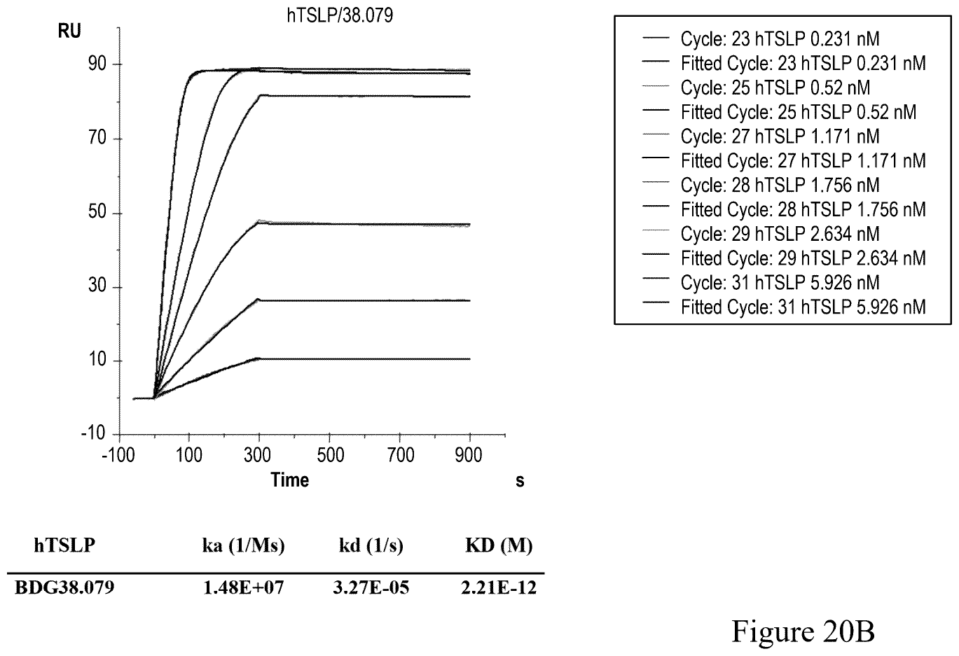

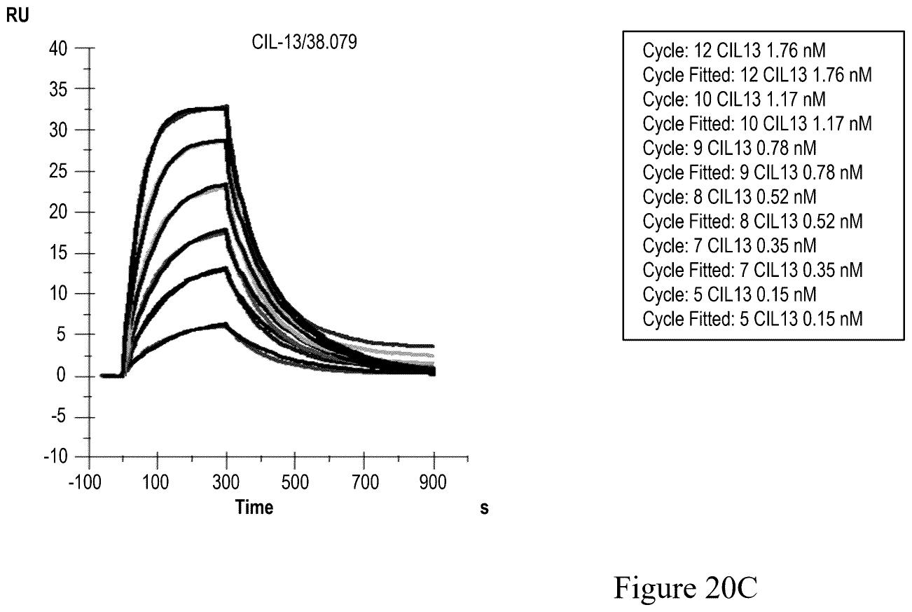

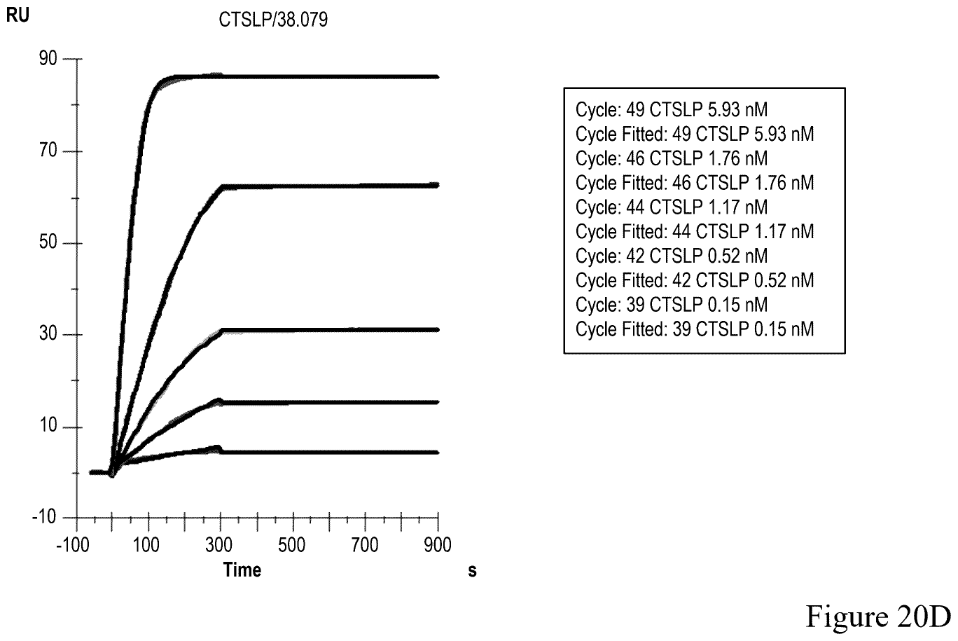

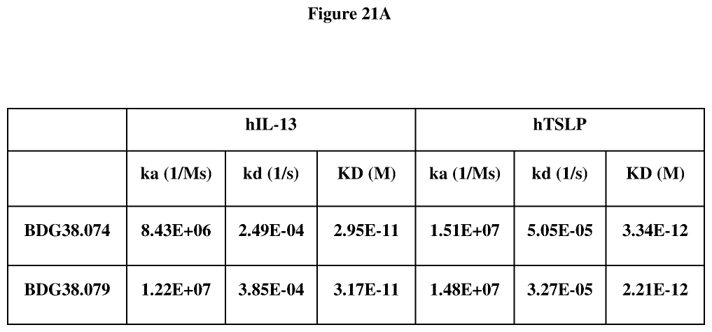

The binding affinities of antibody BDG38.079 to human/cyno IL-13 and human/cyno TSLP are shown in FIGS. 20A-20D. Surface Plasmon Resonance (SPR) analysis of BDG38.079 binding to human IL-13 (FIG. 20A), human TSLP (FIG. 20B), cyno IL-13 (FIG. 20C) and cyno TSLP (FIG. 20D) using BiacoreT200. CMS chip was coated with human antibody capture kit to obtain 3000-5000 RU and the antibody, served as the capture, was injected at a flow rate of 10 ul/ml to obtain 250-350 RU. Human/cynoTSLP and human/cynoIL-13 served as analytes in a concentration range of 30-0.153 nM. Contact time is 300 sec and dissociation time is 600 sec at a flow rate of 30 ul/min BDG38.079 exhibits high affinity binding to all cytokines tested, with KD values for hIL-13: 3.17E-11 M, for hTSLP: 2.21E-12 M, for cyno IL-: 13 9.01E-10 M and for cyno TSLP: 3.97E-12M.

Results of SPR (Surface Plasmon Resonance) analysis of antibody BDG38.079 for human or cyno IL-13 or TSLP are shown in FIG. 21. From the SPR analysis, the affinities of BDG 38.079 to human and cynomolgus IL-13 are double and triple digit picomolar respectively and the affinities to human and cynomolgus TSLP are single digit picomolar.

FIG. 22 shows antibody BDG38.079 inhibits IL-13 function in HEK reporter cell line with double digit picomolar affinity, demonstrating that BDG 38.079 inhibits IL-13 function in cells expressing the IL-13 receptor heterocomplex IL-4Ra and IL-13Rα1.

FIG. 23 shows antibody BDG38.079 exhibits similar functional inhibition to anti-TSLP benchmarks in MUTZ-5 cell line, demonstrating that BDG38.079 inhibits TSLP function with an IC50 of about 13 pM in cells expressing the native TSLP receptor subunits.

FIG. 24A shows antibody BDG38.079 exhibits similar inhibition of CD23 expression as the anti-IL-13 benchmark (Tralokinumab). FIG. 24B shows antibody BDG38.079 inhibits TARC expression similarly to anti-TSLP benchmark (Tezepelumab).

These data demonstrate that while the anti-TSLP benchmark has only limited effect at inhibiting CD23 expression in monocytes, and the anti-IL-13 benchmark has only limited effect at inhibiting TARC levels, BDG38.079 inhibits both CD23 and TARC expression, demonstrating the unique ability of BDG dual antibodies to exert two distinct functions as a single standard IgG1 (LALA PG) antibody.

Antibody BDG38.094

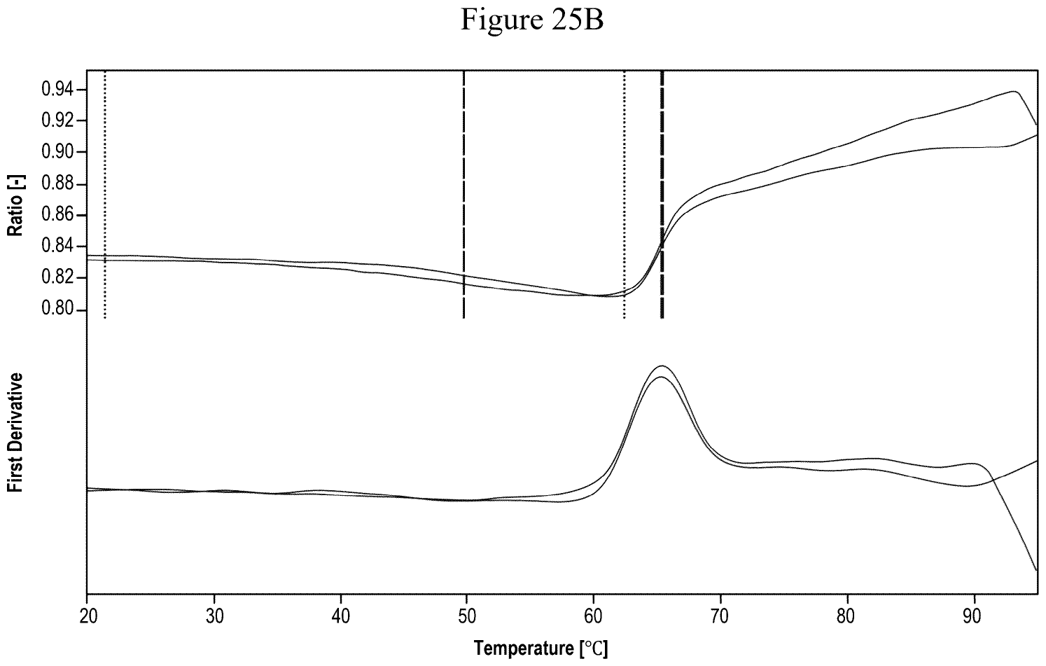

The size exclusion chromatography (SEC) scans and nano-differential scanning fluorimetry (DSF) analysis of the melting point for antibody BDG38.09 are shown in FIGS. 25A and 25B.

The size exclusion chromatography (SEC) scans and nano-differential scanning fluorimetry (DSF) analysis of the melting point for antibody BDG38.094 are shown in FIGS. 25A and 25B. SEC analysis (FIG. 25A) was performed using BioResolve SEC mAb Column, 200A, 2.5 μm, 4.6×300 mm at 0.4 ml/min in PBS as a mobile phase and analyzed at 280 nm. BDG38.094 shows a predominant monodisperse peak with undetectable aggregates. DSF analysis (FIG. 25B) was performed using nanoDSF at a 1° C./min from 20-95° C. NanoDSF is monitoring the thermal unfolding of BDG38.094 according to the intrinsic fluorescence change at 350 and 330 nm. The top half of the graph shows the fluorescence ratio of 350 nm/330 nm as a function of temperature and the bottom half shows the first derivative as a function of temperature. BDG38.094 was analyzed at 0.5 mg/ml in PBS showed to have a T-onset of 62.4° C. and Tm of 65.3° C. suggesting a relatively stable fold.

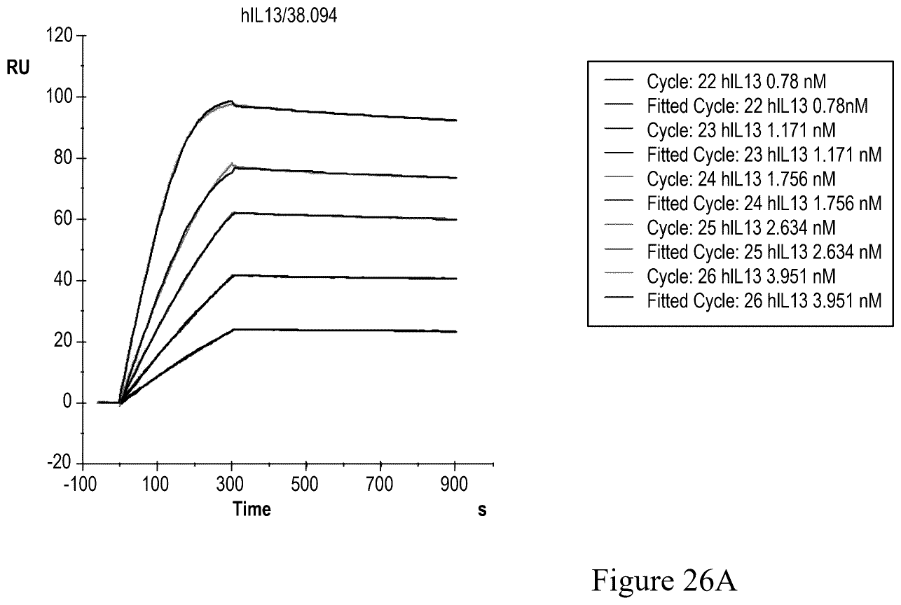

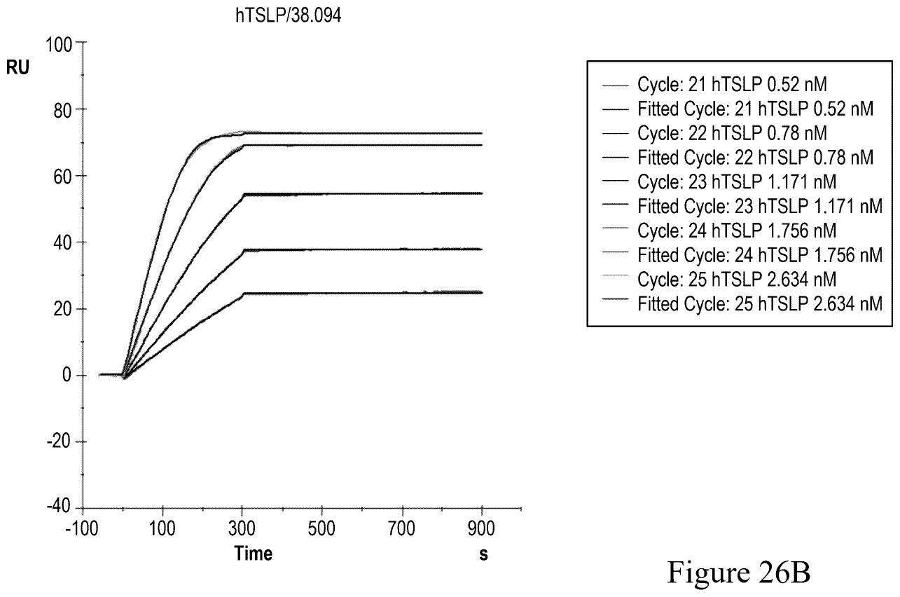

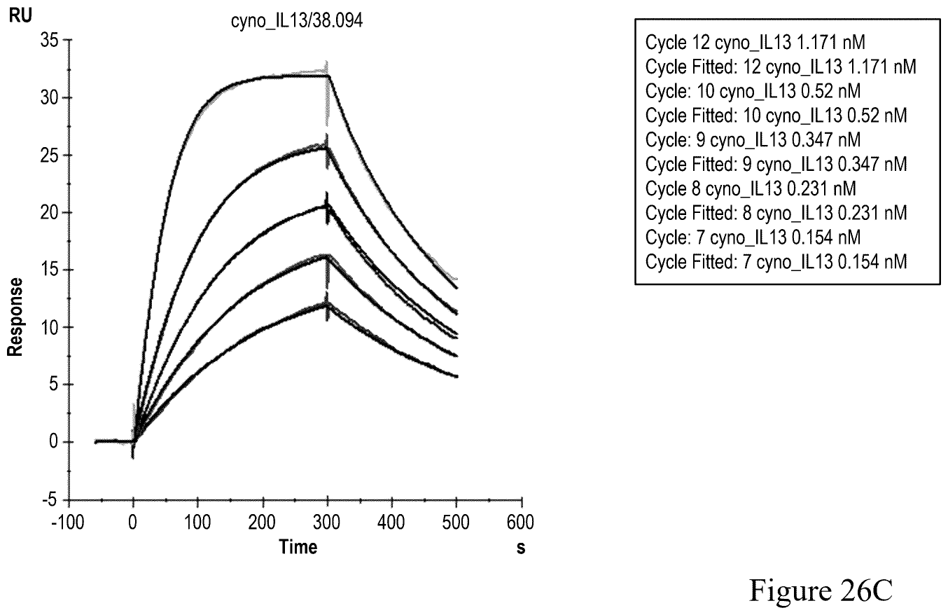

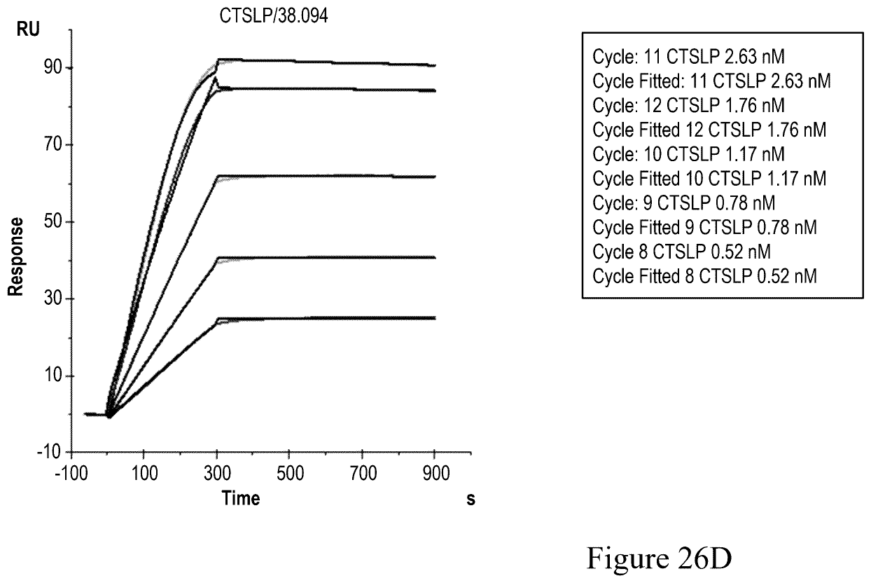

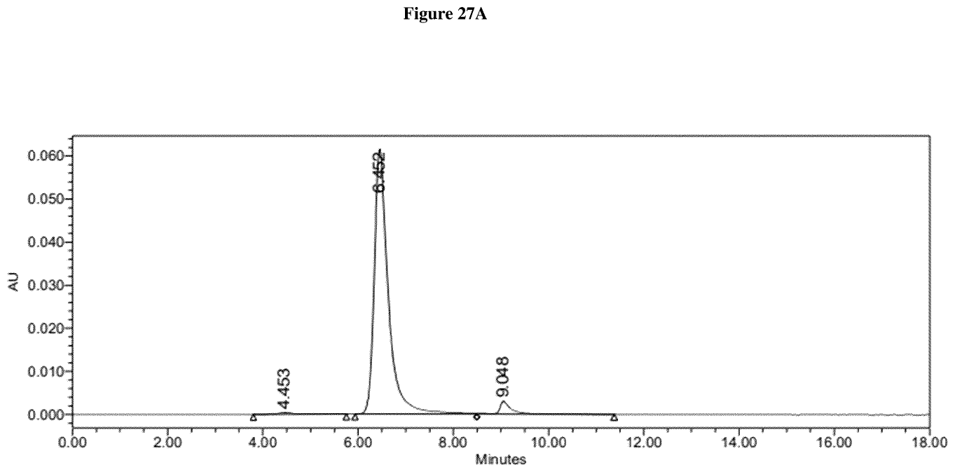

The binding affinities of antibody BDG38.094 to human/cyno IL-13 and human/cyno TSLP are shown in FIGS. 26A-26D. Surface Plasmon Resonance (SPR) analysis of BDG38.094 binding to human IL-13 (FIG. 26A), human TSLP (FIG. 26B), cyno IL-13 (FIG. 26C) and cyno TSLP (FIG. 26D) using BiacoreT200. CMS chip was coated with human antibody capture kit to obtain 3000-5000 RU and the antibody, served as the capture, was injected at a flow rate of 10 ul/ml to obtain 250-350 RU. Human/cyno TSLP and human/cyno IL-13 served as analytes in a concentration range of 30-0.153 nM. Contact time was 300 sec and dissociation time was 600 sec (for cyno IL-13 dissociation time was 200 sec)) at a flow rate of 30 ul/min BDG38.094 shows a high affinity to all cytokines with KD values for hIL-13: 1.38E-11 M for hTSLP: <1E-12 M (below limit of detection), for cyno IL-13: 2.70E-10 M and for cyno TSLP: 2.75E-12 M.

Antibody BDG38.138

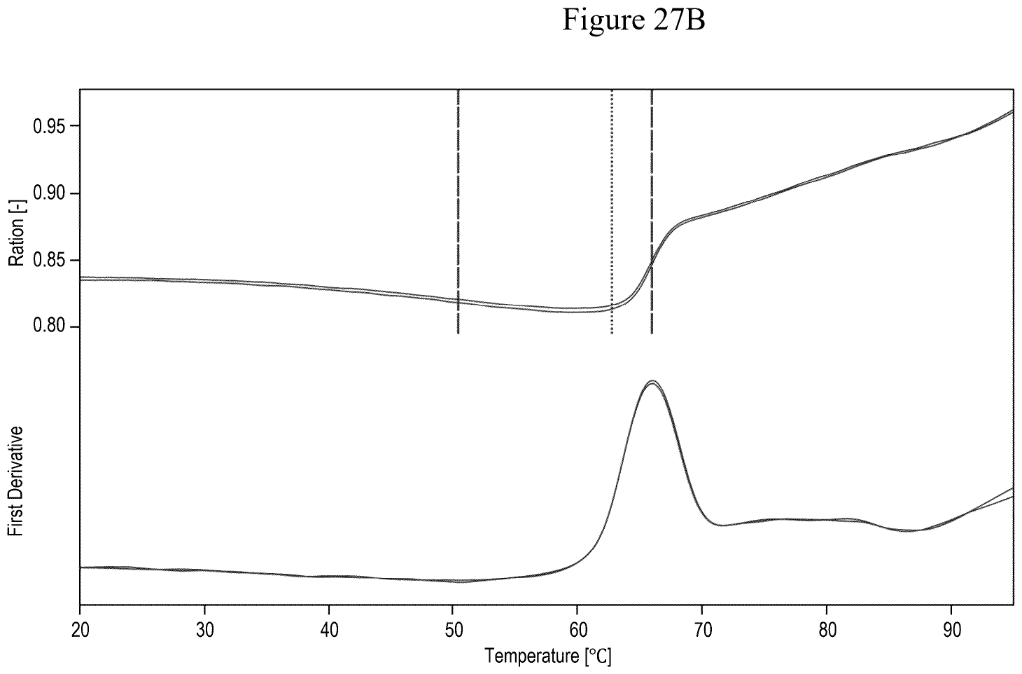

The size exclusion chromatography (SEC) scans and nano-differential scanning fluorimetry (DSF) analysis of the melting point for antibody BDG38.138 are shown in FIGS. 27A and 27B.

SEC was performed using BioResolve SEC mAb Column, 200 Å, 2.5 μm, 4.6×300 mm at 0.4 ml/min in PBS as a mobile phase and analyzed at 280 nm. BDG38.138 shows a predominant monodisperse peak with 0.8% aggregates. DSF analysis was performed using nanoDSF at a 1° C./min from 20-95° C. NanoDSF is monitoring the thermal unfolding of BDG38.138 according to the intrinsic fluorescence change at 350 and 330 nm. The top half of the graph shows the fluorescence ratio of 350 nm/330 nm as a function of temperature and the bottom half shows the first derivative as a function of temperature. BDG38.138 was analyzed at 0.5 mg/ml in PBS showed to have a T-onset of 62.8° C. and Tm of 66° C. suggesting a relatively stable fold.

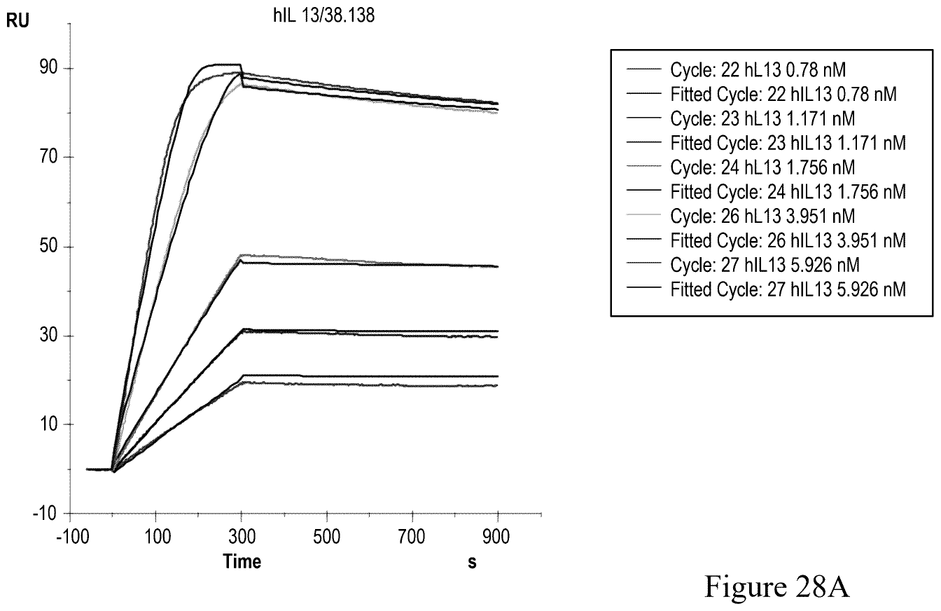

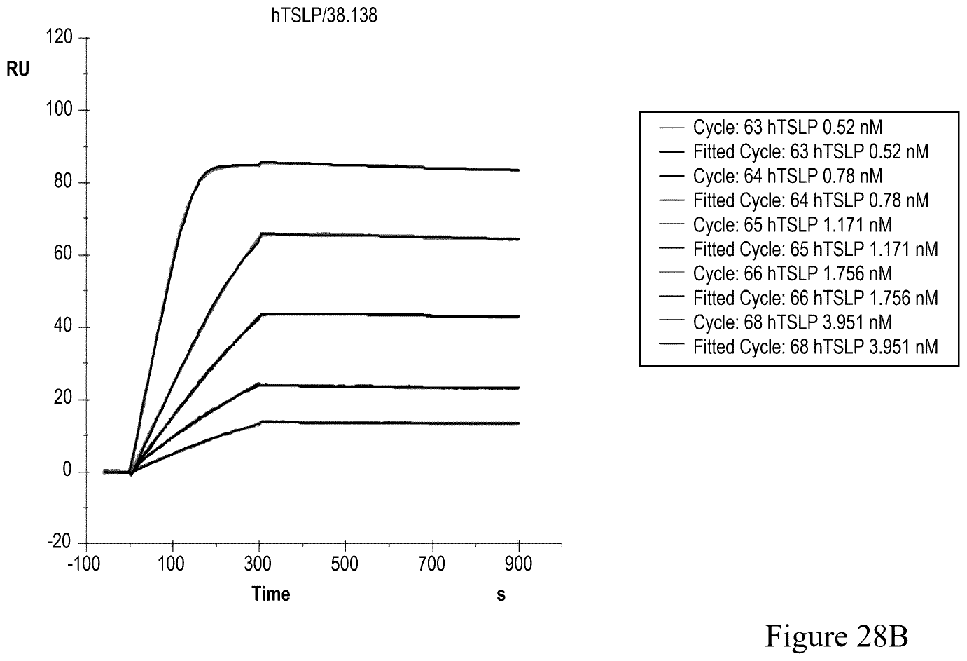

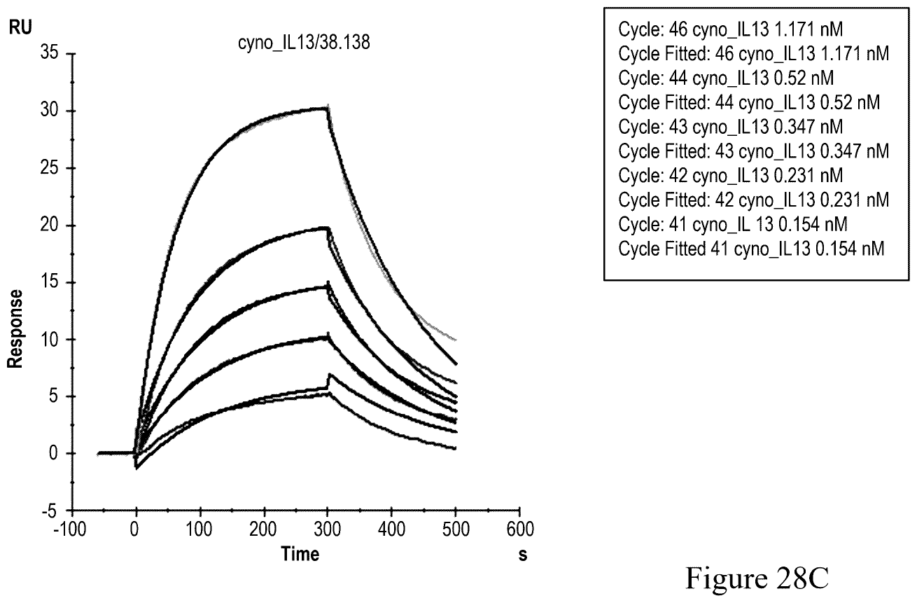

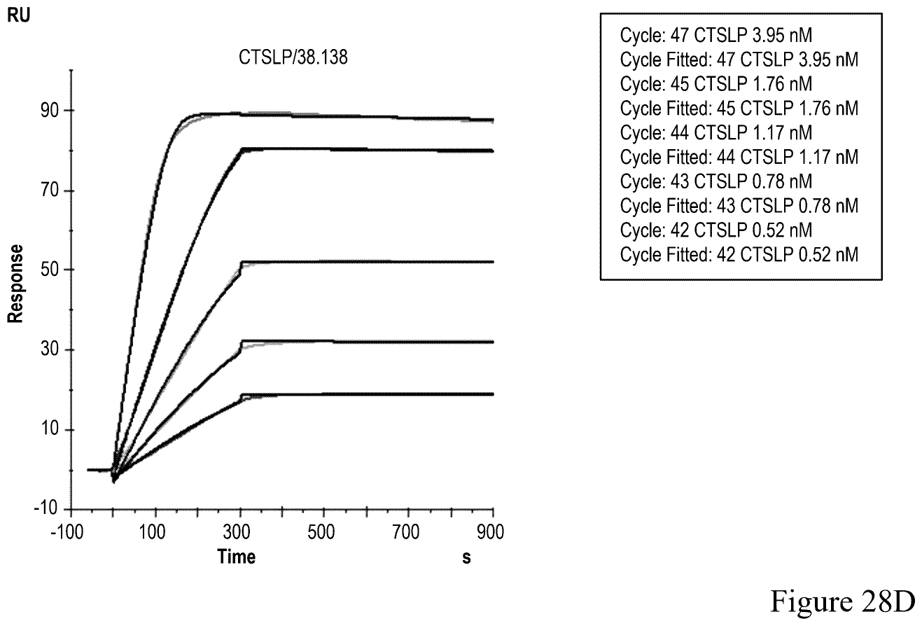

The binding affinities of antibody BDG38.138 to human/cyno IL-13 and human/cyno TSLP are shown in FIGS. 28A-28D. (Surface Plasmon Resonance (SPR) analysis of BDG38.138 binding to human IL-13 (FIG. 28A), human TSLP (FIG. 28B), cyno IL-13 (FIG. 28C) and cyno TSLP (FIG. 28D) using BiacoreT200. CMS chip was coated with human antibody capture kit to obtain 3000-5000 RU and the antibody, served as the capture, was injected at a flow rate of 10 ul/ml to obtain 250-350 RU. Human/cyno TSLP and h/cyno IL-13 served as analytes in a concentration range of 30-0.153 nM. Contact time was 300 sec and dissociation time was 600 sec (for cyno IL-13 dissociation time was 200 sec) at a flow rate of 30 ul/min BDG38.138 shows a high affinity to all cytokines with KD values for hIL-13: 1.34E-11 M, for hTSLP: 2.85E-12 M, for cynoIL-13: 8.49E-10 M and for cyno TSLP: 1.35E-12 M.

### SUMMARY

The presented data demonstrate that BDG dual antibodies are highly monomeric and stable molecules (Table 11). Those antibodies can bind human IL-13, cynomolgus IL-13, human TSLP and cynomolgus TSLP with picomolar affinities (FIGS. 16A-16F). Unlike different bi-specific antibodies, each BDG dual antibody mentioned in the patent is a standard IgG1 that can bind the above mentioned antigens (IL-13 and TSLP). In the case of antibody clone BDG38.138 it is a standard IgG1 with the Fc mutations as described herein. (LALA, PG) that can bind the above-mentioned antigens.

The full length heavy chain (HC) for BDG38.138
is set forth in SEQ ID NO: 408:
QMQLVESGGGVVQPGRSLRLSCAASGFAFRTYGMHWVRQAPGKGLEW
VAVIWDDGSNTHYADSVKGRFTITRDNSKNTLNLQMNSLRAEDTAVY
YCVRAPQWYLTAEAFDLWGQGTMVTVSSASTKGPSVFPLAPSSKSTS
GGTAALGCLVKDYFPEPVTVSWNSGALTSGVHTFPAVLQSSGLYSLS
SVVTVPSSSLGTQTYICNVNHKPSNTKVDKKVEPKSCDKTHTCPPCP
APEAAGGPSVFLFPPKPKDTLMISRTPEVTCVVVDVSHEDPEVKFNW
YVDGVEVHNAKTKPREEQYNSTYRVVSVLTVLHQDWLNGKEYKCKVS
NKALGAPIEKTISKAKGQPREPQVYTLPPSRDELTKNQVSLTCLVKG
FYPSDIAVEWESNGQPENNYKTTPPVLDSDGSFFLYSKLTVDKSRWQ
QGNVFSCSVMHEALHNHYTQKSLSLSPGK.
The full length light chain (LC) for BDG38.138
is set forth in SEQ ID NO: 409:
SYVLTQPPSVSVAPGETASITCGGNMIGGYLVHWYQQKPGQAPVLVI
YDDVDRPSRIPERFSGSNSGNTATLTISRVEAGDEADYYCQVWDHNS
NHMVFGGGTKLTVLGQPKAAPSVTLFPPSSEELQANKATLVCLISDF
YPGAVTVAWKADSSPVKAGVETTTPSKQSNNKYAASSYLSLTPEQWK
SHRSYSCQVTHEGSTVEKTVAPTECS.

Since some of the antibody's binding was above the limit of detection IC50 competition ELISA against Tezepelumab was done (Table 13, FIGS. 14A-14C). This data supports the SPR analysis and demonstrates that BDG dual antibodies can compete with Tezepelumab over binding to TSLP.

BDG antibodies demonstrated functional inhibition of IL-13 signaling in HEK reporter cells, by blocking signal transduction of IL-13 through STATE (Table 15, FIG. 22). Similarly, BDG antibodies inhibited TSLP mediated STATS phosphorylation in MUTZ cells, expressing the natural TSLP-receptor heterocomplex comprising TSLP-R and IL-7Rα.

To test the effect of a dual antibody, hPBMC was treated with both hTSLP and hIL-13 for 48 hours in the presence of rising concentration of BDG antibodies or benchmark antibodies (Table 12, FIGS. 24A and 24B). In this setup TSLP treatment led to increase in TARC level and 11-13 treatment led to increase in CD23 levels. While BDG dual antibodies inhibited CD23 expression similarly to the anti-IL-13 benchmark (Tralokinumab), anti-TSLP benchmark had only minor effect. Similarly, while BDG dual antibodies inhibited TARC secretion similarly to Tezepelumab, anti-IL-13 benchmark had only minor effect on the level of TARC. This demonstrates that BDG dual antibodies inhibit as a single IgG1 molecule having both functions of TSLP and IL-13.

### Example 6: Biochemical Comparison of Antibodies Having the Same VH/VL Regions with Different Fc Formats (hIgG1 LALA (BDG38.145) Vs LALAPG (BDG38.138))

Objective: To examine the biochemical properties of hIgG1 antibodies with different Fc formats, wherein the antibodies comprise the CDR regions as follows: HCDR1 is SEQ ID NO: 349, HCDR2 is SEQ ID NO: 350, HCDR3 is SEQ ID NO: 351, LCDR1 is SEQ ID NO: 364, LCDR2 is amino acids sequence DDV, LCDR3 is 384, and comprise the VH/VL regions as follows: VH is SEQ ID NO: 337 and VL is SEQ ID NO: 338. Specifically, two hIgG1 antibodies comprising the identical CDR and VH/VL regions with those of clone 38.138 were compared, wherein the Fc regions differed so that the effects of the 2 Fc formats could be compared.

The formats for comparison were 38.138 (hIgG1 LALA-PG) and 38.145 (hIgG1 LALA). BDG38.138 and BSG38.145 share the same VH and VL sequences, and only differ in the Fc mutations. As used throughout, the names “BDG38.138” and “38.138” have the same meanings and indicate the same antibody, similarly, the names “BDG38.145” and “38.145” have the same meanings and indicate the same antibody.

The full length heavy chain (HC) amino acid sequence is provided for BDG38.138 in Example 5. The full length heavy chain (HC) amino acid sequence for BDG38.145 is set forth in SEQ ID NO: 410: QMQLVESGGGVVQPGRSLRLSCAASGFAFRTYGMHWVRQAPGKGLEWVAVIWDDG SNTHYADSVKGRFTITRDNSKNTLNLQMNSLRAEDTAVYYCVRAPQWYLTAEAFDL WGQGTMVTVSSASTKGPSVFPLAPSSKSTSGGTAALGCLVKDYFPEPVTVSWNSGALT SGVHTFPAVLQSSGLYSLSSVVTVPSSSLGTQTYICNVNHKPSNTKVDKKVEPKSCDKT HTCPPCPAPEAAGGPSVFLFPPKPKDTLMISRTPEVTCVVVDVSHEDPEVKFNWYVDG VEVHNAKTKPREEQYNSTYRVVSVLTVLHQDWLNGKEYKCKVSNKALPAPIEKTISK AKGQPREPQVYTLPPSRDELTKNQVSLTCLVKGFYPSDIAVEWESNGQPENNYKTTPP VLDSDGSFFLYSKLTVDKSRWQQGNVFSCSVMHEALHNHYTQKSLSLSPGK (SEQ ID NO: 410). The full length light chain (LC) for both BDG38.138 and BDG38.145 is set forth in SEQ ID NO: 109, as presented in Example 5.

Methods

Surface Plasmon Resonance (SPR)—Kinetic measurements of 38.138 and 38.145 for hIL-13 and hTSLP were done using Biacore 5200 [Cytiva, USA] on CMS chip [Cytiva, br-10005-30]. The chip was crosslinked with a human antibody capture kit [Cytiva, USA br-100839] according to manufacturer's protocol to target 5000-8000 RU.

In order to measure kinetics, a multi-cycle strategy was used. Antibodies were captured on the coated chip to reach ˜200 RU. Human/CynolL-13 [Acro Biosystems, IL3-H52H4/IL3-C5249] or human/cynoTSLP [Acro Biosystems, TSP-H52H4/TSP-052H8] were injected from 60 nM to 0.3 nM in 1.5 dilutions in a contact time of 300 sec, at flow rate of 30 ul/min Dissociation was done by injecting PBS-T for 400 sec at a flow rate of 30 ul/min Between each cycle, all channels underwent regeneration using 3M MgCl₂. Binding kinetics were determined by the 1:1 Binding model using the Biacore 5200 evaluation software, version 1.1.

Stability at viral inactivation conditions: A total of 4.4 mg/ml of 38.138 and 38.145 were divided into 2.2 mg/ml that was used as a control and 2.2 mg/ml that was incubated at a viral inactivation condition. All samples were buffer exchanged from PBS, pH 7.4 into either Na-acetate, pH 3.5 (treated) or PBS, pH 7.4 (control) using PD10 desalting column [Cytiva, USA 17085101]. Samples were incubated for 1 hour at room temperature followed by a 2n d buffer exchange into PBS, pH 7.4. antibodies concentration and volume were monitored after each buffer exchange and incubation step using 280 nm. At the end of the viral inactivation process, all samples (treated and control) were analyzed using SDS-PAGE R/NR, SEC, ELISA EC50 and HIC.

Size exclusion chromatography (SEC)—SEC analysis was done by loading 12 μg from each viral inactivation sample on BioResolve SEC mAb 200A 2.5 um 4.6×300 mm column (Waters Corporation MA, USA) that was preequilibrated with PBS, pH 7.4 at a flow rate of 0.4 ml/min, room temperature. Chromatogram analysis was obtained at 280 nm using an ACQUITY Arc HPLC system (Waters Corporation MA, USA).

SDS-PAGE under reduced and non-reducing conditions—For SDS-PAGE analysis, samples were prepared by mixing viral inactivation and control samples with lithium dodecyl sulfate (LDS) [GenScript, USA M00676] with or without 2-mercaptoethanol for reducing and non-reducing conditions, respectively. Samples were boiled at 80° C. for 5 min, and a total of 2.5 pg was loaded on a gradient 4-20% SDS-PAGE [Invitrogen, MA USA; XP04125BOX].

Hydrophobic interaction chromatography (HIC)— HIC analysis was done by spiking 7.5 μg from each viral inactivation sample into buffer A (2M ammonium sulphate, 0.1M sodium phosphate) to achieve a final ammonium sulphate concentration of 1M. A Proteomix HIC Butyl-NP5 5 μm Non-Porous 4.6×35 mm column (Sepax, USA) was used with a linear gradient from 90% A and 10% B buffer (0.1M sodium phosphate, pH 6.5) to 100% B buffer over 20 min at a flow rate of 1 ml/min, at 30° C. Chromatogram analysis was obtained at 280 nm using the ACQUITY Arc HPLC system (Waters Corporation MA, USA).

ELISA EC50—EC50 ELISA was performed to evaluate affinity integrity of the variants post viral inactivation process. A high binding 96 wells plate was coated with 50 ng/well of either hIL-13 or hTSLP and incubated over night at 4° C. Following coating, plates were washed 3 times using PBS-T and blocking solution containing 1.5% BSA in PBS-T was applied over plates for 1 hour incubation at room temperature. After a 2n d washing step, 2-fold dilution of each viral inactivation or control sample from 70 nM to 0.068 nM were added to well in the plates and incubated for 1 hour at room temperature. A 3^{rd} step of washing was performed and goat anti human Fc-HRP (Jackson, USA; 109-035-008) was applied over the plates and incubated for 30 min at room temperature. Development of plates was done after a 4^{th} washing step using 3,3′,5,5′-Tetramethylbenzidine (TMB) (SURMODICS, USA; TMBW-0100-01) and reaction was stopped using stop solution [SURMODICS, USA; LSTP-0100-01]. Signals were recorded at 450 nm and analysis was done using GraphPad Prism software (GraphPad Software, LLC), using “[Agonist] vs. response—Variable slope (four parameters)” analysis

Differential Scanning Fluorimetry (DSF) measurements—To compare the thermal stability of 38.138 and 38.145, Differential Scanning Fluorimetry was performed using a NanoDSF Prometheus NT48 instrument assessing the thermal transition from the native to unfolded state. Antibodies at 0.5 mg/ml concentration in PBS pH 7.4 were loaded and DSF was performed at a temperature increment of 1° C./min.

PBMCs activation and treatment—Peripheral blood mononuclear cells (PBMCs) (Cell Generation, Israel; CAT: 10102510) were used to determine hIL-13 and hTLSP inhibition. PBMCs were thawed and cultured in growth medium comprising of RPMI—1640, 10% FBS, 1% Glutamax, 1% Sodium-Pyruvate, 0.1% 2-ME, 1% Pen-Strep and 1% nonessential AA. The cells were seeded in 96 well plate at 5×10⁵ cells/well and were allowed to rest for 1 hour at 37° C., 5% CO₂. hIL-13-His (Acro Biosystems, USA; CAT: IL3-H52H4) at a concentration of 0.7 nM and hTSLP-His (Acro Biosystems, USA; CAT: TSP-H52Hb) at a concentration of 70 pM were added to the cells, followed immediately by addition of antibodies in serial dilutions, to a total of to a total of 200 uL/well. Cells were incubated for 48 hours at 37° C., 5% CO₂.

CD23 expression in monocytes—IC₅₀ of antibody inhibition of hIL-13 was determined by measuring CD23 expression level in monocytes. At the end of 48 hours incubation of the cells with different concentrations of antibodies, monocytes were detached from the bottom of the wells using cold PBS and scraping. Cells were labeled using PE-CF594-conjugated anti-CD3 (BD Bioscience, USA; CAT: 562280), PE/Cyanine7-conjugated anti-CD14 (BioLegend, USA; CAT: 301814), APC-conjugated anti-CD19 (Bio Legend, USA; CAT: 302212) and FITC-conjugated anti-CD23 (BioLegend, USA; CAT: 338506) antibodies. CD23 percentage of CD14+ population was measured using CytoFLEX flow cytometer (Beckman Coulter, USA).

TARC levels in PBMCs supernatant—TARC levels were determined using TARC DUOSET ELISA kit (R&D systems, USA; CAT: DY364) according to the manufacturer's instructions. Briefly, ELISA high binding protein plates were coated with capture antibody (antibodies being assessed), diluted in PBS. Plates were sealed and incubated overnight at 4° C. The following day plates were washed and blocked using 1% BSA in PBS at room temperature, with shaking for 1 hour. The plates were washed three times in phosphate-buffered saline with Tween 20 (PBST), and supernatant from the PBMCs plates was diluted 1:1 in 1% BSA in PBS, transferred to the wells and incubated at room temperature with shaking for 2 hours. The plates were washed three times in PBST. Detection was performed using the kit's detection antibody (biotinylated) in PBS 1% BSA at room temperature with shaking for 2 hours followed by three washes in PBST and addition of Streptavidin—HRP in PBS 1% BSA for 30 minutes. Following 3 washes in PBST, the plates were developed by adding TMB and TMB-stop solution, and 450 nm. Values were analyzed using standard sample curve.

Results:

Specifications of Fc mutations:

Clone 38.138, human IgG1 with the Fc-modification LALA-PG (substituting alanine for leucine at positions 234 and 235 and substituting proline for glycine at position 329 of the Fc region).

Clone 38.145, human IgG1 with the Fc-modification LALA (substituting alanine for leucine at positions 234 and 235).

Clones 38.138 and 38.145 were compared in a series of biochemical assays: Surface Plasmon Resonance (SPR) analyses for human IL-13 (hIL-13) and human TSLP (hTSLP) in repetitions show similar kinetics for both formats. SPR was performed by capturing the antibody (38.138 or 38.145) onto an anti-hFc precoated CMS chip, and a serial dilution of IL-13/TSLP was injected in a multi-cycle strategy.

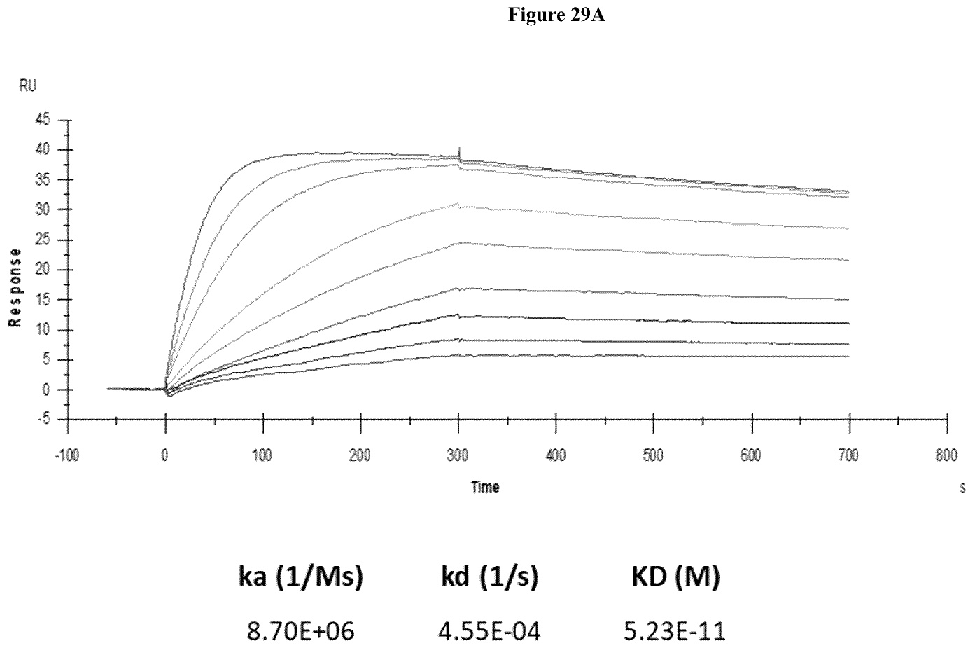

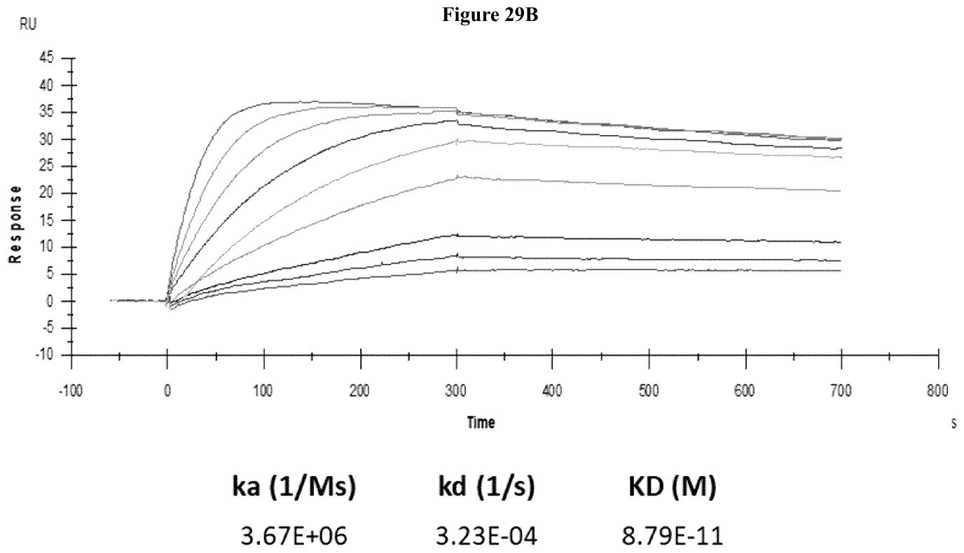

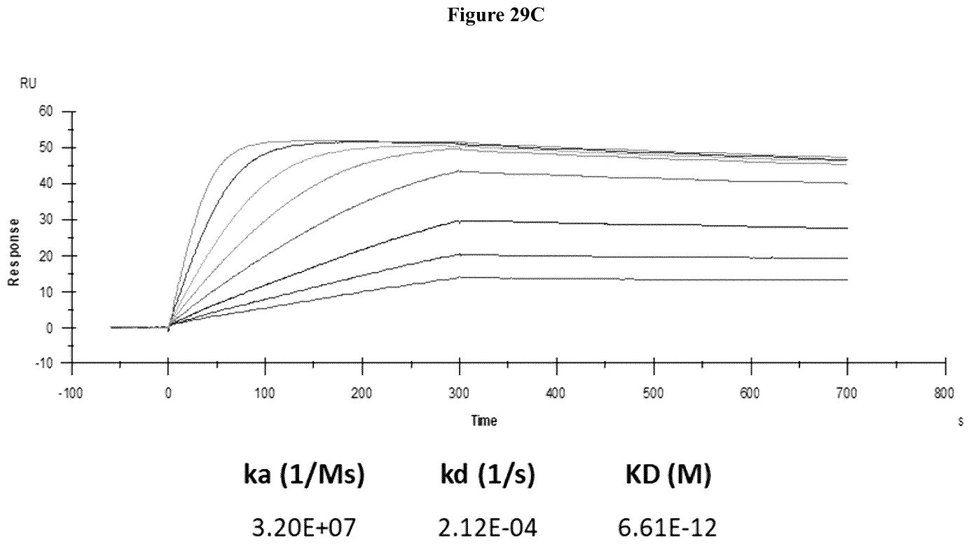

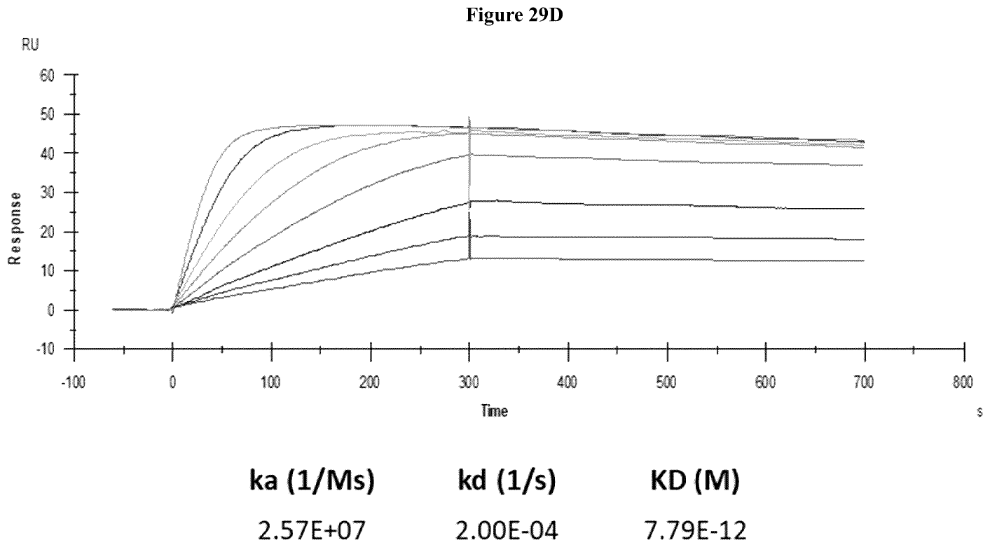

FIGS. 29A-29D show the results of SPR (Surface Plasmon Resonance) analysis for the binding of hIgG1-LALAPG (38.138) or hIgG1-LALA (38.145) to human IL-13 and human TSLP. The antibodies comprising either the Fc LALA mutations or the Fc LALAPG mutations demonstrated similar affinities to both hIL-13 and hTSLP.

The stability of hIgG1-LALAPG (38.138) or hIgG1-LALA (38.145) under viral inactivation conditions was tested. Viral inactivation is a necessary process in biotherapeutic molecule production which is designed to enhance the safety of products that may contain or can become contaminated with viruses during production or processing. For immunoglobulin mAb products, low pH is the most frequently used method for viral inactivation; the common process includes incubation of the Ab in a low pH buffer for 1 hour at room temperature.

Stability was assessed by concentration measurement, SDS-PAGE, SEC, HIC, and binding following the viral inactivation process. Clones 38.138 (hIgG1 LALAPG) and 38.145 (hIgG1 LALA) were incubated for 1 hour with sodium acetate (NaAc) pH 3.5 at room temperature. Concentrations were monitored throughout the buffer exchange/inactivation process.

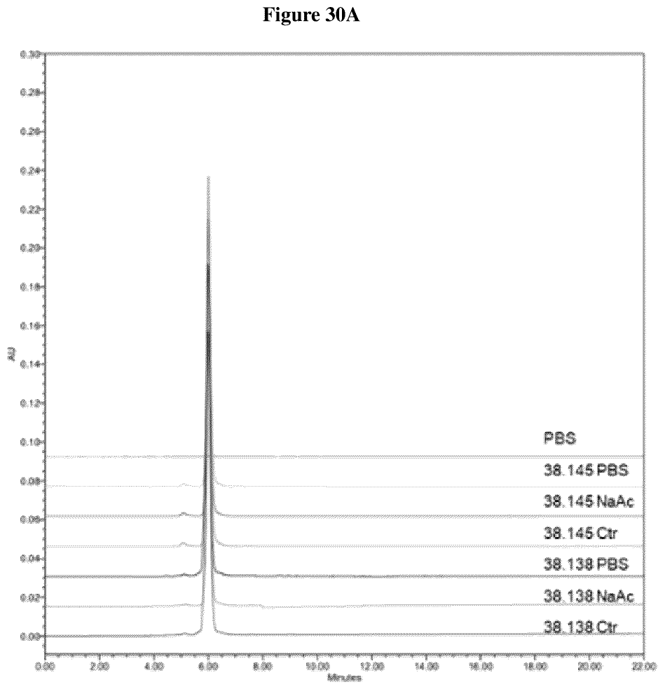

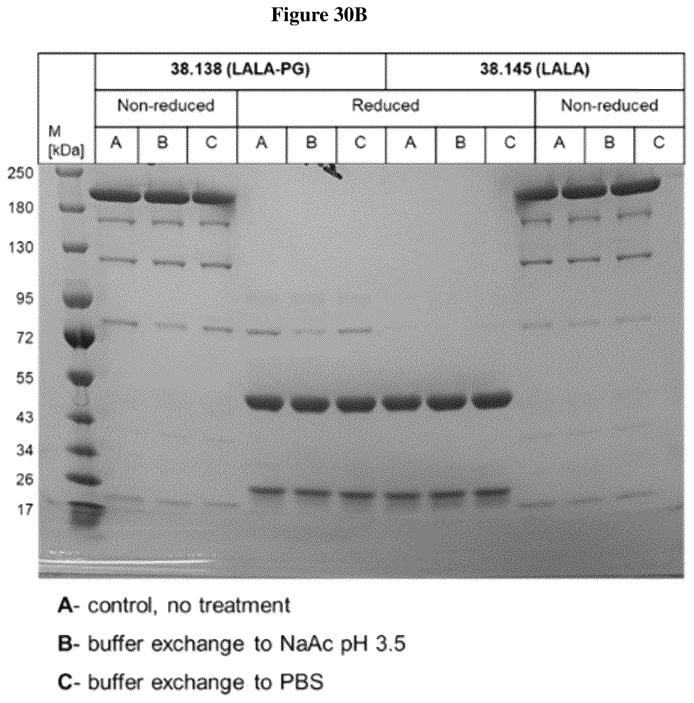

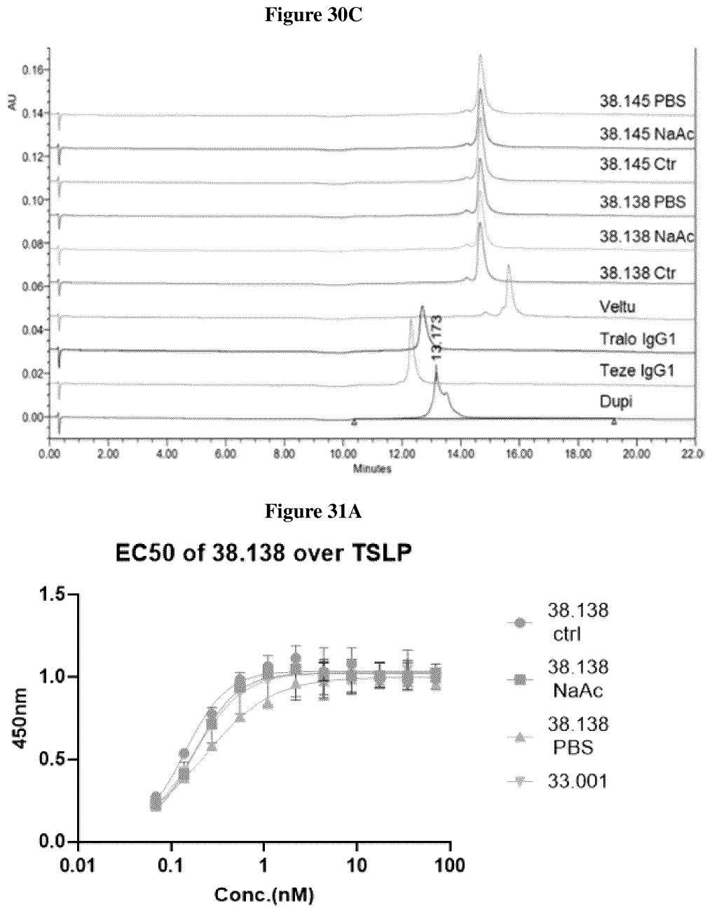

FIGS. 30A-30B show the biochemical properties of 38.138 (hIgG1 LALAPG) and 38.145 (hIgG1 LALA) following incubation at viral inactivation conditions. Samples were buffer exchanged from PBS, pH 7.4 into either Na-acetate, pH 3.5 (treated) or PBS, pH 7.4 (control), then incubated for 1 hour at room temperature followed by a 2nd buffer exchange into PBS, pH 7.4. Antibody concentration and volume were monitored after each buffer exchange and incubation step to evaluate if any material was lost in the process. At the end of the viral inactivation process, all samples (treated and control) were analyzed using SDS-PAGE, size exclusion chromatography (SEC), and Hydrophobic interaction chromatography (HIC). The binding of the treated antibodies to hIL-13 and hTSLP was measured by ELISA. The dual binding anti-IL-13/TSLP antibodies in IgG1 LALAPG and IgG1 LALA format showed similar biochemical stability under viral inactivation conditions.

To determine ELISA EC50 measurements, wells were coated with the respective cytokine, then incubated with clones 38.138 or 38.145 after incubation under viral inactivation conditions (NaAc) or control conditions (non-treated antibodies). Control antibodies went through the same steps of the viral inactivation process but in PBS instead of Sodium Acetate (NaAc) at a concentration range of 0.06 nM to 70 nM. Further, wells were washed and developed using HRP conjugated secondary antibody.

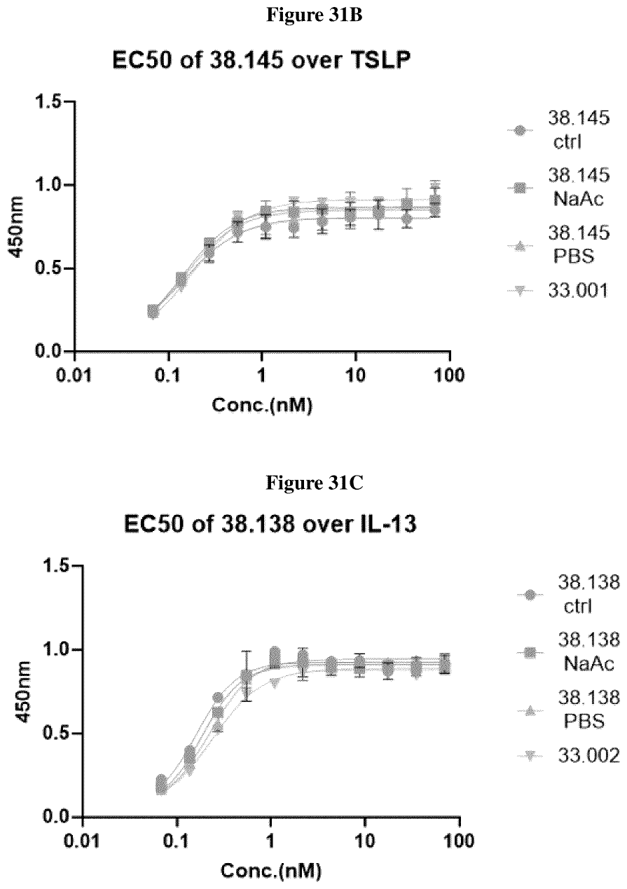

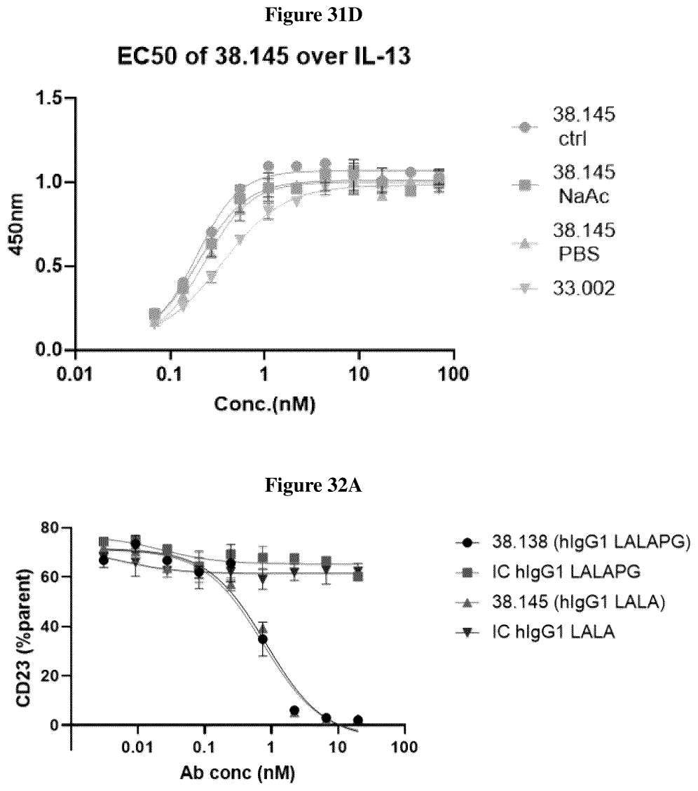

FIGS. 31A-31C present ELISA EC50 binding of 38.138 (hIgG1 LALAPG) and 38.145 (hIgG1 LALA) antibodies that had been treated with NaAc or PBS (control).i.e., viral inactivation conditions, to human TSLP and human IL-13. Anti-TSLP antibody 33.001 (Tezepelumab IgG1) and anti-IL-13 antibody 33.02 (Tralokinumab IgG1) were used as positive controls.

Additionally, Clone 38.138 and Clone 38.145 having the two Fc formats were compared for functional activity in human PBMCs, testing the CD23 expression in monocytes and TARC levels.

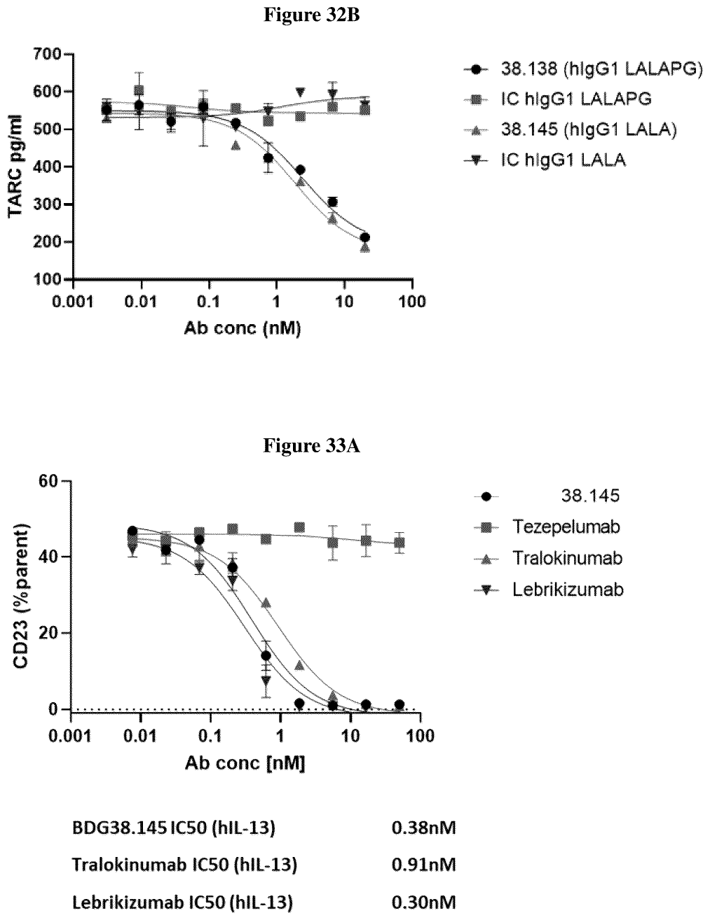

FIG. 32A shows antibodies 38.138 (hIgG1 LALAPG) and 38.145 (hIgG LALA) exhibit similar inhibition of CD23 expression in hPBMCs. IC50 of antibody inhibition of IL-13 was determined by measuring CD23 expression level in monocytes. At the end of 48 hours incubation of the cells with different concentrations of antibodies, the cells were detached from the bottom of the wells and labeled using PE-CF594-conjugated anti-CD3, PE-Cy7-conjugated anti-CD14, APC-conjugated anti-CD19 and FITC-conjugated anti-CD23 antibodies. CD23 percentage of CD14+ population was measured using flow cytometry. FIG. 32B shows antibodies 38.138 (hIgG1 LALAPG) and 38.145 (hIgG LALA) inhibit TARC expression in a similar manner IC50 of antibody inhibition of hTSLP and hIL-13 was determined by TARC inhibition as measured by ELISA.

Summary: The biochemical comparison performed here demonstrates that antibodies 38.138 (hIgG1 LALAPG) and 38.145 (hIgG LALA) exhibit similar functionality.

### Example 7: Functional Comparison of Antibodies Having the Same VH/VL Regions with Different Fc Formats (hIgG1 LALA (BDG38.145) Vs LALAPG (BDG38.138)

Objective: To examine the functional properties of antibodies having the same VH/VL regions as clone 38.138 but having two different Fc formats. The two different Fc formats comprised mutations in the Fc region that result in reduced binding to the Fc receptor and the inability of the IgG1 to bind to antibody-dependent cellular cytotoxicity components. The formats for comparison were 38.138 (hIgG1 LALA-PG) and 38.145 (hIgG1 LALA). BDG38.138 and BSG38.145 share the same VH and VL sequences, and only differ in the Fc mutations.

Methods

PBMCs activation and treatment—Peripheral blood mononuclear cells (PBMCs) (Cell Generation, USA; CAT: 10102510) were used to determine hIL-13 and hTLSP inhibition by the antibodies. PBMCs were thawed and cultured in growth medium comprising of RPMI—1640, 10% PBS, 1% Glutamax, 1% Sodium-Pyruvate, 0.1% 2-ME, 1% Pen-Strep and 1% nonessential AA. The cells were seeded in 96 well plate at 5×10⁵ cells/well and were allowed to rest for 1 hour at 37° C., 5% CO₂. hIL-13-His (Acro Biosystems, USA; CAT: IL3-H52H4) at a concentration of 0.7 nM, hTSLP-His (Acro Biosystems, USA; CAT: TSP-H52Hb) at a concentration of 70 pM, or a combination of both cytokines at these concentrations were added to the cells, followed immediately by addition of antibodies in serial dilutions, to a total of 200 uL/well. Cells were incubated for 48 hours at 37° C., 5% CO₂.

CD23 expression in monocytes—IC₅₀ of antibody inhibition of hIL-13 was determined by measuring CD23 expression level in monocytes. At the end of 48 hours incubation of the cells with different concentrations of antibodies, monocytes were detached from the bottom of the wells using cold PBS and scraping. Cells were labeled using anti-CD3 (BD Bioscience, USA; CAT: 562280), anti-CD14 (BioLegend, USA; CAT: 301814), anti-CD19 (BioLegend, USA; CAT: 302212) and anti-CD23 (BioLegend, USA; CAT: 338506) antibodies. CD23 percentage of CD14+ population was measured using CytoFLEX flow cytometer (Beckman Coulter, USA).

TARC levels in PBMCs supernatant—TARC levels were determined using TARC DUOSET ELISA kit (R&D systems, USA; CAT: DY364) according to the manufacturer's instructions. Briefly, ELISA high binding protein plates were coated with capture antibody, diluted in PBS. Plates were sealed and incubated overnight at 4° C. The following day plates were washed and blocked using 1% BSA in PBS at room temperature with shaking for 1 hour. The plates were washed three times in PBST, and supernatant from the PBMCs plates was diluted 1:1 in 1% BSA in PBS, transferred to the wells and incubated at room temperature with shaking for 2 hours. The plates were washed three times in PBST. Detection was performed using the kit's detection antibody (biotinylated) in PBS 1% BSA at room temperature with shaking for 2 hours followed by three washes in PBST and addition of Streptavidin-HRP in PBS 1% BSA for 30 minutes. Following 3 washes in PBST, the plates were developed by adding TMB and TMB-stop solution, and 450 nm. Values were analyzed using standard sample curve.

Results

The effect of inhibition of IL-13 and TSLP by either BDG38.138 or BDG38.145 antibodies was tested on human PBMCs. Total PBMC culture includes different cell types such as lymphocytes (including CD4+ T cells, Innate lymphoid cells (ILCs)), and monocytes. The effect of IL-13 and TSLP was assessed by measuring two readouts: CD23 expression levels on monocytes and TARC levels in PBMCs supernatant. The results for BDG38.138 are presented in Example 6 FIGS. 32A and 32B.

CD23 (FcεRII) is the “low affinity” receptor for IgE. Stimulation of PBMCs with IL-13 induces expression of CD23 in B cells and monocytes (May et al., (2012) Preclinical development of CAT-354, an IL-13 neutralizing antibody, for the treatment of severe uncontrolled asthma. British Journal of Pharmacology (2012) 166 177-193).

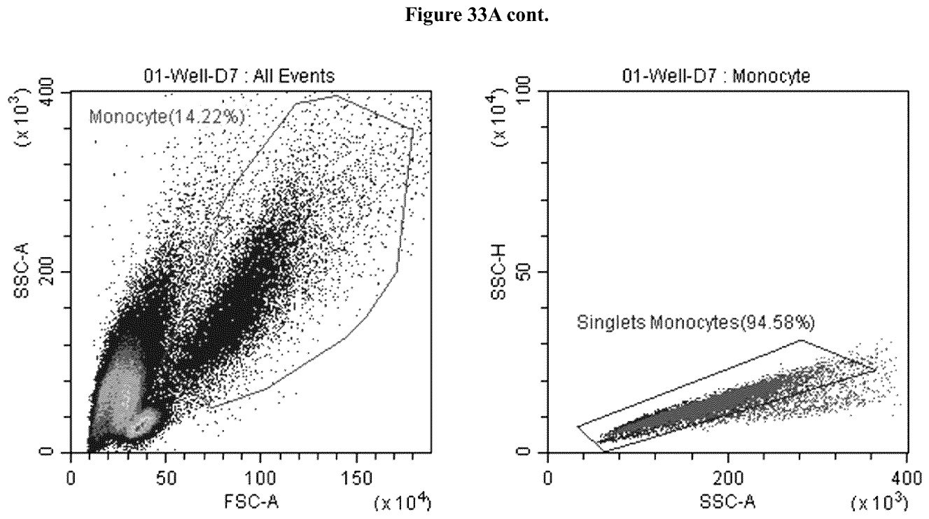

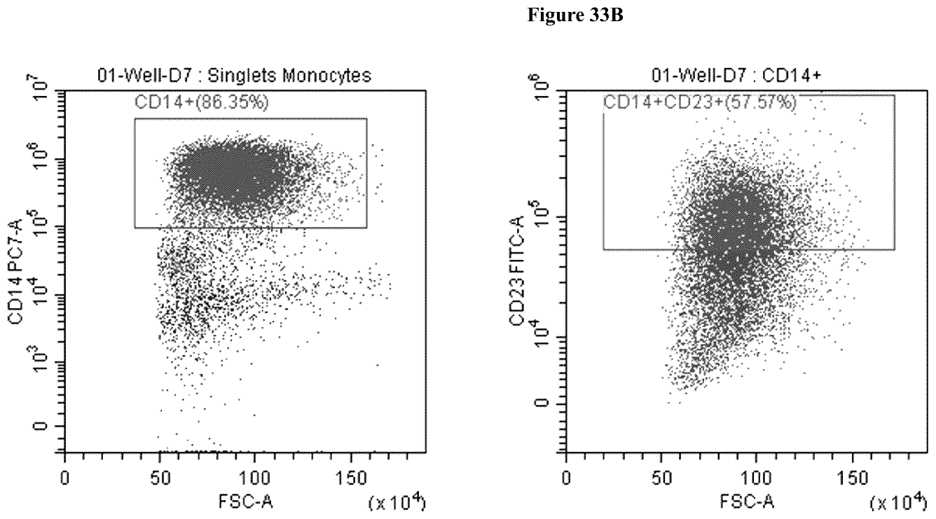

hPBMCs were stimulated with 0.7 nM IL-13 and treated with antibody 38.145 (LALA version), anti-IL-13, or anti-TSLP benchmarks (anti-IL13 and anti-TSLP positive controls) in increasing amounts ranging from 00 nM to 50 nM. After 48 hours of incubation the cells were analyzed by flow cytometry for CD23 expression on monocytes (defined as CD14+ cells) (FIG. 33). BDG 38.145 antibody's ability to neutralize IL-13 was better than anti-IL-13 Ab tralokinumab, the IC50 values being 0.38 nM and 0.91 nM, respectively. The IC50 value of lebrikizumab (anti-TSLP) is 0.38 nM, similar to the IC50 value of the 38.145 antibody.

TARC (Thymus and Activation Regulated Chemokine, CCL17) is a chemokine produced in the thymus and by antigen-presenting cells like dendritic cells, macrophages, and monocytes. TARC-mediated recruitment of Th2 cells and CLA+ CD4+ T cells plays a key role in allergic diseases and serves as a clinical biomarker for AD severity as well as efficacy of treatment. Both IL-13 and TSLP directly upregulate TARC production by dendritic cells.

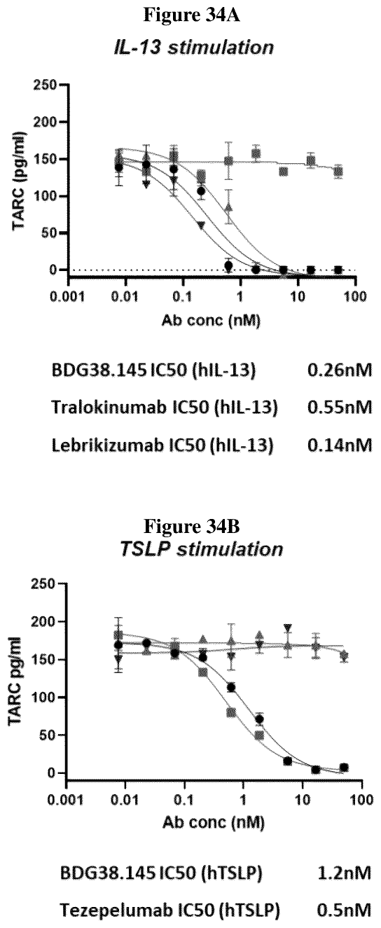

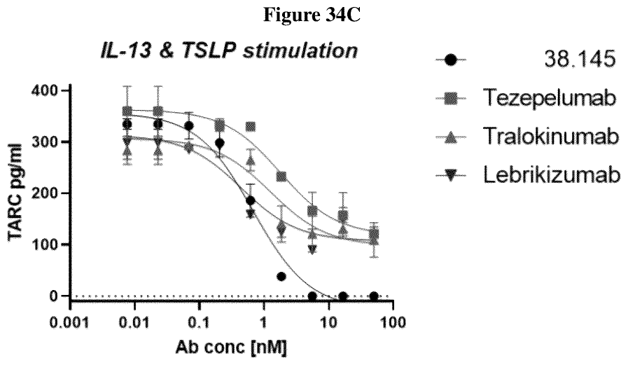

hPBMCs were stimulated with either 0.7 nm IL-13, 70 pM TSLP or with the combination of both cytokines, and treated with BDG38.138 and BDG38.145 antibodies, anti-IL-13, or anti-TSLP positive controls, in increasing amounts ranging from 0 nM to 50 nM. After 48 hours of incubation supernatant was collected to test TARC levels by ELISA (FIGS. 34A-34C).

When cells were stimulated with IL-13 alone (FIG. 34A), treatment with anti-IL-13 antibodies or 38.145 reduced TARC levels to 0, while anti-TSLP antibody, Tezepelumab, had no effect on TARC levels. According to these results, 38.145 inhibits IL-13 signaling in hPBMCs better than tralokinumab and similar to lebrikizumab. When cells were stimulated with TSLP alone (FIG. 34B), treatment with anti-TSLP Ab or 38.145 antibody reduced TARC levels to 0, while anti IL-13 Abs had no effect on TARC levels. This demonstrates that 38.145 inhibits TSLP signaling comparably with Tezepelumab. When cells were stimulated with both cytokines (FIG. 34C), the additive effect of IL-13 and TSLP on TARC levels can be seen in the maximal TARC levels, reaching ˜320 pg/ml. Treatment with either anti IL-13 Abs or anti TSLP antibodies shows only partial inhibition of TARC levels, while treatment with BDG38.145 lowered TARC levels to 0. These results demonstrate the dual blockade of IL-13 and TSLP, where BDG38.145 completely downregulated TARC levels in IL-13 and TSLP stimulated hPBMCs, while antibodies targeting each cytokine demonstrate partial inhibition.

What is claimed is:

1. An isolated anti-IL-13, anti-TSLP dual binding antibody comprising a heavy chain variable region (VH) and a light chain variable region (VL), wherein said VH comprises heavy chain complementarity determining regions (HCDRs) HCDR1, HCDR2, and HCDR3, and said VL comprises light chain complementarity determining regions (LCDRs) LCDR1, LCDR2, and LCDR3, wherein
(a) the HCDR1 comprises the amino acid sequence set forth in any one of SEQ ID NOs: 349, 355, 149 and 136;
(b) the HCDR2 comprises the amino acid sequence set forth in any one of SEQ ID NOs: 350, 352, 354, 356, and 150 or the sequence set forth as: I HX1 Y D G S N K (SEQ ID NO: 142), wherein HX1 is any amino acid;
(c) the HCDR3 comprises the amino acid sequence set forth in any one of SEQ ID NOs: 351, 353, 357, 358, and 151 or the sequence set forth as: A R HX2 HX3 HX4 HX5 HX6 HX7 HX8 HX9 HX10 HX11 F D HX12 (SEQ ID NO: 143), wherein HX2, HX3, HX4, HX5, HX6, HX7, HX8, HX9, HX10, HX11, and HX12 are any amino acid;
(d) the LCDR1 comprises the amino acid sequence of one of SEQ ID NOs: 364, 359, 362, 366, 369, 375, and 152 or the sequence set forth as LX1, LX2, G S K LX3 V (SEQ ID NO: 144), wherein LX1, LX2, and LX3 are any amino acid;
(e) the LCDR2 comprises the amino acid sequence of DDV or DDS, or the sequence set forth as D D LX4, wherein LX4 is any amino acid; and
(f) the LCDR3 comprises the amino acid sequence set forth in any one of SEQ ID NOs: 384, 361, 363, 365, 368, 370-374, 376-383, 385-407, and 154 or the sequence set forth as Q V W D LX5 LX6 S D LX7 V V (SEQ ID NO; 146), wherein LX5, LX6, and LX7 are any amino acid.

2. The isolated dual binding antibody of claim 1, wherein the amino acid sequences of the HCDRs and LCDRs of said antibody are selected from any one of the following sets:
(a) HCDR1 comprises the amino acid sequence set forth in SEQ ID NO: 349, HCDR2 comprises the amino acid sequence set forth in SEQ ID NO: 350, HCDR3 comprises the amino acid sequence set forth in SEQ ID NO: 351, LCDR1 comprises the amino acid sequence set forth in SEQ ID NO: 364, LCDR2 comprises the amino acid sequence DDV, and LCDR3 comprises the amino acid sequence set forth in SEQ ID NO: 384;
(b) HCDR1 comprises the amino acid sequence set forth in SEQ ID NO: 349, HCDR2 comprises the amino acid sequence set forth in SEQ ID NO: 350, HCDR3 comprises the amino acid sequence set forth in SEQ ID NO: 351, LCDR1 comprises the amino acid sequence set forth in SEQ ID NO: 359, LCDR2 comprises the amino acid sequence DDV, and LCDR3 comprises the amino acid sequence set forth in SEQ ID NO: 361;
(c) HCDR1 comprises the amino acid sequence set forth in SEQ ID NO: 349, HCDR2 comprises the amino acid sequence set forth in SEQ ID NO: 356, HCDR3 comprises the amino acid sequence set forth in SEQ ID NO: 351, LCDR1 comprises the amino acid sequence set forth in SEQ ID NO: 364, LCDR2 comprises the amino acid sequence DDV, and LCDR3 comprises the amino acid sequence set forth in SEQ ID NO: 371;
(d) HCDR1 comprises the amino acid sequence set forth in SEQ ID NO: 349, HCDR2 comprises the amino acid sequence set forth in SEQ ID NO: 350, HCDR3 comprises the amino acid sequence set forth in SEQ ID NO: 351, LCDR1 comprises the amino acid sequence set forth in SEQ ID NO: 362, LCDR2 comprises the amino acid sequence DDV, and LCDR3 comprises the amino acid sequence set forth in SEQ ID NO: 384;
(e) HCDR1 comprises the amino acid sequence set forth in SEQ ID NO: 349, HCDR2 comprises the amino acid sequence set forth in SEQ ID NO: 352, HCDR3 comprises the amino acid sequence set forth in SEQ ID NO: 353, LCDR1 comprises the amino acid sequence set forth in SEQ ID NO: 362, LCDR2 comprises the amino acid sequence DDV, and LCDR3 comprises the amino acid sequence set forth in SEQ ID NO: 363;
(f) HCDR1 comprises the amino acid sequence set forth in SEQ ID NO: 349, HCDR2 comprises the amino acid sequence set forth in SEQ ID NO: 354, HCDR3 comprises the amino acid sequence set forth in SEQ ID NO: 351, LCDR1 comprises the amino acid sequence set forth in SEQ ID NO: 364, LCDR2 comprises the amino acid sequence DDV, and LCDR3 comprises the amino acid sequence set forth in SEQ ID NO: 365;
(g) HCDR1 comprises the amino acid sequence set forth in SEQ ID NO: 349, HCDR2 comprises the amino acid sequence set forth in SEQ ID NO: 352, HCDR3 comprises the amino acid sequence set forth in SEQ ID NO: 351, LCDR1 comprises the amino acid sequence set forth in SEQ ID NO: 366, LCDR2 comprises the amino acid sequence DDS, and LCDR3 comprises the amino acid sequence set forth in SEQ ID NO: 368;
(h) HCDR1 comprises the amino acid sequence set forth in SEQ ID NO: 355, HCDR2 comprises the amino acid sequence set forth in SEQ ID NO: 356, HCDR3 comprises the amino acid sequence set forth in SEQ ID NO: 351, LCDR1 comprises the amino acid sequence set forth in SEQ ID NO: 369, LCDR2 comprises the amino acid sequence DDS, and LCDR3 comprises the amino acid sequence set forth in SEQ ID NO: 370;
(i) HCDR1 comprises the amino acid sequence set forth in SEQ ID NO: 349, HCDR2 comprises the amino acid sequence set forth in SEQ ID NO: 352, HCDR3 comprises the amino acid sequence set forth in SEQ ID NO: 353, LCDR1 comprises the amino acid sequence set forth in SEQ ID NO: 362, LCDR2 comprises the amino acid sequence DDS, and LCDR3 comprises the amino acid sequence set forth in SEQ ID NO: 372;
(j) HCDR1 comprises the amino acid sequence set forth in SEQ ID NO: 349, HCDR2 comprises the amino acid sequence set forth in SEQ ID NO: 350, HCDR3 comprises the amino acid sequence set forth in SEQ ID NO: 351, LCDR1 comprises the amino acid sequence set forth in SEQ ID NO: 359, LCDR2 comprises the amino acid sequence DDV, and LCDR3 comprises the amino acid sequence set forth in SEQ ID NO: 373;
(k) HCDR1 comprises the amino acid sequence set forth in SEQ ID NO: 349, HCDR2 comprises the amino acid sequence set forth in SEQ ID NO: 350, HCDR3 comprises the amino acid sequence set forth in SEQ ID NO: 351, LCDR1 comprises the amino acid sequence set forth in SEQ ID NO: 364, LCDR2 comprises the amino acid sequence DDV, and LCDR3 comprises the amino acid sequence set forth in SEQ ID NO: 374;
(l) HCDR1 comprises the amino acid sequence set forth in SEQ ID NO: 355, HCDR2 comprises the amino acid sequence set forth in SEQ ID NO: 350, HCDR3 comprises the amino acid sequence set forth in SEQ ID NO: 357, LCDR1 comprises the amino acid sequence set forth in SEQ ID NO: 375, LCDR2 comprises the amino acid sequence DDS, and LCDR3 comprises the amino acid sequence set forth in SEQ ID NO: 376;
(m) HCDR1 comprises the amino acid sequence set forth in SEQ ID NO: 349, HCDR2 comprises the amino acid sequence set forth in SEQ ID NO: 350, HCDR3 comprises the amino acid sequence set forth in SEQ ID NO: 351, LCDR1 comprises the amino acid sequence set forth in SEQ ID NO: 362, LCDR2 comprises the amino acid sequence DDV, and LCDR3 comprises the amino acid sequence set forth in SEQ ID NO: 377;
(n) HCDR1 comprises the amino acid sequence set forth in SEQ ID NO: 355, HCDR2 comprises the amino acid sequence set forth in SEQ ID NO: 356, HCDR3 comprises the amino acid sequence set forth in SEQ ID NO: 351, LCDR1 comprises the amino acid sequence set forth in SEQ ID NO: 369, LCDR2 comprises the amino acid sequence DDS, and LCDR3 comprises the amino acid sequence set forth in SEQ ID NO: 378;
(o) HCDR1 comprises the amino acid sequences amino acid sequence set forth in SEQ ID NO: 355, HCDR2 comprises the amino acid sequence set forth in SEQ ID NO: 356, HCDR3 comprises the amino acid sequence set forth in SEQ ID NO: 351, LCDR1 comprises the amino acid sequence set forth in SEQ ID NO: 369, LCDR2 comprises the amino acid sequence DDS, and LCDR3 comprises the amino acid sequence set forth in SEQ ID NO: 379;
(p) HCDR1 comprises the amino acid sequence set forth in SEQ ID NO: 349, HCDR2 comprises the amino acid sequence set forth in SEQ ID NO: 350, HCDR3 comprises the amino acid sequence set forth in SEQ ID NO: 351, LCDR1 comprises the amino acid sequence set forth in SEQ ID NO: 359, LCDR2 comprises the amino acid sequence DDV, and LCDR3 comprises the amino acid sequence set forth in SEQ ID NO: 380;
(q) HCDR1 comprises the amino acid sequence set forth in SEQ ID NO: 355, HCDR2 comprises the amino acid sequence set forth in SEQ ID NO: 356, HCDR3 comprises the amino acid sequence set forth in SEQ ID NO: 351, LCDR1 comprises the amino acid sequence set forth in SEQ ID NO: 369, LCDR2 comprises the amino acid sequence DDS, and LCDR3 comprises the amino acid sequence set forth in SEQ ID NO: 379;
(r) HCDR1 comprises the amino acid sequence set forth in SEQ ID NO: 355, HCDR2 comprises the amino acid sequence set forth in SEQ ID NO: 352, HCDR3 comprises the amino acid sequence set forth in SEQ ID NO: 351, LCDR1 comprises the amino acid sequence set forth in SEQ ID NO: 369, LCDR2 comprises the amino acid sequence DDS, and LCDR3 comprises the amino acid sequence set forth in SEQ ID NO: 381;
(s) HCDR1 comprises the amino acid sequence set forth in SEQ ID NO: 355, HCDR2 comprises the amino acid sequence set forth in SEQ ID NO: 350, HCDR3 comprises the amino acid sequence set forth in SEQ ID NO: 357, LCDR1 comprises the amino acid sequence set forth in SEQ ID NO: 369, LCDR2 comprises the amino acid sequence DDS, and LCDR3 comprises the amino acid sequence set forth in SEQ ID NO: 370;
(t) HCDR1 comprises the amino acid sequence set forth in SEQ ID NO: 355, HCDR2 comprises the amino acid sequence set forth in SEQ ID NO: 356, HCDR3 comprises the amino acid sequence set forth in SEQ ID NO: 351, LCDR1 comprises the amino acid sequence set forth in SEQ ID NO: 366, LCDR2 comprises the amino acid sequence DDS, and LCDR3 comprises the amino acid sequence set forth in SEQ ID NO: 382;
(u) HCDR1 comprises the amino acid sequence set forth in SEQ ID NO: 355, HCDR2 comprises the amino acid sequence set forth in SEQ ID NO: 350, HCDR3 comprises the amino acid sequence set forth in SEQ ID NO: 351, LCDR1 comprises the amino acid sequence set forth in SEQ ID NO: 369, LCDR2 comprises the amino acid sequence DDS, and LCDR3 comprises the amino acid sequence set forth in SEQ ID NO: 370;
(v) HCDR1 comprises the amino acid sequence set forth in SEQ ID NO: 355, HCDR2 comprises the amino acid sequence set forth in SEQ ID NO: 356, HCDR3 comprises the amino acid sequence set forth in SEQ ID NO: 351, LCDR1 comprises the amino acid sequence set forth in SEQ ID NO: 369, LCDR2 comprises the amino acid sequence DDS, and LCDR3 comprises the amino acid sequence set forth in SEQ ID NO: 383;
(w) HCDR1 comprises the amino acid sequence set forth in SEQ ID NO: 355, HCDR2 comprises the amino acid sequence set forth in SEQ ID NO: 352, HCDR3 comprises the amino acid sequence set forth in SEQ ID NO: 351, LCDR1 comprises the amino acid sequence set forth in SEQ ID NO: 366, LCDR2 comprises the amino acid sequence DDS, and LCDR3 comprises the amino acid sequence set forth in SEQ ID NO: 385;
(x) HCDR1 comprises the amino acid sequence set forth in SEQ ID NO: 355, HCDR2 comprises the amino acid sequence set forth in SEQ ID NO: 350, HCDR3 comprises the amino acid sequence set forth in SEQ ID NO: 351, LCDR1 comprises the amino acid sequence set forth in SEQ ID NO: 369, LCDR2 comprises the amino acid sequence DDS, and LCDR3 comprises the amino acid sequence set forth in SEQ ID NO: 386;
(y) HCDR1 comprises the amino acid sequence set forth in SEQ ID NO: 355, HCDR2 comprises the amino acid sequence set forth in SEQ ID NO: 352, HCDR3 comprises the amino acid sequence set forth in SEQ ID NO: 351, LCDR1 comprises the amino acid sequence set forth in SEQ ID NO: 369, LCDR2 comprises the amino acid sequence DDS, and LCDR3 comprises the amino acid sequence set forth in SEQ ID NO: 387;
(z) HCDR1 comprises the amino acid sequence set forth in SEQ ID NO: 355, HCDR2 comprises the amino acid sequence set forth in SEQ ID NO: 356, HCDR3 comprises the amino acid sequence set forth in SEQ ID NO: 351, LCDR1 comprises the amino acid sequence set forth in SEQ ID NO: 369, LCDR2 comprises the amino acid sequence DDS, and LCDR3 comprises the amino acid sequence set forth in SEQ ID NO: 388;
(aa) HCDR1 comprises the amino acid sequence set forth in SEQ ID NO: 355, HCDR2 comprises the amino acid sequence set forth in SEQ ID NO: 356, HCDR3 comprises the amino acid sequence set forth in SEQ ID NO: 351, LCDR1 comprises the amino acid sequence set forth in SEQ ID NO: 369, LCDR2 comprises the amino acid sequence DDS, and LCDR3 comprises the amino acid sequence set forth in SEQ ID NO: 389;
(bb) HCDR1 comprises the amino acid sequence set forth in SEQ ID NO: 355, HCDR2 comprises the amino acid sequence set forth in SEQ ID NO: 352, HCDR3 comprises the amino acid sequence set forth in SEQ ID NO: 351, LCDR1 comprises the amino acid sequence set forth in SEQ ID NO: 369, LCDR2 comprises the amino acid sequence DDS, and LCDR3 comprises the amino acid sequence set forth in SEQ ID NO: 379;
(cc) HCDR1 comprises the amino acid sequence set forth in SEQ ID NO: 355, HCDR2 comprises the amino acid sequence set forth in SEQ ID NO: 352, HCDR3 comprises the amino acid sequence set forth in SEQ ID NO: 351, LCDR1 comprises the amino acid sequence set forth in SEQ ID NO: 369, LCDR2 comprises the amino acid sequence DDS, and LCDR3 comprises the amino acid sequence set forth in SEQ ID NO: 390;
(dd) HCDR1 comprises the amino acid sequence set forth in SEQ ID NO: 355, HCDR2 comprises the amino acid sequence set forth in SEQ ID NO: 350, HCDR3 comprises the amino acid sequence set forth in SEQ ID NO: 351, LCDR1 comprises the amino acid sequence set forth in SEQ ID NO: 366, LCDR2 comprises the amino acid sequence DDS, and LCDR3 comprises the amino acid sequence set forth in SEQ ID NO: 391;
(ee) HCDR1 comprises the amino acid sequence set forth in SEQ ID NO: 355, HCDR2 comprises the amino acid sequence set forth in SEQ ID NO: 350, HCDR3 comprises the amino acid sequence set forth in SEQ ID NO: 351, LCDR1 comprises the amino acid sequence set forth in SEQ ID NO: 375, LCDR2 comprises the amino acid sequence DDS, and LCDR3 comprises the amino acid sequence set forth in SEQ ID NO: 392;
(ff) HCDR1 comprises the amino acid sequence set forth in SEQ ID NO: 355, HCDR2 comprises the amino acid sequence set forth in SEQ ID NO: 354, HCDR3 comprises the amino acid sequence set forth in SEQ ID NO: 351, LCDR1 comprises the amino acid sequence set forth in SEQ ID NO: 369, LCDR2 comprises the amino acid sequence DDS, and LCDR3 comprises the amino acid sequence set forth in SEQ ID NO: 393;
(gg) HCDR1 comprises the amino acid sequence set forth in SEQ ID NO: 355, HCDR2 comprises the amino acid sequence set forth in SEQ ID NO: 356, HCDR3 comprises the amino acid sequence set forth in SEQ ID NO: 351, LCDR1 comprises the amino acid sequence set forth in SEQ ID NO: 369, LCDR2 comprises the amino acid sequence DDS, and LCDR3 comprises the amino acid sequence set forth in SEQ ID NO: 394;
(hh) HCDR1 comprises the amino acid sequence set forth in SEQ ID NO: 355, HCDR2 comprises the amino acid sequence set forth in SEQ ID NO: 356, HCDR3 comprises the amino acid sequence set forth in SEQ ID NO: 351, LCDR1 comprises the amino acid sequence set forth in SEQ ID NO: 369, LCDR2 comprises the amino acid sequence DDS, and LCDR3 comprises the amino acid sequence set forth in SEQ ID NO: 395;
(ii) HCDR1 comprises the amino acid sequence set forth in SEQ ID NO: 355, HCDR2 comprises the amino acid sequence set forth in SEQ ID NO: 350, HCDR3 comprises the amino acid sequence set forth in SEQ ID NO: 351, LCDR1 comprises the amino acid sequence set forth in SEQ ID NO: 369, LCDR2 comprises the amino acid sequence DDS, and LCDR3 comprises the amino acid sequence set forth in SEQ ID NO: 396;
(jj) HCDR1 comprises the amino acid sequence set forth in SEQ ID NO: 355, HCDR2 comprises the amino acid sequence set forth in SEQ ID NO: 352, HCDR3 comprises the amino acid sequence set forth in SEQ ID NO: 351, LCDR1 comprises the amino acid sequence set forth in SEQ ID NO: 369, LCDR2 comprises the amino acid sequence DDS, and LCDR3 comprises the amino acid sequence set forth in SEQ ID NO: 397;
(kk) HCDR1 comprises the amino acid sequence set forth in SEQ ID NO: 355, HCDR2 comprises the amino acid sequence set forth in SEQ ID NO: 350, HCDR3 comprises the amino acid sequence set forth in SEQ ID NO: 351, LCDR1 comprises the amino acid sequence set forth in SEQ ID NO: 369, LCDR2 comprises the amino acid sequence DDS, and LCDR3 comprises the amino acid sequence set forth in SEQ ID NO: 398;
(ll) HCDR1 comprises the amino acid sequence set forth in SEQ ID NO: 355, HCDR2 comprises the amino acid sequence set forth in SEQ ID NO: 350, HCDR3 comprises the amino acid sequence set forth in SEQ ID NO: 351, LCDR1 comprises the amino acid sequence set forth in SEQ ID NO: 369, LCDR2 comprises the amino acid sequence DDS, and LCDR3 comprises the amino acid sequence set forth in SEQ ID NO: 388;
(mm) HCDR1 comprises the amino acid sequence set forth in SEQ ID NO: 355, HCDR2 comprises the amino acid sequence set forth in SEQ ID NO: 350, HCDR3 comprises the amino acid sequence set forth in SEQ ID NO: 351, LCDR1 comprises the amino acid sequence set forth in SEQ ID NO: 369, LCDR2 comprises the amino acid sequence DDS, and LCDR3 comprises the amino acid sequence set forth in SEQ ID NO: 394;
(nn) HCDR1 comprises the amino acid sequence set forth in SEQ ID NO: 355, HCDR2 comprises the amino acid sequence set forth in SEQ ID NO: 350, HCDR3 comprises the amino acid sequence set forth in SEQ ID NO: 351, LCDR1 comprises the amino acid sequence set forth in SEQ ID NO: 375, LCDR2 comprises the amino acid sequence DDS, and LCDR3 comprises the amino acid sequence set forth in SEQ ID NO: 398;
(oo) HCDR1 comprises the amino acid sequence set forth in SEQ ID NO: 355, HCDR2 comprises the amino acid sequence set forth in SEQ ID NO: 352, HCDR3 comprises the amino acid sequence set forth in SEQ ID NO: 357, LCDR1 comprises the amino acid sequence set forth in SEQ ID NO: 375, LCDR2 comprises the amino acid sequence DDS, and LCDR3 comprises the amino acid sequence set forth in SEQ ID NO: 399;
(pp) HCDR1 is set forth in SEQ ID NO: 355, HCDR2 comprises the amino acid sequence set forth in SEQ ID NO: 350, HCDR3 comprises the amino acid sequence set forth in SEQ ID NO: 351, LCDR1 comprises the amino acid sequence set forth in SEQ ID NO: 375, LCDR2 comprises the amino acid sequence DDS, and LCDR3 comprises the amino acid sequence set forth in SEQ ID NO: 400;
(qq) HCDR1 comprises the amino acid sequence set forth in SEQ ID NO: 355, HCDR2 comprises the amino acid sequence set forth in SEQ ID NO: 350, HCDR3 comprises the amino acid sequence set forth in SEQ ID NO: 357, LCDR1 comprises the amino acid sequence set forth in SEQ ID NO: 375, LCDR2 comprises the amino acid sequence DDS, and LCDR3 comprises the amino acid sequence set forth in SEQ ID NO: 398;
(rr) HCDR1 comprises the amino acid sequence set forth in SEQ ID NO: 349, HCDR2 comprises the amino acid sequence set forth in SEQ ID NO: 350, HCDR3 comprises the amino acid sequence set forth in SEQ ID NO: 351, LCDR1 comprises the amino acid sequence set forth in SEQ ID NO: 369, LCDR2 comprises the amino acid sequence DDS, and LCDR3 comprises the amino acid sequence set forth in SEQ ID NO: 378;
(ss) HCDR1 comprises the amino acid sequence set forth in SEQ ID NO: 355, HCDR2 comprises the amino acid sequence set forth in SEQ ID NO: 350, HCDR3 comprises the amino acid sequence set forth in SEQ ID NO: 351, LCDR1 comprises the amino acid sequence set forth in SEQ ID NO: 375, LCDR2 comprises the amino acid sequence DDS, and LCDR3 comprises the amino acid sequence set forth in SEQ ID NO: 401;
(tt) HCDR1 comprises the amino acid sequence set forth in SEQ ID NO: 355, HCDR2 comprises the amino acid sequence set forth in SEQ ID NO: 356, HCDR3 comprises the amino acid sequence set forth in SEQ ID NO: 358, LCDR1 comprises the amino acid sequence set forth in SEQ ID NO: 369, LCDR2 comprises the amino acid sequence DDS, and LCDR3 comprises the amino acid sequence set forth in SEQ ID NO: 402;
(uu) HCDR1 comprises the amino acid sequence set forth in SEQ ID NO: 349, HCDR2 comprises the amino acid sequence set forth in SEQ ID NO: 356, HCDR3 comprises the amino acid sequence set forth in SEQ ID NO: 358, LCDR1 comprises the amino acid sequence set forth in SEQ ID NO: 369, LCDR2 comprises the amino acid sequence DDS, and LCDR3 comprises the amino acid sequence set forth in SEQ ID NO: 403;
(vv) HCDR1 comprises the amino acid sequence set forth in SEQ ID NO: 355, HCDR2 comprises the amino acid sequence set forth in SEQ ID NO: 356, HCDR3 comprises the amino acid sequence set forth in SEQ ID NO: 358, LCDR1 comprises the amino acid sequence set forth in SEQ ID NO: 375, LCDR2 comprises the amino acid sequence DDS, and LCDR3 comprises the amino acid sequence set forth in SEQ ID NO: 376;
(ww) HCDR1 comprises the amino acid sequence set forth in SEQ ID NO: 355, HCDR2 comprises the amino acid sequence set forth in SEQ ID NO: 350, HCDR3 comprises the amino acid sequence set forth in SEQ ID NO: 351, LCDR1 comprises the amino acid sequence set forth in SEQ ID NO: 369, LCDR2 comprises the amino acid sequence DDS, and LCDR3 comprises the amino acid sequence set forth in SEQ ID NO: 402;
(xx) HCDR1 comprises the amino acid sequence set forth in SEQ ID NO: 349, HCDR2 comprises the amino acid sequence set forth in SEQ ID NO: 350, HCDR3 comprises the amino acid sequence set forth in SEQ ID NO: 353, LCDR1 comprises the amino acid sequence set forth in SEQ ID NO: 359, LCDR2 comprises the amino acid sequence DDV, and LCDR3 comprises the amino acid sequence set forth in SEQ ID NO: 404;
(yy) HCDR1 comprises the amino acid sequence set forth in SEQ ID NO: 355, HCDR2 comprises the amino acid sequence set forth in SEQ ID NO: 350, HCDR3 comprises the amino acid sequence set forth in SEQ ID NO: 357, LCDR1 comprises the amino acid sequence set forth in SEQ ID NO: 375, LCDR2 comprises the amino acid sequence DDS, and LCDR3 comprises the amino acid sequence set forth in SEQ ID NO: 405;
(zz) HCDR1 comprises the amino acid sequence set forth in SEQ ID NO: 355, HCDR2 comprises the amino acid sequence set forth in SEQ ID NO: 350, HCDR3 comprises the amino acid sequence set forth in SEQ ID NO: 351, LCDR1 comprises the amino acid sequence set forth in SEQ ID NO: 366, LCDR2 comprises the amino acid sequence DDS, and LCDR3 comprises the amino acid sequence set forth in SEQ ID NO: 406;
(aaa) HCDR1 comprises the amino acid sequence set forth in SEQ ID NO: 355, HCDR2 comprises the amino acid sequence set forth in SEQ ID NO: 350, HCDR3 comprises the amino acid sequence set forth in SEQ ID NO: 351, LCDR1 comprises the amino acid sequence set forth in SEQ ID NO: 375, LCDR2 comprises the amino acid sequence DDS, and LCDR3 comprises the amino acid sequence set forth in SEQ ID NO: 407; and
(bbb) HCDR1 comprises the amino acid sequence set forth in SEQ ID NO: 355, HCDR2 comprises the amino acid sequence set forth in SEQ ID NO: 354, HCDR3 comprises the amino acid sequence set forth in SEQ ID NO: 351, LCDR1 comprises the amino acid sequence set forth in SEQ ID NO: 369, LCDR2 comprises the amino acid sequence DDS, and LCDR3 comprises the amino acid sequence set forth in SEQ ID NO: 379.

3. The isolated dual binding antibody of claim 1, wherein said VH and said VL comprise the amino acid sequences set forth in SEQ ID Nos:337 and 338, respectively; SEQ ID NOs:209 and 210, respectively; SEQ ID NOs:219 and 220, respectively; SEQ ID NOs:249 and 250, respectively; respectively; SEQ ID NOs:155 and 156, respectively; SEQ ID NOs:157 and 158, respectively; SEQ ID NOs:4 and 3, respectively; SEQ ID NOs:6 and 5, respectively; SEQ ID NOs:8 and 7, respectively; SEQ ID NOs:10 and 9, respectively; SEQ ID NOs:12 and 11, respectively; SEQ ID NOs:14 and 13, respectively; SEQ ID NOs:16 and 15, respectively; SEQ ID NOs:18 and 17, respectively; SEQ ID NOs:20 and 19, respectively; SEQ ID NOs:22 and 21, respectively; SEQ ID NOs:24 and 23, respectively; SEQ ID NOs:26 and 25, respectively; SEQ ID NOs:28 and 27, respectively; SEQ ID NOs:30 and 29, respectively; SEQ ID NOs:32 and 31, respectively; SEQ ID NOs:34 and 33, respectively; SEQ ID NOs:36 and 35, respectively; SEQ ID NOs:38 and 37, respectively; SEQ ID NOs:40 and 39, respectively; SEQ ID NOs:42 and 41, respectively; SEQ ID NOs:44 and 43, respectively; SEQ ID NOs:46 and 45, respectively; SEQ ID NOs:48 and 47, respectively; SEQ ID NOs:50 and 49, respectively; SEQ ID NOs:52 and 51, respectively; or SEQ ID NOs:54 and 53, respectively.

4. The isolated dual binding antibody of claim 1, wherein said antibody comprises an IgG, an Fv, an scFv, an Fab, an F(ab′)₂, a minibody, a diabody, or a triabody, wherein said IgG is IgG1, IgG2, IgG3, or IgG4.

5. The isolated dual binding antibody of claim 4, wherein said IgG comprises a heavy chain (HC) comprising a mutation that reduces binding to an Fc receptor, wherein said dual binding antibody comprising this mutation is unable to bind to antibody-dependent cellular cytotoxicity components.

6. The isolated dual binding antibody of claim 5, wherein said mutation is a L234A L235A (LALA) mutation or a L234A L235AP329G (LALAPG) mutation.

7. The isolated dual binding antibody of claim 7, wherein said VH and said VL comprise the amino acid sequences set forth in SEQ ID Nos:337 and 338, respectively, and said antibody comprises said L234A L235A (LALA) mutation or said L234A L235AP329G (LALAPG) mutation.

8. The isolated dual binding antibody of claim 7, wherein said antibody comprises a heavy chain (HC) and a light chain (LC), and wherein amino acid sequence of said HC and LC are set forth in SEQ ID NOs: 408 and 409, respectively, or SEQ ID NOs: 410 and 409, respectively.

9. A composition comprising the isolated dual binding antibody of claim 1 and a pharmaceutically acceptable carrier.

10. An isolated polynucleotide construct encoding the isolated dual binding antibody of claim 1.

11. An expression vector comprising the polynucleotide construct of claim 10.

12. A host cell comprising the expression vector of claim 11.

13. An isolated anti-IL-13, anti-TSLP dual binding antibody comprising a heavy chain variable region (VH) and a light chain variable region (VL), wherein said VH comprises heavy chain coplanarity determining regions (HCDRs) HCDR1, HCDR2, and HCDR3, said VL comprises light chain complementarity determining regions (LCDRs) LCDR1, LCDR2, and LCDR3, wherein the HCDR1, HCDR2, and HCDR3 comprise the amino acid sequences of SEQ ID NOs:349, 350, and 351, respectively, and the LCDR1, LCDR2, and LCDR3 comprise the amino acid sequences of SEQ ID NOs:364, 360, and 384, respectively.

14. An isolated anti-IL-13, anti-TSLP dual binding antibody comprising a heavy chain variable region (VH) and a light chain variable region (VL), wherein said VH and said VL comprise the amino acid sequences set forth in SEQ ID Nos:337 and 338, respectively.

15. An isolated anti-IL-13, anti-TSLP dual binding antibody, comprising a heavy chain (HC) comprising a heavy chain variable region (VH) and a light chain (LC) comprising a light chain variable region (VL), wherein said HC comprises a L234A L235A (LALA) mutation or a L234A L235A P329G (LALAPG) mutation, and wherein the amino acid sequences of said VH and VL are set forth in SEQ ID NOs: 337 and 338, respectively.

16. The isolated dual binding antibody of claim 15, wherein amino acid sequences of said HC and LC are set forth in SEQ ID NOs: 410 and 409, respectively, or SEQ ID NOs: 408 and 409, respectively.

17. A method of treating a subject suffering from a disease or condition, said method comprises administering to said subject a composition comprising an isolated anti-IL-13, anti-TSLP dual binding antibody comprising a heavy chain variable region (VH) and a light chain variable region (VL), wherein said VH comprises heavy chain complementarity determining regions (HCDRs) HCDR1, HCDR2, and HCDR3, said VL comprises light chain complementarity determining regions (LCDRs) LCDR1, LCDR2, and LCDR3, wherein
(a) the HCDR1 comprises the amino acid sequence set forth in any one of SEQ ID NOs: 349, 355, 149 and 136;
(b) the HCDR2 comprises the amino acid sequence set forth in any one of SEQ ID NOs: 350, 352, 354, 356, and 150 or the sequence set forth as: I HX1 Y D G S N K (SEQ ID NO: 142), wherein HX1 is any amino acid;
(c) the HCDR3 comprises the amino acid sequence set forth in any one of SEQ ID NOs: 351, 353, 357, 358, and 151 or the sequence set forth as: A R HX2 HX3 HX4 HX5 HX6 HX7 HX8 HX9 HX10 HX11 F D HX12 (SEQ ID NO: 143), wherein HX2, HX3, HX4, HX5, HX6, HX7, HX8, HX9, HX10, HX11, and HX12 are any amino acid;
(d) the LCDR1 comprises the amino acid sequence of one of SEQ ID NOs: 364, 359, 362, 366, 369, 375, and 152 or the sequence set forth as LX1, LX2, G S K LX3 V (SEQ ID NO: 144), wherein LX1, LX2, and LX3 are any amino acid;
(e) the LCDR2 comprises the amino acid sequence of DDV or DDS, or the sequence set forth as D D LX4, wherein LX4 is any amino acid; and
(f) the LCDR3 comprises the amino acid sequence set forth in any one of SEQ ID NOs: 384, 361, 363, 365, 368, 370-374, 376-383, 385-407, and 154 or the sequence set forth as Q V W D LX5 LX6 S D LX7 V V (SEQ ID NO; 146), wherein LX5, LX6, and LX7 are any amino acid.

18. The method of claim 17, wherein the amino acid sequences of the HCDRs and LCDRs of said antibody are selected from any one of the following sets:
(a) HCDR1 comprises the amino acid sequence set forth in SEQ ID NO: 349, HCDR2 comprises the amino acid sequence set forth in SEQ ID NO: 350, HCDR3 comprises the amino acid sequence set forth in SEQ ID NO: 351, LCDR1 comprises the amino acid sequence set forth in SEQ ID NO: 364, LCDR2 comprises the amino acid sequence DDV, and LCDR3 comprises the amino acid sequence set forth in SEQ ID NO: 384;
(b) HCDR1 comprises the amino acid sequence set forth in SEQ ID NO: 349, HCDR2 comprises the amino acid sequence set forth in SEQ ID NO: 350, HCDR3 comprises the amino acid sequence set forth in SEQ ID NO: 351, LCDR1 comprises the amino acid sequence set forth in SEQ ID NO: 359, LCDR2 comprises the amino acid sequence DDV, and LCDR3 comprises the amino acid sequence set forth in SEQ ID NO: 361;
(c) HCDR1 comprises the amino acid sequence set forth in SEQ ID NO: 349, HCDR2 comprises the amino acid sequence set forth in SEQ ID NO: 356, HCDR3 comprises the amino acid sequence set forth in SEQ ID NO: 351, LCDR1 comprises the amino acid sequence set forth in SEQ ID NO: 364, LCDR2 comprises the amino acid sequence DDV, and LCDR3 comprises the amino acid sequence set forth in SEQ ID NO: 371;
(d) HCDR1 comprises the amino acid sequence set forth in SEQ ID NO: 349, HCDR2 comprises the amino acid sequence set forth in SEQ ID NO: 350, HCDR3 comprises the amino acid sequence set forth in SEQ ID NO: 351, LCDR1 comprises the amino acid sequence set forth in SEQ ID NO: 362, LCDR2 comprises the amino acid sequence DDV, and LCDR3 comprises the amino acid sequence set forth in SEQ ID NO: 384;
(e) HCDR1 comprises the amino acid sequence set forth in SEQ ID NO: 349, HCDR2 comprises the amino acid sequence set forth in SEQ ID NO: 352, HCDR3 comprises the amino acid sequence set forth in SEQ ID NO: 353, LCDR1 comprises the amino acid sequence set forth in SEQ ID NO: 362, LCDR2 comprises the amino acid sequence DDV, and LCDR3 comprises the amino acid sequence set forth in SEQ ID NO: 363;
(f) HCDR1 comprises the amino acid sequence set forth in SEQ ID NO: 349, HCDR2 comprises the amino acid sequence set forth in SEQ ID NO: 354, HCDR3 comprises the amino acid sequence set forth in SEQ ID NO: 351, LCDR1 comprises the amino acid sequence set forth in SEQ ID NO: 364, LCDR2 comprises the amino acid sequence DDV, and LCDR3 comprises the amino acid sequence set forth in SEQ ID NO: 365;
(g) HCDR1 comprises the amino acid sequence set forth in SEQ ID NO: 349, HCDR2 comprises the amino acid sequence set forth in SEQ ID NO: 352, HCDR3 comprises the amino acid sequence set forth in SEQ ID NO: 351, LCDR1 comprises the amino acid sequence set forth in SEQ ID NO: 366, LCDR2 comprises the amino acid sequence DDS, and LCDR3 comprises the amino acid sequence set forth in SEQ ID NO: 368;
(h) HCDR1 comprises the amino acid sequence set forth in SEQ ID NO: 355, HCDR2 comprises the amino acid sequence set forth in SEQ ID NO: 356, HCDR3 comprises the amino acid sequence set forth in SEQ ID NO: 351, LCDR1 comprises the amino acid sequence set forth in SEQ ID NO: 369, LCDR2 comprises the amino acid sequence DDS, and LCDR3 comprises the amino acid sequence set forth in SEQ ID NO: 370;
(i) HCDR1 comprises the amino acid sequence set forth in SEQ ID NO: 349, HCDR2 comprises the amino acid sequence set forth in SEQ ID NO: 352, HCDR3 comprises the amino acid sequence set forth in SEQ ID NO: 353, LCDR1 comprises the amino acid sequence set forth in SEQ ID NO: 362, LCDR2 comprises the amino acid sequence DDS, and LCDR3 comprises the amino acid sequence set forth in SEQ ID NO: 372;
(j) HCDR1 comprises the amino acid sequence set forth in SEQ ID NO: 349, HCDR2 comprises the amino acid sequence set forth in SEQ ID NO: 350, HCDR3 comprises the amino acid sequence set forth in SEQ ID NO: 351, LCDR1 comprises the amino acid sequence set forth in SEQ ID NO: 359, LCDR2 comprises the amino acid sequence DDV, and LCDR3 comprises the amino acid sequence set forth in SEQ ID NO: 373;
(k) HCDR1 comprises the amino acid sequence set forth in SEQ ID NO: 349, HCDR2 comprises the amino acid sequence set forth in SEQ ID NO: 350, HCDR3 comprises the amino acid sequence set forth in SEQ ID NO: 351, LCDR1 comprises the amino acid sequence set forth in SEQ ID NO: 364, LCDR2 comprises the amino acid sequence DDV, and LCDR3 comprises the amino acid sequence set forth in SEQ ID NO: 374;
(l) HCDR1 comprises the amino acid sequence set forth in SEQ ID NO: 355, HCDR2 comprises the amino acid sequence set forth in SEQ ID NO: 350, HCDR3 comprises the amino acid sequence set forth in SEQ ID NO: 357, LCDR1 comprises the amino acid sequence set forth in SEQ ID NO: 375, LCDR2 comprises the amino acid sequence DDS, and LCDR3 comprises the amino acid sequence set forth in SEQ ID NO: 376;
(m) HCDR1 comprises the amino acid sequence set forth in SEQ ID NO: 349, HCDR2 comprises the amino acid sequence set forth in SEQ ID NO: 350, HCDR3 comprises the amino acid sequence set forth in SEQ ID NO: 351, LCDR1 comprises the amino acid sequence set forth in SEQ ID NO: 362, LCDR2 comprises the amino acid sequence DDV, and LCDR3 comprises the amino acid sequence set forth in SEQ ID NO: 377;
(n) HCDR1 comprises the amino acid sequence set forth in SEQ ID NO: 355, HCDR2 comprises the amino acid sequence set forth in SEQ ID NO: 356, HCDR3 comprises the amino acid sequence set forth in SEQ ID NO: 351, LCDR1 comprises the amino acid sequence set forth in SEQ ID NO: 369, LCDR2 comprises the amino acid sequence DDS, and LCDR3 comprises the amino acid sequence set forth in SEQ ID NO: 378;
(o) HCDR1 comprises the amino acid sequences amino acid sequence set forth in SEQ ID NO: 355, HCDR2 comprises the amino acid sequence set forth in SEQ ID NO: 356, HCDR3 comprises the amino acid sequence set forth in SEQ ID NO: 351, LCDR1 comprises the amino acid sequence set forth in SEQ ID NO: 369, LCDR2 comprises the amino acid sequence DDS, and LCDR3 comprises the amino acid sequence set forth in SEQ ID NO: 379;
(p) HCDR1 comprises the amino acid sequence set forth in SEQ ID NO: 349, HCDR2 comprises the amino acid sequence set forth in SEQ ID NO: 350, HCDR3 comprises the amino acid sequence set forth in SEQ ID NO: 351, LCDR1 comprises the amino acid sequence set forth in SEQ ID NO: 359, LCDR2 comprises the amino acid sequence DDV, and LCDR3 comprises the amino acid sequence set forth in SEQ ID NO: 380;
(q) HCDR1 comprises the amino acid sequence set forth in SEQ ID NO: 355, HCDR2 comprises the amino acid sequence set forth in SEQ ID NO: 356, HCDR3 comprises the amino acid sequence set forth in SEQ ID NO: 351, LCDR1 comprises the amino acid sequence set forth in SEQ ID NO: 369, LCDR2 comprises the amino acid sequence DDS, and LCDR3 comprises the amino acid sequence set forth in SEQ ID NO: 379;
(r) HCDR1 comprises the amino acid sequence set forth in SEQ ID NO: 355, HCDR2 comprises the amino acid sequence set forth in SEQ ID NO: 352, HCDR3 comprises the amino acid sequence set forth in SEQ ID NO: 351, LCDR1 comprises the amino acid sequence set forth in SEQ ID NO: 369, LCDR2 comprises the amino acid sequence DDS, and LCDR3 comprises the amino acid sequence set forth in SEQ ID NO: 381;
(s) HCDR1 comprises the amino acid sequence set forth in SEQ ID NO: 355, HCDR2 comprises the amino acid sequence set forth in SEQ ID NO: 350, HCDR3 comprises the amino acid sequence set forth in SEQ ID NO: 357, LCDR1 comprises the amino acid sequence set forth in SEQ ID NO: 369, LCDR2 comprises the amino acid sequence DDS, and LCDR3 comprises the amino acid sequence set forth in SEQ ID NO: 370;
(t) HCDR1 comprises the amino acid sequence set forth in SEQ ID NO: 355, HCDR2 comprises the amino acid sequence set forth in SEQ ID NO: 356, HCDR3 comprises the amino acid sequence set forth in SEQ ID NO: 351, LCDR1 comprises the amino acid sequence set forth in SEQ ID NO: 366, LCDR2 comprises the amino acid sequence DDS, and LCDR3 comprises the amino acid sequence set forth in SEQ ID NO: 382;
(u) HCDR1 comprises the amino acid sequence set forth in SEQ ID NO: 355, HCDR2 comprises the amino acid sequence set forth in SEQ ID NO: 350, HCDR3 comprises the amino acid sequence set forth in SEQ ID NO: 351, LCDR1 comprises the amino acid sequence set forth in SEQ ID NO: 369, LCDR2 comprises the amino acid sequence DDS, and LCDR3 comprises the amino acid sequence set forth in SEQ ID NO: 370;
(v) HCDR1 comprises the amino acid sequence set forth in SEQ ID NO: 355, HCDR2 comprises the amino acid sequence set forth in SEQ ID NO: 356, HCDR3 comprises the amino acid sequence set forth in SEQ ID NO: 351, LCDR1 comprises the amino acid sequence set forth in SEQ ID NO: 369, LCDR2 comprises the amino acid sequence DDS, and LCDR3 comprises the amino acid sequence set forth in SEQ ID NO: 383;
(w) HCDR1 comprises the amino acid sequence set forth in SEQ ID NO: 355, HCDR2 comprises the amino acid sequence set forth in SEQ ID NO: 352, HCDR3 comprises the amino acid sequence set forth in SEQ ID NO: 351, LCDR1 comprises the amino acid sequence set forth in SEQ ID NO: 366, LCDR2 comprises the amino acid sequence DDS, and LCDR3 comprises the amino acid sequence set forth in SEQ ID NO: 385;
(x) HCDR1 comprises the amino acid sequence set forth in SEQ ID NO: 355, HCDR2 comprises the amino acid sequence set forth in SEQ ID NO: 350, HCDR3 comprises the amino acid sequence set forth in SEQ ID NO: 351, LCDR1 comprises the amino acid sequence set forth in SEQ ID NO: 369, LCDR2 comprises the amino acid sequence DDS, and LCDR3 comprises the amino acid sequence set forth in SEQ ID NO: 386;
(y) HCDR1 comprises the amino acid sequence set forth in SEQ ID NO: 355, HCDR2 comprises the amino acid sequence set forth in SEQ ID NO: 352, HCDR3 comprises the amino acid sequence set forth in SEQ ID NO: 351, LCDR1 comprises the amino acid sequence set forth in SEQ ID NO: 369, LCDR2 comprises the amino acid sequence DDS, and LCDR3 comprises the amino acid sequence set forth in SEQ ID NO: 387;
(z) HCDR1 comprises the amino acid sequence set forth in SEQ ID NO: 355, HCDR2 comprises the amino acid sequence set forth in SEQ ID NO: 356, HCDR3 comprises the amino acid sequence set forth in SEQ ID NO: 351, LCDR1 comprises the amino acid sequence set forth in SEQ ID NO: 369, LCDR2 comprises the amino acid sequence DDS, and LCDR3 comprises the amino acid sequence set forth in SEQ ID NO: 388;
(aa) HCDR1 comprises the amino acid sequence set forth in SEQ ID NO: 355, HCDR2 comprises the amino acid sequence set forth in SEQ ID NO: 356, HCDR3 comprises the amino acid sequence set forth in SEQ ID NO: 351, LCDR1 comprises the amino acid sequence set forth in SEQ ID NO: 369, LCDR2 comprises the amino acid sequence DDS, and LCDR3 comprises the amino acid sequence set forth in SEQ ID NO: 389;
(bb) HCDR1 comprises the amino acid sequence set forth in SEQ ID NO: 355, HCDR2 comprises the amino acid sequence set forth in SEQ ID NO: 352, HCDR3 comprises the amino acid sequence set forth in SEQ ID NO: 351, LCDR1 comprises the amino acid sequence set forth in SEQ ID NO: 369, LCDR2 comprises the amino acid sequence DDS, and LCDR3 comprises the amino acid sequence set forth in SEQ ID NO: 379;
(cc) HCDR1 comprises the amino acid sequence set forth in SEQ ID NO: 355, HCDR2 comprises the amino acid sequence set forth in SEQ ID NO: 352, HCDR3 comprises the amino acid sequence set forth in SEQ ID NO: 351, LCDR1 comprises the amino acid sequence set forth in SEQ ID NO: 369, LCDR2 comprises the amino acid sequence DDS, and LCDR3 comprises the amino acid sequence set forth in SEQ ID NO: 390;
(dd) HCDR1 comprises the amino acid sequence set forth in SEQ ID NO: 355, HCDR2 comprises the amino acid sequence set forth in SEQ ID NO: 350, HCDR3 comprises the amino acid sequence set forth in SEQ ID NO: 351, LCDR1 comprises the amino acid sequence set forth in SEQ ID NO: 366, LCDR2 comprises the amino acid sequence DDS, and LCDR3 comprises the amino acid sequence set forth in SEQ ID NO: 391;
(ee) HCDR1 comprises the amino acid sequence set forth in SEQ ID NO: 355, HCDR2 comprises the amino acid sequence set forth in SEQ ID NO: 350, HCDR3 comprises the amino acid sequence set forth in SEQ ID NO: 351, LCDR1 comprises the amino acid sequence set forth in SEQ ID NO: 375, LCDR2 comprises the amino acid sequence DDS, and LCDR3 comprises the amino acid sequence set forth in SEQ ID NO: 392;
(ff) HCDR1 comprises the amino acid sequence set forth in SEQ ID NO: 355, HCDR2 comprises the amino acid sequence set forth in SEQ ID NO: 354, HCDR3 comprises the amino acid sequence set forth in SEQ ID NO: 351, LCDR1 comprises the amino acid sequence set forth in SEQ ID NO: 369, LCDR2 comprises the amino acid sequence DDS, and LCDR3 comprises the amino acid sequence set forth in SEQ ID NO: 393;
(gg) HCDR1 comprises the amino acid sequence set forth in SEQ ID NO: 355, HCDR2 comprises the amino acid sequence set forth in SEQ ID NO: 356, HCDR3 comprises the amino acid sequence set forth in SEQ ID NO: 351, LCDR1 comprises the amino acid sequence set forth in SEQ ID NO: 369, LCDR2 comprises the amino acid sequence DDS, and LCDR3 comprises the amino acid sequence set forth in SEQ ID NO: 394;
(hh) HCDR1 comprises the amino acid sequence set forth in SEQ ID NO: 355, HCDR2 comprises the amino acid sequence set forth in SEQ ID NO: 356, HCDR3 comprises the amino acid sequence set forth in SEQ ID NO: 351, LCDR1 comprises the amino acid sequence set forth in SEQ ID NO: 369, LCDR2 comprises the amino acid sequence DDS, and LCDR3 comprises the amino acid sequence set forth in SEQ ID NO: 395;
(ii) HCDR1 comprises the amino acid sequence set forth in SEQ ID NO: 355, HCDR2 comprises the amino acid sequence set forth in SEQ ID NO: 350, HCDR3 comprises the amino acid sequence set forth in SEQ ID NO: 351, LCDR1 comprises the amino acid sequence set forth in SEQ ID NO: 369, LCDR2 comprises the amino acid sequence DDS, and LCDR3 comprises the amino acid sequence set forth in SEQ ID NO: 396;
(jj) HCDR1 comprises the amino acid sequence set forth in SEQ ID NO: 355, HCDR2 comprises the amino acid sequence set forth in SEQ ID NO: 352, HCDR3 comprises the amino acid sequence set forth in SEQ ID NO: 351, LCDR1 comprises the amino acid sequence set forth in SEQ ID NO: 369, LCDR2 comprises the amino acid sequence DDS, and LCDR3 comprises the amino acid sequence set forth in SEQ ID NO: 397;
(kk) HCDR1 comprises the amino acid sequence set forth in SEQ ID NO: 355, HCDR2 comprises the amino acid sequence set forth in SEQ ID NO: 350, HCDR3 comprises the amino acid sequence set forth in SEQ ID NO: 351, LCDR1 comprises the amino acid sequence set forth in SEQ ID NO: 369, LCDR2 comprises the amino acid sequence DDS, and LCDR3 comprises the amino acid sequence set forth in SEQ ID NO: 398;
(ll) HCDR1 comprises the amino acid sequence set forth in SEQ ID NO: 355, HCDR2 comprises the amino acid sequence set forth in SEQ ID NO: 350, HCDR3 comprises the amino acid sequence set forth in SEQ ID NO: 351, LCDR1 comprises the amino acid sequence set forth in SEQ ID NO: 369, LCDR2 comprises the amino acid sequence DDS, and LCDR3 comprises the amino acid sequence set forth in SEQ ID NO: 388;
(mm) HCDR1 comprises the amino acid sequence set forth in SEQ ID NO: 355, HCDR2 comprises the amino acid sequence set forth in SEQ ID NO: 350, HCDR3 comprises the amino acid sequence set forth in SEQ ID NO: 351, LCDR1 comprises the amino acid sequence set forth in SEQ ID NO: 369, LCDR2 comprises the amino acid sequence DDS, and LCDR3 comprises the amino acid sequence set forth in SEQ ID NO: 394;
(nn) HCDR1 comprises the amino acid sequence set forth in SEQ ID NO: 355, HCDR2 comprises the amino acid sequence set forth in SEQ ID NO: 350, HCDR3 comprises the amino acid sequence set forth in SEQ ID NO: 351, LCDR1 comprises the amino acid sequence set forth in SEQ ID NO: 375, LCDR2 comprises the amino acid sequence DDS, and LCDR3 comprises the amino acid sequence set forth in SEQ ID NO: 398;
(oo) HCDR1 comprises the amino acid sequence set forth in SEQ ID NO: 355, HCDR2 comprises the amino acid sequence set forth in SEQ ID NO: 352, HCDR3 comprises the amino acid sequence set forth in SEQ ID NO: 357, LCDR1 comprises the amino acid sequence set forth in SEQ ID NO: 375, LCDR2 comprises the amino acid sequence DDS, and LCDR3 comprises the amino acid sequence set forth in SEQ ID NO: 399;
(pp) HCDR1 is set forth in SEQ ID NO: 355, HCDR2 comprises the amino acid sequence set forth in SEQ ID NO: 350, HCDR3 comprises the amino acid sequence set forth in SEQ ID NO: 351, LCDR1 comprises the amino acid sequence set forth in SEQ ID NO: 375, LCDR2 comprises the amino acid sequence DDS, and LCDR3 comprises the amino acid sequence set forth in SEQ ID NO: 400;
(qq) HCDR1 comprises the amino acid sequence set forth in SEQ ID NO: 355, HCDR2 comprises the amino acid sequence set forth in SEQ ID NO: 350, HCDR3 comprises the amino acid sequence set forth in SEQ ID NO: 357, LCDR1 comprises the amino acid sequence set forth in SEQ ID NO: 375, LCDR2 comprises the amino acid sequence DDS, and LCDR3 comprises the amino acid sequence set forth in SEQ ID NO: 398;
(rr) HCDR1 comprises the amino acid sequence set forth in SEQ ID NO: 349, HCDR2 comprises the amino acid sequence set forth in SEQ ID NO: 350, HCDR3 comprises the amino acid sequence set forth in SEQ ID NO: 351, LCDR1 comprises the amino acid sequence set forth in SEQ ID NO: 369, LCDR2 comprises the amino acid sequence DDS, and LCDR3 comprises the amino acid sequence set forth in SEQ ID NO: 378;
(ss) HCDR1 comprises the amino acid sequence set forth in SEQ ID NO: 355, HCDR2 comprises the amino acid sequence set forth in SEQ ID NO: 350, HCDR3 comprises the amino acid sequence set forth in SEQ ID NO: 351, LCDR1 comprises the amino acid sequence set forth in SEQ ID NO: 375, LCDR2 comprises the amino acid sequence DDS, and LCDR3 comprises the amino acid sequence set forth in SEQ ID NO: 401;
(tt) HCDR1 comprises the amino acid sequence set forth in SEQ ID NO: 355, HCDR2 comprises the amino acid sequence set forth in SEQ ID NO: 356, HCDR3 comprises the amino acid sequence set forth in SEQ ID NO: 358, LCDR1 comprises the amino acid sequence set forth in SEQ ID NO: 369, LCDR2 comprises the amino acid sequence DDS, and LCDR3 comprises the amino acid sequence set forth in SEQ ID NO: 402;
(uu) HCDR1 comprises the amino acid sequence set forth in SEQ ID NO: 349, HCDR2 comprises the amino acid sequence set forth in SEQ ID NO: 356, HCDR3 comprises the amino acid sequence set forth in SEQ ID NO: 358, LCDR1 comprises the amino acid sequence set forth in SEQ ID NO: 369, LCDR2 comprises the amino acid sequence DDS, and LCDR3 comprises the amino acid sequence set forth in SEQ ID NO: 403;
(vv) HCDR1 comprises the amino acid sequence set forth in SEQ ID NO: 355, HCDR2 comprises the amino acid sequence set forth in SEQ ID NO: 356, HCDR3 comprises the amino acid sequence set forth in SEQ ID NO: 358, LCDR1 comprises the amino acid sequence set forth in SEQ ID NO: 375, LCDR2 comprises the amino acid sequence DDS, and LCDR3 comprises the amino acid sequence set forth in SEQ ID NO: 376;
(ww) HCDR1 comprises the amino acid sequence set forth in SEQ ID NO: 355, HCDR2 comprises the amino acid sequence set forth in SEQ ID NO: 350, HCDR3 comprises the amino acid sequence set forth in SEQ ID NO: 351, LCDR1 comprises the amino acid sequence set forth in SEQ ID NO: 369, LCDR2 comprises the amino acid sequence DDS, and LCDR3 comprises the amino acid sequence set forth in SEQ ID NO: 402;
(xx) HCDR1 comprises the amino acid sequence set forth in SEQ ID NO: 349, HCDR2 comprises the amino acid sequence set forth in SEQ ID NO: 350, HCDR3 comprises the amino acid sequence set forth in SEQ ID NO: 353, LCDR1 comprises the amino acid sequence set forth in SEQ ID NO: 359, LCDR2 comprises the amino acid sequence DDV, and LCDR3 comprises the amino acid sequence set forth in SEQ ID NO: 404;
(yy) HCDR1 comprises the amino acid sequence set forth in SEQ ID NO: 355, HCDR2 comprises the amino acid sequence set forth in SEQ ID NO: 350, HCDR3 comprises the amino acid sequence set forth in SEQ ID NO: 357, LCDR1 comprises the amino acid sequence set forth in SEQ ID NO: 375, LCDR2 comprises the amino acid sequence DDS, and LCDR3 comprises the amino acid sequence set forth in SEQ ID NO: 405;
(zz) HCDR1 comprises the amino acid sequence set forth in SEQ ID NO: 355, HCDR2 comprises the amino acid sequence set forth in SEQ ID NO: 350, HCDR3 comprises the amino acid sequence set forth in SEQ ID NO: 351, LCDR1 comprises the amino acid sequence set forth in SEQ ID NO: 366, LCDR2 comprises the amino acid sequence DDS, and LCDR3 comprises the amino acid sequence set forth in SEQ ID NO: 406;
(aaa) HCDR1 comprises the amino acid sequence set forth in SEQ ID NO: 355, HCDR2 comprises the amino acid sequence set forth in SEQ ID NO: 350, HCDR3 comprises the amino acid sequence set forth in SEQ ID NO: 351, LCDR1 comprises the amino acid sequence set forth in SEQ ID NO: 375, LCDR2 comprises the amino acid sequence DDS, and LCDR3 comprises the amino acid sequence set forth in SEQ ID NO: 407; and
(bbb) HCDR1 comprises the amino acid sequence set forth in SEQ ID NO: 355, HCDR2 comprises the amino acid sequence set forth in SEQ ID NO: 354, HCDR3 comprises the amino acid sequence set forth in SEQ ID NO: 351, LCDR1 comprises the amino acid sequence set forth in SEQ ID NO: 369, LCDR2 comprises the amino acid sequence DDS, and LCDR3 comprises the amino acid sequence set forth in SEQ ID NO: 379.

19. The method of claim 17, wherein said disease or condition is an allergic or respiratory condition, an inflammatory or autoimmune condition, or tumors or cancers.

20. The method of claim 17, wherein said disease or condition is asthma, allergic asthma, nonallergic asthma, severe asthma, mild asthma, chronic obstructive pulmonary disease (COPD), a condition involving airway inflammation, cystic fibrosis, allergic lung disease, airway hyperresponsiveness, goblet cell metaplasia, mucus hypersecretion, airway remodeling, pulmonary fibrosis, atopic dermatitis, urticaria, eczema, allergic enterogastritis, allergic rhinitis, inflammatory bowel diseases, liver cirrhosis or fibrosis, or a combination thereof.

21. A method of treating a subject suffering from a disease or condition, said method comprises administering to said subject a composition comprising an isolated anti-IL-13 anti-TSLP dual binding antibody comprising a heavy chain (HC) comprising a heavy chain variable region (VH) and a light chain (LC) comprising a light chain variable region (VL), wherein said HC comprises a L234A L235A (LALA) mutation or a L234A L235AP329G (LALAPG) mutation, and wherein amino acid sequences of said VH and VL are set forth in SEQ ID NOs: 337 and 338, respectively.

22. The method of claim 21, wherein amino acid sequences of said HC and LC are set forth in SEQ ID NOs: 410 and 409, respectively, or SEQ ID NOs: 408 and 409, respectively.
